(19) United States
(12) Patent Application Publication
(10) Pub. No.: US 2013/0078244 A1
(43) Pub. Date: Mar. 28, 2013

(54) METHODS FOR DETECTING AND REGULATING ALOPECIA AREATA AND GENE COHORTS THEREOF

(71) Applicants: ANGELA M. CHRISTIANO, MAHWAH, NJ (US); Raphael Clynes, West Nyack, NY (US)

(72) Inventors: ANGELA M. CHRISTIANO, MAHWAH, NJ (US); Raphael Clynes, West Nyack, NY (US)

(21) Appl. No.: 13/540,088

(22) Filed: Jul. 2, 2012

Related U.S. Application Data
(63) Continuation-in-part of application No. PCT/US2010/062641, filed on Dec. 31, 2010.
(60) Provisional application No. 61/291,645, filed on Dec. 31, 2009.

(51) Int. Cl. C07K 16/18; C12N 15/113

(52) U.S. Cl. CPC C07K 16/18; C12N 15/113
USPC 424/134.1; 506/2; 506/16; 536/24.33; 530/389.8; 536/24.5; 435/7.21; 514/44 R; 435/7.92; 435/6.12; 514/44 A; 424/172.1

(57) ABSTRACT

The invention provides for methods for controlling hair growth by administering a HLDGC modulating compound to a subject. The invention further provides for a method for screening compounds that bind to and modulate polypeptides encoded by HLDGC genes. The invention also provides methods of detecting the presence of or a predisposition to a hair-loss disorder in a human subject as well as methods of treating such disorders.

This application is a continuation-in-part of International Application No. PCT/US2010/062641, filed Dec. 31, 2010, which claims priority to U.S. Provisional Application Ser. No. 61/291,645, filed Dec. 31, 2009, the contents of each of which are hereby incorporated by reference in their entireties.

### GOVERNMENT INTERESTS

This invention was made with government support under RO1 AR56016 awarded by the National Institutes of Health/National Institute of Arthritis and Musculoskeletal and Skin Diseases. The United States Government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Alopecia Areata (AA) is one of the most highly prevalent autoimmune diseases, leading to hair loss due to the collapse of immune privilege of the hair follicle and subsequent autoimmune destruction. AA is a skin disease which leads to hair loss on the scalp and elsewhere. In some severe cases, it can progress to complete loss of hair on the head or body. Although Alopecia Areata is believed to be caused by autoimmunity, the gene level diagnosis and treatment are seldom reported. The genetic basis of AA is largely unknown.

### SUMMARY OF THE INVENTION

The invention provides methods for controlling hair growth (such as inducing hair growth, or inhibiting hair growth) by administering a HLDGC modulating compound to a subject. The invention further provides for methods for screening compounds that bind to and modulate polypeptides encoded by HLDGC genes. The invention also provides methods of detecting the presence of or a predisposition to a hair-loss disorder in a human subject as well as methods of treating such disorders.

In one aspect, the invention encompasses a method for detecting the presence of or a predisposition to a hair-loss disorder in a human subject where the method comprises obtaining a biological sample from a human subject; and detecting whether or not there is an alteration in the level of expression of an mRNA or a protein encoded by a HLDGC gene in the subject as compared to the level of expression in a subject not afflicted with a hair-loss disorder. In on embodiment, the detecting comprises determining whether mRNA expression or protein expression of the HLDGC gene is increased or decreased as compared to expression in a normal sample. In another embodiment, the detecting comprises determining in the sample whether expression of at least 2 HLDGC proteins, at least 3 HLDGC proteins, at least 4 HLDGC proteins, at least 5 HLDGC proteins, at least 6 HLDGC proteins, at least 6 HLDGC proteins, at least 7 HLDGC proteins, or at least 8 HLDGC proteins is increased or decreased as compared to expression in a normal sample. In some embodiments, the detecting comprises determining in the sample whether expression of at least 2 HLDGC mRNAs, at least 3 HLDGC mRNAs, at least 4 HLDGC mRNAs, at least 5 HLDGC mRNAs, at least 6 HLDGC mRNAs, at least 6 HLDGC mRNAs, at least 7 HLDGC mRNAs, or at least 8 HLDGC mRNAs is increased or decreased as compared to expression in a normal sample. In one embodiment, an increase in the expression of at least 2 HLDGC genes, at least 3 HLDGC genes, at least 4 HLDGC genes, at least 5 HLDGC genes, at least 6 HLDGC genes, at least 7 HLDGC genes, or at least 8 HLDGC genes indicates a predisposition to or presence of a hair-loss disorder in the subject. In another embodiment, a decrease in the expression of at least 2 HLDGC genes, at least 3 HLDGC genes, at least 4 HLDGC genes, at least 5 HLDGC genes, at least 6 HLDGC genes, at least 7 HLDGC genes, or at least 8 HLDGC genes indicates a predisposition to or presence of a hair-loss disorder in the subject. In one embodiment, the mRNA expression or protein expression level in the subject is about 5-fold increased, about 10-fold increased, about 15-fold increased, about 20-fold increased, about 25-fold increased, about 30-fold increased, about 35-fold increased, about 40-fold increased, about 45-fold increased, about 50-fold increased, about 55-fold increased, about 60-fold increased, about 65-fold increased, about 70-fold increased, about 75-fold increased, about 80-fold increased, about 85-fold increased, about 90-fold increased, about 95-fold increased, or is 100-fold increased, as compared to that in the normal sample. In some embodiments, the he mRNA expression or protein expression level in the subject is at least about 100-fold increased, at least about 200-fold increased, at least about 300-fold increased, at least about 400-fold increased, or is at least about 500-fold increased, as compared to that in the normal sample. In further embodiments, the mRNA expression or protein expression level of the HLDGC gene in the subject is about 5-fold to about 70-fold increased, as compared to that in the normal sample. In other embodiments, the mRNA or protein expression level of the HLDGC gene in the subject is about 5-fold to about 90-fold increased, as compared to that in the normal sample. In one embodiment, the mRNA expression or protein expression level in the subject is about 5-fold decreased, about 10-fold decreased, about 15-fold decreased, about 20-fold decreased, about 25-fold decreased, about 30-fold decreased, about 35-fold decreased, about 40-fold decreased, about 45-fold decreased, about 50-fold decreased, about 55-fold decreased, about 60-fold decreased, about 65-fold decreased, about 70-fold decreased, about 75-fold decreased, about 80-fold decreased, about 85-fold decreased, about 90-fold decreased, about 95-fold decreased, or is 100-fold decreased, as compared to that in the normal sample. In some embodiments, the mRNA expression or protein expression level in the subject is at least about 100-fold decreased, as compared to that in the normal sample. In some embodiments, the mRNA or protein expression level of the HLDGC gene in the subject is about 5-fold to about 70-fold decreased, as compared to that in the normal sample. In yet other embodiments, the mRNA or protein expression level of the HLDGC gene in the subject is about 5-fold to about 90-fold decreased, as compared to that in the normal sample. In further embodiments, the detecting comprises gene sequencing, selective hybridization, selective amplification, gene expression analysis, or a combination thereof. In another embodiment, the hair-loss disorder comprises androgenetic alopecia, alopecia areata, telogen effluvium, alopecia totalis, hypotrichosis, hereditary hypotrichosis simplex, or alopecia universalis. In one embodiment, the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In some embodiments, the HLA Region residing gene is selected from the group consisting of a gene of the HLA Class I Region, a gene of the HLA Class II Region, PTPN22, and AIRE. In another embodiment, the HLA Class I Region gene is HLA-A, HLA-B, HLA-C, HLA-DQB1, HLA-DRB1, MICA, MICB, HLA-G, or NOTCH4. In a further embodiment, the HLA Class II Region gene is HLA-DOB, HLA-DQA1, HLA-DQA2, HLA-DQB2, TAP2, or HLA-DRA.

In one aspect, the invention encompasses a method for detecting the presence of or a predisposition to a hair-loss disorder in a human subject where the method comprises obtaining a biological sample from a human subject; and detecting the presence of one or more single nucleotide polymorphisms (SNPs) in a chromosome region containing a HLDGC gene in the subject, wherein the SNP is selected from the SNPs listed in Table 2. In one embodiment, the chromosome region comprises region 2q33.2, region 4q27, region 4q31.3, region 5p13.1, region 6q25.1, region 9q31.1, region 10p15.1, region 11q13, region 12813, region 6p21.32, or a combination thereof. In other embodiments, the single nucleotide polymorphism is selected from the group consisting of rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, and rs6910071. In another embodiment, the detecting comprises gene sequencing, selective hybridization, selective amplification, gene expression analysis, or a combination thereof. In a further embodiment, the hair-loss disorder comprises androgenetic alopecia, alopecia areata, telogen effluvium, alopecia totalis, hypotrichosis, hereditary hypotrichosis simplex, or alopecia universalis.

One aspect of the invention encompasses a cDNA- or oligonucleotide-microarray for diagnosis of a hair-loss disorder, wherein the microarray comprises SEQ ID NOS: 2, 4, 6, 8, 10, 12, 14, 16, 18, 20, 22, 24, or a combination thereof.

Another aspect of the invention provides for a cDNA- or oligonucleotide-microarray for diagnosis of a hair-loss disorder, wherein the microarray comprises SNPs listed in Table 2.

An aspect of the invention encompasses a cDNA- or oligonucleotide-microarray for diagnosis of a hair-loss disorder, wherein the microarray comprises SNPs rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, rs6910071, or a combination of SNPs listed herein.

An aspect of the invention encompasses methods for determining whether a subject exhibits a predisposition to a hair-loss disorder using any one of the microarrays described herein. The methods comprise obtaining a nucleic acid sample from the subject; performing a hybridization to form a double-stranded nucleic acid between the nucleic acid sample and a probe; and detecting the hybridization. In one embodiment, the hybridization is detected radioactively, by fluorescence, or electrically. In another embodiment, the nucleic acid sample comprises DNA or RNA. In a further embodiment, the nucleic acid sample is amplified.

One aspect of the invention encompasses a diagnostic kit for determining whether a sample from a subject exhibits a predisposition to a hair-loss disorder, the kit comprising a cDNA- or oligonucleotide-microarray described herein.

An aspect of the invention provides for a diagnostic kit for determining whether a sample from a subject exhibits increased or decreased expression of at least 2 or more HLDGC genes, the kit comprising a nucleic acid primer that specifically hybridizes to one or more HLDGC genes. In one embodiment, the primer comprises a nucleotide sequence selected from the group consisting of SEQ ID NOS: 25-40 in Table 9. In a further embodiment, the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In some embodiments, the HLA Region residing gene is selected from the group consisting of a gene of the HLA Class I Region, a gene of the HLA Class II Region, PTPN22, and AIRE. In other embodiments, the HLA Class I Region gene is HLA-A, HLA-B, HLA-C, HLA-DQB1, HLA-DRB1, MICA, MICB, HLA-G, or NOTCH4. In further embodiments, the HLA Class II Region gene is HLA-DOB, HLA-DQA1, HLA-DQA2, HLA-DQB2, TAP2, or HLA-DRA.

An aspect of the invention encompasses a diagnostic kit for determining whether a sample from a subject exhibits a predisposition to a hair-loss disorder, the kit comprising a nucleic acid primer that specifically hybridizes to a single nucleotide polymorphism (SNP) in a chromosome region containing a HLDGC gene, wherein the primer will prime a polymerase reaction only when a SNP of Table 2 is present. In one embodiment, the primer comprises a nucleotide sequence selected from the group consisting of SEQ ID NOS: 25-40 in Table 9. In another embodiment, the SNP is selected from the group consisting of rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, and rs6910071. In a further embodiment, the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In some embodiments, the HLA Region residing gene is selected from the group consisting of a gene of the HLA Class I Region, a gene of the HLA Class II Region, PTPN22, and AIRE. In other embodiments, the HLA Class I Region gene is HLA-A, HLA-B, HLA-C, HLA-DQB1, HLA-DRB1, MICA, MICB, HLA-G, or NOTCH4. In further embodiments, the HLA Class II Region gene is HLA-DOB, HLA-DQA1, HLA-DQA2, HLA-DQB2, TAP2, or HLA-DRA.

An aspect of the encompasses a composition for modulating HLDGC protein expression or activity in a subject wherein the composition comprises an antibody that specifically binds to the HLDGC protein or a fragment thereof; an antisense RNA that specifically inhibits expression of a HLDGC gene that encodes the HLDGC protein; or a siRNA that specifically targets the HLDGC gene encoding the HLDGC protein. In one embodiment, the siRNA comprises a nucleic acid sequence comprising any one sequence of SEQ ID NOS: 41-6152. In another embodiment, the siRNA is directed to ULBP3, ULBP6, or PRDX5. In some embodiments, the antibody is directed to ULBP3, ULBP6, or PRDX5.

An aspect of the invention provides for a method for inducing hair growth in a subject where the method comprises administering to the subject an effective amount of a HLDGC modulating compound, thereby controlling hair growth in the subject. The effective amount of the composition would result in hair growth in the subject. In one embodiment, the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In another embodiment, the HLA Region residing gene is selected from the group consisting of a gene of the HLA Class I Region, a gene of the HLA Class II Region, PTPN22, and AIRE. In some embodiments, the HLA Class I Region gene is HLA-A, HLA-B, HLA-C, HLA-DQB1, HLA-DRB1, MICA, MICB, HLA-G, and NOTCH4. In other embodiments, the HLA Class II Region gene is HLA-DOB, HLA-DQA1, HLA-DQA2, HLA-DQB2, TAP2, and HLA-DRA. In further embodiments, the modulating compound comprises an antibody that specifically binds to a the HLDGC protein or a fragment thereof; an antisense RNA that specifically inhibits expression of a HLDGC gene that encodes the HLDGC protein; or a siRNA that specifically targets the HLDGC gene encoding the HLDGC protein. In other embodiments, the modulating compound is a functional HLDGC gene that encodes the HLDGC protein, or a functional HLDGC protein. In some embodiments, the subject is afflicted with a hair-loss disorder. In other embodiments, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In some embodiments, the modulating compound may also inhibit hair growth, thus it can be used for treatment of hair growth disorders, such as hypertrichosis.

The invention provides for a method for identifying a compound useful for treating alopecia areata or an immune disorder where the method comprises contacting a NKG2D-positive (+) cell with a test agent in vitro in the presence of a NKG2D ligand; and determining whether the test agent altered the cell response to the ligand binding to the NKG2D receptor as compared to an NKG2D+ cell contacted with the NKG2D ligand in the absence of the test agent, thereby identifying a compound useful for treating alopecia areata or an immune disorder. In one embodiment, the test agent specifically binds a NKG2D ligand. In another embodiment, the NKG2D ligand comprises ULBP1, ULBP2, ULBP3, ULBP4, ULBP5, ULBP6, or a combination thereof. In some embodiments, the determining comprises measuring ligand-induced NKG2D activation of the NKG2D+ cell. In further embodiments, the compound decreases downstream receptor signaling of the NKG2D protein. In other embodiments, measuring ligand-induced NKG2D activation comprises one or more of measuring NKG2D internalization, DAP10 phosphorylation, p85 PI3 kinase activity, Akt kinase activity, production of IFNγ, and cytolysis of a NKG2D-ligand+ target cell. In some embodiments, the NKG2D+ cell is a lymphocyte or a hair follicle cell. In another embodiment, the lymphocyte is a Natural Killer cell, γδ-TcR+ T cell, CD8+ T cell, a CD4+ T cell, or a B cell.

One aspect of the invention encompasses a method of treating a hair-loss disorder in a mammalian subject in need thereof, the method comprising administering to the subject an antibody or antibody fragment that binds ULBP3, ULBP6, or PRDX5. The therapeutic amount of the composition would result in hair growth in the subject. In another embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

One aspect of the invention provides for methods of treating a hair-loss disorder in a mammalian subject in need thereof, the method comprising administering to the subject an RNA molecule that specifically targets the PRDX5 gene encoding the PRDX5 protein. The therapeutic amount of the composition would result in hair growth in the subject. In one embodiment, the RNA molecule is an antisense RNA or a siRNA. In another embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

One aspect of the invention provides for methods of treating a hair-loss disorder in a mammalian subject in need thereof, the method comprising administering to the subject an RNA molecule that specifically targets the ULBP3 gene encoding the ULBP3 protein. The therapeutic amount of the composition would result in hair growth in the subject. In one embodiment, the RNA molecule is an antisense RNA or a siRNA. In another embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

One aspect of the invention provides for methods of treating a hair-loss disorder in a mammalian subject in need thereof, the method comprising administering to the subject an RNA molecule that specifically targets the ULBP6 gene encoding the ULBP6 protein. The therapeutic amount of the composition would result in hair growth in the subject. In one embodiment, the RNA molecule is an antisense RNA or a siRNA. In another embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

An aspect of the invention encompasses a method for treating or preventing a hair-loss disorder in a mammalian subject in need thereof, the method comprising administering to the subject a therapeutic amount of a pharmaceutical composition comprising a functional HLDGC gene that encodes the HLDGC protein, or a functional HLDGC protein, thereby treating or preventing a hair-loss disorder. The therapeutic amount of the composition would result in hair growth in the subject. In a further embodiment, the administering comprises a subcutaneous, intra-muscular, intra-peritoneal, or intravenous injection; an infusion; oral, nasal, or topical delivery; or a combination thereof. In one embodiment, the administering comprises delivery of a functional HLDGC gene that encodes the HLDGC protein, or a functional HLDGC protein to the epidermis or dermis of the subject. In some embodiments, the administering occurs daily, weekly, twice weekly, monthly, twice monthly, or yearly. In one embodiment, the HLDGC gene or protein is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In one embodiment, the HLDGC gene or protein is PRDX5. In another embodiment, the HLA Region residing gene is selected from the group consisting of a gene of the HLA Class I Region, a gene of the HLA Class II Region, PTPN22, and AIRE. In a further embodiment, the HLA Class I Region gene is HLA-A, HLA-B, HLA-C, HLA-DQB1, HLA-DRB1, MICA, MICB, HLA-G, and NOTCH4. In some embodiments, the HLA Class II Region gene is HLA-DOB, HLA-DQA1, HLA-DQA2, HLA-DQB2, TAP2, and HLA-DRA. In other embodiments, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis.

An aspect of the invention provides for treating or preventing a hair-loss disorder in a mammalian subject in need thereof, the method comprising administering to the subject a therapeutic amount of a pharmaceutical composition comprising the composition of an antibody that specifically binds to the HLDGC protein or a fragment thereof; an antisense RNA that specifically inhibits expression of a HLDGC gene that encodes the HLDGC protein; or a siRNA that specifically targets the HLDGC gene encoding the HLDGC protein, thereby treating or preventing a hair-loss disorder. The therapeutic amount of the composition would result in hair growth in the subject. In one embodiment, the siRNA comprises a nucleic acid sequence comprising any one sequence of SEQ ID NOS: 41-6152. In a further embodiment, the administering comprises a subcutaneous, intra-muscular, intra-peritoneal, or intravenous injection; an infusion; oral, nasal, or topical delivery; or a combination thereof. In another embodiment, the administering comprises delivery of the composition to the epidermis or dermis of the subject. In some embodiments, the administering occurs daily, weekly, twice weekly, monthly, twice monthly, or yearly. In one embodiment, the HLDGC gene or protein is CTLA-4, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In one embodiment, the HLDGC gene or protein is ULBP3. In one embodiment, the HLDGC gene is ULBP6. In another embodiment, the HLA Region residing gene is selected from the group consisting of a gene of the HLA Class I Region, a gene of the HLA Class II Region, PTPN22, and AIRE. In a further embodiment, the HLA Class I Region gene is HLA-A, HLA-B, HLA-C, HLA-DQB1, HLA-DRB1, MICA, MICB, HLA-G, and NOTCH4. In some embodiments, the HLA Class II Region gene is HLA-DOB, HLA-DQA1, HLA-DQA2, HLA-DQB2, TAP2, and HLA-DRA. In other embodiments, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis.

One aspect of the invention provides for methods of treating a hair-loss disorder in a mammalian subject in need thereof, the method comprising administering to the subject a therapeutic amount of a pharmaceutical composition comprising a functional PRDX5 gene that encodes the PRDX5 protein, or a functional PRDX5 protein. The therapeutic amount of the composition would result in hair growth in the subject. In another embodiment, the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis. In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

### BRIEF DESCRIPTION OF THE FIGURES

The patent or application file contains at least one drawing executed in color. Copies of this patent or patent application publication with color drawings will be provided by the Office upon request and payment of the necessary fee.

The original color versions of FIGS. 1-7 can be viewed in Petukhova et al., Nature. 2010 Jul. 1; 466(7302):113-7 (including the accompanying Supplementary Information available in the on-line version of the manuscript available on the Nature web site). For the purposes of the this application, the contents of Petukhova et al., Nature. 2010 Jul. 1; 466(7302):113-7, including the accompanying “Supplementary Information,” are herein incorporated by reference.

FIG. 1 are photographic images of clinical manifestations of AA. In the upper panels (FIGS. 1A-B), patients with AA multiplex. In FIG. 1B, the patient is in regrowth phase. For patients with alopecia universalis (AU), there is a complete lack of body hair and scalp hair (FIG. 1C), while patients with alopecia totalis only lack scalp hair (FIG. 1D). In FIG. 1D, hair regrowth is observed in the parietal region, while no regrowth in either occipital or temporal regions is evident.

FIG. 2 is a graph of a Manhattan plot of the joint analysis of the discovery genomewide association study (GWAS) and the replication GWAS. Results are plotted as the -log transformed p-values from a genotypic association test controlled for residual population stratification as a function of the position in the genome. Odd chromosomes are in gray and even chromosomes are in black. Ten genomic regions contain SNPs that exceed the genome-wide significance threshold of 5×10⁻⁷ (black line).

FIGS. 3A-P are graphs of the linkage disequilibrium (LD) structure and haplotype organization of the implicated regions from GWAS. In all graphs, the genome-wide significance threshold (5×10⁻⁷) is indicated by a black dotted line. Results from the eight regions are aligned with LD maps (FIGS. 3A, 3C, 3E, 3G, 3I, 3K, 3M, 3O) and transcript maps (FIGS. 3B, 3D, 3F, 3H, 3J, 3L, 3N, 3P): chromosome 2q33 (FIGS. 3A, 3B), 4q26-27 (FIGS. 3C, 3D), 6p21.3 (FIGS. 3E, 3F), 6q25 (FIGS. 3G, 3H), 9q31.1 (FIGS. 3I, 3J), 10p15-p16 (FIGS. 3K, 3L), 11q13 (FIGS. 3M, 3N), and 12q13 (FIGS. 3O, 3P). For the plots with the LD maps, dark grey indicates high LD as measured by D′. For the plots with the transcript maps, SNPs that do not reach significance are in grey while significantly associated SNPs are in color, coded by the risk haplotypes. For example in FIG. 3B, conditioning on any of the black SNPs, will reduce evidence for association of the other black SNPs, but will not affect any of the white SNPs. On chromosome 6p in the HLA, significantly associated SNPs can be organized into at least five distinct haplotypes. Pair-wise LD was measured by r² for the most significant SNP in each haplotype and defines the LD block that is demonstrating association.

FIGS. 3Q-R are graphs of the cumulative effect of risk haplotypes is indicated by the distribution of the genetic liability index (GLI) in cases and controls. Given that we were able to reduce the redundancy of 141 significantly associated SNPs within the ten regions to 18 independent effects, we sought to determine if the effects of the risk alleles are cumulative. We chose one SNP from each haplotype to serve as a proxy for the haplotype, choosing the most significantly associated SNP. The GLI is calculated as the sum of the risk alleles carried by an individual. The GLI distribution changes as a function of phenotype. No control sample carried more than 16 risk alleles in total while no case sample carried less than 4 risk alleles. As the number of risk alleles in an individual increases, the proportion affected by AA increases. The distribution of GLI in cases (dark grey) and controls (light grey) is shown in FIG. 3Q. The conditional probability of phenotype given a number of risk alleles is shown in FIG. 3R (AA in gray, control in black).

FIGS. 4A-L are photomicrographs showing ULBP3 expression and immune cell infiltration of AA hair follicles. FIGS. 4A-B show low levels of expression of ULBP3 in the dermal papilla of hair follicles from two unrelated, unaffected individuals. FIGS. 4C-D show massive upregulation of ULBP3 expression in the dermal sheath of hair follicles from two unrelated patients with AA in the early stages of disease. FIGS. 4E-F show the absence of immune infiltration in two control hair follicles. FIG. 4G shows hematoxylin and eosin staining of AA hair follicle. DS, dermal sheath; Mx, matrix; DP, dermal papilla. FIGS. 4H-I show immunofluorescence analysis using CD3 and CD8 cell surface markers for T cell lineages. Note the marked inflammatory infiltrate in the dermal sheath of two affected AA hair follicles. FIGS. 4J-L show double-immunofluorescence analysis with anti-CD3 and anti-CD8 antibodies. The merged image of FIG. 4J and FIG. 4K shows infiltration of CD3+CD8+ T cells in the dermal sheath of AA hair follicle (FIG. 4L). FIG. 4D and FIGS. 4G-L are serial sections of the same hair follicle of an affected individual. The cells were counterstained with DAPI (FIGS. 4A-F, 4H, 4I, 4L). Scale bar: 50 μm (a). AA, alopecia areata patients; NC, normal control individuals.

FIGS. 4M-O are photomicrographs of double-immunostainings with an anti-CD8 and an anti-NKG2D antibodies revealed that most CD8+ T cells co-expressed NKG2D (FIG. 4M, FIG. 4N, and FIG. 4O).

FIG. 4P is a bar graph that summarizes immunohistochemical in situ evidence of ULBP3 in human hair follicles compared between normal and lesional AA skin. Compared with control skin, immunohistology showed a significantly increased number of ULBP3+ cells in the dermis and the dermal sheath (CTS). In addition, positive cells were also up-regulated parafollicular around the hair bulb in AA samples.

FIG. 5 is a schematic showing the Confounding analysis is used to infer relationships between associated SNPs. An example is presented in FIG. 5A, in which two SNPs show significant association to a trait (in red). Directed acyclic graphs (DAGs) illustrate two alternative causal models that may underlie the observed data. In FIG. 5B, the effect observed for SNP₂ is explained entirely by the association of SNP₁ and the disease so that while OR_{SNP2}≠1, OR_{SNP2|SNP1}=1. In FIG. 5C, the effect of SNP₂ is independent of the effect of SNP₁ and conditioning on SNP₁ will not alter the OR of SNP₂ (OR_{SNP2|SNP1}≠1).

FIG. 6 are photomicrographs showing that PTGER4, STX17, and PRDX5 are expressed in human hair follicles. In FIGS. 6A-C, PTGER4 is predominantly expressed in Henle's (He) layer of the inner root sheath (IRS) of human HF. The localization of PTGER4 was confirmed by double-immunolabeling with K74 protein which is specifically expressed in Huxley's layer (Hu) of the IRS (FIGS. 6B-C). In FIGS. 6D-F, STX17 is expressed in hair shaft and IRS of human HF whose expression overlaps with K31 protein in the hair shaft cortex (HSCx). In FIGS. 6G-I, PRDX5 shows a similar expression pattern with STX17. Right panels are merged images and cells were counterstained with DAPI (FIGS. 6C, 6F, 6I). Scale bars: 100 μm.

FIG. 7 depicts mRNA expression levels of AA related genes in scalp and whole blood cells (WBC). Relative transcripts levels of AA associated genes were quantified using (FIG. 7A) quantitative PCR and (FIG. 7B) real time PCR in human scalp and whole blood sample. Elevated ULBP3 levels were observed in the scalp, IKZF4 and PTGER4 in WBC whereas PRDX5 and PTGER4 exhibited comparable expression in both. GAPDH was used as a normalization control. IL2RA and KRT15 were used as positive controls for WBC and scalp respectively.

FIG. 8 is a graph showing that immune response genes are vulnerable to positive selection, which increases allele frequencies, thus making this class of genes amenable to detection with GWAS (upper arrow). The lower arrow indicates the ‘gray zone’ of significance (5×10⁻⁷>p>0.01) for hair gene.

FIG. 9 is a graph showing the results from the linkage analyses of 471 GWAS genes, finding that 121 genes fell into regions for linkage (1<LOD<4). Results are shown for chromosome 12.

FIG. 10 is a graph showing genotyping of a small subset of patients with severe disease (AU) from the GWAS cohort at the DRB1 locus.

FIG. 11 shows the upregulation of NKG2DL expression in the unaffected HF of AA patients. Biopsies from both lesional and unaffected scalp were obtained from patients with AA and psoriasis. ULBP3 expression in isolated HF was examined by immunofluorescence.

FIGS. 12A-B shows the upregulation of HF NKG2DL in AA. FIG. 12A. qRT-PCR analysis of the expression of MICA & ULBP1-6 in normal skin and three lesional AA skin biopsies. FIG. 12B. IF microscopy of MICA & ULBP1-6 in AA and control HFs.

FIG. 13. FIG. 13A. Lysis of TNF-primed dermal sheath (DS) cells by lymphokine activated killer cells (LAKs) in vitro requires NKG2D. C3H/HeJ DS celles were treated with or without TNF for three days and then incubated with IL-2 induced LAKs in the presence of absence of neutralizing anti-NKG2D (CX5) antibody (* p<0.05). FIG. 13B. CX5 purified from hybridoma media by affinity chromatography and analyzed by SDS-PAGE.

FIG. 14 is a gel that shows and RNA analysis of ULBPs.

FIG. 15 are fluorescence photomicrographs showing in situ results of ULBP3 in normal HF(a) and HF from two AA patients (b and c).

FIG. 16 are fluorescence photomicrographs showing PRDX5 staining in normal HF.

FIG. 17 is a bar graph showing activation of the ULBP6 promoter by NF-κB pathway components. HEK293 cells were co-transfected with luciferase reporter constructs driven by tandem kB sites, or either ULBP3 or ULBP6 promoters and NF-κB p65, the MyD88 adaptor protein or NF-κB activating kinase IKKβ.

FIG. 18 is a bar graph and fluorescence photomicrographs. The bar graph (TOP) shows upregulation of NKG2DL mRNA in lesional AA skin. qRT-PCR analysis of the expression of MICA, ULBP3 and ULBP6 in normal skin and lesional AA skin biopsies. IF microscopy (BOTTOM) is shown for ULBP3 detection in AA and psoriatic skin. Lesional AA and psoriatic hair follicles and remission AA hair follicles (12 years) or non-lesional and lesional psoriatic or remission AA epidermis were stained using anti-CD3, anti-ULBP3 and with DAPI.

FIG. 19 shows CTLA4 isoforms schematic structure and their expression in human T cells. Two new CTLA4 isoforms: Li-CTLA4, ¼CTLA4 were found in human. FIG. 19A. liCTLA4 lacks exon2 which encodes the IgV-like domain that binds B7-1 (CD80) and B7-2 (CD86) ligands on antigen-presenting cells; sCTLA4 lacks exons encoding transmembrane domain and ¼CTLA4 lacks both exons 2 and 3. FIG. 19B. RT-PCR was performed in total RNA isolated from human spleen T cells. Two sets of primers were used, set 1 forward primer is specific for liCTLA4 by spanning the boundary of exon1 and 3, while set 2 is common for all the 4 isoforms by targeting exon1 and exon 4. Isoforms were confirmed by purifying the gel and subsequent sequencing: FIG. 19C. Sequence of liCTLA4 spanning exon1 and 3; FIG. 19D. Sequence of ¼CTLA4 crossing exon1 and 4.

FIG. 20 shows CTL4A expression in time-course stimulated human total blood T cells. Human total blood T cells were stimulated using CD3+CD28 Ab, cells were harvested at 0, 2, 6, 24, 50, 72, and 96 hr after Stimulation. Total RNA was extracted and RT-PCR was performed using either isoform-specific primer (Li-CTLA4) or common primer (for the rest isoforms). Beta-actin was chosen as endogenous control. CTLA4 isoforms are expressed in unstimulated T cells. After stimulation, Li-CTLA4 showed similar/stable expression after 24 hr; S-CTLA4 expression disappeared; F-CTLA4 expression is higher given longer stimulation time. Although ¼CTLA4 is expressed in unstimulated PBMC based on other experiment, it did not present here due to primer competition, it will be redone for this experiment using isoform specific primer.

FIGS. 21A-D are bar graphs of SNP rs3087243 A/G associated with Li, ¼-CTLA4 expression in total blood T cells from T1D patients. SNP rs3087243 (+6230A/G) was reported to strongly associated with autoimmune diseases, including AA and T1D. It was also shown to affect levels of soluble CTLA4—risk allele G carriers had lower expression of sCTLA4. Here, q-PCR using isoform specific primers was performed to examine CTLA4 4 expression in total blood T cells from 10 T1D patients. For each isoform, expression level was normalized to GAPDH and the relative expression level was calculated using ddCt method. Genotype data was got by direct sequencing. T1D-risk allele (G) was highlighted in red. It was found that risk allele G carriers had lower expression of Li-CTLA4 and ¼ CTLA4.

FIGS. 22A-B are graphs showing CTLA4 expression in human PBMC (AA vs. control, T1D vs. control). Q-PCR was performed using specific primers to check CTLA4 isoform expression in human PBMC. Difference was compared between 12 AA patients and 14 normal control (N=14) (upper panel), as well as 14 T1D patients and 14 normal control groups (lower panel). No difference was observed between AA vs. controls, however, Li-, ¼, and F-CTLA4 showed higher expression in T1D patients compared to controls. All expression data was normalized to GAPDH.

FIG. 23 is a gel that shows CTLA4 expression in mouse blood. Isoform-specific primers for CTLA4 were designed to check the CTLA4 expression pattern in mouse blood. RT-PCR showed all the four isoforms are expressed in mouse blood.

FIG. 24 comprises bar graphs showing higher CTLA4 expression in CTLA4-IgG treated (at 4 week) mouse. To check if exdogenous CTLA4 has any effect on preventing the development of AA, and to check if the endogenous CTLA4 expression is influenced by this process, a prevention trial was done on 3 groups of grafted mosue: Sham group (n=2) received no treatment, PBS group (n=3) only received PBS, while CTLA4-IgG group was administrated with CTLA4-IgG (n=3). Blood was extracted at 0 w, 2 w, 4 w, and 6 w. Q-PCR using isoform-specific primers was performed to check the CTLA4 expression. Gapdh was used as normalized gene. No difference in CTLA4 expression was observed among the 3 groups at 0 and 2 week. However, significantly increased expression for all the isoforms was observed in CTLA4-IgG treated group at week 4 compared to the PBS treated control group. CTLA4 expression is increased during the trial process (at least before week 4).

FIG. 25 comprises bar graphs showing higher CTLA4 expression in CTLA4-IgG treated (at 4 week) mouse. To check if exdogenous CTLA4 has any effect on preventing the development of AA, and to check if the endogenous CTLA4 expression is influenced by this process, we did a prevention trial on 3 groups of grafted mosue: Sham group (n=2) received no treatment, PBS group (n=3) only received PBS, while CTLA4-IgG group was administrated with CTLA4-IgG (n=3). Blood was extracted at 0 w, 2 w, 4 w, and 6 w. Q-PCR using isoform-specific primers was performed to check the CTLA4 expression. Gapdh was used as normalized gene. No difference in CTLA4 expression was observed among the 3 groups at 0 and 2 week. However, significantly increased expression for all the isoforms was observed in CTLA4-IgG treated group at week 4 compared to the PBS treated control group. CTLA4 expression is increased during the tial process (at least before week 4).

FIG. 26 shows Hair Follicle Expression of NKG2D ligands under inflammatory conditions. Human vibrissae follicles were micro-dissected and organ cultured for 2 days in presence of proinflammatory cytokines—IFNγ, TLR ligands—LPS or dI:dC and TNFα. Immunofluorescence staining of the human follicles for NKG2D Ligands—MICA, ULBP3 and Pan NKG2DL showed higher expression in the dermal sheath compartment of the hair follicle suggesting responsiveness to inflammatory mediators.

FIG. 27 shows elevated NKG2D ligand expression in Alopecia Areata affected skin. Quantitative realtime PCR was carried out to assess the mRNA expression levels of NKG2D ligands MICA, Ulbp3 and Ulbp6 in the affected scalp skin of alopecia areata patients compared to normal control skin. The expression levels were elevated in the affected skin compared to control skin (N=3).

FIG. 28 shows transcript expression of ULBP3 from previous microarray studies on autoimmune disorders (NIH-Gene Expression Omnibus). Data for ULBP3 expression data was derived from gene expression repository at NIH (GEO). Elevated expression of ULBP3 transcript is associated with autoimmune disorders such as psoriasis, scleroderma rheumatoid arthritis and ulcerative colitis. Elevation of ULBP3 expression is also observed in atopic disease—Asthma and contact dermatitis. This corroborates with several studies in literature which support the elevated expression of NKG2D ligands in autoimmune disorders.

FIG. 29 shows genotoxic stress (DNA damage inducing) causes transient negative regulation of NKG2D ligand promoter activity. Cloning of 5′ upstream 3 kb promoter region of MICA, ULBP3 and ULBP6 was carried out in the pGL3 Luciferase reporter vector. The effect of genotoxic Stress on ULBP promoter activity was assessed using HEK293T cells which were transfected with these reporter constructs. The transfected cells were subjected to 60 min of heat shock treatment at 42° C. followed by a recovery period of 3 h and 16 h. Cells were also subjected to 300 J/m² of UVB exposure followed by a similar 3 h and 16 h recovery. Interestingly a reduction in the promoter induced transcription of all the three NKG2D ligands—MICA, ULBP3 and ULBP6 was observed as assessed by the luciferase activity. The reduction was abrogated by 16 h after treatment indicating the transient nature of the negative regulation. On similar lines, reduction in the promoter activity post UVB treatment was also observed after 3 h of treatment. The promoter region of both ULBP3 and ULBP6 contain several heat shock factor binding elements which are induced after heatshock or UV treatment.

FIG. 30 shows the effect of heat shock on deletion constructs of ULBP3 promoter. Deletion constructs were generated to assess the role of HSE in the regulation of ULBP3 expression. The 3 kb promoter region contains several heat shock binding elements, to which heat shock factors bind and regulate expression in both positive and negative fashion. ULBP3 promoter shows HSE mediated negative regulation of expression when subjected to heat shock.

FIG. 31 shows the regulation of NKG2D ligand promoter activity by stress hormones. HEK293T cells transfected with ULBP3 promoter construct were subjected to stress hormones substance P and corticosterone for 16 h, an upregulation in the luciferase expression was observed for ULBP3 promoter (FIG. 31A). Primary fibroblasts (FIG. 31B) as well as dermal sheath (DS) cells (FIG. 31C) were transfected with NKG2D ligands MICA, ULBP3 and ULBP6 and were given 16 h treatment of stress hormones—corticotrophin releasing hormone (CRH), substance P(SP), and corticosteroids. Fibroblasts showed an upregulation of ULBP3 with CRH, SP and corticosteroid treatment whereas upregulation was observed with CRH and corticosteroid treatment in DS cells.

FIG. 32 shows the effect of inflammatory cytokines on NKG2D ligand promoter activity in Dermal Sheath cells. To assess the role of inflammatory cytokines on ULBP promoter activity, dermal sheath cells were transfected with ULBP 3′ 5-kb promoter luciferase reporter construct. A significant elevation in the promoter activity was observed in ULBP3 following an 8 hr IFNγ treatment. Dermal sheath cells transfected with ULBP6 constructs showed a dramatic upregulation with TNFa treatment. Similar effects were observed with 293 T cells. The 3′ promoter region of ULBP6 contains 3 NfκB binding sites at positions: −2940 bp, −2235 bp and −1670 bp with respect to transcriptional start site. Deletion constructs were generated omitting the NfκB binding sites and promoter activity was assessed. −2940 NFkb sites seem to contribute significantly in the TNFa induced upregulation of the ULBP6 expression.

FIG. 33 shows the effect of TNFα on deletion constructs of ULBP6 promoter in 293T Cells.

FIG. 34. shows NKG2D ligand transcript tegulation via 3′UTR under Stress Conditions. In addition to 5′ promoter region we also assessed the effect of stress on the mRNA stability. The 3′UTR region of Ulbp3 and Ulbp6 was cloned under the psiCheck2 luciferase reporter construct and transfected 293T cells with the constructs. The cells were subjected to heat shock, TNFa, IL-2 and IFNg treatment. Greater mRNA stability was observed with heatshock and TNFa treatment for ULBP3 and ULBP6. The 3′ UTR region is subject to regulation by micro RNAs. Cellular stress is associated with changes in the microRNA regulation of the genome. HEK 293T cells were co transfected with mir124, one of the predicted microRNAs binding the 3′UTR of both ULBP3 and ULBP6. RT PCR for the predicted microRNAs common to both ULBP3 and ULBP6. Cotransfection with the microRNA constructs and assay luciferase activity.

FIG. 35 shows the co-culture of target cells over expressing NKG2D ligands with NK Cells. Cloning of open reading frame of MICA, ULBP3 and ULBP6 was carried out in the pCXN1 vector and expression of ULBP3 and ULBP6 was assessed using immunofluorescence in cells transfected with the over expression vector. Primarily membrane bound and some cytoplasic expression was observed indicating membrane targeting of the expressed protein. Hek 293 T cells and primary cultures of fibroblasts as well as dermal sheath cells were transfected with the overexpression vectors for ULBP3, ULBP6 and MICA. The cells were further incubated with human natural killer cell line NK92MI to assess the differential cytotoxic response when NKG2D ligands are over expressed. Elevated cytotoxicty as assessed by elevated LDH release into the culture media was observed. The lysed target cells stained with propidium iodide shown in red and are distinguishable from live dye stained NK cell effectors (green). A greater number of propidium iodide staining cells were observed in the NKG2D ligand over-expressing dermal sheath cells.

FIG. 36 shows co-culture of human hair follicles with LAK cells. Human vibrissae follicles were micro-dissected and organ cultured for 2 days in presence of proinflammatory cytokines—IFNγ, LPS and TNFα. Individual follicles were subsequently incubated with green CFSE labeled LAK cells overnight to assess immune interaction. Elevated accumulation of LAK cells was observed on treated follicles indicating an up-regulation of NKG2D ligands on follicular surface. Mediation of cytotoxic response is in part carried out by induction of NKG2D Ligands which interact with NKG2D receptor bearing lymphocytes such as NK cells, Tc-cells, γδ T-cells. Induction of catagen in the ex vivo cultured hair follicles in presence of TNFα and IFNγ was also observed.

FIG. 37 shows a human cytotoxicity assay. Primary cultured dermal sheath cells derived from human skin were given a combined IFNγ/LPS and TNFα treatment for 3 days. Differential cytotoxic response of matching LAK cells derived from blood lymphocytes was assessed using LDH release assay. Increased cytotoxic response in treated cells was observed which is reduced in presence of NKG2D blocking antibody indicating the role of NKG2D ligand in mediating cytotoxic response. Similar assessment of cytotoxic response in primary cultured human keratinocytes treated with combined IFNγ/LPS and IFNγ/polydI:dC and TNFα also shows NKG2D receptor-ligand mediated dependence of LAK killing assay

FIG. 38 are fluorescent micrographs showing Expression of MICA, ULBP3 and ULBP6 in AA skin.

FIG. 39 are graphs showing NKG2D Ligand Transcript Expression in Alopecia Areata Skin. P FIG. 40. is a bar graph showing the effect of inflammatory cytokines on deletion constructs of ULBP promoters in 293T cells.

FIG. 41 is a bar graph showing the effect of stress hormones on NKG2D ligand promoter activity in dermal sheath cells.

FIG. 42 is a bar graph that shows the outermost mesodermal cellular layer of the hair follicles—dermal sheath (DS) cells were derived and primary cultured from micro-dissected human hair follicles. DS cells were treated with IFNγ for 24 h and the transcript levels of NKG2DLs—were assessed by real-time qPCR (N=4). An upregulation of message levels of NKG2DLs ULBP3 and MICA was observed.

FIG. 43 shows fluorescent photomicrographs. Human vibrissae follicles were micro-dissected and organ cultured for 2 days in presence of proinflammatory cytokine—IFNγ and TLR ligands—LPS and polydI:dC. Immunofluorescence staining of the follicles for NKG2D Ligands—MICA, ULBP3 and Pan NKG2DL shows higher expression in the DS compartment of the hair follicle indicating responsiveness to inflammatory mediators—IFNγ, LPS and poly dI:dC.

FIG. 44 shows photomicrographs. The efficacy of these inflammatory mediators in inducing NKG2DLs in mice was further determined by intra-dermal injections of IFNγ, LPS and IFNγ/LPS in combination in skin reinitiated for anagen phase by hair plucking. Staining of the skin, 24 hour post-treatment, showed a higher expression of Pan NKG2DL and Rae1 expression in the follicles.

FIG. 45 shows photomicrographs. Human and C3H/HeJ mice vibrissae follicles were micro-dissected and organ cultured for 2 days in presence of proinflammatory cytokine—IFNγ and TLR ligand—LPS. Individual follicles were subsequently incubated with green CFSE labeled LAK cells overnight to assess immune interaction. Elevated accumulation of LAK cells was observed on treated follicles indicating an up-regulation of interacting ligands on follicular surface. Interestingly, follicles derived from alopecic mice also showed enhanced LAK cell recruitment. Mediation of cytotoxic response is in part carried out by induction of NKG2D ligands which interact with NKG2D receptor bearing lymphocytes such as NK cells, Tc-cells, γδ T-cells.

FIG. 46 shows bar graphs. Primary cultured dermal sheath and dermal papilla cells derived from C57BL/6 mice were given a combined IFNγ and LPS treatment for 3 days. Differential cytotoxic response of match LAK cells derived from C57BL/6 lymph nodes was assessed using LDH release assay. Increased cytotoxic response in treated cells was observed which diminished in presence of NKG2D blocking antibody, thus indicating the role of NKG2D ligands in mediating cytotoxic response. Similar assessment of cytotoxic response in primary cultured human keratinocytes treated with IFNγ and polydI:dC also shows NKG2D receptor-ligand interaction mediated dependence of LAK cell cytotoxicity.

FIG. 47 shows fluorescent photomicrographs. p65 NFκB subunit KO under skin specific keratin14 basal cell component driver mice was generated. The p65 k14 cKO mice were treated with IFNγ, LPS and IFNγ/LPS intradermally for 24 h. The KO mice skin display reduced NKG2D ligands expression compared to litter mate controls in the epidermis and hair follicles as shown by pan-NKG2DL immunofluorescence staining, indicating an NFκB dependence on induction of NKG2DLs.

### DETAILED DESCRIPTION OF THE INVENTION

The invention provides for a group of genes that can be used to define susceptibility to Alopecia Areata (AA), a common autoimmune form of hair loss, where at least 8 loci have been defined, each containing several SNPS, that can be used to define such susceptibility.

There are several aspects to this invention. In one embodiment, the invention provides for a therapy that is directed against any and/or all of the genes of the group. In another embodiment, a predictive DNA-based test is used determine the likelihood and/or severity of a hair-loss disorder, such as AA.

Overview of the Integument and Hair Cells

The integument (or skin) is the largest organ of the body and is a highly complex organ covering the external surface of the body. It merges, at various body openings, with the mucous membranes of the alimentary and other canals. The integument performs a number of essential functions such as maintaining a constant internal environment via regulating body temperature and water loss; excretion by the sweat glands; but predominantly acts as a protective barrier against the action of physical, chemical and biologic agents on deeper tissues. Skin is elastic and except for a few areas such as the soles, palms, and ears, it is loosely attached to the underlying tissue. It also varies in thickness from 0.5 mm (0.02 inches) on the eyelids (“thin skin”) to 4 mm (0.17 inches) or more on the palms and soles (“thick skin”) (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The skin is composed of two layers: a) the epidermis and b) the dermis. The epidermis is the outer layer, which is comparatively thin (0.1 mm). It is several cells thick and is composed of 5 layers: the stratum germinativum, stratum spinosum, stratum granulosum, stratum lucidum (which is limited to thick skin), and the stratum corneum. The outermost epidermal layer (the stratum corneum) consists of dead cells that are constantly shed from the surface and replaced from below by a single, basal layer of cells, called the stratum germinativum. The epidermis is composed predominantly of keratinocytes, which make up over 95% of the cell population. Keratinocytes of the basal layer (stratum germinativum) are constantly dividing, and daughter cells subsequently move upwards and outwards, where they undergo a period of differentiation, and are eventually sloughed off from the surface. The remaining cell population of the epidermis includes dendritic cells such as Langerhans cells and melanocytes. The epidermis is essentially cellular and non-vascular, containing little extracellular matrix except for the layer of collagen and other proteins beneath the basal layer of keratinocytes (Ross M H, Histology: A text and atlas, 3^{rd} edition, Williams and Wilkins, 1995: Chapter 14; Burkitt H G, et al, Wheater's Functional Histology. 3^{rd} Edition, Churchill Livingstone, 1996: Chapter 9).

The dermis is the inner layer of the skin and is composed of a network of collagenous extracellular material, blood vessels, nerves, and elastic fibers. Within the dermis are hair follicles with their associated sebaceous glands (collectively known as the pilosebaceous unit) and sweat glands. The interface between the epidermis and the dermis is extremely irregular and uneven, except in thin skin. Beneath the basal epidermal cells along the epidermal-dermal interface, the specialized extracellular matrix is organized into a distinct structure called the basement membrane (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The mammalian hair fiber is composed of keratinized cells and develops from the hair follicle. The hair follicle is a peg of tissue derived from a downgrowth of the epidermis, which lies immediately underneath the skin's surface. The distal part of the hair follicle is in direct continuation with the external, cutaneous epidermis. Although a small structure, the hair follicle comprises a highly organized system of recognizably different layers arranged in concentric series. Active hair follicles extend down through the dermis, the hypodermis (which is a loose layer of connective tissue), and into the fat or adipose layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

At the base of an active hair follicle lies the hair bulb. The bulb consists of a body of dermal cells, known as the dermal papilla, contained in an inverted cup of epidermal cells known as the epidermal matrix. Irrespective of follicle type, the germinative epidermal cells at the very base of this epidermal matrix produce the hair fiber, together with several supportive epidermal layers. The lowermost dermal sheath is contiguous with the papilla basal stalk, from where the sheath curves externally around all of the hair matrix epidermal layers as a thin covering of tissue. The lowermost portion of the dermal sheath then continues as a sleeve or tube for the length of the follicle (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

Developing skin appendages, such as hair and feather follicles, rely on the interaction between the epidermis and the dermis, the two layers of the skin. In embryonic development, a sequential exchange of information between these two layers supports a complex series of morphogenetic processes, which results in the formation of adult follicle structures. However, in contrast to general skin dermal and epidermal cells, certain hair follicle cell populations, following maturity, retain their embryonic-type interactive, inductive, and biosynthetic behaviors. These properties can be derived from the very dynamic nature of the cyclical productive follicle, wherein repeated tissue remodeling necessitates a high level of dermal-epidermal interactive communication, which is vital for embryonic development and would be desirable in other forms of tissue reconstruction.

The hair fiber is produced at the base of an active follicle at a very rapid rate. For example, follicles produce hair fibers at a rate 0.4 mm per day in the human scalp and up to 1.5 mm per day in the rat vibrissa or whiskers, which means that cell proliferation in the follicle epidermis ranks amongst the fastest in adult tissues (Malkinson F D and J T Kearn, Int J Dermatol 1978, 17:536-551). Hair grows in cycles. The anagen phase is the growth phase, wherein up to 90% of the hair follicles said to be in anagen; catagen is the involuting or regressing phase which accounts for about 1-2% of the hair follicles; and telogen is the resting or quiescent phase of the cycle, which accounts for about 10-14% of the hair follicles. The cycle's length varies on different parts of the body.

Hair follicle formation and cycling is controlled by a balance of inhibitory and stimulatory signals. The signaling cues are potentiated by growth factors that are members of the TGFβ-BMP family. A prominent antagonist of the members of the TGFβ-BMP family is follistatin. Follistatin is a secreted protein that inhibits the action of various BMPs (such as BMP-2, -4, -7, and -11) and activins by binding to said proteins, and purportedly plays a role in the development of the hair follicle (Nakamura M, et al., FASEB J, 2003, 17(3):497-9; Patel K Intl J Biochem Cell Bio, 1998, 30:1087-93; Ueno N, et al., PNAS, 1987, 84:8282-86; Nakamura T, et al., Nature, 1990, 247:836-8; Iemura S, et al., PNAS, 1998, 77:649-52; Fainsod A, et al., Mech Dev, 1997, 63:39-50; Gamer L W, et al., Dev Biol, 1999, 208:222-32).

The deeply embedded end bulb, where local dermal-epidermal interactions drive active fiber growth, is the signaling center of the hair follicle comprising a cluster of mesencgymal cells, called the dermal papilla (DP). This same region is also central to the tissue remodeling and developmental changes involved in the hair fiber's or appendage's precise alternation between growth and regression phases. The DP, a key player in these activities, appears to orchestrate the complex program of differentiation that characterizes hair fiber formation from the primitive germinative epidermal cell source (Oliver R F, J Soc Cosmet Chem, 1971, 22:741-755; Oliver R F and C A Jahoda, Biology of Wool and Hair (eds Roger et al.), 1971, Cambridge University Press:51-67; Reynolds A J and C A Jahoda, Development, 1992, 115:587-593; Reynolds A J, et al., J Invest Dermatol, 1993, 101:634-38).

The lowermost dermal sheath (DS) arises below the basal stalk of the papilla, from where it curves outwards and upwards. This dermal sheath then externally encases the layers of the epidermal hair matrix as a thin layer of tissue and continues upward for the length of the follicle. The epidermally-derived outer root sheath (ORS) also continues for the length of the follicle, which lies immediately internal to the dermal sheath in between the two layers, and forms a specialized basement membrane termed the glassy membrane. The outer root sheath constitutes little more than an epidermal monolayer in the lower follicle, but becomes increasingly thickened as it approaches the surface. The inner root sheath (IRS) forms a mold for the developing hair shaft. It comprises three parts: the Henley layer, the Huxley layer, and the cuticle, with the cuticle being the innermost portion that touches the hair shaft. The IRS cuticle layer is a single cell thick and is located adjacent to the hair fiber. It closely interdigitates with the hair fiber cuticle layer. The Huxley layer can comprise up to four cell layers. The IRS Henley layer is the single cell layer that runs adjacent to the ORS layer (Ross M H, Histology: A text and atlas, 3^{rd} edition, Williams and Wilkins, 1995: Chapter 14; Burkitt H G, et al, Wheater's Functional Histology. 3^{rd} Edition, Churchill Livingstone, 1996: Chapter 9).

Alopecia Areata

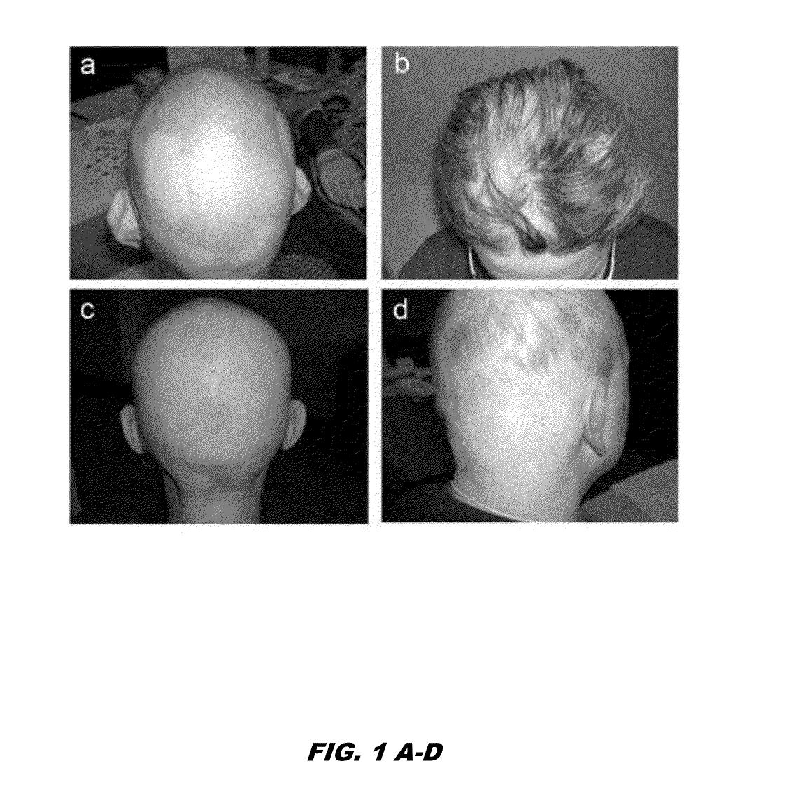

Alopecia areata (AA) is one of the most prevalent autoimmune diseases, affecting approximately 4.6 million people in the US alone, including males and females across all ethnic groups, with a lifetime risk of 1.7%.^{A1} In AA, autoimmunity develops against the hair follicle, resulting in non-scarring hair loss that may begin as patches, which can coalesce and progress to cover the entire scalp (alopecia totalis, AT) or eventually the entire body (alopecia universalis, AU) (FIG. 1). AA was first described by Cornelius Celsus in 30 A.D., using the term “ophiasis”, which means “snake”, due to the sinuous path of hair loss as it spread slowly across the scalp. Hippocrates first used the Greek word ‘alopekia’ (fox mange), the modern day term “alopecia areata” was first used by Sauvages in his Nosologica Medica, published in 1760 in Lyons, France.

Curiously, AA affects pigmented hair follicles in the anagen (growth) phase of the hair cycle, and when the hair regrows in patches of AA, it frequently grows back white or colorless. The phenomenon of ‘sudden whitening of the hair’ is therefore ascribed to AA with an acute onset, and has been documented throughout history as having affected several prominent individuals at times of profound grief, stress or fear.^{A2} Examples include Shahjahan, who upon the death of his wife in 1631 experienced acute whitening of his hair, and in his grief built the Taj Mahal in her honor. Sir Thomas More, author of Utopia, who on the eve of his execution in 1535 was said to have become ‘white in both beard and hair’. The sudden whitening of the hair is believed to result from an acute attack upon the pigmented hair follicles, leaving behind the white hairs unscathed.

Several clinical aspects of AA remain unexplained but may hold important clues toward understanding pathogenesis. AA attacks hairs only around the base of the hair follicles, which are surrounded by dense clusters of lymphocytes, resulting in the pathognomic ‘swarm of bees’ appearance on histology. Based on these observations, and without being bound by theory, a signal(s) in the pigmented, anagen hair follicle is emitted invoking an acute or chronic immune response against the lower end of the hair follicle, leading to hair cycle perturbation, acute hair shedding, hair shaft anomalies, and hair breakage. Despite these perturbations in the hair follicle, there is no permanent organ destruction and the possibility of hair regrowth remains if immune privilege can be restored.

Throughout history, AA has been considered at times to be a neurological disease brought on by stress or anxiety, or as a result of an infectious agent, or even hormonal dysfunction. The concept of a genetically-determined autoimmune mechanism as the basis for AA emerged during the 20^{th} century from multiple lines of evidence. AA hair follicles exhibit an immune infiltrate with activated Th, Tc and NK cells^{A3,A4} and there is a shift from a suppressive (Th2) to an autoimmune (Th1) cytokine response. The humanized model of AA, which involves transfer of AA patient scalp onto immune-deficient SCID mice illustrates the autoimmune nature of the disease, since transfer of donor T-cells causes hair loss only when co-cultured with hair follicle or human melanoma homogenate.^{A5,A6} Regulatory T cells which serve to maintain immune tolerance are observed in lower numbers in AA tissue,^{A7} and transfer of these cells to C3H/HeJ mice leads to resistance to AA.^{A8} Although AA has long been considered exclusively as a T-cell mediated disease, in recent years, an additional mechanism of disease has been discussed. The hair follicle is defined as one of a select few immune privileged sites in the body, characterized by the presence of extracellular matrix barriers to impede immune cell trafficking, lack of antigen presenting cells, and inhibition of NK cell activity via the local production of immunosuppressive factors and reduced levels of MHC class I expression.^{A9} Thus, the notion of a ‘collapse of immune privilege’ has also been invoked as part of the mechanism by which AA may arise. Support for a genetic basis for AA comes from multiple lines of evidence, including the observed heritability in first degree relatives,^{A10, A11} twin studies,^{A12} and most recently, from the results of our family-based linkage studies.^{A13}

Hair Loss Disorder Gene Cohort (HLDGC)

This invention provides for the discovery that a number human genes have, for the first time, been identified as a cohort of genes involved in hair loss disorders. These genes were identified as having particular single-nucleotide polymorphisms where the presence of such particular polymorphism was correlated with the presence of a hair loss disorder in a subject. These genes, now that they have been identified, can be used for a variety of useful methods; for example, they can be used to determine whether a subject has susceptibility to Alopecia Areata (AA). The genes identified as part of this hair loss disorder gene cohort or group (i.e., “HLDGC genes”) include CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, and PTPN2. In one embodiment, a HLA Region residing gene is selected from the group consisting of a gene of the HLA Class I Region, a gene of the HLA Class II Region, PTPN22, and AIRE. In one embodiment, the HLA Class I Region gene is HLA-A, HLA-B, HLA-C, HLA-G, HLA-DQB1, HLA-DRB1, MICA, MICB, or NOTCH4. In one embodiment, the HLA Class II Region gene is HLA-DOB, HLA-DQA1, HLA-DQA2, HLA-DQB2, TAP2, or HLA-DRA.

In one embodiment, the invention provides methods to diagnose a hair loss disorder or methods to treat a hair loss disorder comprising use of nucleic acids or proteins encoded by nucleic acids of the following HLDGC genes here discovered to be associated with alopecia areata: CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, and PTPN2. For example, a HLDGC protein can be the human CTLA-4 protein (e.g., having the amino acid sequence shown in SEQ ID NO: 1); the human IL-2 protein (e.g., having the amino acid sequence shown in SEQ ID NO: 3); the human IL-2RA/CD25 protein (e.g., having the amino acid sequence shown in SEQ ID NO: 5); the human IKZF4 protein (e.g., having the amino acid sequence shown in SEQ ID NO: 7); the human PTGER4 protein (e.g., having the amino acid sequence shown in SEQ ID NO: 9); the human PRDX5 protein (e.g., having the amino acid sequence shown in SEQ ID NO: 11); the human STX17 protein (e.g., having the amino acid sequence shown in SEQ ID NO: 13); the human NKG2D protein (e.g., having the amino acid sequence shown in SEQ ID NO: 15); the human ULBP6 protein (e.g., having the amino acid sequence shown in SEQ ID NO: 17); the human ULBP3 protein (e.g., having the amino acid sequence shown in SEQ ID NO: 19); the human IL-21 protein (e.g., having the amino acid sequence shown in SEQ ID NO: 21); or a human HLA Class II Region protein, such as HLA-DQA2 (e.g., having the amino acid sequence shown in SEQ ID NO: 23). In one embodiment, a HLA Region residing gene is selected from the group consisting of a gene of the HLA Class I Region, a gene of the HLA Class II Region, PTPN22, and AIRE. In one embodiment, the HLA Class I Region gene is HLA-A, HLA-B, HLA-C, HLA-DQB1, HLA-DRB1, MICA, and NOTCH4. In one embodiment, the HLA Class II Region gene is HLA-DOB, HLA-DQA1, HLA-DQA2, HLA-DQB2, and HLA-DRA.

In some embodiments, the invention encompasses methods for using HLDGC proteins encoded by a nucleic acid (including, for example, genomic DNA, complementary DNA (cDNA), synthetic DNA, as well as any form of corresponding RNA). For example, a HLDGC protein can be encoded by a recombinant nucleic acid of a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In one embodiment, a HLA Region residing gene is selected from the group consisting of a gene of the HLA Class I Region, a gene of the HLA Class II Region, PTPN22, and AIRE. In one embodiment, the HLA Class I Region gene is HLA-A, HLA-B, HLA-C, HLA-DQB1, HLA-DRB1, MICA, MICB, HLA-G, or NOTCH4. In one embodiment, the HLA Class II Region gene is HLA-DOB, HLA-DQA1, HLA-DQA2, HLA-DQB2, TAP2, or HLA-DRA. The HLDGC proteins of the invention can be obtained from various sources and can be produced according to various techniques known in the art. For example, a nucleic acid that encodes a HLDGC protein can be obtained by screening DNA libraries, or by amplification from a natural source. A HLDGC protein can include a fragment or portion of human CTLA-4 protein, IL-2, IL-21 protein, IL-2RA/CD25 protein, IKZF4 protein, a HLA Region residing protein, PTGER4 protein, PRDX5 protein, STX17 protein, NKG2D protein, ULBP6 protein, ULBP3 protein, HDAC4 protein, CACNA2D3 protein, IL-13 protein, IL-6 protein, CHCHD3 protein, CSMD1 protein, IFNG protein, IL-26 protein, KIAA0350 (CLEC16A) protein, SOCS1 protein, ANKRD12 protein, or PTPN2 protein. The nucleic acids encoding HLDGC proteins of the invention can be produced via recombinant DNA technology and such recombinant nucleic acids can be prepared by conventional techniques, including chemical synthesis, genetic engineering, enzymatic techniques, or a combination thereof. Non-limiting examples of a HLDGC protein is the polypeptide encoded by either the nucleic acid having the nucleotide sequence shown in SEQ ID NO: 2, 4, 6, 8, 10, 12, 14, 16, 18, 20, 22, or 24.

In another embodiment, the invention encompasses orthologs of a human HLDGC protein, such as CTLA-4, IL-2, IL-21, IL-2RA/CD25, IKZF4, a protein encoded by a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, and PTPN2. In one embodiment, a HLA Region residing gene is selected from the group consisting of a gene of the HLA Class I Region, a gene of the HLA Class II Region, PTPN22, and AIRE. In one embodiment, the HLA Class I Region gene is HLA-A, HLA-B, HLA-C, HLA-DQB1, HLA-DRB1, MICA, MICB, HLA-G, or NOTCH4. In one embodiment, the HLA Class II Region gene is HLA-DOB, HLA-DQA1, HLA-DQA2, HLA-DQB2, TAP2, or HLA-DRA. For example, an HLDGC protein can encompass the ortholog in mouse, rat, non-human primates, canines, goat, rabbit, porcine, bovine, chickens, feline, and horses. In one embodiment, the invention encompasses a protein encoded by a nucleic acid sequence homologous to the human nucleic acid, wherein the nucleic acid is found in a different species and wherein that homolog encodes a protein similar to a protein encoded by a HLDGC gene, such as CTLA-4, IL-2, IL-21, IL-2RA/CD25, IKZF4, a HLA Region residing protein, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, and PTPN2. In some embodiments, the invention provides methods to treat a hair loss disorder in non-human animals (i.e., treating pet mange). The method can comprise using orthologs of a human HLDGC protein or nucleic acids encoding the same.

In some embodiments, the invention encompasses use of variants of an HLDGC protein, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, and PTPN2. Such a variant can comprise a naturally-occurring variant due to allelic variations between individuals (e.g., polymorphisms), mutated alleles related to hair growth, density, or pigmentation, or alternative splicing forms.

In one embodiment, the invention encompasses methods for using a protein or polypeptide encoded by a nucleic acid sequence of a Hair Loss Disorder Gene Cohort (HLDGC) gene, such as the sequence shown in SEQ ID NO: 1, 3, 5, 7, 9, 11, 13, 15, 17, 19, 21 or 23. In another embodiment, the polypeptide can be modified, such as by glycosylations and/or acetylations and/or chemical reaction or coupling, and can contain one or several non-natural or synthetic amino acids. An example of a HLDGC polypeptide has the amino acid sequence shown in SEQ ID NO: 1, 3, 5, 7, 9, 11, 13, 15, 17, 19, 21, or 23. In certain embodiments, the invention encompasses variants of a human protein encoded by a Hair Loss Disorder Gene Cohort (HLDGC) gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, and PTPN2. Such variants can include those having at least from about 46% to about 50% identity to SEQ ID NO: 1, 3, 5, 7, 9, 11, 13, 15, 17, 19, 21, or 23, or having at least from about 50.1% to about 55% identity to SEQ ID NO: 1, 3, 5, 7, 9, 11, 13, 15, 17, 19, 21, or 23, or having at least from about 55.1% to about 60% identity to SEQ ID NO: 1, 3, 5, 7, 9, 11, 13, 15, 17, 19, 21, or 23, or having from at least about 60.1% to about 65% identity to SEQ ID NO: 1, 3, 5, 7, 9, 11, 13, 15, 17, 19, 21, or 23, or having from about 65.1% to about 70% identity to SEQ ID NO: 1, 3, 5, 7, 9, 11, 13, 15, 17, 19, 21, or 23, or having at least from about 70.1% to about 75% identity to SEQ ID NO: 1, 3, 5, 7, 9, 11, 13, 15, 17, 19, 21, or 23, or having at least from about 75.1% to about 80% identity to SEQ ID NO: 1, 3, 5, 7, 9, 11, 13, 15, 17, 19, 21, or 23, or having at least from about 80.1% to about 85% identity to SEQ ID NO: 1, 3, 5, 7, 9, 11, 13, 15, 17, 19, 21, or 23, or having at least from about 85.1% to about 90% identity to SEQ ID NO: 1, 3, 5, 7, 9, 11, 13, 15, 17, 19, 21, or 23, or having at least from about 90.1% to about 95% identity to SEQ ID NO: 1, 3, 5, 7, 9, 11, 13, 15, 17, 19, 21, or 23, or having at least from about 95.1% to about 97% identity to SEQ ID NO: 1, 3, 5, 7, 9, 11, 13, 15, 17, 19, 21, or 23, or having at least from about 97.1% to about 99% identity to SEQ ID NO: 1, 3, 5, 7, 9, 11, 13, 15, 17, 19, 21, or 23.

The polypeptide sequence of human CTLA4 is depicted in SEQ ID NO: 1. The nucleotide sequence of human CTLA4 is shown in SEQ ID NO: 2. Sequence information related to CTLA4 is accessible in public databases by GenBank Accession numbers NM_{—}005214 (for mRNA) and NP_{—}005205 (for protein).

CTLA4, also known as CD152, is a member of the immunoglobulin superfamily, which is expressed on the surface of Helper T cells. CTLA4 is similar to the T-cell costimulatory protein CD28. Both CTLA4 and CD28 molecules bind to CD80 and CD86 on antigen-presenting cells. CTLA4 transmits an inhibitory signal to T cells, while CD28 transmits a stimulatory signal. (Yamada R, Ymamoto K. Mutat Res. 2005 Jun. 3; 573(1-2):136-51; and Gough S C, Walker L S, Sansom D M. Immunol Rev. 2005 April; 204:102-150).

SEQ ID NO: 1 is the human wild type amino acid sequence corresponding to CTLA4 (residues 1-223):

| | |
|---|---|
| 1 | MACLGFQRHK AQLNLATRTW PCTLLFFLLF IPVFCKAMHV AQPAVVLASS RGIASFVCEY |
| 61 | ASPGKATEVR VTVLRQADSQ VTEVCAATYM MGNELTFLDD SICTGTSSGN QVNLTIQGLR |
| 121 | AMDTGLYICK VELMYPPPYY LGIGNGTQIY VIDPEPCPDS DFLLWILAAV SSGLFFYSFL |
| 181 | LTAVSLSKML KKRSPLTTGV YVKMPPTEPE CEKQFQPYFI PIN |

SEQ ID NO: 2 is the human wild type nucleotide sequence corresponding to CTLA4 (nucleotides 1-1988), wherein the underscored bolded “ATG” denotes the beginning of the open reading frame:

| | |
|---|---|
| 1 | gccttctgtg tgtgcacatg tgtaatacat atctgggatc aaagctatct atataaagtc |
| 61 | cttgattctg tgtgggttca aacacatttc aaagcttcag gatcctgaaa ggttttgctc |
| 121 | tacttcctga agacctgaac accgctccca taaagccatg gcttgccttg gatttcagcg |
| 181 | gcacaaggct cagctgaacc tggctaccag gacctggccc tgcactctcc tgttttttct |
| 241 | tctcttcatc cctgtcttct gcaaagcaat gcacgtggcc cagcctgctg tggtactggc |
| 301 | cagcagccga ggcatcgcca gctttgtgtg tgagtatgca tctccaggca aagccactga |
| 361 | ggtccgggtg acagtgcttc ggcaggctga cagccaggtg actgaagtct gtgcggcaac |
| 421 | ctacatgatg gggaatgagt tgaccttcct agatgattcc atctgcacgg gcacctccag |
| 481 | tggaaatcaa gtgaacctca ctatccaagg actgagggcc atggacacgg gactctacat |
| 541 | ctgcaaggtg gagctcatgt acccaccgcc atactacctg ggcataggca acggaaccca |
| 601 | gatttatgta attgatccag aaccgtgccc agattctgac ttcctcctct ggatccttgc |
| 661 | agcagttagt tcggggttgt ttttttatag ctttctcctc acagctgttt ctttgagcaa |
| 721 | aatgctaaag aaaagaagcc ctcttacaac aggggtctat gtgaaaatgc ccccaacaga |
| 781 | gccagaatgt gaaaagcaat ttcagcctta ttttattccc atcaattgag aaaccattat |
| 841 | gaagaagaga gtccatattt caatttccaa gagctgaggc aattctaact tttttgctat |
| 901 | ccagctattt ttatttgttt gtgcatttgg ggggaattca tctctcttta atataaagtt |
| 961 | ggatgcggaa cccaaattac gtgtactaca atttaaagca aaggagtaga aagacagagc |
| 1021 | tgggatgttt ctgtcacatc agctccactt tcagtgaaag catcacttgg gattaatatg |
| 1081 | gggatgcagc attatgatgt gggtcaagga attaagttag ggaatggcac agcccaaaga |
| 1141 | aggaaaaggc agggagcgag ggagaagact atattgtaca caccttatat ttacgtatga |
| 1201 | gacgtttata gccgaaatga tcttttcaag ttaaatttta tgccttttat ttcttaaaca |
| 1261 | aatgtatgat tacatcaagg cttcaaaaat actcacatgg ctatgtttta gccagtgatg |
| 1321 | ctaaaggttg tattgcatat atacatatat atatatatat atatatatat atatatatat |
| 1381 | atatatatat atatatatat tttaatttga tagtattgtg catagagcca cgtatgtttt |
| 1441 | tgtgtatttg ttaatggttt gaatataaac actatatggc agtgtctttc caccttgggt |
| 1501 | cccagggaag ttttgtggag gagctcagga cactaataca ccaggtagaa cacaaggtca |
| 1561 | tttgctaact agcttggaaa ctggatgagg tcatagcagt gcttgattgc gtggaattgt |
| 1621 | gctgagttgg tgttgacatg tgctttgggg cttttacacc agttcctttc aatggtttgc |
| 1681 | aaggaagcca cagctggtgg tatctgagtt gacttgacag aacactgtct tgaagacaat |
| 1741 | ggcttactcc aggagaccca caggtatgac cttctaggaa gctccagttc gatgggccca |
| 1801 | attcttacaa acatgtggtt aatgccatgg acagaagaag gcagcaggtg gcagaatggg |
| 1861 | gtgcatgaag gtttctgaaa attaacactg cttgtgtttt taactcaata ttttccatga |
| 1921 | aaatgcaaca acatgtataa tatttttaat taaataaaaa tctgtggtgg tcgttttaaa |
| 1981 | aaaaaaaa |

The polypeptide sequence of human IL-2 is depicted in SEQ ID NO: 3. The nucleotide sequence of human IL-2 is shown in SEQ ID NO: 4. Sequence information related to IL-2 is accessible in public databases by GenBank Accession numbers NM_{—}000586 (for mRNA) and NP_{—}000577 (for protein).

Interleukin-2 (IL-2) is a cytokine produced by the body in an immune response to a foreign agent (an antigen), such as a microbial infection. IL-2 is involved in discriminating between foreign (non-self) and self. (See Rochman Y, Spolski R, Leonard W J. Nat Rev Immunol. 2009 July; 9(7):480-90; and Overwijk W W, Schluns K S. Clin Immunol. August; 132(2):153-65).

SEQ ID NO: 3 is the human wild type amino acid sequence corresponding to IL-2 (residues 1-153):

1 MYRMQLLSCI ALSLALVTNS APTSSSTKKT QLQLEHLLLD LQMILNGINN YKNPKLTRML

61 TFKFYMPKKA TELKHLQCLE EELKPLEEVL NLAQSKNFHL RPRDLISNIN VIVLELKGSE

121 TTFMCEYADE TATIVEFLNR WITFCQSIIS TLT

SEQ ID NO: 4 is the human wild type nucleotide sequence corresponding to IL-2 (nucleotides 1-822), wherein the underscored bolded “ATG” denotes the beginning of the open reading frame:

| | |
|---|---|
| 1 | agttccctat cactctcttt aatcactact cacagtaacc tcaactcctg ccacaatgta |
| 61 | caggatgcaa ctcctgtctt gcattgcact aagtcttgca cttgtcacaa acagtgcacc |
| 121 | tacttcaagt tctacaaaga aaacacagct acaactggag catttactgc tggatttaca |
| 181 | gatgattttg aatggaatta ataattacaa gaatcccaaa ctcaccagga tgctcacatt |
| 241 | taagttttac atgcccaaga aggccacaga actgaaacat cttcagtgtc tagaagaaga |
| 301 | actcaaacct ctggaggaag tgctaaattt agctcaaagc aaaaactttc acttaagacc |
| 361 | cagggactta atcagcaata tcaacgtaat agttctggaa ctaaagggat ctgaaacaac |
| 421 | attcatgtgt gaatatgctg atgagacagc aaccattgta gaatttctga acagatggat |
| 481 | taccttttgt caaagcatca tctcaacact gacttgataa ttaagtgctt cccacttaaa |
| 541 | acatatcagg ccttctattt atttaaatat ttaaatttta tatttattgt tgaatgtatg |
| 601 | gtttgctacc tattgtaact attattctta atcttaaaac tataaatatg gatcttttat |
| 661 | gattcttttt gtaagcccta ggggctctaa aatggtttca cttatttatc ccaaaatatt |
| 721 | tattattatg ttgaatgtta aatatagtat ctatgtagat tggttagtaa aactatttaa |
| 781 | taaatttgat aaatataaaa aaaaaaaaaa aaaaaaaaaa aa |

The polypeptide sequence of human IL-2RA is depicted in SEQ ID NO: 5. The nucleotide sequence of human IL-2RA/CD25 is shown in SEQ ID NO: 6. Sequence information related to IL-2RA is accessible in public databases by GenBank Accession numbers NM_{—}000417 (for mRNA) and NP_{—}000408 (for protein).

IL-2RA, type I transmembrane protein, is the receptor for the alpha chain of Interleukin-2 (IL-2) that is present on activated T cells and activated B cells. In combination with IL-2RB and IL-2RG, it forms the heterotrimeric IL-2 receptor (Waldmann T A. J Clin Immunol. 2002 March; 22(2):51-6).

SEQ ID NO: 5 is the human wild type amino acid sequence corresponding to IL-2RA/CD25 (residues 1-272):

| | |
|---|---|
| 1 | MDSYLLMWGL LTFIMVPGCQ AELCDDDPPE IPHATFKAMA YKEGTMLNCE CKRGFRRIKS |
| 61 | GSLYMLCTGN SSHSSWDNQC QCTSSATRNT TKQVTPQPEE QKERKTTEMQ SPMQPVDQAS |
| 121 | LPGHCREPPP WENEATERIY HFVVGQMVYY QCVQGYRALH RGPAESVCKM THGKTRWTQP |
| 181 | QLICTGEMET SQFPGEEKPQ ASPEGRPESE TSCLVTTTDF QIQTEMAATM ETSIFTTEYQ |
| 241 | VAVAGCVFLL ISVLLLSGLT WQRRQRKSRR TI |

SEQ ID NO: 6 is the human wild type nucleotide sequence corresponding to IL-2RA/CD25 (nucleotides 1-2308), wherein the underscored bolded “ATG” denotes the beginning of the open reading frame:

| | |
|---|---|
| 1 | gagagactgg atggacccac aagggtgaca gcccaggcgg accgatcttc ccatcccaca |
| 61 | tcctccggcg cgatgccaaa aagaggctga cggcaactgg gccttctgca gagaaagacc |
| 121 | tccgcttcac tgccccggct ggtcccaagg gtcaggaaga tggattcata cctgctgatg |
| 181 | tggggactgc tcacgttcat catggtgcct ggctgccagg cagagctctg tgacgatgac |
| 241 | ccgccagaga tcccacacgc cacattcaaa gccatggcct acaaggaagg aaccatgttg |
| 301 | aactgtgaat gcaagagagg tttccgcaga ataaaaagcg ggtcactcta tatgctctgt |
| 361 | acaggaaact ctagccactc gtcctgggac aaccaatgtc aatgcacaag ctctgccact |
| 421 | cggaacacaa cgaaacaagt gacacctcaa cctgaagaac agaaagaaag gaaaaccaca |
| 481 | gaaatgcaaa gtccaatgca gccagtggac caagcgagcc ttccaggtca ctgcagggaa |
| 541 | cctccaccat gggaaaatga agccacagag agaatttatc atttcgtggt ggggcagatg |
| 601 | gtttattatc agtgcgtcca gggatacagg gctctacaca gaggtcctgc tgagagcgtc |
| 661 | tgcaaaatga cccacgggaa gacaaggtgg acccagcccc agctcatatg cacaggtgaa |
| 721 | atggagacca gtcagtttcc aggtgaagag aagcctcagg caagccccga aggccgtcct |
| 781 | gagagtgaga cttcctgcct cgtcacaaca acagattttc aaatacagac agaaatggct |
| 841 | gcaaccatgg agacgtccat atttacaaca gagtaccagg tagcagtggc cggctgtgtt |
| 901 | ttcctgctga tcagcgtcct cctcctgagt gggctcacct ggcagcggag acagaggaag |
| 961 | agtagaagaa caatctagaa aaccaaaaga acaagaattt cttggtaaga agccgggaac |
| 1021 | agacaacaga agtcatgaag cccaagtgaa atcaaaggtg ctaaatggtc gcccaggaga |
| 1081 | catccgttgt gcttgcctgc gttttggaag ctctgaagtc acatcacagg acacggggca |
| 1141 | gtggcaacct tgtctctatg ccagctcagt cccatcagag agcgagcgct acccacttct |
| 1201 | aaatagcaat ttcgccgttg aagaggaagg gcaaaaccac tagaactctc catcttattt |
| 1261 | tcatgtatat gtgttcatta aagcatgaat ggtatggaac tctctccacc ctatatgtag |
| 1321 | tataaagaaa agtaggttta cattcatctc attccaactt cccagttcag gagtcccaag |
| 1381 | gaaagcccca gcactaacgt aaatacacaa cacacacact ctaccctata caactggaca |
| 1441 | ttgtctgcgt ggttcctttc tcagccgctt ctgactgctg attctcccgt tcacgttgcc |
| 1501 | taataaacat ccttcaagaa ctctgggctg ctacccagaa atcattttac ccttggctca |
| 1561 | atcctctaag ctaaccccct tctactgagc cttcagtctt gaatttctaa aaaacagagg |
| 1621 | ccatggcaga ataatctttg ggtaacttca aaacggggca gccaaaccca tgaggcaatg |
| 1681 | tcaggaacag aaggatgaat gaggtcccag gcagagaatc atacttagca aagttttacc |
| 1741 | tgtgcgttac taattggcct ctttaagagt tagtttcttt gggattgcta tgaatgatac |
| 1801 | cctgaatttg gcctgcacta atttgatgtt tacaggtgga cacacaaggt gcaaatcaat |
| 1861 | gcgtacgttt cctgagaagt gtctaaaaac accaaaaagg gatccgtaca ttcaatgttt |
| 1921 | atgcaaggaa ggaaagaaag aaggaagtga agagggagaa gggatggagg tcacactggt |
| 1981 | agaacgtaac cacggaaaag agcgcatcag gcctggcacg gtggctcagg cctataaccc |
| 2041 | cagctcccta ggagaccaag gcgggagcat ctcttgaggc caggagtttg agaccagcct |
| 2101 | gggcagcata gcaagacaca tccctacaaa aaattagaaa ttggctggat gtggtggcat |
| 2161 | acgcctgtag tcctagccac tcaggaggct gaggcaggag gattgcttga gcccaggagt |
| 2221 | tcgaggctgc agtcagtcat gatggcacca ctgcactcca gcctgggcaa cagagcaaga |
| 2281 | tcctgtcttt aaggaaaaaa agacaagg |

The polypeptide sequence of human IKZF4 (IKAROS family zinc finger 4 (Eos)) is depicted in SEQ ID NO: 7. The nucleotide sequence of human IKZF4 is shown in SEQ ID NO: 8. Sequence information related to IKZF4 is accessible in public databases by GenBank Accession numbers NM_{—}022465 (for mRNA) and NP_{—}071910 (for protein).

IKZF4 is a zinc-finger protein that is a member of the Ikaros family of transcription factors. (John L B, Yoong S, Ward A C. J. Immunol. 2009 Apr. 15; 182(8):4792-9; and Perdomo J, Holmes M, Chong B, Crossley M. J Biol. Chem. 2000 Dec. 8; 275(49):38347-54).

SEQ ID NO: 7 is the human wild type amino acid sequence corresponding to IKZF4 (residues 1-585):

| | |
|---|---|
| 1 | MHTPPALPRR FQGGGRVRTP GSHRQGKDNL ERDPSGGCVP DFLPQAQDSN HFIMESLFCE |
| 61 | SSGDSSLEKE FLGAPVGPSV STPNSQHSSP SRSLSANSIK VEMYSDEESS RLLGPDERLL |
| 121 | EKDDSVIVED SLSEPLGYCD GSGPEPHSPG GIRLPNGKLK CDVCGMVCIG PNVLMVHKRS |
| 181 | HTGERPFHCN QCGASFTQKG NLLRHIKLHS GEKPFKCPFC NYACRRRDAL TGHLRTHSVS |
| 241 | SPTVGKPYKC NYCGRSYKQQ STLEEHKERC HNYLQSLSTE AQALAGQPGD EIRDLEMVPD |
| 301 | SMLHSSSERP TFIDRLANSL TKRKRSTPQK FVGEKQMRFS LSDLPYDVNS GGYEKDVELV |
| 361 | AHHSLEPGFG SSLAFVGAEH LRPLRLPPTN CISELTPVIS SVYTQMQPLP GRLELPGSRE |
| 421 | AGEGPEDLAD GGPLLYRPRG PLTDPGASPS NGCQDSTDTE SNHEDRVAGV VSLPQGPPPQ |
| 481 | PPPTIVVGRH SPAYAKEDPK PQEGLLRGTP GPSKEVLRVV GESGEPVKAF KCEHCRILFL |
| 541 | DHVMFTIHMG CHGFRDPFEC NICGYHSQDR YEFSSHIVRG EHKVG |

SEQ ID NO: 8 is the human wild type nucleotide sequence corresponding to IKZF4 (nucleotides 1-5506), wherein the underscored bolded “ATG” denotes the beginning of the open reading frame:

| | |
|---|---|
| 1 | gaagctgtcc gtgtcctggg ccccatgacc tctggggcct tggcttcccc agctggcaga |
| 61 | ggattgggcc ttccctaggg cccccccttt ctccctccca cccgcaggcc catccatctc |
| 121 | tctctctctc tcttgcacac actcttgcct ctctcaggca tttgttgtgc agttcctctt |
| 181 | tgtctgctgg gcacgagggg caacagcatc tgcctttccc tccctgtgca cacacccacc |
| 241 | acccaccccc ttcactgtct tggaaaaggg atgctgtagc ctagcatctc ccccactata |
| 301 | tacacatata cattctctcc agccccctcc ccaagcacat ccaagcgtgc tctcccctct |
| 361 | ccttctctcc ctctctctct ctctctctct cacacacaca cacacacaca cactcaacac |
| 421 | acatacaccc tgggctgagc tgctcttgct ggctgcagcc gtgggcctct gctcaccgtg |
| 481 | ccgctgctgc tgcctgcgaa atgacggcgg ttcccctcac ttccaggaat ccacgcttcc |
| 541 | tggaaggtga gtggctgggc tcacccctgc ctgccactga gacgcagaca tgcatacacc |
| 601 | acccgcactc cctcgccgtt tccaaggcgg cggccgcgtt cgcaccccag ggtctcaccg |
| 661 | gcaagggaag gataatctgg agagggatcc ctcaggaggg tgtgttccgg atttcttgcc |
| 721 | tcaggcccaa gactccaacc attttataat ggaatcttta ttttgtgaaa gtagcgggga |
| 781 | ctcatctctg gagaaggagt tcctcggggc cccagtgggg ccctcggtga gcacccccaa |
| 841 | cagccagcac tcttctccta gccgctcact cagtgccaac tccatcaagg tggagatgta |
| 901 | cagcgatgag gagtcaagca gactgctggg gccagatgag cggctcctgg aaaaggacga |
| 961 | cagcgtgatt gtggaagatt cattgtctga gcccctgggc tactgtgatg ggagtgggcc |
| 1021 | agagcctcac tcccctgggg gcatccggct gcccaatggc aagctcaagt gtgacgtctg |
| 1081 | cggcatggtc tgtattggac ccaacgtgct catggtgcac aagcgcagtc acactggtga |
| 1141 | aaggcccttc cattgcaacc agtgtggtgc ctccttcacc cagaagggga acctgctgcg |
| 1201 | ccacatcaag ctgcactctg gggagaagcc ctttaaatgt cccttctgca actatgcctg |
| 1261 | ccgccggcgt gatgcactca ctggtcacct ccgcacacac tcagtctcct ctcccacagt |
| 1321 | gggcaagccc tacaagtgta actactgtgg ccggagctac aaacagcaga gtaccctgga |
| 1381 | ggagcacaag gagcggtgcc ataactacct acagagtctc agcactgaag cccaagcttt |
| 1441 | ggctggccaa ccaggtgacg aaatacgtga cctggagatg gtgccagact ccatgctgca |
| 1501 | ctcatcctct gagcggccaa ctttcatcga tcgtctggcc aatagcctca ccaaacgcaa |
| 1561 | gcgttccaca ccccagaagt ttgtaggcga aaagcagatg cgcttcagcc tctcagacct |
| 1621 | cccctatgat gtgaactcgg gtggctatga aaaggatgtg gagttggtgg cacaccacag |
| 1681 | cctagagcct ggctttggaa gttccctggc ctttgtgggt gcagagcatc tgcgtcccct |
| 1741 | ccgccttcca cccaccaatt gcatctcaga actcacgcct gtcatcagct ctgtctacac |
| 1801 | ccagatgcag cccctccctg gtcgactgga gcttccagga tcccgagaag caggtgaggg |
| 1861 | acctgaggac ctggctgatg gaggtcccct cctctaccgg ccccgaggcc ccctgactga |
| 1921 | ccctggggca tcccccagca atggctgcca ggactccaca gacacagaaa gcaaccacga |
| 1981 | agatcgggtt gcgggggtgg tatccctccc tcagggtccc ccaccccagc cacctcccac |
| 2041 | cattgtggtg ggccggcaca gtcctgccta cgccaaagag gaccccaagc cacaggaggg |
| 2101 | gttattgcgg ggcaccccag gcccctccaa ggaagtgctt cgggtggtgg gcgagagtgg |
| 2161 | tgagcctgtg aaggccttca agtgtgagca ctgccgtatc ctcttcctgg accacgtcat |
| 2221 | gttcactatc cacatgggct gccatggctt cagagaccct tttgagtgca acatctgtgg |
| 2281 | ttatcacagc caggaccggt acgaattctc ttcccacatt gtccgggggg agcataaggt |
| 2341 | gggctagcaa cctctccctc tctcctcagt ccaccactcc actgccctga ctacaggcat |
| 2401 | tgatccctgt ccccaccatt tcccaaggag ttttgctttg tagccctcac tactggccac |
| 2461 | ctgacctcac acctgaccct gacccctcct cacctattct cttcctctat cctgaccgat |
| 2521 | gtaagcattg tgatgaaaca gatcttttgc ttatgttttt cctttttatc ttctctcatc |
| 2581 | ccagcatact gagttattta ttaattagtt gatttatttt tgccttttta aattttaact |
| 2641 | tatatcagtc acttgccact cccccaccct cctgtccaca actcctttcc actttaggcc |
| 2701 | aatttttctc tcttagatct tccagcagcc ccaggggtag gaagctcctc ttagtactaa |
| 2761 | gagacttcaa gcttcttgct ttaagtcctc accctttaca ttatctaatt cttcagtttt |
| 2821 | gatgctgata cctgcccccg gccctacctt agctctgtgg cattatatct cctctctggg |
| 2881 | actcttcaac ctggtactcc atacctcttg tgccctctca ctttaggcag cttgcactat |
| 2941 | tcttgaatga atgaagaatt atttcctcat ttggaagtag gagggactga agaaattctc |
| 3001 | cccaggcact gtgggactga gagtcctatt cccctagtaa taggtcatat tcccctagta |
| 3061 | atatgagttc tcaaagccta cattcaggat ctccctctag gatgtgatag atctggtccc |
| 3121 | tctccttgaa ctacccctcc acacgctcta gtcccttcaa cctaccggtc tattaagtgg |
| 3181 | tggcttttct ctccttggag tgccccaatt ttatattctc aggggccaag gctaggtctg |
| 3241 | caaccctctg tctctgacag attgggagcc acaggtgcct aattgggaac cagggcatgg |
| 3301 | gaaaggagtg ggtcaaaatt cttctctttc tcctccacct ctcaaacttc ttcactatag |
| 3361 | tgaccttcct aggctctcag gggctccttc agtccccatc ctatgagaaa ctagtgggtt |
| 3421 | gctgcctgat gacaaggggt tgtttcagcc cctcagtcat gctgccttct gctgctccct |
| 3481 | cccagcagga ttcaccctct cattcccggg ctcctgggcc ctgttcttag gatcagtggc |
| 3541 | agggagaaac gggtatctct tttctctctt ctaattttca gtataaccaa aaattatccc |
| 3601 | agcatgagca cgggcacgtg cccttcaccc cattccaccc ttgttccagc aagactggga |
| 3661 | tgggtacaac tgaactgggg tcttccttta ctaccccctt ctacactcag ctcccagaca |
| 3721 | cagggtagga ggggggactg ctggctactg cagagaccct tggctatttg agtaacctag |
| 3781 | gattagtgag aaggggcaga aggagataca actccactgc aagtggaggt ttctttctac |
| 3841 | aagagttttc tgcccaaggc cacagccatc ccactctctg cttccttgag attcaaacca |
| 3901 | aaggctgttt ttctatgttt aaagaaaaaa aaaagtaaaa accaaacaca acacctcaca |
| 3961 | agttgtaact cttggtcctt ctctctctcc ttttctcttc ccttccttcc ccttccatct |
| 4021 | ttctttccac atgtcctttc cttattggct cttttacctc ctacttttct cactccctat |
| 4081 | cagggatatt ttgggggggg atggtaaagg gtgggctaag gaacagaccc tgggattagg |
| 4141 | gccttaaggg ctctgagagg agtctacctt gccttcttat gggaagggag accctaaaaa |
| 4201 | actttctcct ctttgtcctc ctttttctcc cccactctga ggtttcccca agagaaccag |
| 4261 | attggcaggg agaagcattg tggggcaatt gttcctcctt gacaatgtag caataaatag |
| 4321 | atgctgccaa gggcagaaaa tggggaggtt agctcagagc agagtagtct ctagagaaag |
| 4381 | gaagaatcct caacggcacc ctggggtgct agctcctttt tagaatgtca gcagagctga |
| 4441 | gattaatatc tgggcttttc ctgaactatt ctggttattg agcccttcct gttagaccta |
| 4501 | ccgcctccca cctcttctgt gtctgctgtg tatttggtga cacttcataa ggactagtcc |
| 4561 | cttctggggt atcagagcct tagggtgccc ccatcccctt ccccagtcaa ctgtggcacc |
| 4621 | tgtaacctcc cggaacatga aggactatgc tctgaggcta tactctgtgc ccatgagagc |
| 4681 | agagactgga agggcaagac caggtgctaa ggaggggaga gggggcatcc tgtctctctc |
| 4741 | cagaccatca ctgcacttta accagggtct taggtacaaa atcctacttt tcagagcctt |
| 4801 | ccagctctgg aacctcaaac atcctcatgc tctctcccag ctccttttgc ataaaaaaaa |
| 4861 | aagtaaagaa aaagaaaaaa aaatacacac acactgaaac ccacatggag aaaagaggtg |
| 4921 | tttcctttta tattgctatt caaaatcaat accaccaaca aaatatttct aagtagacac |
| 4981 | ttttccagac ctttgttttt ttgtgtcagt gtccaagctg cagataggat tttgtaatac |
| 5041 | ttctggcagc ttctttcctt gtgtacataa tatatatata tacatatata tatatatttt |
| 5101 | taatcagaag ttatgaagaa caaaaagaaa aaataaacac agaagcaagt gcaataccac |
| 5161 | ctctcttctc cctctctcct agggtttcct ttgtagccta tgtttggtgt ctcttttgac |
| 5221 | ctttacccct tcacctcctc ctctcttctt ctgattcccc tccccccctt ttttaaagag |
| 5281 | tttttctcct ttctcaaggg gagttaaact agcttttgag acttattgca aagcattttg |
| 5341 | tatatgtaat atattgtaag taaatatttg tgtaacggag atatactact gtaagttttg |
| 5401 | tactgtactg gctgaaagtc tgttataaat aaacatgagt aatttaacac caaaaaaaaa |
| 5461 | aaaaaaaaaa aaaaaaaaaa aaaaaaaaaa aaaaaaaaaa aaaaaa |

The polypeptide sequence of human PTGER4 is depicted in SEQ ID NO: 9. The nucleotide sequence of human PTGER4 is shown in SEQ ID NO: 10. Sequence information related to PTGER4 is accessible in public databases by GenBank Accession numbers NM_{—}000958 (for mRNA) and NP_{—}000949 (for protein).

PTGER4 (prostaglandin E receptor 4) is a member of the G-protein coupled receptor family. It is one of four receptors identified for prostaglandin E2 (PGE2), and can activate T-cell factor signaling (Mum J, Alibert O, Wu N, Tendil S, Gidrol X. J Exp Med. 2008 Dec. 22; 205(13):3091-103).

SEQ ID NO: 9 is the human wild type amino acid sequence corresponding to PTGER4 (residues 1-488):

| | |
|---|---|
| 1 | MSTPGVNSSA SLSPDRLNSP VTIPAVMFIF GVVGNLVAIV VLCKSRKEQK ETTFYTLVCG |
| 61 | LAVTDLLGTL LVSPVTIATY MKGQWPGGQP LCEYSTFILL FFSLSGLSII CAMSVERYLA |
| 121 | INHAYFYSHY VDKRLAGLTL FAVYASNVLF CALPNMGLGS SRLQYPDTWC FIDWTTNVTA |
| 181 | HAAYSYMYAG FSSFLILATV LCNVLVCGAL LRMHRQFMRR TSLGTEQHHA AAAASVASRG |
| 241 | HPAASPALPR LSDFRRRRSF RRIAGAEIQM VILLIATSLV VLICSIPLVV RVFVNQLYQP |
| 301 | SLEREVSKNP DLQAIRIASV NPILDPWIYI LLRKTVLSKA IEKIKCLFCR IGGSRRERSG |
| 361 | QHCSDSQRTS SAMSGHSRSF ISRELKEISS TSQTLLPDLS LPDLSENGLG GRNLLPGVPG |
| 421 | MGLAQEDTTS LRTLRISETS DSSQGQDSES VLLVDEAGGS GRAGPAPKGS SLQVTFPSET |
| 481 | LNLSEKCI |

SEQ ID NO: 10 is the human wild type nucleotide sequence corresponding to PTGER4 (nucleotides 1-3432), wherein the underscored bolded “ATG” denotes the beginning of the open reading frame:

| | |
|---|---|
| 1 | gcgagagcgg agctccaagc ccggcagccc gagaggaaga tgaacagccc caggccagag |
| 61 | cctctggcag agtggacccc gagccgcccc caggtagcca ggagcggcct cagcggcagc |
| 121 | cgcaaactcc agtagccgcc cgtgctgccc gtggctgggg cggagggcag ccagagctgg |
| 181 | ggaccaaggc tccgcgccac ctgcgcgcac agcctcacac ctgaacgctg tcctcccgca |
| 241 | gacgagaccg gcgggcactg caaagctggg actcgtcttt gaaggaaaaa aaatagcgag |
| 301 | taagaaatcc agcaccattc ttcactgacc catcccgctg cacctcttgt ttcccaagtt |
| 361 | tttgaaagct ggcaactctg acctcggtgt ccaaaaatcg acagccactg agaccggctt |
| 421 | tgagaagccg aagatttggc agtttccaga ctgagcagga caaggtgaaa gcaggttgga |
| 481 | ggcgggtcca ggacatctga gggctgaccc tgggggctcg tgaggctgcc accgctgctg |
| 541 | ccgctacaga cccagccttg cactccaagg ctgcgcaccg ccagccacta tcatgtccac |
| 601 | tcccggggtc aattcgtccg cctccttgag ccccgaccgg ctgaacagcc cagtgaccat |
| 661 | cccggcggtg atgttcatct tcggggtggt gggcaacctg gtggccatcg tggtgctgtg |
| 721 | caagtcgcgc aaggagcaga aggagacgac cttctacacg ctggtatgtg ggctggctgt |
| 781 | caccgacctg ttgggcactt tgttggtgag cccggtgacc atcgccacgt acatgaaggg |
| 841 | ccaatggccc gggggccagc cgctgtgcga gtacagcacc ttcattctgc tcttcttcag |
| 901 | cctgtccggc ctcagcatca tctgcgccat gagtgtcgag cgctacctgg ccatcaacca |
| 961 | tgcctatttc tacagccact acgtggacaa gcgattggcg ggcctcacgc tctttgcagt |
| 1021 | ctatgcgtcc aacgtgctct tttgcgcgct gcccaacatg ggtctcggta gctcgcggct |
| 1081 | gcagtaccca gacacctggt gcttcatcga ctggaccacc aacgtgacgg cgcacgccgc |
| 1141 | ctactcctac atgtacgcgg gcttcagctc cttcctcatt ctcgccaccg tcctctgcaa |
| 1201 | cgtgcttgtg tgcggcgcgc tgctccgcat gcaccgccag ttcatgcgcc gcacctcgct |
| 1261 | gggcaccgag cagcaccacg cggccgcggc cgcctcggtt gcctcccggg gccaccccgc |
| 1321 | tgcctcccca gccttgccgc gcctcagcga ctttcggcgc cgccggagct tccgccgcat |
| 1381 | cgcgggcgcc gagatccaga tggtcatctt actcattgcc acctccctgg tggtgctcat |
| 1441 | ctgctccatc ccgctcgtgg tgcgagtatt cgtcaaccag ttatatcagc caagtttgga |
| 1501 | gcgagaagtc agtaaaaatc cagatttgca ggccatccga attgcttctg tgaaccccat |
| 1561 | cctagacccc tggatatata tcctcctgag aaagacagtg ctcagtaaag caatagagaa |
| 1621 | gatcaaatgc ctcttctgcc gcattggcgg gtcccgcagg gagcgctccg gacagcactg |
| 1681 | ctcagacagt caaaggacat cttctgccat gtcaggccac tctcgctcct tcatctcccg |
| 1741 | ggagctgaag gagatcagca gtacatctca gaccctcctg ccagacctct cactgccaga |
| 1801 | cctcagtgaa aatggccttg gaggcaggaa tttgcttcca ggtgtgcctg gcatgggcct |
| 1861 | ggcccaggaa gacaccacct cactgaggac tttgcgaata tcagagacct cagactcttc |
| 1921 | acagggtcag gactcagaga gtgtcttact ggtggatgag gctggtggga gcggcagggc |
| 1981 | tgggcctgcc cctaagggga gctccctgca agtcacattt cccagtgaaa cactgaactt |
| 2041 | atcagaaaaa tgtatataat aggcaaggaa agaaatacag tactgtttct ggacccttat |
| 2101 | aaaatcctgt gcaatagaca catacatgtc acatttagct gtgctcagaa gggctatcat |
| 2161 | catcctacaa ctcacattag agaacatcct ggcttttgag cacttttcaa acaatcaagt |
| 2221 | tgactcacgt gggtcctgag gcctgcagca cgtcggatgc taccccacta tgacagagga |
| 2281 | ttgtggtcac aacttgatgg ctgcgaagac ctaccctccg tttttctact agataggagg |
| 2341 | atggtagaag tttggctgct gtcataacat ccagagcttt gtcgtatttg gcacacagca |
| 2401 | gaggcccaga tattagaaag gctctattcc aataaactat gaggactgcc ttatggatga |
| 2461 | tttaagtgtc tcactaaagc atgaaatgtg aatttttatt gttgtacata cgatttaagg |
| 2521 | tatttaaagt attttcttct ctgtgagaag gtttattgtt aatacaaggt ataataaaat |
| 2581 | tatcgcaacc cctctccttc cagtataacc agctgaagtt gcagatgtta gatatttttc |
| 2641 | ataaacaagt tcgagtcaaa gttgaaaatt catagtaaga ttgatatcta taaaatagat |
| 2701 | ataaattttt aagagaaaga atttagtatt atcaaaggga taaagaaaaa aatactattt |
| 2761 | aagatgtgaa aattacagtc caaaatactg ttctttccag gctatgtata aaatacatag |
| 2821 | tgaaaattgt ttagtgatat tacatttatt tatccagaaa actgtgattt caggagaacc |
| 2881 | taacatgctg gtgaatattt tcaacttttt ccctcactaa ttggtacttt taaaaacata |
| 2941 | acataaattt tttgaagtct ttaataaata acccataatt gaagtgtata atataaaaaa |
| 3001 | ttttaaaaat ctaagcagct tattgtttct ctgaaagtgt gtgtagtttt actttcctaa |
| 3061 | ggaattacca agaatatcct ttaaaattta aaaggatggc aagttgcatc agaaagcttt |
| 3121 | attttgagat gtaaaaagat tcccaaacgt ggttacatta gccattcatg tatgtcagaa |
| 3181 | gtgcagaatt ggggcactta atggtcacct tgtaacagtt ttgtgtaact cccagtgatg |
| 3241 | ctgtacacat atttgaaggg tctttctcaa agaaatatta agcatgtttt gttgctcagt |
| 3301 | gtttttgtga attgcttggt tgtaattaaa ttctgagcct gatattgata tggttttaag |
| 3361 | aagcagttgt accaagtgaa attattttgg agattataat aaatatatac attcaaaaaa |
| 3421 | aaaaaaaaaa aa |

The polypeptide sequence of human PRDX5 is depicted in SEQ ID NO: 11. The nucleotide sequence of human PRDX5 is shown in SEQ ID NO: 12. Sequence information related to PRDX5 is accessible in public databases by GenBank Accession numbers NM_{—}012094 (for mRNA) and NP_{—}036226 (for protein).

PRDX5 (peroxiredoxin-5) is a member of the peroxiredoxin family of antioxidant enzymes. It has been reported to play an antioxidant protective role in different tissues under normal conditions and during inflammatory processes. This protein interacts with peroxisome receptor 1 (Nguyên-Nhu N T, et al., Biochim Biophys Acta. 2007 July-August; 1769(7-8):472-83).

SEQ ID NO: 11 is the human wild type amino acid sequence corresponding to PRDX5 (residues 1-214):

| | |
|---|---|
| 1 | MGLAGVCALR RSAGYILVGG AGGQSAAAAA RRCSEGEWAS GGVRSFSRAA AAMAPIKVGD |
| 61 | AIPAVEVFEG EPGNKVNLAE LFKGKKGVLF GVPGAFTPGC SKTHLPGFVE QAEALKAKGV |
| 121 | QVVACLSVND AFVTGEWGRA HKAEGKVRLL ADPTGAFGKE TDLLLDDSLV SIFGNRRLKR |
| 181 | FSMVVQDGIV KALNVEPDGT GLTCSLAPNI ISQL |

SEQ ID NO: 12 is the human wild type nucleotide sequence corresponding to PRDX5 (nucleotides 1-959), wherein the underscored bolded “ATG” denotes the beginning of the open reading frame:

| | |
|---|---|
| 1 | gcagtggagg cggcccaggc ccgccttccg cagggtgtcg ccgctgtgcc gctagcggtg |
| 61 | ccccgcctgc tgcggtggca ccagccagga ggcggagtgg aagtggccgt ggggcgggta |
| 121 | tgggactagc tggcgtgtgc gccctgagac gctcagcggg ctatatactc gtcggtgggg |
| 181 | ccggcggtca gtctgcggca gcggcagcaa gacggtgcag tgaaggagag tgggcgtctg |
| 241 | gcggggtccg cagtttcagc agagccgctg cagccatggc cccaatcaag gtgggagatg |
| 301 | ccatcccagc agtggaggtg tttgaagggg agccagggaa caaggtgaac ctggcagagc |
| 361 | tgttcaaggg caagaagggt gtgctgtttg gagttcctgg ggccttcacc cctggatgtt |
| 421 | ccaagacaca cctgccaggg tttgtggagc aggctgaggc tctgaaggcc aagggagtcc |
| 481 | aggtggtggc ctgtctgagt gttaatgatg cctttgtgac tggcgagtgg ggccgagccc |
| 541 | acaaggcgga aggcaaggtt cggctcctgg ctgatcccac tggggccttt gggaaggaga |
| 601 | cagacttatt actagatgat tcgctggtgt ccatctttgg gaatcgacgt ctcaagaggt |
| 661 | tctccatggt ggtacaggat ggcatagtga aggccctgaa tgtggaacca gatggcacag |
| 721 | gcctcacctg cagcctggca cccaatatca tctcacagct ctgaggccct gggccagatt |
| 781 | acttcctcca cccctcccta tctcacctgc ccagccctgt gctggggccc tgcaattgga |
| 841 | atgttggcca gatttctgca ataaacactt gtggtttgcg gccaaaaaaa aaaaaaaaaa |
| 901 | aaaaaaaaaa aaaaaaaaaa aaaaaaaaaa aaaaaaaaaa aaaaaaaaaa aaaaaaaaa |

The polypeptide sequence of human STX17 is depicted in SEQ ID NO: 13. The nucleotide sequence of human STX17 is shown in SEQ ID NO: 14. Sequence information related to STX17 is accessible in public databases by GenBank Accession numbers NM_{—}017919 (for mRNA) and NP_{—}060389 (for protein).

Syntaxin-17 (STX17) is a member of the syntaxin family and recently was reported to be a Ras-interacting protein (Südhof TC, Rothman J E. Science. 2009 Jan. 23; 323(5913):474-7; Zhang et al., J Histochem Cytochem. 2005 November; 53(11):1371-82; and Steegmaier, M., et al., J. Biol. Chem. 273 (51), 34171-34179 (1998)).

SEQ ID NO: 13 is the human wild type amino acid sequence corresponding to STX17 (residues 1-302):

| | |
|---|---|
| 1 | MSEDEEKVKL RRLEPAIQKF IKIVIPTDLE RLRKHQINIE KYQRCRIWDK LHEEHINAGR |
| 61 | TVQQLRSNIR EIEKLCLKVR KDDLVLLKRM IDPVKEEASA ATAEFLQLHL ESVEELKKQF |
| 121 | NDEETLLQPP LTRSMTVGGA FHTTEAEASS QSLTQIYALP EIPQDQNAAE SWETLEADLI |
| 181 | ELSQLVTDFS LLVNSQQEKI DSIADHVNSA AVNVEEGTKN LGKAAKYKLA ALPVAGALIG |
| 241 | GMVGGPIGLL AGFKVAGIAA ALGGGVLGFT GGKLIQRKKQ KMMEKLTSSC PDLPSQTDKK |
| 301 | CS |

SEQ ID NO: 14 is the human wild type nucleotide sequence corresponding to STX17 (nucleotides 1-6910), wherein the underscored bolded “ATG” denotes the beginning of the open reading frame:

| | |
|---|---|
| 1 | ctcgtgatgc cccgccccgt cgctcctgcg cctgcgccgt gcccaccgac cggcctcgag |
| 61 | cgccccggcg ggaggttttt ctatatgagt ggagaagaca gctgttacca gggaggtcat |
| 121 | acaacatttt tttaggatgt ctgaagatga agaaaaagtg aaattacgcc gtcttgaacc |
| 181 | agctatccag aaattcatta agatagtaat cccaacagac ctggaaaggt taagaaagca |
| 241 | ccagataaat attgagaagt atcaaaggtg cagaatctgg gacaagttgc atgaagagca |
| 301 | tatcaatgca ggacgtacag ttcagcaact ccgatccaat atccgagaaa ttgagaaact |
| 361 | ttgtttgaaa gtccgaaagg atgacctagt acttctgaag agaatgatag atcctgttaa |
| 421 | agaagaagca tcagcagcaa cagcagaatt tctccaactc catttggaat ctgtagaaga |
| 481 | acttaagaag caatttaatg atgaagaaac tttgctacag cctcctttga ccagatccat |
| 541 | gactgttggt ggagcatttc atactactga agctgaagct agttctcaga gtttgactca |
| 601 | gatatatgcc ttacctgaaa ttcctcaaga tcaaaatgct gcagaatcgt gggaaacctt |
| 661 | agaagcggac ttaattgaac ttagccaact ggtcactgac ttctctctcc tagtgaattc |
| 721 | tcagcaggag aagattgaca gcattgcaga ccatgtcaac agtgctgctg tgaatgttga |
| 781 | agagggaacc aaaaacttag ggaaggctgc aaaatacaag ctggcagctc tgcctgtggc |
| 841 | aggtgcactc atcgggggaa tggtaggggg tcctattggc ctccttgcag gcttcaaagt |
| 901 | ggcaggaatt gcagctgcac ttggtggtgg ggtgttgggc ttcacaggtg gaaaattgat |
| 961 | acaaagaaag aaacagaaaa tgatggagaa gctcacttcc agctgtccag atcttcccag |
| 1021 | ccaaactgac aagaaatgca gttaaaaacc aaatttcagt attattggtg ccaacatgtc |
| 1081 | tatcctgagg acctttgctg ctgttggaca ctccgtcacc ttttggaaca caagtatatc |
| 1141 | aagatagtgg ctactgatgt tcaagtggga ttgaagtgtg ataaatggat atattttgtt |
| 1201 | gtttgctggg gtgttcatgg agatgttaag agattgaggc cctgggctga gggtatataa |
| 1261 | tgtatgtcag gtaaagtttg aagactgcca aggagcagat tttctccctg gaaatgtgaa |
| 1321 | aactgaacct ataactctga taaggacttg agatgtgtag aaacgttggg ttatggaaga |
| 1381 | ctagtttctt ccataaccct gaattggaga ccttaatgct aagtgtagat tattgaggtt |
| 1441 | tgttagtgag gaaaagaata agagttcaga agcctttgtt atcagatagc gaaatcaggg |
| 1501 | cctagtgagg agcacaggtc gactacataa tggagtccat tggcgaaccc tattgcaatt |
| 1561 | tggtccaact atatcttctg gtgaaggaaa ttaatgatgt aagaaaatgc aagaggctca |
| 1621 | acttctcttc caaaaatctt ctggcttctg aactcttcct ctgcctctct ttaaataaat |
| 1681 | aacacagaat ttcaagtggt aggagactta ttaagccagt caccaagctt ggtctgtcag |
| 1741 | cctgtcttct aacacctcaa agatcttgtg ccctgtgctg tccctccctt gtaattatga |
| 1801 | aaagttcttt ggtttctggg gtgaattcta cccatgtata atgaggaatt ctctcataac |
| 1861 | cttttttgtc ttgtctgtca tctctgttca tcccttccta taacctctag gtaaaaagaa |
| 1921 | aagaaaaaaa gaaatttcga gatattttca acattgttag agtttgggct aaaatgagca |
| 1981 | aggagaaaaa aaccaccaag aacatttcct ggggcatgtt ccagttttga ggggtgatat |
| 2041 | atctgccaga tagggggtat ctgacccagt cttcttttca gctggtctct ggggggagct |
| 2101 | gagaactcgc ttgctacctc acatcctttt ccccagactt tttatctcct atgcatccct |
| 2161 | ttgctttcta tagctggtgt ttcttcccca aaatggcgtt cccatgctta cctttctcac |
| 2221 | attctagaca atgatggaca aagacgcatg caagactcag acccggggaa tggtgtggtg |
| 2281 | ctaatctcaa cacctgacat tcacagcaag catggcccag cccaactgca tgtctatctc |
| 2341 | aaaccgcaga aaggctttaa tactggaaaa aaagaattca agactacagg cagctcccct |
| 2401 | ctgtacccca actcatttaa aataggagga atcacttttt gccttactta acgctttttt |
| 2461 | ctgagcacag ggatgggcac ctgcacccca gaaggtgtga gctgtctctc tgccaggagc |
| 2521 | taaggttcat taggggattg gatggtttat cacttctttc tttctgagtt tacttttagt |
| 2581 | aacttttatt gatggctacc tttcatgtcc ctgtctaaag agactttctc tttcatacgt |
| 2641 | cttaaatctc atcaatgaaa tccagtgaaa cagcaccatt tcttagtatc attaaataac |
| 2701 | tagaaagtat caagtattgc tctctgctgc tttatatcat taacatatta ataataccaa |
| 2761 | gaaggaaata ctttgaataa gtgtcagatt ctgatccagt attggacacc tgtgatattg |
| 2821 | gacacctgtg aggctgggat aattactttt gaattacacc tcttctctag tttctggacc |
| 2881 | ttgctctgtc actttaacac agggtgatca aacctgaatg aggatcagaa ctcacccagg |
| 2941 | cacatactaa agcaagattc ctaaacctca gttccagggg taattctgac atcacccgtc |
| 3001 | cagcatagtc agctgaaatt ataaatctaa gaaacagtta catcaagatt ctgctgtgtc |
| 3061 | atttaattct gaaactccca gtattctacc cttcttcatc actgcatatt accccactct |
| 3121 | tccatcccaa attggctatc ctttcagccc accaacttag cggcagcact agggattcat |
| 3181 | tataaggtaa atctggttta cataaagacc tgaaggaggc ctgtatttga agctcacact |
| 3241 | tggtattggt atctctcatt tttactgagc cagtgtggaa taccactgta tgtactcata |
| 3301 | taagcccttg acttttactg ctcatcagga ttggaatatt actctagcag tcttcacaca |
| 3361 | taggcaagtt acagtccttt taaaaagtat ctcatttccc tataatggaa cctaatagcc |
| 3421 | aactttttca tagaaattgc tagaagagtt tgatcaacta taaatgataa agtgtttata |
| 3481 | agcatagtca gtgtgacaca gaaaccaatc ttaaaattga atttaatgtt ttatcatatc |
| 3541 | agattaaata ttttctccat gtcttatttt tactgcaaca agttagaaag tgggaacact |
| 3601 | ttgattaatg tcttaaaatt tgtgggccct catttggata aaggcagcaa tcctaaggac |
| 3661 | tttttttttt tttttaacat aatctgagaa tttctctgta gagcagagac tttcaaacct |
| 3721 | tttggctgta acccacagta aaaaacgcat ttatatcaaa ccttagaata tgtttaatga |
| 3781 | acaatactta ccattctgat gctttttatt gtttcagttt ttaaaatatg ccagttgcaa |
| 3841 | cccactaaat tgatatctac caatgggttg caacccttag cttgaaaaaa acaccctcac |
| 3901 | agaggaactg gtatttcttg aataccttct gtttgccagg cacttcacca ggcattttac |
| 3961 | aagtaaggaa actgggcttc agagaaaata atttgcagag gtttactcaa ctacaaaggg |
| 4021 | gtgaagccag gaatgttaac taggtctgtt gagctacaaa aacttttatg tctctcagac |
| 4081 | tatacagcct ctatacaaaa ttgagatggg ggttgggggc aggggctcat gcctgcaatc |
| 4141 | ccagcactta gggaggcaga ggccagagga tcacttgagc ccaggagttt gagaccagcc |
| 4201 | tgggcaacat agtgagactc ttgtctgtat gaaaaaaatt aagaattagc tgggtgtggc |
| 4261 | atagcacaca cctgtggtcc cagctacatg ggaagctgaa gtgggaggat cacttgaact |
| 4321 | caggagcagc cttggtgaca gaacaagacc ctctctcaaa aaaatattta aaaaaaggtg |
| 4381 | ggtcatccat tctcctttac caaacaggct ttgaaatgac acattccatt catttgcatc |
| 4441 | tttttaaaaa acttctgatt ccttactgag tgtccagcag cctcaaagtt tttaatggta |
| 4501 | gctgatgcag acataaacag tgctcaattt ggcccttaaa ctataaaatc aagaaagagt |
| 4561 | atttcaatcc catccacctg cctgcaagat ttcttaatgt tcactagtta taaccattgt |
| 4621 | ttaaacagtg ctttttgtgt aatttaaaaa taaactttaa tgctttttaa aacaaattta |
| 4681 | tcataattca tagatcaaat gattatcctt taaaatgata cccttgggaa atcatgtact |
| 4741 | tactgtagtg atgctagtat taatattact tagaccaatt ttgaaactgt tctttcagaa |
| 4801 | ttgcctccaa agacattttg cagatcatcc cagaaaaggg ggtatgatgg tgctgtgtag |
| 4861 | aactgaccag agttcctgga ggattttgag gttatactga aactgagtgc tgtacaggga |
| 4921 | gaattgcatg agtccagaaa cttccttctg tgggctgcct gccttcctgc cctcccttaa |
| 4981 | gtgctctaag atttttgtac aggagtaaga atcaaatact ggtaacatca atcacaagaa |
| 5041 | gttgaggaaa cctgtaatat agctagataa tatacaacgt ttgtcttcca tcagagtgca |
| 5101 | gaaaccaaac catgctttgt gttaacctta aatatgaaag gtgtttctca gggtcccctt |
| 5161 | tgtccttcgt tgctgccata tgaaatctta caaggaagga tgaggaaaag cctgggggga |
| 5221 | ggttctcctc ggaaatgagg tggttttttt tgttattaag tagaacgtgg ctgtggttca |
| 5281 | caggtactta acgaatgtta gatgatgttc ttaagtaatc agaggcctaa taaaaggcag |
| 5341 | gggagtttct cttctagcct aaattaatat taaaagttca ggggtatttt ttgtttttaa |
| 5401 | attaatactt tattgttttt aacaggtggt tctcataatt tacattcatt aatttgatgc |
| 5461 | ccttttacaa agaaacttct taggtattat aaaccatcaa tgtaaaggat ccacatggta |
| 5521 | tgtatccaca ttgctactct caaatagaaa tgggagataa gaaatatatc tgtgcaatat |
| 5581 | taaattgaaa aaaaaaaacc cataaaaagt gtcaaaggca aataatttgc tctagatcac |
| 5641 | aaaactagtt agcacaaggc taggattata accagggtct aggaaaaaat cctgaaggtg |
| 5701 | atttaactga gtgttaggcc ctgtcaagcc acctgctaag gctcatggtc tttcagacta |
| 5761 | gcttcaacat tccaaatcag gcaatagcta caacggaaag ataattggac ggggaatcct |
| 5821 | gagatcagag tcctagtttg gctttgtctc ttgtagcagg attttttaaa tcaggggcag |
| 5881 | ctctcttctc ccatcccagc catgaatctt tcaaccttag tggtcaccaa cttgactcca |
| 5941 | ttccttatat caagacttgt cctgtcaatt ctcccttaaa tgttagttgc atccatttct |
| 6001 | aaatatatcc atggccatca ccctagtaaa aagactatta cctcacaccc cgcacttgat |
| 6061 | cttcccccaa ctttaagtga ctcagttcct tatatcactg ccacaagaat taacaaccat |
| 6121 | gtccatcttt catttttctg ctgaaagatt ttcagtggtt cccactgaat accaaataaa |
| 6181 | gttcgaatcc cttagattgg cattcacagc cttctacgtt ctggccccag ctttatctct |
| 6241 | tgaaactcac tactcaccat ctgacaatgc cactaaaaat ccacaagagt gattttaagg |
| 6301 | tttttctatg gtgaaggttc aaactggtaa taaaccatgt ttacattttt ctggtctaaa |
| 6361 | ataatttcta tattacttta taatagtcag ctgggggtta tttaagctct tggacgagcc |
| 6421 | taaaacttgt atcctgaaga aaatattttt ttccaccaga agaaattgct ttcaatttct |
| 6481 | taaccttcaa aacaatgtca gtgttgtcac ctgtgcattt gatagccaca gcacaagtat |
| 6541 | tcttcaggag cataaatcct ccagccttga atggaccatt gtccagctcc tgtgaaaaac |
| 6601 | ttaatatttg agaaagacat tcaatggtac atgttttctg tacacttcat gagtagttga |
| 6661 | gattttcttg tattaaggtt aatcactaaa aaggtgttta cttgggtttc gttaactaaa |
| 6721 | ccccctaaag atgttttcca ttttattgtt aaacacttgg tgttagcaag ggtcagcacg |
| 6781 | agaaaaggcc caatggcaag aatttctgca aactctgtaa agcttactga attcatttgt |
| 6841 | catttattac attgctgagt ggtgcttgaa taaggaaaca tgcaataaat ttacttattt |
| 6901 | aaccaacaaa |

The polypeptide sequence of human NKG2D is depicted in SEQ ID NO: 15. The nucleotide sequence of human NKG2D is shown in SEQ ID NO: 16. Sequence information related to NKG2D is accessible in public databases by GenBank Accession numbers NM_{—}007360 (for mRNA) and NP_{—}031386 (for protein).

NKG2-D type II integral membrane protein (NKG2D) is a protein encoded by the KLRK1 (killer cell lectin-like receptor subfamily K, member 1) gene. KLRK1 has also been designated as CD314. (Nausch N, Cerwenka A. Oncogene. 2008 Oct. 6; 27(45):5944-58; and González S, et al., Trends Immunol. 2008 August; 29(8):397-403).

SEQ ID NO: 15 is the human wild type amino acid sequence corresponding to NKG2D (residues 1-216):

| | |
|---|---|
| 1 | MGWIRGRRSR HSWEMSEFHN YNLDLKKSDF STRWQKQRCP VVKSKCRENA SPFFFCCFIA |
| 61 | VAMGIRFIIM VTIWSAVFLN SLFNQEVQIP LTESYCGPCP KNWICYKNNC YQFFDESKNW |
| 121 | YESQASCMSQ NASLLKVYSK EDQDLLKLVK SYHWMGLVHI PTNGSWQWED GSILSPNLLT |
| 181 | IIEMQKGDCA LYASSFKGYI ENCSTPNTYI CMQRTV |

SEQ ID NO: 16 is the human wild type nucleotide sequence corresponding to NKG2D (nucleotides 1-1593), wherein the underscored bolded “ATG” denotes the beginning of the open reading frame:

| | |
|---|---|
| 1 | actttcaatt ctagatcagg aactgaggac atatctaaat tttctagttt tatagaaggc |
| 61 | ttttatccac aagaatcaag atcttccctc tctgagcagg aatcctttgt gcattgaaga |
| 121 | ctttagattc ctctctgcgg tagacgtgca cttataagta tttgatgggg tggattcgtg |
| 181 | gtcggaggtc tcgacacagc tgggagatga gtgaatttca taattataac ttggatctga |
| 241 | agaagagtga tttttcaaca cgatggcaaa agcaaagatg tccagtagtc aaaagcaaat |
| 301 | gtagagaaaa tgcatctcca ttttttttct gctgcttcat cgctgtagcc atgggaatcc |
| 361 | gtttcattat tatggtaaca atatggagtg ctgtattcct aaactcatta ttcaaccaag |
| 421 | aagttcaaat tcccttgacc gaaagttact gtggcccatg tcctaaaaac tggatatgtt |
| 481 | acaaaaataa ctgctaccaa ttttttgatg agagtaaaaa ctggtatgag agccaggctt |
| 541 | cttgtatgtc tcaaaatgcc agccttctga aagtatacag caaagaggac caggatttac |
| 601 | ttaaactggt gaagtcatat cattggatgg gactagtaca cattccaaca aatggatctt |
| 661 | ggcagtggga agatggctcc attctctcac ccaacctact aacaataatt gaaatgcaga |
| 721 | agggagactg tgcactctat gcctcgagct ttaaaggcta tatagaaaac tgttcaactc |
| 781 | caaatacgta catctgcatg caaaggactg tgtaaagatg atcaaccatc tcaataaaag |
| 841 | ccaggaacag agaagagatt acaccagcgg taacactgcc aactgagact aaaggaaaca |
| 901 | aacaaaaaca ggacaaaatg accaaagact gtcagatttc ttagactcca caggaccaaa |
| 961 | ccatagaaca atttcactgc aaacatgcat gattctccaa gacaaaagaa gagagatcct |
| 1021 | aaaggcaatt cagatatccc caaggctgcc tctcccacca caagcccaga gtggatgggc |
| 1081 | tgggggaggg gtgctgtttt aatttctaaa ggtaggacca acacccaggg gatcagtgaa |
| 1141 | ggaagagaag gccagcagat cactgagagt gcaaccccac cctccacagg aaattgcctc |
| 1201 | atgggcaggg ccacagcaga gagacacagc atgggcagtg ccttccctgc ctgtgggggt |
| 1261 | catgctgcca cttttaatgg gtcctccacc caacggggtc agggaggtgg tgctgcccca |
| 1321 | gtgggccatg attatcttaa aggcattatt ctccagcctt aagtaagatc ttaggacgtt |
| 1381 | tcctttgcta tgatttgtac ttgcttgagt cccatgactg tttctcttcc tctctttctt |
| 1441 | ccttttggaa tagtaatatc catcctatgt ttgtcccact attgtatttt ggaagcacat |
| 1501 | aacttgtttg gtttcacagg ttcacagtta agaaggaatt ttgcctctga ataaatagaa |
| 1561 | tcttgagtct catgcaaaaa aaaaaaaaaa aaa |

The polypeptide sequence of human ULBP6 is depicted in SEQ ID NO: 17. The nucleotide sequence of human ULBP6 is shown in SEQ ID NO: 18. Sequence information related to ULBP6 is accessible in public databases by GenBank Accession numbers NM_{—}130900 (for mRNA) and NP_{—}570970 (for protein).

ULBP6 is also referred to as RAET1L. It is a ligand that activates the immunoreceptor NKG2D and is involved in NK cell activation (Eagle et al., Eur J. Immunol. 2009 Aug. 5).

SEQ ID NO: 17 is the human wild type amino acid sequence corresponding to ULBP6 (residues 1-246):

| | |
|---|---|
| 1 | MAAAAIPALL LCLPLLFLLF GWSRARRDDP HSLCYDITVI PKFRPGPRWC AVQGQVDEKT |
| 61 | FLHYDCGNKT VTPVSPLGKK LNVTMAWKAQ NPVLREVVDI LTEQLLDIQL ENYTPKEPLT |
| 121 | LQARMSCEQK AEGHSSGSWQ FSIDGQTFLL FDSEKRMWTT VHPGARKMKE KWENDKDVAM |
| 181 | SFHYISMGDC IGWLEDFLMG MDSTLEPSAG APLAMSSGTT QLRATATTLI LCCLLIILPC |
| 241 | FILPGI |

SEQ ID NO: 18 is the human wild type nucleotide sequence corresponding to ULBP6 (nucleotides 1-802), wherein the underscored bolded “ATG” denotes the beginning of the open reading frame:

| | |
|---|---|
| 1 | gatttcatct tccaggatcc accttgatta aatctcttgt ccccagccct cctggtcccc |
| 61 | aatggcagca gccgccatcc cagctttgct tctgtgcctc ccgcttctgt tcctgctgtt |
| 121 | cggctggtcc cgggctaggc gagacgaccc tcactctctt tgctatgaca tcaccgtcat |
| 181 | ccctaagttc agacctggac cacggtggtg tgcggttcaa ggccaggtgg atgaaaagac |
| 241 | ttttcttcac tatgactgtg gcaacaagac agtcacaccc gtcagtcccc tggggaagaa |
| 301 | actaaatgtc acaatggcct ggaaagcaca gaacccagta ctgagagagg tggtggacat |
| 361 | acttacagag caactgcttg acattcagct ggagaattac acacccaagg aacccctcac |
| 421 | cctgcaggca aggatgtctt gtgagcagaa agctgaagga cacagcagtg gatcttggca |
| 481 | gttcagtatc gatggacaga ccttcctact ctttgactca gagaagagaa tgtggacaac |
| 541 | ggttcatcct ggagccagaa agatgaaaga aaagtgggag aatgacaagg atgtggccat |
| 601 | gtccttccat tacatctcaa tgggagactg cataggatgg cttgaggact tcttgatggg |
| 661 | catggacagc accctggagc caagtgcagg agcaccactc gccatgtcct caggcacaac |
| 721 | ccaactcagg gccacagcca ccaccctcat cctttgctgc ctcctcatca tcctcccctg |
| 781 | cttcatcctc cctggcatct ga |

The polypeptide sequence of human ULBP3 is depicted in SEQ ID NO: 19. The nucleotide sequence of human ULBP3 is shown in SEQ ID NO: 20. Sequence information related to ULBP3 is accessible in public databases by GenBank Accession numbers NM_{—}024518 (for mRNA) and NP_{—}078794 (for protein).

ULBP3 (UL16 binding protein 3) is a ligand that activates the immunoreceptor NKG2D and is involved in NK cell activation (Sun, P. D., Immunol Res. 2003; 27(2-3):539-48).

SEQ ID NO: 19 is the human wild type amino acid sequence corresponding to ULBP3 (residues 1-244):

| | |
|---|---|
| 1 | MAAAASPAIL PRLAILPYLL FDWSGTGRAD AHSLWYNFTI IHLPRHGQQW CEVQSQVDQK |
| 61 | NFLSYDCGSD KVLSMGHLEE QLYATDAWGK QLEMLREVGQ RLRLELADTE LEDFTPSGPL |
| 121 | TLQVRMSCEC EADGYIRGSW QFSFDGRKFL LFDSNNRKWT VVHAGARRMK EKWEKDSGLT |
| 181 | TFFKMVSMRD CKSWLRDFLM HRKKRLEPTA PPTMAPGLAQ PKAIATTLSP WSFLIILCFI |
| 241 | LPGI |

SEQ ID NO: 20 is the human wild type nucleotide sequence corresponding to ULBP3 (nucleotides 1-735), wherein the underscored bolded “ATG” denotes the beginning of the open reading frame:

| | |
|---|---|
| 1 | atggcagcgg ccgccagccc cgcgatcctt ccgcgcctcg cgattcttcc gtacctgcta |
| 61 | ttcgactggt ccgggacggg gcgggccgac gctcactctc tctggtataa cttcaccatc |
| 121 | attcatttgc ccagacatgg gcaacagtgg tgtgaggtcc agagccaggt ggatcagaag |
| 181 | aattttctct cctatgactg tggcagtgac aaggtcttat ctatgggtca cctagaagag |
| 241 | cagctgtatg ccacagatgc ctggggaaaa caactggaaa tgctgagaga ggtggggcag |
| 301 | aggctcagac tggaactggc tgacactgag ctggaggatt tcacacccag tggacccctc |
| 361 | acgctgcagg tcaggatgtc ttgtgagtgt gaagccgatg gatacatccg tggatcttgg |
| 421 | cagttcagct tcgatggacg gaagttcctc ctctttgact caaacaacag aaagtggaca |
| 481 | gtggttcacg ctggagccag gcggatgaaa gagaagtggg agaaggatag cggactgacc |
| 541 | accttcttca agatggtctc aatgagagac tgcaagagct ggcttaggga cttcctgatg |
| 601 | cacaggaaga agaggctgga acccacagca ccacccacca tggccccagg cttagctcaa |
| 661 | cccaaagcca tagccaccac cctcagtccc tggagcttcc tcatcatcct ctgcttcatc |
| 721 | ctccctggca tctga |

The polypeptide sequence of human IL-21 is depicted in SEQ ID NO: 21. The nucleotide sequence of human IL-21 is shown in SEQ ID NO: 22. Sequence information related to IL-21 is accessible in public databases by GenBank Accession numbers NM_{—}021803 (for mRNA) and NP_{—}068575 (for protein).

Interleukin 21 is a cytokine that regulates cells of the immune system, including natural killer (NK) cells and cytotoxic T cells. This cytokine induces cell division/proliferation in its target cells. (See Rochman Y, Spolski R, Leonard W J. Nat Rev Immunol. 2009 July; 9(7):480-90; Monteleone, G. et al., Cytokine Growth Factor Rev. 2009 April; 20(2):185-91; and Overwijk W W, Schluns K S. Clin Immunol. 2009 August; 132(2):153-65).

SEQ ID NO: 21 is the human wild type amino acid sequence corresponding to IL-21 (residues 1-162):

| | |
|---|---|
| 1 | MRSSPGNMER IVICLMVIFL GTLVHKSSSQ GQDRHMIRMR QLIDIVDQLK NYVNDLVPEF |
| 61 | LPAPEDVETN CEWSAFSCFQ KAQLKSANTG NNERIINVSI KKLKRKPPST NAGRRQKHRL |
| 121 | TCPSCDSYEK KPPKEFLERF KSLLQKMIHQ HLSSRTHGSE DS |

SEQ ID NO: 22 is the human wild type nucleotide sequence corresponding to IL-IL-21 (nucleotides 1-616), wherein the underscored bolded “ATG” denotes the beginning of the open reading frame:

| | |
|---|---|
| 1 | ctgaagtgaa aacgagacca aggtctagct ctactgttgg tacttatgag atccagtcct |
| 61 | ggcaacatgg agaggattgt catctgtctg atggtcatct tcttggggac actggtccac |
| 121 | aaatcaagct cccaaggtca agatcgccac atgattagaa tgcgtcaact tatagatatt |
| 181 | gttgatcagc tgaaaaatta tgtgaatgac ttggtccctg aatttctgcc agctccagaa |
| 241 | gatgtagaga caaactgtga gtggtcagct ttttcctgct ttcagaaggc ccaactaaag |
| 301 | tcagcaaata caggaaacaa tgaaaggata atcaatgtat caattaaaaa gctgaagagg |
| 361 | aaaccacctt ccacaaatgc agggagaaga cagaaacaca gactaacatg cccttcatgt |
| 421 | gattcttatg agaaaaaacc acccaaagaa ttcctagaaa gattcaaatc acttctccaa |
| 481 | aagatgattc atcagcatct gtcctctaga acacacggaa gtgaagattc ctgaggatct |
| 541 | aacttgcagt tggacactat gttacatact ctaatatagt agtgaaagtc atttctttgt |
| 601 | attccaagtg gaggag |

The polypeptide sequence of a human HLA Class II Region protein, such as HLA-DQA2 is depicted in SEQ ID NO: 23. The nucleotide sequence of a human HLA Class II Region protein, such as HLA-DQA2 is shown in SEQ ID NO: 24. Sequence information related to HLA Class II Region proteins, such as HLA-DQA2 is accessible in public databases by GenBank Accession numbers NM_{—}020056 (for mRNA) and NP_{—}064440 (for protein).

SEQ ID NO: 23 is the human wild type amino acid sequence corresponding to HLA-DQA2 (residues 1-255):

| | |
|---|---|
| 1 | MILNKALLLG ALALTAVMSP CGGEDIVADH VASYGVNFYQ SHGPSGQYTH EFDGDEEFYV |
| 61 | DLETKETVWQ LPMFSKFISF DPQSALRNMA VGKHTLEFMM RQSNSTAATN EVPEVTVFSK |
| 121 | FPVTLGQPNT LICLVDNIFP PVVNITWLSN GHSVTEGVSE TSFLSKSDHS FFKISYLTFL |
| 181 | PSADEIYDCK VEHWGLDEPL LKHWEPEIPA PMSELTETLV CALGLSVGLM GIVVGTVFII |
| 241 | QGLRSVGASR HQGLL |

SEQ ID NO: 24 is the human wild type nucleotide sequence corresponding to HLA-DQA2 (nucleotides 1-1709), wherein the underscored bolded “ATG” denotes the beginning of the open reading frame:

| | |
|---|---|
| 1 | tcctcacaat tgctctacag ctcagagcag caactgctga ggctgccttg ggaagaagat |
| 61 | gatcctaaac aaagctctgc tgctgggggc cctcgccctg actgccgtga tgagcccctg |
| 121 | tggaggtgaa gacattgtgg ctgaccatgt tgcctcctat ggtgtgaact tctaccagtc |
| 181 | tcacggtccc tctggccagt acacccatga atttgatgga gacgaggagt tctatgtgga |
| 241 | cctggagacg aaagagactg tctggcagtt gcctatgttt agcaaattta taagttttga |
| 301 | cccgcagagt gcactgagaa atatggctgt gggaaaacac accttggaat tcatgatgag |
| 361 | acagtccaac tctaccgctg ccaccaatga ggttcctgag gtcacagtgt tttccaagtt |
| 421 | tcctgtgacg ctgggtcagc ccaacaccct catctgtctt gtggacaaca tctttcctcc |
| 481 | tgtggtcaac atcacctggc tgagcaatgg gcactcagtc acagaaggtg tttctgagac |
| 541 | cagcttcctc tccaagagtg atcattcctt cttcaagatc agttacctca ccttcctccc |
| 601 | ttctgctgat gagatttatg actgcaaggt ggagcactgg ggcctggacg agcctcttct |
| 661 | gaaacactgg gagcctgaga ttccagcccc tatgtcagag ctcacagaga ctttggtctg |
| 721 | cgccctgggg ttgtctgtgg gcctcatggg cattgtggtg ggcactgtct tcatcatcca |
| 781 | aggcctgcgt tcagttggtg cttccagaca ccaagggctc ttatgaatcc catcctgaaa |
| 841 | aggaaggtgc atcaccatct acaggagaag aagaatggac ttgctaaatg acctagcact |
| 901 | attctctggc ctgatttatc atatcccttt tctcctccaa atgtttcttc tctcacctct |
| 961 | tctctgggac ttaaggtgct atattccctc agagctcaca aatgcctttc aattctttcc |
| 1021 | ctgacctcct ttcctgaatt tttttatttt ctcaaatgtt acctactaag ggatgcctgg |
| 1081 | gtaagccact cagctaccta attcctcaat gacctttatc taaaatctcc atggaagcaa |
| 1141 | taaattccct tttgatgcct ctattgaatt tttcccatct ttcatctcag ggctgactga |
| 1201 | gagcataact tagaatgggc gactcttatg ttttaggcca atttcatatc attccccaga |
| 1261 | tcatatttca agtccagtaa cacaggagca accaagtaca gtgtatcctg ataatttgtt |
| 1321 | gatttcttaa ctggtgttaa tatttctttc ttccttttgt tcctaccctt ggccactgcc |
| 1381 | agccacccct caattcaggt accaacgaac cctctgccct tggctcagaa tggttatagc |
| 1441 | agaaatacaa aaaaaaaaaa aaagtctgta ctaatttcaa tatggctctt aaaaggaatg |
| 1501 | acagagaaat aggatacaag aattttgaat ctcaaaagtt atcaaaagta aaaaattttg |
| 1561 | ttaccaaaag tcaaactgca ttctcaaaac tttaaatttg tgaagaatga caacagtaga |
| 1621 | agctttcctc tccccttctc accttgagga gataaaaatt ctctaggcag gaaaagaaat |
| 1681 | ggaagccagt tagaaaaaca ttgaaataa |

Overexpression of 2 or more HLDGC genes described above can affect hair growth or density regulation and pigmentation.

DNA and Amino Acid Manipulation Methods and Purification Thereof

The present invention utilizes conventional molecular biology, microbiology, and recombinant DNA techniques available to one of ordinary skill in the art. Such techniques are well known to the skilled worker and are explained fully in the literature. See, e.g., “DNA Cloning: A Practical Approach,” Volumes 1 and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (3^{rd} ed. 2001).

One skilled in the art can obtain a protein encoded by a HLDGC gene, such as CTLA-4, IL-2, IL-21, IL-2RA/CD25, IKZF4, an HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, or a variant thereof, in several ways, which include, but are not limited to, isolating the protein via biochemical means or expressing a nucleotide sequence encoding the protein of interest by genetic engineering methods.

The invention provides for methods for using a nucleic acid encoding a HLDGC protein or variants thereof. In one embodiment, the nucleic acid is expressed in an expression cassette, for example, to achieve overexpression in a cell. The nucleic acids of the invention can be an RNA, cDNA, cDNA-like, or a DNA of interest in an expressible format, such as an expression cassette, which can be expressed from the natural promoter or an entirely heterologous promoter. The nucleic acid of interest can encode a protein, and may or may not include introns.

Protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions can be single residues, but can occur at a number of different locations at once. In one non-limiting embodiment, insertions can be on the order of about from 1 to about 10 amino acid residues, while deletions can range from about 1 to about 30 residues. Deletions or insertions can be made in adjacent pairs (for example, a deletion of about 2 residues or insertion of about 2 residues). Substitutions, deletions, insertions, or any combination thereof can be combined to arrive at a final construct. The mutations cannot place the sequence out of reading frame and should not create complementary regions that can produce secondary mRNA structure. Substitutional variants are those in which at least one residue has been removed and a different residue inserted in its place.

Substantial changes in function or immunological identity are made by selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that can produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Minor variations in the amino acid sequences of HLDGC proteins are provided by the present invention. The variations in the amino acid sequence can be when the sequence maintains at least about 30%, at least about 40%, at least about 50%, at least about 60%, at least about 70%, at least about 75%, at least about 80%, at least about 90%, at least about 95%, or at least about 99% identity to SEQ ID NO: 1, 3, 5, 7, 9, 11, 13, 15, 17, 19, 21, or 23. For example, conservative amino acid replacements can be utilized. Conservative replacements are those that take place within a family of amino acids that are related in their side chains, wherein the interchangeability of residues have similar side chains.

Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) a group of amino acids having aliphatic-hydroxyl side chains, such as serine and threonine; (ii) a group of amino acids having amide-containing side chains, such as asparagine and glutamine; (iii) a group of amino acids having aliphatic side chains such as glycine, alanine, valine, leucine, and isoleucine; (iv) a group of amino acids having aromatic side chains, such as phenylalanine, tyrosine, and tryptophan; and (v) a group of amino acids having sulfur-containing side chains, such as cysteine and methionine. Useful conservative amino acids substitution groups are: valine-leucine-isoleucine, phenylalanine-tyrosine, lysine-arginine, alanine valine, glutamic-aspartic, and asparagine-glutamine.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Bacterial and Yeast Expression Systems.

In bacterial systems, a number of expression vectors can be selected. For example, when a large quantity of a protein encoded by a Hair Loss Disorder Gene Cohort (HLDGC) gene, such as CTLA-4, IL-2, IL-21, IL-2RA/CD25, IKZF4, an HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, is needed for the induction of antibodies, vectors which direct high level expression of proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems.

If plant expression vectors are used, the expression of sequences encoding a HLDGC protein can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express HLDGC proteins. For example, in one such system Autographa californica nuclear polyhedrosis virus (AcNPV) is used as a vector to express foreign genes in Spodoptera frugiperda cells or in Trichoplusia larvae. Sequences encoding a HLDGC polypeptide can be cloned into a non-essential region of the virus, such as the polyhedrin gene, and placed under control of the polyhedrin promoter. Successful insertion of nucleic acid sequences, such as a sequence corresponding to a HLDGC gene, such as CTLA-4, IL-2, IL-21, IL-2RA/CD25, IKZF4, an HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, will render the polyhedrin gene inactive and produce recombinant virus lacking coat protein. The recombinant viruses can then be used to infect S. frugiperda cells or Trichoplusia larvae in which HLDGC or a variant thereof can be expressed.

Mammalian Expression Systems.

An expression vector can include a nucleotide sequence that encodes a HLDGC polypeptide linked to at least one regulatory sequence in a manner allowing expression of the nucleotide sequence in a host cell. A number of viral-based expression systems can be used to express a HLDGC protein or a variant thereof in mammalian host cells. For example, if an adenovirus is used as an expression vector, sequences encoding a HLDGC protein can be ligated into an adenovirus transcription/translation complex comprising the late promoter and tripartite leader sequence. Insertion into a non-essential E1 or E3 region of the viral genome can be used to obtain a viable virus which expresses a HLDGC protein in infected host cells. Transcription enhancers, such as the Rous sarcoma virus (RSV) enhancer, can also be used to increase expression in mammalian host cells.

Regulatory sequences are well known in the art, and can be selected to direct the expression of a protein or polypeptide of interest in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41) enhancers, and other expression control elements.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest is stably integrated into the genome of eukaryotic cells (for example mammalian cells, such as cells from the end bulb of the hair follicle), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, a protein encoded by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2).

Cell Transfection

A eukaryotic expression vector can be used to transfect cells in order to produce proteins encoded by nucleotide sequences of the vector. Mammalian cells (such as isolated cells from the hair bulb; for example dermal sheath cells and dermal papilla cells) can contain an expression vector (for example, one that contains a gene encoding a HLDGC protein or polypeptide) via introducing the expression vector into an appropriate host cell via methods known in the art.

A host cell strain can be chosen for its ability to modulate the expression of the inserted sequences or to process the expressed polypeptide encoded by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2 in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextran-mediated transfection, or electroporation. Electroporation is carried out at approximate voltage and capacitance to result in entry of the DNA construct(s) into cells of interest (such as cells of the end bulb of a hair follicle, for example dermal papilla cells or dermal sheath cells). Other transfection methods also include modifiedcalcium phosphate precipitation, polybrene precipitation, liposome fusion, and receptor-mediated gene delivery.

Cells that will be genetically engineered can be primary and secondary cells obtained from various tissues, and include cell types which can be maintained and propagated in culture. Non-limiting examples of primary and secondary cells include epithelial cells (for example, dermal papilla cells, hair follicle cells, inner root sheath cells, outer root sheath cells, sebaceous gland cells, epidermal matrix cells), neural cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal can be used to obtain a source of keratinocytes, fibroblasts, endothelial cells, or mesenchymal cells (for example, hair follicle cells or dermal papilla cells). In another embodiment, removal of a hair follicle can be used to obtain a source of fibroblasts, keratinocytes, endothelial cells, or mesenchymal cells (for example, hair follicle cells or dermal papilla cells). A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in United States Patent Application Publication 2004/0057937 and PCT application publication WO 2001/32840, and are hereby incorporated by reference.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein of interest (for example, a HLDGC protein or polypeptide).

Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Names, B. D., eds. (Oxford University Press: New York, 1992)). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

The mammalian cell culture that can be used with the present invention is prepared in a medium suitable for the type of cell being cultured. In one embodiment, the cell culture medium can be any one of those previously discussed (for example, MEM) that is supplemented with serum from a mammalian source (for example, fetal bovine serum (FBS)). In another embodiment, the medium can be a conditioned medium to sustain the growth of epithelial cells or cells obtained from the hair bulb of a hair follicle (such as dermal papilla cells or dermal sheath cells). For example, epithelial cells can be cultured according to Barnes and Mather in Animal Cell Culture Methods (Academic Press, 1998), which is hereby incorporated by reference in its entirety. In a further embodiment, epithelial cells or hair follicle cells can be transfected with DNA vectors containing genes that encode a polypeptide or protein of interest (for example, a HLDGC protein or polypeptide). In other embodiments of the invention, cells are grown in a suspension culture (for example, a three-dimensional culture such as a hanging drop culture) in the presence of an effective amount of enzyme, wherein the enzyme substrate is an extracellular matrix molecule in the suspension culture. For example, the enzyme can be a hyaluronidase. Epithelial cells or hair follicle cells can be cultivated according to methods practiced in the art, for example, as those described in PCT application publication WO 2004/044188 and in U.S. Patent Application Publication No. 2005/0272150, or as described by Harris in Handbook in Practical Animal Cell Biology: Epithelial Cell Culture (Cambridge Univ. Press, Great Britain; 1996; see Chapter 8), which are hereby incorporated by reference.

A suspension culture is a type of culture wherein cells, or aggregates of cells (such as aggregates of DP cells), multiply while suspended in liquid medium. A suspension culture comprising mammalian cells can be used for the maintenance of cell types that do not adhere or to enable cells to manifest specific cellular characteristics that are not seen in the adherent form. Some types of suspension cultures can include three-dimensional cultures or a hanging drop culture. A hanging-drop culture is a culture in which the material to be cultivated is inoculated into a drop of fluid attached to a flat surface (such as a coverglass, glass slide, Petri dish, flask, and the like), and can be inverted over a hollow surface. Cells in a hanging drop can aggregate toward the hanging center of a drop as a result of gravity. However, according to the methods of the invention, cells cultured in the presence of a protein that degrades the extracellular matrix (such as collagenase, chondroitinase, hyaluronidase, and the like) will become more compact and aggregated within the hanging drop culture, for degradation of the ECM will allow cells to become closer in proximity to one another since less of the ECM will be present. See also International PCT Publication No. WO2007/100870, which is incorporated by reference.

Cells obtained from the hair bulb of a hair follicle (such as dermal papilla cells or dermal sheath cells) can be cultured as a single, homogenous population (for example, comprising DP cells) in a hanging drop culture so as to generate an aggregate of DP cells. Cells can also be cultured as a heterogeneous population (for example, comprising DP and DS cells) in a hanging drop culture so as to generate a chimeric aggregate of DP and DS cells. Epithelial cells can be cultured as a monolayer to confluency as practiced in the art. Such culturing methods can be carried out essentially according to methods described in Chapter 8 of the Handbook in Practical Animal Cell Biology: Epithelial Cell Culture (Cambridge Univ. Press, Great Britain; 1996); Underhill C B, J Invest Dermatol, 1993, 101(6):820-6); in Armstrong and Armstrong, (1990) J Cell Biol 110:1439-55; or in Animal Cell Culture Methods (Academic Press, 1998), which are all hereby incorporated by reference in their entireties.

Three-dimensional cultures can be formed from agar (such as Gey's Agar), hydrogels (such as matrigel, agarose, and the like; Lee et al., (2004) Biomaterials 25: 2461-2466) or polymers that are cross-linked. These polymers can comprise natural polymers and their derivatives, synthetic polymers and their derivatives, or a combination thereof. Natural polymers can be anionic polymers, cationic polymers, amphipathic polymers, or neutral polymers. Non-limiting examples of anionic polymers can include hyaluronic acid, alginic acid (alginate), carageenan, chondroitin sulfate, dextran sulfate, and pectin. Some examples of cationic polymers, include but are not limited to, chitosan or polylysine. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73). Examples of amphipathic polymers can include, but are not limited to collagen, gelatin, fibrin, and carboxymethyl chitin. Non-limiting examples of neutral polymers can include dextran, agarose, or pullulan. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73).

Cells suitable for culturing according to methods of the invention can harbor introduced expression vectors, such as plasmids. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

Obtaining and Purifying Polypeptides

A polypeptide molecule encoded by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, or a variant thereof, can be obtained by purification from human cells expressing a HLDGC protein or polypeptide via in vitro or in vivo expression of a nucleic acid sequence encoding a HLDGC protein or polypeptide; or by direct chemical synthesis.

Detecting Polypeptide Expression.

Host cells which contain a nucleic acid encoding a HLDGC protein or polypeptide, and which subsequently express a protein encoded by a HLDGC gene, can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding a HLDGC protein or polypeptide can be detected by DNA-DNA or DNA-RNA hybridization or amplification using probes or fragments of nucleic acids encoding a HLDGC protein or polypeptide. In one embodiment, a fragment of a nucleic acid of a HLDGC gene can encompass any portion of at least about 8 consecutive nucleotides of SEQ ID NO: 2, 4, 6, 8, 10, 12, 14, 16, 18, 20, 22, or 24. In another embodiment, the fragment can comprise at least about 10 consecutive nucleotides, at least about 15 consecutive nucleotides, at least about 20 consecutive nucleotides, or at least about 30 consecutive nucleotides of SEQ ID NO: 2, 4, 6, 8, 10, 12, 14, 16, 18, 20, 22, or 24. Fragments can include all possible nucleotide lengths between about 8 and about 100 nucleotides, for example, lengths between about 15 and about 100 nucleotides, or between about 20 and about 100 nucleotides. Nucleic acid amplification-based assays involve the use of oligonucleotides selected from sequences encoding a polypeptide encoded by a HLDGC gene to detect transformants which contain a nucleic acid encoding a HLDGC protein or polypeptide.

Protocols for detecting and measuring the expression of a polypeptide encoded by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, using either polyclonal or monoclonal antibodies specific for the polypeptide are well established. Non-limiting examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a polypeptide encoded by a HLDGC gene can be used, or a competitive binding assay can be employed.

Labeling and conjugation techniques are known by those skilled in the art and can be used in various nucleic acid and amino acid assays. Methods for producing labeled hybridization or PCR probes for detecting sequences related to nucleic acid sequences encoding a HLDGC protein, such as CTLA-4, IL-2, IL-21, IL-2RA/CD25, IKZF4, a protein encoded by a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, include, but are not limited to, oligolabeling, nick translation, end-labeling, or PCR amplification using a labeled nucleotide. Alternatively, nucleic acid sequences encoding a polypeptide encoded by a HLDGC gene can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

Expression and Purification of Polypeptides.

Host cells transformed with a nucleic acid sequence encoding a HLDGC polypeptide can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a HLDGC polypeptide can be designed to contain signal sequences which direct secretion of soluble polypeptide molecules encoded by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, or a variant thereof, through a prokaryotic or eukaryotic cell membrane or which direct the membrane insertion of membrane-bound a polypeptide molecule encoded by a HLDGC gene or a variant thereof.

Other constructions can also be used to join a gene sequence encoding a HLDGC polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a polypeptide encoded by a HLDGC gene also can be used to facilitate purification. One such expression vector provides for expression of a fusion protein containing a polypeptide encoded by a HLDGC gene and 6 histidine residues preceding a thioredoxin or an enterokinase cleavage site. The histidine residues facilitate purification by immobilized metal ion affinity chromatography, while the enterokinase cleavage site provides a means for purifying the polypeptide encoded by a HLDGC gene.

A HLDGC polypeptide can be purified from any human or non-human cell which expresses the polypeptide, including those which have been transfected with expression constructs that express a HLDGC protein. A purified HLDGC protein can be separated from other compounds which normally associate with a protein encoded by a HLDGC gene in the cell, such as certain proteins, carbohydrates, or lipids, using methods practiced in the art. Non-limiting methods include size exclusion chromatography, ammonium sulfate fractionation, ion exchange chromatography, affinity chromatography, and preparative gel electrophoresis.

Chemical Synthesis.

Nucleic acid sequences comprising a HLDGC gene that encodes a polypeptide can be synthesized, in whole or in part, using chemical methods known in the art. Alternatively, a HLDGC polypeptide can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of HLDGC polypeptides can be separately synthesized and combined using chemical methods to produce a full-length molecule. In one embodiment, a fragment of a nucleic acid sequence that comprises a gene of a HLDGC can encompass any portion of at least about 8 consecutive nucleotides of SEQ ID NO: 2, 4, 6, 8, 10, 12, 14, 16, 18, 20, 22, or 24. In one embodiment, the fragment can comprise at least about 10 nucleotides, at least about 15 nucleotides, at least about 20 nucleotides, or at least about 30 nucleotides of SEQ ID NO: 2, 4, 6, 8, 10, 12, 14, 16, 18, 20, 22, or 24. Fragments include all possible nucleotide lengths between about 8 and about 100 nucleotides, for example, lengths between about 15 and about 100 nucleotides, or between about 20 and about 100 nucleotides.

A HLDGC fragment can be a fragment of a HLDGC protein, such as CTLA-4, IL-2, IL-21, IL-2RA/CD25, IKZF4, a protein encoded by a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In one embodiment, the HLA Region residing gene is selected from the group consisting of a gene of the HLA Class I Region, a gene of the HLA Class II Region, PTPN22, and AIRE. In one embodiment, the HLA Class I Region gene is HLA-A, HLA-B, HLA-C, HLA-DQB1, HLA-DRB1, MICA, MICB, HLA-G or NOTCH4. In some embodiments, the HLA Class II Region gene is HLA-DOB, HLA-DQA1, HLA-DQA2, HLA-DQB2, TAP2, or HLA-DRA. For example, the HLDGC fragment can encompass any portion of at least about 8 consecutive amino acids of SEQ ID NO: 1, 3, 5, 7, 9, 11, 13, 15, 17, 19, 21, or 23. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, or at least about 75 consecutive amino acids of SEQ ID NO: 1, 3, 5, 7, 9, 11, 13, 15, 17, 19, 21, or 23. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids.

A synthetic peptide can be substantially purified via high performance liquid chromatography (HPLC). The composition of a synthetic HLDGC polypeptide can be confirmed by amino acid analysis or sequencing. Additionally, any portion of an amino acid sequence comprising a protein encoded by a HLDGC gene can be altered during direct synthesis and/or combined using chemical methods with sequences from other proteins to produce a variant polypeptide or a fusion protein.

Identifying HLDGC Modulating Compounds.

The invention provides methods for identifying compounds which can be used for controlling and/or regulating hair growth (for example, hair density) or hair pigmentation in a subject. Since invention has provided the identification of the genes listed herein as genes associated with a hair loss disorder, the invention also provides methods for identifiying compounds that modulate the expression or activity of an HLDGC gene and/or HLDGC protein. In addition, the invention provides methods for identifying compounds which can be used for the treatment of a hair loss disorder. The invention also provides methods for identifying compounds which can be used for the treatment of hypotrichosis (for example, hereditary hypotrichosis simplex (HHS)). Non-limiting examples of hair loss disorders include: androgenetic alopecia, Alopecia areata, telogen effluvium, alopecia areata, alopecia totalis, and alopecia universalis. The methods can comprise the identification of test compounds or agents (e.g., peptides (such as antibodies or fragments thereof), small molecules, nucleic acids (such as siRNA or antisense RNA), or other agents) that can bind to a polypeptide molecule encoded by a HLDGC gene and/or have a stimulatory or inhibitory effect on the biological activity of a protein encoded by a HLDGC gene or its expression, and subsequently determining whether these compounds can regulate hair growth in a subject or can have an effect on symptoms associated with the hair loss disorders in an in vivo assay (i.e., examining an increase or reduction in hair growth).

As used herein, an “HLDGC modulating compound” refers to a compound that interacts with an HLDGC gene or an HLDGC protein or polypeptide and modulates its activity and/or its expression. The compound can either increase the activity or expression of a protein encoded by a HLDGC gene. Conversely, the compound can decrease the activity or expression of a protein encoded by a HLDGC gene. The compound can be a HLDGC agonist or a HLDGC antagonist. Some non-limiting examples of HLDGC modulating compounds include peptides (such as peptide fragments comprising a polypeptide encoded by a HLDGC gene, or antibodies or fragments thereof, fusion proteins, or the like), small molecules, and nucleic acids (such as siRNA or antisense RNA specific for a nucleic acid comprising a comprising a HLDGC). Agonists of a HLDGC protein can be molecules which, when bound to a HLDGC protein, increase or prolong the activity of the HLDGC protein. HLDGC agonists include, but are not limited to, proteins, nucleic acids, small molecules, or any other molecule which activates a HLDGC protein. Antagonists of a HLDGC protein can be molecules which, when bound to a HLDGC protein decrease the amount or the duration of the activity of the HLDGC protein. Antagonists include proteins, nucleic acids, antibodies, small molecules, or any other molecule which decrease the activity of a HLDGC protein.

The term “modulate,” as it appears herein, refers to a change in the activity or expression of a HLDGC gene or protein. For example, modulation can cause an increase or a decrease in protein activity, binding characteristics, or any other biological, functional, or immunological properties of a HLDGC protein.

In one embodiment, a HLDGC modulating compound can be a peptide fragment of a HLDGC protein that binds to the protein. For example, the HLDGC polypeptide can encompass any portion of at least about 8 consecutive amino acids of SEQ ID NO: 1, 3, 5, 7, 9, 11, 13, 15, 17, 19, 21, or 23. The fragment can comprise at least about 10 consecutive amino acids, at least about 20 consecutive amino acids, at least about 30 consecutive amino acids, at least about 40 consecutive amino acids, at least about 50 consecutive amino acids, at least about 60 consecutive amino acids, or at least about 75 consecutive amino acids of SEQ ID NO: 1, 3, 5, 7, 9, 11, 13, 15, 17, 19, 21, or 23. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The HLDGC peptide fragments can be isolated from a natural source, genetically engineered, or chemically prepared. These methods are well known in the art.

A HLDGC modulating compound can be a protein, such as an antibody (monoclonal, polyclonal, humanized, chimeric, or fully human), or a binding fragment thereof, directed against a polypeptide encoded by a HLDGC gene. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

Inhibition of RNA encoding a polypeptide encoded by a HLDGC gene can effectively modulate the expression of a HLDGC gene from which the RNA is transcribed. Inhibitors are selected from the group comprising: siRNA; interfering RNA or RNAi; dsRNA; RNA Polymerase III transcribed DNAs; ribozymes; and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a polypeptide encoded by a HLDGC gene can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area. See also, McMnaus and Sharp (2002) Nat Rev Genetics, 3:737-47, and Sen and Blau (2006) FASEB J., 20:1293-99, the entire disclosures of which are herein incorporated by reference.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector (for example, see U.S. Pat. No. 7,294,504 and U.S. Pat. No. 7,422,896, the entire disclosures of which are herein incorporated by reference). Exemplary methods for producing and testing dsRNA or siRNA molecules are described in U.S. Patent Application Publication No. 2002/0173478 to Gewirtz, U.S. Patent Application Publication No. 2007/0072204 to Hannon et al., and in U.S. Patent Application Publication No. 2004/0018176 to Reich et al., the entire disclosures of which are herein incorporated by reference.

In one embodiment, an siRNA directed to human nucleic acid sequences comprising a HLDGC gene can comprise any one of SEQ ID NOS: 41-6152. Table 10, Table 11, and Table 12 each list siRNA sequences comprising SEQ ID NOS: 41-3154, 3155-4720, and 4721-6152, respectively. In some embodiments, the siRNA is directed to SEQ ID NO: 18, 20, or a combination thereof.

RNA polymerase III transcribed DNAs contain promoters, such as the U6 promoter. These DNAs can be transcribed to produce small hairpin RNAs in the cell that can function as siRNA or linear RNAs that can function as antisense RNA. The HLDGC modulating compound can contain ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

A HLDGC modulating compound can be a small molecule that binds to a HLDGC protein and disrupts its function, or conversely, enhances its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a HLDGC protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

Knowledge of the primary sequence of a molecule of interest, such as a polypeptide encoded by a HLDGC gene, and the similarity of that sequence with proteins of known function, can provide information as to the inhibitors or antagonists of the protein of interest in addition to agonists. Identification and screening of agonists and antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as HLDGC modulating compounds, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), AMRI (Albany, N.Y.), ChemBridge (San Diego, Calif.), and MicroSource (Gaylordsville, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. For example, libraries can also include, but are not limited to, peptide-on-plasmid libraries, synthetic small molecule libraries, aptamer libraries, in vitro translation-based libraries, polysome libraries, synthetic peptide libraries, neurotransmitter libraries, and chemical libraries.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J Mol. Biol. 227:711-718; Lenstra, (1992) J. Immunol. Meth. 152:149-157; Kay et al., (1993) Gene 128:59-65; and PCT Publication No. WO 94/18318.

In vitro translation-based libraries include but are not limited to those described in PCT Publication No. WO 91/05058; and Mattheakis et al., (1994) Proc. Natl. Acad. Sci. USA 91:9022-9026.

As used herein, the term “ligand source” can be any compound library described herein, or tissue extract prepared from various organs in an organism's system, that can be used to screen for compounds that would act as an agonist or antagonist of a HLDGC protein. Screening compound libraries listed herein [also see U.S. Patent Application Publication No. 2005/0009163, which is hereby incorporated by reference in its entirety], in combination with in vivo animal studies, functional and signaling assays described below can be used to identify HLDGC modulating compounds that regulate hair growth or treat hair loss disorders.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been permethylated to generate a chemically transformed combinatorial library, is described by Ostresh et al. (1994), Proc. Natl. Acad. Sci. USA 91:11138-11142.

Computer modeling and searching technologies permit the identification of compounds, or the improvement of already identified compounds, that can modulate the expression or activity of a HLDGC protein. Having identified such a compound or composition, the active sites or regions of a HLDGC protein can be subsequently identified via examining the sites to which the compounds bind. These sites can be ligand binding sites and can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

The three dimensional geometric structure of a site, for example that of a polypeptide encoded by a HLDGC gene, can be determined by known methods in the art, such as X-ray crystallography, which can determine a complete molecular structure. Solid or liquid phase NMR can be used to determine certain intramolecular distances. Any other experimental method of structure determination can be used to obtain partial or complete geometric structures. The geometric structures can be measured with a complexed ligand, natural or artificial, which can increase the accuracy of the active site structure determined.

Other methods for preparing or identifying peptides that bind to a target are known in the art. Molecular imprinting, for instance, can be used for the de novo construction of macromolecular structures such as peptides that bind to a molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing mimics of a HLDGC modulating compound involves the steps of: (i) polymerization of functional monomers around a known substrate (the template) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

Screening Assays

HLDGC Modulating Compounds.

A HLDGC modulating compound can be a compound that affects the activity and/or expression of a HLDGC protein in vivo and/or in vitro. HLDGC modulating compounds can be agonists and antagonists of a HLDGC protein, and can be compounds that exert their effect on the activity of a HLDGC protein via the expression, via post-translational modifications, or by other means.

Test compounds or agents which bind to an HLDGC protein, and/or have a stimulatory or inhibitory effect on the activity or the expression of a HLDGC protein, can be identified by two types of assays: (a) cell-based assays which utilize cells expressing a HLDGC protein or a variant thereof on the cell surface; or (b) cell-free assays, which can make use of isolated HLDGC proteins. These assays can employ a biologically active fragment of a HLDGC protein, full-length proteins, or a fusion protein which includes all or a portion of a polypeptide encoded by a HLDGC gene). A HLDGC protein can be obtained from any suitable mammalian species (e.g., human, rat, chick, xenopus, equine, bovine or murine). The assay can be a binding assay comprising direct or indirect measurement of the binding of a test compound. The assay can also be an activity assay comprising direct or indirect measurement of the activity of a HLDGC protein. The assay can also be an expression assay comprising direct or indirect measurement of the expression of HLDGC mRNA nucleic acid sequences or a protein encoded by a HLDGC gene. The various screening assays can be combined with an in vivo assay comprising measuring the effect of the test compound on the symptoms of a hair loss disorder or disease in a subject (for example, androgenetic alopecia, alopecia areata, alopecia totalis, or alopecia universalis), loss of hair pigmentation in a subject, or even hypotrichosis.

An in vivo assay can also comprise assessing the effect of a test compound on regulating hair growth in known mammalian models that display defective or aberrant hair growth phenotypes or mammals that contain mutations in the open reading frame (ORF) of nucleic acid sequences comprising a gene of a HLDGC that affects hair growth regulation or hair density, or hair pigmentation. In one embodiment, controlling hair growth can comprise an induction of hair growth or density in the subject. Here, the compound's effect in regulating hair growth can be observed either visually via examining the organism's physical hair growth or loss, or by assessing protein or mRNA expression using methods known in the art.

Assays for screening test compounds that bind to or modulate the activity of a HLDGC protein can also be carried out. The test compound can be obtained by any suitable means, such as from conventional compound libraries. Determining the ability of the test compound to bind to a membrane-bound form of the HLDGC protein can be accomplished via coupling the test compound with a radioisotope or enzymatic label such that binding of the test compound to the cell expressing a HLDGC protein can be measured by detecting the labeled compound in a complex. For example, the test compound can be labeled with ³H, ¹⁴C, ³⁵S, or ¹²⁵I, either directly or indirectly, and the radioisotope can be subsequently detected by direct counting of radioemmission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

Cell-based assays can comprise contacting a cell expressing NKG2D with a test agent and determining the ability of the test agent to modulate (such as increase or decrease) the activity or the expression of the membrane-bound NKG2D molecule. Determining the ability of the test agent to modulate the activity of the membrane-bound NKG2D molecule can be accomplished by any method suitable for measuring the activity of such a molecule, such as monitoring downstream signaling events described in Lanier (Nat. Immunol. 2008 May; 9(5):495-502). Non-limiting examples include DAP10 phosphorylation, p85 PI3 kinase activity, Akt kinase activity, alteration in IFNγ concentration, of a NKG2D-ligand+ target cell, or a combination thereof (see also Roda-Navarro P, Reyburn H T., J Biol. Chem. 2009 Jun. 12; 284(24):16463-72; Tassi et al., Eur Immunol. 2009 April; 39(4): 1129-35; Coudert J D, et al., Blood. 2008 Apr. 1; 111(7):3571-8; Coudert J D, et al., Blood. 2005 106: 1711-1717; and Horng T, et al., Nat. Immunol. 2007 December; 8(12):1345-52, which describe methods and protocols that are all hereby incorporated by reference in their entireties).

A HLDGC protein or the target of a HLDGC protein can be immobilized to facilitate the separation of complexed from uncomplexed forms of one or both of the proteins. Binding of a test compound to a HLDGC protein or a variant thereof, or interaction of a HLDGC protein with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix (for example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtiter plates).

A HLDGC protein, or a variant thereof, can also be immobilized via being bound to a solid support. Non-limiting examples of suitable solid supports include glass or plastic slides, tissue culture plates, microtiter wells, tubes, silicon chips, or particles such as beads (including, but not limited to, latex, polystyrene, or glass beads). Any method known in the art can be used to attach a polypeptide (or polynucleotide) corresponding to HLDGC or a variant thereof, or test compound to a solid support, including use of covalent and non-covalent linkages, or passive absorption.

The diagnostic assay of the screening methods of the invention can also involve monitoring the expression of a HLDGC protein. For example, regulators of the expression of a HLDGC protein can be identified via contacting a cell with a test compound and determining the expression of a protein encoded by a HLDGC gene or HLDGC mRNA nucleic acid sequences in the cell. The expression level of a protein encoded by a HLDGC gene or HLDGC mRNA nucleic acid sequences in the cell in the presence of the test compound is compared to the protein or mRNA expression level in the absence of the test compound. The test compound can then be identified as a regulator of the expression of a HLDGC protein based on this comparison. For example, when expression of a protein encoded by a HLDGC gene or HLDGC mRNA nucleic acid sequences in the cell is statistically or significantly greater in the presence of the test compound than in its absence, the test compound is identified as a stimulator/enhancer of expression of a protein encoded by a HLDGC gene or HLDGC mRNA nucleic acid sequences in the cell. The test compound can be said to be a HLDGC modulating compound (such as an agonist).

Alternatively, when expression of a protein encoded by a HLDGC gene or HLDGC mRNA nucleic acid sequences in the cell is statistically or significantly less in the presence of the test compound than in its absence, the compound is identified as an inhibitor of the expression of a protein encoded by a HLDGC gene or HLDGC mRNA nucleic acid sequences in the cell. The test compound can also be said to be a HLDGC modulating compound (such as an antagonist). The expression level of a protein encoded by a HLDGC gene or HLDGC mRNA nucleic acid sequences in the cell in cells can be determined by methods previously described.

For binding assays, the test compound can be a small molecule which binds to and occupies the binding site of a polypeptide encoded by a HLDGC gene, or a variant thereof. This can make the ligand binding site inaccessible to substrate such that normal biological activity is prevented. Examples of such small molecules include, but are not limited to, small peptides or peptide-like molecules. In binding assays, either the test compound or a polypeptide encoded by a HLDGC gene can comprise a detectable label, such as a fluorescent, radioisotopic, chemiluminescent, or enzymatic label (for example, alkaline phosphatase, horseradish peroxidase, or luciferase). Detection of a test compound which is bound to a polypeptide encoded by a HLDGC gene can then be determined via direct counting of radioemmission, by scintillation counting, or by determining conversion of an appropriate substrate to a detectable product.

Determining the ability of a test compound to bind to a HLDGC protein also can be accomplished using real-time Biamolecular Interaction Analysis (BIA) [McConnell et al., 1992, Science 257, 1906-1912; Sjolander, Urbaniczky, 1991, Anal. Chem. 63, 2338-2345]. BIA is a technology for studying biospecific interactions in real time, without labeling any of the interactants (for example, BIA-core™). Changes in the optical phenomenon surface plasmon resonance (SPR) can be used as an indication of real-time reactions between biological molecules.

To identify other proteins which bind to or interact with a HLDGC protein and modulate its activity, a polypeptide encoded by a HLDGC gene can be used as a bait protein in a two-hybrid assay or three-hybrid assay (Szabo et al., 1995, Curr. Opin. Struct. Biol. 5, 699-705; U.S. Pat. No. 5,283,317), according to methods practiced in the art. The two-hybrid system is based on the modular nature of most transcription factors, which consist of separable DNA-binding and activation domains.

Functional Assays.

Test compounds can be tested for the ability to increase or decrease the activity of a HLDGC protein, or a variant thereof. Activity can be measured after contacting a purified HLDGC protein, a cell membrane preparation, or an intact cell with a test compound. A test compound that decreases the activity of a HLDGC protein by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for decreasing the activity of a HLDGC protein, for example an antagonist. A test compound that increases the activity of a HLDGC protein by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for increasing the activity of a HLDGC protein, for example an agonist.

Diagnosis

The invention provides methods to diagnose whether or not a subject is susceptible to or has a hair loss disorder. The diagnostic methods, in one embodiment, are based on monitoring the expression of HLDGC genes, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, in a subject, for example whether they are increased or decreased as compared to a normal sample. In one embodiment, the HLA Region residing gene is selected from the group consisting of a gene of the HLA Class I Region, a gene of the HLA Class II Region, PTPN22, and AIRE. In one embodiment, the HLA Class I Region gene is HLA-A, HLA-B, HLA-C, HLA-DQB1, HLA-DRB1MICA, MICB-, HLA-G, or NOTCH4. In one embodiment, the HLA Class II Region gene is HLA-DOB, HLA-DQA1, HLA-DQA2, HLA-DQB2, TAP2, or HLA-DRA. As used herein, the term “diagnosis” includes the detection, typing, monitoring, dosing, comparison, at various stages, including early, pre-symptomatic stages, and late stages, in adults and children. Diagnosis can include the assessment of a predisposition or risk of development, the prognosis, or the characterization of a subject to define most appropriate treatment (pharmacogenetics).

The invention provides diagnostic methods to determine whether an individual is at risk of developing a hair-loss disorder, or suffers from a hair-loss disorder, wherein the disease results from an alteration in the expression of HLDGC genes. In one embodiment, a method of detecting the presence of or a predisposition to a hair-loss disorder in a subject is provided. The subject can be a human or a child thereof. The method can comprise detecting in a sample from the subject whether or not there is an alteration in the level of expression of a protein encoded by a HLDGC gene in the subject as compared to the level of expression in a subject not afflicted with a hair-loss disorder. In one embodiment, the detecting can comprise determining whether mRNA expression of the HLDGC is increased or decreased. For example, in a microarray assay, one can look for differential expression of a HLDGC gene. Any expression of a HLDGC gene that is either 2× higher or 2× lower than HLDGC expression expression observed for a subject not afflicted with a hair-loss disorder (as indicated by a fluorescent read-out) is deemed not normal, and worthy of further investigation. The detecting can also comprise determining in the sample whether expression of at least 2 HLDGC proteins, at least 3 HLDGC proteins, at least 4 HLDGC proteins, at least 5 HLDGC proteins, at least 6 HLDGC proteins, at least 6 HLDGC proteins, at least 7 HLDGC proteins, or at least 8 HLDGC proteins is increased or decreased. The presence of such an alteration is indicative of the presence or predisposition to a hair-loss disorder.

In another embodiment, the method comprises obtaining a biological sample from a human subject and detecting the presence of a single nucleotide polymorphism (SNP) in a chromosome region containing a HLDGC gene in the subject, wherein the SNP is selected from the SNPs listed in Table 2. The SNP can comprise a single nucleotide change, or a cluster of SNPs in and around a HLDGC gene. In one embodiment, the chromosome region comprises region 2q33.2, region 4q27, region 4q31.3, region 5p13.1, region 6q25.1, region 9q31.1, region 10p15.1, region 11q13, region 12q13, region 6p21.32, or a combination thereof. In some embodiments, the single nucleotide polymorphism is selected from any one of the SNPs listed in Table 2. In further embodiments, the single nucleotide polymorphism is selected from the group consisting of rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, and rs6910071. The presence of such SNP is indicative of the presence or predisposition to a hair-loss disorder. Non-limiting examples of hair-loss disorders include androgenetic alopecia, Alopecia areata, Alopecia areata, alopecia totalis, or alopecia universalis.

The presence of an alteration in a HLDGC gene in the sample is detected through the genotyping of a sample, for example via gene sequencing, selective hybridization, amplification, gene expression analysis, or a combination thereof. In one embodiment, the sample can comprise blood, serum, sputum, lacrimal secretions, semen, vaginal secretions, fetal tissue, skin tissue, epithelial tissue, muscle tissue, amniotic fluid, or a combination thereof.

The invention provides for a diagnostic kit used to determine whether a sample from a subject exhibits increased expression of at least 2 or more HLDGC genes. In one embodiment, the kit comprising a nucleic acid primer that specifically hybridizes to one or more HLDGC genes. The invention also provides for a diagnostic kit used to determine whether a sample from a subject exhibits a predisposition to a hair-loss disorder in a human subject. In one embodiment, the kit comprises a nucleic acid primer that specifically hybridizes to a single nucleotide polymorphism (SNP) in a chromosome region containing a HLDGC gene, wherein the primer will prime a polymerase reaction only when a SNP of Table 2 is present.

In some embodiments, the primers comprise a nucleotide sequence selected from the group consisting of SEQ ID NOS: 25-40 in Table 9. In further embodiments, the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In other embodiments, the HLA Region residing gene is selected from the group consisting of a gene of the HLA Class I Region, a gene of the HLA Class II Region, PTPN22, and AIRE. In some embodiments, HLA Class I Region gene is HLA-A, HLA-B, HLA-C, HLA-DQB1, HLA-DRB1, MICA, MICB, HLA-G, or NOTCH4, while in some embodiments, the HLA Class II Region gene is HLA-DOB, HLA-DQA1, HLA-DQA2, HLA-DQB2, TAP2, or HLA-DRA.

The invention also provides a method for treating or preventing a hair-loss disorder in a subject. In one embodiment, the method comprises detecting the presence of an alteration in a HLDGC gene in a sample from the subject, the presence of the alteration being indicative of a hair-loss disorder, or the predisposition to a hair-loss disorder, and, administering to the subject in need a therapeutic treatment against a hair-loss disorder. The therapeutic treatment can be a drug administration (for example, a pharmaceutical composition comprising a siRNA directed to a HLDGC nucleic acid). In some embodiments, the siRNA is directed to ULBP3 or ULBP6. In one embodiment, the molecule comprises a polypeptide encoded by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2 comprising at least about 75%, at least about 80%, at least about 85%, at least about 90%, at least about 93%, at least about 95%, at least about 97%, at least about 98%, at least about 99%, or 100% of the amino acid sequence of SEQ ID NO: 1, 3, 5, 7, 9, 11, 13, 15, 17, 19, 21, or 23, and exhibits the function of decreasing expression of a protein encoded by a HLDGC gene. This can restore the capacity to initiate hair growth in cells derived from hair follicles or skin. In another embodiment, the molecule comprises a nucleic acid sequence comprising a HLDGC gene that encodes a polypeptide, comprising at least about 75%, at least about 80%, at least about 85%, at least about 90%, at least about 93%, at least about 95%, at least about 97%, at least about 98%, at least about 99%, or 100% of the nucleic acid sequence of SEQ ID NO: 2, 4, 6, 8, 10, 12, 14, 16, 18, 20, 22, or 24 and encodes a polypeptide with the function of decreasing expression of a protein encoded by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, thus restoring the capacity to initiate hair growth in cells derived from hair follicle cells or skin.

The alteration can be determined at the level of the DNA, RNA, or polypeptide. Optionally, detection can be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (for example, see Jones et al, (2000) Hum Genet., 106(6):663-8), or a combination thereof. In another embodiment, the detection is performed by sequencing all or part of a HLDGC gene or by selective hybridization or amplification of all or part of a HLDGC gene. A HLDGC gene specific amplification can be carried out before the alteration identification step.

An alteration in a chromosome region occupied by a gene of a HLDGC can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or non-coding portion of the gene locus, such as from two residues up to the entire gene or locus. Deletions can affect smaller regions, such as domains (introns) or repeated sequences or fragments of less than about 50 consecutive base pairs, although larger deletions can occur as well. Rearrangement includes inversion of sequences. The alteration in a chromosome region occupied by a HLDGC gene can result in amino acid substitutions, RNA splicing or processing, product instability, the creation of stop codons, frame-shift mutations, and/or truncated polypeptide production. The alteration can result in the production of a polypeptide encoded by a HLDGC gene with altered function, stability, targeting or structure. The alteration can also cause a reduction, or even an increase in protein expression. In one embodiment, the alteration in the chromosome region occupied by a gene of a HLDGC can comprise a point mutation, a deletion, or an insertion in a HLDGC gene or corresponding expression product. In another embodiment, the alteration can be a deletion or partial deletion of a HLDGC gene. The alteration can be determined at the level of the DNA, RNA, or polypeptide.

In another embodiment, the method can comprise detecting the presence of altered RNA expression. Altered RNA expression includes the presence of an altered RNA sequence, the presence of an altered RNA splicing or processing, or the presence of an altered quantity of RNA. These can be detected by various techniques known in the art, including sequencing all or part of the RNA or by selective hybridization or selective amplification of all or part of the RNA. In a further embodiment, the method can comprise detecting the presence of altered expression of a polypeptide encoded by a HLDGC gene. Altered polypeptide expression includes the presence of an altered polypeptide sequence, the presence of an altered quantity of polypeptide, or the presence of an altered tissue distribution. These can be detected by various techniques known in the art, including by sequencing and/or binding to specific ligands (such as antibodies).

Various techniques known in the art can be used to detect or quantify altered gene or RNA expression or nucleic acid sequences, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, denaturing HLPC, melting curve analysis, heteroduplex analysis, RNase protection, chemical or enzymatic mismatch cleavage, ELISA, radio-immunoassays (RIA) and immuno-enzymatic assays (IEMA). Some of these approaches (such as SSCA and CGGE) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Sequencing.

Sequencing can be carried out using techniques well known in the art, using automatic sequencers. The sequencing can be performed on the complete HLDGC gene or on specific domains thereof, such as those known or suspected to carry deleterious mutations or other alterations.

Amplification.

Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR-SSCP. Amplification usually requires the use of specific nucleic acid primers, to initiate the reaction. Nucleic acid primers useful for amplifying sequences from a HLDGC gene or locus are able to specifically hybridize with a portion of a HLDGC gene locus that flank a target region of the locus, wherein the target region is altered in certain subjects having a hair-loss disorder. In one embodiment, amplification can comprise using forward and reverse PCR primers comprising nucleotide sequences of SEQ ID NOS: 25, 27, 29, 31, 33, 35, 37, or 39, and SEQ ID NOS: 26, 28, 30, 32, 34, 36, 38, or 40, respectively (See Table 9).

The invention provides for a nucleic acid primer, wherein the primer can be complementary to and hybridize specifically to a portion of a HLDGC coding sequence (e.g., gene or RNA) altered in certain subjects having a hair-loss disorder. Primers of the invention can be specific for altered sequences in a HLDGC gene or RNA. By using such primers, the detection of an amplification product indicates the presence of an alteration in a HLDGC gene or the absence of such gene. Primers can also be used to identify single nucleotide polymorphisms (SNPs) located in or around a HLDGC gene locus; SNPs can comprise a single nucleotide change, or a cluster of SNPs in and around a HLDGC gene. Examples of primers of this invention can be single-stranded nucleic acid molecules of about 5 to 60 nucleotides in length, or about 8 to about 25 nucleotides in length. The sequence can be derived directly from the sequence of a HLDGC gene. Perfect complementarity is useful to ensure high specificity; however, certain mismatch can be tolerated. For example, a nucleic acid primer or a pair of nucleic acid primers as described above can be used in a method for detecting the presence of or a predisposition to a hair-loss disorder in a subject.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4:560, 1989; Landegren, Science 241:1077, 1988; Barringer, Gene 89:117, 1990); transcription amplification (see, e.g., Kwoh, Proc. Natl. Acad. Sci. USA 86:1173, 1989); and, self-sustained sequence replication (see, e.g., Guatelli, Proc. Natl. Acad. Sci. USA 87:1874, 1990); Q Beta replicase amplification (see, e.g., Smith, J. Clin. Microbiol. 35:1477-1491, 1997), automated Q-beta replicase amplification assay (see, e.g., Burg, Mol. Cell. Probes 10:257-271, 1996) and other RNA polymerase mediated techniques (e.g., NASBA, Cangene, Mississauga, Ontario); see also Berger, Methods Enzymol. 152:307-316, 1987; Sambrook; Ausubel; U.S. Pat. Nos. 4,683,195 and 4,683,202; Sooknanan, Biotechnology 13:563-564, 1995. All the references stated above, an throughout the description, are incorporated by reference in their entireties.

Selective Hybridization.

Hybridization detection methods are based on the formation of specific hybrids between complementary nucleic acid sequences that serve to detect nucleic acid sequence alteration(s). A detection technique involves the use of a nucleic acid probe specific for wild type or altered gene or RNA, followed by the detection of the presence of a hybrid. The probe can be in suspension or immobilized on a substrate or support (for example, as in nucleic acid array or chips technologies). The probe can be labeled to facilitate detection of hybrids. For example, a sample from the subject can be contacted with a nucleic acid probe specific for a wild type HLDGC gene or an altered HLDGC gene, and the formation of a hybrid can be subsequently assessed. In one embodiment, the method comprises contacting simultaneously the sample with a set of probes that are specific, respectively, for a wild type HLDGC gene and for various altered forms thereof. Thus, it is possible to detect directly the presence of various forms of alterations in a HLDGC gene in the sample. Also, various samples from various subjects can be treated in parallel.

According to the invention, a probe can be a polynucleotide sequence which is complementary to and can specifically hybridize with a (target portion of a) HLDGC gene or RNA, and that is suitable for detecting polynucleotide polymorphisms associated with alleles of a HLDGC gene (or genes) which predispose to or are associated with a hair-loss disorder. Useful probes are those that are complementary to a HLDGC gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a HLDGC gene or RNA that carries an alteration. For example, the probe can be directed to a chromosome region occupied by a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2. In one embodiment, the HLA Region residing gene is selected from the group consisting of a gene of the HLA Class I Region, a gene of the HLA Class II Region, PTPN22, and AIRE. In one embodiment, the HLA Class I Region gene is HLA-A, HLA-B, HLA-C, HLA-DQB1, HLA-DRB1, MICA, MICB, HLA-G, or NOTCH4. In one embodiment, the HLA Class II Region gene is HLA-DOB, HLA-DQA1, HLA-DQA2, HLA-DQB2, TAP2, or HLA-DRA. In one embodiment, the chromosome region comprises region 2q33.2, region 4q27, region 4q31.3, region 5p13.1, region 6q25.1, region 9q31.1, region 10p15.1, region 11q13, region 12q13, region 6p21.32, or a combination thereof.

The sequence of the probes can be derived from the sequences of a HLDGC gene and RNA as provided herein. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in e.g., Sambrook, ed., Molecular Cloning: A Laboratory Manual (3^{rd} Ed.), Vols. 1-3, Cold Spring Harbor Laboratory, 2001; Current Protocols in Molecular Biology, Ausubel, ed. John Wiley & Sons, Inc., New York, 1997; Laboratory Techniques in Biochemistry and Molecular Biology: Hybridization with Nucleic Acid Probes, Part I. Theory and Nucleic Acid Preparation, Tijssen, ed. Elsevier, N.Y., 1993.

DNA Microarrays.

An approach to detecting gene expression or nucleotide variation involves using nucleic acid arrays placed on chips. This technology has been exploited by companies such as Affymetrix and Illumina, and a large number of technologies are commercially available (see also the following reviews: Grant and Hakonarson, 2008, Clinical Chemistry, 54(7): 1116-1124; Curtis et al., 2009, BMC Genomics, 10:588; and Syvänen, 2005, Nature Genetics, 37:S5-S10, each of which are hereby incorporated by reference in their entireties). Useful array technologies include, but are not limited to, chip-based DNA technologies such as those described by Hacia et al. (Nature Genet., 14:441-449, 1996) and Shoemaker et al. (Nature Genetics, 14:450-456, 1996). These techniques involve quantitative methods for analyzing large numbers of sequences rapidly and accurately (see Erdogan et al., 2001, Nuc Acids Res, 29(7):e36 and Bier et al., 2008, Adv. Biochem Engin/Biotechnol, 109:433-453, each of which are hereby incorporated by reference in their entireties). The technology exploits the complementary binding properties of single stranded DNA to screen DNA samples by hybridization (Pease et al., Proc. Natl. Acad. Sci. USA, 91:5022-5026, 1994; Fodor et al., Science, 251:767-773, 1991).

A microarray or gene chip can comprise a solid substrate to which an array of single-stranded DNA molecules has been attached. For screening, the chip or microarray is contacted with a single-stranded DNA sample, which is allowed to hybridize under stringent conditions. The chip or microarray is then scanned to determine which probes have hybridized. For example see methods discussed in Bier et al., 2008, Adv. Biochem Engin/Biotechnol, 109:433-453. In a some embodiments, a chip or microarray can comprise probes specific for SNPs evidencing the predisposition towards the development of a hairloss disorder. Such probes can include PCR products amplified from patient DNA synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change. In some embodiments, the cDNA- or oligonucleotide-microarray comprises SEQ ID NOS: 2, 4, 6, 8, 10, 12, 14, 16, 18, 20, 22, 24, or a combination thereof. In other embodiments, the cDNA- or oligonucleotide-microarray comprises SNPs listed in Table 2. In further embodiments, the cDNA- or oligonucleotide-microarray comprises SNPs rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, or rs6910071.

Gene chip or microarray formats are described in the art, for example U.S. Pat. Nos. 5,861,242 and 5,578,832, which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of nucleic acid probes; (3) a sampling element; (4) a detector; and (5) a signal amplification/treatment system. A chip may also include a support for immobilizing the probe.

Arrays of nucleic acids can be generated by any number of known methods including photolithography, pipette, drop-touch, piezoelectric, spotting, and electric procedures. The DNA microarrays generally have probes that are supported by a substrate so that a target sample is bound or hybridized with the probes. In use, the microarray surface is contacted with one or more target samples under conditions that promote specific, high-affinity binding of the target to one or more of the probes. A sample solution containing the target sample can comprise fluorescently, radioactive, or chemoluminescently labeled molecules that are detectable. The hybridized targets and probes can also be detected by voltage, current, or electronic means known in the art.

Various techniques can be used to prepare an oligonucleotide for use in a microarray. In situ synthesis of oligonucleotide or polynucleotide probes on a substrate can be performed according to chemical processes known in the art, such as sequential addition of nucleotide phosphoramidites to surface-linked hydroxyl groups. Indirect synthesis may also be performed via biosynthetic techniques such as PCR. Other methods of oligonucleotide synthesis include phosphotriester and phosphodiester methods and synthesis on a support, as well as phosphoramidate techniques. Chemical synthesis via a photolithographic method of spatially addressable arrays of oligonucleotides bound to a substrate made of glass can also be employed.

The probes or oligonucleotides can be obtained by biological synthesis or by chemical synthesis. Chemical synthesis allows for low molecular weight compounds and/or modified bases to be incorporated during specific synthesis steps. Furthermore, chemical synthesis is very flexible in the choice of length and region of target polynucleotides binding sequence. The oligonucleotide can be synthesized by standard methods such as those used in commercial automated nucleic acid synthesizers.

For example, probes or oligonucleotides may be directly or indirectly immobilized onto a surface to ensure optimal contact and maximum detection. The ability to directly synthesize on or attach polynucleotide probes to solid substrates is well known in the art; for example, see U.S. Pat. Nos. 5,837,832 and 5,837,860, both of which are expressly incorporated by reference.

A variety of methods have been utilized to either permanently or removably attach probes or oligonucleotides to the substrate. Exemplary methods include: the immobilization of biotinylated nucleic acid molecules to avidin/streptavidin coated supports (Holmstrom, Anal. Biochem. 209:278-283, 1993), the direct covalent attachment of short, 5′-phosphorylated primers to chemically modified polystyrene plates (Rasmussen et al., Anal. Biochem, 198:138-142, 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., BioTechniques 8:276-277, 1990; Newton et al., Nucl. Acids Res. 21:1155-1162, 1993).

When immobilized onto a substrate, the probes or oligonucleotides are stabilized and therefore may be used repeatedly. Hybridization is performed on an immobilized nucleic acid that is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), and photopolymers (which contain photoreactive species such as nitrenes, carbenes and ketyl radicals) that can form covalent links with target. molecules.

Binding of the probes or oligonucleotides to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA probes or oligonucleotides may be bound directly to membranes using ultraviolet radiation. With nitrocellose membranes, the DNA probes or oligonucleotides are spotted onto the membranes. A UV light source (Stratalinker™, Stratagene, La Jolla, Calif.) is used to irradiate DNA spots and induce cross-linking. An alternative method for cross-linking involves baking the spotted membranes at 80° C. for two hours in vacuum.

Specific DNA probes or oligonucleotides can first be immobilized onto a membrane and then attached to a membrane in contact with a transducer detection surface. This method avoids binding the probe onto the transducer and may be desirable for large-scale production. Membranes suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

Specific Ligand Binding.

As discussed herein, alteration in a chromosome region occupied by a HLDGC gene or alteration in expression of a HLDGC gene, such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2, can also be detected by screening for alteration(s) in a sequence or expression level of a polypeptide encoded by a HLDGC gene. Different types of ligands can be used, such as specific antibodies. In one embodiment, the sample is contacted with an antibody specific for a polypeptide encoded by a HLDGC gene and the formation of an immune complex is subsequently determined. Various methods for detecting an immune complex can be used, such as ELISA, radioimmunoassays (RIA) and immuno-enzymatic assays (IEMA).

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a polypeptide encoded by a HLDGC gene (such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2) can be an antibody that selectively binds such a polypeptide, namely, an antibody raised against a polypeptide encoded by a HLDGC gene or an epitope-containing fragment thereof. Although non-specific binding towards other antigens can occur, binding to the target polypeptide occurs with a higher affinity and can be reliably discriminated from non-specific binding. In one embodiment, the method can comprise contacting a sample from the subject with an antibody specific for a wild type or an altered form of a polypeptide encoded by a HLDGC gene, and determining the presence of an immune complex. Optionally, the sample can be contacted to a support coated with antibody specific for the wild type or altered form of a polypeptide encoded by a HLDGC gene. In one embodiment, the sample can be contacted simultaneously, or in parallel, or sequentially, with various antibodies specific for different forms of a polypeptide encoded by a HLDGC gene, such as a wild type and various altered forms thereof.

As discussed herein, the invention also provides for a diagnostic kit comprising products and reagents for detecting in a sample obtained from a subject the presence of an alteration in one or more HLDGC genes or polypeptides thereof, the expression of one or more HLDGC genes or polypeptide thereof, the presence of a HLDGC-specific SNP (for example, those SNPs listed in Table 2), and/or the activity of one or more HLDGC genes. The kit can be useful for determining whether a sample from a subject exhibits reduced expression of a HLDGC gene or of a protein encoded by a HLDGC gene, or exhibits a deletion or alteration in one or more HLDGC genes. For example, the diagnostic kit according to the present invention comprises any primer, any pair of primers, any nucleic acid probe and/or any ligand, (for example, an antibody directed against polypeptides encoded by HLDGC gene(s)), described in the present invention. The diagnostic kit according to the present invention can further comprise reagents and/or protocols for performing a hybridization, amplification or antigen-antibody immune reaction. In one embodiment, the kit can comprise nucleic acid primers that specifically hybridize to and can prime a polymerase reaction from nucleic acid sequences comprising a gene of a HLDGC that encode a polypeptide of such. In another embodiment, the primer comprises any one of the nucleotide sequences of Table 9.

The diagnosis methods can be performed in vitro, ex vivo, or in vivo, using a sample from the subject, to assess the status of a chromosome region occupied by a gene of the HLDGC. The sample can be any biological sample derived from a subject, which contains nucleic acids or polypeptides. Examples of such samples include, but are not limited to, fluids, tissues, cell samples, organs, or tissue biopsies. Non-limiting examples of samples include blood, plasma, saliva, urine, or seminal fluid. Pre-natal diagnosis can also be performed by testing fetal cells or placental cells, for instance. Screening of parental samples can also be used to determine risk/likelihood of offspring possessing the germline mutation. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), centrifugation. Also, the nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents such as probes, primers, or ligands in order to assess the presence of an altered chromosome region occupied by a HLDGC gene or the presence of a HLDGC-specific SNP (for example, those SNPs listed in Table 2). Contacting can be performed in any suitable device, such as a plate, tube, well, array chip, or glass. In specific embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

Identifying an altered polypeptide, RNA, or DNA in the sample is indicative of the presence of an altered HLDGC gene (such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2) in the subject, which can be correlated to the presence, predisposition or stage of progression of a hair-loss disorder. For example, an individual having a germ line mutation has an increased risk of developing a hair-loss disorder. The determination of the presence of an altered chromosome region occupied by a gene of a HLDGC in a subject also allows the design of appropriate therapeutic intervention, which is more effective and customized. Also, this determination at the pre-symptomatic level allows a preventive regimen to be applied.

Gene Therapy and Protein Replacement Methods

Delivery of nucleic acids into viable cells can be effected ex vivo, in situ, or in vivo by use of vectors, such as viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (See, for example, Anderson, Nature, supplement to vol. 392, no. 6679, pp. 25-20 (1998)). Introduction of a nucleic acid or a gene encoding a polypeptide of the invention can also be accomplished with extrachromosomal substrates (transient expression) or artificial chromosomes (stable expression). Cells may also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., 1992), adenovirus (Berkner, 1992; Berkner et al., 1988; Gorziglia and Kapikian, 1992; Quantin et al., 1992; Rosenfeld et al., 1992; Wilkinson et al., 1992; Stratford-Perricaudet et al., 1990), vaccinia virus (Moss, 1992), adeno-associated virus (Muzyczka, 1992; Ohi et al., 1990), herpesviruses including HSV and EBV (Margolskee, 1992; Johnson et al., 1992; Fink et al., 1992; Breakfield and Geller, 1987; Freese et al., 1990), and retroviruses of avian (Biandyopadhyay and Temin, 1984; Petropoulos et al., 1992), murine (Miller, 1992; Miller et al., 1985; Sorge et al., 1984; Mann and Baltimore, 1985; Miller et al., 1988), and human origin (Shimada et al., 1991; Helseth et al., 1990; Page et al., 1990; Buchschacher and Panganiban, 1992). Non-limiting examples of in vivo gene transfer techniques include transfection with viral (e.g., retroviral) vectors (see U.S. Pat. No. 5,252,479, which is incorporated by reference in its entirety) and viral coat protein-liposome mediated transfection (Dzau et al., Trends in Biotechnology 11:205-210 (1993), incorporated entirely by reference). For example, naked DNA vaccines are generally known in the art; see Brower, Nature Biotechnology, 16:1304-1305 (1998), which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al., Science 256:808-813 (1992); U.S. Pat. Nos. 5,252,479, 5,747,469, 6,017,524, 6,143,290, 6,410,010 6,511,847; and U.S. Application Publication Nos. 2002/0077313 and 2002/00069, which are all hereby incorporated by reference in their entireties. For additional reviews of gene therapy technology, see Friedmann, Science, 244:1275-1281 (1989); Verma, Scientific American: 68-84 (1990); Miller, Nature, 357: 455-460 (1992); Kikuchi et al., J Dermatol Sci. 2008 May; 50(2):87-98; Isaka et al., Expert Opin Drug Deliv. 2007 September; 4(5):561-71; Jager et al., Curr Gene Ther. 2007 August; 7(4):272-83; Waehler et al., Nat Rev Genet. 2007 August; 8(8):573-87; Jensen et al., Ann Med. 2007; 39(2):108-15; Herweijer et al., Gene Ther. 2007 January; 14(2):99-107; Eliyahu et al., Molecules, 2005 Jan. 31; 10(1):34-64; and Altaras et al., Adv Biochem Eng Biotechnol. 2005; 99:193-260, all of which are hereby incorporated by reference in their entireties.

Protein replacement therapy can increase the amount of protein by exogenously introducing wild-type or biologically functional protein by way of infusion. A replacement polypeptide can be synthesized according to known chemical techniques or may be produced and purified via known molecular biological techniques. Protein replacement therapy has been developed for various disorders. For example, a wild-type protein can be purified from a recombinant cellular expression system (e.g., mammalian cells or insect cells-see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

A polypeptide encoded by an HLDGC gene (for example, CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2) can also be delivered in a controlled release system. For example, the polypeptide may be administered using intravenous infusion, an implantable osmotic pump, a transdermal patch, liposomes, or other modes of administration. In one embodiment, a pump may be used (see is Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

Pharmaceutical Compositions and Administration for Therapy

HLDGC proteins and HLDGC modulating compounds of the invention can be administered to the subject once (e.g., as a single injection or deposition). Alternatively, HLDGC proteins and HLDGC modulating compounds can be administered once or twice daily to a subject in need thereof for a period of from about two to about twenty-eight days, or from about seven to about ten days. HLDGC proteins and HLDGC modulating compounds can also be administered once or twice daily to a subject for a period of 1, 2, 3, 4, 5, 6, 7, 8, 9, 10, 11, 12 times per year, or a combination thereof. Furthermore, HLDGC proteins and HLDGC modulating compounds of the invention can be co-administrated with another therapeutic. Where a dosage regimen comprises multiple administrations, the effective amount of the HLDGC proteins and HLDGC modulating compounds administered to the subject can comprise the total amount of gene product administered over the entire dosage regimen.

HLDGC proteins and HLDGC modulating compounds can be administered to a subject by any means suitable for delivering the HLDGC proteins and HLDGC modulating compounds to cells of the subject, such as the dermis, epidermis, dermal papilla cells, or hair follicle cells. For example, HLDGC proteins and HLDGC modulating compounds can be administered by methods suitable to transfect cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with a hair-loss disorder by any means that produces contact of the active ingredient with the agent's site of action in the body of a subject, such as a human or animal (e.g., a dog, cat, or horse). They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

A therapeutically effective dose of HLDGC modulating compounds can depend upon a number of factors known to those or ordinary skill in the art. The dose(s) of the HLDGC modulating compounds can vary, for example, depending upon the identity, size, and condition of the subject or sample being treated, further depending upon the route by which the composition is to be administered, if applicable, and the effect which the practitioner desires the HLDGC modulating compounds to have upon the nucleic acid or polypeptide of the invention. These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa. (20^{th} Ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

The invention also provides for a kit that comprises a pharmaceutically acceptable carrier and a HLDGC modulating compound identified using the screening assays of the invention packaged with instructions for use. For modulators that are antagonists of the activity of a HLDGC protein, or which reduce the expression of a HLDGC protein, the instructions would specify use of the pharmaceutical composition for promoting the loss of hair on the body surface of a mammal (for example, arms, legs, bikini area, face).

For HLDGC modulating compounds that are agonists of the activity of a HLDGC protein or increase the expression of one or more proteins encoded by HLDGC genes (such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2), the instructions would specify use of the pharmaceutical composition for regulating hair growth. In one embodiment, the instructions would specify use of the pharmaceutical composition for the treatment of hair loss disorders. In a further embodiment, the instructions would specify use of the pharmaceutical composition for restoring hair pigmentation. For example, administering an agonist can reduce hair graying in a subject.

A pharmaceutical composition containing a HLDGC modulating compound can be administered in conjunction with a pharmaceutically acceptable carrier, for any of the therapeutic effects discussed herein. Such pharmaceutical compositions can comprise, for example antibodies directed to polypeptides encoded by genes comprising a HLDGC or variants thereof, or agonists and antagonists of a polypeptide encoded by a HLDGC gene. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of injectable compositions can be brought about by incorporating an agent which delays absorption, for example, aluminum monostearate and gelatin.

Sterile injectable solutions can be prepared by incorporating the HLDGC modulating compound (e.g., a polypeptide or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art

In some embodiments, the HLDGC modulating compound can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Various routes of administration and various sites of cell implantation can be utilized, such as, subcutaneous or intramuscular, in order to introduce the aggregated population of cells into a site of preference. Once implanted in a subject (such as a mouse, rat, or human), the aggregated cells can then stimulate the formation of a hair follicle and the subsequent growth of a hair structure at the site of introduction. In another embodiment, transfected cells (for example, cells expressing a protein encoded by a HLDGC gene (such as CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2) are implanted in a subject to promote the formation of hair follicles within the subject. In further embodiments, the transfected cells are cells derived from the end bulb of a hair follicle (such as dermal papilla cells or dermal sheath cells). Aggregated cells (for example, cells grown in a hanging drop culture) or transfected cells (for example, cells produced as described herein) maintained for 1 or more passages can be introduced (or implanted) into a subject (such as a rat, mouse, dog, cat, human, and the like).

“Subcutaneous” administration can refer to administration just beneath the skin (i.e., beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration.

This mode of administration can be feasible where the subcutaneous layer is sufficiently thin so that the factors present in the compositions can migrate or diffuse from the locus of administration and contact the hair follicle cells responsible for hair formation. Thus, where intradermal administration is utilized, the bolus of composition administered is localized proximate to the subcutaneous layer.

Administration of the cell aggregates (such as DP or DS aggregates) is not restricted to a single route, but may encompass administration by multiple routes. For instance, exemplary administrations by multiple routes include, among others, a combination of intradermal and intramuscular administration, or intradermal and subcutaneous administration. Multiple administrations may be sequential or concurrent. Other modes of application by multiple routes will be apparent to the skilled artisan.

In other embodiments, this implantation method will be a one-time treatment for some subjects. In further embodiments of the invention, multiple cell therapy implantations will be required. In some embodiments, the cells used for implantation will generally be subject-specific genetically engineered cells. In another embodiment, cells obtained from a different species or another individual of the same species can be used. Thus, using such cells may require administering an immunosuppressant to prevent rejection of the implanted cells. Such methods have also been described in United States Patent Application Publication 2004/0057937 and PCT application publication WO 2001/32840, and are hereby incorporated by reference.

These methods described herein are by no means all-inclusive, and further methods to suit the specific application is understood by the ordinary skilled artisan. Moreover, the effective amount of the compositions can be further approximated through analogy to compounds known to exert the desired effect.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Genomewide Association Study in Alopecia Areata Implicates Both Innate and Adaptive Immunity

We undertook a genome-wide association study (GWAS) in an initial discovery sample of 250 unrelated cases and 1049 controls, and replicated our findings in an independent sample of 804 cases and 2229 controls.

Joint analysis of the datasets identified 141 SNPs that are significantly associated with AA (p≦5×10⁻⁷). We identified association with several key components of Treg activation and proliferation, CTLA4, IL-2/IL-21, IL-2RA/CD25, and Eos (IKZF4), as well as the HLA class II region. We also found evidence for genes expressed in the hair follicle itself (PTGER4, PRDX5, STX17). Unexpectedly, a region of strong association resides within the ULBP gene cluster on chromosome 6q25.1, encoding activating ligands of the natural killer cell receptor, NKG2D, which have never before been implicated in an autoimmune disease. We discovered that expression of ULBP3 in lesional scalp from AA patients is markedly upregulated in the hair follicle dermal sheath during active disease.

This study provides evidence for involvement of both innate and acquired immunity in the pathogenesis of AA. Taken together, we have defined the genetic underpinnings of AA for the first time, placing AA within the context of shared pathways among autoimmune diseases, and implicating a new disease mechanism, the upregulation of ULBP ligands, in triggering autoimmunity.

The concept of an early ‘danger signal’ emanating from the hair follicle can be a key initial event in triggering the cascade of AA immunopathogenesis.^{N4} Evidence supporting a genetic basis for AA stems from multiple lines of evidence, including the observed heritability in first degree relatives,^{N5,N6} twin studies,^{N7} and most recently, from the results of our family-based linkage studies.^{N8} A number of candidate-gene association studies have been performed, mainly by selecting genes implicated in other autoimmune diseases, (reviewed in^{N3}), however, these studies were both underpowered in terms of sample size and by definition, biased by choices of candidate genes. Specifically, associations have been reported for HLA-residing genes (HLA-DQB1, HLA-DRB1, HLA-B, HLA-C, NOTCH4, MICA), as well as genes outside of the HLA (PTPN22, AIRE).

To determine the genetic basis of AA using an unbiased approach, in this study we performed a GWAS 1055 AA cases and 3278 controls, and identified 141 SNPs that exceeded genome-wide significance (p≦5×10⁻⁷). Unexpectedly, we found evidence for genes involved in both the innate and adaptive immune responses, as well as upregulation of ‘danger signals’ in affected hair follicles that contribute to disease pathogenesis.

Methods

Patient Population.

Cases were ascertained through the National Alopecia Areata Registry (NAAR)^{N9} with approval from institutional review boards, which recruits patients in the US primarily through five clinical sites. Three sets of previously published control datasets were used for comparison of allele frequencies.^{N10-N12} All samples were genotyped on the Illumina HumanHap 550v2 or 610 chip and were confirmed to be of European ancestry by principal component analysis with ancestry informative markers. Stringent quality control measures were used to remove samples and markers that did not exceed pre-defined thresholds. Tests of association were run with and without measures to control for residual population stratification. Tissue specimens and RNA from human scalp biopsies were obtained with approval from institutional review boards. All experiments were performed according to the Helsinki guidelines.

Genotyping.

Quality control was performed with Helix Tree software (Golden Helix) or PLINK (http://pngu.mgh.harvard.edu/purcell/plink/)^{N33}. SNPs that were missing more than 5% data, did not follow Hardy Weinberg Equilibrium in controls (p<0.0001), or were not present in both Illumina 550 Kv2 and Illumina 610K were removed, leaving 463, 308 SNPs for analysis. Next, 19 samples with more than 10% missing genotype data were removed. In addition, 3 case and 8 control samples that shared more than 25% inferred identity by descent were removed. Principal component analysis (PCA) using a subset of 3568 ancestry informative markers^{N34} (AIMs) identified 5 cases and 12 controls as ethnic outliers and removed prior to analysis. Samples more than 6 standard deviations units from 5 components were excluded from subsequent analysis. Visual inspection of a plot of the first two eigenvectors identified 141 controls for which matched cases did not exist. These were excluded from further analysis.

Statistical Analysis.

Reported association values were obtained with logistic regression assuming an additive genetic model and included a covariate to adjust for any residual population stratification. Statistics unadjusted for residual population stratification were also examined, as well as p-values obtained with the false discovery rate method and were found to be equivalent to reported values. LD was quantitated and evaluated with Haploview^{N35}. SAS was used to perform stratified analysis and logistic modeling to determine if SNPs shared a common haplotype. If the adjusted OR differed from the crude estimate by more than 10%, then a common haplotype was inferred. Assessment of individual genetic liability was performed in Excel (Microsoft). A single marker was chosen as a proxy for each of the independent risk haplotypes. Alleles for the 18 proxy markers were coded 1 if associated with increased risk and 0 otherwise, and then summed for each individual. A two-tailed student t-test was used to determine the significance of the difference in the distribution of risk alleles between cases and controls, under an assumption of unequal variance. The population attributable fraction (AFₚ) for each SNP was calculated as

$${AF}_{p} = \frac{PF ( OR - 1 )}{1 + PF ( OR - 1 )}$$

where ORᵢ indexes the estimate associated with heterozygous and homozygous carriage of risk-increasing genotypes, and PFᵢ denotes the genotype frequencies in the controls. LD-based imputation using the Markov Chain Haplotyping algorithm (MACH 1.0.16, http://www.sph.umich.edu/csg/abecasis/mach/tour/imputation.html) was used to carry out genome-wide maximum likelihood genotype imputation. Weighted logistic regression test on binary trait using mach2dat was used to assess the quality of the imputation, again followed by logistic regression association test assuming an additive model with top 10 principle components as covariates to adjust for any residual population stratification using PLINK.

Tissue specimens.

Human skin scalp biopsies were obtained from 19 AA patients (age range 28-77 years) from a lesional area, while control samples were either frontotemporal human skin scalp biopsies taken from seven healthy women undergoing facelift surgery (age range 35-67 years), or occipital region of human skin scalp biopsies from two healthy men. All experiments were performed according to the Helsinki guidelines. Specimens were embedded directly in OCT compound, or fixed in 10% formalin and embedded in paraffin blocks and cut into 5 μm-thick sections.

Immunohistology.

In order to detect ULBP3 protein expression in situ a labeled-streptavidin-biotin-method (LSAB)-based staining was performed. Briefly, paraffin sections were deparaffinised and immunostained after antigen retrieval with citrate buffer, and appropriate blocking steps against endogenous peroxidase, using the rabbit antihuman ULBP3 antibody (1:250 in antibody diluent, DCS, Hamburg, Germany) overnight at 4° C. All incubation steps were interspersed by washing with Tris-buffered saline (TBS, 0.05 M, pH 7.6; 3×5 min). This was followed by staining with a biotinylated PolyLink secondary antibody (DCS) for 20 min at RT, and developed using the peroxidase-streptavidin-conjugate (DCS, 20 min at RT) method. Finally, the slides were labelled with 3-amino-9-ethylcarbazole (AEC) substrate (Vector Elite ABC Kit, Vector Laboratories, Burlingame, USA) and counterstained with haematoxylin.

Quantitative Immunohistomorphometry.

The number of ULBP3 positive cells was evaluated in 3 microscopic fields at 200 times magnification in the dermis, and in the hair follicle (HF) connective tissue sheath (CTS) and parafollicular around each hair bulb of AA and control skin. All data were analyzed by Mann-Whitney-Test for unpaired samples (expressed as mean±SEM; p values of <0.05 regarded as significant).

Indirect Immunofluorescence (IIF).

IIF on fresh frozen sections of human scalp skin was performed as described previously.^{N36} The primary antibodies used were mouse monoclonal anti-ULBP3 (clone 2F9; diluted 1:50; Santa Cruz Biotechnology), rabbit polyclonal anti-CD3 (1:50; DAKO), mouse monoclonal anti-CD8 (clone C8/144B; prediluted; Abcam), rabbit polyclonal anti-CD8 (1:200; Abcam), mouse monoclonal anti-NKG2D (clone 1D11; 1:100; Abcam), rabbit polyclonal anti-PTGER4 (1:25; Sigma), rabbit polyclonal anti-STX17 (1:500; Sigma), rabbit polyclonal anti-PRDX5 (1:500; Abnova), guinea pig polyclonal anti-K74 (1:2,000), and guinea pig polyclonal anti-K31 (1:8,000). The anti-K74 and anti-K31 antibodies were kindly provided by Dr. Lutz Langbein in German Cancer Research Center.

RT-PCR Analysis.

Total RNA was isolated from scalp skin and whole blood of a healthy control individual using the RNeasy® Minikit according to the manufacturer's instructions (Qiagen). 2 μg of total RNA was reverse-transcribed using oligo-dT primers and SuperScript™ III (Invitrogen). Using the first-strand cDNAs as templates, PCR was performed using Platinum® PCR SuperMix (Invitrogen) and primer pairs shown in Table 9. The amplification conditions were 94° C. for 2 min, followed by 35 cycles of 94° C. for 30 sec, 60° C. for 30 sec, and 72° C. for 50 sec, with a final extension at 72° C. for 7 min. PCR products were run on 2.0% agarose gels. Real-time PCR was performed on an ABI 7300 (Applied Biosystems). PCR reactions were performed using ABI SYBR Green PCR Master Mix, 300 nM primers, 50 ng cDNA at the following consecutive steps: (a) 50° C. for 2 min, (b) 95° C. for 10 min, (c) 40 cycles of 95° C. for 15 sec and 60° C. for 1 min. The samples were run in triplicate and normalized to an internal control (GAPDH) using the accompanying software.

TABLE 9
Primer Sequences.

| | | SEQ | | SEQ | product |
|---|---|---|---|---|---|
| | forward primer | ID | reverse primer | ID | size |
| gene | (5′ to 3′) | NO: | (5′ to 3′) | NO: | (bp) |
| ULBP3 | GATTTCACACCCA | 25 | CTATGGCTTTGG | 26 | 337 |
| | GTGGACC | | GTTGAGCTAA | | |
| STX17 | TCCATGACTGTTG | 27 | CTCCTGCTGAGA | 28 | 192 |
| | GTGGAGCA | | ATTCACTAGG | | |
| PRDX5 | TCGCTGGTGTCCA | 29 | TGGCCAACATTCC | 30 | 230 |
| | TCTTTGG | | AATTGCAG | | |
| PTGER4 | CGAGATCCAGATG | 31 | GGTCTAGGATGG | 32 | 179 |
| | GTCATCTTAC | | GGTTCACA | | |
| IKZF4 | CTCACCGGCAAGG | 33 | GATGAGTCCCCG | 34 | 133 |
| | GAAGGAT | | CTACTTTCA | | |
| IL2RA | TGGCAGCGGAGAC | 35 | ACGCAGGCAAGC | 36 | 163 |
| | AGAGGAA | | ACAACGGA | | |
| KRT15 | GGGTTTTGGTGGT | 37 | TCGTGGTTCTTCT | 38 | 474 |
| | GGCTTTG | | TCAGGTAGGC | | |
| GAPDH | TCACCAGGGCTGC | 39 | GGGTGGAATCAT | 40 | 105 |
| | TTTTAACTC | | ATTGGAACATG | | |

TABLE 10
Human NKG2D NM_007360

| SEQ | | SEQ | |
|---|---|---|---|
| ID | | ID | |
| NO. | siRNA (19 bp) | NO. | Reverse complement |
| 41 | ACTTTCAATTCTAGATCAG | 42 | CTGATCTAGAATTGAAAGT |
| 43 | CTTTCAATTCTAGATCAGG | 44 | CCTGATCTAGAATTGAAAG |
| 45 | TTTCAATTCTAGATCAGGA | 46 | TCCTGATCTAGAATTGAAA |
| 47 | TTCAATTCTAGATCAGGAA | 48 | TTCCTGATCTAGAATTGAA |
| 49 | TCAATTCTAGATCAGGAAC | 50 | GTTCCTGATCTAGAATTGA |
| 51 | CAATTCTAGATCAGGAACT | 52 | AGTTCCTGATCTAGAATTG |
| 53 | AATTCTAGATCAGGAACTG | 54 | CAGTTCCTGATCTAGAATT |
| 55 | ATTCTAGATCAGGAACTGA | 56 | TCAGTTCCTGATCTAGAAT |
| 57 | TTCTAGATCAGGAACTGAG | 58 | CTCAGTTCCTGATCTAGAA |
| 59 | TCTAGATCAGGAACTGAGG | 60 | CCTCAGTTCCTGATCTAGA |
| 61 | CTAGATCAGGAACTGAGGA | 62 | TCCTCAGTTCCTGATCTAG |
| 63 | TAGATCAGGAACTGAGGAC | 64 | GTCCTCAGTTCCTGATCTA |
| 65 | AGATCAGGAACTGAGGACA | 66 | TGTCCTCAGTTCCTGATCT |
| 67 | GATCAGGAACTGAGGACAT | 68 | ATGTCCTCAGTTCCTGATC |
| 69 | ATCAGGAACTGAGGACATA | 70 | TATGTCCTCAGTTCCTGAT |
| 71 | TCAGGAACTGAGGACATAT | 72 | ATATGTCCTCAGTTCCTGA |
| 73 | CAGGAACTGAGGACATATC | 74 | GATATGTCCTCAGTTCCTG |
| 75 | AGGAACTGAGGACATATCT | 76 | AGATATGTCCTCAGTTCCT |
| 77 | GGAACTGAGGACATATCTA | 78 | TAGATATGTCCTCAGTTCC |
| 79 | GAACTGAGGACATATCTAA | 80 | TTAGATATGTCCTCAGTTC |
| 81 | AACTGAGGACATATCTAAA | 82 | TTTAGATATGTCCTCAGTT |
| 83 | ACTGAGGACATATCTAAAT | 84 | ATTTAGATATGTCCTCAGT |
| 85 | CTGAGGACATATCTAAATT | 86 | AATTTAGATATGTCCTCAG |
| 87 | TGAGGACATATCTAAATTT | 88 | AAATTTAGATATGTCCTCA |
| 89 | GAGGACATATCTAAATTTT | 90 | AAAATTTAGATATGTCCTC |
| 91 | AGGACATATCTAAATTTTC | 92 | GAAAATTTAGATATGTCCT |
| 93 | GGACATATCTAAATTTTCT | 94 | AGAAAATTTAGATATGTCC |
| 95 | GACATATCTAAATTTTCTA | 96 | TAGAAAATTTAGATATGTC |
| 97 | ACATATCTAAATTTTCTAG | 98 | CTAGAAAATTTAGATATGT |
| 99 | CATATCTAAATTTTCTAGT | 100 | ACTAGAAAATTTAGATATG |
| 101 | ATATCTAAATTTTCTAGTT | 102 | AACTAGAAAATTTAGATAT |
| 103 | TATCTAAATTTTCTAGTTT | 104 | AAACTAGAAAATTTAGATA |
| 105 | ATCTAAATTTTCTAGTTTT | 106 | AAAACTAGAAAATTTAGAT |
| 107 | TCTAAATTTTCTAGTTTTA | 108 | TAAAACTAGAAAATTTAGA |
| 109 | CTAAATTTTCTAGTTTTAT | 110 | ATAAAACTAGAAAATTTAG |
| 111 | TAAATTTTCTAGTTTTATA | 112 | TATAAAACTAGAAAATTTA |
| 113 | AAATTTTCTAGTTTTATAG | 114 | CTATAAAACTAGAAAATTT |
| 115 | AATTTTCTAGTTTTATAGA | 116 | TCTATAAAACTAGAAAATT |
| 117 | ATTTTCTAGTTTTATAGAA | 118 | TTCTATAAAACTAGAAAAT |
| 119 | TTTTCTAGTTTTATAGAAG | 120 | CTTCTATAAAACTAGAAAA |
| 121 | TTTCTAGTTTTATAGAAGG | 122 | CCTTCTATAAAACTAGAAA |
| 123 | TTCTAGTTTTATAGAAGGC | 124 | GCCTTCTATAAAACTAGAA |
| 125 | TCTAGTTTTATAGAAGGCT | 126 | AGCCTTCTATAAAACTAGA |
| 127 | CTAGTTTTATAGAAGGCTT | 128 | AAGCCTTCTATAAAACTAG |
| 129 | TAGTTTTATAGAAGGCTTT | 130 | AAAGCCTTCTATAAAACTA |
| 131 | AGTTTTATAGAAGGCTTTT | 132 | AAAAGCCTTCTATAAAACT |
| 133 | GTTTTATAGAAGGCTTTTA | 134 | TAAAAGCCTTCTATAAAAC |
| 135 | TTTTATAGAAGGCTTTTAT | 136 | ATAAAAGCCTTCTATAAAA |
| 137 | TTTATAGAAGGCTTTTATC | 138 | GATAAAAGCCTTCTATAAA |
| 139 | TTATAGAAGGCTTTTATCC | 140 | GGATAAAAGCCTTCTATAA |
| 141 | TATAGAAGGCTTTTATCCA | 142 | TGGATAAAAGCCTTCTATA |
| 143 | ATAGAAGGCTTTTATCCAC | 144 | GTGGATAAAAGCCTTCTAT |
| 145 | TAGAAGGCTTTTATCCACA | 146 | TGTGGATAAAAGCCTTCTA |
| 147 | AGAAGGCTTTTATCCACAA | 148 | TTGTGGATAAAAGCCTTCT |
| 149 | GAAGGCTTTTATCCACAAG | 150 | CTTGTGGATAAAAGCCTTC |
| 151 | AAGGCTTTTATCCACAAGA | 152 | TCTTGTGGATAAAAGCCTT |
| 153 | AGGCTTTTATCCACAAGAA | 154 | TTCTTGTGGATAAAAGCCT |
| 155 | GGCTTTTATCCACAAGAAT | 156 | ATTCTTGTGGATAAAAGCC |
| 157 | GCTTTTATCCACAAGAATC | 158 | GATTCTTGTGGATAAAAGC |
| 159 | CTTTTATCCACAAGAATCA | 160 | TGATTCTTGTGGATAAAAG |
| 161 | TTTTATCCACAAGAATCAA | 162 | TTGATTCTIGTGGATAAAA |
| 163 | TTTATCCACAAGAATCAAG | 164 | CTTGATTCTTGTGGATAAA |
| 165 | TTATCCACAAGAATCAAGA | 166 | TCTTGATTCTTGTGGATAA |
| 167 | TATCCACAAGAATCAAGAT | 168 | ATCTTGATTCTTGTGGATA |
| 169 | ATCCACAAGAATCAAGATC | 170 | GATCTTGATTCTTGTGGAT |
| 171 | TCCACAAGAATCAAGATCT | 172 | AGATCTTGATTCTTGTGGA |
| 173 | CCACAAGAATCAAGATCTT | 174 | AAGATCTTGATTCTTGTGG |
| 175 | CACAAGAATCAAGATCTTC | 176 | GAAGATCTTGATTCTTGTG |
| 177 | ACAAGAATCAAGATCTTCC | 178 | GGAAGATCTTGATTCTTGT |
| 179 | CAAGAATCAAGATCTTCCC | 180 | GGGAAGATCTTGATTCTTG |
| 181 | AAGAATCAAGATCTTCCCT | 182 | AGGGAAGATCTTGATTCTT |
| 183 | AGAATCAAGATCTTCCCTC | 184 | GAGGGAAGATCTTGATTCT |
| 185 | GAATCAAGATCTTCCCTCT | 186 | AGAGGGAAGATCTTGATTC |
| 187 | AATCAAGATCTTCCCTCTC | 188 | GAGAGGGAAGATCTTGATT |
| 189 | ATCAAGATCTTCCCTCTCT | 190 | AGAGAGGGAAGATCTTGAT |
| 191 | TCAAGATCTTCCCTCTCTG | 192 | CAGAGAGGGAAGATCTTGA |
| 193 | CAAGATCTTCCCTCTCTGA | 194 | TCAGAGAGGGAAGATCTTG |
| 195 | AAGATCTTCCCTCTCTGAG | 196 | CTCAGAGAGGGAAGATCTT |
| 197 | AGATCTTCCCTCTCTGAGC | 198 | GCTCAGAGAGGGAAGATCT |
| 199 | GATCTTCCCTCTCTGAGCA | 200 | TGCTCAGAGAGGGAAGATC |
| 201 | ATCTTCCCTCTCTGAGCAG | 202 | CTGCTCAGAGAGGGAAGAT |
| 203 | TCTTCCCTCTCTGAGCAGG | 204 | CCTGCTCAGAGAGGGAAGA |
| 205 | CTTCCCTCTCTGAGCAGGA | 206 | TCCTGCTCAGAGAGGGAAG |
| 207 | TTCCCTCTCTGAGCAGGAA | 208 | TTCCTGCTCAGAGAGGGAA |
| 209 | TCCCTCTCTGAGCAGGAAT | 210 | ATTCCTGCTCAGAGAGGGA |
| 211 | CCCTCTCTGAGCAGGAATC | 212 | GATTCCTGCTCAGAGAGGG |
| 213 | CCTCTCTGAGCAGGAATCC | 214 | GGATTCCTGCTCAGAGAGG |
| 215 | CTCTCTGAGCAGGAATCCT | 216 | AGGATTCCTGCTCAGAGAG |
| 217 | TCTCTGAGCAGGAATCCTT | 218 | AAGGATTCCTGCTCAGAGA |
| 219 | CTCTGAGCAGGAATCCTTT | 220 | AAAGGATTCCTGCTCAGAG |
| 221 | TCTGAGCAGGAATCCTTTG | 222 | CAAAGGATTCCTGCTCAGA |
| 223 | CTGAGCAGGAATCCTTTGT | 224 | ACAAAGGATTCCTGCTCAG |
| 225 | TGAGCAGGAATCCTTTGTG | 226 | CACAAAGGATTCCTGCTCA |
| 227 | GAGCAGGAATCCTTTGTGC | 228 | GCACAAAGGATTCCTGCTC |
| 229 | AGCAGGAATCCTTTGTGCA | 230 | TGCACAAAGGATTCCTGCT |
| 231 | GCAGGAATCCTTTGTGCAT | 232 | ATGCACAAAGGATTCCTGC |
| 233 | CAGGAATCCTTTGTGCATT | 234 | AATGCACAAAGGATTCCTG |
| 235 | AGGAATCCTTTGTGCATTG | 236 | CAATGCACAAAGGATTCCT |
| 237 | GGAATCCTTTGTGCATTGA | 238 | TCAATGCACAAAGGATTCC |
| 239 | GAATCCTTTGTGCATTGAA | 240 | TTCAATGCACAAAGGATTC |
| 241 | AATCCTTTGTGCATTGAAG | 242 | CTTCAATGCACAAAGGATT |
| 243 | ATCCTTTGTGCATTGAAGA | 244 | TCTTCAATGCACAAAGGAT |
| 245 | TCCTTTGTGCATTGAAGAC | 246 | GTCTTCAATGCACAAAGGA |
| 247 | CCTTTGTGCATTGAAGACT | 248 | AGTCTTCAATGCACAAAGG |
| 249 | CTTTGTGCATTGAAGACTT | 250 | AAGTCTTCAATGCACAAAG |
| 251 | TTTGTGCATTGAAGACTTT | 252 | AAAGTCTTCAATGCACAAA |
| 253 | TTGTGCATTGAAGACTTTA | 254 | TAAAGTCTTCAATGCACAA |
| 255 | TGTGCATTGAAGACTTTAG | 256 | CTAAAGTCTTCAATGCACA |
| 257 | GTGCATTGAAGACTTTAGA | 258 | TCTAAAGTCTTCAATGCAC |
| 259 | TGCATTGAAGACTTTAGAT | 260 | ATCTAAAGTCTTCAATGCA |
| 261 | GCATTGAAGACTTTAGATT | 262 | AATCTAAAGTCTTCAATGC |
| 263 | CATTGAAGACTTTAGATTC | 264 | GAATCTAAAGTCTTCAATG |
| 265 | ATTGAAGACTTTAGATTCC | 266 | GGAATCTAAAGTCTTCAAT |
| 267 | TTGAAGACTTTAGATTCCT | 268 | AGGAATCTAAAGTCTTCAA |
| 269 | TGAAGACTTTAGATTCCTC | 270 | GAGGAATCTAAAGTCTTCA |
| 271 | GAAGACTTTAGATTCCTCT | 272 | AGAGGAATCTAAAGTCTTC |
| 273 | AAGACTTTAGATTCCTCTC | 274 | GAGAGGAATCTAAAGTCTT |
| 275 | AGACTTTAGATTCCTCTCT | 276 | AGAGAGGAATCTAAAGTCT |
| 277 | GACTTTAGATTCCTCTCTG | 278 | CAGAGAGGAATCTAAAGTC |
| 279 | ACTTTAGATTCCTCTCTGC | 280 | GCAGAGAGGAATCTAAAGT |
| 281 | CTTTAGATTCCTCTCTGCG | 282 | CGCAGAGAGGAATCTAAAG |
| 283 | TTTAGATTCCTCTCTGCGG | 284 | CCGCAGAGAGGAATCTAAA |
| 285 | TTAGATTCCTCTCTGCGGT | 286 | ACCGCAGAGAGGAATCTAA |
| 287 | TAGATTCCTCTCTGCGGTA | 288 | TACCGCAGAGAGGAATCTA |
| 289 | AGATTCCTCTCTGCGGTAG | 290 | CTACCGCAGAGAGGAATCT |
| 291 | GATTCCTCTCTGCGGTAGA | 292 | TCTACCGCAGAGAGGAATC |
| 293 | ATTCCTCTCTGCGGTAGAC | 294 | GTCTACCGCAGAGAGGAAT |
| 295 | TTCCTCTCTGCGGTAGACG | 296 | CGTCTACCGCAGAGAGGAA |
| 297 | TCCTCTCTGCGGTAGACGT | 298 | ACGTCTACCGCAGAGAGGA |
| 299 | CCTCTCTGCGGTAGACGTG | 300 | CACGTCTACCGCAGAGAGG |
| 301 | CTCTCTGCGGTAGACGTGC | 302 | GCACGTCTACCGCAGAGAG |
| 303 | TCTCTGCGGTAGACGTGCA | 304 | TGCACGTCTACCGCAGAGA |
| 305 | CTCTGCGGTAGACGTGCAC | 306 | GTGCACGTCTACCGCAGAG |
| 307 | TCTGCGGTAGACGTGCACT | 308 | AGTGCACGTCTACCGCAGA |
| 309 | CTGCGGTAGACGTGCACTT | 310 | AAGTGCACGTCTACCGCAG |
| 311 | TGCGGTAGACGTGCACTTA | 312 | TAAGTGCACGTCTACCGCA |
| 313 | GCGGTAGACGTGCACTTAT | 314 | ATAAGTGCACGTCTACCGC |
| 315 | CGGTAGACGTGCACTTATA | 316 | TATAAGTGCACGTCTACCG |
| 317 | GGTAGACGTGCACTTATAA | 318 | TTATAAGTGCACGTCTACC |
| 319 | GTAGACGTGCACTTATAAG | 320 | CTTATAAGTGCACGTCTAC |
| 321 | TAGACGTGCACTTATAAGT | 322 | ACTTATAAGTGCACGTCTA |
| 323 | AGACGTGCACTTATAAGTA | 324 | TACTTATAAGTGCACGTCT |
| 325 | GACGTGCACTTATAAGTAT | 326 | ATACTTATAAGTGCACGTC |
| 327 | ACGTGCACTTATAAGTATT | 328 | AATACTTATAAGTGCACGT |
| 329 | CGTGCACTTATAAGTATTT | 330 | AAATACTTATAAGTGCACG |
| 331 | GTGCACTTATAAGTATTTG | 332 | CAAATACTTATAAGTGCAC |
| 333 | TGCACTTATAAGTATTTGA | 334 | TCAAATACTTATAAGTGCA |
| 335 | GCACTTATAAGTATTTGAT | 336 | ATCAAATACTTATAAGTGC |
| 337 | CACTTATAAGTATTTGATG | 338 | CATCAAATACTTATAAGTG |
| 339 | ACTTATAAGTATTTGATGG | 340 | CCATCAAATACTTATAAGT |
| 341 | CTTATAAGTATTTGATGGG | 342 | CCCATCAAATACTTATAAG |
| 343 | TTATAAGTATTTGATGGGG | 344 | CCCCATCAAATACTTATAA |
| 345 | TATAAGTATTTGATGGGGT | 346 | ACCCCATCAAATACTTATA |
| 347 | ATAAGTATTTGATGGGGTG | 348 | CACCCCATCAAATACTTAT |
| 349 | TAAGTATTTGATGGGGTGG | 350 | CCACCCCATCAAATACTTA |
| 351 | AAGTATTTGATGGGGTGGA | 352 | TCCACCCCATCAAATACTT |
| 353 | AGTATTTGATGGGGTGGAT | 354 | ATCCACCCCATCAAATACT |
| 355 | GTATTTGATGGGGTGGATT | 356 | AATCCACCCCATCAAATAC |
| 357 | TATTTGATGGGGTGGATTC | 358 | GAATCCACCCCATCAAATA |
| 359 | ATTTGATGGGGTGGATTCG | 360 | CGAATCCACCCCATCAAAT |
| 361 | TTTGATGGGGTGGATTCGT | 362 | ACGAATCCACCCCATCAAA |
| 363 | TTGATGGGGTGGATTCGTG | 364 | CACGAATCCACCCCATCAA |
| 365 | TGATGGGGTGGATTCGTGG | 366 | CCACGAATCCACCCCATCA |
| 367 | GATGGGGTGGATTCGTGGT | 368 | ACCACGAATCCACCCCATC |
| 369 | ATGGGGTGGATTCGTGGTC | 370 | GACCACGAATCCACCCCAT |
| 371 | TGGGGTGGATTCGTGGTCG | 372 | CGACCACGAATCCACCCCA |
| 373 | GGGGTGGATTCGTGGTCGG | 374 | CCGACCACGAATCCACCCC |
| 375 | GGGTGGATTCGTGGTCGGA | 376 | TCCGACCACGAATCCACCC |
| 377 | GGTGGATTCGTGGTCGGAG | 378 | CTCCGACCACGAATCCACC |
| 379 | GTGGATTCGTGGTCGGAGG | 380 | CCTCCGACCACGAATCCAC |
| 381 | TGGATTCGTGGTCGGAGGT | 382 | ACCTCCGACCACGAATCCA |
| 383 | GGATTCGTGGTCGGAGGTC | 384 | GACCTCCGACCACGAATCC |
| 385 | GATTCGTGGTCGGAGGTCT | 386 | AGACCTCCGACCACGAATC |
| 387 | ATTCGTGGTCGGAGGTCTC | 388 | GAGACCTCCGACCACGAAT |
| 389 | TTCGTGGTCGGAGGTCTCG | 390 | CGAGACCTCCGACCACGAA |
| 391 | TCGTGGTCGGAGGTCTCGA | 392 | TCGAGACCTCCGACCACGA |
| 393 | CGTGGTCGGAGGTCTCGAC | 394 | GTCGAGACCTCCGACCACG |
| 395 | GTGGTCGGAGGTCTCGACA | 396 | TGTCGAGACCTCCGACCAC |
| 397 | TGGTCGGAGGTCTCGACAC | 398 | GTGTCGAGACCTCCGACCA |
| 399 | GGTCGGAGGTCTCGACACA | 400 | TGTGTCGAGACCTCCGACC |
| 401 | GTCGGAGGTCTCGACACAG | 402 | CTGTGTCGAGACCTCCGAC |
| 403 | TCGGAGGTCTCGACACAGC | 404 | GCTGTGTCGAGACCTCCGA |
| 405 | CGGAGGTCTCGACACAGCT | 406 | AGCTGTGTCGAGACCTCCG |
| 407 | GGAGGTCTCGACACAGCTG | 408 | CAGCTGTGTCGAGACCTCC |
| 409 | GAGGTCTCGACACAGCTGG | 410 | CCAGCTGTGTCGAGACCTC |
| 411 | AGGTCTCGACACAGCTGGG | 412 | CCCAGCTGTGTCGAGACCT |
| 413 | GGTCTCGACACAGCTGGGA | 414 | TCCCAGCTGTGTCGAGACC |
| 415 | GTCTCGACACAGCTGGGAG | 416 | CTCCCAGCTGTGTCGAGAC |
| 417 | TCTCGACACAGCTGGGAGA | 418 | TCTCCCAGCTGTGTCGAGA |
| 419 | CTCGACACAGCTGGGAGAT | 420 | ATCTCCCAGCTGTGTCGAG |
| 421 | TCGACACAGCTGGGAGATG | 422 | CATCTCCCAGCTGTGTCGA |
| 423 | CGACACAGCTGGGAGATGA | 424 | TCATCTCCCAGCTGTGTCG |
| 425 | GACACAGCTGGGAGATGAG | 426 | CTCATCTCCCAGCTGTGTC |
| 427 | ACACAGCTGGGAGATGAGT | 428 | ACTCATCTCCCAGCTGTGT |
| 429 | CACAGCTGGGAGATGAGTG | 430 | CACTCATCTCCCAGCTGTG |
| 431 | ACAGCTGGGAGATGAGTGA | 432 | TCACTCATCTCCCAGCTGT |
| 433 | CAGCTGGGAGATGAGTGAA | 434 | TTCACTCATCTCCCAGCTG |
| 435 | AGCTGGGAGATGAGTGAAT | 436 | ATTCACTCATCTCCCAGCT |
| 437 | GCTGGGAGATGAGTGAATT | 438 | AATTCACTCATCTCCCAGC |
| 439 | CTGGGAGATGAGTGAATTT | 440 | AAATTCACTCATCTCCCAG |
| 441 | TGGGAGATGAGTGAATTTC | 442 | GAAATTCACTCATCTCCCA |
| 443 | GGGAGATGAGTGAATTTCA | 444 | TGAAATTCACTCATCTCCC |
| 445 | GGAGATGAGTGAATTTCAT | 446 | ATGAAATTCACTCATCTCC |
| 447 | GAGATGAGTGAATTTCATA | 448 | TATGAAATTCACTCATCTC |
| 449 | AGATGAGTGAATTTCATAA | 450 | TTATGAAATTCACTCATCT |
| 451 | GATGAGTGAATTTCATAAT | 452 | ATTATGAAATTCACTCATC |
| 453 | ATGAGTGAATTTCATAATT | 454 | AATTATGAAATTCACTCAT |
| 455 | TGAGTGAATTTCATAATTA | 456 | TAATTATGAAATTCACTCA |
| 457 | GAGTGAATTTCATAATTAT | 458 | ATAATTATGAAATTCACTC |
| 459 | AGTGAATTTCATAATTATA | 460 | TATAATTATGAAATTCACT |
| 461 | GTGAATTTCATAATTATAA | 462 | TTATAATTATGAAATTCAC |
| 463 | TGAATTTCATAATTATAAC | 464 | GTTATAATTATGAAATTCA |
| 465 | GAATTTCATAATTATAACT | 466. | AGTTATAATTATGAAATTC |
| 467 | AATTTCATAATTATAACTT | 468 | AAGTTATAATTATGAAATT |
| 469 | ATTTCATAATTATAACTTG | 470 | CAAGTTATAATTATGAAAT |
| 471 | TTTCATAATTATAACTTGG | 472 | CCAAGTTATAATTATGAAA |
| 473 | TTCATAATTATAACTTGGA | 474 | TCCAAGTTATAATTATGAA |
| 475 | TCATAATTATAACTTGGAT | 476 | ATCCAAGTTATAATTATGA |
| 477 | CATAATTATAACTTGGATC | 478 | GATCCAAGTTATAATTATG |
| 479 | ATAATTATAACTTGGATCT | 480 | AGATCCAAGTTATAATTAT |
| 481 | TAATTATAACTTGGATCTG | 482 | CAGATCCAAGTTATAATTA |
| 483 | AATTATAACTTGGATCTGA | 484 | TCAGATCCAAGTTATAATT |
| 485 | ATTATAACTTGGATCTGAA | 486 | TTCAGATCCAAGTTATAAT |
| 487 | TTATAACTTGGATCTGAAG | 488 | CTTCAGATCCAAGTTATAA |
| 489 | TATAACTTGGATCTGAAGA | 490 | TCTTCAGATCCAAGTTATA |
| 491 | ATAACTTGGATCTGAAGAA | 492 | TTCTTCAGATCCAAGTTAT |
| 493 | TAACTTGGATCTGAAGAAG | 494 | CTTCTTCAGATCCAAGTTA |
| 495 | AACTTGGATCTGAAGAAGA | 496 | TCTTCTTCAGATCCAAGTT |
| 497 | ACTTGGATCTGAAGAAGAG | 498 | CTCTTCTTCAGATCCAAGT |
| 499 | CTTGGATCTGAAGAAGAGT | 500 | ACTCTTCTTCAGATCCAAG |
| 501 | TTGGATCTGAAGAAGAGTG | 502 | CACTCTTCTTCAGATCCAA |
| 503 | TGGATCTGAAGAAGAGTGA | 504 | TCACTCTTCTTCAGATCCA |
| 505 | GGATCTGAAGAAGAGTGAT | 506 | ATCACTCTTCTTCAGATCC |
| 507 | GATCTGAAGAAGAGTGATT | 508 | AATCACTCTTCTTCAGATC |
| 509 | ATCTGAAGAAGAGTGATTT | 510 | AAATCACTCTTCTTCAGAT |
| 511 | TCTGAAGAAGAGTGATTTT | 512 | AAAATCACTCTTCTTCAGA |
| 513 | CTGAAGAAGAGTGATTTTT | 514 | AAAAATCACTCTTCTTCAG |
| 515 | TGAAGAAGAGTGATTTTTC | 516 | GAAAAATCACTCTTCTTCA |
| 517 | GAAGAAGAGTGATTTTTCA | 518 | TGAAAAATCACTCTTCTTC |
| 519 | AAGAAGAGTGATTTTTCAA | 520 | TTGAAAAATCACTCTTCTT |
| 521 | AGAAGAGTGATTTTTCAAC | 522 | GTTGAAAAATCACTCTTCT |
| 523 | GAAGAGTGATTTTTCAACA | 524 | TGTTGAAAAATCACTCTTC |
| 525 | AAGAGTGATTTTTCAACAC | 526 | GTGTTGAAAAATCACTCTT |
| 527 | AGAGTGATTTTTCAACACG | 528 | CGTGTTGAAAAATCACTCT |
| 529 | GAGTGATTTTTCAACACGA | 530 | TCGTGTTGAAAAATCACTC |
| 531 | AGTGATTTTTCAACACGAT | 532 | ATCGTGTTGAAAAATCACT |
| 533 | GTGATTTTTCAACACGATG | 534 | CATCGTGTTGAAAAATCAC |
| 535 | TGATTTTTCAACACGATGG | 536 | CCATCGTGTTGAAAAATCA |
| 537 | GATTTTTCAACACGATGGC | 538 | GCCATCGTGTTGAAAAATC |
| 539 | ATTTTTCAACACGATGGCA | 540 | TGCCATCGTGTTGAAAAAT |
| 541 | TTTTTCAACACGATGGCAA | 542 | TTGCCATCGTGTTGAAAAA |
| 543 | TTTTCAACACGATGGCAAA | 544 | TTTGCCATCGTGTTGAAAA |
| 545 | TTTCAACACGATGGCAAAA | 546 | TTTTGCCATCGTGTTGAAA |
| 547 | TTCAACACGATGGCAAAAG | 548 | CTTTTGCCATCGTGTTGAA |
| 549 | TCAACACGATGGCAAAAGC | 550 | GCTTTTGCCATCGTGTTGA |
| 551 | CAACACGATGGCAAAAGCA | 552 | TGCTTTTGCCATCGTGTTG |
| 553 | AACACGATGGCAAAAGCAA | 554 | TTGCTTTTGCCATCGTGTT |
| 555 | ACACGATGGCAAAAGCAAA | 556 | TTTGCTTTTGCCATCGTGT |
| 557 | CACGATGGCAAAAGCAAAG | 558 | CTTTGCTTTTGCCATCGTG |
| 559 | ACGATGGCAAAAGCAAAGA | 560 | TCTTTGCTTTTGCCATCGT |
| 561 | CGATGGCAAAAGCAAAGAT | 562 | ATCTTTGCTTTTGCCATCG |
| 563 | GATGGCAAAAGCAAAGATG | 564 | CATCTTTGCTTTTGCCATC |
| 565 | ATGGCAAAAGCAAAGATGT | 566 | ACATCTTTGCTTTTGCCAT |
| 567 | TGGCAAAAGCAAAGATGTC | 568 | GACATCTTTGCTTTTGCCA |
| 569 | GGCAAAAGCAAAGATGTCC | 570 | GGACATCTTTGCTTTTGCC |
| 571 | GCAAAAGCAAAGATGTCCA | 572 | TGGACATCTTTGCTTTTGC |
| 573 | CAAAAGCAAAGATGTCCAG | 574 | CTGGACATCTTTGCTTTTG |
| 575 | AAAAGCAAAGATGTCCAGT | 576 | ACTGGACATCTTTGCTTTT |
| 577 | AAAGCAAAGATGTCCAGTA | 578 | TACTGGACATCTTTGCTTT |
| 579 | AAGCAAAGATGTCCAGTAG | 580 | CTACTGGACATCTTTGCTT |
| 581 | AGCAAAGATGTCCAGTAGT | 582 | ACTACTGGACATCTTTGCT |
| 583 | GCAAAGATGTCCAGTAGTC | 584 | GACTACTGGACATCTTTGC |
| 585 | CAAAGATGTCCAGTAGTCA | 586 | TGACTACTGGACATCTTTG |
| 587 | AAAGATGTCCAGTAGTCAA | 588 | TTGACTACTGGACATCTTT |
| 589 | AAGATGTCCAGTAGTCAAA | 590 | TTTGACTACTGGACATCTT |
| 591 | AGATGTCCAGTAGTCAAAA | 592 | TTTTGACTACTGGACATCT |
| 593 | GATGTCCAGTAGTCAAAAG | 594 | CTTTTGACTACTGGACATC |
| 595 | ATGTCCAGTAGTCAAAAGC | 596 | GCTTTTGACTACTGGACAT |
| 597 | TGTCCAGTAGTCAAAAGCA | 598 | TGCTTTTGACTACTGGACA |
| 599 | GTCCAGTAGTCAAAAGCAA | 600 | TTGCTTTTGACTACTGGAC |
| 601 | TCCAGTAGTCAAAAGCAAA | 602 | TTTGCTTTTGACTACTGGA |
| 603 | CCAGTAGTCAAAAGCAAAT | 604 | ATTTGCTTTTGACTACTGG |
| 605 | CAGTAGTCAAAAGCAAATG | 606 | CATTTGCTTTTGACTACTG |
| 607 | AGTAGTCAAAAGCAAATGT | 608 | ACATTTGCTTTTGACTACT |
| 609 | GTAGTCAAAAGCAAATGTA | 610 | TACATTTGCTTTTGACTAC |
| 611 | TAGTCAAAAGCAAATGTAG | 612 | CTACATTTGCTTTTGACTA |
| 613 | AGTCAAAAGCAAATGTAGA | 614 | TCTACATTTGCTTTTGACT |
| 615 | GTCAAAAGCAAATGTAGAG | 616 | CTCTACATTTGCTTTTGAC |
| 617 | TCAAAAGCAAATGTAGAGA | 618 | TCTCTACATTTGCTTTTGA |
| 619 | CAAAAGCAAATGTAGAGAA | 620 | TTCTCTACATTTGCTTTTG |
| 621 | AAAAGCAAATGTAGAGAAA | 622 | TTTCTCTACATTTGCTTTT |
| 623 | AAAGCAAATGTAGAGAAAA | 624 | TTTTCTCTACATTTGCTTT |
| 625 | AAGCAAATGTAGAGAAAAT | 626 | ATTTTCTCTACATTTGCTT |
| 627 | AGCAAATGTAGAGAAAATG | 628 | CATTTTCTCTACATTTGCT |
| 629 | GCAAATGTAGAGAAAATGC | 630 | GCATTTTCTCTACATTTGC |
| 631 | CAAATGTAGAGAAAATGCA | 632 | TGCATTTTCTCTACATTTG |
| 633 | AAATGTAGAGAAAATGCAT | 634 | ATGCATTTTCTCTACATTT |
| 635 | AATGTAGAGAAAATGCATC | 636 | GATGCATTTTCTCTACATT |
| 637 | ATGTAGAGAAAATGCATCT | 638 | AGATGCATTTTCTCTACAT |
| 639 | TGTAGAGAAAATGCATCTC | 640 | GAGATGCATTTTCTCTACA |
| 641 | GTAGAGAAAATGCATCTCC | 642 | GGAGATGCATTTTCTCTAC |
| 643 | TAGAGAAAATGCATCTCCA | 644 | TGGAGATGCATTTTCTCTA |
| 645 | AGAGAAAATGCATCTCCAT | 646 | ATGGAGATGCATTTTCTCT |
| 647 | GAGAAAATGCATCTCCATT | 648 | AATGGAGATGCATTTTCTC |
| 649 | AGAAAATGCATCTCCATTT | 650 | AAATGGAGATGCATTTTCT |
| 651 | GAAAATGCATCTCCATTTT | 652 | AAAATGGAGATGCATTTTC |
| 653 | AAAATGCATCTCCATTTTT | 654 | AAAAATGGAGATGCATTTT |
| 655 | AAATGCATCTCCATTTTTT | 656 | AAAAAATGGAGATGCATTT |
| 657 | AATGCATCTCCATTTTTTT | 658 | AAAAAAATGGAGATGCATT |
| 659 | ATGCATCTCCATTTTTTTT | 660 | AAAAAAAATGGAGATGCAT |
| 661 | TGCATCTCCATTTTTTTTC | 662 | GAAAAAAAATGGAGATGCA |
| 663. | GCATCTCCATTTTTTTTCT | 664 | AGAAAAAAAATGGAGATGC |
| 665 | CATCTCCATTTTTTTTCTG | 666 | CAGAAAAAAAATGGAGATG |
| 667 | ATCTCCATTTTTTTTCTGC | 668 | GCAGAAAAAAAATGGAGAT |
| 669 | TCTCCATTTTTTTTCTGCT | 670 | AGCAGAAAAAAAATGGAGA |
| 671 | CTCCATTTTTTTTCTGCTG | 672 | CAGCAGAAAAAAAATGGAG |
| 673 | TCCATTTTTTTTCTGCTGC | 674 | GCAGCAGAAAAAAAATGGA |
| 675 | CCATTTTTTTTCTGCTGCT | 676 | AGCAGCAGAAAAAAAATGG |
| 677 | CATTTTTTTTCTGCTGCTT | 678 | AAGCAGCAGAAAAAAAATG |
| 679 | ATTTTTTTTCTGCTGCTTC | 680 | GAAGCAGCAGAAAAAAAAT |
| 681 | TTTTTTTTCTGCTGCTTCA | 682 | TGAAGCAGCAGAAAAAAAA |
| 683 | TTTTTTTCTGCTGCTTCAT | 684 | ATGAAGCAGCAGAAAAAAA |
| 685 | TTTTTTCTGCTGCTTCATC | 686 | GATGAAGCAGCAGAAAAAA |
| 687 | TTTTTCTGCTGCTTCATCG | 688 | CGATGAAGCAGCAGAAAAA |
| 689 | TTTTCTGCTGCTTCATCGC | 690 | GCGATGAAGCAGCAGAAAA |
| 691 | TTTCTGCTGCTTCATCGCT | 692 | AGCGATGAAGCAGCAGAAA |
| 693 | TTCTGCTGCTTCATCGCTG | 694 | CAGCGATGAAGCAGCAGAA |
| 695 | TCTGCTGCTTCATCGCTGT | 696 | ACAGCGATGAAGCAGCAGA |
| 697 | CTGCTGCTTCATCGCTGTA | 698 | TACAGCGATGAAGCAGCAG |
| 699 | TGCTGCTTCATCGCTGTAG | 700 | CTACAGCGATGAAGCAGCA |
| 701 | GCTGCTTCATCGCTGTAGC | 702 | GCTACAGCGATGAAGCAGC |
| 703 | CTGCTTCATCGCTGTAGCC | 704 | GGCTACAGCGATGAAGCAG |
| 705 | TGCTTCATCGCTGTAGCCA | 706 | TGGCTACAGCGATGAAGCA |
| 707 | GCTTCATCGCTGTAGCCAT | 708 | ATGGCTACAGCGATGAAGC |
| 709 | CTTCATCGCTGTAGCCATG | 710 | CATGGCTACAGCGATGAAG |
| 711 | TTCATCGCTGTAGCCATGG | 712 | CCATGGCTACAGCGATGAA |
| 713 | TCATCGCTGTAGCCATGGG | 714 | CCCATGGCTACAGCGATGA |
| 715 | CATCGCTGTAGCCATGGGA | 716 | TCCCATGGCTACAGCGATG |
| 717 | ATCGCTGTAGCCATGGGAA | 718 | TTCCCATGGCTACAGCGAT |
| 719 | TCGCTGTAGCCATGGGAAT | 720 | ATTCCCATGGCTACAGCGA |
| 721 | CGCTGTAGCCATGGGAATC | 722 | GATTCCCATGGCTACAGCG |
| 723 | GCTGTAGCCATGGGAATCC | 724 | GGATTCCCATGGCTACAGC |
| 725 | CTGTAGCCATGGGAATCCG | 726 | CGGATTCCCATGGCTACAG |
| 727 | TGTAGCCATGGGAATCCGT | 728 | ACGGATTCCCATGGCTACA |
| 729 | GTAGCCATGGGAATCCGTT | 730 | AACGGATTCCCATGGCTAC |
| 731 | TAGCCATGGGAATCCGTTT | 732 | AAACGGATTCCCATGGCTA |
| 733 | AGCCATGGGAATCCGTTTC | 734 | GAAACGGATTCCCATGGCT |
| 735 | GCCATGGGAATCCGTTTCA | 736 | TGAAACGGATTCCCATGGC |
| 737 | CCATGGGAATCCGTTTCAT | 738 | ATGAAACGGATTCCCATGG |
| 739 | CATGGGAATCCGTTTCATT | 740 | AATGAAACGGATTCCCATG |
| 741 | ATGGGAATCCGTTTCATTA | 742 | TAATGAAACGGATTCCCAT |
| 743 | TGGGAATCCGTTTCATTAT | 744 | ATAATGAAACGGATTCCCA |
| 745 | GGGAATCCGTTTCATTATT | 746 | AATAATGAAACGGATTCCC |
| 747 | GGAATCCGTTTCATTATTA | 748 | TAATAATGAAACGGATTCC |
| 749 | GAATCCGTTTCATTATTAT | 750 | ATAATAATGAAACGGATTC |
| 751 | AATCCGTTTCATTATTATG | 752 | CATAATAATGAAACGGATT |
| 753 | ATCCGTTTCATTATTATGG | 754 | CCATAATAATGAAACGGAT |
| 755 | TCCGTTTCATTATTATGGT | 756 | ACCATAATAATGAAACGGA |
| 757 | CCGTTTCATTATTATGGTA | 758 | TACCATAATAATGAAACGG |
| 759 | CGTTTCATTATTATGGTAA | 760 | TTACCATAATAATGAAACG |
| 761 | GTTTCATTATTATGGTAAC | 762 | GTTACCATAATAATGAAAC |
| 763 | TTTCATTATTATGGTAACA | 764 | TGTTACCATAATAATGAAA |
| 765 | TTCATTATTATGGTAACAA | 766 | TTGTTACCATAATAATGAA |
| 767 | TCATTATTATGGTAACAAT | 768 | ATTGTTACCATAATAATGA |
| 769 | CATTATTATGGTAACAATA | 770 | TATTGTTACCATAATAATG |
| 771 | ATTATTATGGTAACAATAT | 772 | ATATTGTTACCATAATAAT |
| 773 | TTATTATGGTAACAATATG | 774 | CATATTGTTACCATAATAA |
| 775 | TATTATGGTAACAATATGG | 776 | CCATATTGTTACCATAATA |
| 777 | ATTATGGTAACAATATGGA | 778 | TCCATATTGTTACCATAAT |
| 779 | TTATGGTAACAATATGGAG | 780 | CTCCATATTGTTACCATAA |
| 781 | TATGGTAACAATATGGAGT | 782 | ACTCCATATTGTTACCATA |
| 783 | ATGGTAACAATATGGAGTG | 784 | CACTCCATATTGTTACCAT |
| 785 | TGGTAACAATATGGAGTGC | 786 | GCACTCCATATTGTTACCA |
| 787 | GGTAACAATATGGAGTGCT | 788 | AGCACTCCATATTGTTACC |
| 789 | GTAACAATATGGAGTGCTG | 790 | CAGCACTCCATATTGTTAC |
| 791 | TAACAATATGGAGTGCTGT | 792 | ACAGCACTCCATATTGTTA |
| 793 | AACAATATGGAGTGCTGTA | 794 | TACAGCACTCCATATTGTT |
| 795 | ACAATATGGAGTGCTGTAT | 796 | ATACAGCACTCCATATTGT |
| 797 | CAATATGGAGTGCTGTATT | 798 | AATACAGCACTCCATATTG |
| 799 | AATATGGAGTGCTGTATTC | 800 | GAATACAGCACTCCATATT |
| 801 | ATATGGAGTGCTGTATTCC | 802 | GGAATACAGCACTCCATAT |
| 803 | TATGGAGTGCTGTATTCCT | 804 | AGGAATACAGCACTCCATA |
| 805 | ATGGAGTGCTGTATTCCTA | 806 | TAGGAATACAGCACTCCAT |
| 807 | TGGAGTGCTGTATTCCTAA | 808 | TTAGGAATACAGCACTCCA |
| 809 | GGAGTGCTGTATTCCTAAA | 810 | TTTAGGAATACAGCACTCC |
| 811 | GAGTGCTGTATTCCTAAAC | 812 | GTTTAGGAATACAGCACTC |
| 813 | AGTGCTGTATTCCTAAACT | 814 | AGTTTAGGAATACAGCACT |
| 815 | GTGCTGTATTCCTAAACTC | 816 | GAGTTTAGGAATACAGCAC |
| 817 | TGCTGTATTCCTAAACTCA | 818 | TGAGTTTAGGAATACAGCA |
| 819 | GCTGTATTCCTAAACTCAT | 820 | ATGAGTTTAGGAATACAGC |
| 821 | CTGTATTCCTAAACTCATT | 822 | AATGAGTTTAGGAATACAG |
| 823 | TGTATTCCTAAACTCATTA | 824 | TAATGAGTTTAGGAATACA |
| 825 | GtATTCCTAAACTCATTAT | 826 | ATAATGAGTTTAGGAATAC |
| 827 | TATTCCTAAACTCATTATT | 828 | AATAATGAGTTTAGGAATA |
| 829 | ATTCCTAAACTCATTATTC | 830 | GAATAATGAGTTTAGGAAT |
| 831 | TTCCTAAACTCATTATTCA | 832 | TGAATAATGAGTTTAGGAA |
| 833 | TCCTAAACTCATTATTCAA | 834 | TTGAATAATGAGTTTAGGA |
| 835 | CCTAAACTCATTATTCAAC | 836 | GTTGAATAATGAGTTTAGG |
| 837 | CTAAACTCATTATTCAACC | 838 | GGTTGAATAATGAGTTTAG |
| 839 | TAAACTCATTATTCAACCA | 840 | TGGTTGAATAATGAGTTTA |
| 841 | AAACTCATTATTCAACCAA | 842 | TTGGTTGAATAATGAGTTT |
| 843 | AACTCATTATTCAACCAAG | 844 | CTTGGTTGAATAATGAGTT |
| 845 | ACTCATTATTCAACCAAGA | 846 | TCTTGGTTGAATAATGAGT |
| 847 | CTCATTATTCAACCAAGAA | 848 | TTCTTGGTTGAATAATGAG |
| 849 | TCATTATTCAACCAAGAAG | 850 | CTTCTTGGTTGAATAATGA |
| 851 | CATTATTCAACCAAGAAGT | 852 | ACTTCTTGGTTGAATAATG |
| 853 | ATTATTCAACCAAGAAGTT | 854 | AACTTCTTGGTTGAATAAT |
| 855 | TTATTCAACCAAGAAGTTC | 856 | GAACTTCTTGGTTGAATAA |
| 857 | TATTCAACCAAGAAGTTCA | 858 | TGAACTTCTTGGTTGAATA |
| 859 | ATTCAACCAAGAAGTTCAA | 860 | TTGAACTTCTTGGTTGAAT |
| 861 | TTCAACCAAGAAGTTCAAA | 862 | TTTGAACTTCTTGGTTGAA |
| 863 | TCAACCAAGAAGTTCAAAT | 864 | ATTTGAACTTCTTGGTTGA |
| 865 | CAACCAAGAAGTTCAAATT | 866 | AATTTGAACTTCTTGGTTG |
| 867 | AACCAAGAAGTTCAAATTC | 868 | GAATTTGAACTTCTTGGTT |
| 869 | ACCAAGAAGTTCAAATTCC | 870 | GGAATTTGAACTTCTTGGT |
| 871 | CCAAGAAGTTCAAATTCCC | 872 | GGGAATTTGAACTTCTTGG |
| 873 | CAAGAAGTTCAAATTCCCT | 874 | AGGGAATTTGAACTTCTTG |
| 875 | AAGAAGTTCAAATTCCCTT | 876 | AAGGGAATTTGAACTTCTT |
| 877 | AGAAGTTCAAATTCCCTTG | 878 | CAAGGGAATTTGAACTTCT |
| 879 | GAAGTTCAAATTCCCTTGA | 880 | TCAAGGGAATTTGAACTTC |
| 881 | AAGTICAAATTCCCTTGAC | 882 | GTCAAGGGAATTTGAACTT |
| 883 | AGTTCAAATTCCCTTGACC | 884 | GGTCAAGGGAATTTGAACT |
| 885 | GTTCAAATTCCCTTGACCG | 886 | CGGTCAAGGGAATTTGAAC |
| 887 | TTCAAATTCCCTTGACCGA | 888 | TCGGTCAAGGGAATTTGAA |
| 889 | TCAAATTCCCTTGACCGAA | 890 | TTCGGTCAAGGGAATTTGA |
| 891 | CAAATTCCCTTGACCGAAA | 892 | TTTCGGTCAAGGGAATTTG |
| 893 | AAATTCCCTTGACCGAAAG | 894 | CTTTCGGTCAAGGGAATTT |
| 895 | AATTCCCTTGACCGAAAGT | 896 | ACTTTCGGTCAAGGGAATT |
| 897 | ATTCCCTTGACCGAAAGTT | 898 | AACTTTCGGTCAAGGGAAT |
| 899 | TTCCCTTGACCGAAAGTTA | 900 | TAACTTTCGGTCAAGGGAA |
| 901 | TCCCTTGACCGAAAGTTAC | 902 | GTAACTTTCGGTCAAGGGA |
| 903 | CCCTTGACCGAAAGTTACT | 904 | AGTAACTTTCGGTCAAGGG |
| 905 | CCTTGACCGAAAGTTACTG | 906 | CAGTAACTTTCGGTCAAGG |
| 907 | CTTGACCGAAAGTTACTGT | 908 | ACAGTAACTTTCGGTCAAG |
| 909 | TTGACCGAAAGTTACTGTG | 910 | CACAGTAACTTTCGGTCAA |
| 911 | TGACCGAAAGTTACTGTGG | 912 | CCACAGTAACTTTCGGTCA |
| 913 | GACCGAAAGTTACTGTGGC | 914 | GCCACAGTAACTTTCGGTC |
| 915 | ACCGAAAGTTACTGTGGCC | 916 | GGCCACAGTAACTTTCGGT |
| 917 | CCGAAAGTTACTGTGGCCC | 918 | GGGCCACAGTAACTTTCGG |
| 919 | CGAAAGTTACTGTGGCCCA | 920 | TGGGCCACAGTAACTTTCG |
| 921 | GAAAGTTACTGTGGCCCAT | 922 | ATGGGCCACAGTAACTTTC |
| 923 | AAAGTTACTGTGGCCCATG | 924 | CATGGGCCACAGTAACTTT |
| 925 | AAGTTACTGTGGCCCATGT | 926 | ACATGGGCCACAGTAACTT |
| 927 | AGTTACTGTGGCCCATGTC | 928 | GACATGGGCCACAGTAACT |
| 929 | GTTACTGTGGCCCATGTCC | 930 | GGACATGGGCCACAGTAAC |
| 931 | TTACTGTGGCCCATGTCCT | 932 | AGGACATGGGCCACAGTAA |
| 933 | TACTGTGGCCCATGTCCTA | 934 | TAGGACATGGGCCACAGTA |
| 935 | ACTGTGGCCCATGTCCTAA | 936 | TTAGGACATGGGCCACAGT |
| 937 | CTGTGGCCCATGTCCTAAA | 938 | TTTAGGACATGGGCCACAG |
| 939 | TGTGGCCCATGTCCTAAAA | 940 | TTTTAGGACATGGGCCACA |
| 941 | GTGGCCCATGTCCTAAAAA | 942 | TTTTTAGGACATGGGCCAC |
| 943 | TGGCCCATGTCCTAAAAAC | 944 | GTTTTTAGGACATGGGCCA |
| 945 | GGCCCATGTCCTAAAAACT | 946 | AGTTTTTAGGACATGGGCC |
| 947 | GCCCATGTCCTAAAAACTG | 948 | CAGTTTTTAGGACATGGGC |
| 949 | CCCATGTCCTAAAAACTGG | 950 | CCAGTTTTTAGGACATGGG |
| 951 | CCATGTCCTAAAAACTGGA | 952 | TCCAGTTTTTAGGACATGG |
| 953 | CATGTCCTAAAAACTGGAT | 954 | ATCCAGTTTTTAGGACATG |
| 955 | ATGTCCTAAAAACTGGATA | 956 | TATCCAGTTTTTAGGACAT |
| 957 | TGTCCTAAAAACTGGATAT | 958 | ATATCCAGTTTTTAGGACA |
| 959 | GTCCTAAAAACTGGATATG | 960 | CATATCCAGTTTTTAGGAC |
| 961 | TCCTAAAAACTGGATATGT | 962 | ACATATCCAGTTTTTAGGA |
| 963 | CCTAAAAACTGGATATGTT | 964 | AACATATCCAGTTTTTAGG |
| 965 | CTAAAAACTGGATATGTTA | 966 | TAACATATCCAGTTTTTAG |
| 967 | TAAAAACTGGATATGTTAC | 968 | GTAACATATCCAGTTTTTA |
| 969 | AAAAACTGGATATGTTACA | 970 | TGTAACATATCCAGTTTTT |
| 971 | AAAACTGGATATGTTACAA | 972 | TTGTAACATATCCAGTTTT |
| 973 | AAACTGGATATGTTACAAA | 974 | TTTGTAACATATCCAGTTT |
| 975 | AACTGGATATGTTACAAAA | 976 | TTTTGTAACATATCCAGTT |
| 977 | ACTGGATATGTTACAAAAA | 978 | TTTTTGTAACATATCCAGT |
| 979 | CTGGATATGTTACAAAAAT | 980 | ATTTTTGTAACATATCCAG |
| 981 | TGGATATGTTACAAAAATA | 982 | TATTTTTGTAACATATCCA |
| 983 | GGATATGTTACAAAAATAA | 984 | TTATTTTTGTAACATATCC |
| 985 | GATATGTTACAAAAATAAC | 986 | GTTATTTTTGTAACATATC |
| 987 | ATATGTTACAAAAATAACT | 988 | AGTTATTTTTGTAACATAT |
| 989 | TATGTTACAAAAATAACTG | 990 | CAGTTATTTTTGTAACATA |
| 991 | ATGTTACAAAAATAACTGC | 992 | GCAGTTATTTTTGTAACAT |
| 993 | TGTTACAAAAATAACTGCT | 994 | AGCAGTTATTTTTGTAACA |
| 995 | GTTACAAAAATAACTGCTA | 996 | TAGCAGTTATTTTTGTAAC |
| 997 | TTACAAAAATAACTGCTAC | 998 | GTAGCAGTTATTTTTGTAA |
| 999 | TACAAAAATAACTGCTACC | 1000 | GGTAGCAGTTATTTTTGTA |
| 1001 | ACAAAAATAACTGCTACCA | 1002 | TGGTAGCAGTTATTTTTGT |
| 1003 | CAAAAATAACTGCTACCAA | 1004 | TTGGTAGCAGTTATTTTTG |
| 1005 | AAAAATAACTGCTACCAAT | 1006 | ATTGGTAGCAGTTATTTTT |
| 1007 | AAAATAACTGCTACCAATT | 1008 | AATTGGTAGCAGTTATTTT |
| 1009 | AAATAACTGCTACCAATTT | 1010 | AAATTGGTAGCAGTTATTT |
| 1011 | AATAACTGCTACCAATTTT | 1012 | AAAATTGGTAGCAGTTATT |
| 1013 | ATAACTGCTACCAATTTTT | 1014 | AAAAATTGGTAGCAGTTAT |
| 1015 | TAACTGCTACCAATTTTTT | 1016 | AAAAAATTGGTAGCAGTTA |
| 1017 | AACTGCTACCAATTTTTTG | 1018 | CAAAAAATTGGTAGCAGTT |
| 1019 | ACTGCTACCAATTTTTTGA | 1020 | TCAAAAAATTGGTAGCAGT |
| 1021 | CTGCTACCAATTTTTTGAT | 1022 | ATCAAAAAATTGGTAGCAG |
| 1023 | TGCTACCAATTTTTTGATG | 1024 | CATCAAAAAATTGGTAGCA |
| 1025 | GCTACCAATTTTTTGATGA | 1026 | TCATCAAAAAATTGGTAGC |
| 1027 | CTACCAATTTTTTGATGAG | 1028 | CTCATCAAAAAATTGGTAG |
| 1029 | TACCAATTTTTTGATGAGA | 1030 | TCTCATCAAAAAATTGGTA |
| 1031 | ACCAATTTTTTGATGAGAG | 1032 | CTCTCATCAAAAAATTGGT |
| 1033 | CCAATTTTTTGATGAGAGT | 1034 | ACTCTCATCAAAAAATTGG |
| 1035 | CAATTTTTTGATGAGAGTA | 1036 | TACTCTCATCAAAAAATTG |
| 1037 | AATTTTTTGATGAGAGTAA | 1038 | TTACTCTCATCAAAAAATT |
| 1039 | ATTTTTTGATGAGAGTAAA | 1040 | TTTACTCTCATCAAAAAAT |
| 1041 | TTTTTTGATGAGAGTAAAA | 1042 | TTTTACTCTCATCAAAAAA |
| 1043 | TTTTTGATGAGAGTAAAAA | 1044 | TTTTTACTCTCATCAAAAA |
| 1045 | TTTTGATGAGAGTAAAAAC | 1046 | GTTTTTACTCTCATCAAAA |
| 1047 | TTTGATGAGAGTAAAAACT | 1048 | AGTTTTTACTCTCATCAAA |
| 1049 | TTGATGAGAGTAAAAACTG | 1050 | CAGTTTTTACTCTCATCAA |
| 1051 | TGATGAGAGTAAAAACTGG | 1052 | CCAGTTTTTACTCTCATCA |
| 1053 | GATGAGAGTAAAAACTGGT | 1054 | ACCAGTTTTTACTCTCATC |
| 1055 | ATGAGAGTAAAAACTGGTA | 1056 | TACCAGTTTTTACTCTCAT |
| 1057 | TGAGAGTAAAAACTGGTAT | 1058 | ATACCAGTTTTTACTCTCA |
| 1059 | GAGAGTAAAAACTGGTATG | 1060 | CATACCAGTTTTTACTCTC |
| 1061 | AGAGTAAAAACTGGTATGA | 1062 | TCATACCAGTTTTTACTCT |
| 1063 | GAGTAAAAACTGGTATGAG | 1064 | CTCATACCAGTTTTTACTC |
| 1065 | AGTAAAAACTGGTATGAGA | 1066 | TCTCATACCAGTTTTTACT |
| 1067 | GTAAAAACTGGTATGAGAG | 1068 | CTCTCATACCAGTTTTTAC |
| 1069 | TAAAAACTGGTATGAGAGC | 1070 | GCTCTCATACCAGTTTTTA |
| 1071 | AAAAACTGGTATGAGAGCC | 1072 | GGCTCTCATACCAGTTTTT |
| 1073 | AAAACTGGTATGAGAGCCA | 1074 | TGGCTCTCATACCAGTTTT |
| 1075 | AAACTGGTATGAGAGCCAG | 1076 | CTGGCTCTCATACCAGTTT |
| 1077 | AACTGGTATGAGAGCCAGG | 1078 | CCTGGCTCTCATACCAGTT |
| 1079 | ACTGGTATGAGAGCCAGGC | 1080 | GCCTGGCTCTCATACCAGT |
| 1081 | CTGGTATGAGAGCCAGGCT | 1082 | AGCCTGGCTCTCATACCAG |
| 1083 | TGGTATGAGAGCCAGGCTT | 1084 | AAGCCTGGCTCTCATACCA |
| 1085 | GGTATGAGAGCCAGGCTTC | 1086 | GAAGCCTGGCTCTCATACC |
| 1087 | GTATGAGAGCCAGGCTTCT | 1088 | AGAAGCCTGGCTCTCATAC |
| 1089 | TATGAGAGCCAGGCTTCTT | 1090 | AAGAAGCCTGGCTCTCATA |
| 1091 | ATGAGAGCCAGGCTTCTTG | 1092 | CAAGAAGCCTGGCTCTCAT |
| 1093 | TGAGAGCCAGGCTTCTTGT | 1094 | ACAAGAAGCCTGGCTCTCA |
| 1095 | GAGAGCCAGGCTTCTTGTA | 1096 | TACAAGAAGCCTGGCTCTC |
| 1097 | AGAGCCAGGCTTCTTGTAT | 1098 | ATACAAGAAGCCTGGCTCT |
| 1099 | GAGCCAGGCTTCTTGTATG | 1100 | CATACAAGAAGCCTGGCTC |
| 1101 | AGCCAGGCTTCTTGTATGT | 1102 | ACATACAAGAAGCCTGGCT |
| 1103 | GCCAGGCTTCTTGTATGTC | 1104 | GACATACAAGAAGCCTGGC |
| 1105 | CCAGGCTTCTTGTATGTCT | 1106 | AGACATACAAGAAGCCTGG |
| 1107 | CAGGCTTCTTGTATGTCTC | 1108 | GAGACATACAAGAAGCCTG |
| 1109 | AGGCTTCTTGTATGTCTCA | 1110 | TGAGACATACAAGAAGCCT |
| 1111 | GGCTTCTTGTATGTCTCAA | 1112 | TTGAGACATACAAGAAGCC |
| 1113 | GCTTCTTGTATGTCTCAAA | 1114 | TTTGAGACATACAAGAAGC |
| 1115 | CTTCTTGTATGTCTCAAAA | 1116 | TTTTGAGACATACAAGAAG |
| 1117 | TTCTTGTATGTCTCAAAAT | 1118 | ATTTTGAGACATACAAGAA |
| 1119 | TCTTGTATGTCTCAAAATG | 1120 | CATTTTGAGACATACAAGA |
| 1121 | CTTGTATGTCTCAAAATGC | 1122 | GCATTTTGAGACATACAAG |
| 1123 | TTGTATGTCTCAAAATGCC | 1124 | GGCATTTTGAGACATACAA |
| 1125 | TGTATGTCTCAAAATGCCA | 1126 | TGGCATTTTGAGACATACA |
| 1127 | GTATGTCTCAAAATGCCAG | 1128 | CTGGCATTTTGAGACATAC |
| 1129 | TATGTCTCAAAATGCCAGC | 1130 | GCTGGCATTTTGAGACATA |
| 1131 | ATGTCTCAAAATGCCAGCC | 1132 | GGCTGGCATTTTGAGACAT |
| 1133 | TGTCTCAAAATGCCAGCCT | 1134 | AGGCTGGCATTTTGAGACA |
| 1135 | GTCTCAAAATGCCAGCCTT | 1136 | AAGGCTGGCATTTTGAGAC |
| 1137 | TCTCAAAATGCCAGCCTTC | 1138 | GAAGGCTGGCATTTTGAGA |
| 1139 | CTCAAAATGCCAGCCTTCT | 1140 | AGAAGGCTGGCATTTTGAG |
| 1141 | TCAAAATGCCAGCCTTCTG | 1142 | CAGAAGGCTGGCATTTTGA |
| 1143 | CAAAATGCCAGCCTTCTGA | 1144 | TCAGAAGGCTGGCATTTTG |
| 1145 | AAAATGCCAGCCTTCTGAA | 1146 | TTCAGAAGGCTGGCATTTT |
| 1147 | AAATGCCAGCCTTCTGAAA | 1148 | TTTCAGAAGGCTGGCATTT |
| 1149 | AATGCCAGCCTTCTGAAAG | 1150 | CTTTCAGAAGGCTGGCATT |
| 1151 | ATGCCAGCCTTCTGAAAGT | 1152 | ACTTTCAGAAGGCTGGCAT |
| 1153 | TGCCAGCCTTCTGAAAGTA | 1154 | TACTTTCAGAAGGCTGGCA |
| 1155 | GCCAGCCTTCTGAAAGTAT | 1156 | ATACTTTCAGAAGGCTGGC |
| 1157 | CCAGCCTTCTGAAAGTATA | 1158 | TATACTTTCAGAAGGCTGG |
| 1159 | CAGCCTTCTGAAAGTATAC | 1160 | GTATACTTTCAGAAGGCTG |
| 1161 | AGCCTTCTGAAAGTATACA | 1162 | TGTATACTTTCAGAAGGCT |
| 1163 | GCCTTCTGAAAGTATACAG | 1164 | CTGTATACTTTCAGAAGGC |
| 1165 | CCTTCTGAAAGTATACAGC | 1166 | GCTGTATACTTTCAGAAGG |
| 1167 | CTTCTGAAAGTATACAGCA | 1168 | TGCTGTATACTTTCAGAAG |
| 1169 | TTCTGAAAGTATACAGCAA | 1170 | TTGCTGTATACTTTCAGAA |
| 1171 | TCTGAAAGTATACAGCAAA | 1172 | TTTGCTGTATACTTTCAGA |
| 1173 | CTGAAAGTATACAGCAAAG | 1174 | CTTTGCTGTATACTTTCAG |
| 1175 | TGAAAGTATACAGCAAAGA | 1176 | TCTTTGCTGTATACTTTCA |
| 1177 | GAAAGTATACAGCAAAGAG | 1178 | CTCTTTGCTGTATACTTTC |
| 1179 | AAAGTATACAGCAAAGAGG | 1180 | CCTCTTTGCTGTATACTTT |
| 1181 | AAGTATACAGCAAAGAGGA | 1182 | TCCTCTTTGCTGTATACTT |
| 1183 | AGTATACAGCAAAGAGGAC | 1184 | GTCCTCTTTGCTGTATACT |
| 1185 | GTATACAGCAAAGAGGACC | 1186 | GGTCCTCTTTGCTGTATAC |
| 1187 | TATACAGCAAAGAGGACCA | 1188 | TGGTCCTCTTTGCTGTATA |
| 1189 | ATACAGCAAAGAGGACCAG | 1190 | CTGGTCCTCTTTGCTGTAT |
| 1191 | TACAGCAAAGAGGACCAGG | 1192 | CCTGGTCCTCTTTGCTGTA |
| 1193 | ACAGCAAAGAGGACCAGGA | 1194 | TCCTGGTCCTCTTTGCTGT |
| 1195 | CAGCAAAGAGGACCAGGAT | 1196 | ATCCTGGTCCTCTTTGCTG |
| 1197 | AGCAAAGAGGACCAGGATT | 1198 | AATCCTGGTCCTCTTTGCT |
| 1199 | GCAAAGAGGACCAGGATTT | 1200 | AAATCCTGGTCCTCTTTGC |
| 1201 | CAAAGAGGACCAGGATTTA | 1202 | TAAATCCTGGTCCTCTTTG |
| 1203 | AAAGAGGACCAGGATTTAC | 1204 | GTAAATCCTGGTCCTCTTT |
| 1205 | AAGAGGACCAGGATTTACT | 1206 | AGTAAATCCTGGTCCTCTT |
| 1207 | AGAGGACCAGGATTTACTT | 1208 | AAGTAAATCCTGGTCCTCT |
| 1209 | GAGGACCAGGATTTACTTA | 1210 | TAAGTAAATCCTGGTCCTC |
| 1211 | AGGACCAGGATTTACTTAA | 1212 | TTAAGTAAATCCTGGTCCT |
| 1213 | GGACCAGGATTTACTTAAA | 1214 | TTTAAGTAAATCCTGGTCC |
| 1215 | GACCAGGATTTACTTAAAC | 1216 | GTTTAAGTAAATCCTGGTC |
| 1217 | ACCAGGATTTACTTAAACT | 1218 | AGTTTAAGTAAATCCTGGT |
| 1219 | CCAGGATTTACTTAAACTG | 1220 | CAGTTTAAGTAAATCCTGG |
| 1221 | CAGGATTTACTTAAACTGG | 1222 | CCAGTTTAAGTAAATCCTG |
| 1223 | AGGATTTACTTAAACTGGT | 1224 | ACCAGTTTAAGTAAATCCT |
| 1225 | GGATTTACTTAAACTGGTG | 1226 | CACCAGTTTAAGTAAATCC |
| 1227 | GATTTACTTAAACTGGTGA | 1228 | TCACCAGTTTAAGTAAATC |
| 1229 | ATTTACTTAAACTGGTGAA | 1230 | TTCACCAGTTTAAGTAAAT |
| 1231 | TTTACTTAAACTGGTGAAG | 1232 | CTTCACCAGTTTAAGTAAA |
| 1233 | TTACTTAAACTGGTGAAGT | 1234 | ACTTCACCAGTTTAAGTAA |
| 1235 | TACTTAAACTGGTGAAGTC | 1236 | GACTTCACCAGTTTAAGTA |
| 1237 | ACTTAAACTGGTGAAGTCA | 1238 | TGACTTCACCAGTTTAAGT |
| 1239 | CTTAAACTGGTGAAGTCAT | 1240 | ATGACTTCACCAGTTTAAG |
| 1241 | TTAAACTGGTGAAGTCATA | 1242 | TATGACTTCACCAGTTTAA |
| 1243 | TAAACTGGTGAAGTCATAT | 1244 | ATATGACTTCACCAGTTTA |
| 1245 | AAACTGGTGAAGTCATATC | 1246 | GATATGACTTCACCAGTTT |
| 1247 | AACTGGTGAAGTCATATCA | 1248 | TGATATGACTTCACCAGTT |
| 1249 | ACTGGTGAAGTCATATCAT | 1250 | ATGATATGACTTCACCAGT |
| 1251 | CTGGTGAAGTCATATCATT | 1252 | AATGATATGACTTCACCAG |
| 1253 | TGGTGAAGTCATATCATTG | 1254 | CAATGATATGACTTCACCA |
| 1255 | GGTGAAGTCATATCATTGG | 1256 | CCAATGATATGACTTCACC |
| 1257 | GTGAAGTCATATCATTGGA | 1258 | TCCAATGATATGACTTCAC |
| 1259 | TGAAGTCATATCATTGGAT | 1260 | ATCCAATGATATGACTTCA |
| 1261 | GAAGTCATATCATTGGATG | 1262 | CATCCAATGATATGACTTC |
| 1263 | AAGTCATATCATTGGATGG | 1264 | CCATCCAATGATATGACTT |
| 1265 | AGTCATATCATTGGATGGG | 1266 | CCCATCCAATGATATGACT |
| 1267 | GTCATATCATTGGATGGGA | 1268 | TCCCATCCAATGATATGAC |
| 1269 | TCATATCATTGGATGGGAC | 1270 | GTCCCATCCAATGATATGA |
| 1271 | CATATCATTGGATGGGACT | 1272 | AGTCCCATCCAATGATATG |
| 1273 | ATATCATTGGATGGGACTA | 1274 | TAGTCCCATCCAATGATAT |
| 1275 | TATCATTGGATGGGACTAG | 1276 | CTAGTCCCATCCAATGATA |
| 1277 | ATCATTGGATGGGACTAGT | 1278 | ACTAGTCCCATCCAATGAT |
| 1279 | TCATTGGATGGGACTAGTA | 1280 | TACTAGTCCCATCCAATGA |
| 1281 | CATTGGATGGGACTAGTAC | 1282 | GTACTAGTCCCATCCAATG |
| 1283 | ATTGGATGGGACTAGTACA | 1284 | TGTACTAGTCCCATCCAAT |
| 1285 | TTGGATGGGACTAGTACAC | 1286 | GTGTACTAGTCCCATCCAA |
| 1287 | TGGATGGGACTAGTACACA | 1288 | TGTGTACTAGTCCCATCCA |
| 1289 | GGATGGGACTAGTACACAT | 1290 | ATGTGTACTAGTCCCATCC |
| 1291 | GATGGGACTAGTACACATT | 1292 | AATGTGTACTAGTCCCATC |
| 1293 | ATGGGACTAGTACACATTC | 1294 | GAATGTGTACTAGTCCCAT |
| 1295 | TGGGACTAGTACACATTCC | 1296 | GGAATGTGTACTAGTCCCA |
| 1297 | GGGACTAGTACACATTCCA | 1298 | TGGAATGTGTACTAGTCCC |
| 1299 | GGACTAGTACACATTCCAA | 1300 | TTGGAATGTGTACTAGTCC |
| 1301 | GACTAGTACACATTCCAAC | 1302 | GTTGGAATGTGTACTAGTC |
| 1303 | ACTAGTACACATTCCAACA | 1304 | TGTTGGAATGTGTACTAGT |
| 1305 | CTAGTACACATTCCAACAA | 1306 | TTGTTGGAATGTGTACTAG |
| 1307 | TAGTACACATTCCAACAAA | 1308 | TTTGTTGGAATGTGTACTA |
| 1309 | AGTACACATTCCAACAAAT | 1310 | ATTTGTTGGAATGTGTACT |
| 1311 | GTACACATTCCAACAAATG | 1312 | CATTTGTTGGAATGTGTAC |
| 1313 | TACACATTCCAACAAATGG | 1314 | CCATTTGTTGGAATGTGTA |
| 1315 | ACACATTCCAACAAATGGA | 1316 | TCCATTTGTTGGAATGTGT |
| 1317 | CACATTCCAACAAATGGAT | 1318 | ATCCATTTGTTGGAATGTG |
| 1319 | ACATTCCAACAAATGGATC | 1320 | GATCCATTTGTTGGAATGT |
| 1321 | CATTCCAACAAATGGATCT | 1322 | AGATCCATTTGTTGGAATG |
| 1323 | ATTCCAACAAATGGATCTT | 1324 | AAGATCCATTTGTTGGAAT |
| 1325 | TTCCAACAAATGGATCTTG | 1326 | CAAGATCCATTTGTTGGAA |
| 1327 | TCCAACAAATGGATCTTGG | 1328 | CCAAGATCCATTTGTTGGA |
| 1329 | CCAACAAATGGATCTTGGC | 1330 | GCCAAGATCCATTTGTTGG |
| 1331 | CAACAAATGGATCTTGGCA | 1332 | TGCCAAGATCCATTTGTTG |
| 1333 | AACAAATGGATCTTGGCAG | 1334 | CTGCCAAGATCCATTTGTT |
| 1335 | ACAAATGGATCTTGGCAGT | 1336 | ACTGCCAAGATCCATTTGT |
| 1337 | CAAATGGATCTTGGCAGTG | 1338 | CACTGCCAAGATCCATTTG |
| 1339 | AAATGGATCTTGGCAGTGG | 1340 | CCACTGCCAAGATCCATTT |
| 1341 | AATGGATCTTGGCAGTGGG | 1342 | CCCACTGCCAAGATCCATT |
| 1343 | ATGGATCTTGGCAGTGGGA | 1344 | TCCCACTGCCAAGATCCAT |
| 1345 | TGGATCTTGGCAGTGGGAA | 1346 | TTCCCACTGCCAAGATCCA |
| 1347 | GGATCTTGGCAGTGGGAAG | 1348 | CTTCCCACTGCCAAGATCC |
| 1349 | GATCTTGGCAGTGGGAAGA | 1350 | TCTTCCCACTGCCAAGATC |
| 1351 | ATCTTGGCAGTGGGAAGAT | 1352 | ATCTTCCCACTGCCAAGAT |
| 1353 | TCTTGGCAGTGGGAAGATG | 1354 | CATCTTCCCACTGCCAAGA |
| 1355 | CTTGGCAGTGGGAAGATGG | 1356 | CCATCTTCCCACTGCCAAG |
| 1357 | TTGGCAGTGGGAAGATGGC | 1358 | GCCATCTTCCCACTGCCAA |
| 1359 | TGGCAGTGGGAAGATGGCT | 1360 | AGCCATCTTCCCACTGCCA |
| 1361 | GGCAGTGGGAAGATGGCTC | 1362 | GAGCCATCTTCCCACTGCC |
| 1363 | GCAGTGGGAAGATGGCTCC | 1364 | GGAGCCATCTTCCCACTGC |
| 1365 | CAGTGGGAAGATGGCTCCA | 1366 | TGGAGCCATCTTCCCACTG |
| 1367 | AGTGGGAAGATGGCTCCAT | 1368 | ATGGAGCCATCTTCCCACT |
| 1369 | GTGGGAAGATGGCTCCATT | 1370 | AATGGAGCCATCTTCCCAC |
| 1371 | TGGGAAGATGGCTCCATTC | 1372 | GAATGGAGCCATCTTCCCA |
| 1373 | GGGAAGATGGCTCCATTCT | 1374 | AGAATGGAGCCATCTTCCC |
| 1375 | GGAAGATGGCTCCATTCTC | 1376 | GAGAATGGAGCCATCTTCC |
| 1377 | GAAGATGGCTCCATTCTCT | 1378 | AGAGAATGGAGCCATCTTC |
| 1379 | AAGATGGCTCCATTCTCTC | 1380 | GAGAGAATGGAGCCATCTT |
| 1381 | AGATGGCTCCATTCTCTCA | 1382 | TGAGAGAATGGAGCCATCT |
| 1383 | GATGGCTCCATTCTCTCAC | 1384 | GTGAGAGAATGGAGCCATC |
| 1385 | ATGGCTCCATTCTCTCACC | 1386 | GGTGAGAGAATGGAGCCAT |
| 1387 | TGGCTCCATTCTCTCACCC | 1388 | GGGTGAGAGAATGGAGCCA |
| 1389 | GGCTCCATTCTCTCACCCA | 1390 | TGGGTGAGAGAATGGAGCC |
| 1391 | GCTCCATTCTCTCACCCAA | 1392 | TTGGGTGAGAGAATGGAGC |
| 1393 | CTCCATTCTCTCACCCAAC | 1394 | GTTGGGTGAGAGAATGGAG |
| 1395 | TCCATTCTCTCACCCAACC | 1396 | GGTTGGGTGAGAGAATGGA |
| 1397 | CCATTCTCTCACCCAACCT | 1398 | AGGTTGGGTGAGAGAATGG |
| 1399 | CATTCTCTCACCCAACCTA | 1400 | TAGGTTGGGTGAGAGAATG |
| 1401 | ATTCTCTCACCCAACCTAC | 1402 | GTAGGTTGGGTGAGAGAAT |
| 1403 | TTCTCTCACCCAACCTACT | 1404 | AGTAGGTTGGGTGAGAGAA |
| 1405 | TCTCTCACCCAACCTACTA | 1406 | TAGTAGGTTGGGTGAGAGA |
| 1407 | CTCTCACCCAACCTACTAA | 1408 | TTAGTAGGTTGGGTGAGAG |
| 1409 | TCTCACCCAACCTACTAAC | 1410 | GTTAGTAGGTTGGGTGAGA |
| 1411 | CTCACCCAACCTACTAACA | 1412 | TGTTAGTAGGTTGGGTGAG |
| 1413 | TCACCCAACCTACTAACAA | 1414 | TTGTTAGTAGGTTGGGTGA |
| 1415 | CACCCAACCTACTAACAAT | 1416 | ATTGTTAGTAGGTTGGGTG |
| 1417 | ACCCAACCTACTAACAATA | 1418 | TATTGTTAGTAGGTTGGGT |
| 1419 | CCCAACCTACTAACAATAA | 1420 | TTATTGTTAGTAGGTTGGG |
| 1421 | CCAACCTACTAACAATAAT | 1422 | ATTATTGTTAGTAGGTTGG |
| 1423 | CAACCTACTAACAATAATT | 1424 | AATTATTGTTAGTAGGTTG |
| 1425 | AACCTACTAACAATAATTG | 1426 | CAATTATTGTTAGTAGGTT |
| 1427 | ACCTACTAACAATAATTGA | 1428 | TCAATTATTGTTAGTAGGT |
| 1429 | CCTACTAACAATAATTGAA | 1430 | TTCAATTATTGTTAGTAGG |
| 1431 | CTACTAACAATAATTGAAA | 1432 | TTTCAATTATTGTTAGTAG |
| 1433 | TACTAACAATAATTGAAAT | 1434 | ATTTCAATTATTGTTAGTA |
| 1435 | ACTAACAATAATTGAAATG | 1436 | CATTTCAATTATTGTTAGT |
| 1437 | CTAACAATAATTGAAATGC | 1438 | GCATTTCAATTATTGTTAG |
| 1439 | TAACAATAATTGAAATGCA | 1440 | TGCATTTCAATTATTGTTA |
| 1441 | AACAATAATTGAAATGCAG | 1442 | CTGCATTTCAATTATTGTT |
| 1443 | ACAATAATTGAAATGCAGA | 1444 | TCTGCATTTCAATTATTGT |
| 1445 | CAATAATTGAAATGCAGAA | 1446 | TTCTGCATTTCAATTATTG |
| 1447 | AATAATTGAAATGCAGAAG | 1448 | CTTCTGCATTTCAATTATT |
| 1449 | ATAATTGAAATGCAGAAGG | 1450 | CCTTCTGCATTTCAATTAT |
| 1451 | TAATTGAAATGCAGAAGGG | 1452 | CCCTTCTGCATTTCAATTA |
| 1453 | AATTGAAATGCAGAAGGGA | 1454 | TCCCTTCTGCATTTCAATT |
| 1455 | ATTGAAATGCAGAAGGGAG | 1456 | CTCCCTTCTGCATTTCAAT |
| 1457 | TTGAAATGCAGAAGGGAGA | 1458 | TCTCCCTTCTGCATTTCAA |
| 1459 | TGAAATGCAGAAGGGAGAC | 1460 | GTCTCCCTTCTGCATTTCA |
| 1461 | GAAATGCAGAAGGGAGACT | 1462 | AGTCTCCCTTCTGCATTTC |
| 1463 | AAATGCAGAAGGGAGACTG | 1464 | CAGTCTCCCTTCTGCATTT |
| 1465 | AATGCAGAAGGGAGACTGT | 1466 | ACAGTCTCCCTTCTGCATT |
| 1467 | ATGCAGAAGGGAGACTGTG | 1468 | CACAGTCTCCCTTCTGCAT |
| 1469 | TGCAGAAGGGAGACTGTGC | 1470 | GCACAGTCTCCCTTCTGCA |
| 1471 | GCAGAAGGGAGACTGTGCA | 1472 | TGCACAGTCTCCCTTCTGC |
| 1473 | CAGAAGGGAGACTGTGCAC | 1474 | GTGCACAGTCTCCCTTCTG |
| 1475 | AGAAGGGAGACTGTGCACT | 1476 | AGTGCACAGTCTCCCTTCT |
| 1477 | GAAGGGAGACTGTGCACTC | 1478 | GAGTGCACAGTCTCCCTTC |
| 1479 | AAGGGAGACTGTGCACTCT | 1480 | AGAGTGCACAGTCTCCCTT |
| 1481 | AGGGAGACTGTGCACTCTA | 1482 | TAGAGTGCACAGTCTCCCT |
| 1483 | GGGAGACTGTGCACTCTAT | 1484 | ATAGAGTGCACAGTCTCCC |
| 1485 | GGAGACTGTGCACTCTATG | 1486 | CATAGAGTGCACAGTCTCC |
| 1487 | GAGACTGTGCACTCTATGC | 1488 | GCATAGAGTGCACAGTCTC |
| 1489 | AGACTGTGCACTCTATGCC | 1490 | GGCATAGAGTGCACAGTCT |
| 1491 | GACTGTGCACTCTATGCCT | 1492 | AGGCATAGAGTGCACAGTC |
| 1493 | ACTGTGCACTCTATGCCTC | 1494 | GAGGCATAGAGTGCACAGT |
| 1495 | CTGTGCACTCTATGCCTCG | 1496 | CGAGGCATAGAGTGCACAG |
| 1497 | TGTGCACTCTATGCCTCGA | 1498 | TCGAGGCATAGAGTGCACA |
| 1499 | GTGCACTCTATGCCTCGAG | 1500 | CTCGAGGCATAGAGTGCAC |
| 1501 | TGCACTCTATGCCTCGAGC | 1502 | GCTCGAGGCATAGAGTGCA |
| 1503 | GCACTCTATGCCTCGAGCT | 1504 | AGCTCGAGGCATAGAGTGC |
| 1505 | CACTCTATGCCTCGAGCTT | 1506 | AAGCTCGAGGCATAGAGTG |
| 1507 | ACTCTATGCCTCGAGCTTT | 1508 | AAAGCTCGAGGCATAGAGT |
| 1509 | CTCTATGCCTCGAGCTTTA | 1510 | TAAAGCTCGAGGCATAGAG |
| 1511 | TCTATGCCTCGAGCTTTAA | 1512 | TTAAAGCTCGAGGCATAGA |
| 1513 | CTATGCCTCGAGCTTTAAA | 1514 | TTTAAAGCTCGAGGCATAG |
| 1515 | TATGCCTCGAGCTTTAAAG | 1516 | CTTTAAAGCTCGAGGCATA |
| 1517 | ATGCCTCGAGCTTTAAAGG | 1518 | CCTTTAAAGCTCGAGGCAT |
| 1519 | TGCCTCGAGCTTTAAAGGC | 1520 | GCCTTTAAAGCTCGAGGCA |
| 1521 | GCCTCGAGCTTTAAAGGCT | 1522 | AGCCTTTAAAGCTCGAGGC |
| 1523 | CCTCGAGCTTTAAAGGCTA | 1524 | TAGCCTTTAAAGCTCGAGG |
| 1525 | CTCGAGCTTTAAAGGCTAT | 1526 | ATAGCCTTTAAAGCTCGAG |
| 1527 | TCGAGCTTTAAAGGCTATA | 1528 | TATAGCCTTTAAAGCTCGA |
| 1529 | CGAGCTTTAAAGGCTATAT | 1530 | ATATAGCCTTTAAAGCTCG |
| 1531 | GAGCTTTAAAGGCTATATA | 1532 | TATATAGCCTTTAAAGCTC |
| 1533 | AGCTTTAAAGGCTATATAG | 1534 | CTATATAGCCTTTAAAGCT |
| 1535 | GCTTTAAAGGCTATATAGA | 1536 | TCTATATAGCCTTTAAAGC |
| 1537 | CTTTAAAGGCTATATAGAA | 1538 | TTCTATATAGCCTTTAAAG |
| 1539 | TTTAAAGGCTATATAGAAA | 1540 | TTTCTATATAGCCTTTAAA |
| 1541 | TTAANGGCTATATAGAAAA | 1542 | TTTTCTATATAGCCTTTAA |
| 1543 | TAAAGGCTATATAGAAAAC | 1544 | GTTTTCTATATAGCCTTTA |
| 1545 | AAAGGCTATATAGAAAACT | 1546 | AGTTTTCTATATAGCCTTT |
| 1547 | AAGGCTATATAGAAAACTG | 1548 | CAGTTTTCTATATAGCCTT |
| 1549 | AGGCTATATAGAAAACTGT | 1550 | ACAGTTTTCTATATAGCCT |
| 1551 | GGCTATATAGAAAACTGTT | 1552 | AACAGTTTTCTATATAGCC |
| 1553 | GCTATATAGAAAACTGTTC | 1554 | GAACAGTTTTCTATATAGC |
| 1555 | CTATATAGAAAACTGTTCA | 1556 | TGAACAGTTTTCTATATAG |
| 1557 | TATATAGAAAACTGTTCAA | 1558 | TTGAACAGTTTTCTATATA |
| 1559 | ATATAGAAAACTGTTCAAC | 1560 | GTTGAACAGTTTTCTATAT |
| 1561 | TATAGAAAACTGTTCAACT | 1562 | AGTTGAACAGTTTTCTATA |
| 1563 | ATAGAAAACTGTTCAACTC | 1564 | GAGTTGAACAGTTTTCTAT |
| 1565 | TAGAAAACTGTTCAACTCC | 1566 | GGAGTTGAACAGTTTTCTA |
| 1567 | AGAAAACTGTTCAACTCCA | 1568 | TGGAGTTGAACAGTTTTCT |
| 1569 | GAAAACTGTTCAACTCCAA | 1570 | TTGGAGTTGAACAGTTTTC |
| 1571 | AAAACTGTTCAACTCCAAA | 1572 | TTTGGAGTTGAACAGTTTT |
| 1573 | AAACTGTTCAACTCCAAAT | 1574 | ATTTGGAGTTGAACAGTTT |
| 1575 | AACTGTTCAACTCCAAATA | 1576 | TATTTGGAGTTGAACAGTT |
| 1577 | ACTGTTCAACTCCAAATAC | 1578 | GTATTTGGAGTTGAACAGT |
| 1579 | CTGTTCAACTCCAAATACG | 1580 | CGTATTTGGAGTTGAACAG |
| 1581 | TGTTCAACTCCAAATACGT | 1582 | ACGTATTTGGAGTTGAACA |
| 1583 | GTTCAACTCCAAATACGTA | 1584 | TACGTATTTGGAGTTGAAC |
| 1585 | TTCAACTCCAAATACGTAC | 1586 | GTACGTATTTGGAGTTGAA |
| 1587 | TCAACTCCAAATACGTACA | 1588 | TGTACGTATTTGGAGTTGA |
| 1589 | CAACTCCAAATACGTACAT | 1590 | ATGTACGTATTTGGAGTTG |
| 1591 | AACTCCAAATACGTACATC | 1592 | GATGTACGTATTTGGAGTT |
| 1593 | ACTCCAAATACGTACATCT | 1594 | AGATGTACGTATTTGGAGT |
| 1595 | CTCCAAATACGTACATCTG | 1596 | CAGATGTACGTATTTGGAG |
| 1597 | TCCAAATACGTACATCTGC | 1598 | GCAGATGTACGTATTTGGA |
| 1599 | CCAAATACGTACATCTGCA | 1600 | TGCAGATGTACGTATTTGG |
| 1601 | CAAATACGTACATCTGCAT | 1602 | ATGCAGATGTACGTATTTG |
| 1603 | AAATACGTACATCTGCATG | 1604 | CATGCAGATGTACGTATTT |
| 1605 | AATACGTACATCTGCATGC | 1606 | GCATGCAGATGTACGTATT |
| 1607 | ATACGTACATCTGCATGCA | 1608 | TGCATGCAGATGTACGTAT |
| 1609 | TACGTACATCTGCATGCAA | 1610 | TTGCATGCAGATGTACGTA |
| 1611 | ACGTACATCTGCATGCAAA | 1612 | TTTGCATGCAGATGTACGT |
| 1613 | CGTACATCTGCATGCAAAG | 1614 | CTTTGCATGCAGATGTACG |
| 1615 | GTACATCTGCATGCAAAGG | 1616 | CCTTTGCATGCAGATGTAC |
| 1617 | TACATCTGCATGCAAAGGA | 1618 | TCCTTTGCATGCAGATGTA |
| 1619 | ACATCTGCATGCAAAGGAC | 1620 | GTCCTTTGCATGCAGATGT |
| 1621 | CATCTGCATGCAAAGGACT | 1622 | AGTCCTTTGCATGCAGATG |
| 1623 | ATCTGCATGCAAAGGACTG | 1624 | CAGTCCTTTGCATGCAGAT |
| 1625 | TCTGCATGCAAAGGACTGT | 1626 | ACAGTCCTTTGCATGCAGA |
| 1627 | CTGCATGCAAAGGACTGTG | 1628 | CACAGTCCTTTGCATGCAG |
| 1629 | TGCATGCAAAGGACTGTGT | 1630 | ACACAGTCCTTTGCATGCA |
| 1631 | GCATGCAAAGGACTGTGTA | 1632 | TACACAGTCCTTTGCATGC |
| 1633 | CATGCAAAGGACTGTGTAA | 1634 | TTACACAGTCCTTTGCATG |
| 1635 | ATGCAAAGGACTGTGTAAA | 1636 | TTTACACAGTCCTTTGCAT |
| 1637 | TGCAAAGGACTGTGTAAAG | 1638 | CTTTACACAGTCCTTTGCA |
| 1639 | GCAAAGGACTGTGTAAAGA | 1640 | TCTTTACACAGTCCTTTGC |
| 1641 | CAAAGGACTGTGTAAAGAT | 1642 | ATCTTTACACAGTCCTTTG |
| 1643 | AAAGGACTGTGTAAAGATG | 1644 | CATCTTTACACAGTCCTTT |
| 1645 | AAGGACTGTGTAAAGATGA | 1646 | TCATCTTTACACAGTCCTT |
| 1647 | AGGACTGTGTAAAGATGAT | 1648 | ATCATCTTTACACAGTCCT |
| 1649 | GGACTGTGTAAAGATGATC | 1650 | GATCATCTTTACACAGTCC |
| 1651 | GACTGTGTAAAGATGATCA | 1652 | TGATCATCTTTACACAGTC |
| 1653 | ACTGTGTAAAGATGATCAA | 1654 | TTGATCATCTTTACACAGT |
| 1655 | CTGTGTAAAGATGATCAAC | 1656 | GTTGATCATCTTTACACAG |
| 1657 | TGTGTAAAGATGATCAACC | 1658 | GGTTGATCATCTTTACACA |
| 1659 | GTGTAAAGATGATCAACCA | 1660 | TGGTTGATCATCTTTACAC |
| 1661 | TGTAAAGATGATCAACCAT | 1662 | ATGGTTGATCATCTTTACA |
| 1663 | GTAAAGATGATCAACCATC | 1664 | GATGGTTGATCATCTTTAC |
| 1665 | TAAAGATGATCAACCATCT | 1666 | AGATGGTTGATCATCTTTA |
| 1667 | AAAGATGATCAACCATCTC | 1668 | GAGATGGTTGATCATCTTT |
| 1669 | AAGATGATCAACCATCTCA | 1670 | TGAGATGGTTGATCATCTT |
| 1671 | AGATGATCAACCATCTCAA | 1672 | TTGAGATGGTTGATCATCT |
| 1673 | GATGATCAACCATCTCAAT | 1674 | ATTGAGATGGTTGATCATC |
| 1675 | ATGATCAACCATCTCAATA | 1676 | TATTGAGATGGTTGATCAT |
| 1677 | TGATCAACCATCTCAATAA | 1678 | TTATTGAGATGGTTGATCA |
| 1679 | GATCAACCATCTCAATAAA | 1680 | TTTATTGAGATGGTTGATC |
| 1681 | ATCAACCATCTCAATAAAA | 1682 | TTTTATTGAGATGGTTGAT |
| 1683 | TCAACCATCTCAATAAAAG | 1684 | CTTTTATTGAGATGGTTGA |
| 1685 | CAACCATCTCAATAAAAGC | 1686 | GCTTTTATTGAGATGGTTG |
| 1687 | AACCATCTCAATAAAAGCC | 1688 | GGCTTTTATTGAGATGGTT |
| 1689 | ACCATCTCAATAAAAGCCA | 1690 | TGGCTTTTATTGAGATGGT |
| 1691 | CCATCTCAATAAAAGCCAG | 1692 | CTGGCTTTTATTGAGATGG |
| 1693 | CATCTCAATAAAAGCCAGG | 1694 | CCTGGCTTTTATTGAGATG |
| 1695 | ATCTCAATAAAAGCCAGGA | 1696 | TCCTGGCTTTTATTGAGAT |
| 1697 | TCTCAATAAAAGCCAGGAA | 1698 | TTCCTGGCTTTTATTGAGA |
| 1699 | CTCAATAAAAGCCAGGAAC | 1700 | GTTCCTGGCTTTTATTGAG |
| 1701 | TCAATAAAAGCCAGGAACA | 1702 | TGTTCCTGGCTTTTATTGA |
| 1703 | CAATAAAAGCCAGGAACAG | 1704 | CTGTTCCTGGCTTTTATTG |
| 1705 | AATAAAAGCCAGGAACAGA | 1706 | TCTGTTCCTGGCTTTTATT |
| 1707 | ATAAAAGCCAGGAACAGAG | 1708 | CTCTGTTCCTGGCTTTTAT |
| 1709 | TAAAAGCCAGGAACAGAGA | 1710 | TCTCTGTTCCTGGCTTTTA |
| 1711 | AAAAGCCAGGAACAGAGAA | 1712 | TTCTCTGTTCCTGGCTTTT |
| 1713 | AAAGCCAGGAACAGAGAAG | 1714 | CTTCTCTGTTCCTGGCTTT |
| 1715 | AAGCCAGGAACAGAGAAGA | 1716 | TCTTCTCTGTTCCTGGCTT |
| 1717 | AGCCAGGAACAGAGAAGAG | 1718 | CTCTTCTCTGTTCCTGGCT |
| 1719 | GCCAGGAACAGAGAAGAGA | 1720 | TCTCTTCTCTGTTCCTGGC |
| 1721 | CCAGGAACAGAGAAGAGAT | 1722 | ATCTCTTCTCTGTTCCTGG |
| 1723 | CAGGAACAGAGAAGAGATT | 1724 | AATCTCTTCTCTGTTCCTG |
| 1725 | AGGAACAGAGAAGAGATTA | 1726 | TAATCTCTTCTCTGTTCCT |
| 1727 | GGAACAGAGAAGAGATTAC | 1728 | GTAATCTCTTCTCTGTTCC |
| 1729 | GAACAGAGAAGAGATTACA | 1730 | TGTAATCTCTTCTCTGTTC |
| 1731 | AACAGAGAAGAGATTACAC | 1732 | GTGTAATCTCTTCTCTGTT |
| 1733 | ACAGAGAAGAGATTACACC | 1734 | GGTGTAATCTCTTCTCTGT |
| 1735 | CAGAGAAGAGATTACACCA | 1736 | TGGTGTAATCTCTTCTCTG |
| 1737 | AGAGAAGAGATTACACCAG | 1738 | CTGGTGTAATCTCTTCTCT |
| 1739 | GAGAAGAGATTACACCAGC | 1740 | GCTGGTGTAATCTCTTCTC |
| 1741 | AGAAGAGATTACACCAGCG | 1742 | CGCTGGTGTAATCTCTTCT |
| 1743 | GAAGAGATTACACCAGCGG | 1744 | CCGCTGGTGTAATCTCTTC |
| 1745 | AAGAGATTACACCAGCGGT | 1746 | ACCGCTGGTGTAATCTCTT |
| 1747 | AGAGATTACACCAGCGGTA | 1748 | TACCGCTGGTGTAATCTCT |
| 1749 | GAGATTACACCAGCGGTAA | 1750 | TTACCGCTGGTGTAATCTC |
| 1751 | AGATTACACCAGCGGTAAC | 1752 | GTTACCGCTGGTGTAATCT |
| 1753 | GATTACACCAGCGGTAACA | 1754 | TGTTACCGCTGGTGTAATC |
| 1755 | ATTACACCAGCGGTAACAC | 1756 | GTGTTACCGCTGGTGTAAT |
| 1757 | TTACACCAGCGGTAACACT | 1758 | AGTGTTACCGCTGGTGTAA |
| 1759 | TACACCAGCGGTAACACTG | 1760 | CAGTGTTACCGCTGGTGTA |
| 1761 | ACACCAGCGGTAACACTGC | 1762 | GCAGTGTTACCGCTGGTGT |
| 1763 | CACCAGCGGTAACACTGCC | 1764 | GGCAGTGTTACCGCTGGTG |
| 1765 | ACCAGCGGTAACACTGCCA | 1766 | TGGCAGTGTTACCGCTGGT |
| 1767 | CCAGCGGTAACACTGCCAA | 1768 | TTGGCAGTGTTACCGCTGG |
| 1769 | CAGCGGTAACACTGCCAAC | 1770 | GTTGGCAGTGTTACCGCTG |
| 1771 | AGCGGTAACACTGCCAACT | 1772 | AGTTGGCAGTGTTACCGCT |
| 1773 | GCGGTAACACTGCCAACTG | 1774 | CAGTTGGCAGTGTTACCGC |
| 1775 | CGGTAACACTGCCAACTGA | 1776 | TCAGTTGGCAGTGTTACCG |
| 1777 | GGTAACACTGCCAACTGAG | 1778 | CTCAGTTGGCAGTGTTACC |
| 1779 | GTAACACTGCCAACTGAGA | 1780 | TCTCAGTTGGCAGTGTTAC |
| 1781 | TAACACTGCCAACTGAGAC | 1782 | GTCTCAGTTGGCAGTGTTA |
| 1783 | AACACTGCCAACTGAGACT | 1784 | AGTCTCAGTTGGCAGTGTT |
| 1785 | ACACTGCCAACTGAGACTA | 1786 | TAGTCTCAGTTGGCAGTGT |
| 1787 | CACTGCCAACTGAGACTAA | 1788 | TTAGTCTCAGTTGGCAGTG |
| 1789 | ACTGCCAACTGAGACTAAA | 1790 | TTTAGTCTCAGTTGGCAGT |
| 1791 | CTGCCAACTGAGACTAAAG | 1792 | CTTTAGTCTCAGTTGGCAG |
| 1793 | TGCCAACTGAGACTAAAGG | 1794 | CCTTTAGTCTCAGTTGGCA |
| 1795 | GCCAACTGAGACTAAAGGA | 1796 | TCCTTTAGTCTCAGTTGGC |
| 1797 | CCAACTGAGACTAAAGGAA | 1798 | TTCCTTTAGTCTCAGTTGG |
| 1799 | CAACTGAGACTAAAGGAAA | 1800 | TTTCCTTTAGTCTCAGTTG |
| 1801 | AACTGAGACTAAAGGAAAC | 1802 | GTTTCCTTTAGTCTCAGTT |
| 1803 | ACTGAGACTAAAGGAAACA | 1804 | TGTTTCCTTTAGTCTCAGT |
| 1805 | CTGAGACTAAAGGAAACAA | 1806 | TTGTTTCCTTTAGTCTCAG |
| 1807 | TGAGACTAAAGGAAACAAA | 1808 | TTTGTTTCCTTTAGTCTCA |
| 1809 | GAGACTAAAGGAAACAAAC | 1810 | GTTTGTTTCCTTTAGTCTC |
| 1811 | AGACTAAAGGAAACAAACA | 1812 | TGTTTGTTTCCTTTAGTCT |
| 1813 | GACTAAAGGAAACAAACAA | 1814 | TTGTTTGTTTCCTTTAGTC |
| 1815 | ACTAAAGGAAACAAACAAA | 1816 | TTTGTTTGTTTCCTTTAGT |
| 1817 | CTAAAGGAAACAAACAAAA | 1818 | TTTTGTTTGTTTCCTTTAG |
| 1819 | TAAAGGAAACAAACAAAAA | 1820 | TTTTTGTTTGTTTCCTTTA |
| 1821 | AAAGGAAACAAACAAAAAC | 1822 | GTTTTTGTTTGTTTCCTTT |
| 1823 | AAGGAAACAAACAAAAACA | 1824 | TGTTTTTGTTTGTTTCCTT |
| 1825 | AGGAAACAAACAAAAACAG | 1826 | CTGTTTTTGTTTGTTTCCT |
| 1827 | GGAAACAAACAAAAACAGG | 1828 | CCTGTTTTTGTTTGTTTCC |
| 1829 | GAAACAAACAAAAACAGGA | 1830 | TCCTGTTTTTGTTTGTTTC |
| 1831 | AAACAAACAAAAACAGGAC | 1832 | GICCTGITITTGTTTGTTT |
| 1833 | AACAAACAAAAACAGGACA | 1834 | TGTCCTGTTTTTGTTTGTT |
| 1835 | ACAAACAAAAACAGGACAA | 1836 | TTGTCCTGTTTTTGTTTGT |
| 1837 | CAAACAAAAACAGGACAAA | 1838 | TTTGTCCTGTTTTTGTTTG |
| 1839 | AAACAAAAACAGGACAAAA | 1840 | TTTTGTCCTGTTTTTGTTT |
| 1841 | AACAAAAACAGGACAAAAT | 1842 | ATTTTGTCCTGTTTTTGTT |
| 1843 | ACAAAAACAGGACAAAATG | 1844 | CATTTTGTCCTGTTTTTGT |
| 1845 | CAAAAACAGGACAAAATGA | 1846 | TCATTTTGTCCTGTTTTTG |
| 1847 | AAAAACAGGACAAAATGAC | 1848 | GTCATTTTGTCCTGTTTTT |
| 1849 | AAAACAGGACAAAATGACC | 1850 | GGTCATTTTGTCCTGTTTT |
| 1851 | AAACAGGACAAAATGACCA | 1852 | TGGTCATTTTGTCCTGTTT |
| 1853 | AACAGGACAAAATGACCAA | 1854 | TTGGTCATTTTGTCCTGTT |
| 1855 | ACAGGACAAAATGACCAAA | 1856 | TTTGGTCATTTTGTCCTGT |
| 1857 | CAGGACAAAATGACCAAAG | 1858 | CTTTGGTCATTTTGTCCTG |
| 1859 | AGGACAAAATGACCAAAGA | 1860 | TCTTTGGTCATTTTGTCCT |
| 1861 | GGACAAAATGACCAAAGAC | 1862 | GTCTTTGGTCATTTTGTCC |
| 1863 | GACAAAATGACCAAAGACT | 1864 | AGTCTTTGGTCATTTTGTC |
| 1865 | ACAAAATGACCAAAGACTG | 1866 | CAGTCTTTGGTCATTTTGT |
| 1867 | CAAAATGACCAAAGACTGT | 1868 | ACAGTCTTTGGTCATTTTG |
| 1869 | AAAATGACCAAAGACTGTC | 1870 | GACAGTCTTTGGTCATTTT |
| 1871 | AAATGACCAAAGACTGTCA | 1872 | TGACAGTCTTTGGTCATTT |
| 1873 | AATGACCAAAGACTGTCAG | 1874 | CTGACAGTCTTTGGTCATT |
| 1875 | ATGACCAAAGACTGTCAGA | 1876 | TCTGACAGTCTTTGGTCAT |
| 1877 | TGACCAAAGACTGTCAGAT | 1878 | ATCTGACAGTCTTTGGTCA |
| 1879 | GACCAAAGACTGTCAGATT | 1880 | AATCTGACAGTCTTTGGTC |
| 1881 | ACCAAAGACTGTCAGATTT | 1882 | AAATCTGACAGTCTTTGGT |
| 1883 | CCAAAGACTGTCAGATTTC | 1884 | GAAATCTGACAGTCTTTGG |
| 1885 | CAAAGACTGTCAGATTTCT | 1886 | AGAAATCTGACAGTCTTTG |
| 1887 | AAAGACTGTCAGATTTCTT | 1888 | AAGAAATCTGACAGTCTTT |
| 1889 | AAGACTGTCAGATTTCTTA | 1890 | TAAGAAATCTGACAGTCTT |
| 1891 | AGACTGTCAGATTTCTTAG | 1892 | CTAAGAAATCTGACAGTCT |
| 1893 | GACTGTCAGATTTCTTAGA | 1894 | TCTAAGAAATCTGACAGTC |
| 1895 | ACTGTCAGATTTCTTAGAC | 1896 | GTCTAAGAAATCTGACAGT |
| 1897 | CTGTCAGATTTCTTAGACT | 1898 | AGTCTAAGAAATCTGACAG |
| 1899 | TGTCAGATTTCTTAGACTC | 1900 | GAGTCTAAGAAATCTGACA |
| 1901 | GTCAGATTTCTTAGACTCC | 1902 | GGAGTCTAAGAAATCTGAC |
| 1903 | TCAGATTTCTTAGACTCCA | 1904 | TGGAGTCTAAGAAATCTGA |
| 1905 | CAGATTTCTTAGACTCCAC | 1906 | GTGGAGTCTAAGAAATCTG |
| 1907 | AGATTTCTTAGACTCCACA | 1908 | TGTGGAGTCTAAGAAATCT |
| 1909 | GATTTCTTAGACTCCACAG | 1910 | CTGTGGAGTCTAAGAAATC |
| 1911 | ATTTCTTAGACTCCACAGG | 1912 | CCTGTGGAGTCTAAGAAAT |
| 1913 | TTTCTTAGACTCCACAGGA | 1914 | TCCTGTGGAGTCTAAGAAA |
| 1915 | TTCTTAGACTCCACAGGAC | 1916 | GTCCTGTGGAGTCTAAGAA |
| 1917 | TCTTAGACTCCACAGGACC | 1918 | GGTCCTGTGGAGTCTAAGA |
| 1919 | CTTAGACTCCACAGGACCA | 1920 | TGGTCCTGTGGAGTCTAAG |
| 1921 | TTAGACTCCACAGGACCAA | 1922 | TTGGTCCTGTGGAGTCTAA |
| 1923 | TAGACTCCACAGGACCAAA | 1924 | TTTGGTCCTGTGGAGTCTA |
| 1925 | AGACTCCACAGGACCAAAC | 1926 | GTTTGGTCCTGTGGAGTCT |
| 1927 | GACTCCACAGGACCAAACC | 1928 | GGTTTGGTCCTGTGGAGTC |
| 1929 | ACTCCACAGGACCAAACCA | 1930 | TGGTTTGGTCCTGTGGAGT |
| 1931 | CTCCACAGGACCAAACCAT | 1932 | ATGGTTTGGTCCTGTGGAG |
| 1933 | TCCACAGGACCAAACCATA | 1934 | TATGGTTTGGTCCTGTGGA |
| 1935 | CCACAGGACCAAACCATAG | 1936 | CTATGGTTTGGTCCTGTGG |
| 1937 | CACAGGACCAAACCATAGA | 1938 | TCTATGGTTTGGTCCTGTG |
| 1939 | ACAGGACCAAACCATAGAA | 1940 | TTCTATGGTTTGGTCCTGT |
| 1941 | CAGGACCAAACCATAGAAC | 1942 | GTTCTATGGTTTGGTCCTG |
| 1943 | AGGACCAAACCATAGAACA | 1944 | TGTTCTATGGTTTGGTCCT |
| 1945 | GGACCAAACCATAGAACAA | 1946 | TTGTTCTATGGTTTGGTCC |
| 1947 | GACCAAACCATAGAACAAT | 1948 | ATTGTTCTATGGTTTGGTC |
| 1949 | ACCAAACCATAGAACAATT | 1950 | AATTGTTCTATGGTTTGGT |
| 1951 | CCAAACCATAGAACAATTT | 1952 | AAATTGTTCTATGGTTTGG |
| 1953 | CAAACCATAGAACAATTTC | 1954 | GAAATTGTTCTATGGTTTG |
| 1955 | AAACCATAGAACAATTTCA | 1956 | TGAAATTGTTCTATGGTTT |
| 1957 | AACCATAGAACAATTTCAC | 1958 | GTGAAATTGTTCTATGGTT |
| 1959 | ACCATAGAACAATTTCACT | 1960 | AGTGAAATTGTTCTATGGT |
| 1961 | CCATAGAACAATTTCACTG | 1962 | CAGTGAAATTGTTCTATGG |
| 1963 | CATAGAACAATTTCACTGC | 1964 | GCAGTGAAATTGTTCTATG |
| 1965 | ATAGAACAATTTCACTGCA | 1966 | TGCAGTGAAATTGTTCTAT |
| 1967 | TAGAACAATTTCACTGCAA | 1968 | TTGCAGTGAAATTGTTCTA |
| 1969 | AGAACAATTTCACTGCAAA | 1970 | TTTGCAGTGAAATTGTTCT |
| 1971 | GAACAATTTCACTGCAAAC | 1972 | GTTTGCAGTGAAATTGTTC |
| 1973 | AACAATTTCACTGCAAACA | 1974 | TGTTTGCAGTGAAATTGTT |
| 1975 | ACAATTTCACTGCAAACAT | 1976 | ATGTTTGCAGTGAAATTGT |
| 1977 | CAATTTCACTGCAAACATG | 1978 | CATGTTTGCAGTGAAATTG |
| 1979 | AATTTCACTGCAAACATGC | 1980 | GCATGTTTGCAGTGAAATT |
| 1981 | ATTTCACTGCAAACATGCA | 1982 | TGCATGTTTGCAGTGAAAT |
| 1983 | TTTCACTGCAAACATGCAT | 1984 | ATGCATGTTTGCAGTGAAA |
| 1985 | TTCACTGCAAACATGCATG | 1986 | CATGCATGTTTGCAGTGAA |
| 1987 | TCACTGCAAACATGCATGA | 1988 | TCATGCATGTTTGCAGTGA |
| 1989 | CACTGCAAACATGCATGAT | 1990 | ATCATGCATGTTTGCAGTG |
| 1991 | ACTGCAAACATGCATGATT | 1992 | AATCATGCATGTTTGCAGT |
| 1993 | CTGCAAACATGCATGATTC | 1994 | GAATCATGCATGTTTGCAG |
| 1995 | TGCAAACATGCATGATTCT | 1996 | AGAATCATGCATGTTTGCA |
| 1997 | GCAAACATGCATGATTCTC | 1998 | GAGAATCATGCATGTTTGC |
| 1999 | CAAACATGCATGATTCTCC | 2000 | GGAGAATCATGCATGTTTG |
| 2001 | AAACATGCATGATTCTCCA | 2002 | TGGAGAATCATGCATGTTT |
| 2003 | AACATGCATGATTCTCCAA | 2004 | TTGGAGAATCATGCATGTT |
| 2005 | ACATGCATGATTCTCCAAG | 2006 | CTTGGAGAATCATGCATGT |
| 2007 | CATGCATGATTCTCCAAGA | 2008 | TCTTGGAGAATCATGCATG |
| 2009 | ATGCATGATTCTCCAAGAC | 2010 | GTCTTGGAGAATCATGCAT |
| 2011 | TGCATGATTCTCCAAGACA | 2012 | TGTCTTGGAGAATCATGCA |
| 2013 | GCATGATTCTCCAAGACAA | 2014 | TTGTCTTGGAGAATCATGC |
| 2015 | CATGATTCTCCAAGACAAA | 2016 | TTTGTCTTGGAGAATCATG |
| 2017 | ATGATTCTCCAAGACAAAA | 2018 | TTTTGTCTTGGAGAATCAT |
| 2019 | TGATTCTCCAAGACAAAAG | 2020 | CTTTTGTCTTGGAGAATCA |
| 2021 | GATTCTCCAAGACAAAAGA | 2022 | TCTTTTGTCTTGGAGAATC |
| 2023 | ATTCTCCAAGACAAAAGAA | 2024 | TTCTTTTGTCTTGGAGAAT |
| 2025 | TTCTCCAAGACAAAAGAAG | 2026 | CTTCTTTTGTCTTGGAGAA |
| 2027 | TCTCCAAGACAAAAGAAGA | 2028 | TCTTCTTTTGTCTTGGAGA |
| 2029 | CTCCAAGACAAAAGAAGAG | 2030 | CTCTTCTTTTGTCTTGGAG |
| 2031 | TCCAAGACAAAAGAAGAGA | 2032 | TCTCTTCTTTTGTCTTGGA |
| 2033 | CCAAGACAAAAGAAGAGAG | 2034 | CTCTCTTCTTTTGTCTTGG |
| 2035 | CAAGACAAAAGAAGAGAGA | 2036 | TCTCTCTTCTTTTGTCTTG |
| 2037 | AAGACAAAAGAAGAGAGAT | 2038 | ATCTCTCTTCTTTTGTCTT |
| 2039 | AGACAAAAGAAGAGAGATC | 2040 | GATCTCTCTTCTTTTGTCT |
| 2041 | GACAAAAGAAGAGAGATCC | 2042 | GGATCTCTCTTCTTTTGTC |
| 2043 | ACAAAAGAAGAGAGATCCT | 2044 | AGGATCTCTCTTCTTTTGT |
| 2045 | CAAAAGAAGAGAGATCCTA | 2046 | TAGGATCTCTCTTCTTTTG |
| 2047 | AAAAGAAGAGAGATCCTAA | 2048 | TTAGGATCTCTCTTCTTTT |
| 2049 | AAAGAAGAGAGATCCTAAA | 2050 | TTTAGGATCTCTCTTCTTT |
| 2051 | AAGAAGAGAGATCCTAAAG | 2052 | CTTTAGGATCTCTCTTCTT |
| 2053 | AGAAGAGAGATCCTAAAGG | 2054 | CCTTTAGGATCTCTCTTCT |
| 2055 | GAAGAGAGATCCTAAAGGC | 2056 | GCCTTTAGGATCTCTCTTC |
| 2057 | AAGAGAGATCCTAAAGGCA | 2058 | TGCCTTTAGGATCTCTCTT |
| 2059 | AGAGAGATCCTAAAGGCAA | 2060 | TTGCCTTTAGGATCTCTCT |
| 2061 | GAGAGATCCTAAAGGCAAT | 2062 | ATTGCCTTTAGGATCTCTC |
| 2063 | AGAGATCCTAAAGGCAATT | 2064 | AATTGCCTTTAGGATCTCT |
| 2065 | GAGATCCTAAAGGCAATTC | 2066 | GAATTGCCTTTAGGATCTC |
| 2067 | AGATCCTAAAGGCAATTCA | 2068 | TGAATTGCCTTTAGGATCT |
| 2069 | GATCCTAAAGGCAATTCAG | 2070 | CTGAATTGCCTTTAGGATC |
| 2071 | ATCCTAAAGGCAATTCAGA | 2072 | TCTGAATTGCCTTTAGGAT |
| 2073 | TCCTAAAGGCAATTCAGAT | 2074 | ATCTGAATTGCCTTTAGGA |
| 2075 | CCTAAAGGCAATTCAGATA | 2076 | TATCTGAATTGCCTTTAGG |
| 2077 | CTAAAGGCAATTCAGATAT | 2078 | ATATCTGAATTGCCTTTAG |
| 2079 | TAAAGGCAATTCAGATATC | 2080 | GATATCTGAATTGCCTTTA |
| 2081 | AAAGGCAATTCAGATATCC | 2082 | GGATATCTGAATTGCCTTT |
| 2083 | AAGGCAATTCAGATATCCC | 2084 | GGGATATCTGAATTGCCTT |
| 2085 | AGGCAATTCAGATATCCCC | 2086 | GGGGATATCTGAATTGCCT |
| 2087 | GGCAATTCAGATATCCCCA | 2088 | TGGGGATATCTGAATTGCC |
| 2089 | GCAATTCAGATATCCCCAA | 2090 | TTGGGGATATCTGAATTGC |
| 2091 | CAATTCAGATATCCCCAAG | 2092 | CTTGGGGATATCTGAATTG |
| 2093 | AATTCAGATATCCCCAAGG | 2094 | CCTTGGGGATATCTGAATT |
| 2095 | ATTCAGATATCCCCAAGGC | 2096 | GCCTTGGGGATATCTGAAT |
| 2097 | TTCAGATATCCCCAAGGCT | 2098 | AGCCTTGGGGATATCTGAA |
| 2099 | TCAGATATCCCCAAGGCTG | 2100 | CAGCCTTGGGGATATCTGA |
| 2101 | CAGATATCCCCAAGGCTGC | 2102 | GCAGCCTTGGGGATATCTG |
| 2103 | AGATATCCCCAAGGCTGCC | 2104 | GGCAGCCTTGGGGATATCT |
| 2105 | GATATCCCCAAGGCTGCCT | 2106 | AGGCAGCCTTGGGGATATC |
| 2107 | ATATCCCCAAGGCTGCCTC | 2108 | GAGGCAGCCTTGGGGATAT |
| 2109 | TATCCCCAAGGCTGCCTCT | 2110 | AGAGGCAGCCTTGGGGATA |
| 2111 | ATCCCCAAGGCTGCCTCTC | 2112 | GAGAGGCAGCCTTGGGGAT |
| 2113 | TCCCCAAGGCTGCCTCTCC | 2114 | GGAGAGGCAGCCTTGGGGA |
| 2115 | CCCCAAGGCTGCCTCTCCC | 2116 | GGGAGAGGCAGCCTTGGGG |
| 2117 | CCCAAGGCTGCCTCTCCCA | 2118 | TGGGAGAGGCAGCCTTGGG |
| 2119 | CCAAGGCTGCCTCTCCCAC | 2120 | GTGGGAGAGGCAGCCTTGG |
| 2121 | CAAGGCTGCCTCTCCCACC | 2122 | GGTGGGAGAGGCAGCCTTG |
| 2123 | AAGGCTGCCTCTCCCACCA | 2124 | TGGTGGGAGAGGCAGCCTT |
| 2125 | AGGCTGCCTCTCCCACCAC | 2126 | GTGGTGGGAGAGGCAGCCT |
| 2127 | GGCTGCCTCTCCCACCACA | 2128 | TGTGGTGGGAGAGGCAGCC |
| 2129 | GCTGCCTCTCCCACCACAA | 2130 | TTGTGGTGGGAGAGGCAGC |
| 2131 | CTGCCTCTCCCACCACAAG | 2132 | CTTGTGGTGGGAGAGGCAG |
| 2133 | TGCCTCTCCCACCACAAGC | 2134 | GCTTGTGGTGGGAGAGGCA |
| 2135 | GCCTCTCCCACCACAAGCC | 2136 | GGCTTGTGGTGGGAGAGGC |
| 2137 | CCTCTCCCACCACAAGCCC | 2138 | GGGCTTGTGGTGGGAGAGG |
| 2139 | CTCTCCCACCACAAGCCCA | 2140 | TGGGCTTGTGGTGGGAGAG |
| 2141 | TCTCCCACCACAAGCCCAG | 2142 | CTGGGCTTGTGGTGGGAGA |
| 2143 | CTCCCACCACAAGCCCAGA | 2144 | TCTGGGCTTGTGGTGGGAG |
| 2145 | TCCCACCACAAGCCCAGAG | 2146 | CTCTGGGCTTGTGGTGGGA |
| 2147 | CCCACCACAAGCCCAGAGT | 2148 | ACTCTGGGCTTGTGGTGGG |
| 2149 | CCACCACAAGCCCAGAGTG | 2150 | CACTCTGGGCTTGTGGTGG |
| 2151 | CACCACAAGCCCAGAGTGG | 2152 | CCACTCTGGGCTTGTGGTG |
| 2153 | ACCACAAGCCCAGAGTGGA | 2154 | TCCACTCTGGGCTTGTGGT |
| 2155 | CCACAAGCCCAGAGTGGAT | 2156 | ATCCACTCTGGGCTTGTGG |
| 2157 | CACAAGCCCAGAGTGGATG | 2158 | CATCCACTCTGGGCTTGTG |
| 2159 | ACAAGCCCAGAGTGGATGG | 2160 | CCATCCACTCTGGGCTTGT |
| 2161 | CAAGCCCAGAGTGGATGGG | 2162 | CCCATCCACTCTGGGCTTG |
| 2163 | AAGCCCAGAGTGGATGGGC | 2164 | GCCCATCCACTCTGGGCTT |
| 2165 | AGCCCAGAGTGGATGGGCT | 2166 | AGCCCATCCACTCTGGGCT |
| 2167 | GCCCAGAGTGGATGGGCTG | 2168 | CAGCCCATCCACTCTGGGC |
| 2169 | CCCAGAGTGGATGGGCTGG | 2170 | CCAGCCCATCCACTCTGGG |
| 2171 | CCAGAGTGGATGGGCTGGG | 2172 | CCCAGCCCATCCACTCTGG |
| 2173 | CAGAGTGGATGGGCTGGGG | 2174 | CCCCAGCCCATCCACTCTG |
| 2175 | AGAGTGGATGGGCTGGGGG | 2176 | CCCCCAGCCCATCCACTCT |
| 2177 | GAGTGGATGGGCTGGGGGA | 2178 | TCCCCCAGCCCATCCACTC |
| 2179 | AGTGGATGGGCTGGGGGAG | 2180 | CTCCCCCAGCCCATCCACT |
| 2181 | GTGGATGGGCTGGGGGAGG | 2182 | CCTCCCCCAGCCCATCCAC |
| 2183 | TGGATGGGCTGGGGGAGGG | 2184 | CCCTCCCCCAGCCCATCCA |
| 2185 | GGATGGGCTGGGGGAGGGG | 2186 | CCCCTCCCCCAGCCCATCC |
| 2187 | GATGGGCTGGGGGAGGGGT | 2188 | ACCCCTCCCCCAGCCCATC |
| 2189 | ATGGGCTGGGGGAGGGGTG | 2190 | CACCCCTCCCCCAGCCCAT |
| 2191 | TGGGCTGGGGGAGGGGTGC | 2192 | GCACCCCTCCCCCAGCCCA |
| 2193 | GGGCTGGGGGAGGGGTGCT | 2194 | AGCACCCCTCCCCCAGCCC |
| 2195 | GGCTGGGGGAGGGGTGCTG | 2196 | CAGCACCCCTCCCCCAGCC |
| 2197 | GCTGGGGGAGGGGTGCTGT | 2198 | ACAGCACCCCTCCCCCAGC |
| 2199 | CTGGGGGAGGGGTGCTGTT | 2200 | AACAGCACCCCTCCCCCAG |
| 2201 | TGGGGGAGGGGTGCTGTTT | 2202 | AAACAGCACCCCTCCCCCA |
| 2203 | GGGGGAGGGGTGCTGTTTT | 2204 | AAAACAGCACCCCTCCCCC |
| 2205 | GGGGAGGGGTGCTGTTTTA | 2206 | TAAAACAGCACCCCTCCCC |
| 2207 | GGGAGGGGTGCTGTTTTAA | 2208 | TTAAAACAGCACCCCTCCC |
| 2209 | GGAGGGGTGCTGTTTTAAT | 2210 | ATTAAAACAGCACCCCTCC |
| 2211 | GAGGGGTGCTGTTTTAATT | 2212 | AATTAAAACAGCACCCCTC |
| 2213 | AGGGGTGCTGTTTTAATTT | 2214 | AAATTAAAACAGCACCCCT |
| 2215 | GGGGTGCTGTTTTAATTTC | 2216 | GAAATTAAAACAGCACCCC |
| 2217 | GGGTGCTGTTTTAATTTCT | 2218 | AGAAATTAAAACAGCACCC |
| 2219 | GGTGCTGTTTTAATTTCTA | 2220 | TAGAAATTAAAACAGCACC |
| 2221 | GTGCTGTTTTAATTTCTAA | 2222 | TTAGAAATTAAAACAGCAC |
| 2223 | TGCTGTTTTAATTTCTAAA | 2224 | TTTAGAAATTAAAACAGCA |
| 2225 | GCTGTTTTAATTTCTAAAG | 2226 | CTTTAGAAATTAAAACAGC |
| 2227 | CTGTTTTAATTTCTAAAGG | 2228 | CCTTTAGAAATTAAAACAG |
| 2229 | TGTTTTAATTTCTAAAGGT | 2230 | ACCTTTAGAAATTAAAACA |
| 2231 | GTTTTAATTTCTAAAGGTA | 2232 | TACCTTTAGAAATTAAAAC |
| 2233 | TTTTAATTTCTAAAGGTAG | 2234 | CTACCTTTAGAAATTAAAA |
| 2235 | TTTAATTTCTAAAGGTAGG | 2236 | CCTACCTTTAGAAATTAAA |
| 2237 | TTAATTTCTAAAGGTAGGA | 2238 | TCCTACCTTTAGAAATTAA |
| 2239 | TAATTTCTAAAGGTAGGAC | 2240 | GTCCTACCTTTAGAAATTA |
| 2241 | AATTTCTAAAGGTAGGACC | 2242 | GGTCCTACCTTTAGAAATT |
| 2243 | ATTTCTAAAGGTAGGACCA | 2244 | TGGTCCTACCTTTAGAAAT |
| 2245 | TTTCTAAAGGTAGGACCAA | 2246 | TTGGTCCTACCTTTAGAAA |
| 2247 | TTCTAAAGGTAGGACCAAC | 2248 | GTTGGTCCTACCTTTAGAA |
| 2249 | TCTAAAGGTAGGACCAACA | 2250 | TGTTGGTCCTACCTTTAGA |
| 2251 | CTAAAGGTAGGACCAACAC | 2252 | GTGTTGGTCCTACCTTTAG |
| 2253 | TAAAGGTAGGACCAACACC | 2254 | GGTGTTGGTCCTACCTTTA |
| 2255 | AAAGGTAGGACCAACACCC | 2256 | GGGTGTTGGTCCTACCTTT |
| 2257 | AAGGTAGGACCAACACCCA | 2258 | TGGGTGTTGGTCCTACCTT |
| 2259 | AGGTAGGACCAACACCCAG | 2260 | CTGGGTGTTGGTCCTACCT |
| 2261 | GGTAGGACCAACACCCAGG | 2262 | CCTGGGTGTTGGTCCTACC |
| 2263 | GTAGGACCAACACCCAGGG | 2264 | CCCTGGGTGTTGGTCCTAC |
| 2265 | TAGGACCAACACCCAGGGG | 2266 | CCCCTGGGTGTTGGTCCTA |
| 2267 | AGGACCAACACCCAGGGGA | 2268 | TCCCCTGGGTGTTGGTCCT |
| 2269 | GGACCAACACCCAGGGGAT | 2270 | ATCCCCTGGGTGTTGGTCC |
| 2271 | GACCAACACCCAGGGGATC | 2272 | GATCCCCTGGGTGTTGGTC |
| 2273 | ACCAACACCCAGGGGATCA | 2274 | TGATCCCCTGGGTGTTGGT |
| 2275 | CCAACACCCAGGGGATCAG | 2276 | CTGATCCCCTGGGTGTTGG |
| 2277 | CAACACCCAGGGGATCAGT | 2278 | ACTGATCCCCTGGGTGTTG |
| 2279 | AACACCCAGGGGATCAGTG | 2280 | CACTGATCCCCTGGGTGTT |
| 2281 | ACACCCAGGGGATCAGTGA | 2282 | TCACTGATCCCCTGGGTGT |
| 2283 | CACCCAGGGGATCAGTGAA | 2284 | TTCACTGATCCCCTGGGTG |
| 2285 | ACCCAGGGGATCAGTGAAG | 2286 | CTTCACTGATCCCCTGGGT |
| 2287 | CCCAGGGGATCAGTGAAGG | 2288 | CCTTCACTGATCCCCTGGG |
| 2289 | CCAGGGGATCAGTGAAGGA | 2290 | TCCTTCACTGATCCCCTGG |
| 2291 | CAGGGGATCAGTGAAGGAA | 2292 | TTCCTTCACTGATCCCCTG |
| 2293 | AGGGGATCAGTGAAGGAAG | 2294 | CTTCCTTCACTGATCCCCT |
| 2295 | GGGGATCAGTGAAGGAAGA | 2296 | TCTTCCTTCACTGATCCCC |
| 2297 | GGGATCAGTGAAGGAAGAG | 2298 | CTCTTCCTTCACTGATCCC |
| 2299 | GGATCAGTGAAGGAAGAGA | 2300 | TCTCTTCCTTCACTGATCC |
| 2301 | GATCAGTGAAGGAAGAGAA | 2302 | TTCTCTTCCTTCACTGATC |
| 2303 | ATCAGTGAAGGAAGAGAAG | 2304 | CTTCTCTTCCTTCACTGAT |
| 2305 | TCAGTGAAGGAAGAGAAGG | 2306 | CCTTCTCTTCCTTCACTGA |
| 2307 | CAGTGAAGGAAGAGAAGGC | 2308 | GCCTTCTCTTCCTTCACTG |
| 2309 | AGTGAAGGAAGAGAAGGCC | 2310 | GGCCTTCTCTTCCTTCACT |
| 2311 | GTGAAGGAAGAGAAGGCCA | 2312 | TGGCCTTCTCTTCCTTCAC |
| 2313 | TGAAGGAAGAGAAGGCCAG | 2314 | CTGGCCTTCTCTTCCTTCA |
| 2315 | GAAGGAAGAGAAGGCCAGC | 2316 | GCTGGCCTTCTCTTCCTTC |
| 2317 | AAGGAAGAGAAGGCCAGCA | 2318 | TGCTGGCCTTCTCTTCCTT |
| 2319 | AGGAAGAGAAGGCCAGCAG | 2320 | CTGCTGGCCTTCTCTTCCT |
| 2321 | GGAAGAGAAGGCCAGCAGA | 2322 | TCTGCTGGCCTTCTCTTCC |
| 2323 | GAAGAGAAGGCCAGCAGAT | 2324 | ATCTGCTGGCCTTCTCTTC |
| 2325 | AAGAGAAGGCCAGCAGATC | 2326 | GATCTGCTGGCCTTCTCTT |
| 2327 | AGAGAAGGCCAGCAGATCA | 2328 | TGATCTGCTGGCCTTCTCT |
| 2329 | GAGAAGGCCAGCAGATCAC | 2330 | GTGATCTGCTGGCCTTCTC |
| 2331 | AGAAGGCCAGCAGATCACT | 2332 | AGTGATCTGCTGGCCTTCT |
| 2333 | GAAGGCCAGCAGATCACTG | 2334 | CAGTGATCTGCTGGCCTTC |
| 2335 | AAGGCCAGCAGATCACTGA | 2336 | TCAGTGATCTGCTGGCCTT |
| 2337 | AGGCCAGCAGATCACTGAG | 2338 | CTCAGTGATCTGCTGGCCT |
| 2339 | GGCCAGCAGATCACTGAGA | 2340 | TCTCAGTGATCTGCTGGCC |
| 2341 | GCCAGCAGATCACTGAGAG | 2342 | CTCTCAGTGATCTGCTGGC |
| 2343 | CCAGCAGATCACTGAGAGT | 2344 | ACTCTCAGTGATCTGCTGG |
| 2345 | CAGCAGATCACTGAGAGTG | 2346 | CACTCTCAGTGATCTGCTG |
| 2347 | AGCAGATCACTGAGAGTGC | 2348 | GCACTCTCAGTGATCTGCT |
| 2349 | GCAGATCACTGAGAGTGCA | 2350 | TGCACTCTCAGTGATCTGC |
| 2351 | CAGATCACTGAGAGTGCAA | 2352 | TTGCACTCTCAGTGATCTG |
| 2353 | AGATCACTGAGAGTGCAAC | 2354 | GTTGCACTCTCAGTGATCT |
| 2355 | GATCACTGAGAGTGCAACC | 2356 | GGTTGCACTCTCAGTGATC |
| 2357 | ATCACTGAGAGTGCAACCC | 2358 | GGGTTGCACTCTCAGTGAT |
| 2359 | TCACTGAGAGTGCAACCCC | 2360 | GGGGTTGCACTCTCAGTGA |
| 2361 | CACTGAGAGTGCAACCCCA | 2362 | TGGGGTTGCACTCTCAGTG |
| 2363 | ACTGAGAGTGCAACCCCAC | 2364 | GTGGGGTTGCACTCTCAGT |
| 2365 | CTGAGAGTGCAACCCCACC | 2366 | GGTGGGGTTGCACTCTCAG |
| 2367 | TGAGAGTGCAACCCCACCC | 2368 | GGGTGGGGTTGCACTCTCA |
| 2369 | GAGAGTGCAACCCCACCCT | 2370 | AGGGTGGGGTTGCACTCTC |
| 2371 | AGAGTGCAACCCCACCCTC | 2372 | GAGGGTGGGGTTGCACTCT |
| 2373 | GAGTGCAACCCCACCCTCC | 2374 | GGAGGGTGGGGTTGCACTC |
| 2375 | AGTGCAACCCCACCCTCCA | 2376 | TGGAGGGTGGGGTTGCACT |
| 2377 | GTGCAACCCCACCCTCCAC | 2378 | GTGGAGGGTGGGGTTGCAC |
| 2379 | TGCAACCCCACCCTCCACA | 2380 | TGTGGAGGGTGGGGTTGCA |
| 2381 | GCAACCCCACCCTCCACAG | 2382 | CTGTGGAGGGTGGGGTTGC |
| 2383 | CAACCCCACCCTCCACAGG | 2384 | CCTGTGGAGGGTGGGGTTG |
| 2385 | AACCCCACCCTCCACAGGA | 2386 | TCCTGTGGAGGGTGGGGTT |
| 2387 | ACCCCACCCTCCACAGGAA | 2388 | TTCCTGTGGAGGGTGGGGT |
| 2389 | CCCCACCCTCCACAGGAAA | 2390 | TTTCCTGTGGAGGGTGGGG |
| 2391 | CCCACCCTCCACAGGAAAT | 2392 | ATTTCCTGTGGAGGGTGGG |
| 2393 | CCACCCTCCACAGGAAATT | 2394 | AATTTCCTGTGGAGGGTGG |
| 2395 | CACCCTCCACAGGAAATTG | 2396 | CAATTTCCTGTGGAGGGTG |
| 2397 | ACCCTCCACAGGAAATTGC | 2398 | GCAATTTCCTGTGGAGGGT |
| 2399 | CCCTCCACAGGAAATTGCC | 2400 | GGCAATTTCCTGTGGAGGG |
| 2401 | CCTCCACAGGAAATTGCCT | 2402 | AGGCAATTTCCTGTGGAGG |
| 2403 | CTCCACAGGAAATTGCCTC | 2404 | GAGGCAATTTCCTGTGGAG |
| 2405 | TCCACAGGAAATTGCCTCA | 2406 | TGAGGCAATTTCCTGTGGA |
| 2407 | CCACAGGAAATTGCCTCAT | 2408 | ATGAGGCAATTTCCTGTGG |
| 2409 | CACAGGAAATTGCCTCATG | 2410 | CATGAGGCAATTTCCTGTG |
| 2411 | ACAGGAAATTGCCTCATGG | 2412 | CCATGAGGCAATTTCCTGT |
| 2413 | CAGGAAATTGCCTCATGGG | 2414 | CCCATGAGGCAATTTCCTG |
| 2415 | AGGAAATTGCCTCATGGGC | 2416 | GCCCATGAGGCAATTTCCT |
| 2417 | GGAAATTGCCTCATGGGCA | 2418 | TGCCCATGAGGCAATTTCC |
| 2419 | GAAATTGCCTCATGGGCAG | 2420 | CTGCCCATGAGGCAATTTC |
| 2421 | AAATTGCCTCATGGGCAGG | 2422 | CCTGCCCATGAGGCAATTT |
| 2423 | AATTGCCTCATGGGCAGGG | 2424 | CCCTGCCCATGAGGCAATT |
| 2425 | ATTGCCTCATGGGCAGGGC | 2426 | GCCCTGCCCATGAGGCAAT |
| 2427 | TTGCCTCATGGGCAGGGCC | 2428 | GGCCCTGCCCATGAGGCAA |
| 2429 | TGCCTCATGGGCAGGGCCA | 2430 | TGGCCCTGCCCATGAGGCA |
| 2431 | GCCTCATGGGCAGGGCCAC | 2432 | GTGGCCCTGCCCATGAGGC |
| 2433 | CCTCATGGGCAGGGCCACA | 2434 | TGTGGCCCTGCCCATGAGG |
| 2435 | CTCATGGGCAGGGCCACAG | 2436 | CTGTGGCCCTGCCCATGAG |
| 2437 | TCATGGGCAGGGCCACAGC | 2438 | GCTGTGGCCCTGCCCATGA |
| 2439 | CATGGGCAGGGCCACAGCA | 2440 | TGCTGTGGCCCTGCCCATG |
| 2441 | ATGGGCAGGGCCACAGCAG | 2442 | CTGCTGTGGCCCTGCCCAT |
| 2443 | TGGGCAGGGCCACAGCAGA | 2444 | TCTGCTGTGGCCCTGCCCA |
| 2445 | GGGCAGGGCCACAGCAGAG | 2446 | CTCTGCTGTGGCCCTGCCC |
| 2447 | GGCAGGGCCACAGCAGAGA | 2448 | TCTCTGCTGTGGCCCTGCC |
| 2449 | GCAGGGCCACAGCAGAGAG | 2450 | CTCTCTGCTGTGGCCCTGC |
| 2451 | CAGGGCCACAGCAGAGAGA | 2452 | TCTCTCTGCTGTGGCCCTG |
| 2453 | AGGGCCACAGCAGAGAGAC | 2454 | GTCTCTCTGCTGTGGCCCT |
| 2455 | GGGCCACAGCAGAGAGACA | 2456 | TGTCTCTCTGCTGTGGCCC |
| 2457 | GGCCACAGCAGAGAGACAC | 2458 | GTGTCTCTCTGCTGTGGCC |
| 2459 | GCCACAGCAGAGAGACACA | 2460 | TGTGTCTCTCTGCTGTGGC |
| 2461 | CCACAGCAGAGAGACACAG | 2462 | CTGTGTCTCTCTGCTGTGG |
| 2463 | CACAGCAGAGAGACACAGC | 2464 | GCTGTGTCTCTCTGCTGTG |
| 2465 | ACAGCAGAGAGACACAGCA | 2466 | TGCTGTGTCTCTCTGCTGT |
| 2467 | CAGCAGAGAGACACAGCAT | 2468 | ATGCTGTGTCTCTCTGCTG |
| 2469 | AGCAGAGAGACACAGCATG | 2470 | CATGCTGTGTCTCTCTGCT |
| 2471 | GCAGAGAGACACAGCATGG | 2472 | CCATGCTGTGTCTCTCTGC |
| 2473 | CAGAGAGACACAGCATGGG | 2474 | CCCATGCTGTGTCTCTCTG |
| 2475 | AGAGAGACACAGCATGGGC | 2476 | GCCCATGCTGTGTCTCTCT |
| 2477 | GAGAGACACAGCATGGGCA | 2478 | TGCCCATGCTGTGTCTCTC |
| 2479 | AGAGACACAGCATGGGCAG | 2480 | CTGCCCATGCTGTGTCTCT |
| 2481 | GAGACACAGCATGGGCAGT | 2482 | ACTGCCCATGCTGTGTCTC |
| 2483 | AGACACAGCATGGGCAGTG | 2484 | CACTGCCCATGCTGTGTCT |
| 2485 | GACACAGCATGGGCAGTGC | 2486 | GCACTGCCCATGCTGTGTC |
| 2487 | ACACAGCATGGGCAGTGCC | 2488 | GGCACTGCCCATGCTGTGT |
| 2489 | CACAGCATGGGCAGTGCCT | 2490 | AGGCACTGCCCATGCTGTG |
| 2491 | ACAGCATGGGCAGTGCCTT | 2492 | AAGGCACTGCCCATGCTGT |
| 2493 | CAGCATGGGCAGTGCCTTC | 2494 | GAAGGCACTGCCCATGCTG |
| 2495 | AGCATGGGCAGTGCCTTCC | 2496 | GGAAGGCACTGCCCATGCT |
| 2497 | GCATGGGCAGTGCCTTCCC | 2498 | GGGAAGGCACTGCCCATGC |
| 2499 | CATGGGCAGTGCCTTCCCT | 2500 | AGGGAAGGCACTGCCCATG |
| 2501 | ATGGGCAGTGCCTTCCCTG | 2502 | CAGGGAAGGCACTGCCCAT |
| 2503 | TGGGCAGTGCCTTCCCTGC | 2504 | GCAGGGAAGGCACTGCCCA |
| 2505 | GGGCAGTGCCTTCCCTGCC | 2506 | GGCAGGGAAGGCACTGCCC |
| 2507 | GGCAGTGCCTTCCCTGCCT | 2508 | AGGCAGGGAAGGCACTGCC |
| 2509 | GCAGTGCCTTCCCTGCCTG | 2510 | CAGGCAGGGAAGGCACTGC |
| 2511 | CAGTGCCTTCCCTGCCTGT | 2512 | ACAGGCAGGGAAGGCACTG |
| 2513 | AGTGCCTTCCCTGCCTGTG | 2514 | CACAGGCAGGGAAGGCACT |
| 2515 | GTGCCTTCCCTGCCTGTGG | 2516 | CCACAGGCAGGGAAGGCAC |
| 2517 | TGCCTTCCCTGCCTGTGGG | 2518 | CCCACAGGCAGGGAAGGCA |
| 2519 | GCCTTCCCTGCCTGTGGGG | 2520 | CCCCACAGGCAGGGAAGGC |
| 2521 | CCTTCCCTGCCTGTGGGGG | 2522 | CCCCCACAGGCAGGGAAGG |
| 2523 | CTTCCCTGCCTGTGGGGGT | 2524 | ACCCCCACAGGCAGGGAAG |
| 2525 | TTCCCTGCCTGTGGGGGTC | 2526 | GACCCCCACAGGCAGGGAA |
| 2527 | TCCCTGCCTGTGGGGGTCA | 2528 | TGACCCCCACAGGCAGGGA |
| 2529 | CCCTGCCTGTGGGGGTCAT | 2530 | ATGACCCCCACAGGCAGGG |
| 2531 | CCTGCCTGTGGGGGTCATG | 2532 | CATGACCCCCACAGGCAGG |
| 2533 | CTGCCTGTGGGGGTCATGC | 2534 | GCATGACCCCCACAGGCAG |
| 2535 | TGCCTGTGGGGGTCATGCT | 2536 | AGCATGACCCCCACAGGCA |
| 2537 | GCCTGTGGGGGTCATGCTG | 2538 | CAGCATGACCCCCACAGGC |
| 2539 | CCTGTGGGGGTCATGCTGC | 2540 | GCAGCATGACCCCCACAGG |
| 2541 | CTGTGGGGGTCATGCTGCC | 2542 | GGCAGCATGACCCCCACAG |
| 2543 | TGTGGGGGTCATGCTGCCA | 2544 | TGGCAGCATGACCCCCACA |
| 2545 | GTGGGGGTCATGCTGCCAC | 2546 | GTGGCAGCATGACCCCCAC |
| 2547 | TGGGGGTCATGCTGCCACT | 2548 | AGTGGCAGCATGACCCCCA |
| 2549 | GGGGGTCATGCTGCCACTT | 2550 | AAGTGGCAGCATGACCCCC |
| 2551 | GGGGTCATGCTGCCACTTT | 2552 | AAAGTGGCAGCATGACCCC |
| 2553 | GGGTCATGCTGCCACTTTT | 2554 | AAAAGTGGCAGCATGACCC |
| 2555 | GGTCATGCTGCCACTTTTA | 2556 | TAAAAGTGGCAGCATGACC |
| 2557 | GTCATGCTGCCACTTTTAA | 2558 | TTAAAAGTGGCAGCATGAC |
| 2559 | TCATGCTGCCACTTTTAAT | 2560 | ATTAAAAGTGGCAGCATGA |
| 2561 | CATGCTGCCACTTTTAATG | 2562 | CATTAAAAGTGGCAGCATG |
| 2563 | ATGCTGCCACTTTTAATGG | 2564 | CCATTAAAAGTGGCAGCAT |
| 2565 | TGCTGCCACTTTTAATGGG | 2566 | CCCATTAAAAGTGGCAGCA |
| 2567 | GCTGCCACTTTTAATGGGT | 2568 | ACCCATTAAAAGTGGCAGC |
| 2569 | CTGCCACTTTTAATGGGTC | 2570 | GACCCATTAAAAGTGGCAG |
| 2571 | TGCCACTTTTAATGGGTCC | 2572 | GGACCCATTAAAAGTGGCA |
| 2573 | GCCACTTTTAATGGGTCCT | 2574 | AGGACCCATTAAAAGTGGC |
| 2575 | CCACTTTTAATGGGTCCTC | 2576 | GAGGACCCATTAAAAGTGG |
| 2577 | CACTTTTAATGGGTCCTCC | 2578 | GGAGGACCCATTAAAAGTG |
| 2579 | ACTTTTAATGGGTCCTCCA | 2580 | TGGAGGACCCATTAAAAGT |
| 2581 | CTTTTAATGGGTCCTCCAC | 2582 | GTGGAGGACCCATTAAAAG |
| 2583 | TTTTAATGGGTCCTCCACC | 2584 | GGTGGAGGACCCATTAAAA |
| 2585 | TTTAATGGGTCCTCCACCC | 2586 | GGGTGGAGGACCCATTAAA |
| 2587 | TTAATGGGTCCTCCACCCA | 2588 | TGGGTGGAGGACCCATTAA |
| 2589 | TAATGGGTCCTCCACCCAA | 2590 | TTGGGTGGAGGACCCATTA |
| 2591 | AATGGGTCCTCCACCCAAC | 2592 | GTTGGGTGGAGGACCCATT |
| 2593 | ATGGGTCCTCCACCCAACG | 2594 | CGTTGGGTGGAGGACCCAT |
| 2595 | TGGGTCCTCCACCCAACGG | 2596 | CCGTTGGGTGGAGGACCCA |
| 2597 | GGGTCCTCCACCCAACGGG | 2598 | CCCGTTGGGTGGAGGACCC |
| 2599 | GGTCCTCCACCCAACGGGG | 2600 | CCCCGTTGGGTGGAGGACC |
| 2601 | GTCCTCCACCCAACGGGGT | 2602 | ACCCCGTTGGGTGGAGGAC |
| 2603 | TCCTCCACCCAACGGGGTC | 2604 | GACCCCGTTGGGTGGAGGA |
| 2605 | CCTCCACCCAACGGGGTCA | 2606 | TGACCCCGTTGGGTGGAGG |
| 2607 | CTCCACCCAACGGGGTCAG | 2608 | CTGACCCCGTTGGGTGGAG |
| 2609 | TCCACCCAACGGGGTCAGG | 2610 | CCTGACCCCGTTGGGTGGA |
| 2611 | CCACCCAACGGGGTCAGGG | 2612 | CCCTGACCCCGTTGGGTGG |
| 2613 | CACCCAACGGGGTCAGGGA | 2614 | TCCCTGACCCCGTTGGGTG |
| 2615 | ACCCAACGGGGTCAGGGAG | 2616 | CTCCCTGACCCCGTTGGGT |
| 2617 | CCCAACGGGGTCAGGGAGG | 2618 | CCTCCCTGACCCCGTTGGG |
| 2619 | CCAACGGGGTCAGGGAGGT | 2620 | ACCTCCCTGACCCCGTTGG |
| 2621 | CAACGGGGTCAGGGAGGTG | 2622 | CACCTCCCTGACCCCGTTG |
| 2623 | AACGGGGTCAGGGAGGTGG | 2624 | CCACCTCCCTGACCCCGTT |
| 2625 | ACGGGGTCAGGGAGGTGGT | 2626 | ACCACCTCCCTGACCCCGT |
| 2627 | CGGGGTCAGGGAGGTGGTG | 2628 | CACCACCTCCCTGACCCCG |
| 2629 | GGGGTCAGGGAGGTGGTGC | 2630 | GCACCACCTCCCTGACCCC |
| 2631 | GGGTCAGGGAGGTGGTGCT | 2632 | AGCACCACCTCCCTGACCC |
| 2633 | GGTCAGGGAGGTGGTGCTG | 2634 | CAGCACCACCTCCCTGACC |
| 2635 | GTCAGGGAGGTGGTGCTGC | 2636 | GCAGCACCACCTCCCTGAC |
| 2637 | TCAGGGAGGTGGTGCTGCC | 2638 | GGCAGCACCACCTCCCTGA |
| 2639 | CAGGGAGGTGGTGCTGCCC | 2640 | GGGCAGCACCACCTCCCTG |
| 2641 | AGGGAGGTGGTGCTGCCCC | 2642 | GGGGCAGCACCACCTCCCT |
| 2643 | GGGAGGTGGTGCTGCCCCA | 2644 | TGGGGCAGCACCACCTCCC |
| 2645 | GGAGGTGGTGCTGCCCCAG | 2646 | CTGGGGCAGCACCACCTCC |
| 2647 | GAGGTGGTGCTGCCCCAGT | 2648 | ACTGGGGCAGCACCACCTC |
| 2649 | AGGTGGTGCTGCCCCAGTG | 2650 | CACTGGGGCAGCACCACCT |
| 2651 | GGTGGTGCTGCCCCAGTGG | 2652 | CCACTGGGGCAGCACCACC |
| 2653 | GTGGTGCTGCCCCAGTGGG | 2654 | CCCACTGGGGCAGCACCAC |
| 2655 | TGGTGCTGCCCCAGTGGGC | 2656 | GCCCACTGGGGCAGCACCA |
| 2657 | GGTGCTGCCCCAGTGGGCC | 2658 | GGCCCACTGGGGCAGCACC |
| 2659 | GTGCTGCCCCAGTGGGCCA | 2660 | TGGCCCACTGGGGCAGCAC |
| 2661 | TGCTGCCCCAGTGGGCCAT | 2662 | ATGGCCCACTGGGGCAGCA |
| 2663 | GCTGCCCCAGTGGGCCATG | 2664 | CATGGCCCACTGGGGCAGC |
| 2665 | CTGCCCCAGTGGGCCATGA | 2666 | TCATGGCCCACTGGGGCAG |
| 2667 | TGCCCCAGTGGGCCATGAT | 2668 | ATCATGGCCCACTGGGGCA |
| 2669 | GCCCCAGTGGGCCATGATT | 2670 | AATCATGGCCCACTGGGGC |
| 2671 | CCCCAGTGGGCCATGATTA | 2672 | TAATCATGGCCCACTGGGG |
| 2673 | CCCAGTGGGCCATGATTAT | 2674 | ATAATCATGGCCCACTGGG |
| 2675 | CCAGTGGGCCATGATTATC | 2676 | GATAATCATGGCCCACTGG |
| 2677 | CAGTGGGCCATGATTATCT | 2678 | AGATAATCATGGCCCACTG |
| 2679 | AGTGGGCCATGATTATCTT | 2680 | AAGATAATCATGGCCCACT |
| 2681 | GTGGGCCATGATTATCTTA | 2682 | TAAGATAATCATGGCCCAC |
| 2683 | TGGGCCATGATTATCTTAA | 2684 | TTAAGATAATCATGGCCCA |
| 2685 | GGGCCATGATTATCTTAAA | 2686 | TTTAAGATAATCATGGCCC |
| 2687 | GGCCATGATTATCTTAAAG | 2688 | CTTTAAGATAATCATGGCC |
| 2689 | GCCATGATTATCTTAAAGG | 2690 | CCTTTAAGATAATCATGGC |
| 2691 | CCATGATTATCTTAAAGGC | 2692 | GCCTTTAAGATAATCATGG |
| 2693 | CATGATTATCTTAAAGGCA | 2694 | TGCCTTTAAGATAATCATG |
| 2695 | ATGATTATCTTAAAGGCAT | 2696 | ATGCCTTTAAGATAATCAT |
| 2697 | TGATTATCTTAAAGGCATT | 2698 | AATGCCTTTAAGATAATCA |
| 2699 | GATTATCTTAAAGGCATTA | 2700 | TAATGCCTTTAAGATAATC |
| 2701 | ATTATCTTAAAGGCATTAT | 2702 | ATAATGCCTTTAAGATAAT |
| 2703 | TTATCTTAAAGGCATTATT | 2704 | AATAATGCCTTTAAGATAA |
| 2705 | TATCTTAAAGGCATTATTC | 2706 | GAATAATGCCTTTAAGATA |
| 2707 | ATCTTAAAGGCATTATTCT | 2708 | AGAATAATGCCTTTAAGAT |
| 2709 | TCTTAAAGGCATTATTCTC | 2710 | GAGAATAATGCCTTTAAGA |
| 2711 | CTTAAAGGCATTATTCTCC | 2712 | GGAGAATAATGCCTTTAAG |
| 2713 | TTAAAGGCATTATTCTCCA | 2714 | TGGAGAATAATGCCTTTAA |
| 2715 | TAAAGGCATTATTCTCCAG | 2716 | CTGGAGAATAATGCCTTTA |
| 2717 | AAAGGCATTATTCTCCAGC | 2718 | GCTGGAGAATAATGCCTTT |
| 2719 | AAGGCATTATTCTCCAGCC | 2720 | GGCTGGAGAATAATGCCTT |
| 2721 | AGGCATTATTCTCCAGCCT | 2722 | AGGCTGGAGAATAATGCCT |
| 2723 | GGCATTATTCTCCAGCCTT | 2724 | AAGGCTGGAGAATAATGCC |
| 2725 | GCATTATTCTCCAGCCTTA | 2726 | TAAGGCTGGAGAATAATGC |
| 2727 | CATTATTCTCCAGCCTTAA | 2728 | TTAAGGCTGGAGAATAATG |
| 2729 | ATTATTCTCCAGCCTTAAG | 2730 | CTTAAGGCTGGAGAATAAT |
| 2731 | TTATTCTCCAGCCTTAAGT | 2732 | ACTTAAGGCTGGAGAATAA |
| 2733 | TATTCTCCAGCCTTAAGTA | 2734 | TACTTAAGGCTGGAGAATA |
| 2735 | ATTCTCCAGCCTTAAGTAA | 2736 | TTACTTAAGGCTGGAGAAT |
| 2737 | TTCTCCAGCCTTAAGTAAG | 2738 | CTTACTTAAGGCTGGAGAA |
| 2739 | TCTCCAGCCTTAAGTAAGA | 2740 | TCTTACTTAAGGCTGGAGA |
| 2741 | CTCCAGCCTTAAGTAAGAT | 2742 | ATCTTACTTAAGGCTGGAG |
| 2743 | TCCAGCCTTAAGTAAGATC | 2744 | GATCTTACTTAAGGCTGGA |
| 2745 | CCAGCCTTAAGTAAGATCT | 2746 | AGATCTTACTTAAGGCTGG |
| 2747 | CAGCCTTAAGTAAGATCTT | 2748 | AAGATCTTACTTAAGGCTG |
| 2749 | AGCCTTAAGTAAGATCTTA | 2750 | TAAGATCTTACTTAAGGCT |
| 2751 | GCCTTAAGTAAGATCTTAG | 2752 | CTAAGATCTTACTTAAGGC |
| 2753 | CCTTAAGTAAGATCTTAGG | 2754 | CCTAAGATCTTACTTAAGG |
| 2755 | CTTAAGTAAGATCTTAGGA | 2756 | TCCTAAGATCTTACTTAAG |
| 2757 | TTAAGTAAGATCTTAGGAC | 2758 | GTCCTAAGATCTTACTTAA |
| 2759 | TAAGTAAGATCTTAGGACG | 2760 | CGTCCTAAGATCTTACTTA |
| 2761 | AAGTAAGATCTTAGGACGT | 2762 | ACGTCCTAAGATCTTACTT |
| 2763 | AGTAAGATCTTAGGACGTT | 2764 | AACGTCCTAAGATCTTACT |
| 2765 | GTAAGATCTTAGGACGTTT | 2766 | AAACGTCCTAAGATCTTAC |
| 2767 | TAAGATCTTAGGACGTTTC | 2768 | GAAACGTCCTAAGATCTTA |
| 2769 | AAGATCTTAGGACGTTTCC | 2770 | GGAAACGTCCTAAGATCTT |
| 2771 | AGATCTTAGGACGTTTCCT | 2772 | AGGAAACGTCCTAAGATCT |
| 2773 | GATCTTAGGACGTTTCCTT | 2774 | AAGGAAACGTCCTAAGATC |
| 2775 | ATCTTAGGACGTTTCCTTT | 2776 | AAAGGAAACGTCCTAAGAT |
| 2777 | TCTTAGGACGTTTCCTTTG | 2778 | CAAAGGAAACGTCCTAAGA |
| 2779 | CTTAGGACGTTTCCTTTGC | 2780 | GCAAAGGAAACGTCCTAAG |
| 2781 | TTAGGACGTTTCCTTTGCT | 2782 | AGCAAAGGAAACGTCCTAA |
| 2783 | TAGGACGTTTCCTTTGCTA | 2784 | TAGCAAAGGAAACGTCCTA |
| 2785 | AGGACGTTTCCTTTGCTAT | 2786 | ATAGCAAAGGAAACGTCCT |
| 2787 | GGACGTTTCCTTTGCTATG | 2788 | CATAGCAAAGGAAACGTCC |
| 2789 | GACGTTTCCTTTGCTATGA | 2790 | TCATAGCAAAGGAAACGTC |
| 2791 | ACGTTTCCTTTGCTATGAT | 2792 | ATCATAGCAAAGGAAACGT |
| 2793 | CGTTTCCTTTGCTATGATT | 2794 | AATCATAGCAAAGGAAACG |
| 2795 | GTTTCCTTTGCTATGATTT | 2796 | AAATCATAGCAAAGGAAAC |
| 2797 | TTTCCTTTGCTATGATTTG | 2798 | CAAATCATAGCAAAGGAAA |
| 2799 | TTCCTTTGCTATGATTTGT | 2800 | ACAAATCATAGCAAAGGAA |
| 2801 | TCCTTTGCTATGATTTGTA | 2802 | TACAAATCATAGCAAAGGA |
| 2803 | CCTTTGCTATGATTTGTAC | 2804 | GTACAAATCATAGCAAAGG |
| 2805 | CTTTGCTATGATTTGTACT | 2806 | AGTACAAATCATAGCAAAG |
| 2807 | TTTGCTATGATTTGTACTT | 2808 | AAGTACAAATCATAGCAAA |
| 2809 | TTGCTATGATTTGTACTTG | 2810 | CAAGTACAAATCATAGCAA |
| 2811 | TGCTATGATTTGTACTTGC | 2812 | GCAAGTACAAATCATAGCA |
| 2813 | GCTATGATTTGTACTTGCT | 2814 | AGCAAGTACAAATCATAGC |
| 2815 | CTATGATTTGTACTTGCTT | 2816 | AAGCAAGTACAAATCATAG |
| 2817 | TATGATTTGTACTTGCTTG | 2818 | CAAGCAAGTACAAATCATA |
| 2819 | ATGATTTGTACTTGCTTGA | 2820 | TCAAGCAAGTACAAATCAT |
| 2821 | TGATTTGTACTTGCTTGAG | 2822 | CTCAAGCAAGTACAAATCA |
| 2823 | GATTTGTACTTGCTTGAGT | 2824 | ACTCAAGCAAGTACAAATC |
| 2825 | ATTTGTACTTGCTTGAGTC | 2826 | GACTCAAGCAAGTACAAAT |
| 2827 | TTTGTACTTGCTTGAGTCC | 2828 | GGACTCAAGCAAGTACAAA |
| 2829 | TTGTACTTGCTTGAGTCCC | 2830 | GGGACTCAAGCAAGTACAA |
| 2831 | TGTACTTGCTTGAGTCCCA | 2832 | TGGGACTCAAGCAAGTACA |
| 2833 | GTACTTGCTTGAGTCCCAT | 2834 | ATGGGACTCAAGCAAGTAC |
| 2835 | TACTTGCTTGAGTCCCATG | 2836 | CATGGGACTCAAGCAAGTA |
| 2837 | ACTTGCTTGAGTCCCATGA | 2838 | TCATGGGACTCAAGCAAGT |
| 2839 | CTTGCTTGAGTCCCATGAC | 2840 | GTCATGGGACTCAAGCAAG |
| 2841 | TTGCTTGAGTCCCATGACT | 2842 | AGTCATGGGACTCAAGCAA |
| 2843 | TGCTTGAGTCCCATGACTG | 2844 | CAGTCATGGGACTCAAGCA |
| 2845 | GCTTGAGTCCCATGACTGT | 2846 | ACAGTCATGGGACTCAAGC |
| 2847 | CTTGAGTCCCATGACTGTT | 2848 | AACAGTCATGGGACTCAAG |
| 2849 | TTGAGTCCCATGACTGTTT | 2850 | AAACAGTCATGGGACTCAA |
| 2851 | TGAGTCCCATGACTGTTTC | 2852 | GAAACAGTCATGGGACTCA |
| 2853 | GAGTCCCATGACTGTTTCT | 2854 | AGAAACAGTCATGGGACTC |
| 2855 | AGTCCCATGACTGTTTCTC | 2856 | GAGAAACAGTCATGGGACT |
| 2857 | GTCCCATGACTGTTTCTCT | 2858 | AGAGAAACAGTCATGGGAC |
| 2859 | TCCCATGACTGTTTCTCTT | 2860 | AAGAGAAACAGTCATGGGA |
| 2861 | CCCATGACTGTTTCTCTTC | 2862 | GAAGAGAAACAGTCATGGG |
| 2863 | CCATGACTGTTTCTCTTCC | 2864 | GGAAGAGAAACAGTCATGG |
| 2865 | CATGACTGTTTCTCTTCCT | 2866 | AGGAAGAGAAACAGTCATG |
| 2867 | ATGACTGTTTCTCTTCCTC | 2868 | GAGGAAGAGAAACAGTCAT |
| 2869 | TGACTGTTTCTCTTCCTCT | 2870 | AGAGGAAGAGAAACAGTCA |
| 2871 | GACTGTTTCTCTTCCTCTC | 2872 | GAGAGGAAGAGAAACAGTC |
| 2873 | ACTGTTTCTCTTCCTCTCT | 2874 | AGAGAGGAAGAGAAACAGT |
| 2875 | CTGTTTCTCTTCCTCTCTT | 2876 | AAGAGAGGAAGAGAAACAG |
| 2877 | TGTTTCTCTTCCTCTCTTT | 2878 | AAAGAGAGGAAGAGAAACA |
| 2879 | GTTTCTCTTCCTCTCTTTC | 2880 | GAAAGAGAGGAAGAGAAAC |
| 2881 | TTTCTCTTCCTCTCTTTCT | 2882 | AGAAAGAGAGGAAGAGAAA |
| 2883 | TTCTCTTCCTCTCTTTCTT | 2884 | AAGAAAGAGAGGAAGAGAA |
| 2885 | TCTCTTCCTCTCTTTCTTC | 2886 | GAAGAAAGAGAGGAAGAGA |
| 2887 | CTCTTCCTCTCTTTCTTCC | 2888 | GGAAGAAAGAGAGGAAGAG |
| 2889 | TCTTCCTCTCTTTCTTCCT | 2890 | AGGAAGAAAGAGAGGAAGA |
| 2891 | CTTCCTCTCTTTCTTCCTT | 2892 | AAGGAAGAAAGAGAGGAAG |
| 2893 | TTCCTCTCTTTCTTCCTTT | 2894 | AAAGGAAGAAAGAGAGGAA |
| 2895 | TCCTCTCTTTCTTCCTTTT | 2896 | AAAAGGAAGAAAGAGAGGA |
| 2897 | CCTCTCTTTCTTCCTTTTG | 2898 | CAAAAGGAAGAAAGAGAGG |
| 2899 | CTCTCTTTCTTCCTTTTGG | 2900 | CCAAAAGGAAGAAAGAGAG |
| 2901 | TCTCTTTCTTCCTTTTGGA | 2902 | TCCAAAAGGAAGAAAGAGA |
| 2903 | CTCTTTCTTCCTTTTGGAA | 2904 | TTCCAAAAGGAAGAAAGAG |
| 2905 | TCTTTCTTCCTTTTGGAAT | 2906 | ATTCCAAAAGGAAGAAAGA |
| 2907 | CTTTCTTCCTTTTGGAATA | 2908 | TATTCCAAAAGGAAGAAAG |
| 2909 | TTTCTTCCTTTTGGAATAG | 2910 | CTATTCCAAAAGGAAGAAA |
| 2911 | TTCTTCCTTTTGGAATAGT | 2912 | ACTATTCCAAAAGGAAGAA |
| 2913 | TCTTCCTITTGGAATAGTA | 2914 | TACTATTCCAAAAGGAAGA |
| 2915 | CTTCCTTTTGGAATAGTAA | 2916 | TTACTATTCCAAAAGGAAG |
| 2917 | TTCCTTTTGGAATAGTAAT | 2918 | ATTACTATTCCAAAAGGAA |
| 2919 | TCCTTTTGGAATAGTAATA | 2920 | TATTACTATTCCAAAAGGA |
| 2921 | CCTTTTGGAATAGTAATAT | 2922 | ATATTACTATTCCAAAAGG |
| 2923 | CTTTTGGAATAGTAATATC | 2924 | GATATTACTATTCCAAAAG |
| 2925 | TTTTGGAATAGTAATATCC | 2926 | GGATATTACTATTCCAAAA |
| 2927 | TTTGGAATAGTAATATCCA | 2928 | TGGATATTACTATTCCAAA |
| 2929 | TTGGAATAGTAATATCCAT | 2930 | ATGGATATTACTATTCCAA |
| 2931 | TGGAATAGTAATATCCATC | 2932 | GATGGATATTACTATTCCA |
| 2933 | GGAATAGTAATATCCATCC | 2934 | GGATGGATATTACTATTCC |
| 2935 | GAATAGTAATATCCATCCT | 2936 | AGGATGGATATTACTATTC |
| 2937 | AATAGTAATATCCATCCTA | 2938 | TAGGATGGATATTACTATT |
| 2939 | ATAGTAATATCCATCCTAT | 2940 | ATAGGATGGATATTACTAT |
| 2941 | TAGTAATATCCATCCTATG | 2942 | CATAGGATGGATATTACTA |
| 2943 | AGTAATATCCATCCTATGT | 2944 | ACATAGGATGGATATTACT |
| 2945 | GTAATATCCATCCTATGTT | 2946 | AACATAGGATGGATATTAC |
| 2947 | TAATATCCATCCTATGTTT | 2948 | AAACATAGGATGGATATTA |
| 2949 | AATATCCATCCTATGTTTG | 2950 | CAAACATAGGATGGATATT |
| 2951 | ATATCCATCCTATGTTTGT | 2952 | ACAAACATAGGATGGATAT |
| 2953 | TATCCATCCTATGTTTGTC | 2954 | GACAAACATAGGATGGATA |
| 2955 | ATCCATCCTATGTTTGTCC | 2956 | GGACAAACATAGGATGGAT |
| 2957 | TCCATCCTATGTTTGTCCC | 2958 | GGGACAAACATAGGATGGA |
| 2959 | CCATCCTATGTTTGTCCCA | 2960 | TGGGACAAACATAGGATGG |
| 2961 | CATCCTATGTTTGTCCCAC | 2962 | GTGGGACAAACATAGGATG |
| 2963 | ATCCTATGTTTGTCCCACT | 2964 | AGTGGGACAAACATAGGAT |
| 2965 | TCCTATGTTTGTCCCACTA | 2966 | TAGTGGGACAAACATAGGA |
| 2967 | CCTATGTTTGTCCCACTAT | 2968 | ATAGTGGGACAAACATAGG |
| 2969 | CTATGTTTGTCCCACTATT | 2970 | AATAGTGGGACAAACATAG |
| 2971 | TATGTTTGTCCCACTATTG | 2972 | CAATAGTGGGACAAACATA |
| 2973 | ATGTTTGTCCCACTATTGT | 2974 | ACAATAGTGGGACAAACAT |
| 2975 | TGTTTGTCCCACTATTGTA | 2976 | TACAATAGTGGGACAAACA |
| 2977 | GTTTGTCCCACTATTGTAT | 2978 | ATACAATAGTGGGACAAAC |
| 2979 | TTTGTCCCACTATTGTATT | 2980 | AATACAATAGTGGGACAAA |
| 2981 | TTGTCCCACTATTGTATTT | 2982 | AAATACAATAGTGGGACAA |
| 2983 | TGTCCCACTATTGTATTTT | 2984 | AAAATACAATAGTGGGACA |
| 2985 | GTCCCACTATTGTATTTTG | 2986 | CAAAATACAATAGTGGGAC |
| 2987 | TCCCACTATTGTATTTTGG | 2988 | CCAAAATACAATAGTGGGA |
| 2989 | CCCACTATTGTATTTTGGA | 2990 | TCCAAAATACAATAGTGGG |
| 2991 | CCACTATTGTATTTTGGAA | 2992 | TTCCAAAATACAATAGTGG |
| 2993 | CACTATTGTATTTTGGAAG | 2994 | CTTCCAAAATACAATAGTG |
| 2995 | ACTATTGTATTTTGGAAGC | 2996 | GCTTCCAAAATACAATAGT |
| 2997 | CTATTGTATTTTGGAAGCA | 2998 | TGCTTCCAAAATACAATAG |
| 2999 | TATTGTATTTTGGAAGCAC | 3000 | GTGCTTCCAAAATACAATA |
| 3001 | ATTGTATTTTGGAAGCACA | 3002 | TGTGCTTCCAAAATACAAT |
| 3003 | TTGTATTTTGGAAGCACAT | 3004 | ATGTGCTTCCAAAATACAA |
| 3005 | TGTATTTTGGAAGCACATA | 3006 | TATGTGCTTCCAAAATACA |
| 3007 | GTATTTTGGAAGCACATAA | 3008 | TTATGTGCTTCCAAAATAC |
| 3009 | TATTTTGGAAGCACATAAC | 3010 | GTTATGTGCTTCCAAAATA |
| 3011 | ATTTTGGAAGCACATAACT | 3012 | AGTTATGTGCTTCCAAAAT |
| 3013 | TTTTGGAAGCACATAACTT | 3014 | AAGTTATGTGCTTCCAAAA |
| 3015 | TTTGGAAGCACATAACTTG | 3016 | CAAGTTATGTGCTTCCAAA |
| 3017 | TTGGAAGCACATAACTTGT | 3018 | ACAAGTTATGTGCTTCCAA |
| 3019 | TGGAAGCACATAACTTGTT | 3020 | AACAAGTTATGTGCTTCCA |
| 3021 | GGAAGCACATAACTTGTTT | 3022 | AAACAAGTTATGTGCTTCC |
| 3023 | GAAGCACATAACTTGTTTG | 3024 | CAAACAAGTTATGTGCTTC |
| 3025 | AAGCACATAACTTGTTTGG | 3026 | CCAAACAAGTTATGTGCTT |
| 3027 | AGCACATAACTTGTTTGGT | 3028 | ACCAAACAAGTTATGTGCT |
| 3029 | GCACATAACTTGTTTGGTT | 3030 | AACCAAACAAGTTATGTGC |
| 3031 | CACATAACTTGTTTGGTTT | 3032 | AAACCAAACAAGTTATGTG |
| 3033 | ACATAACTTGTTTGGTTTC | 3034 | GAAACCAAACAAGTTATGT |
| 3035 | CATAACTTGTTTGGTTTCA | 3036 | TGAAACCAAACAAGTTATG |
| 3037 | ATAACTTGTTTGGTTTCAC | 3038 | GTGAAACCAAACAAGTTAT |
| 3039 | TAACTTGTTTGGTTTCACA | 3040 | TGTGAAACCAAACAAGTTA |
| 3041 | AACTTGTTTGGTTTCACAG | 3042 | CTGTGAAACCAAACAAGTT |
| 3043 | ACTTGTTTGGTTTCACAGG | 3044 | CCTGTGAAACCAAACAAGT |
| 3045 | CTTGTTTGGTTTCACAGGT | 3046 | ACCTGTGAAACCAAACAAG |
| 3047 | TTGTTTGGTTTCACAGGTT | 3048 | AACCTGTGAAACCAAACAA |
| 3049 | TGTTTGGTTTCACAGGTTC | 3050 | GAACCTGTGAAACCAAACA |
| 3051 | GTTTGGTTTCACAGGTTCA | 3052 | TGAACCTGTGAAACCAAAC |
| 3053 | TTTGGTTTCACAGGTTCAC | 3054 | GTGAACCTGTGAAACCAAA |
| 3055 | TTGGTTTCACAGGTTCACA | 3056 | TGTGAACCTGTGAAACCAA |
| 3057 | TGGTTTCACAGGTTCACAG | 3058 | CTGTGAACCTGTGAAACCA |
| 3059 | GGTTTCACAGGTTCACAGT | 3060 | ACTGTGAACCTGTGAAACC |
| 3061 | GTTTCACAGGTTCACAGTT | 3062 | AACTGTGAACCTGTGAAAC |
| 3063 | TTTCACAGGTTCACAGTTA | 3064 | TAACTGTGAACCTGTGAAA |
| 3065 | TTCACAGGTTCACAGTTAA | 3066 | TTAACTGTGAACCTGTGAA |
| 3067 | TCACAGGTTCACAGTTAAG | 3068 | CTTAACTGTGAACCTGTGA |
| 3069 | CACAGGTTCACAGTTAAGA | 3070 | TCTTAACTGTGAACCTGTG |
| 3071 | ACAGGTTCACAGTTAAGAA | 3072 | TTCTTAACTGTGAACCTGT |
| 3073 | CAGGTTCACAGTTAAGAAG | 3074 | CTTCTTAACTGTGAACCTG |
| 3075 | AGGTTCACAGTTAAGAAGG | 3076 | CCTTCTTAACTGTGAACCT |
| 3077 | GGTTCACAGTTAAGAAGGA | 3078 | TCCTTCTTAACTGTGAACC |
| 3079 | GTTCACAGTTAAGAAGGAA | 3080 | TTCCTTCTTAACTGTGAAC |
| 3081 | TTCACAGTTAAGAAGGAAT | 3082 | ATTCCTTCTTAACTGTGAA |
| 3083 | TCACAGTTAAGAAGGAATT | 3084 | AATTCCTTCTTAACTGTGA |
| 3085 | CACAGTTAAGAAGGAATTT | 3086 | AAATTCCTTCTTAACTGTG |
| 3087 | ACAGTTAAGAAGGAATTTT | 3088 | AAAATTCCTTCTTAACTGT |
| 3089 | CAGTTAAGAAGGAATTTTG | 3090 | CAAAATTCCTTCTTAACTG |
| 3091 | AGTTAAGAAGGAATTTTGC | 3092 | GCAAAATTCCTTCTTAACT |
| 3093 | GTTAAGAAGGAATTTTGCC | 3094 | GGCAAAATTCCTTCTTAAC |
| 3095 | TTAAGAAGGAATTTTGCCT | 3096 | AGGCAAAATTCCTTCTTAA |
| 3097 | TAAGAAGGAATTTTGCCTC | 3098 | GAGGCAAAATTCCTTCTTA |
| 3099 | AAGAAGGAATTTTGCCTCT | 3100 | AGAGGCAAAATTCCTTCTT |
| 3101 | AGAAGGAATTTTGCCTCTG | 3102 | CAGAGGCAAAATTCCTTCT |
| 3103 | GAAGGAATTTTGCCTCTGA | 3104 | TCAGAGGCAAAATTCCTTC |
| 3105 | AAGGAATTTTGCCTCTGAA | 3106 | TTCAGAGGCAAAATTCCTT |
| 3107 | AGGAATTTTGCCTCTGAAT | 3108 | ATTCAGAGGCAAAATTCCT |
| 3109 | GGAATTTTGCCTCTGAATA | 3110 | TATTCAGAGGCAAAATTCC |
| 3111 | GAATTTTGCCTCTGAATAA | 3112 | TTATTCAGAGGCAAAATTC |
| 3113 | AATTTTGCCTCTGAATAAA | 3114 | TTTATTCAGAGGCAAAATT |
| 3115 | ATTTTGCCTCTGAATAAAT | 3116 | ATTTATTCAGAGGCAAAAT |
| 3117 | TTTTGCCTCTGAATAAATA | 3118 | TATTTATTCAGAGGCAAAA |
| 3119 | TTTGCCTCTGAATAAATAG | 3120 | CTATTTATTCAGAGGCAAA |
| 3121 | TTGCCTCTGAATAAATAGA | 3122 | TCTATTTATTCAGAGGCAA |
| 3123 | TGCCTCTGAATAAATAGAA | 3124 | TTCTATTTATTCAGAGGCA |
| 3125 | GCCTCTGAATAAATAGAAT | 3126 | ATTCTATTTATTCAGAGGC |
| 3127 | CCTCTGAATAAATAGAATC | 3128 | GATTCTATTTATTCAGAGG |
| 3129 | CTCTGAATAAATAGAATCT | 3130 | AGATTCTATTTATTCAGAG |
| 3131 | TCTGAATAAATAGAATCTT | 3132 | AAGATTCTATTTATTCAGA |
| 3133 | CTGAATAAATAGAATCTTG | 3134 | CAAGATTCTATTTATTCAG |
| 3135 | TGAATAAATAGAATCTTGA | 3136 | TCAAGATTCTATTTATTCA |
| 3137 | GAATAAATAGAATCTTGAG | 3138 | CTCAAGATTCTATTTATTC |
| 3139 | AATAAATAGAATCTTGAGT | 3140 | ACTCAAGATTCTATTTATT |
| 3141 | ATAAATAGAATCTTGAGTC | 3142 | GACTCAAGATTCTATTTAT |
| 3143 | TAAATAGAATCTTGAGTCT | 3144 | AGACTCAAGATTCTATTTA |
| 3145 | AAATAGAATCTTGAGTCTC | 3146 | GAGACTCAAGATTCTATTT |
| 3147 | AATAGAATCTTGAGTCTCA | 3148 | TGAGACTCAAGATTCTATT |
| 3149 | ATAGAATCTTGAGTCTCAT | 3150 | ATGAGACTCAAGATTCTAT |
| 3151 | TAGAATCTTGAGTCTCATG | 3152 | CATGAGACTCAAGATTCTA |
| 3153 | AGAATCTTGAGTCTCATGC | 3154 | GCATGAGACTCAAGATTCT |

TABLE 11
Human RAET1L NM_130900

| SEQID NO. | siRNA (19bp) | SEQID NO. | Reverse complement |
|---|---|---|---|
| 3155 | GATTTCATCTTCCAGGATC | 3156 | GATCCTGGAAGATGAAATC |
| 3157 | ATTTCATCTTCCAGGATCC | 3158 | GGATCCTGGAAGATGAAAT |
| 3159 | TTTCATCTTCCAGGATCCA | 3160 | TGGATCCTGGAAGATGAAA |
| 3161 | TTCATCTTCCAGGATCCAC | 3162 | GTGGATCCTGGAAGATGAA |
| 3163 | TCATCTTCCAGGATCCACC | 3164 | GGTGGATCCTGGAAGATGA |
| 3165 | CATCTTCCAGGATCCACCT | 3166 | AGGTGGATCCTGGAAGATG |
| 3167 | ATCTTCCAGGATCCACCTT | 3168 | AAGGTGGATCCTGGAAGAT |
| 3169 | TCTTCCAGGATCCACCTTG | 3170 | CAAGGTGGATCCTGGAAGA |
| 3171 | CTTCCAGGATCCACCTTGA | 3172 | TCAAGGTGGATCCTGGAAG |
| 3173 | TTCCAGGATCCACCTTGAT | 3174 | ATCAAGGTGGATCCTGGAA |
| 3175 | TCCAGGATCCACCTTGATT | 3176 | AATCAAGGTGGATCCTGGA |
| 3177 | CCAGGATCCACCTTGATTA | 3178 | TAATCAAGGTGGATCCTGG |
| 3179 | CAGGATCCACCTTGATTAA | 3180 | TTAATCAAGGTGGATCCTG |
| 3181 | AGGATCCACCTTGATTAAA | 3182 | TTTAATCAAGGTGGATCCT |
| 3183 | GGATCCACCTTGATTAAAT | 3184 | ATTTAATCAAGGTGGATCC |
| 3185 | GATCCACCTTGATTAAATC | 3186 | GATTTAATCAAGGTGGATC |
| 3187 | ATCCACCTTGATTAAATCT | 3188 | AGATTTAATCAAGGTGGAT |
| 3189 | TCCACCTTGATTAAATCTC | 3190 | GAGATTTAATCAAGGTGGA |
| 3191 | CCACCTTGATTAAATCTCT | 3192 | AGAGATTTAATCAAGGTGG |
| 3193 | CACCTTGATTAAATCTCTT | 3194 | AAGAGATTTAATCAAGGTG |
| 3195 | ACCTTGATTAAATCTCTTG | 3196 | CAAGAGATTTAATCAAGGT |
| 3197 | CCTTGATTAAATCTCTTGT | 3198 | ACAAGAGATTTAATCAAGG |
| 3199 | CTTGATTAAATCTCTTGTC | 3200 | GACAAGAGATTTAATCAAG |
| 3201 | TTGATTAAATCTCTTGTCC | 3202 | GGACAAGAGATTTAATCAA |
| 3203 | TGATTAAATCTCTTGTCCC | 3204 | GGGACAAGAGATTTAATCA |
| 3205 | GATTAAATCTCTTGTCCCC | 3206 | GGGGACAAGAGATTTAATC |
| 3207 | ATTAAATCTCTTGTCCCCA | 3208 | TGGGGACAAGAGATTTAAT |
| 3209 | TTAAATCTCTTGTCCCCAG | 3210 | CTGGGGACAAGAGATTTAA |
| 3211 | TAAATCTCTTGTCCCCAGC | 3212 | GCTGGGGACAAGAGATTTA |
| 3213 | AAATCTCTTGTCCCCAGCC | 3214 | GGCTGGGGACAAGAGATTT |
| 3215 | AATCTCTTGTCCCCAGCCC | 3216 | GGGCTGGGGACAAGAGATT |
| 3217 | ATCTCTTGTCCCCAGCCCT | 3218 | AGGGCTGGGGACAAGAGAT |
| 3219 | TCTCTTGTCCCCAGCCCTC | 3220 | GAGGGCTGGGGACAAGAGA |
| 3221 | CTCTTGTCCCCAGCCCTCC | 3222 | GGAGGGCTGGGGACAAGAG |
| 3223 | TCTTGTCCCCAGCCCTCCT | 3224 | AGGAGGGCTGGGGACAAGA |
| 3225 | CTTGTCCCCAGCCCTCCTG | 3226 | CAGGAGGGCTGGGGACAAG |
| 3227 | TTGTCCCCAGCCCTCCTGG | 3228 | CCAGGAGGGCTGGGGACAA |
| 3229 | TGTCCCCAGCCCTCCTGGT | 3230 | ACCAGGAGGGCTGGGGACA |
| 3231 | GTCCCCAGCCCTCCTGGTC | 3232 | GACCAGGAGGGCTGGGGAC |
| 3233 | TCCCCAGCCCTCCTGGTCC | 3234 | GGACCAGGAGGGCTGGGGA |
| 3235 | CCCCAGCCCTCCTGGTCCC | 3236 | GGGACCAGGAGGGCTGGGG |
| 3237 | CCCAGCCCTCCTGGTCCCC | 3238 | GGGGACCAGGAGGGCTGGG |
| 3239 | CCAGCCCTCCTGGTCCCCA | 3240 | TGGGGACCAGGAGGGCTGG |
| 3241 | CAGCCCTCCTGGTCCCCAA | 3242 | TTGGGGACCAGGAGGGCTG |
| 3243 | AGCCCTCCTGGTCCCCAAT | 3244 | ATTGGGGACCAGGAGGGCT |
| 3245 | GCCCTCCTGGTCCCCAATG | 3246 | CATTGGGGACCAGGAGGGC |
| 3247 | CCCTCCTGGTCCCCAATGG | 3248 | CCATTGGGGACCAGGAGGG |
| 3249 | CCTCCTGGTCCCCAATGGC | 3250 | GCCATTGGGGACCAGGAGG |
| 3251 | CTCCTGGTCCCCAATGGCA | 3252 | TGCCATTGGGGACCAGGAG |
| 3253 | TCCTGGTCCCCAATGGCAG | 3254 | CTGCCATTGGGGACCAGGA |
| 3255 | CCTGGTCCCCAATGGCAGC | 3256 | GCTGCCATTGGGGACCAGG |
| 3257 | CTGGTCCCCAATGGCAGCA | 3258 | TGCTGCCATTGGGGACCAG |
| 3259 | TGGTCCCCAATGGCAGCAG | 3260 | CTGCTGCCATTGGGGACCA |
| 3261 | GGTCCCCAATGGCAGCAGC | 3262 | GCTGCTGCCATTGGGGACC |
| 3263 | GTCCCCAATGGCAGCAGCC | 3264 | GGCTGCTGCCATTGGGGAC |
| 3265 | TCCCCAATGGCAGCAGCCG | 3266 | CGGCTGCTGCCATTGGGGA |
| 3267 | CCCCAATGGCAGCAGCCGC | 3268 | GCGGCTGCTGCCATTGGGG |
| 3269 | CCCAATGGCAGCAGCCGCC | 3270 | GGCGGCTGCTGCCATTGGG |
| 3271 | CCAATGGCAGCAGCCGCCA | 3272 | TGGCGGCTGCTGCCATTGG |
| 3273 | CAATGGCAGCAGCCGCCAT | 3274 | ATGGCGGCTGCTGCCATTG |
| 3275 | AATGGCAGCAGCCGCCATC | 3276 | GATGGCGGCTGCTGCCATT |
| 3277 | ATGGCAGCAGCCGCCATCC | 3278 | GGATGGCGGCTGCTGCCAT |
| 3279 | TGGCAGCAGCCGCCATCCC | 3280 | GGGATGGCGGCTGCTGCCA |
| 3281 | GGCAGCAGCCGCCATCCCA | 3282 | TGGGATGGCGGCTGCTGCC |
| 3283 | GCAGCAGCCGCCATCCCAG | 3284 | CTGGGATGGCGGCTGCTGC |
| 3285 | CAGCAGCCGCCATCCCAGC | 3286 | GCTGGGATGGCGGCTGCTG |
| 3287 | AGCAGCCGCCATCCCAGCT | 3288 | AGCTGGGATGGCGGCTGCT |
| 3289 | GCAGCCGCCATCCCAGCTT | 3290 | AAGCTGGGATGGCGGCTGC |
| 3291 | CAGCCGCCATCCCAGCTTT | 3292 | AAAGCTGGGATGGCGGCTG |
| 3293 | AGCCGCCATCCCAGCTTTG | 3294 | CAAAGCTGGGATGGCGGCT |
| 3295 | GCCGCCATCCCAGCTTTGC | 3296 | GCAAAGCTGGGATGGCGGC |
| 3297 | CCGCCATCCCAGCTTTGCT | 3298 | AGCAAAGCTGGGATGGCGG |
| 3299 | CGCCATCCCAGCTTTGCTT | 3300 | AAGCAAAGCTGGGATGGCG |
| 3301 | GCCATCCCAGCTTTGCTTC | 3302 | GAAGCAAAGCTGGGATGGC |
| 3303 | CCATCCCAGCTTTGCTTCT | 3304 | AGAAGCAAAGCTGGGATGG |
| 3305 | CATCCCAGCTTTGCTTCTG | 3306 | CAGAAGCAAAGCTGGGATG |
| 3307 | ATCCCAGCTTTGCTTCTGT | 3308 | ACAGAAGCAAAGCTGGGAT |
| 3309 | TCCCAGCTTTGCTTCTGTG | 3310 | CACAGAAGCAAAGCTGGGA |
| 3311 | CCCAGCTTTGCTTCTGTGC | 3312 | GCACAGAAGCAAAGCTGGG |
| 3313 | CCAGCTTTGCTTCTGTGCC | 3314 | GGCACAGAAGCAAAGCTGG |
| 3315 | CAGCTTTGCTTCTGTGCCT | 3316 | AGGCACAGAAGCAAAGCTG |
| 3317 | AGCTTTGCTTCTGTGCCTC | 3318 | GAGGCACAGAAGCAAAGCT |
| 3319 | GCTTTGCTTCTGTGCCTCC | 3320 | GGAGGCACAGAAGCAAAGC |
| 3321 | CTTTGCTTCTGTGCCTCCC | 3322 | GGGAGGCACAGAAGCAAAG |
| 3323 | TTTGCTTCTGTGCCTCCCG | 3324 | CGGGAGGCACAGAAGCAAA |
| 3325 | TTGCTTCTGTGCCTCCCGC | 3326 | GCGGGAGGCACAGAAGCAA |
| 3327 | TGCTTCTGTGCCTCCCGCT | 3328 | AGCGGGAGGCACAGAAGCA |
| 3329 | GCTTCTGTGCCTCCCGCTT | 3330 | AAGCGGGAGGCACAGAAGC |
| 3331 | CTTCTGTGCCTCCCGCTTC | 3332 | GAAGCGGGAGGCACAGAAG |
| 3333 | TTCTGTGCCTCCCGCTTCT | 3334 | AGAAGCGGGAGGCACAGAA |
| 3335 | TCTGTGCCTCCCGCTTCTG | 3336 | CAGAAGCGGGAGGCACAGA |
| 3337 | CTGTGCCTCCCGCTTCTGT | 3338 | ACAGAAGCGGGAGGCACAG |
| 3339 | TGTGCCTCCCGCTTCTGTT | 3340 | AACAGAAGCGGGAGGCACA |
| 3341 | GTGCCTCCCGCTTCTGTTC | 3342 | GAACAGAAGCGGGAGGCAC |
| 3343 | TGCCTCCCGCTTCTGTTCC | 3344 | GGAACAGAAGCGGGAGGCA |
| 3345 | GCCTCCCGCTTCTGTTCCT | 3346 | AGGAACAGAAGCGGGAGGC |
| 3347 | CCTCCCGCTTCTGTTCCTG | 3348 | CAGGAACAGAAGCGGGAGG |
| 3349 | CTCCCGCTTCTGTTCCTGC | 3350 | GCAGGAACAGAAGCGGGAG |
| 3351 | TCCCGCTTCTGTTCCTGCT | 3352 | AGCAGGAACAGAAGCGGGA |
| 3353 | CCCGCTTCTGTTCCTGCTG | 3354 | CAGCAGGAACAGAAGCGGG |
| 3355 | CCGCTTCTGTTCCTGCTGT | 3356 | ACAGCAGGAACAGAAGCGG |
| 3357 | CGCTTCTGTTCCTGCTGTT | 3358 | AACAGCAGGAACAGAAGCG |
| 3359 | GCTTCTGTTCCTGCTGTTC | 3360 | GAACAGCAGGAACAGAAGC |
| 3361 | CTTCTGTTCCTGCTGTTCG | 3362 | CGAACAGCAGGAACAGAAG |
| 3363 | TTCTGTTCCTGCTGTTCGG | 3364 | CCGAACAGCAGGAACAGAA |
| 3365 | TCTGTTCCTGCTGTTCGGC | 3366 | GCCGAACAGCAGGAACAGA |
| 3367 | CTGTTCCTGCTGTTCGGCT | 3368 | AGCCGAACAGCAGGAACAG |
| 3369 | TGTTCCTGCTGTTCGGCTG | 3370 | CAGCCGAACAGCAGGAACA |
| 3371 | GTTCCTGCTGTTCGGCTGG | 3372 | CCAGCCGAACAGCAGGAAC |
| 3373 | TTCCTGCTGTTCGGCTGGT | 3374 | ACCAGCCGAACAGCAGGAA |
| 3375 | TCCTGCTGTTCGGCTGGTC | 3376 | GACCAGCCGAACAGCAGGA |
| 3377 | CCTGCTGTTCGGCTGGTCC | 3378 | GGACCAGCCGAACAGCAGG |
| 3379 | CTGCTGTTCGGCTGGTCCC | 3380 | GGGACCAGCCGAACAGCAG |
| 3381 | TGCTGTTCGGCTGGTCCCG | 3382 | CGGGACCAGCCGAACAGCA |
| 3383 | GCTGTTCGGCTGGTCCCGG | 3384 | CCGGGACCAGCCGAACAGC |
| 3385 | CTGTTCGGCTGGTCCCGGG | 3386 | CCCGGGACCAGCCGAACAG |
| 3387 | TGTTCGGCTGGTCCCGGGC | 3388 | GCCCGGGACCAGCCGAACA |
| 3389 | GTTCGGCTGGTCCCGGGCT | 3390 | AGCCCGGGACCAGCCGAAC |
| 3391 | TTCGGCTGGTCCCGGGCTA | 3392 | TAGCCCGGGACCAGCCGAA |
| 3393 | TCGGCTGGTCCCGGGCTAG | 3394 | CTAGCCCGGGACCAGCCGA |
| 3395 | CGGCTGGTCCCGGGCTAGG | 3396 | CCTAGCCCGGGACCAGCCG |
| 3397 | GGCTGGTCCCGGGCTAGGC | 3398 | GCCTAGCCCGGGACCAGCC |
| 3399 | GCTGGTCCCGGGCTAGGCG | 3400 | CGCCTAGCCCGGGACCAGC |
| 3401 | CTGGTCCCGGGCTAGGCGA | 3402 | TCGCCTAGCCCGGGACCAG |
| 3403 | TGGTCCCGGGCTAGGCGAG | 3404 | CTCGCCTAGCCCGGGACCA |
| 3405 | GGTCCCGGGCTAGGCGAGA | 3406 | TCTCGCCTAGCCCGGGACC |
| 3407 | GTCCCGGGCTAGGCGAGAC | 3408 | GTCTCGCCTAGCCCGGGAC |
| 3409 | TCCCGGGCTAGGCGAGACG | 3410 | CGTCTCGCCTAGCCCGGGA |
| 3411 | CCCGGGCTAGGCGAGACGA | 3412 | TCGTCTCGCCTAGCCCGGG |
| 3413 | CCGGGCTAGGCGAGACGAC | 3414 | GTCGTCTCGCCTAGCCCGG |
| 3415 | CGGGCTAGGCGAGACGACC | 3416 | GGTCGTCTCGCCTAGCCCG |
| 3417 | GGGCTAGGCGAGACGACCC | 3418 | GGGTCGTCTCGCCTAGCCC |
| 3419 | GGCTAGGCGAGACGACCCT | 3420 | AGGGTCGTCTCGCCTAGCC |
| 3421 | GCTAGGCGAGACGACCCTC | 3422 | GAGGGTCGTCTCGCCTAGC |
| 3423 | CTAGGCGAGACGACCCTCA | 3424 | TGAGGGTCGTCTCGCCTAG |
| 3425 | TAGGCGAGACGACCCTCAC | 3426 | GTGAGGGTCGTCTCGCCTA |
| 3427 | AGGCGAGACGACCCTCACT | 3428 | AGTGAGGGTCGTCTCGCCT |
| 3429 | GGCGAGACGACCCTCACTC | 3430 | GAGTGAGGGTCGTCTCGCC |
| 3431 | GCGAGACGACCCTCACTCT | 3432 | AGAGTGAGGGTCGTCTCGC |
| 3433 | CGAGACGACCCTCACTCTC | 3434 | GAGAGTGAGGGTCGTCTCG |
| 3435 | GAGACGACCCTCACTCTCT | 3436 | AGAGAGTGAGGGTCGTCTC |
| 3437 | AGACGACCCTCACTCTCTT | 3438 | AAGAGAGTGAGGGTCGTCT |
| 3439 | GACGACCCTCACTCTCTTT | 3440 | AAAGAGAGTGAGGGTCGTC |
| 3441 | ACGACCCTCACTCTCTTTG | 3442 | CAAAGAGAGTGAGGGTCGT |
| 3443 | CGACCCTCACTCTCTTTGC | 3444 | GCAAAGAGAGTGAGGGTCG |
| 3445 | GACCCTCACTCTCTTTGCT | 3446 | AGCAAAGAGAGTGAGGGTC |
| 3447 | ACCCTCACTCTCTTTGCTA | 3448 | TAGCAAAGAGAGTGAGGGT |
| 3449 | CCCTCACTCTCTTTGCTAT | 3450 | ATAGCAAAGAGAGTGAGGG |
| 3451 | CCTCACTCTCTTTGCTATG | 3452 | CATAGCAAAGAGAGTGAGG |
| 3453 | CTCACTCTCTTTGCTATGA | 3454 | TCATAGCAAAGAGAGTGAG |
| 3455 | TCACTCTCTTTGCTATGAC | 3456 | GTCATAGCAAAGAGAGTGA |
| 3457 | CACTCTCTTTGCTATGACA | 3458 | TGTCATAGCAAAGAGAGTG |
| 3459 | ACTCTCTTTGCTATGACAT | 3460 | ATGTCATAGCAAAGAGAGT |
| 3461 | CTCTCTTTGCTATGACATC | 3462 | GATGTCATAGCAAAGAGAG |
| 3463 | TCTCTTTGCTATGACATCA | 3464 | TGATGTCATAGCAAAGAGA |
| 3465 | CTCTTTGCTATGACATCAC | 3466 | GTGATGTCATAGCAAAGAG |
| 3467 | TCTTTGCTATGACATCACC | 3468 | GGTGATGTCATAGCAAAGA |
| 3469 | CTTTGCTATGACATCACCG | 3470 | CGGTGATGTCATAGCAAAG |
| 3471 | TTTGCTATGACATCACCGT | 3472 | ACGGTGATGTCATAGCAAA |
| 3473 | TTGCTATGACATCACCGTC | 3474 | GACGGTGATGTCATAGCAA |
| 3475 | TGCTATGACATCACCGTCA | 3476 | TGACGGTGATGTCATAGCA |
| 3477 | GCTATGACATCACCGTCAT | 3478 | ATGACGGTGATGTCATAGC |
| 3479 | CTATGACATCACCGTCATC | 3480 | GATGACGGTGATGTCATAG |
| 3481 | TATGACATCACCGTCATCC | 3482 | GGATGACGGTGATGTCATA |
| 3483 | ATGACATCACCGTCATCCC | 3484 | GGGATGACGGTGATGTCAT |
| 3485 | TGACATCACCGTCATCCCT | 3486 | AGGGATGACGGTGATGTCA |
| 3487 | GACATCACCGTCATCCCTA | 3488 | TAGGGATGACGGTGATGTC |
| 3489 | ACATCACCGTCATCCCTAA | 3490 | TTAGGGATGACGGTGATGT |
| 3491 | CATCACCGTCATCCCTAAG | 3492 | CTTAGGGATGACGGTGATG |
| 3493 | ATCACCGTCATCCCTAAGT | 3494 | ACTTAGGGATGACGGTGAT |
| 3495 | TCACCGTCATCCCTAAGTT | 3496 | AACTTAGGGATGACGGTGA |
| 3497 | CACCGTCATCCCTAAGTTC | 3498 | GAACTTAGGGATGACGGTG |
| 3499 | ACCGTCATCCCTAAGTTCA | 3500 | TGAACTTAGGGATGACGGT |
| 3501 | CCGTCATCCCTAAGTTCAG | 3502 | CTGAACTTAGGGATGACGG |
| 3503 | CGTCATCCCTAAGTTCAGA | 3504 | TCTGAACTTAGGGATGACG |
| 3505 | GTCATCCCTAAGTTCAGAC | 3506 | GTCTGAACTTAGGGATGAC |
| 3507 | TCATCCCTAAGTTCAGACC | 3508 | GGTCTGAACTTAGGGATGA |
| 3509 | CATCCCTAAGTTCAGACCT | 3510 | AGGTCTGAACTTAGGGATG |
| 3511 | ATCCCTAAGTTCAGACCTG | 3512 | CAGGTCTGAACTTAGGGAT |
| 3513 | TCCCTAAGTTCAGACCTGG | 3514 | CCAGGTCTGAACTTAGGGA |
| 3515 | CCCTAAGTTCAGACCTGGA | 3516 | TCCAGGTCTGAACTTAGGG |
| 3517 | CCTAAGTTCAGACCTGGAC | 3518 | GTCCAGGTCTGAACTTAGG |
| 3519 | CTAAGTTCAGACCTGGACC | 3520 | GGTCCAGGTCTGAACTTAG |
| 3521 | TAAGTTCAGACCTGGACCA | 3522 | TGGTCCAGGTCTGAACTTA |
| 3523 | AAGTTCAGACCTGGACCAC | 3524 | GTGGTCCAGGTCTGAACTT |
| 3525 | AGTTCAGACCTGGACCACG | 3526 | CGTGGTCCAGGTCTGAACT |
| 3527 | GTTCAGACCTGGACCACGG | 3528 | CCGTGGTCCAGGTCTGAAC |
| 3529 | TTCAGACCTGGACCACGGT | 3530 | ACCGTGGTCCAGGTCTGAA |
| 3531 | TCAGACCTGGACCACGGTG | 3532 | CACCGTGGTCCAGGTCTGA |
| 3533 | CAGACCTGGACCACGGTGG | 3534 | CCACCGTGGTCCAGGTCTG |
| 3535 | AGACCTGGACCACGGTGGT | 3536 | ACCACCGTGGTCCAGGTCT |
| 3537 | GACCTGGACCACGGTGGTG | 3538 | CACCACCGTGGTCCAGGTC |
| 3539 | ACCTGGACCACGGTGGTGT | 3540 | ACACCACCGTGGTCCAGGT |
| 3541 | CCTGGACCACGGTGGTGTG | 3542 | CACACCACCGTGGTCCAGG |
| 3543 | CTGGACCACGGTGGTGTGC | 3544 | GCACACCACCGTGGTCCAG |
| 3545 | TGGACCACGGTGGTGTGCG | 3546 | CGCACACCACCGTGGTCCA |
| 3547 | GGACCACGGTGGTGTGCGG | 3548 | CCGCACACCACCGTGGTCC |
| 3549 | GACCACGGTGGTGTGCGGT | 3550 | ACCGCACACCACCGTGGTC |
| 3551 | ACCACGGTGGTGTGCGGTT | 3552 | AACCGCACACCACCGTGGT |
| 3553 | CCACGGTGGTGTGCGGTTC | 3554 | GAACCGCACACCACCGTGG |
| 3555 | CACGGTGGTGTGCGGTTCA | 3556 | TGAACCGCACACCACCGTG |
| 3557 | ACGGTGGTGTGCGGTTCAA | 3558 | TTGAACCGCACACCACCGT |
| 3559 | CGGTGGTGTGCGGTTCAAG | 3560 | CTTGAACCGCACACCACCG |
| 3561 | GGTGGTGTGCGGTTCAAGG | 3562 | CCTTGAACCGCACACCACC |
| 3563 | GTGGTGTGCGGTTCAAGGC | 3564 | GCCTTGAACCGCACACCAC |
| 3565 | TGGTGTGCGGTTCAAGGCC | 3566 | GGCCTTGAACCGCACACCA |
| 3567 | GGTGTGCGGTTCAAGGCCA | 3568 | TGGCCTTGAACCGCACACC |
| 3569 | GTGTGCGGTTCAAGGCCAG | 3570 | CTGGCCTTGAACCGCACAC |
| 3571 | TGTGCGGTTCAAGGCCAGG | 3572 | CCTGGCCTTGAACCGCACA |
| 3573 | GTGCGGTTCAAGGCCAGGT | 3574 | ACCTGGCCTTGAACCGCAC |
| 3575 | TGCGGTTCAAGGCCAGGTG | 3576 | CACCTGGCCTTGAACCGCA |
| 3577 | GCGGTTCAAGGCCAGGTGG | 3578 | CCACCTGGCCTTGAACCGC |
| 3579 | CGGTTCAAGGCCAGGTGGA | 3580 | TCCACCTGGCCTTGAACCG |
| 3581 | GGTTCAAGGCCAGGTGGAT | 3582 | ATCCACCTGGCCTTGAACC |
| 3583 | GTTCAAGGCCAGGTGGATG | 3584 | CATCCACCTGGCCTTGAAC |
| 3585 | TTCAAGGCCAGGTGGATGA | 3586 | TCATCCACCTGGCCTTGAA |
| 3587 | TCAAGGCCAGGTGGATGAA | 3588 | TTCATCCACCTGGCCTTGA |
| 3589 | CAAGGCCAGGTGGATGAAA | 3590 | TTTCATCCACCTGGCCTTG |
| 3591 | AAGGCCAGGTGGATGAAAA | 3592 | TTTTCATCCACCTGGCCTT |
| 3593 | AGGCCAGGTGGATGAAAAG | 3594 | CTTTTCATCCACCTGGCCT |
| 3595 | GGCCAGGTGGATGAAAAGA | 3596 | TCTTTTCATCCACCTGGCC |
| 3597 | GCCAGGTGGATGAAAAGAC | 3598 | GTCTTTTCATCCACCTGGC |
| 3599 | CCAGGTGGATGAAAAGACT | 3600 | AGTCTTTTCATCCACCTGG |
| 3601 | CAGGTGGATGAAAAGACTT | 3602 | AAGTCTTTTCATCCACCTG |
| 3603 | AGGTGGATGAAAAGACTTT | 3604 | AAAGTCTTTTCATCCACCT |
| 3605 | GGTGGATGAAAAGACTTTT | 3606 | AAAAGTCTTTTCATCCACC |
| 3607 | GTGGATGAAAAGACTTTTC | 3608 | GAAAAGTCTTTTCATCCAC |
| 3609 | TGGATGAAAAGACTTTTCT | 3610 | AGAAAAGTCTTTTCATCCA |
| 3611 | GGATGAAAAGACTTTTCTT | 3612 | AAGAAAAGTCTTTTCATCC |
| 3613 | GATGAAAAGACTTTTCTTC | 3614 | GAAGAAAAGTCTTTTCATC |
| 3615 | ATGAAAAGACTTTTCTTCA | 3616 | TGAAGAAAAGTCTTTTCAT |
| 3617 | TGAAAAGACTTTTCTTCAC | 3618 | GTGAAGAAAAGTCTTTTCA |
| 3619 | GAAAAGACTTTTCTTCACT | 3620 | AGTGAAGAAAAGTCTTTTC |
| 3621 | AAAAGACTTTTCTTCACTA | 3622 | TAGTGAAGAAAAGTCTTTT |
| 3623 | AAAGACTTTTCTTCACTAT | 3624 | ATAGTGAAGAAAAGTCTTT |
| 3625 | AAGACTTTTCTTCACTATG | 3626 | CATAGTGAAGAAAAGTCTT |
| 3627 | AGACTTTTCTTCACTATGA | 3628 | TCATAGTGAAGAAAAGTCT |
| 3629 | GACTTTTCTTCACTATGAC | 3630 | GTCATAGTGAAGAAAAGTC |
| 3631 | ACTTTTCTTCACTATGACT | 3632 | AGTCATAGTGAAGAAAAGT |
| 3633 | CTTTTCTTCACTATGACTG | 3634 | CAGTCATAGTGAAGAAAAG |
| 3635 | TTTTCTTCACTATGACTGT | 3636 | ACAGTCATAGTGAAGAAAA |
| 3637 | TTTCTTCACTATGACTGTG | 3638 | CACAGTCATAGTGAAGAAA |
| 3639 | TTCTTCACTATGACTGTGG | 3640 | CCACAGTCATAGTGAAGAA |
| 3641 | TCTTCACTATGACTGTGGC | 3642 | GCCACAGTCATAGTGAAGA |
| 3643 | CTTCACTATGACTGTGGCA | 3644 | TGCCACAGTCATAGTGAAG |
| 3645 | TTCACTATGACTGTGGCAA | 3646 | TTGCCACAGTCATAGTGAA |
| 3647 | TCACTATGACTGTGGCAAC | 3648 | GTTGCCACAGTCATAGTGA |
| 3649 | CACTATGACTGTGGCAACA | 3650 | TGTTGCCACAGTCATAGTG |
| 3651 | ACTATGACTGTGGCAACAA | 3652 | TTGTTGCCACAGTCATAGT |
| 3653 | CTATGACTGTGGCAACAAG | 3654 | CTTGTTGCCACAGTCATAG |
| 3655 | TATGACTGTGGCAACAAGA | 3656 | TCTTGTTGCCACAGTCATA |
| 3657 | ATGACTGTGGCAACAAGAC | 3658 | GTCTTGTTGCCACAGTCAT |
| 3659 | TGACTGTGGCAACAAGACA | 3660 | TGTCTTGTTGCCACAGTCA |
| 3661 | GACTGTGGCAACAAGACAG | 3662 | CTGTCTTGTTGCCACAGTC |
| 3663 | ACTGTGGCAACAAGACAGT | 3664 | ACTGTCTTGTTGCCACAGT |
| 3665 | CTGTGGCAACAAGACAGTC | 3666 | GACTGTCTTGTTGCCACAG |
| 3667 | TGTGGCAACAAGACAGTCA | 3668 | TGACTGTCTTGTTGCCACA |
| 3669 | GTGGCAACAAGACAGTCAC | 3670 | GTGACTGTCTTGTTGCCAC |
| 3671 | TGGCAACAAGACAGTCACA | 3672 | TGTGACTGTCTTGTTGCCA |
| 3673 | GGCAACAAGACAGTCACAC | 3674 | GTGTGACTGTCTTGTTGCC |
| 3675 | GCAACAAGACAGTCACACC | 3676 | GGTGTGACTGTCTTGTTGC |
| 3677 | CAACAAGACAGTCACACCC | 3678 | GGGTGTGACTGTCTTGTTG |
| 3679 | AACAAGACAGTCACACCCG | 3680 | CGGGTGTGACTGTCTTGTT |
| 3681 | ACAAGACAGTCACACCCGT | 3682 | ACGGGTGTGACTGTCTTGT |
| 3683 | CAAGACAGTCACACCCGTC | 3684 | GACGGGTGTGACTGTCTTG |
| 3685 | AAGACAGTCACACCCGTCA | 3686 | TGACGGGTGTGACTGTCTT |
| 3687 | AGACAGTCACACCCGTCAG | 3688 | CTGACGGGTGTGACTGTCT |
| 3689 | GACAGTCACACCCGTCAGT | 3690 | ACTGACGGGTGTGACTGTC |
| 3691 | ACAGTCACACCCGTCAGTC | 3692 | GACTGACGGGTGTGACTGT |
| 3693 | CAGTCACACCCGTCAGTCC | 3694 | GGACTGACGGGTGTGACTG |
| 3695 | AGTCACACCCGTCAGTCCC | 3696 | GGGACTGACGGGTGTGACT |
| 3697 | GTCACACCCGTCAGTCCCC | 3698 | GGGGACTGACGGGTGTGAC |
| 3699 | TCACACCCGTCAGTCCCCT | 3700 | AGGGGACTGACGGGTGTGA |
| 3701 | CACACCCGTCAGTCCCCTG | 3702 | CAGGGGACTGACGGGTGTG |
| 3703 | ACACCCGTCAGTCCCCTGG | 3704 | CCAGGGGACTGACGGGTGT |
| 3705 | CACCCGTCAGTCCCCTGGG | 3706 | CCCAGGGGACTGACGGGTG |
| 3707 | ACCCGTCAGTCCCCTGGGG | 3708 | CCCCAGGGGACTGACGGGT |
| 3709 | CCCGTCAGTCCCCTGGGGA | 3710 | TCCCCAGGGGACTGACGGG |
| 3711 | CCGTCAGTCCCCTGGGGAA | 3712 | TTCCCCAGGGGACTGACGG |
| 3713 | CGTCAGTCCCCTGGGGAAG | 3714 | CTTCCCCAGGGGACTGACG |
| 3715 | GTCAGTCCCCTGGGGAAGA | 3716 | TCTTCCCCAGGGGACTGAC |
| 3717 | TCAGTCCCCTGGGGAAGAA | 3718 | TTCTTCCCCAGGGGACTGA |
| 3719 | CAGTCCCCTGGGGAAGAAA | 3720 | TTTCTTCCCCAGGGGACTG |
| 3721 | AGTCCCCTGGGGAAGAAAC | 3722 | GTTTCTTCCCCAGGGGACT |
| 3723 | GTCCCCTGGGGAAGAAACT | 3724 | AGTTTCTTCCCCAGGGGAC |
| 3725 | TCCCCTGGGGAAGAAACTA | 3726 | TAGTTTCTTCCCCAGGGGA |
| 3727 | CCCCTGGGGAAGAAACTAA | 3728 | TTAGTTTCTTCCCCAGGGG |
| 3729 | CCCTGGGGAAGAAACTAAA | 3730 | TTTAGTTTCTTCCCCAGGG |
| 3731 | CCTGGGGAAGAAACTAAAT | 3732 | ATTTAGTTTCTTCCCCAGG |
| 3733 | CTGGGGAAGAAACTAAATG | 3734 | CATTTAGTTTCTTCCCCAG |
| 3735 | TGGGGAAGAAACTAAATGT | 3736 | ACATTTAGTTTCTTCCCCA |
| 3737 | GGGGAAGAAACTAAATGTC | 3738 | GACATTTAGTTTCTTCCCC |
| 3739 | GGGAAGAAACTAAATGTCA | 3740 | TGACATTTAGTTTCTTCCC |
| 3741 | GGAAGAAACTAAATGTCAC | 3742 | GTGACATTTAGTTTCTTCC |
| 3743 | GAAGAAACTAAATGTCACA | 3744 | TGTGACATTTAGTTTCTTC |
| 3745 | AAGAAACTAAATGTCACAA | 3746 | TTGTGACATTTAGTTTCTT |
| 3747 | AGAAACTAAATGTCACAAT | 3748 | ATTGTGACATTTAGTTTCT |
| 3749 | GAAACTAAATGTCACAATG | 3750 | CATTGTGACATTTAGTTTC |
| 3751 | AAACTAAATGTCACAATGG | 3752 | CCATTGTGACATTTAGTTT |
| 3753 | AACTAAATGTCACAATGGC | 3754 | GCCATTGTGACATTTAGTT |
| 3755 | ACTAAATGTCACAATGGCC | 3756 | GGCCATTGTGACATTTAGT |
| 3757 | CTAAATGTCACAATGGCCT | 3758 | AGGCCATTGTGACATTTAG |
| 3759 | TAAATGTCACAATGGCCTG | 3760 | CAGGCCATTGTGACATTTA |
| 3761 | AAATGTCACAATGGCCTGG | 3762 | CCAGGCCATTGTGACATTT |
| 3763 | AATGTCACAATGGCCTGGA | 3764 | TCCAGGCCATTGTGACATT |
| 3765 | ATGTCACAATGGCCTGGAA | 3766 | TTCCAGGCCATTGTGACAT |
| 3767 | TGTCACAATGGCCTGGAAA | 3768 | TTTCCAGGCCATTGTGACA |
| 3769 | GTCACAATGGCCTGGAAAG | 3770 | CTTTCCAGGCCATTGTGAC |
| 3771 | TCACAATGGCCTGGAAAGC | 3772 | GCTTTCCAGGCCATTGTGA |
| 3773 | CACAATGGCCTGGAAAGCA | 3774 | TGCTTTCCAGGCCATTGTG |
| 3775 | ACAATGGCCTGGAAAGCAC | 3776 | GTGCTTTCCAGGCCATTGT |
| 3777 | CAATGGCCTGGAAAGCACA | 3778 | TGTGCTTTCCAGGCCATTG |
| 3779 | AATGGCCTGGAAAGCACAG | 3780 | CTGTGCTTTCCAGGCCATT |
| 3781 | ATGGCCTGGAAAGCACAGA | 3782 | TCTGTGCTTTCCAGGCCAT |
| 3783 | TGGCCTGGAAAGCACAGAA | 3784 | TTCTGTGCTTTCCAGGCCA |
| 3785 | GGCCTGGAAAGCACAGAAC | 3786 | GTTCTGTGCTTTCCAGGCC |
| 3787 | GCCTGGAAAGCACAGAACC | 3788 | GGTTCTGTGCTTTCCAGGC |
| 3789 | CCTGGAAAGCACAGAACCC | 3790 | GGGTTCTGTGCTTTCCAGG |
| 3791 | CTGGAAAGCACAGAACCCA | 3792 | TGGGTTCTGTGCTTTCCAG |
| 3793 | TGGAAAGCACAGAACCCAG | 3794 | CTGGGTTCTGTGCTTTCCA |
| 3795 | GGAAAGCACAGAACCCAGT | 3796 | ACTGGGTTCTGTGCTTTCC |
| 3797 | GAAAGCACAGAACCCAGTA | 3798 | TACTGGGTTCTGTGCTTTC |
| 3799 | AAAGCACAGAACCCAGTAC | 3800 | GTACTGGGTTCTGTGCTTT |
| 3801 | AAGCACAGAACCCAGTACT | 3802 | AGTACTGGGTTCTGTGCTT |
| 3803 | AGCACAGAACCCAGTACTG | 3804 | CAGTACTGGGTTCTGTGCT |
| 3805 | GCACAGAACCCAGTACTGA | 3806 | TCAGTACTGGGTTCTGTGC |
| 3807 | CACAGAACCCAGTACTGAG | 3808 | CTCAGTACTGGGTTCTGTG |
| 3809 | ACAGAACCCAGTACTGAGA | 3810 | TCTCAGTACTGGGTTCTGT |
| 3811 | CAGAACCCAGTACTGAGAG | 3812 | CTCTCAGTACTGGGTTCTG |
| 3813 | AGAACCCAGTACTGAGAGA | 3814 | TCTCTCAGTACTGGGTTCT |
| 3815 | GAACCCAGTACTGAGAGAG | 3816 | CTCTCTCAGTACTGGGTTC |
| 3817 | AACCCAGTACTGAGAGAGG | 3818 | CCTCTCTCAGTACTGGGTT |
| 3819 | ACCCAGTACTGAGAGAGGT | 3820 | ACCTCTCTCAGTACTGGGT |
| 3821 | CCCAGTACTGAGAGAGGTG | 3822 | CACCTCTCTCAGTACTGGG |
| 3823 | CCAGTACTGAGAGAGGTGG | 3824 | CCACCTCTCTCAGTACTGG |
| 3825 | CAGTACTGAGAGAGGTGGT | 3826 | ACCACCTCTCTCAGTACTG |
| 3827 | AGTACTGAGAGAGGTGGTG | 3828 | CACCACCTCTCTCAGTACT |
| 3829 | GTACTGAGAGAGGTGGTGG | 3830 | CCACCACCTCTCTCAGTAC |
| 3831 | TACTGAGAGAGGTGGTGGA | 3832 | TCCACCACCTCTCTCAGTA |
| 3833 | ACTGAGAGAGGTGGTGGAC | 3834 | GTCCACCACCTCTCTCAGT |
| 3835 | CTGAGAGAGGTGGTGGACA | 3836 | TGTCCACCACCTCTCTCAG |
| 3837 | TGAGAGAGGTGGTGGACAT | 3838 | ATGTCCACCACCTCTCTCA |
| 3839 | GAGAGAGGTGGTGGACATA | 3840 | TATGTCCACCACCTCTCTC |
| 3841 | AGAGAGGTGGTGGACATAC | 3842 | GTATGTCCACCACCTCTCT |
| 3843 | GAGAGGTGGTGGACATACT | 3844 | AGTATGTCCACCACCTCTC |
| 3845 | AGAGGTGGTGGACATACTT | 3846 | AAGTATGTCCACCACCTCT |
| 3847 | GAGGTGGTGGACATACTTA | 3848 | TAAGTATGTCCACCACCTC |
| 3849 | AGGTGGTGGACATACTTAC | 3850 | GTAAGTATGTCCACCACCT |
| 3851 | GGTGGTGGACATACTTACA | 3852 | TGTAAGTATGTCCACCACC |
| 3853 | GTGGTGGACATACTTACAG | 3854 | CTGTAAGTATGTCCACCAC |
| 3855 | TGGTGGACATACTTACAGA | 3856 | TCTGTAAGTATGTCCACCA |
| 3857 | GGTGGACATACTTACAGAG | 3858 | CTCTGTAAGTATGTCCACC |
| 3859 | GTGGACATACTTACAGAGC | 3860 | GCTCTGTAAGTATGTCCAC |
| 3861 | TGGACATACTTACAGAGCA | 3862 | TGCTCTGTAAGTATGTCCA |
| 3863 | GGACATACTTACAGAGCAA | 3864 | TTGCTCTGTAAGTATGTCC |
| 3865 | GACATACTTACAGAGCAAC | 3866 | GTTGCTCTGTAAGTATGTC |
| 3867 | ACATACTTACAGAGCAACT | 3868 | AGTTGCTCTGTAAGTATGT |
| 3869 | CATACTTACAGAGCAACTG | 3870 | CAGTTGCTCTGTAAGTATG |
| 3871 | ATACTTACAGAGCAACTGC | 3872 | GCAGTTGCTCTGTAAGTAT |
| 3873 | TACTTACAGAGCAACTGCT | 3874 | AGCAGTTGCTCTGTAAGTA |
| 3875 | ACTTACAGAGCAACTGCTT | 3876 | AAGCAGTTGCTCTGTAAGT |
| 3877 | CTTACAGAGCAACTGCTTG | 3878 | CAAGCAGTTGCTCTGTAAG |
| 3879 | TTACAGAGCAACTGCTTGA | 3880 | TCAAGCAGTTGCTCTGTAA |
| 3881 | TACAGAGCAACTGCTTGAC | 3882 | GTCAAGCAGTTGCTCTGTA |
| 3883 | ACAGAGCAACTGCTTGACA | 3884 | TGTCAAGCAGTTGCTCTGT |
| 3885 | CAGAGCAACTGCTTGACAT | 3886 | ATGTCAAGCAGTTGCTCTG |
| 3887 | AGAGCAACTGCTTGACATT | 3888 | AATGTCAAGCAGTTGCTCT |
| 3889 | GAGCAACTGCTTGACATTC | 3890 | GAATGTCAAGCAGTTGCTC |
| 3891 | AGCAACTGCTTGACATTCA | 3892 | TGAATGTCAAGCAGTTGCT |
| 3893 | GCAACTGCTTGACATTCAG | 3894 | CTGAATGTCAAGCAGTTGC |
| 3895 | CAACTGCTTGACATTCAGC | 3896 | GCTGAATGTCAAGCAGTTG |
| 3897 | AACTGCTTGACATTCAGCT | 3898 | AGCTGAATGTCAAGCAGTT |
| 3899 | ACTGCTTGACATTCAGCTG | 3900 | CAGCTGAATGTCAAGCAGT |
| 3901 | CTGCTTGACATTCAGCTGG | 3902 | CCAGCTGAATGTCAAGCAG |
| 3903 | TGCTTGACATTCAGCTGGA | 3904 | TCCAGCTGAATGTCAAGCA |
| 3905 | GCTTGACATTCAGCTGGAG | 3906 | CTCCAGCTGAATGTCAAGC |
| 3907 | CTTGACATTCAGCTGGAGA | 3908 | TCTCCAGCTGAATGTCAAG |
| 3909 | TTGACATTCAGCTGGAGAA | 3910 | TTCTCCAGCTGAATGTCAA |
| 3911 | TGACATTCAGCTGGAGAAT | 3912 | ATTCTCCAGCTGAATGTCA |
| 3913 | GACATTCAGCTGGAGAATT | 3914 | AATTCTCCAGCTGAATGTC |
| 3915 | ACATTCAGCTGGAGAATTA | 3916 | TAATTCTCCAGCTGAATGT |
| 3917 | CATTCAGCTGGAGAATTAC | 3918 | GTAATTCTCCAGCTGAATG |
| 3919 | ATTCAGCTGGAGAATTACA | 3920 | TGTAATTCTCCAGCTGAAT |
| 3921 | TTCAGCTGGAGAATTACAC | 3922 | GTGTAATTCTCCAGCTGAA |
| 3923 | TCAGCTGGAGAATTACACA | 3924 | TGTGTAATTCTCCAGCTGA |
| 3925 | CAGCTGGAGAATTACACAC | 3926 | GTGTGTAATTCTCCAGCTG |
| 3927 | AGCTGGAGAATTACACACC | 3928 | GGTGTGTAATTCTCCAGCT |
| 3929 | GCTGGAGAATTACACACCC | 3930 | GGGTGTGTAATTCTCCAGC |
| 3931 | CTGGAGAATTACACACCCA | 3932 | TGGGTGTGTAATTCTCCAG |
| 3933 | TGGAGAATTACACACCCAA | 3934 | TTGGGTGTGTAATTCTCCA |
| 3935 | GGAGAATTACACACCCAAG | 3936 | CTTGGGTGTGTAATTCTCC |
| 3937 | GAGAATTACACACCCAAGG | 3938 | CCTTGGGTGTGTAATTCTC |
| 3939 | AGAATTACACACCCAAGGA | 3940 | TCCTTGGGTGTGTAATTCT |
| 3941 | GAATTACACACCCAAGGAA | 3942 | TTCCTTGGGTGTGTAATTC |
| 3943 | AATTACACACCCAAGGAAC | 3944 | GTTCCTTGGGTGTGTAATT |
| 3945 | ATTACACACCCAAGGAACC | 3946 | GGTTCCTTGGGTGTGTAAT |
| 3947 | TTACACACCCAAGGAACCC | 3948 | GGGTTCCTTGGGTGTGTAA |
| 3949 | TACACACCCAAGGAACCCC | 3950 | GGGGTTCCTTGGGTGTGTA |
| 3951 | ACACACCCAAGGAACCCCT | 3952 | AGGGGTTCCTTGGGTGTGT |
| 3953 | CACACCCAAGGAACCCCTC | 3954 | GAGGGGTTCCTTGGGTGTG |
| 3955 | ACACCCAAGGAACCCCTCA | 3956 | TGAGGGGTTCCTTGGGTGT |
| 3957 | CACCCAAGGAACCCCTCAC | 3958 | GTGAGGGGTTCCTTGGGTG |
| 3959 | ACCCAAGGAACCCCTCACC | 3960 | GGTGAGGGGTTCCTTGGGT |
| 3961 | CCCAAGGAACCCCTCACCC | 3962 | GGGTGAGGGGTTCCTTGGG |
| 3963 | CCAAGGAACCCCTCACCCT | 3964 | AGGGTGAGGGGTTCCTTGG |
| 3965 | CAAGGAACCCCTCACCCTG | 3966 | CAGGGTGAGGGGTTCCTTG |
| 3967 | AAGGAACCCCTCACCCTGC | 3968 | GCAGGGTGAGGGGTTCCTT |
| 3969 | AGGAACCCCTCACCCTGCA | 3970 | TGCAGGGTGAGGGGTTCCT |
| 3971 | GGAACCCCTCACCCTGCAG | 3972 | CTGCAGGGTGAGGGGTTCC |
| 3973 | GAACCCCTCACCCTGCAGG | 3974 | CCTGCAGGGTGAGGGGTTC |
| 3975 | AACCCCTCACCCTGCAGGC | 3976 | GCCTGCAGGGTGAGGGGTT |
| 3977 | ACCCCTCACCCTGCAGGCA | 3978 | TGCCTGCAGGGTGAGGGGT |
| 3979 | CCCCTCACCCTGCAGGCAA | 3980 | TTGCCTGCAGGGTGAGGGG |
| 3981 | CCCTCACCCTGCAGGCAAG | 3982 | CTTGCCTGCAGGGTGAGGG |
| 3983 | CCTCACCCTGCAGGCAAGG | 3984 | CCTTGCCTGCAGGGTGAGG |
| 3985 | CTCACCCTGCAGGCAAGGA | 3986 | TCCTTGCCTGCAGGGTGAG |
| 3987 | TCACCCTGCAGGCAAGGAT | 3988 | ATCCTTGCCTGCAGGGTGA |
| 3989 | CACCCTGCAGGCAAGGATG | 3990 | CATCCTTGCCTGCAGGGTG |
| 3991 | ACCCTGCAGGCAAGGATGT | 3992 | ACATCCTTGCCTGCAGGGT |
| 3993 | CCCTGCAGGCAAGGATGTC | 3994 | GACATCCTTGCCTGCAGGG |
| 3995 | CCTGCAGGCAAGGATGTCT | 3996 | AGACATCCTTGCCTGCAGG |
| 3997 | CTGCAGGCAAGGATGTCTT | 3998 | AAGACATCCTTGCCTGCAG |
| 3999 | TGCAGGCAAGGATGTCTTG | 4000 | CAAGACATCCTTGCCTGCA |
| 4001 | GCAGGCAAGGATGTCTTGT | 4002 | ACAAGACATCCTTGCCTGC |
| 4003 | CAGGCAAGGATGTCTTGTG | 4004 | CACAAGACATCCTTGCCTG |
| 4005 | AGGCAAGGATGTCTTGTGA | 4006 | TCACAAGACATCCTTGCCT |
| 4007 | GGCAAGGATGTCTTGTGAG | 4008 | CTCACAAGACATCCTTGCC |
| 4009 | GCAAGGATGTCTTGTGAGC | 4010 | GCTCACAAGACATCCTTGC |
| 4011 | CAAGGATGTCTTGTGAGCA | 4012 | TGCTCACAAGACATCCTTG |
| 4013 | AAGGATGTCTTGTGAGCAG | 4014 | CTGCTCACAAGACATCCTT |
| 4015 | AGGATGTCTTGTGAGCAGA | 4016 | TCTGCTCACAAGACATCCT |
| 4017 | GGATGTCTTGTGAGCAGAA | 4018 | TTCTGCTCACAAGACATCC |
| 4019 | GATGTCTTGTGAGCAGAAA | 4020 | TTTCTGCTCACAAGACATC |
| 4021 | ATGTCTTGTGAGCAGAAAG | 4022 | CTTTCTGCTCACAAGACAT |
| 4023 | TGTCTTGTGAGCAGAAAGC | 4024 | GCTTTCTGCTCACAAGACA |
| 4025 | GTCTTGTGAGCAGAAAGCT | 4026 | AGCTTTCTGCTCACAAGAC |
| 4027 | TCTTGTGAGCAGAAAGCTG | 4028 | CAGCTTTCTGCTCACAAGA |
| 4029 | CTTGTGAGCAGAAAGCTGA | 4030 | TCAGCTTTCTGCTCACAAG |
| 4031 | TTGTGAGCAGAAAGCTGAA | 4032 | TTCAGCTTTCTGCTCACAA |
| 4033 | TGTGAGCAGAAAGCTGAAG | 4034 | CTTCAGCTTTCTGCTCACA |
| 4035 | GTGAGCAGAAAGCTGAAGG | 4036 | CCTTCAGCTTTCTGCTCAC |
| 4037 | TGAGCAGAAAGCTGAAGGA | 4038 | TCCTTCAGCTTTCTGCTCA |
| 4039 | GAGCAGAAAGCTGAAGGAC | 4040 | GTCCTTCAGCTTTCTGCTC |
| 4041 | AGCAGAAAGCTGAAGGACA | 4042 | TGTCCTTCAGCTTTCTGCT |
| 4043 | GCAGAAAGCTGAAGGACAC | 4044 | GTGTCCTTCAGCTTTCTGC |
| 4045 | CAGAAAGCTGAAGGACACA | 4046 | TGTGTCCTTCAGCTTTCTG |
| 4047 | AGAAAGCTGAAGGACACAG | 4048 | CTGTGTCCTTCAGCTTTCT |
| 4049 | GAAAGCTGAAGGACACAGC | 4050 | GCTGTGTCCTTCAGCTTTC |
| 4051 | AAAGCTGAAGGACACAGCA | 4052 | TGCTGTGTCCTTCAGCTTT |
| 4053 | AAGCTGAAGGACACAGCAG | 4054 | CTGCTGTGTCCTTCAGCTT |
| 4055 | AGCTGAAGGACACAGCAGT | 4056 | ACTGCTGTGTCCTTCAGCT |
| 4057 | GCTGAAGGACACAGCAGTG | 4058 | CACTGCTGTGTCCTTCAGC |
| 4059 | CTGAAGGACACAGCAGTGG | 4060 | CCACTGCTGTGTCCTTCAG |
| 4061 | TGAAGGACACAGCAGTGGA | 4062 | TCCACTGCTGTGTCCTTCA |
| 4063 | GAAGGACACAGCAGTGGAT | 4064 | ATCCACTGCTGTGTCCTTC |
| 4065 | AAGGACACAGCAGTGGATC | 4066 | GATCCACTGCTGTGTCCTT |
| 4067 | AGGACACAGCAGTGGATCT | 4068 | AGATCCACTGCTGTGTCCT |
| 4069 | GGACACAGCAGTGGATCTT | 4070 | AAGATCCACTGCTGTGTCC |
| 4071 | GACACAGCAGTGGATCTTG | 4072 | CAAGATCCACTGCTGTGTC |
| 4073 | ACACAGCAGTGGATCTTGG | 4074 | CCAAGATCCACTGCTGTGT |
| 4075 | CACAGCAGTGGATCTTGGC | 4076 | GCCAAGATCCACTGCTGTG |
| 4077 | ACAGCAGTGGATCTTGGCA | 4078 | TGCCAAGATCCACTGCTGT |
| 4079 | CAGCAGTGGATCTTGGCAG | 4080 | CTGCCAAGATCCACTGCTG |
| 4081 | AGCAGTGGATCTTGGCAGT | 4082 | ACTGCCAAGATCCACTGCT |
| 4083 | GCAGTGGATCTTGGCAGTT | 4084 | AACTGCCAAGATCCACTGC |
| 4085 | CAGTGGATCTTGGCAGTTC | 4086 | GAACTGCCAAGATCCACTG |
| 4087 | AGTGGATCTTGGCAGTTCA | 4088 | TGAACTGCCAAGATCCACT |
| 4089 | GTGGATCTTGGCAGTTCAG | 4090 | CTGAACTGCCAAGATCCAC |
| 4091 | TGGATCTTGGCAGTTCAGT | 4092 | ACTGAACTGCCAAGATCCA |
| 4093 | GGATCTTGGCAGTTCAGTA | 4094 | TACTGAACTGCCAAGATCC |
| 4095 | GATCTTGGCAGTTCAGTAT | 4096 | ATACTGAACTGCCAAGATC |
| 4097 | ATCTTGGCAGTTCAGTATC | 4098 | GATACTGAACTGCCAAGAT |
| 4099 | TCTTGGCAGTTCAGTATCG | 4100 | CGATACTGAACTGCCAAGA |
| 4101 | CTTGGCAGTTCAGTATCGA | 4102 | TCGATACTGAACTGCCAAG |
| 4103 | TTGGCAGTTCAGTATCGAT | 4104 | ATCGATACTGAACTGCCAA |
| 4105 | TGGCAGTTCAGTATCGATG | 4106 | CATCGATACTGAACTGCCA |
| 4107 | GGCAGTTCAGTATCGATGG | 4108 | CCATCGATACTGAACTGCC |
| 4109 | GCAGTTCAGTATCGATGGA | 4110 | TCCATCGATACTGAACTGC |
| 4111 | CAGTTCAGTATCGATGGAC | 4112 | GTCCATCGATACTGAACTG |
| 4113 | AGTTCAGTATCGATGGACA | 4114 | TGTCCATCGATACTGAACT |
| 4115 | GTTCAGTATCGATGGACAG | 4116 | CTGTCCATCGATACTGAAC |
| 4117 | TTCAGTATCGATGGACAGA | 4118 | TCTGTCCATCGATACTGAA |
| 4119 | TCAGTATCGATGGACAGAC | 4120 | GTCTGTCCATCGATACTGA |
| 4121 | CAGTATCGATGGACAGACC | 4122 | GGTCTGTCCATCGATACTG |
| 4123 | AGTATCGATGGACAGACCT | 4124 | AGGTCTGTCCATCGATACT |
| 4125 | GTATCGATGGACAGACCTT | 4126 | AAGGTCTGTCCATCGATAC |
| 4127 | TATCGATGGACAGACCTTC | 4128 | GAAGGTCTGTCCATCGATA |
| 4129 | ATCGATGGACAGACCTTCC | 4130 | GGAAGGTCTGTCCATCGAT |
| 4131 | TCGATGGACAGACCTTCCT | 4132 | AGGAAGGTCTGTCCATCGA |
| 4133 | CGATGGACAGACCTTCCTA | 4134 | TAGGAAGGTCTGTCCATCG |
| 4135 | GATGGACAGACCTTCCTAC | 4136 | GTAGGAAGGTCTGTCCATC |
| 4137 | ATGGACAGACCTTCCTACT | 4138 | AGTAGGAAGGTCTGTCCAT |
| 4139 | TGGACAGACCTTCCTACTC | 4140 | GAGTAGGAAGGTCTGTCCA |
| 4141 | GGACAGACCTTCCTACTCT | 4142 | AGAGTAGGAAGGTCTGTCC |
| 4143 | GACAGACCTTCCTACTCTT | 4144 | AAGAGTAGGAAGGTCTGTC |
| 4145 | ACAGACCTTCCTACTCTTT | 4146 | AAAGAGTAGGAAGGTCTGT |
| 4147 | CAGACCTTCCTACTCTTTG | 4148 | CAAAGAGTAGGAAGGTCTG |
| 4149 | AGACCTTCCTACTCTTTGA | 4150 | TCAAAGAGTAGGAAGGTCT |
| 4151 | GACCTTCCTACTCTTTGAC | 4152 | GTCAAAGAGTAGGAAGGTC |
| 4153 | ACCTTCCTACTCTTTGACT | 4154 | AGTCAAAGAGTAGGAAGGT |
| 4155 | CCTTCCTACTCTTTGACTC | 4156 | GAGTCAAAGAGTAGGAAGG |
| 4157 | CTTCCTACTCTTTGACTCA | 4158 | TGAGTCAAAGAGTAGGAAG |
| 4159 | TTCCTACTCTTTGACTCAG | 4160 | CTGAGTCAAAGAGTAGGAA |
| 4161 | TCCTACTCTTTGACTCAGA | 4162 | TCTGAGTCAAAGAGTAGGA |
| 4163 | CCTACTCTTTGACTCAGAG | 4164 | CTCTGAGTCAAAGAGTAGG |
| 4165 | CTACTCTTTGACTCAGAGA | 4166 | TCTCTGAGTCAAAGAGTAG |
| 4167 | TACTCTTTGACTCAGAGAA | 4168 | TTCTCTGAGTCAAAGAGTA |
| 4169 | ACTCTTTGACTCAGAGAAG | 4170 | CTTCTCTGAGTCAAAGAGT |
| 4171 | CTCTTTGACTCAGAGAAGA | 4172 | TCTTCTCTGAGTCAAAGAG |
| 4173 | TCTTTGACTCAGAGAAGAG | 4174 | CTCTTCTCTGAGTCAAAGA |
| 4175 | CTTTGACTCAGAGAAGAGA | 4176 | TCTCTTCTCTGAGTCAAAG |
| 4177 | TTTGACTCAGAGAAGAGAA | 4178 | TTCTCTTCTCTGAGTCAAA |
| 4179 | TTGACTCAGAGAAGAGAAT | 4180 | ATTCTCTTCTCTGAGTCAA |
| 4181 | TGACTCAGAGAAGAGAATG | 4182 | CATTCTCTTCTCTGAGTCA |
| 4183 | GACTCAGAGAAGAGAATGT | 4184 | ACATTCTCTTCTCTGAGTC |
| 4185 | ACTCAGAGAAGAGAATGTG | 4186 | CACATTCTCTTCTCTGAGT |
| 4187 | CTCAGAGAAGAGAATGTGG | 4188 | CCACATTCTCTTCTCTGAG |
| 4189 | TCAGAGAAGAGAATGTGGA | 4190 | TCCACATTCTCTTCTCTGA |
| 4191 | CAGAGAAGAGAATGTGGAC | 4192 | GTCCACATTCTCTTCTCTG |
| 4193 | AGAGAAGAGAATGTGGACA | 4194 | TGTCCACATTCTCTTCTCT |
| 4195 | GAGAAGAGAATGTGGACAA | 4196 | TTGTCCACATTCTCTTCTC |
| 4197 | AGAAGAGAATGTGGACAAC | 4198 | GTTGTCCACATTCTCTTCT |
| 4199 | GAAGAGAATGTGGACAACG | 4200 | CGTTGTCCACATTCTCTTC |
| 4201 | AAGAGAATGTGGACAACGG | 4202 | CCGTTGTCCACATTCTCTT |
| 4203 | AGAGAATGTGGACAACGGT | 4204 | ACCGTTGTCCACATTCTCT |
| 4205 | GAGAATGTGGACAACGGTT | 4206 | AACCGTTGTCCACATTCTC |
| 4207 | AGAATGTGGACAACGGTTC | 4208 | GAACCGTTGTCCACATTCT |
| 4209 | GAATGTGGACAACGGTTCA | 4210 | TGAACCGTTGTCCACATTC |
| 4211 | AATGTGGACAACGGTTCAT | 4212 | ATGAACCGTTGTCCACATT |
| 4213 | ATGTGGACAACGGTTCATC | 4214 | GATGAACCGTTGTCCACAT |
| 4215 | TGTGGACAACGGTTCATCC | 4216 | GGATGAACCGTTGTCCACA |
| 4217 | GTGGACAACGGTTCATCCT | 4218 | AGGATGAACCGTTGTCCAC |
| 4219 | TGGACAACGGTTCATCCTG | 4220 | CAGGATGAACCGTTGTCCA |
| 4221 | GGACAACGGTTCATCCTGG | 4222 | CCAGGATGAACCGTTGTCC |
| 4223 | GACAACGGTTCATCCTGGA | 4224 | TCCAGGATGAACCGTTGTC |
| 4225 | ACAACGGTTCATCCTGGAG | 4226 | CTCCAGGATGAACCGTTGT |
| 4227 | CAACGGTTCATCCTGGAGC | 4228 | GCTCCAGGATGAACCGTTG |
| 4229 | AACGGTTCATCCTGGAGCC | 4230 | GGCTCCAGGATGAACCGTT |
| 4231 | ACGGTTCATCCTGGAGCCA | 4232 | TGGCTCCAGGATGAACCGT |
| 4233 | CGGTTCATCCTGGAGCCAG | 4234 | CTGGCTCCAGGATGAACCG |
| 4235 | GGTTCATCCTGGAGCCAGA | 4236 | TCTGGCTCCAGGATGAACC |
| 4237 | GTTCATCCTGGAGCCAGAA | 4238 | TTCTGGCTCCAGGATGAAC |
| 4239 | TTCATCCTGGAGCCAGAAA | 4240 | TTTCTGGCTCCAGGATGAA |
| 4241 | TCATCCTGGAGCCAGAAAG | 4242 | CTTTCTGGCTCCAGGATGA |
| 4243 | CATCCTGGAGCCAGAAAGA | 4244 | TCTTTCTGGCTCCAGGATG |
| 4245 | ATCCTGGAGCCAGAAAGAT | 4246 | ATCTTTCTGGCTCCAGGAT |
| 4247 | TCCTGGAGCCAGAAAGATG | 4248 | CATCTTTCTGGCTCCAGGA |
| 4249 | CCTGGAGCCAGAAAGATGA | 4250 | TCATCTTTCTGGCTCCAGG |
| 4251 | CTGGAGCCAGAAAGATGAA | 4252 | TTCATCTTTCTGGCTCCAG |
| 4253 | TGGAGCCAGAAAGATGAAA | 4254 | TTTCATCTTTCTGGCTCCA |
| 4255 | GGAGCCAGAAAGATGAAAG | 4256 | CTTTCATCTTTCTGGCTCC |
| 4257 | GAGCCAGAAAGATGAAAGA | 4258 | TCTTTCATCTTTCTGGCTC |
| 4259 | AGCCAGAAAGATGAAAGAA | 4260 | TTCTTTCATCTTTCTGGCT |
| 4261 | GCCAGAAAGATGAAAGAAA | 4262 | TTTCTTTCATCTTTCTGGC |
| 4263 | CCAGAAAGATGAAAGAAAA | 4264 | TTTTCTTTCATCTTTCTGG |
| 4265 | CAGAAAGATGAAAGAAAAG | 4266 | CTTTTCTTTCATCTTTCTG |
| 4267 | AGAAAGATGAAAGAAAAGT | 4268 | ACTTTTCTTTCATCTTTCT |
| 4269 | GAAAGATGAAAGAAAAGTG | 4270 | CACTTTTCTTTCATCTTTC |
| 4271 | AAAGATGAAAGAAAAGTGG | 4272 | CCACTTTTCTTTCATCTTT |
| 4273 | AAGATGAAAGAAAAGTGGG | 4274 | CCCACTTTTCTTTCATCTT |
| 4275 | AGATGAAAGAAAAGTGGGA | 4276 | TCCCACTTTTCTTTCATCT |
| 4277 | GATGAAAGAAAAGTGGGAG | 4278 | CTCCCACTTTTCTTTCATC |
| 4279 | ATGAAAGAAAAGTGGGAGA | 4280 | TCTCCCACTTTTCTTTCAT |
| 4281 | TGAAAGAAAAGTGGGAGAA | 4282 | TTCTCCCACTTTTCTTTCA |
| 4283 | GAAAGAAAAGTGGGAGAAT | 4284 | ATTCTCCCACTTTTCTTTC |
| 4285 | AAAGAAAAGTGGGAGAATG | 4286 | CATTCTCCCACTTTTCTTT |
| 4287 | AAGAAAAGTGGGAGAATGA | 4288 | TCATTCTCCCACTTTTCTT |
| 4289 | AGAAAAGTGGGAGAATGAC | 4290 | GTCATTCTCCCACTTTTCT |
| 4291 | GAAAAGTGGGAGAATGACA | 4292 | TGTCATTCTCCCACTTTTC |
| 4293 | AAAAGTGGGAGAATGACAA | 4294 | TTGTCATTCTCCCACTTTT |
| 4295 | AAAGTGGGAGAATGACAAG | 4296 | CTTGTCATTCTCCCACTTT |
| 4297 | AAGTGGGAGAATGACAAGG | 4298 | CCTTGTCATTCTCCCACTT |
| 4299 | AGTGGGAGAATGACAAGGA | 4300 | TCCTTGTCATTCTCCCACT |
| 4301 | GTGGGAGAATGACAAGGAT | 4302 | ATCCTTGTCATTCTCCCAC |
| 4303 | TGGGAGAATGACAAGGATG | 4304 | CATCCTTGTCATTCTCCCA |
| 4305 | GGGAGAATGACAAGGATGT | 4306 | ACATCCTTGTCATTCTCCC |
| 4307 | GGAGAATGACAAGGATGTG | 4308 | CACATCCTTGTCATTCTCC |
| 4309 | GAGAATGACAAGGATGTGG | 4310 | CCACATCCTTGTCATTCTC |
| 4311 | AGAATGACAAGGATGTGGC | 4312 | GCCACATCCTTGTCATTCT |
| 4313 | GAATGACAAGGATGTGGCC | 4314 | GGCCACATCCTTGTCATTC |
| 4315 | AATGACAAGGATGTGGCCA | 4316 | TGGCCACATCCTTGTCATT |
| 4317 | ATGACAAGGATGTGGCCAT | 4318 | ATGGCCACATCCTTGTCAT |
| 4319 | TGACAAGGATGTGGCCATG | 4320 | CATGGCCACATCCTTGTCA |
| 4321 | GACAAGGATGTGGCCATGT | 4322 | ACATGGCCACATCCTTGTC |
| 4323 | ACAAGGATGTGGCCATGTC | 4324 | GACATGGCCACATCCTTGT |
| 4325 | CAAGGATGTGGCCATGTCC | 4326 | GGACATGGCCACATCCTTG |
| 4327 | AAGGATGTGGCCATGTCCT | 4328 | AGGACATGGCCACATCCTT |
| 4329 | AGGATGTGGCCATGTCCTT | 4330 | AAGGACATGGCCACATCCT |
| 4331 | GGATGTGGCCATGTCCTTC | 4332 | GAAGGACATGGCCACATCC |
| 4333 | GATGTGGCCATGTCCTTCC | 4334 | GGAAGGACATGGCCACATC |
| 4335 | ATGTGGCCATGTCCTTCCA | 4336 | TGGAAGGACATGGCCACAT |
| 4337 | TGTGGCCATGTCCTTCCAT | 4338 | ATGGAAGGACATGGCCACA |
| 4339 | GTGGCCATGTCCTTCCATT | 4340 | AATGGAAGGACATGGCCAC |
| 4341 | TGGCCATGTCCTTCCATTA | 4342 | TAATGGAAGGACATGGCCA |
| 4343 | GGCCATGTCCTTCCATTAC | 4344 | GTAATGGAAGGACATGGCC |
| 4345 | GCCATGTCCTTCCATTACA | 4346 | TGTAATGGAAGGACATGGC |
| 4347 | CCATGTCCTTCCATTACAT | 4348 | ATGTAATGGAAGGACATGG |
| 4349 | CATGTCCTTCCATTACATC | 4350 | GATGTAATGGAAGGACATG |
| 4351 | ATGTCCTTCCATTACATCT | 4352 | AGATGTAATGGAAGGACAT |
| 4353 | TGTCCTTCCATTACATCTC | 4354 | GAGATGTAATGGAAGGACA |
| 4355 | GTCCTTCCATTACATCTCA | 4356 | TGAGATGTAATGGAAGGAC |
| 4357 | TCCTTCCATTACATCTCAA | 4358 | TTGAGATGTAATGGAAGGA |
| 4359 | CCTTCCATTACATCTCAAT | 4360 | ATTGAGATGTAATGGAAGG |
| 4361 | CTTCCATTACATCTCAATG | 4362 | CATTGAGATGTAATGGAAG |
| 4363 | TTCCATTACATCTCAATGG | 4364 | CCATTGAGATGTAATGGAA |
| 4365 | TCCATTACATCTCAATGGG | 4366 | CCCATTGAGATGTAATGGA |
| 4367 | CCATTACATCTCAATGGGA | 4368 | TCCCATTGAGATGTAATGG |
| 4369 | CATTACATCTCAATGGGAG | 4370 | CTCCCATTGAGATGTAATG |
| 4371 | ATTACATCTCAATGGGAGA | 4372 | TCTCCCATTGAGATGTAAT |
| 4373 | TTACATCTCAATGGGAGAC | 4374 | GTCTCCCATTGAGATGTAA |
| 4375 | TACATCTCAATGGGAGACT | 4376 | AGTCTCCCATTGAGATGTA |
| 4377 | ACATCTCAATGGGAGACTG | 4378 | CAGTCTCCCATTGAGATGT |
| 4379 | CATCTCAATGGGAGACTGC | 4380 | GCAGTCTCCCATTGAGATG |
| 4381 | ATCTCAATGGGAGACTGCA | 4382 | TGCAGTCTCCCATTGAGAT |
| 4383 | TCTCAATGGGAGACTGCAT | 4384 | ATGCAGTCTCCCATTGAGA |
| 4385 | CTCAATGGGAGACTGCATA | 4386 | TATGCAGTCTCCCATTGAG |
| 4387 | TCAATGGGAGACTGCATAG | 4388 | CTATGCAGTCTCCCATTGA |
| 4389 | CAATGGGAGACTGCATAGG | 4390 | CCTATGCAGTCTCCCATTG |
| 4391 | AATGGGAGACTGCATAGGA | 4392 | TCCTATGCAGTCTCCCATT |
| 4393 | ATGGGAGACTGCATAGGAT | 4394 | ATCCTATGCAGTCTCCCAT |
| 4395 | TGGGAGACTGCATAGGATG | 4396 | CATCCTATGCAGTCTCCCA |
| 4397 | GGGAGACTGCATAGGATGG | 4398 | CCATCCTATGCAGTCTCCC |
| 4399 | GGAGACTGCATAGGATGGC | 4400 | GCCATCCTATGCAGTCTCC |
| 4401 | GAGACTGCATAGGATGGCT | 4402 | AGCCATCCTATGCAGTCTC |
| 4403 | AGACTGCATAGGATGGCTT | 4404 | AAGCCATCCTATGCAGTCT |
| 4405 | GACTGCATAGGATGGCTTG | 4406 | CAAGCCATCCTATGCAGTC |
| 4407 | ACTGCATAGGATGGCTTGA | 4408 | TCAAGCCATCCTATGCAGT |
| 4409 | CTGCATAGGATGGCTTGAG | 4410 | CTCAAGCCATCCTATGCAG |
| 4411 | TGCATAGGATGGCTTGAGG | 4412 | CCTCAAGCCATCCTATGCA |
| 4413 | GCATAGGATGGCTTGAGGA | 4414 | TCCTCAAGCCATCCTATGC |
| 4415 | CATAGGATGGCTTGAGGAC | 4416 | GTCCTCAAGCCATCCTATG |
| 4417 | ATAGGATGGCTTGAGGACT | 4418 | AGTCCTCAAGCCATCCTAT |
| 4419 | TAGGATGGCTTGAGGACTT | 4420 | AAGTCCTCAAGCCATCCTA |
| 4421 | AGGATGGCTTGAGGACTTC | 4422 | GAAGTCCTCAAGCCATCCT |
| 4423 | GGATGGCTTGAGGACTTCT | 4424 | AGAAGTCCTCAAGCCATCC |
| 4425 | GATGGCTTGAGGACTTCTT | 4426 | AAGAAGTCCTCAAGCCATC |
| 4427 | ATGGCTTGAGGACTTCTTG | 4428 | CAAGAAGTCCTCAAGCCAT |
| 4429 | TGGCTTGAGGACTTCTTGA | 4430 | TCAAGAAGTCCTCAAGCCA |
| 4431 | GGCTTGAGGACTTCTTGAT | 4432 | ATCAAGAAGTCCTCAAGCC |
| 4433 | GCTTGAGGACTTCTTGATG | 4434 | CATCAAGAAGTCCTCAAGC |
| 4435 | CTTGAGGACTTCTTGATGG | 4436 | CCATCAAGAAGTCCTCAAG |
| 4437 | TTGAGGACTTCTTGATGGG | 4438 | CCCATCAAGAAGTCCTCAA |
| 4439 | TGAGGACTTCTTGATGGGC | 4440 | GCCCATCAAGAAGTCCTCA |
| 4441 | GAGGACTTCTTGATGGGCA | 4442 | TGCCCATCAAGAAGTCCTC |
| 4443 | AGGACTTCTTGATGGGCAT | 4444 | ATGCCCATCAAGAAGTCCT |
| 4445 | GGACTTCTTGATGGGCATG | 4446 | CATGCCCATCAAGAAGTCC |
| 4447 | GACTTCTTGATGGGCATGG | 4448 | CCATGCCCATCAAGAAGTC |
| 4449 | ACTTCTTGATGGGCATGGA | 4450 | TCCATGCCCATCAAGAAGT |
| 4451 | CTTCTTGATGGGCATGGAC | 4452 | GTCCATGCCCATCAAGAAG |
| 4453 | TTCTTGATGGGCATGGACA | 4454 | TGTCCATGCCCATCAAGAA |
| 4455 | TCTTGATGGGCATGGACAG | 4456 | CTGTCCATGCCCATCAAGA |
| 4457 | CTTGATGGGCATGGACAGC | 4458 | GCTGTCCATGCCCATCAAG |
| 4459 | TTGATGGGCATGGACAGCA | 4460 | TGCTGTCCATGCCCATCAA |
| 4461 | TGATGGGCATGGACAGCAC | 4462 | GTGCTGTCCATGCCCATCA |
| 4463 | GATGGGCATGGACAGCACC | 4464 | GGTGCTGTCCATGCCCATC |
| 4465 | ATGGGCATGGACAGCACCC | 4466 | GGGTGCTGTCCATGCCCAT |
| 4467 | TGGGCATGGACAGCACCCT | 4468 | AGGGTGCTGTCCATGCCCA |
| 4469 | GGGCATGGACAGCACCCTG | 4470 | CAGGGTGCTGTCCATGCCC |
| 4471 | GGCATGGACAGCACCCTGG | 4472 | CCAGGGTGCTGTCCATGCC |
| 4473 | GCATGGACAGCACCCTGGA | 4474 | TCCAGGGTGCTGTCCATGC |
| 4475 | CATGGACAGCACCCTGGAG | 4476 | CTCCAGGGTGCTGTCCATG |
| 4477 | ATGGACAGCACCCTGGAGC | 4478 | GCTCCAGGGTGCTGTCCAT |
| 4479 | TGGACAGCACCCTGGAGCC | 4480 | GGCTCCAGGGTGCTGTCCA |
| 4481 | GGACAGCACCCTGGAGCCA | 4482 | TGGCTCCAGGGTGCTGTCC |
| 4483 | GACAGCACCCTGGAGCCAA | 4484 | TTGGCTCCAGGGTGCTGTC |
| 4485 | ACAGCACCCTGGAGCCAAG | 4486 | CTTGGCTCCAGGGTGCTGT |
| 4487 | CAGCACCCTGGAGCCAAGT | 4488 | ACTTGGCTCCAGGGTGCTG |
| 4489 | AGCACCCTGGAGCCAAGTG | 4490 | CACTTGGCTCCAGGGTGCT |
| 4491 | GCACCCTGGAGCCAAGTGC | 4492 | GCACTTGGCTCCAGGGTGC |
| 4493 | CACCCTGGAGCCAAGTGCA | 4494 | TGCACTTGGCTCCAGGGTG |
| 4495 | ACCCTGGAGCCAAGTGCAG | 4496 | CTGCACTTGGCTCCAGGGT |
| 4497 | CCCTGGAGCCAAGTGCAGG | 4498 | CCTGCACTTGGCTCCAGGG |
| 4499 | CCTGGAGCCAAGTGCAGGA | 4500 | TCCTGCACTTGGCTCCAGG |
| 4501 | CTGGAGCCAAGTGCAGGAG | 4502 | CTCCTGCACTTGGCTCCAG |
| 4503 | TGGAGCCAAGTGCAGGAGC | 4504 | GCTCCTGCACTTGGCTCCA |
| 4505 | GGAGCCAAGTGCAGGAGCA | 4506 | TGCTCCTGCACTTGGCTCC |
| 4507 | GAGCCAAGTGCAGGAGCAC | 4508 | GTGCTCCTGCACTTGGCTC |
| 4509 | AGCCAAGTGCAGGAGCACC | 4510 | GGTGCTCCTGCACTTGGCT |
| 4511 | GCCAAGTGCAGGAGCACCA | 4512 | TGGTGCTCCTGCACTTGGC |
| 4513 | CCAAGTGCAGGAGCACCAC | 4514 | GTGGTGCTCCTGCACTTGG |
| 4515 | CAAGTGCAGGAGCACCACT | 4516 | AGTGGTGCTCCTGCACTTG |
| 4517 | AAGTGCAGGAGCACCACTC | 4518 | GAGTGGTGCTCCTGCACTT |
| 4519 | AGTGCAGGAGCACCACTCG | 4520 | CGAGTGGTGCTCCTGCACT |
| 4521 | GTGCAGGAGCACCACTCGC | 4522 | GCGAGTGGTGCTCCTGCAC |
| 4523 | TGCAGGAGCACCACTCGCC | 4524 | GGCGAGTGGTGCTCCTGCA |
| 4525 | GCAGGAGCACCACTCGCCA | 4526 | TGGCGAGTGGTGCTCCTGC |
| 4527 | CAGGAGCACCACTCGCCAT | 4528 | ATGGCGAGTGGTGCTCCTG |
| 4529 | AGGAGCACCACTCGCCATG | 4530 | CATGGCGAGTGGTGCTCCT |
| 4531 | GGAGCACCACTCGCCATGT | 4532 | ACATGGCGAGTGGTGCTCC |
| 4533 | GAGCACCACTCGCCATGTC | 4534 | GACATGGCGAGTGGTGCTC |
| 4535 | AGCACCACTCGCCATGTCC | 4536 | GGACATGGCGAGTGGTGCT |
| 4537 | GCACCACTCGCCATGTCCT | 4538 | AGGACATGGCGAGTGGTGC |
| 4539 | CACCACTCGCCATGTCCTC | 4540 | GAGGACATGGCGAGTGGTG |
| 4541 | ACCACTCGCCATGTCCTCA | 4542 | TGAGGACATGGCGAGTGGT |
| 4543 | CCACTCGCCATGTCCTCAG | 4544 | CTGAGGACATGGCGAGTGG |
| 4545 | CACTCGCCATGTCCTCAGG | 4546 | CCTGAGGACATGGCGAGTG |
| 4547 | ACTCGCCATGTCCTCAGGC | 4548 | GCCTGAGGACATGGCGAGT |
| 4549 | CTCGCCATGTCCTCAGGCA | 4550 | TGCCTGAGGACATGGCGAG |
| 4551 | TCGCCATGTCCTCAGGCAC | 4552 | GTGCCTGAGGACATGGCGA |
| 4553 | CGCCATGTCCTCAGGCACA | 4554 | TGTGCCTGAGGACATGGCG |
| 4555 | GCCATGTCCTCAGGCACAA | 4556 | TTGTGCCTGAGGACATGGC |
| 4557 | CCATGTCCTCAGGCACAAC | 4558 | GTTGTGCCTGAGGACATGG |
| 4559 | CATGTCCTCAGGCACAACC | 4560 | GGTTGTGCCTGAGGACATG |
| 4561 | ATGTCCTCAGGCACAACCC | 4562 | GGGTTGTGCCTGAGGACAT |
| 4563 | TGTCCTCAGGCACAACCCA | 4564 | TGGGTTGTGCCTGAGGACA |
| 4565 | GTCCTCAGGCACAACCCAA | 4566 | TTGGGTTGTGCCTGAGGAC |
| 4567 | TCCTCAGGCACAACCCAAC | 4568 | GTTGGGTTGTGCCTGAGGA |
| 4569 | CCTCAGGCACAACCCAACT | 4570 | AGTTGGGTTGTGCCTGAGG |
| 4571 | CTCAGGCACAACCCAACTC | 4572 | GAGTTGGGTTGTGCCTGAG |
| 4573 | TCAGGCACAACCCAACTCA | 4574 | TGAGTTGGGTTGTGCCTGA |
| 4575 | CAGGCACAACCCAACTCAG | 4576 | CTGAGTTGGGTTGTGCCTG |
| 4577 | AGGCACAACCCAACTCAGG | 4578 | CCTGAGTTGGGTTGTGCCT |
| 4579 | GGCACAACCCAACTCAGGG | 4580 | CCCTGAGTTGGGTTGTGCC |
| 4581 | GCACAACCCAACTCAGGGC | 4582 | GCCCTGAGTTGGGTTGTGC |
| 4583 | CACAACCCAACTCAGGGCC | 4584 | GGCCCTGAGTTGGGTTGTG |
| 4585 | ACAACCCAACTCAGGGCCA | 4586 | TGGCCCTGAGTTGGGTTGT |
| 4587 | CAACCCAACTCAGGGCCAC | 4588 | GTGGCCCTGAGTTGGGTTG |
| 4589 | AACCCAACTCAGGGCCACA | 4590 | TGTGGCCCTGAGTTGGGTT |
| 4591 | ACCCAACTCAGGGCCACAG | 4592 | CTGTGGCCCTGAGTTGGGT |
| 4593 | CCCAACTCAGGGCCACAGC | 4594 | GCTGTGGCCCTGAGTTGGG |
| 4595 | CCAACTCAGGGCCACAGCC | 4596 | GGCTGTGGCCCTGAGTTGG |
| 4597 | CAACTCAGGGCCACAGCCA | 4598 | TGGCTGTGGCCCTGAGTTG |
| 4599 | AACTCAGGGCCACAGCCAC | 4600 | GTGGCTGTGGCCCTGAGTT |
| 4601 | ACTCAGGGCCACAGCCACC | 4602 | GGTGGCTGTGGCCCTGAGT |
| 4603 | CTCAGGGCCACAGCCACCA | 4604 | TGGTGGCTGTGGCCCTGAG |
| 4605 | TCAGGGCCACAGCCACCAC | 4606 | GTGGTGGCTGTGGCCCTGA |
| 4607 | CAGGGCCACAGCCACCACC | 4608 | GGTGGTGGCTGTGGCCCTG |
| 4609 | AGGGCCACAGCCACCACCC | 4610 | GGGTGGTGGCTGTGGCCCT |
| 4611 | GGGCCACAGCCACCACCCT | 4612 | AGGGTGGTGGCTGTGGCCC |
| 4613 | GGCCACAGCCACCACCCTC | 4614 | GAGGGTGGTGGCTGTGGCC |
| 4615 | GCCACAGCCACCACCCTCA | 4616 | TGAGGGTGGTGGCTGTGGC |
| 4617 | CCACAGCCACCACCCTCAT | 4618 | ATGAGGGTGGTGGCTGTGG |
| 4619 | CACAGCCACCACCCTCATC | 4620 | GATGAGGGTGGTGGCTGTG |
| 4621 | ACAGCCACCACCCTCATCC | 4622 | GGATGAGGGTGGTGGCTGT |
| 4623 | CAGCCACCACCCTCATCCT | 4624 | AGGATGAGGGTGGTGGCTG |
| 4625 | AGCCACCACCCTCATCCTT | 4626 | AAGGATGAGGGTGGTGGCT |
| 4627 | GCCACCACCCTCATCCTTT | 4628 | AAAGGATGAGGGTGGTGGC |
| 4629 | CCACCACCCTCATCCTTTG | 4630 | CAAAGGATGAGGGTGGTGG |
| 4631 | CACCACCCTCATCCTTTGC | 4632 | GCAAAGGATGAGGGTGGTG |
| 4633 | ACCACCCTCATCCTTTGCT | 4634 | AGCAAAGGATGAGGGTGGT |
| 4635 | CCACCCTCATCCTTTGCTG | 4636 | CAGCAAAGGATGAGGGTGG |
| 4637 | CACCCTCATCCTTTGCTGC | 4638 | GCAGCAAAGGATGAGGGTG |
| 4639 | ACCCTCATCCTTTGCTGCC | 4640 | GGCAGCAAAGGATGAGGGT |
| 4641 | CCCTCATCCTTTGCTGCCT | 4642 | AGGCAGCAAAGGATGAGGG |
| 4643 | CCTCATCCTTTGCTGCCTC | 4644 | GAGGCAGCAAAGGATGAGG |
| 4645 | CTCATCCTTTGCTGCCTCC | 4646 | GGAGGCAGCAAAGGATGAG |
| 4647 | TCATCCTTTGCTGCCTCCT | 4648 | AGGAGGCAGCAAAGGATGA |
| 4649 | CATCCTTTGCTGCCTCCTC | 4650 | GAGGAGGCAGCAAAGGATG |
| 4651 | ATCCTTTGCTGCCTCCTCA | 4652 | TGAGGAGGCAGCAAAGGAT |
| 4653 | TCCTTTGCTGCCTCCTCAT | 4654 | ATGAGGAGGCAGCAAAGGA |
| 4655 | CCTTTGCTGCCTCCTCATC | 4656 | GATGAGGAGGCAGCAAAGG |
| 4657 | CTTTGCTGCCTCCTCATCA | 4658 | TGATGAGGAGGCAGCAAAG |
| 4659 | TTTGCTGCCTCCTCATCAT | 4660 | ATGATGAGGAGGCAGCAAA |
| 4661 | TTGCTGCCTCCTCATCATC | 4662 | GATGATGAGGAGGCAGCAA |
| 4663 | TGCTGCCTCCTCATCATCC | 4664 | GGATGATGAGGAGGCAGCA |
| 4665 | GCTGCCTCCTCATCATCCT | 4666 | AGGATGATGAGGAGGCAGC |
| 4667 | CTGCCTCCTCATCATCCTC | 4668 | GAGGATGATGAGGAGGCAG |
| 4669 | TGCCTCCTCATCATCCTCC | 4670 | GGAGGATGATGAGGAGGCA |
| 4671 | GCCTCCTCATCATCCTCCC | 4672 | GGGAGGATGATGAGGAGGC |
| 4673 | CCTCCTCATCATCCTCCCC | 4674 | GGGGAGGATGATGAGGAGG |
| 4675 | CTCCTCATCATCCTCCCCT | 4676 | AGGGGAGGATGATGAGGAG |
| 4677 | TCCTCATCATCCTCCCCTG | 4678 | CAGGGGAGGATGATGAGGA |
| 4679 | CCTCATCATCCTCCCCTGC | 4680 | GCAGGGGAGGATGATGAGG |
| 4681 | CTCATCATCCTCCCCTGCT | 4682 | AGCAGGGGAGGATGATGAG |
| 4683 | TCATCATCCTCCCCTGCTT | 4684 | AAGCAGGGGAGGATGATGA |
| 4685 | CATCATCCTCCCCTGCTTC | 4686 | GAAGCAGGGGAGGATGATG |
| 4687 | ATCATCCTCCCCTGCTTCA | 4688 | TGAAGCAGGGGAGGATGAT |
| 4689 | TCATCCTCCCCTGCTTCAT | 4690 | ATGAAGCAGGGGAGGATGA |
| 4691 | CATCCTCCCCTGCTTCATC | 4692 | GATGAAGCAGGGGAGGATG |
| 4693 | ATCCTCCCCTGCTTCATCC | 4694 | GGATGAAGCAGGGGAGGAT |
| 4695 | TCCTCCCCTGCTTCATCCT | 4696 | AGGATGAAGCAGGGGAGGA |
| 4697 | CCTCCCCTGCTTCATCCTC | 4698 | GAGGATGAAGCAGGGGAGG |
| 4699 | CTCCCCTGCTTCATCCTCC | 4700 | GGAGGATGAAGCAGGGGAG |
| 4701 | TCCCCTGCTTCATCCTCCC | 4702 | GGGAGGATGAAGCAGGGGA |
| 4703 | CCCCTGCTTCATCCTCCCT | 4704 | AGGGAGGATGAAGCAGGGG |
| 4705 | CCCTGCTTCATCCTCCCTG | 4706 | CAGGGAGGATGAAGCAGGG |
| 4707 | CCTGCTTCATCCTCCCTGG | 4708 | CCAGGGAGGATGAAGCAGG |
| 4709 | CTGCTTCATCCTCCCTGGC | 4710 | GCCAGGGAGGATGAAGCAG |
| 4711 | TGCTTCATCCTCCCTGGCA | 4712 | TGCCAGGGAGGATGAAGCA |
| 4713 | GCTTCATCCTCCCTGGCAT | 4714 | ATGCCAGGGAGGATGAAGC |
| 4715 | CTTCATCCTCCCTGGCATC | 4716 | GATGCCAGGGAGGATGAAG |
| 4717 | TTCATCCTCCCTGGCATCT | 4718 | AGATGCCAGGGAGGATGAA |
| 4719 | TCATCCTCCCTGGCATCTG | 4720 | CAGATGCCAGGGAGGATGA |

TABLE 12
Human ULBP3 NM_024518

| SEQID NO. | siRNA (19bp) | SEQID NO. | Reverse complement |
|---|---|---|---|
| 4721 | ATGGCAGCGGCCGCCAGCC | 4722 | GGCTGGCGGCCGCTGCCAT |
| 4723 | TGGCAGCGGCCGCCAGCCC | 4724 | GGGCTGGCGGCCGCTGCCA |
| 4725 | GGCAGCGGCCGCCAGCCCC | 4726 | GGGGCTGGCGGCCGCTGCC |
| 4727 | GCAGCGGCCGCCAGCCCCG | 4728 | CGGGGCTGGCGGCCGCTGC |
| 4729 | CAGCGGCCGCCAGCCCCGC | 4730 | GCGGGGCTGGCGGCCGCTG |
| 4731 | AGCGGCCGCCAGCCCCGCG | 4732 | CGCGGGGCTGGCGGCCGCT |
| 4733 | GCGGCCGCCAGCCCCGCGA | 4734 | TCGCGGGGCTGGCGGCCGC |
| 4735 | CGGCCGCCAGCCCCGCGAT | 4736 | ATCGCGGGGCTGGCGGCCG |
| 4737 | GGCCGCCAGCCCCGCGATC | 4738 | GATCGCGGGGCTGGCGGCC |
| 4739 | GCCGCCAGCCCCGCGATCC | 4740 | GGATCGCGGGGCTGGCGGC |
| 4741 | CCGCCAGCCCCGCGATCCT | 4742 | AGGATCGCGGGGCTGGCGG |
| 4743 | CGCCAGCCCCGCGATCCTT | 4744 | AAGGATCGCGGGGCTGGCG |
| 4745 | GCCAGCCCCGCGATCCTTC | 4746 | GAAGGATCGCGGGGCTGGC |
| 4747 | CCAGCCCCGCGATCCTTCC | 4748 | GGAAGGATCGCGGGGCTGG |
| 4749 | CAGCCCCGCGATCCTTCCG | 4750 | CGGAAGGATCGCGGGGCTG |
| 4751 | AGCCCCGCGATCCTTCCGC | 4752 | GCGGAAGGATCGCGGGGCT |
| 4753 | GCCCCGCGATCCTTCCGCG | 4754 | CGCGGAAGGATCGCGGGGC |
| 4755 | CCCCGCGATCCTTCCGCGC | 4756 | GCGCGGAAGGATCGCGGGG |
| 4757 | CCCGCGATCCTTCCGCGCC | 4758 | GGCGCGGAAGGATCGCGGG |
| 4759 | CCGCGATCCTTCCGCGCCT | 4760 | AGGCGCGGAAGGATCGCGG |
| 4761 | CGCGATCCTTCCGCGCCTC | 4762 | GAGGCGCGGAAGGATCGCG |
| 4763 | GCGATCCTTCCGCGCCTCG | 4764 | CGAGGCGCGGAAGGATCGC |
| 4765 | CGATCCTTCCGCGCCTCGC | 4766 | GCGAGGCGCGGAAGGATCG |
| 4767 | GATCCTTCCGCGCCTCGCG | 4768 | CGCGAGGCGCGGAAGGATC |
| 4769 | ATCCTTCCGCGCCTCGCGA | 4770 | TCGCGAGGCGCGGAAGGAT |
| 4771 | TCCTTCCGCGCCTCGCGAT | 4772 | ATCGCGAGGCGCGGAAGGA |
| 4773 | CCTTCCGCGCCTCGCGATT | 4774 | AATCGCGAGGCGCGGAAGG |
| 4775 | CTTCCGCGCCTCGCGATTC | 4776 | GAATCGCGAGGCGCGGAAG |
| 4777 | TTCCGCGCCTCGCGATTCT | 4778 | AGAATCGCGAGGCGCGGAA |
| 4779 | TCCGCGCCTCGCGATTCTT | 4780 | AAGAATCGCGAGGCGCGGA |
| 4781 | CCGCGCCTCGCGATTCTTC | 4782 | GAAGAATCGCGAGGCGCGG |
| 4783 | CGCGCCTCGCGATTCTTCC | 4784 | GGAAGAATCGCGAGGCGCG |
| 4785 | GCGCCTCGCGATTCTTCCG | 4786 | CGGAAGAATCGCGAGGCGC |
| 4787 | CGCCTCGCGATTCTTCCGT | 4788 | ACGGAAGAATCGCGAGGCG |
| 4789 | GCCTCGCGATTCTTCCGTA | 4790 | TACGGAAGAATCGCGAGGC |
| 4791 | CCTCGCGATTCTTCCGTAC | 4792 | GTACGGAAGAATCGCGAGG |
| 4793 | CTCGCGATTCTTCCGTACC | 4794 | GGTACGGAAGAATCGCGAG |
| 4795 | TCGCGATTCTTCCGTACCT | 4796 | AGGTACGGAAGAATCGCGA |
| 4797 | CGCGATTCTTCCGTACCTG | 4798 | CAGGTACGGAAGAATCGCG |
| 4799 | GCGATTCTTCCGTACCTGC | 4800 | GCAGGTACGGAAGAATCGC |
| 4801 | CGATTCTTCCGTACCTGCT | 4802 | AGCAGGTACGGAAGAATCG |
| 4803 | GATTCTTCCGTACCTGCTA | 4804 | TAGCAGGTACGGAAGAATC |
| 4805 | ATTCTTCCGTACCTGCTAT | 4806 | ATAGCAGGTACGGAAGAAT |
| 4807 | TTCTTCCGTACCTGCTATT | 4808 | AATAGCAGGTACGGAAGAA |
| 4809 | TCTTCCGTACCTGCTATTC | 4810 | GAATAGCAGGTACGGAAGA |
| 4811 | CTTCCGTACCTGCTATTCG | 4812 | CGAATAGCAGGTACGGAAG |
| 4813 | TTCCGTACCTGCTATTCGA | 4814 | TCGAATAGCAGGTACGGAA |
| 4815 | TCCGTACCTGCTATTCGAC | 4816 | GTCGAATAGCAGGTACGGA |
| 4817 | CCGTACCTGCTATTCGACT | 4818 | AGTCGAATAGCAGGTACGG |
| 4819 | CGTACCTGCTATTCGACTG | 4820 | CAGTCGAATAGCAGGTACG |
| 4821 | GTACCTGCTATTCGACTGG | 4822 | CCAGTCGAATAGCAGGTAC |
| 4823 | TACCTGCTATTCGACTGGT | 4824 | ACCAGTCGAATAGCAGGTA |
| 4825 | ACCTGCTATTCGACTGGTC | 4826 | GACCAGTCGAATAGCAGGT |
| 4827 | CCTGCTATTCGACTGGTCC | 4828 | GGACCAGTCGAATAGCAGG |
| 4829 | CTGCTATTCGACTGGTCCG | 4830 | CGGACCAGTCGAATAGCAG |
| 4831 | TGCTATTCGACTGGTCCGG | 4832 | CCGGACCAGTCGAATAGCA |
| 4833 | GCTATTCGACTGGTCCGGG | 4834 | CCCGGACCAGTCGAATAGC |
| 4835 | CTATTCGACTGGTCCGGGA | 4836 | TCCCGGACCAGTCGAATAG |
| 4837 | TATTCGACTGGTCCGGGAC | 4838 | GTCCCGGACCAGTCGAATA |
| 4839 | ATTCGACTGGTCCGGGACG | 4840 | CGTCCCGGACCAGTCGAAT |
| 4841 | TTCGACTGGTCCGGGACGG | 4842 | CCGTCCCGGACCAGTCGAA |
| 4843 | TCGACTGGTCCGGGACGGG | 4844 | CCCGTCCCGGACCAGTCGA |
| 4845 | CGACTGGTCCGGGACGGGG | 4846 | CCCCGTCCCGGACCAGTCG |
| 4847 | GACTGGTCCGGGACGGGGC | 4848 | GCCCCGTCCCGGACCAGTC |
| 4849 | ACTGGTCCGGGACGGGGCG | 4850 | CGCCCCGTCCCGGACCAGT |
| 4851 | CTGGTCCGGGACGGGGCGG | 4852 | CCGCCCCGTCCCGGACCAG |
| 4853 | TGGTCCGGGACGGGGCGGG | 4854 | CCCGCCCCGTCCCGGACCA |
| 4855 | GGTCCGGGACGGGGCGGGC | 4856 | GCCCGCCCCGTCCCGGACC |
| 4857 | GTCCGGGACGGGGCGGGCC | 4858 | GGCCCGCCCCGTCCCGGAC |
| 4859 | TCCGGGACGGGGCGGGCCG | 4860 | CGGCCCGCCCCGTCCCGGA |
| 4861 | CCGGGACGGGGCGGGCCGA | 4862 | TCGGCCCGCCCCGTCCCGG |
| 4863 | CGGGACGGGGCGGGCCGAC | 4864 | GTCGGCCCGCCCCGTCCCG |
| 4865 | GGGACGGGGCGGGCCGACG | 4866 | CGTCGGCCCGCCCCGTCCC |
| 4867 | GGACGGGGCGGGCCGACGC | 4868 | GCGTCGGCCCGCCCCGTCC |
| 4869 | GACGGGGCGGGCCGACGCT | 4870 | AGCGTCGGCCCGCCCCGTC |
| 4871 | ACGGGGCGGGCCGACGCTC | 4872 | GAGCGTCGGCCCGCCCCGT |
| 4873 | CGGGGCGGGCCGACGCTCA | 4874 | TGAGCGTCGGCCCGCCCCG |
| 4875 | GGGGCGGGCCGACGCTCAC | 4876 | GTGAGCGTCGGCCCGCCCC |
| 4877 | GGGCGGGCCGACGCTCACT | 4878 | AGTGAGCGTCGGCCCGCCC |
| 4879 | GGCGGGCCGACGCTCACTC | 4880 | GAGTGAGCGTCGGCCCGCC |
| 4881 | GCGGGCCGACGCTCACTCT | 4882 | AGAGTGAGCGTCGGCCCGC |
| 4883 | CGGGCCGACGCTCACTCTC | 4884 | GAGAGTGAGCGTCGGCCCG |
| 4885 | GGGCCGACGCTCACTCTCT | 4886 | AGAGAGTGAGCGTCGGCCC |
| 4887 | GGCCGACGCTCACTCTCTC | 4888 | GAGAGAGTGAGCGTCGGCC |
| 4889 | GCCGACGCTCACTCTCTCT | 4890 | AGAGAGAGTGAGCGTCGGC |
| 4891 | CCGACGCTCACTCTCTCTG | 4892 | CAGAGAGAGTGAGCGTCGG |
| 4893 | CGACGCTCACTCTCTCTGG | 4894 | CCAGAGAGAGTGAGCGTCG |
| 4895 | GACGCTCACTCTCTCTGGT | 4896 | ACCAGAGAGAGTGAGCGTC |
| 4897 | ACGCTCACTCTCTCTGGTA | 4898 | TACCAGAGAGAGTGAGCGT |
| 4899 | CGCTCACTCTCTCTGGTAT | 4900 | ATACCAGAGAGAGTGAGCG |
| 4901 | GCTCACTCTCTCTGGTATA | 4902 | TATACCAGAGAGAGTGAGC |
| 4903 | CTCACTCTCTCTGGTATAA | 4904 | TTATACCAGAGAGAGTGAG |
| 4905 | TCACTCTCTCTGGTATAAC | 4906 | GTTATACCAGAGAGAGTGA |
| 4907 | CACTCTCTCTGGTATAACT | 4908 | AGTTATACCAGAGAGAGTG |
| 4909 | ACTCTCTCTGGTATAACTT | 4910 | AAGTTATACCAGAGAGAGT |
| 4911 | CTCTCTCTGGTATAACTTC | 4912 | GAAGTTATACCAGAGAGAG |
| 4913 | TCTCTCTGGTATAACTTCA | 4914 | TGAAGTTATACCAGAGAGA |
| 4915 | CTCTCTGGTATAACTTCAC | 4916 | GTGAAGTTATACCAGAGAG |
| 4917 | TCTCTGGTATAACTTCACC | 4918 | GGTGAAGTTATACCAGAGA |
| 4919 | CTCTGGTATAACTTCACCA | 4920 | TGGTGAAGTTATACCAGAG |
| 4921 | TCTGGTATAACTTCACCAT | 4922 | ATGGTGAAGTTATACCAGA |
| 4923 | CTGGTATAACTTCACCATC | 4924 | GATGGTGAAGTTATACCAG |
| 4925 | TGGTATAACTTCACCATCA | 4926 | TGATGGTGAAGTTATACCA |
| 4927 | GGTATAACTTCACCATCAT | 4928 | ATGATGGTGAAGTTATACC |
| 4929 | GTATAACTTCACCATCATT | 4930 | AATGATGGTGAAGTTATAC |
| 4931 | TATAACTTCACCATCATTC | 4932 | GAATGATGGTGAAGTTATA |
| 4933 | ATAACTTCACCATCATTCA | 4934 | TGAATGATGGTGAAGTTAT |
| 4935 | TAACTTCACCATCATTCAT | 4936 | ATGAATGATGGTGAAGTTA |
| 4937 | AACTTCACCATCATTCATT | 4938 | AATGAATGATGGTGAAGTT |
| 4939 | ACTTCACCATCATTCATTT | 4940 | AAATGAATGATGGTGAAGT |
| 4941 | CTTCACCATCATTCATTTG | 4942 | CAAATGAATGATGGTGAAG |
| 4943 | TTCACCATCATTCATTTGC | 4944 | GCAAATGAATGATGGTGAA |
| 4945 | TCACCATCATTCATTTGCC | 4946 | GGCAAATGAATGATGGTGA |
| 4947 | CACCATCATTCATTTGCCC | 4948 | GGGCAAATGAATGATGGTG |
| 4949 | ACCATCATTCATTTGCCCA | 4950 | TGGGCAAATGAATGATGGT |
| 4951 | CCATCATTCATTTGCCCAG | 4952 | CTGGGCAAATGAATGATGG |
| 4953 | CATCATTCATTTGCCCAGA | 4954 | TCTGGGCAAATGAATGATG |
| 4955 | ATCATTCATTTGCCCAGAC | 4956 | GTCTGGGCAAATGAATGAT |
| 4957 | TCATTCATTTGCCCAGACA | 4958 | TGTCTGGGCAAATGAATGA |
| 4959 | CATTCATTTGCCCAGACAT | 4960 | ATGTCTGGGCAAATGAATG |
| 4961 | ATTCATTTGCCCAGACATG | 4962 | CATGTCTGGGCAAATGAAT |
| 4963 | TTCATTTGCCCAGACATGG | 4964 | CCATGTCTGGGCAAATGAA |
| 4965 | TCATTTGCCCAGACATGGG | 4966 | CCCATGTCTGGGCAAATGA |
| 4967 | CATTTGCCCAGACATGGGC | 4968 | GCCCATGTCTGGGCAAATG |
| 4969 | ATTTGCCCAGACATGGGCA | 4970 | TGCCCATGTCTGGGCAAAT |
| 4971 | TTTGCCCAGACATGGGCAA | 4972 | TTGCCCATGTCTGGGCAAA |
| 4973 | TTGCCCAGACATGGGCAAC | 4974 | GTTGCCCATGTCTGGGCAA |
| 4975 | TGCCCAGACATGGGCAACA | 4976 | TGTTGCCCATGTCTGGGCA |
| 4977 | GCCCAGACATGGGCAACAG | 4978 | CTGTTGCCCATGTCTGGGC |
| 4979 | CCCAGACATGGGCAACAGT | 4980 | ACTGTTGCCCATGTCTGGG |
| 4981 | CCAGACATGGGCAACAGTG | 4982 | CACTGTTGCCCATGTCTGG |
| 4983 | CAGACATGGGCAACAGTGG | 4984 | CCACTGTTGCCCATGTCTG |
| 4985 | AGACATGGGCAACAGTGGT | 4986 | ACCACTGTTGCCCATGTCT |
| 4987 | GACATGGGCAACAGTGGTG | 4988 | CACCACTGTTGCCCATGTC |
| 4989 | ACATGGGCAACAGTGGTGT | 4990 | ACACCACTGTTGCCCATGT |
| 4991 | CATGGGCAACAGTGGTGTG | 4992 | CACACCACTGTTGCCCATG |
| 4993 | ATGGGCAACAGTGGTGTGA | 4994 | TCACACCACTGTTGCCCAT |
| 4995 | TGGGCAACAGTGGTGTGAG | 4996 | CTCACACCACTGTTGCCCA |
| 4997 | GGGCAACAGTGGTGTGAGG | 4998 | CCTCACACCACTGTTGCCC |
| 4999 | GGCAACAGTGGTGTGAGGT | 5000 | ACCTCACACCACTGTTGCC |
| 5001 | GCAACAGTGGTGTGAGGTC | 5002 | GACCTCACACCACTGTTGC |
| 5003 | CAACAGTGGTGTGAGGTCC | 5004 | GGACCTCACACCACTGTTG |
| 5005 | AACAGTGGTGTGAGGTCCA | 5006 | TGGACCTCACACCACTGTT |
| 5007 | ACAGTGGTGTGAGGTCCAG | 5008 | CTGGACCTCACACCACTGT |
| 5009 | CAGTGGTGTGAGGTCCAGA | 5010 | TCTGGACCTCACACCACTG |
| 5011 | AGTGGTGTGAGGTCCAGAG | 5012 | CTCTGGACCTCACACCACT |
| 5013 | GTGGTGTGAGGTCCAGAGC | 5014 | GCTCTGGACCTCACACCAC |
| 5015 | TGGTGTGAGGTCCAGAGCC | 5016 | GGCTCTGGACCTCACACCA |
| 5017 | GGTGTGAGGTCCAGAGCCA | 5018 | TGGCTCTGGACCTCACACC |
| 5019 | GTGTGAGGTCCAGAGCCAG | 5020 | CTGGCTCTGGACCTCACAC |
| 5021 | TGTGAGGTCCAGAGCCAGG | 5022 | CCTGGCTCTGGACCTCACA |
| 5023 | GTGAGGTCCAGAGCCAGGT | 5024 | ACCTGGCTCTGGACCTCAC |
| 5025 | TGAGGTCCAGAGCCAGGTG | 5026 | CACCTGGCTCTGGACCTCA |
| 5027 | GAGGTCCAGAGCCAGGTGG | 5028 | CCACCTGGCTCTGGACCTC |
| 5029 | AGGTCCAGAGCCAGGTGGA | 5030 | TCCACCTGGCTCTGGACCT |
| 5031 | GGTCCAGAGCCAGGTGGAT | 5032 | ATCCACCTGGCTCTGGACC |
| 5033 | GTCCAGAGCCAGGTGGATC | 5034 | GATCCACCTGGCTCTGGAC |
| 5035 | TCCAGAGCCAGGTGGATCA | 5036 | TGATCCACCTGGCTCTGGA |
| 5037 | CCAGAGCCAGGTGGATCAG | 5038 | CTGATCCACCTGGCTCTGG |
| 5039 | CAGAGCCAGGTGGATCAGA | 5040 | TCTGATCCACCTGGCTCTG |
| 5041 | AGAGCCAGGTGGATCAGAA | 5042 | TTCTGATCCACCTGGCTCT |
| 5043 | GAGCCAGGTGGATCAGAAG | 5044 | CTTCTGATCCACCTGGCTC |
| 5045 | AGCCAGGTGGATCAGAAGA | 5046 | TCTTCTGATCCACCTGGCT |
| 5047 | GCCAGGTGGATCAGAAGAA | 5048 | TTCTTCTGATCCACCTGGC |
| 5049 | CCAGGTGGATCAGAAGAAT | 5050 | ATTCTTCTGATCCACCTGG |
| 5051 | CAGGTGGATCAGAAGAATT | 5052 | AATTCTTCTGATCCACCTG |
| 5053 | AGGTGGATCAGAAGAATTT | 5054 | AAATTCTTCTGATCCACCT |
| 5055 | GGTGGATCAGAAGAATTTT | 5056 | AAAATTCTTCTGATCCACC |
| 5057 | GTGGATCAGAAGAATTTTC | 5058 | GAAAATTCTTCTGATCCAC |
| 5059 | TGGATCAGAAGAATTTTCT | 5060 | AGAAAATTCTTCTGATCCA |
| 5061 | GGATCAGAAGAATTTTCTC | 5062 | GAGAAAATTCTTCTGATCC |
| 5063 | GATCAGAAGAATTTTCTCT | 5064 | AGAGAAAATTCTTCTGATC |
| 5065 | ATCAGAAGAATTTTCTCTC | 5066 | GAGAGAAAATTCTTCTGAT |
| 5067 | TCAGAAGAATTTTCTCTCC | 5068 | GGAGAGAAAATTCTTCTGA |
| 5069 | CAGAAGAATTTTCTCTCCT | 5070 | AGGAGAGAAAATTCTTCTG |
| 5071 | AGAAGAATTTTCTCTCCTA | 5072 | TAGGAGAGAAAATTCTTCT |
| 5073 | GAAGAATTTTCTCTCCTAT | 5074 | ATAGGAGAGAAAATTCTTC |
| 5075 | AAGAATTTTCTCTCCTATG | 5076 | CATAGGAGAGAAAATTCTT |
| 5077 | AGAATTTTCTCTCCTATGA | 5078 | TCATAGGAGAGAAAATTCT |
| 5079 | GAATTTTCTCTCCTATGAC | 5080 | GTCATAGGAGAGAAAATTC |
| 5081 | AATTTTCTCTCCTATGACT | 5082 | AGTCATAGGAGAGAAAATT |
| 5083 | ATTTTCTCTCCTATGACTG | 5084 | CAGTCATAGGAGAGAAAAT |
| 5085 | TTTTCTCTCCTATGACTGT | 5086 | ACAGTCATAGGAGAGAAAA |
| 5087 | TTTCTCTCCTATGACTGTG | 5088 | CACAGTCATAGGAGAGAAA |
| 5089 | TTCTCTCCTATGACTGTGG | 5090 | CCACAGTCATAGGAGAGAA |
| 5091 | TCTCTCCTATGACTGTGGC | 5092 | GCCACAGTCATAGGAGAGA |
| 5093 | CTCTCCTATGACTGTGGCA | 5094 | TGCCACAGTCATAGGAGAG |
| 5095 | TCTCCTATGACTGTGGCAG | 5096 | CTGCCACAGTCATAGGAGA |
| 5097 | CTCCTATGACTGTGGCAGT | 5098 | ACTGCCACAGTCATAGGAG |
| 5099 | TCCTATGACTGTGGCAGTG | 5100 | CACTGCCACAGTCATAGGA |
| 5101 | CCTATGACTGTGGCAGTGA | 5102 | TCACTGCCACAGTCATAGG |
| 5103 | CTATGACTGTGGCAGTGAC | 5104 | GTCACTGCCACAGTCATAG |
| 5105 | TATGACTGTGGCAGTGACA | 5106 | TGTCACTGCCACAGTCATA |
| 5107 | ATGACTGTGGCAGTGACAA | 5108 | TTGTCACTGCCACAGTCAT |
| 5109 | TGACTGTGGCAGTGACAAG | 5110 | CTTGTCACTGCCACAGTCA |
| 5111 | GACTGTGGCAGTGACAAGG | 5112 | CCTTGTCACTGCCACAGTC |
| 5113 | ACTGTGGCAGTGACAAGGT | 5114 | ACCTTGTCACTGCCACAGT |
| 5115 | CTGTGGCAGTGACAAGGTC | 5116 | GACCTTGTCACTGCCACAG |
| 5117 | TGTGGCAGTGACAAGGTCT | 5118 | AGACCTTGTCACTGCCACA |
| 5119 | GTGGCAGTGACAAGGTCTT | 5120 | AAGACCTTGTCACTGCCAC |
| 5121 | TGGCAGTGACAAGGTCTTA | 5122 | TAAGACCTTGTCACTGCCA |
| 5123 | GGCAGTGACAAGGTCTTAT | 5124 | ATAAGACCTTGTCACTGCC |
| 5125 | GCAGTGACAAGGTCTTATC | 5126 | GATAAGACCTTGTCACTGC |
| 5127 | CAGTGACAAGGTCTTATCT | 5128 | AGATAAGACCTTGTCACTG |
| 5129 | AGTGACAAGGTCTTATCTA | 5130 | TAGATAAGACCTTGTCACT |
| 5131 | GTGACAAGGTCTTATCTAT | 5132 | ATAGATAAGACCTTGTCAC |
| 5133 | TGACAAGGTCTTATCTATG | 5134 | CATAGATAAGACCTTGTCA |
| 5135 | GACAAGGTCTTATCTATGG | 5136 | CCATAGATAAGACCTTGTC |
| 5137 | ACAAGGTCTTATCTATGGG | 5138 | CCCATAGATAAGACCTTGT |
| 5139 | CAAGGTCTTATCTATGGGT | 5140 | ACCCATAGATAAGACCTTG |
| 5141 | AAGGTCTTATCTATGGGTC | 5142 | GACCCATAGATAAGACCTT |
| 5143 | AGGTCTTATCTATGGGTCA | 5144 | TGACCCATAGATAAGACCT |
| 5145 | GGTCTTATCTATGGGTCAC | 5146 | GTGACCCATAGATAAGACC |
| 5147 | GTCTTATCTATGGGTCACC | 5148 | GGTGACCCATAGATAAGAC |
| 5149 | TCTTATCTATGGGTCACCT | 5150 | AGGTGACCCATAGATAAGA |
| 5151 | CTTATCTATGGGTCACCTA | 5152 | TAGGTGACCCATAGATAAG |
| 5153 | TTATCTATGGGTCACCTAG | 5154 | CTAGGTGACCCATAGATAA |
| 5155 | TATCTATGGGTCACCTAGA | 5156 | TCTAGGTGACCCATAGATA |
| 5157 | ATCTATGGGTCACCTAGAA | 5158 | TTCTAGGTGACCCATAGAT |
| 5159 | TCTATGGGTCACCTAGAAG | 5160 | CTTCTAGGTGACCCATAGA |
| 5161 | CTATGGGTCACCTAGAAGA | 5162 | TCTTCTAGGTGACCCATAG |
| 5163 | TATGGGTCACCTAGAAGAG | 5164 | CTCTTCTAGGTGACCCATA |
| 5165 | ATGGGTCACCTAGAAGAGC | 5166 | GCTCTTCTAGGTGACCCAT |
| 5167 | TGGGTCACCTAGAAGAGCA | 5168 | TGCTCTTCTAGGTGACCCA |
| 5169 | GGGTCACCTAGAAGAGCAG | 5170 | CTGCTCTTCTAGGTGACCC |
| 5171 | GGTCACCTAGAAGAGCAGC | 5172 | GCTGCTCTTCTAGGTGACC |
| 5173 | GTCACCTAGAAGAGCAGCT | 5174 | AGCTGCTCTTCTAGGTGAC |
| 5175 | TCACCTAGAAGAGCAGCTG | 5176 | CAGCTGCTCTTCTAGGTGA |
| 5177 | CACCTAGAAGAGCAGCTGT | 5178 | ACAGCTGCTCTTCTAGGTG |
| 5179 | ACCTAGAAGAGCAGCTGTA | 5180 | TACAGCTGCTCTTCTAGGT |
| 5181 | CCTAGAAGAGCAGCTGTAT | 5182 | ATACAGCTGCTCTTCTAGG |
| 5183 | CTAGAAGAGCAGCTGTATG | 5184 | CATACAGCTGCTCTTCTAG |
| 5185 | TAGAAGAGCAGCTGTATGC | 5186 | GCATACAGCTGCTCTTCTA |
| 5187 | AGAAGAGCAGCTGTATGCC | 5188 | GGCATACAGCTGCTCTTCT |
| 5189 | GAAGAGCAGCTGTATGCCA | 5190 | TGGCATACAGCTGCTCTTC |
| 5191 | AAGAGCAGCTGTATGCCAC | 5192 | GTGGCATACAGCTGCTCTT |
| 5193 | AGAGCAGCTGTATGCCACA | 5194 | TGTGGCATACAGCTGCTCT |
| 5195 | GAGCAGCTGTATGCCACAG | 5196 | CTGTGGCATACAGCTGCTC |
| 5197 | AGCAGCTGTATGCCACAGA | 5198 | TCTGTGGCATACAGCTGCT |
| 5199 | GCAGCTGTATGCCACAGAT | 5200 | ATCTGTGGCATACAGCTGC |
| 5201 | CAGCTGTATGCCACAGATG | 5202 | CATCTGTGGCATACAGCTG |
| 5203 | AGCTGTATGCCACAGATGC | 5204 | GCATCTGTGGCATACAGCT |
| 5205 | GCTGTATGCCACAGATGCC | 5206 | GGCATCTGTGGCATACAGC |
| 5207 | CTGTATGCCACAGATGCCT | 5208 | AGGCATCTGTGGCATACAG |
| 5209 | TGTATGCCACAGATGCCTG | 5210 | CAGGCATCTGTGGCATACA |
| 5211 | GTATGCCACAGATGCCTGG | 5212 | CCAGGCATCTGTGGCATAC |
| 5213 | TATGCCACAGATGCCTGGG | 5214 | CCCAGGCATCTGTGGCATA |
| 5215 | ATGCCACAGATGCCTGGGG | 5216 | CCCCAGGCATCTGTGGCAT |
| 5217 | TGCCACAGATGCCTGGGGA | 5218 | TCCCCAGGCATCTGTGGCA |
| 5219 | GCCACAGATGCCTGGGGAA | 5220 | TTCCCCAGGCATCTGTGGC |
| 5221 | CCACAGATGCCTGGGGAAA | 5222 | TTTCCCCAGGCATCTGTGG |
| 5223 | CACAGATGCCTGGGGAAAA | 5224 | TTTTCCCCAGGCATCTGTG |
| 5225 | ACAGATGCCTGGGGAAAAC | 5226 | GTTTTCCCCAGGCATCTGT |
| 5227 | CAGATGCCTGGGGAAAACA | 5228 | TGTTTTCCCCAGGCATCTG |
| 5229 | AGATGCCTGGGGAAAACAA | 5230 | TTGTTTTCCCCAGGCATCT |
| 5231 | GATGCCTGGGGAAAACAAC | 5232 | GTTGTTTTCCCCAGGCATC |
| 5233 | ATGCCTGGGGAAAACAACT | 5234 | AGTTGTTTTCCCCAGGCAT |
| 5235 | TGCCTGGGGAAAACAACTG | 5236 | CAGTTGTTTTCCCCAGGCA |
| 5237 | GCCTGGGGAAAACAACTGG | 5238 | CCAGTTGTTTTCCCCAGGC |
| 5239 | CCTGGGGAAAACAACTGGA | 5240 | TCCAGTTGTTTTCCCCAGG |
| 5241 | CTGGGGAAAACAACTGGAA | 5242 | TTCCAGTTGTTTTCCCCAG |
| 5243 | TGGGGAAAACAACTGGAAA | 5244 | TTTCCAGTTGTTTTCCCCA |
| 5245 | GGGGAAAACAACTGGAAAT | 5246 | ATTTCCAGTTGTTTTCCCC |
| 5247 | GGGAAAACAACTGGAAATG | 5248 | CATTTCCAGTTGTTTTCCC |
| 5249 | GGAAAACAACTGGAAATGC | 5250 | GCATTTCCAGTTGTTTTCC |
| 5251 | GAAAACAACTGGAAATGCT | 5252 | AGCATTTCCAGTTGTTTTC |
| 5253 | AAAACAACTGGAAATGCTG | 5254 | CAGCATTTCCAGTTGTTTT |
| 5255 | AAACAACTGGAAATGCTGA | 5256 | TCAGCATTTCCAGTTGTTT |
| 5257 | AACAACTGGAAATGCTGAG | 5258 | CTCAGCATTTCCAGTTGTT |
| 5259 | ACAACTGGAAATGCTGAGA | 5260 | TCTCAGCATTTCCAGTTGT |
| 5261 | CAACTGGAAATGCTGAGAG | 5262 | CTCTCAGCATTTCCAGTTG |
| 5263 | AACTGGAAATGCTGAGAGA | 5264 | TCTCTCAGCATTTCCAGTT |
| 5265 | ACTGGAAATGCTGAGAGAG | 5266 | CTCTCTCAGCATTTCCAGT |
| 5267 | CTGGAAATGCTGAGAGAGG | 5268 | CCTCTCTCAGCATTTCCAG |
| 5269 | TGGAAATGCTGAGAGAGGT | 5270 | ACCTCTCTCAGCATTTCCA |
| 5271 | GGAAATGCTGAGAGAGGTG | 5272 | CACCTCTCTCAGCATTTCC |
| 5273 | GAAATGCTGAGAGAGGTGG | 5274 | CCACCTCTCTCAGCATTTC |
| 5275 | AAATGCTGAGAGAGGTGGG | 5276 | CCCACCTCTCTCAGCATTT |
| 5277 | AATGCTGAGAGAGGTGGGG | 5278 | CCCCACCTCTCTCAGCATT |
| 5279 | ATGCTGAGAGAGGTGGGGC | 5280 | GCCCCACCTCTCTCAGCAT |
| 5281 | TGCTGAGAGAGGTGGGGCA | 5282 | TGCCCCACCTCTCTCAGCA |
| 5283 | GCTGAGAGAGGTGGGGCAG | 5284 | CTGCCCCACCTCTCTCAGC |
| 5285 | CTGAGAGAGGTGGGGCAGA | 5286 | TCTGCCCCACCTCTCTCAG |
| 5287 | TGAGAGAGGTGGGGCAGAG | 5288 | CTCTGCCCCACCTCTCTCA |
| 5289 | GAGAGAGGTGGGGCAGAGG | 5290 | CCTCTGCCCCACCTCTCTC |
| 5291 | AGAGAGGTGGGGCAGAGGC | 5292 | GCCTCTGCCCCACCTCTCT |
| 5293 | GAGAGGTGGGGCAGAGGCT | 5294 | AGCCTCTGCCCCACCTCTC |
| 5295 | AGAGGTGGGGCAGAGGCTC | 5296 | GAGCCTCTGCCCCACCTCT |
| 5297 | GAGGTGGGGCAGAGGCTCA | 5298 | TGAGCCTCTGCCCCACCTC |
| 5299 | AGGTGGGGCAGAGGCTCAG | 5300 | CTGAGCCTCTGCCCCACCT |
| 5301 | GGTGGGGCAGAGGCTCAGA | 5302 | TCTGAGCCTCTGCCCCACC |
| 5303 | GTGGGGCAGAGGCTCAGAC | 5304 | GTCTGAGCCTCTGCCCCAC |
| 5305 | TGGGGCAGAGGCTCAGACT | 5306 | AGTCTGAGCCTCTGCCCCA |
| 5307 | GGGGCAGAGGCTCAGACTG | 5308 | CAGTCTGAGCCTCTGCCCC |
| 5309 | GGGCAGAGGCTCAGACTGG | 5310 | CCAGTCTGAGCCTCTGCCC |
| 5311 | GGCAGAGGCTCAGACTGGA | 5312 | TCCAGTCTGAGCCTCTGCC |
| 5313 | GCAGAGGCTCAGACTGGAA | 5314 | TTCCAGTCTGAGCCTCTGC |
| 5315 | CAGAGGCTCAGACTGGAAC | 5316 | GTTCCAGTCTGAGCCTCTG |
| 5317 | AGAGGCTCAGACTGGAACT | 5318 | AGTTCCAGTCTGAGCCTCT |
| 5319 | GAGGCTCAGACTGGAACTG | 5320 | CAGTTCCAGTCTGAGCCTC |
| 5321 | AGGCTCAGACTGGAACTGG | 5322 | CCAGTTCCAGTCTGAGCCT |
| 5323 | GGCTCAGACTGGAACTGGC | 5324 | GCCAGTTCCAGTCTGAGCC |
| 5325 | GCTCAGACTGGAACTGGCT | 5326 | AGCCAGTTCCAGTCTGAGC |
| 5327 | CTCAGACTGGAACTGGCTG | 5328 | CAGCCAGTTCCAGTCTGAG |
| 5329 | TCAGACTGGAACTGGCTGA | 5330 | TCAGCCAGTTCCAGTCTGA |
| 5331 | CAGACTGGAACTGGCTGAC | 5332 | GTCAGCCAGTTCCAGTCTG |
| 5333 | AGACTGGAACTGGCTGACA | 5334 | TGTCAGCCAGTTCCAGTCT |
| 5335 | GACTGGAACTGGCTGACAC | 5336 | GTGTCAGCCAGTTCCAGTC |
| 5337 | ACTGGAACTGGCTGACACT | 5338 | AGTGTCAGCCAGTTCCAGT |
| 5339 | CTGGAACTGGCTGACACTG | 5340 | CAGTGTCAGCCAGTTCCAG |
| 5341 | TGGAACTGGCTGACACTGA | 5342 | TCAGTGTCAGCCAGTTCCA |
| 5343 | GGAACTGGCTGACACTGAG | 5344 | CTCAGTGTCAGCCAGTTCC |
| 5345 | GAACTGGCTGACACTGAGC | 5346 | GCTCAGTGTCAGCCAGTTC |
| 5347 | AACTGGCTGACACTGAGCT | 5348 | AGCTCAGTGTCAGCCAGTT |
| 5349 | ACTGGCTGACACTGAGCTG | 5350 | CAGCTCAGTGTCAGCCAGT |
| 5351 | CTGGCTGACACTGAGCTGG | 5352 | CCAGCTCAGTGTCAGCCAG |
| 5353 | TGGCTGACACTGAGCTGGA | 5354 | TCCAGCTCAGTGTCAGCCA |
| 5355 | GGCTGACACTGAGCTGGAG | 5356 | CTCCAGCTCAGTGTCAGCC |
| 5357 | GCTGACACTGAGCTGGAGG | 5358 | CCTCCAGCTCAGTGTCAGC |
| 5359 | CTGACACTGAGCTGGAGGA | 5360 | TCCTCCAGCTCAGTGTCAG |
| 5361 | TGACACTGAGCTGGAGGAT | 5362 | ATCCTCCAGCTCAGTGTCA |
| 5363 | GACACTGAGCTGGAGGATT | 5364 | AATCCTCCAGCTCAGTGTC |
| 5365 | ACACTGAGCTGGAGGATTT | 5366 | AAATCCTCCAGCTCAGTGT |
| 5367 | CACTGAGCTGGAGGATTTC | 5368 | GAAATCCTCCAGCTCAGTG |
| 5369 | ACTGAGCTGGAGGATTTCA | 5370 | TGAAATCCTCCAGCTCAGT |
| 5371 | CTGAGCTGGAGGATTTCAC | 5372 | GTGAAATCCTCCAGCTCAG |
| 5373 | TGAGCTGGAGGATTTCACA | 5374 | TGTGAAATCCTCCAGCTCA |
| 5375 | GAGCTGGAGGATTTCACAC | 5376 | GTGTGAAATCCTCCAGCTC |
| 5377 | AGCTGGAGGATTTCACACC | 5378 | GGTGTGAAATCCTCCAGCT |
| 5379 | GCTGGAGGATTTCACACCC | 5380 | GGGTGTGAAATCCTCCAGC |
| 5381 | CTGGAGGATTTCACACCCA | 5382 | TGGGTGTGAAATCCTCCAG |
| 5383 | TGGAGGATTTCACACCCAG | 5384 | CTGGGTGTGAAATCCTCCA |
| 5385 | GGAGGATTTCACACCCAGT | 5386 | ACTGGGTGTGAAATCCTCC |
| 5387 | GAGGATTTCACACCCAGTG | 5388 | CACTGGGTGTGAAATCCTC |
| 5389 | AGGATTTCACACCCAGTGG | 5390 | CCACTGGGTGTGAAATCCT |
| 5391 | GGATTTCACACCCAGTGGA | 5392 | TCCACTGGGTGTGAAATCC |
| 5393 | GATTTCACACCCAGTGGAC | 5394 | GTCCACTGGGTGTGAAATC |
| 5395 | ATTTCACACCCAGTGGACC | 5396 | GGTCCACTGGGTGTGAAAT |
| 5397 | TTTCACACCCAGTGGACCC | 5398 | GGGTCCACTGGGTGTGAAA |
| 5399 | TTCACACCCAGTGGACCCC | 5400 | GGGGTCCACTGGGTGTGAA |
| 5401 | TCACACCCAGTGGACCCCT | 5402 | AGGGGTCCACTGGGTGTGA |
| 5403 | CACACCCAGTGGACCCCTC | 5404 | GAGGGGTCCACTGGGTGTG |
| 5405 | ACACCCAGTGGACCCCTCA | 5406 | TGAGGGGTCCACTGGGTGT |
| 5407 | CACCCAGTGGACCCCTCAC | 5408 | GTGAGGGGTCCACTGGGTG |
| 5409 | ACCCAGTGGACCCCTCACG | 5410 | CGTGAGGGGTCCACTGGGT |
| 5411 | CCCAGTGGACCCCTCACGC | 5412 | GCGTGAGGGGTCCACTGGG |
| 5413 | CCAGTGGACCCCTCACGCT | 5414 | AGCGTGAGGGGTCCACTGG |
| 5415 | CAGTGGACCCCTCACGCTG | 5416 | CAGCGTGAGGGGTCCACTG |
| 5417 | AGTGGACCCCTCACGCTGC | 5418 | GCAGCGTGAGGGGTCCACT |
| 5419 | GTGGACCCCTCACGCTGCA | 5420 | TGCAGCGTGAGGGGTCCAC |
| 5421 | TGGACCCCTCACGCTGCAG | 5422 | CTGCAGCGTGAGGGGTCCA |
| 5423 | GGACCCCTCACGCTGCAGG | 5424 | CCTGCAGCGTGAGGGGTCC |
| 5425 | GACCCCTCACGCTGCAGGT | 5426 | ACCTGCAGCGTGAGGGGTC |
| 5427 | ACCCCTCACGCTGCAGGTC | 5428 | GACCTGCAGCGTGAGGGGT |
| 5429 | CCCCTCACGCTGCAGGTCA | 5430 | TGACCTGCAGCGTGAGGGG |
| 5431 | CCCTCACGCTGCAGGTCAG | 5432 | CTGACCTGCAGCGTGAGGG |
| 5433 | CCTCACGCTGCAGGTCAGG | 5434 | CCTGACCTGCAGCGTGAGG |
| 5435 | CTCACGCTGCAGGTCAGGA | 5436 | TCCTGACCTGCAGCGTGAG |
| 5437 | TCACGCTGCAGGTCAGGAT | 5438 | ATCCTGACCTGCAGCGTGA |
| 5439 | CACGCTGCAGGTCAGGATG | 5440 | CATCCTGACCTGCAGCGTG |
| 5441 | ACGCTGCAGGTCAGGATGT | 5442 | ACATCCTGACCTGCAGCGT |
| 5443 | CGCTGCAGGTCAGGATGTC | 5444 | GACATCCTGACCTGCAGCG |
| 5445 | GCTGCAGGTCAGGATGTCT | 5446 | AGACATCCTGACCTGCAGC |
| 5447 | CTGCAGGTCAGGATGTCTT | 5448 | AAGACATCCTGACCTGCAG |
| 5449 | TGCAGGTCAGGATGTCTTG | 5450 | CAAGACATCCTGACCTGCA |
| 5451 | GCAGGTCAGGATGTCTTGT | 5452 | ACAAGACATCCTGACCTGC |
| 5453 | CAGGTCAGGATGTCTTGTG | 5454 | CACAAGACATCCTGACCTG |
| 5455 | AGGTCAGGATGTCTTGTGA | 5456 | TCACAAGACATCCTGACCT |
| 5457 | GGTCAGGATGTCTTGTGAG | 5458 | CTCACAAGACATCCTGACC |
| 5459 | GTCAGGATGTCTTGTGAGT | 5460 | ACTCACAAGACATCCTGAC |
| 5461 | TCAGGATGTCTTGTGAGTG | 5462 | CACTCACAAGACATCCTGA |
| 5463 | CAGGATGTCTTGTGAGTGT | 5464 | ACACTCACAAGACATCCTG |
| 5465 | AGGATGTCTTGTGAGTGTG | 5466 | CACACTCACAAGACATCCT |
| 5467 | GGATGTCTTGTGAGTGTGA | 5468 | TCACACTCACAAGACATCC |
| 5469 | GATGTCTTGTGAGTGTGAA | 5470 | TTCACACTCACAAGACATC |
| 5471 | ATGTCTTGTGAGTGTGAAG | 5472 | CTTCACACTCACAAGACAT |
| 5473 | TGTCTTGTGAGTGTGAAGC | 5474 | GCTTCACACTCACAAGACA |
| 5475 | GTCTTGTGAGTGTGAAGCC | 5476 | GGCTTCACACTCACAAGAC |
| 5477 | TCTTGTGAGTGTGAAGCCG | 5478 | CGGCTTCACACTCACAAGA |
| 5479 | CTTGTGAGTGTGAAGCCGA | 5480 | TCGGCTTCACACTCACAAG |
| 5481 | TTGTGAGTGTGAAGCCGAT | 5482 | ATCGGCTTCACACTCACAA |
| 5483 | TGTGAGTGTGAAGCCGATG | 5484 | CATCGGCTTCACACTCACA |
| 5485 | GTGAGTGTGAAGCCGATGG | 5486 | CCATCGGCTTCACACTCAC |
| 5487 | TGAGTGTGAAGCCGATGGA | 5488 | TCCATCGGCTTCACACTCA |
| 5489 | GAGTGTGAAGCCGATGGAT | 5490 | ATCCATCGGCTTCACACTC |
| 5491 | AGTGTGAAGCCGATGGATA | 5492 | TATCCATCGGCTTCACACT |
| 5493 | GTGTGAAGCCGATGGATAC | 5494 | GTATCCATCGGCTTCACAC |
| 5495 | TGTGAAGCCGATGGATACA | 5496 | TGTATCCATCGGCTTCACA |
| 5497 | GTGAAGCCGATGGATACAT | 5498 | ATGTATCCATCGGCTTCAC |
| 5499 | TGAAGCCGATGGATACATC | 5500 | GATGTATCCATCGGCTTCA |
| 5501 | GAAGCCGATGGATACATCC | 5502 | GGATGTATCCATCGGCTTC |
| 5503 | AAGCCGATGGATACATCCG | 5504 | CGGATGTATCCATCGGCTT |
| 5505 | AGCCGATGGATACATCCGT | 5506 | ACGGATGTATCCATCGGCT |
| 5507 | GCCGATGGATACATCCGTG | 5508 | CACGGATGTATCCATCGGC |
| 5509 | CCGATGGATACATCCGTGG | 5510 | CCACGGATGTATCCATCGG |
| 5511 | CGATGGATACATCCGTGGA | 5512 | TCCACGGATGTATCCATCG |
| 5513 | GATGGATACATCCGTGGAT | 5514 | ATCCACGGATGTATCCATC |
| 5515 | ATGGATACATCCGTGGATC | 5516 | GATCCACGGATGTATCCAT |
| 5517 | TGGATACATCCGTGGATCT | 5518 | AGATCCACGGATGTATCCA |
| 5519 | GGATACATCCGTGGATCTT | 5520 | AAGATCCACGGATGTATCC |
| 5521 | GATACATCCGTGGATCTTG | 5522 | CAAGATCCACGGATGTATC |
| 5523 | ATACATCCGTGGATCTTGG | 5524 | CCAAGATCCACGGATGTAT |
| 5525 | TACATCCGTGGATCTTGGC | 5526 | GCCAAGATCCACGGATGTA |
| 5527 | ACATCCGTGGATCTTGGCA | 5528 | TGCCAAGATCCACGGATGT |
| 5529 | CATCCGTGGATCTTGGCAG | 5530 | CTGCCAAGATCCACGGATG |
| 5531 | ATCCGTGGATCTTGGCAGT | 5532 | ACTGCCAAGATCCACGGAT |
| 5533 | TCCGTGGATCTTGGCAGTT | 5534 | AACTGCCAAGATCCACGGA |
| 5535 | CCGTGGATCTTGGCAGTTC | 5536 | GAACTGCCAAGATCCACGG |
| 5537 | CGTGGATCTTGGCAGTTCA | 5538 | TGAACTGCCAAGATCCACG |
| 5539 | GTGGATCTTGGCAGTTCAG | 5540 | CTGAACTGCCAAGATCCAC |
| 5541 | TGGATCTTGGCAGTTCAGC | 5542 | GCTGAACTGCCAAGATCCA |
| 5543 | GGATCTTGGCAGTTCAGCT | 5544 | AGCTGAACTGCCAAGATCC |
| 5545 | GATCTTGGCAGTTCAGCTT | 5546 | AAGCTGAACTGCCAAGATC |
| 5547 | ATCTTGGCAGTTCAGCTTC | 5548 | GAAGCTGAACTGCCAAGAT |
| 5549 | TCTTGGCAGTTCAGCTTCG | 5550 | CGAAGCTGAACTGCCAAGA |
| 5551 | CTTGGCAGTTCAGCTTCGA | 5552 | TCGAAGCTGAACTGCCAAG |
| 5553 | TTGGCAGTTCAGCTTCGAT | 5554 | ATCGAAGCTGAACTGCCAA |
| 5555 | TGGCAGTTCAGCTTCGATG | 5556 | CATCGAAGCTGAACTGCCA |
| 5557 | GGCAGTTCAGCTTCGATGG | 5558 | CCATCGAAGCTGAACTGCC |
| 5559 | GCAGTTCAGCTTCGATGGA | 5560 | TCCATCGAAGCTGAACTGC |
| 5561 | CAGTTCAGCTTCGATGGAC | 5562 | GTCCATCGAAGCTGAACTG |
| 5563 | AGTTCAGCTTCGATGGACG | 5564 | CGTCCATCGAAGCTGAACT |
| 5565 | GTTCAGCTTCGATGGACGG | 5566 | CCGTCCATCGAAGCTGAAC |
| 5567 | TTCAGCTTCGATGGACGGA | 5568 | TCCGTCCATCGAAGCTGAA |
| 5569 | TCAGCTTCGATGGACGGAA | 5570 | TTCCGTCCATCGAAGCTGA |
| 5571 | CAGCTTCGATGGACGGAAG | 5572 | CTTCCGTCCATCGAAGCTG |
| 5573 | AGCTTCGATGGACGGAAGT | 5574 | ACTTCCGTCCATCGAAGCT |
| 5575 | GCTTCGATGGACGGAAGTT | 5576 | AACTTCCGTCCATCGAAGC |
| 5577 | CTTCGATGGACGGAAGTTC | 5578 | GAACTTCCGTCCATCGAAG |
| 5579 | TTCGATGGACGGAAGTTCC | 5580 | GGAACTTCCGTCCATCGAA |
| 5581 | TCGATGGACGGAAGTTCCT | 5582 | AGGAACTTCCGTCCATCGA |
| 5583 | CGATGGACGGAAGTTCCTC | 5584 | GAGGAACTTCCGTCCATCG |
| 5585 | GATGGACGGAAGTTCCTCC | 5586 | GGAGGAACTTCCGTCCATC |
| 5587 | ATGGACGGAAGTTCCTCCT | 5588 | AGGAGGAACTTCCGTCCAT |
| 5589 | TGGACGGAAGTTCCTCCTC | 5590 | GAGGAGGAACTTCCGTCCA |
| 5591 | GGACGGAAGTTCCTCCTCT | 5592 | AGAGGAGGAACTTCCGTCC |
| 5593 | GACGGAAGTTCCTCCTCTT | 5594 | AAGAGGAGGAACTTCCGTC |
| 5595 | ACGGAAGTTCCTCCTCTTT | 5596 | AAAGAGGAGGAACTTCCGT |
| 5597 | CGGAAGTTCCTCCTCTTTG | 5598 | CAAAGAGGAGGAACTTCCG |
| 5599 | GGAAGTTCCTCCTCTTTGA | 5600 | TCAAAGAGGAGGAACTTCC |
| 5601 | GAAGTTCCTCCTCTTTGAC | 5602 | GTCAAAGAGGAGGAACTTC |
| 5603 | AAGTTCCTCCTCTTTGACT | 5604 | AGTCAAAGAGGAGGAACTT |
| 5605 | AGTTCCTCCTCTTTGACTC | 5606 | GAGTCAAAGAGGAGGAACT |
| 5607 | GTTCCTCCTCTTTGACTCA | 5608 | TGAGTCAAAGAGGAGGAAC |
| 5609 | TTCCTCCTCTTTGACTCAA | 5610 | TTGAGTCAAAGAGGAGGAA |
| 5611 | TCCTCCTCTTTGACTCAAA | 5612 | TTTGAGTCAAAGAGGAGGA |
| 5613 | CCTCCTCTTTGACTCAAAC | 5614 | GTTTGAGTCAAAGAGGAGG |
| 5615 | CTCCTCTTTGACTCAAACA | 5616 | TGTTTGAGTCAAAGAGGAG |
| 5617 | TCCTCTTTGACTCAAACAA | 5618 | TTGTTTGAGTCAAAGAGGA |
| 5619 | CCTCTTTGACTCAAACAAC | 5620 | GTTGTTTGAGTCAAAGAGG |
| 5621 | CTCTTTGACTCAAACAACA | 5622 | TGTTGTTTGAGTCAAAGAG |
| 5623 | TCTTTGACTCAAACAACAG | 5624 | CTGTTGTTTGAGTCAAAGA |
| 5625 | CTTTGACTCAAACAACAGA | 5626 | TCTGTTGTTTGAGTCAAAG |
| 5627 | TTTGACTCAAACAACAGAA | 5628 | TTCTGTTGTTTGAGTCAAA |
| 5629 | TTGACTCAAACAACAGAAA | 5630 | TTTCTGTTGTTTGAGTCAA |
| 5631 | TGACTCAAACAACAGAAAG | 5632 | CTTTCTGTTGTTTGAGTCA |
| 5633 | GACTCAAACAACAGAAAGT | 5634 | ACTTTCTGTTGTTTGAGTC |
| 5635 | ACTCAAACAACAGAAAGTG | 5636 | CACTTTCTGTTGTTTGAGT |
| 5637 | CTCAAACAACAGAAAGTGG | 5638 | CCACTTTCTGTTGTTTGAG |
| 5639 | TCAAACAACAGAAAGTGGA | 5640 | TCCACTTTCTGTTGTTTGA |
| 5641 | CAAACAACAGAAAGTGGAC | 5642 | GTCCACTTTCTGTTGTTTG |
| 5643 | AAACAACAGAAAGTGGACA | 5644 | TGTCCACTTTCTGTTGTTT |
| 5645 | AACAACAGAAAGTGGACAG | 5646 | CTGTCCACTTTCTGTTGTT |
| 5647 | ACAACAGAAAGTGGACAGT | 5648 | ACTGTCCACTTTCTGTTGT |
| 5649 | CAACAGAAAGTGGACAGTG | 5650 | CACTGTCCACTTTCTGTTG |
| 5651 | AACAGAAAGTGGACAGTGG | 5652 | CCACTGTCCACTTTCTGTT |
| 5653 | ACAGAAAGTGGACAGTGGT | 5654 | ACCACTGTCCACTTTCTGT |
| 5655 | CAGAAAGTGGACAGTGGTT | 5656 | AACCACTGTCCACTTTCTG |
| 5657 | AGAAAGTGGACAGTGGTTC | 5658 | GAACCACTGTCCACTTTCT |
| 5659 | GAAAGTGGACAGTGGTTCA | 5660 | TGAACCACTGTCCACTTTC |
| 5661 | AAAGTGGACAGTGGTTCAC | 5662 | GTGAACCACTGTCCACTTT |
| 5663 | AAGTGGACAGTGGTTCACG | 5664 | CGTGAACCACTGTCCACTT |
| 5665 | AGTGGACAGTGGTTCACGC | 5666 | GCGTGAACCACTGTCCACT |
| 5667 | GTGGACAGTGGTTCACGCT | 5668 | AGCGTGAACCACTGTCCAC |
| 5669 | TGGACAGTGGTTCACGCTG | 5670 | CAGCGTGAACCACTGTCCA |
| 5671 | GGACAGTGGTTCACGCTGG | 5672 | CCAGCGTGAACCACTGTCC |
| 5673 | GACAGTGGTTCACGCTGGA | 5674 | TCCAGCGTGAACCACTGTC |
| 5675 | ACAGTGGTTCACGCTGGAG | 5676 | CTCCAGCGTGAACCACTGT |
| 5677 | CAGTGGTTCACGCTGGAGC | 5678 | GCTCCAGCGTGAACCACTG |
| 5679 | AGTGGTTCACGCTGGAGCC | 5680 | GGCTCCAGCGTGAACCACT |
| 5681 | GTGGTTCACGCTGGAGCCA | 5682 | TGGCTCCAGCGTGAACCAC |
| 5683 | TGGTTCACGCTGGAGCCAG | 5684 | CTGGCTCCAGCGTGAACCA |
| 5685 | GGTTCACGCTGGAGCCAGG | 5686 | CCTGGCTCCAGCGTGAACC |
| 5687 | GTTCACGCTGGAGCCAGGC | 5688 | GCCTGGCTCCAGCGTGAAC |
| 5689 | TTCACGCTGGAGCCAGGCG | 5690 | CGCCTGGCTCCAGCGTGAA |
| 5691 | TCACGCTGGAGCCAGGCGG | 5692 | CCGCCTGGCTCCAGCGTGA |
| 5693 | CACGCTGGAGCCAGGCGGA | 5694 | TCCGCCTGGCTCCAGCGTG |
| 5695 | ACGCTGGAGCCAGGCGGAT | 5696 | ATCCGCCTGGCTCCAGCGT |
| 5697 | CGCTGGAGCCAGGCGGATG | 5698 | CATCCGCCTGGCTCCAGCG |
| 5699 | GCTGGAGCCAGGCGGATGA | 5700 | TCATCCGCCTGGCTCCAGC |
| 5701 | CTGGAGCCAGGCGGATGAA | 5702 | TTCATCCGCCTGGCTCCAG |
| 5703 | TGGAGCCAGGCGGATGAAA | 5704 | TTTCATCCGCCTGGCTCCA |
| 5705 | GGAGCCAGGCGGATGAAAG | 5706 | CTTTCATCCGCCTGGCTCC |
| 5707 | GAGCCAGGCGGATGAAAGA | 5708 | TCTTTCATCCGCCTGGCTC |
| 5709 | AGCCAGGCGGATGAAAGAG | 5710 | CTCTTTCATCCGCCTGGCT |
| 5711 | GCCAGGCGGATGAAAGAGA | 5712 | TCTCTTTCATCCGCCTGGC |
| 5713 | CCAGGCGGATGAAAGAGAA | 5714 | TTCTCTTTCATCCGCCTGG |
| 5715 | CAGGCGGATGAAAGAGAAG | 5716 | CTTCTCTTTCATCCGCCTG |
| 5717 | AGGCGGATGAAAGAGAAGT | 5718 | ACTTCTCTTTCATCCGCCT |
| 5719 | GGCGGATGAAAGAGAAGTG | 5720 | CACTTCTCTTTCATCCGCC |
| 5721 | GCGGATGAAAGAGAAGTGG | 5722 | CCACTTCTCTTTCATCCGC |
| 5723 | CGGATGAAAGAGAAGTGGG | 5724 | CCCACTTCTCTTTCATCCG |
| 5725 | GGATGAAAGAGAAGTGGGA | 5726 | TCCCACTTCTCTTTCATCC |
| 5727 | GATGAAAGAGAAGTGGGAG | 5728 | CTCCCACTTCTCTTTCATC |
| 5729 | ATGAAAGAGAAGTGGGAGA | 5730 | TCTCCCACTTCTCTTTCAT |
| 5731 | TGAAAGAGAAGTGGGAGAA | 5732 | TTCTCCCACTTCTCTTTCA |
| 5733 | GAAAGAGAAGTGGGAGAAG | 5734 | CTTCTCCCACTTCTCTTTC |
| 5735 | AAAGAGAAGTGGGAGAAGG | 5736 | CCTTCTCCCACTTCTCTTT |
| 5737 | AAGAGAAGTGGGAGAAGGA | 5738 | TCCTTCTCCCACTTCTCTT |
| 5739 | AGAGAAGTGGGAGAAGGAT | 5740 | ATCCTTCTCCCACTTCTCT |
| 5741 | GAGAAGTGGGAGAAGGATA | 5742 | TATCCTTCTCCCACTTCTC |
| 5743 | AGAAGTGGGAGAAGGATAG | 5744 | CTATCCTTCTCCCACTTCT |
| 5745 | GAAGTGGGAGAAGGATAGC | 5746 | GCTATCCTTCTCCCACTTC |
| 5747 | AAGTGGGAGAAGGATAGCG | 5748 | CGCTATCCTTCTCCCACTT |
| 5749 | AGTGGGAGAAGGATAGCGG | 5750 | CCGCTATCCTTCTCCCACT |
| 5751 | GTGGGAGAAGGATAGCGGA | 5752 | TCCGCTATCCTTCTCCCAC |
| 5753 | TGGGAGAAGGATAGCGGAC | 5754 | GTCCGCTATCCTTCTCCCA |
| 5755 | GGGAGAAGGATAGCGGACT | 5756 | AGTCCGCTATCCTTCTCCC |
| 5757 | GGAGAAGGATAGCGGACTG | 5758 | CAGTCCGCTATCCTTCTCC |
| 5759 | GAGAAGGATAGCGGACTGA | 5760 | TCAGTCCGCTATCCTTCTC |
| 5761 | AGAAGGATAGCGGACTGAC | 5762 | GTCAGTCCGCTATCCTTCT |
| 5763 | GAAGGATAGCGGACTGACC | 5764 | GGTCAGTCCGCTATCCTTC |
| 5765 | AAGGATAGCGGACTGACCA | 5766 | TGGTCAGTCCGCTATCCTT |
| 5767 | AGGATAGCGGACTGACCAC | 5768 | GTGGTCAGTCCGCTATCCT |
| 5769 | GGATAGCGGACTGACCACC | 5770 | GGTGGTCAGTCCGCTATCC |
| 5771 | GATAGCGGACTGACCACCT | 5772 | AGGTGGTCAGTCCGCTATC |
| 5773 | ATAGCGGACTGACCACCTT | 5774 | AAGGTGGTCAGTCCGCTAT |
| 5775 | TAGCGGACTGACCACCTTC | 5776 | GAAGGTGGTCAGTCCGCTA |
| 5777 | AGCGGACTGACCACCTTCT | 5778 | AGAAGGTGGTCAGTCCGCT |
| 5779 | GCGGACTGACCACCTTCTT | 5780 | AAGAAGGTGGTCAGTCCGC |
| 5781 | CGGACTGACCACCTTCTTC | 5782 | GAAGAAGGTGGTCAGTCCG |
| 5783 | GGACTGACCACCTTCTTCA | 5784 | TGAAGAAGGTGGTCAGTCC |
| 5785 | GACTGACCACCTTCTTCAA | 5786 | TTGAAGAAGGTGGTCAGTC |
| 5787 | ACTGACCACCTTCTTCAAG | 5788 | CTTGAAGAAGGTGGTCAGT |
| 5789 | CTGACCACCTTCTTCAAGA | 5790 | TCTTGAAGAAGGTGGTCAG |
| 5791 | TGACCACCTTCTTCAAGAT | 5792 | ATCTTGAAGAAGGTGGTCA |
| 5793 | GACCACCTTCTTCAAGATG | 5794 | CATCTTGAAGAAGGTGGTC |
| 5795 | ACCACCTTCTTCAAGATGG | 5796 | CCATCTTGAAGAAGGTGGT |
| 5797 | CCACCTTCTTCAAGATGGT | 5798 | ACCATCTTGAAGAAGGTGG |
| 5799 | CACCTTCTTCAAGATGGTC | 5800 | GACCATCTTGAAGAAGGTG |
| 5801 | ACCTTCTTCAAGATGGTCT | 5802 | AGACCATCTTGAAGAAGGT |
| 5803 | CCTTCTTCAAGATGGTCTC | 5804 | GAGACCATCTTGAAGAAGG |
| 5805 | CTTCTTCAAGATGGTCTCA | 5806 | TGAGACCATCTTGAAGAAG |
| 5807 | TTCTTCAAGATGGTCTCAA | 5808 | TTGAGACCATCTTGAAGAA |
| 5809 | TCTTCAAGATGGTCTCAAT | 5810 | ATTGAGACCATCTTGAAGA |
| 5811 | CTTCAAGATGGTCTCAATG | 5812 | CATTGAGACCATCTTGAAG |
| 5813 | TTCAAGATGGTCTCAATGA | 5814 | TCATTGAGACCATCTTGAA |
| 5815 | TCAAGATGGTCTCAATGAG | 5816 | CTCATTGAGACCATCTTGA |
| 5817 | CAAGATGGTCTCAATGAGA | 5818 | TCTCATTGAGACCATCTTG |
| 5819 | AAGATGGTCTCAATGAGAG | 5820 | CTCTCATTGAGACCATCTT |
| 5821 | AGATGGTCTCAATGAGAGA | 5822 | TCTCTCATTGAGACCATCT |
| 5823 | GATGGTCTCAATGAGAGAC | 5824 | GTCTCTCATTGAGACCATC |
| 5825 | ATGGTCTCAATGAGAGACT | 5826 | AGTCTCTCATTGAGACCAT |
| 5827 | TGGTCTCAATGAGAGACTG | 5828 | CAGTCTCTCATTGAGACCA |
| 5829 | GGTCTCAATGAGAGACTGC | 5830 | GCAGTCTCTCATTGAGACC |
| 5831 | GTCTCAATGAGAGACTGCA | 5832 | TGCAGTCTCTCATTGAGAC |
| 5833 | TCTCAATGAGAGACTGCAA | 5834 | TTGCAGTCTCTCATTGAGA |
| 5835 | CTCAATGAGAGACTGCAAG | 5836 | CTTGCAGTCTCTCATTGAG |
| 5837 | TCAATGAGAGACTGCAAGA | 5838 | TCTTGCAGTCTCTCATTGA |
| 5839 | CAATGAGAGACTGCAAGAG | 5840 | CTCTTGCAGTCTCTCATTG |
| 5841 | AATGAGAGACTGCAAGAGC | 5842 | GCTCTTGCAGTCTCTCATT |
| 5843 | ATGAGAGACTGCAAGAGCT | 5844 | AGCTCTTGCAGTCTCTCAT |
| 5845 | TGAGAGACTGCAAGAGCTG | 5846 | CAGCTCTTGCAGTCTCTCA |
| 5847 | GAGAGACTGCAAGAGCTGG | 5848 | CCAGCTCTTGCAGTCTCTC |
| 5849 | AGAGACTGCAAGAGCTGGC | 5850 | GCCAGCTCTTGCAGTCTCT |
| 5851 | GAGACTGCAAGAGCTGGCT | 5852 | AGCCAGCTCTTGCAGTCTC |
| 5853 | AGACTGCAAGAGCTGGCTT | 5854 | AAGCCAGCTCTTGCAGTCT |
| 5855 | GACTGCAAGAGCTGGCTTA | 5856 | TAAGCCAGCTCTTGCAGTC |
| 5857 | ACTGCAAGAGCTGGCTTAG | 5858 | CTAAGCCAGCTCTTGCAGT |
| 5859 | CTGCAAGAGCTGGCTTAGG | 5860 | CCTAAGCCAGCTCTTGCAG |
| 5861 | TGCAAGAGCTGGCTTAGGG | 5862 | CCCTAAGCCAGCTCTTGCA |
| 5863 | GCAAGAGCTGGCTTAGGGA | 5864 | TCCCTAAGCCAGCTCTTGC |
| 5865 | CAAGAGCTGGCTTAGGGAC | 5866 | GTCCCTAAGCCAGCTCTTG |
| 5867 | AAGAGCTGGCTTAGGGACT | 5868 | AGTCCCTAAGCCAGCTCTT |
| 5869 | AGAGCTGGCTTAGGGACTT | 5870 | AAGTCCCTAAGCCAGCTCT |
| 5871 | GAGCTGGCTTAGGGACTTC | 5872 | GAAGTCCCTAAGCCAGCTC |
| 5873 | AGCTGGCTTAGGGACTTCC | 5874 | GGAAGTCCCTAAGCCAGCT |
| 5875 | GCTGGCTTAGGGACTTCCT | 5876 | AGGAAGTCCCTAAGCCAGC |
| 5877 | CTGGCTTAGGGACTTCCTG | 5878 | CAGGAAGTCCCTAAGCCAG |
| 5879 | TGGCTTAGGGACTTCCTGA | 5880 | TCAGGAAGTCCCTAAGCCA |
| 5881 | GGCTTAGGGACTTCCTGAT | 5882 | ATCAGGAAGTCCCTAAGCC |
| 5883 | GCTTAGGGACTTCCTGATG | 5884 | CATCAGGAAGTCCCTAAGC |
| 5885 | CTTAGGGACTTCCTGATGC | 5886 | GCATCAGGAAGTCCCTAAG |
| 5887 | TTAGGGACTTCCTGATGCA | 5888 | TGCATCAGGAAGTCCCTAA |
| 5889 | TAGGGACTTCCTGATGCAC | 5890 | GTGCATCAGGAAGTCCCTA |
| 5891 | AGGGACTTCCTGATGCACA | 5892 | TGTGCATCAGGAAGTCCCT |
| 5893 | GGGACTTCCTGATGCACAG | 5894 | CTGTGCATCAGGAAGTCCC |
| 5895 | GGACTTCCTGATGCACAGG | 5896 | CCTGTGCATCAGGAAGTCC |
| 5897 | GACTTCCTGATGCACAGGA | 5898 | TCCTGTGCATCAGGAAGTC |
| 5899 | ACTTCCTGATGCACAGGAA | 5900 | TTCCTGTGCATCAGGAAGT |
| 5901 | CTTCCTGATGCACAGGAAG | 5902 | CTTCCTGTGCATCAGGAAG |
| 5903 | TTCCTGATGCACAGGAAGA | 5904 | TCTTCCTGTGCATCAGGAA |
| 5905 | TCCTGATGCACAGGAAGAA | 5906 | TTCTTCCTGTGCATCAGGA |
| 5907 | CCTGATGCACAGGAAGAAG | 5908 | CTTCTTCCTGTGCATCAGG |
| 5909 | CTGATGCACAGGAAGAAGA | 5910 | TCTTCTTCCTGTGCATCAG |
| 5911 | TGATGCACAGGAAGAAGAG | 5912 | CTCTTCTTCCTGTGCATCA |
| 5913 | GATGCACAGGAAGAAGAGG | 5914 | CCTCTTCTTCCTGTGCATC |
| 5915 | ATGCACAGGAAGAAGAGGC | 5916 | GCCTCTTCTTCCTGTGCAT |
| 5917 | TGCACAGGAAGAAGAGGCT | 5918 | AGCCTCTTCTTCCTGTGCA |
| 5919 | GCACAGGAAGAAGAGGCTG | 5920 | CAGCCTCTTCTTCCTGTGC |
| 5921 | CACAGGAAGAAGAGGCTGG | 5922 | CCAGCCTCTTCTTCCTGTG |
| 5923 | ACAGGAAGAAGAGGCTGGA | 5924 | TCCAGCCTCTTCTTCCTGT |
| 5925 | CAGGAAGAAGAGGCTGGAA | 5926 | TTCCAGCCTCTTCTTCCTG |
| 5927 | AGGAAGAAGAGGCTGGAAC | 5928 | GTTCCAGCCTCTTCTTCCT |
| 5929 | GGAAGAAGAGGCTGGAACC | 5930 | GGTTCCAGCCTCTTCTTCC |
| 5931 | GAAGAAGAGGCTGGAACCC | 5932 | GGGTTCCAGCCTCTTCTTC |
| 5933 | AAGAAGAGGCTGGAACCCA | 5934 | TGGGTTCCAGCCTCTTCTT |
| 5935 | AGAAGAGGCTGGAACCCAC | 5936 | GTGGGTTCCAGCCTCTTCT |
| 5937 | GAAGAGGCTGGAACCCACA | 5938 | TGTGGGTTCCAGCCTCTTC |
| 5939 | AAGAGGCTGGAACCCACAG | 5940 | CTGTGGGTTCCAGCCTCTT |
| 5941 | AGAGGCTGGAACCCACAGC | 5942 | GCTGTGGGTTCCAGCCTCT |
| 5943 | GAGGCTGGAACCCACAGCA | 5944 | TGCTGTGGGTTCCAGCCTC |
| 5945 | AGGCTGGAACCCACAGCAC | 5946 | GTGCTGTGGGTTCCAGCCT |
| 5947 | GGCTGGAACCCACAGCACC | 5948 | GGTGCTGTGGGTTCCAGCC |
| 5949 | GCTGGAACCCACAGCACCA | 5950 | TGGTGCTGTGGGTTCCAGC |
| 5951 | CTGGAACCCACAGCACCAC | 5952 | GTGGTGCTGTGGGTTCCAG |
| 5953 | TGGAACCCACAGCACCACC | 5954 | GGTGGTGCTGTGGGTTCCA |
| 5955 | GGAACCCACAGCACCACCC | 5956 | GGGTGGTGCTGTGGGTTCC |
| 5957 | GAACCCACAGCACCACCCA | 5958 | TGGGTGGTGCTGTGGGTTC |
| 5959 | AACCCACAGCACCACCCAC | 5960 | GTGGGTGGTGCTGTGGGTT |
| 5961 | ACCCACAGCACCACCCACC | 5962 | GGTGGGTGGTGCTGTGGGT |
| 5963 | CCCACAGCACCACCCACCA | 5964 | TGGTGGGTGGTGCTGTGGG |
| 5965 | CCACAGCACCACCCACCAT | 5966 | ATGGTGGGTGGTGCTGTGG |
| 5967 | CACAGCACCACCCACCATG | 5968 | CATGGTGGGTGGTGCTGTG |
| 5969 | ACAGCACCACCCACCATGG | 5970 | CCATGGTGGGTGGTGCTGT |
| 5971 | CAGCACCACCCACCATGGC | 5972 | GCCATGGTGGGTGGTGCTG |
| 5973 | AGCACCACCCACCATGGCC | 5974 | GGCCATGGTGGGTGGTGCT |
| 5975 | GCACCACCCACCATGGCCC | 5976 | GGGCCATGGTGGGTGGTGC |
| 5977 | CACCACCCACCATGGCCCC | 5978 | GGGGCCATGGTGGGTGGTG |
| 5979 | ACCACCCACCATGGCCCCA | 5980 | TGGGGCCATGGTGGGTGGT |
| 5981 | CCACCCACCATGGCCCCAG | 5982 | CTGGGGCCATGGTGGGTGG |
| 5983 | CACCCACCATGGCCCCAGG | 5984 | CCTGGGGCCATGGTGGGTG |
| 5985 | ACCCACCATGGCCCCAGGC | 5986 | GCCTGGGGCCATGGTGGGT |
| 5987 | CCCACCATGGCCCCAGGCT | 5988 | AGCCTGGGGCCATGGTGGG |
| 5989 | CCACCATGGCCCCAGGCTT | 5990 | AAGCCTGGGGCCATGGTGG |
| 5991 | CACCATGGCCCCAGGCTTA | 5992 | TAAGCCTGGGGCCATGGTG |
| 5993 | ACCATGGCCCCAGGCTTAG | 5994 | CTAAGCCTGGGGCCATGGT |
| 5995 | CCATGGCCCCAGGCTTAGC | 5996 | GCTAAGCCTGGGGCCATGG |
| 5997 | CATGGCCCCAGGCTTAGCT | 5998 | AGCTAAGCCTGGGGCCATG |
| 5999 | ATGGCCCCAGGCTTAGCTC | 6000 | GAGCTAAGCCTGGGGCCAT |
| 6001 | TGGCCCCAGGCTTAGCTCA | 6002 | TGAGCTAAGCCTGGGGCCA |
| 6003 | GGCCCCAGGCTTAGCTCAA | 6004 | TTGAGCTAAGCCTGGGGCC |
| 6005 | GCCCCAGGCTTAGCTCAAC | 6006 | GTTGAGCTAAGCCTGGGGC |
| 6007 | CCCCAGGCTTAGCTCAACC | 6008 | GGTTGAGCTAAGCCTGGGG |
| 6009 | CCCAGGCTTAGCTCAACCC | 6010 | GGGTTGAGCTAAGCCTGGG |
| 6011 | CCAGGCTTAGCTCAACCCA | 6012 | TGGGTTGAGCTAAGCCTGG |
| 6013 | CAGGCTTAGCTCAACCCAA | 6014 | TTGGGTTGAGCTAAGCCTG |
| 6015 | AGGCTTAGCTCAACCCAAA | 6016 | TTTGGGTTGAGCTAAGCCT |
| 6017 | GGCTTAGCTCAACCCAAAG | 6018 | CTTTGGGTTGAGCTAAGCC |
| 6019 | GCTTAGCTCAACCCAAAGC | 6020 | GCTTTGGGTTGAGCTAAGC |
| 6021 | CTTAGCTCAACCCAAAGCC | 6022 | GGCTTTGGGTTGAGCTAAG |
| 6023 | TTAGCTCAACCCAAAGCCA | 6024 | TGGCTTTGGGTTGAGCTAA |
| 6025 | TAGCTCAACCCAAAGCCAT | 6026 | ATGGCTTTGGGTTGAGCTA |
| 6027 | AGCTCAACCCAAAGCCATA | 6028 | TATGGCTTTGGGTTGAGCT |
| 6029 | GCTCAACCCAAAGCCATAG | 6030 | CTATGGCTTTGGGTTGAGC |
| 6031 | CTCAACCCAAAGCCATAGC | 6032 | GCTATGGCTTTGGGTTGAG |
| 6033 | TCAACCCAAAGCCATAGCC | 6034 | GGCTATGGCTTTGGGTTGA |
| 6035 | CAACCCAAAGCCATAGCCA | 6036 | TGGCTATGGCTTTGGGTTG |
| 6037 | AACCCAAAGCCATAGCCAC | 6038 | GTGGCTATGGCTTTGGGTT |
| 6039 | ACCCAAAGCCATAGCCACC | 6040 | GGTGGCTATGGCTTTGGGT |
| 6041 | CCCAAAGCCATAGCCACCA | 6042 | TGGTGGCTATGGCTTTGGG |
| 6043 | CCAAAGCCATAGCCACCAC | 6044 | GTGGTGGCTATGGCTTTGG |
| 6045 | CAAAGCCATAGCCACCACC | 6046 | GGTGGTGGCTATGGCTTTG |
| 6047 | AAAGCCATAGCCACCACCC | 6048 | GGGTGGTGGCTATGGCTTT |
| 6049 | AAGCCATAGCCACCACCCT | 6050 | AGGGTGGTGGCTATGGCTT |
| 6051 | AGCCATAGCCACCACCCTC | 6052 | GAGGGTGGTGGCTATGGCT |
| 6053 | GCCATAGCCACCACCCTCA | 6054 | TGAGGGTGGTGGCTATGGC |
| 6055 | CCATAGCCACCACCCTCAG | 6056 | CTGAGGGTGGTGGCTATGG |
| 6057 | CATAGCCACCACCCTCAGT | 6058 | ACTGAGGGTGGTGGCTATG |
| 6059 | ATAGCCACCACCCTCAGTC | 6060 | GACTGAGGGTGGTGGCTAT |
| 6061 | TAGCCACCACCCTCAGTCC | 6062 | GGACTGAGGGTGGTGGCTA |
| 6063 | AGCCACCACCCTCAGTCCC | 6064 | GGGACTGAGGGTGGTGGCT |
| 6065 | GCCACCACCCTCAGTCCCT | 6066 | AGGGACTGAGGGTGGTGGC |
| 6067 | CCACCACCCTCAGTCCCTG | 6068 | CAGGGACTGAGGGTGGTGG |
| 6069 | CACCACCCTCAGTCCCTGG | 6070 | CCAGGGACTGAGGGTGGTG |
| 6071 | ACCACCCTCAGTCCCTGGA | 6072 | TCCAGGGACTGAGGGTGGT |
| 6073 | CCACCCTCAGTCCCTGGAG | 6074 | CTCCAGGGACTGAGGGTGG |
| 6075 | CACCCTCAGTCCCTGGAGC | 6076 | GCTCCAGGGACTGAGGGTG |
| 6077 | ACCCTCAGTCCCTGGAGCT | 6078 | AGCTCCAGGGACTGAGGGT |
| 6079 | CCCTCAGTCCCTGGAGCTT | 6080 | AAGCTCCAGGGACTGAGGG |
| 6081 | CCTCAGTCCCTGGAGCTTC | 6082 | GAAGCTCCAGGGACTGAGG |
| 6083 | CTCAGTCCCTGGAGCTTCC | 6084 | GGAAGCTCCAGGGACTGAG |
| 6085 | TCAGTCCCTGGAGCTTCCT | 6086 | AGGAAGCTCCAGGGACTGA |
| 6087 | CAGTCCCTGGAGCTTCCTC | 6088 | GAGGAAGCTCCAGGGACTG |
| 6089 | AGTCCCTGGAGCTTCCTCA | 6090 | TGAGGAAGCTCCAGGGACT |
| 6091 | GTCCCTGGAGCTTCCTCAT | 6092 | ATGAGGAAGCTCCAGGGAC |
| 6093 | TCCCTGGAGCTTCCTCATC | 6094 | GATGAGGAAGCTCCAGGGA |
| 6095 | CCCTGGAGCTTCCTCATCA | 6096 | TGATGAGGAAGCTCCAGGG |
| 6097 | CCTGGAGCTTCCTCATCAT | 6098 | ATGATGAGGAAGCTCCAGG |
| 6099 | CTGGAGCTTCCTCATCATC | 6100 | GATGATGAGGAAGCTCCAG |
| 6101 | TGGAGCTTCCTCATCATCC | 6102 | GGATGATGAGGAAGCTCCA |
| 6103 | GGAGCTTCCTCATCATCCT | 6104 | AGGATGATGAGGAAGCTCC |
| 6105 | GAGCTTCCTCATCATCCTC | 6106 | GAGGATGATGAGGAAGCTC |
| 6107 | AGCTTCCTCATCATCCTCT | 6108 | AGAGGATGATGAGGAAGCT |
| 6109 | GCTTCCTCATCATCCTCTG | 6110 | CAGAGGATGATGAGGAAGC |
| 6111 | CTTCCTCATCATCCTCTGC | 6112 | GCAGAGGATGATGAGGAAG |
| 6113 | TTCCTCATCATCCTCTGCT | 6114 | AGCAGAGGATGATGAGGAA |
| 6115 | TCCTCATCATCCTCTGCTT | 6116 | AAGCAGAGGATGATGAGGA |
| 6117 | CCTCATCATCCTCTGCTTC | 6118 | GAAGCAGAGGATGATGAGG |
| 6119 | CTCATCATCCTCTGCTTCA | 6120 | TGAAGCAGAGGATGATGAG |
| 6121 | TCATCATCCTCTGCTTCAT | 6122 | ATGAAGCAGAGGATGATGA |
| 6123 | CATCATCCTCTGCTTCATC | 6124 | GATGAAGCAGAGGATGATG |
| 6125 | ATCATCCTCTGCTTCATCC | 6126 | GGATGAAGCAGAGGATGAT |
| 6127 | TCATCCTCTGCTTCATCCT | 6128 | AGGATGAAGCAGAGGATGA |
| 6129 | CATCCTCTGCTTCATCCTC | 6130 | GAGGATGAAGCAGAGGATG |
| 6131 | ATCCTCTGCTTCATCCTCC | 6132 | GGAGGATGAAGCAGAGGAT |
| 6133 | TCCTCTGCTTCATCCTCCC | 6134 | GGGAGGATGAAGCAGAGGA |
| 6135 | CCTCTGCTTCATCCTCCCT | 6136 | AGGGAGGATGAAGCAGAGG |
| 6137 | CTCTGCTTCATCCTCCCTG | 6138 | CAGGGAGGATGAAGCAGAG |
| 6139 | TCTGCTTCATCCTCCCTGG | 6140 | CCAGGGAGGATGAAGCAGA |
| 6141 | CTGCTTCATCCTCCCTGGC | 6142 | GCCAGGGAGGATGAAGCAG |
| 6143 | TGCTTCATCCTCCCTGGCA | 6144 | TGCCAGGGAGGATGAAGCA |
| 6145 | GCTTCATCCTCCCTGGCAT | 6146 | ATGCCAGGGAGGATGAAGC |
| 6147 | CTTCATCCTCCCTGGCATC | 6148 | GATGCCAGGGAGGATGAAG |
| 6149 | TTCATCCTCCCTGGCATCT | 6150 | AGATGCCAGGGAGGATGAA |
| 6151 | TCATCCTCCCTGGCATCTG | 6152 | CAGATGCCAGGGAGGATGA |

Results

To determine the genetic architecture of AA taking an unbiased approach in a large cohort of patients, we initiated a GWAS by selecting a discovery cohort of 256 patients with severe phenotype (AU) (FIG. 1) who reported a family history of AA and low age of onset. Cases were ascertained through the National Alopecia Areata Registry (NAAR)^{N9} and allele frequencies were compared to previously genotyped controls.^{N10} Genome-wide association tests adjusted for residual population stratification (λ=1.036) identified 53 SNPs significantly associated with AA (p≦5×10⁻⁷), half of which were located within the HLA. For our replication study, we next genotyped a cohort of 832 NAAR patients, containing all subsets of disease severity. Controls were obtained from CGEMS (http://cgems.cancer.gov/data/).^{N11,N12} Genome-wide association tests adjusted for residual population stratification (λ=1.032) identified 93 SNPs which were significantly associated with AA (p≦5×10⁻⁷). Finally, we performed a joint analysis of the 1055 AA cases and 3278 controls, and genome-wide association tests adjusted for residual population stratification (λ=1.051) identified 141 SNPs that exceeded genome-wide significance (p≦5×10⁻⁷) (FIG. 1, Table 2).

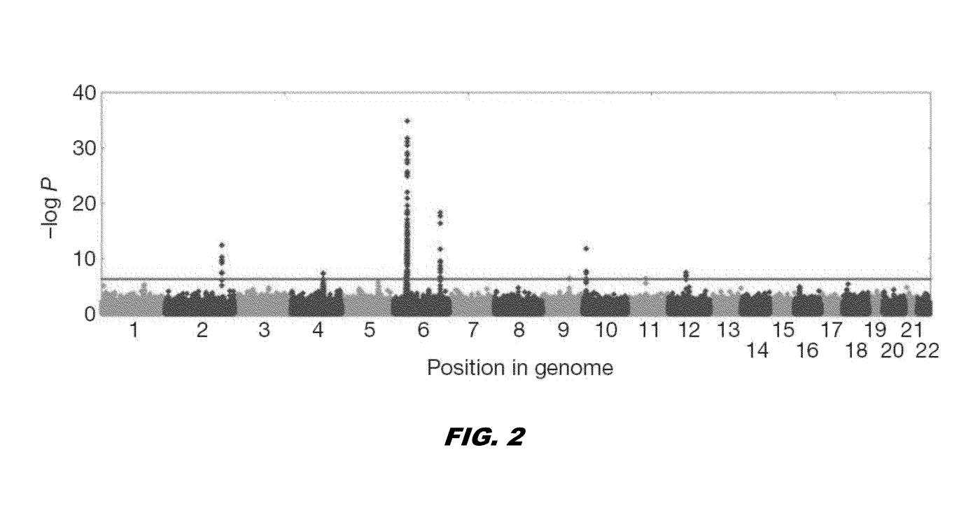

Our analysis uncovered at least ten susceptibility loci for AA, the majority of which clustered into six genomic regions and fell within discrete haplotype blocks (FIG. 2, Table 3). These include loci on chromosome 2q33.2 containing the CTLA4 gene; 4q27 containing the IL-2/IL-21 locus; 6p21.32 containing the HLA region; 6q25.1 which harbors the ULBP genes; 10p15.1 containing IL-2RA; and 12q13 containing Eos (IKZF4). Two of the remaining individual SNPs fell into discrete regions; one is located on chromosome 9q31.1 within an intron of syntaxin 17 (STX17), and the second is located on chromosome 11q13, upstream from peroxiredoxin 5 (PRDX5). Two individual SNPs in our study are also located within gene deserts. One SNP (rs361147) falls within a 560 Kb region on chromosome 4q31.3, and is bounded by the PET112L and FBXW7 genes. The second SNP (rs10053502) lies within a 1.2 Mb region on chromosome 5p13.1, and is flanked by the DAB2 and PTGER4 genes. To assess additional regions that may exceed significance in future replication studies, we identified an additional 163 SNPs that were nominally significant (1×10⁻⁴<p≦5×10⁻⁷). Interestingly, these loci implicate 12 additional genes involved in the immune response, inflammation, and/or have been implicated in other autoimmune diseases, notably, IL13, IL6, IL26, IFNG, SOCS1 and PTPN2 (Table 2, Table 4). Finally, imputation analysis identified additional statistically significant SNPs within each of the 10 regions that exceeded significance (listed above) and one additional SNP in PTPN2 that raised it above statistical significance (p=3.38×10⁻⁷) (Table 4).

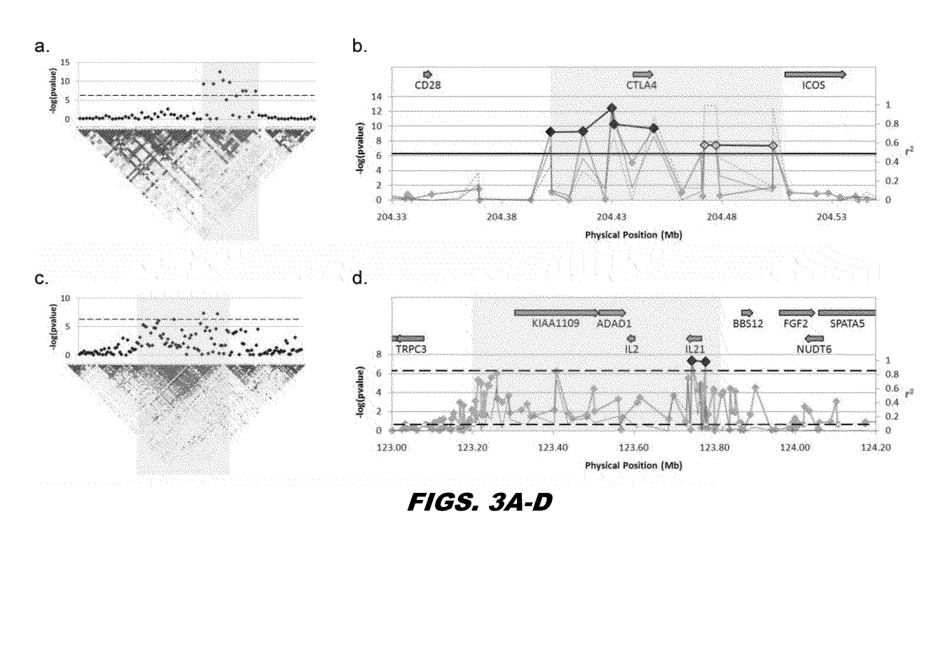

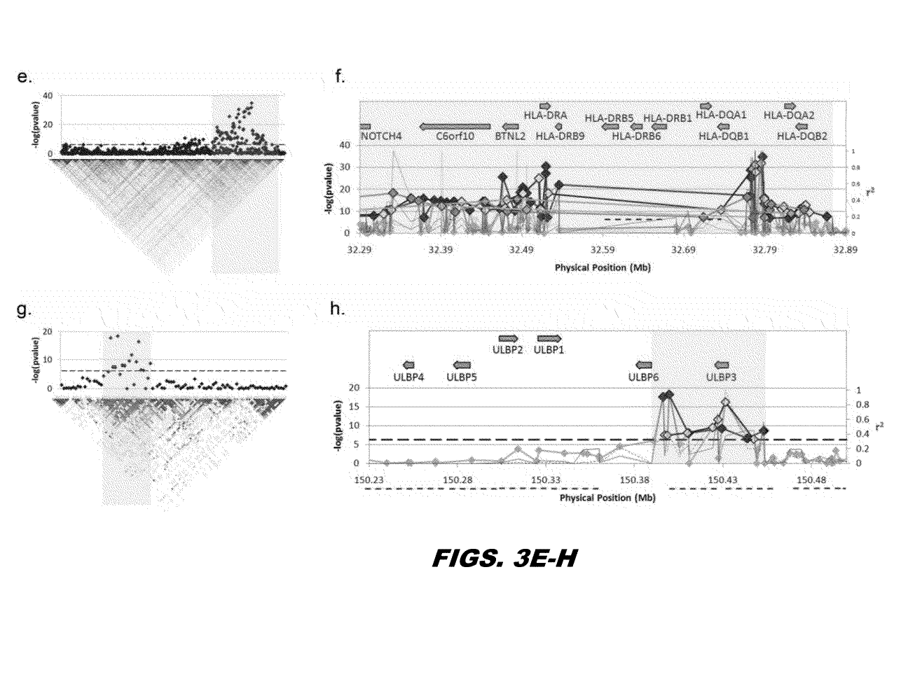

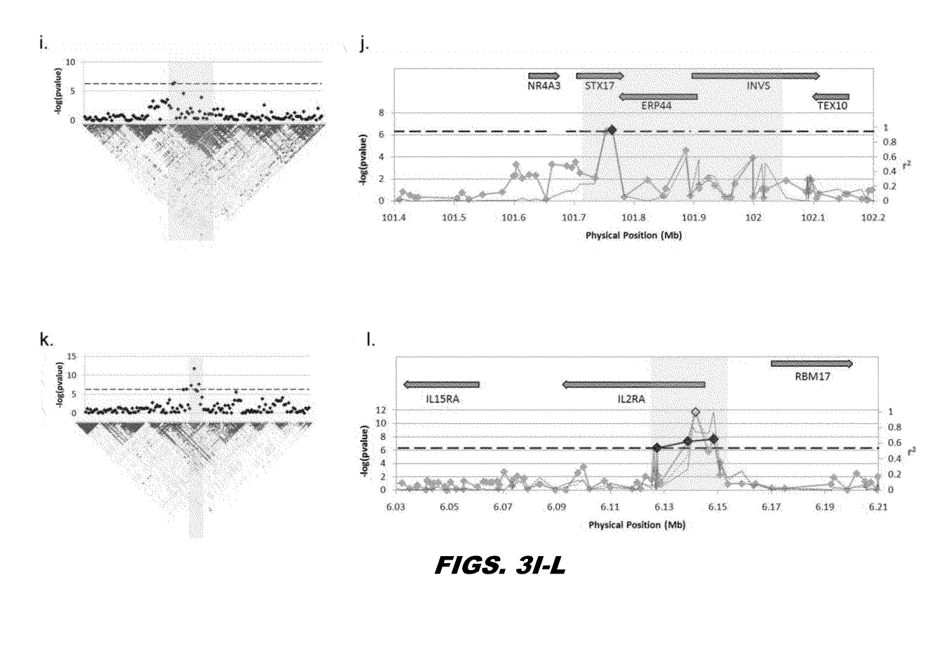

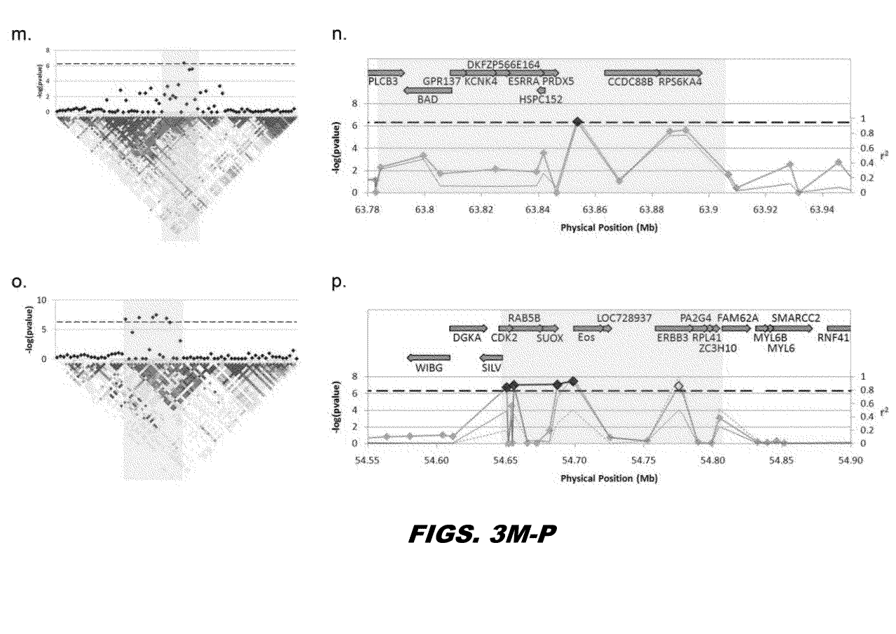

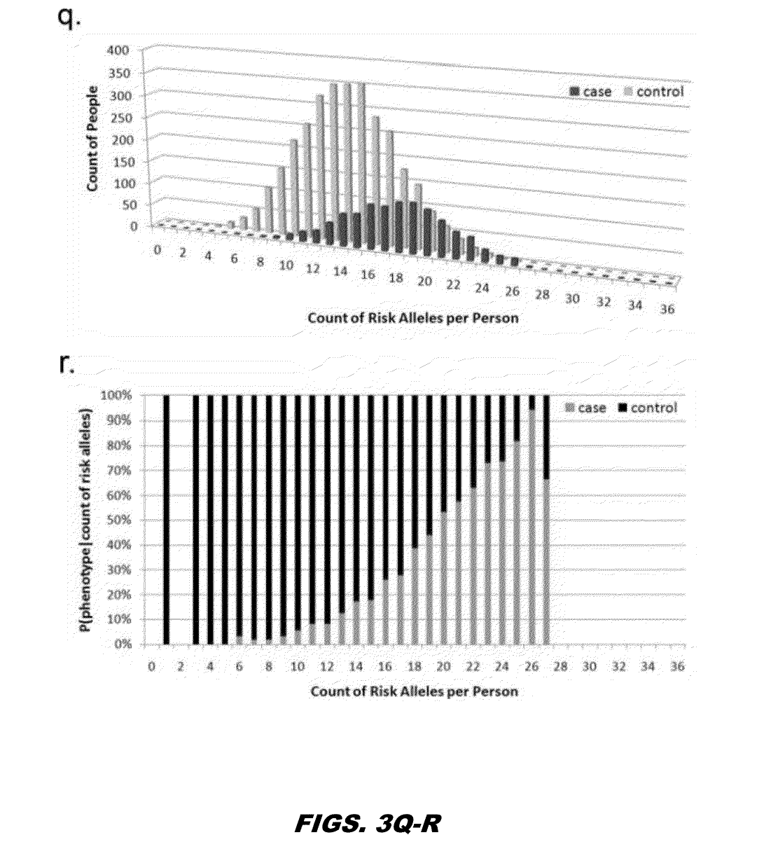

We next sought to determine the distribution of risk alleles in AA and assess the extent to which they contribute to disease. First, we reduced redundancy in our association evidence by utilizing conditional analysis to determine which SNPs represent independent risk factors within the regions we identified (FIG. 5). This analysis reduced the 141 significantly associated SNPs to a set of 18 risk haplotypes (FIG. 3 and Table 3). For each haplotype, we chose a single marker as a proxy for the haplotype. In order to assess the distribution of risk haplotypes among our cohort of AA patients and controls, we devised a new test statistic, designated as the Genetic Liability Index (GLI). Strikingly, the distribution of risk alleles is significantly different between cases and controls, wherein AA patients carry an average of 18 risk alleles, versus 14 in control individuals (FIG. 3). It is notable that the median odds ratio (OR) for our minimally redundant set of SNPs is 1.5 (ranging from 1.32-8.84), indicating stronger effects than are identified by GWAS (median OR 1.33).^{N13} To determine the relative contribution of different alleles to the genetic burden of AA, population attributable risks were calculated for genotypes of individual SNPs and show very large contributions to risk from individual alleles (ranging from 16%-91%) (Table 5). Together with the high risk in siblings,^{N5,N6} these findings document unequivocally an overwhelming contribution of risk from genetic factors in AA, and await confirmation in a validation study.

Our GWAS study in AA is the first to implicate the ULBP genes in any autoimmune disease. These ligands were originally named RAET genes (retinoic acid early transcript loci) in the mouse and ULBP (cytomegalovirus UL16-binding protein) in the human. ULBP1-6 reside in a 180 kb MHC Class I related cluster of six genes on human chromosome 6q24 that is believed to have arisen by several duplication events from the MHC locus on chromosome 6p.^{N14} Our GWAS results point to the specific haplotype block containing ULBP3 and ULBP6 as being strongly implicated in AA (FIG. 3). Importantly, each of the ULBP genes has been shown to function as a bona fide NKG2D activating ligand.^{N15,N16} NKG2D ligands, including the MICA/B genes and ULBPs, are stress-induced molecules that act as ‘danger signals’ to alert NK, NKT, δγ T, Tregs and CD8+ T lymphocytes through the engagement of the receptor NKG2D.^{N15}

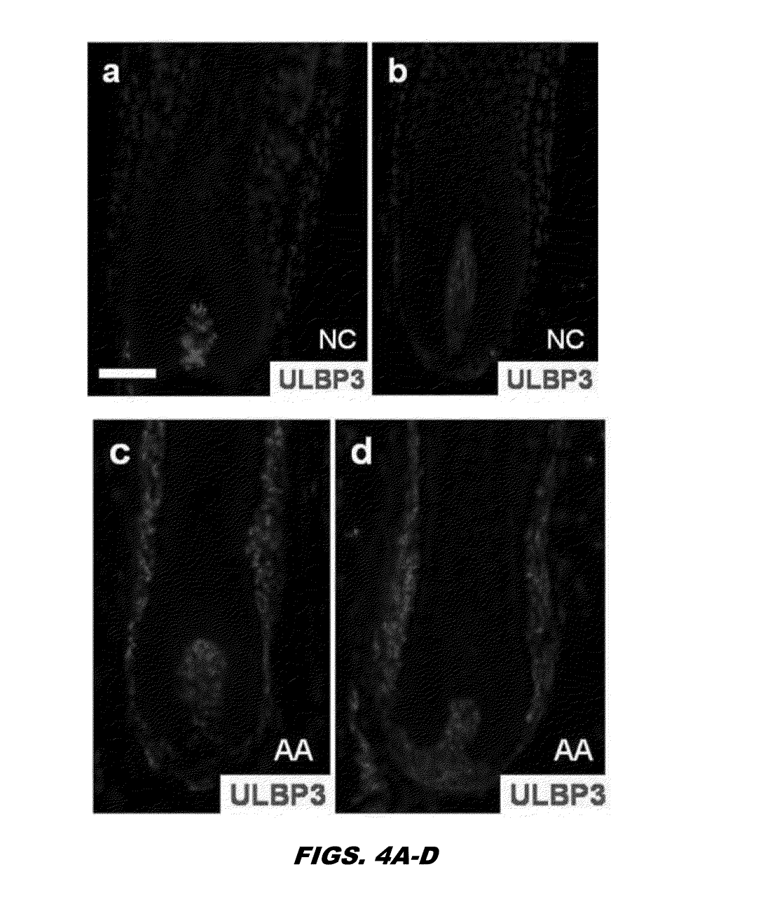

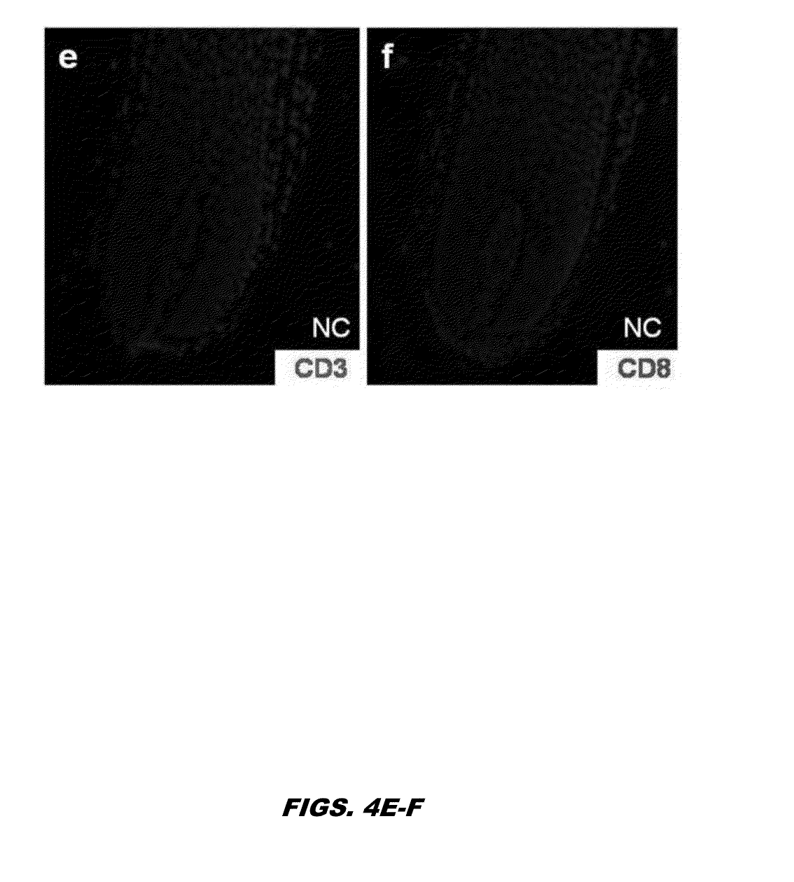

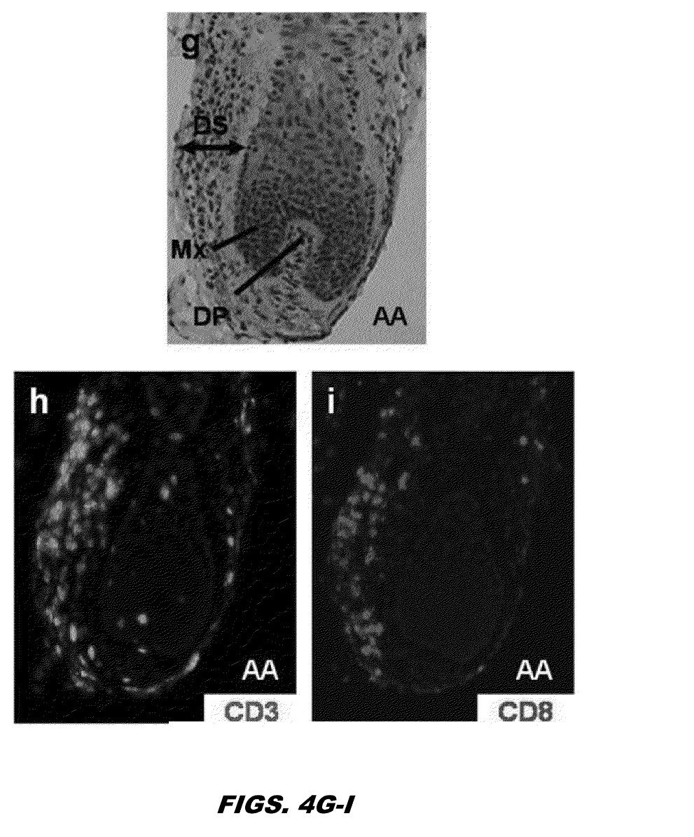

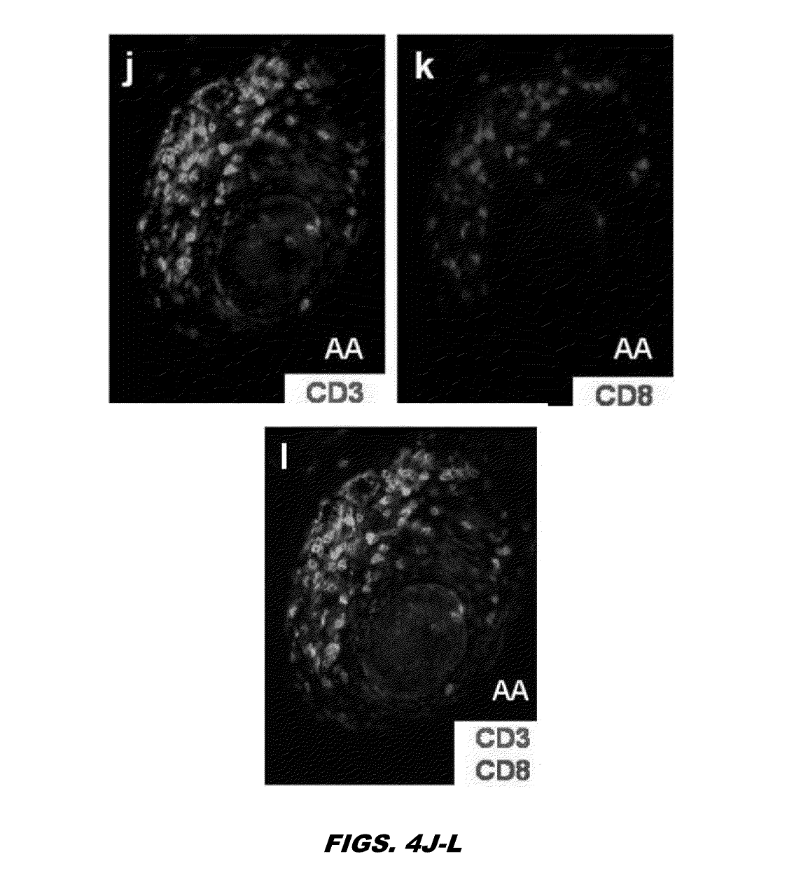

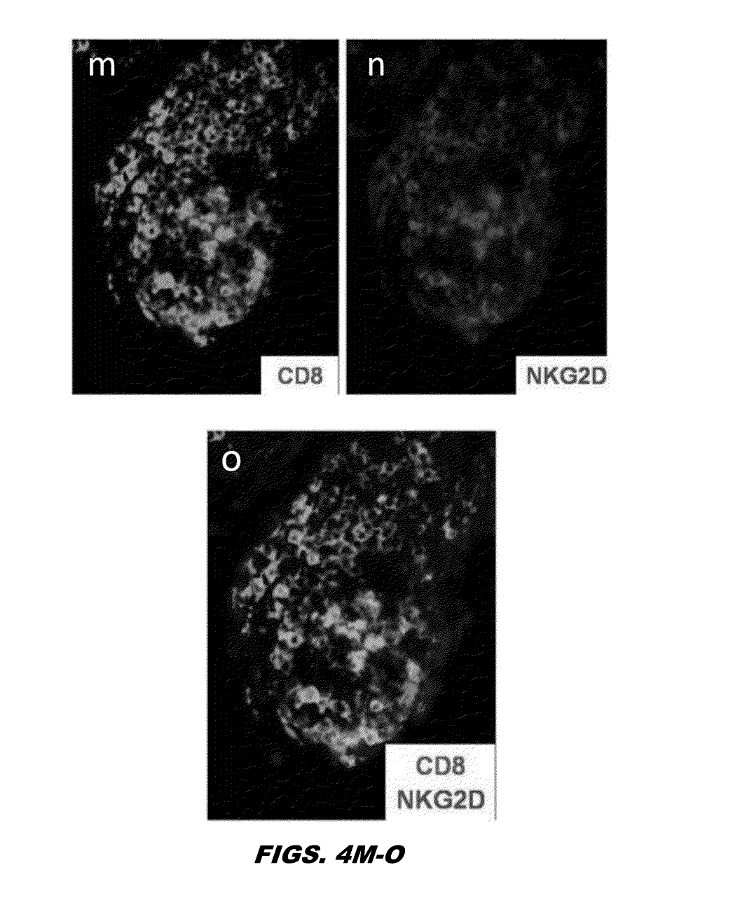

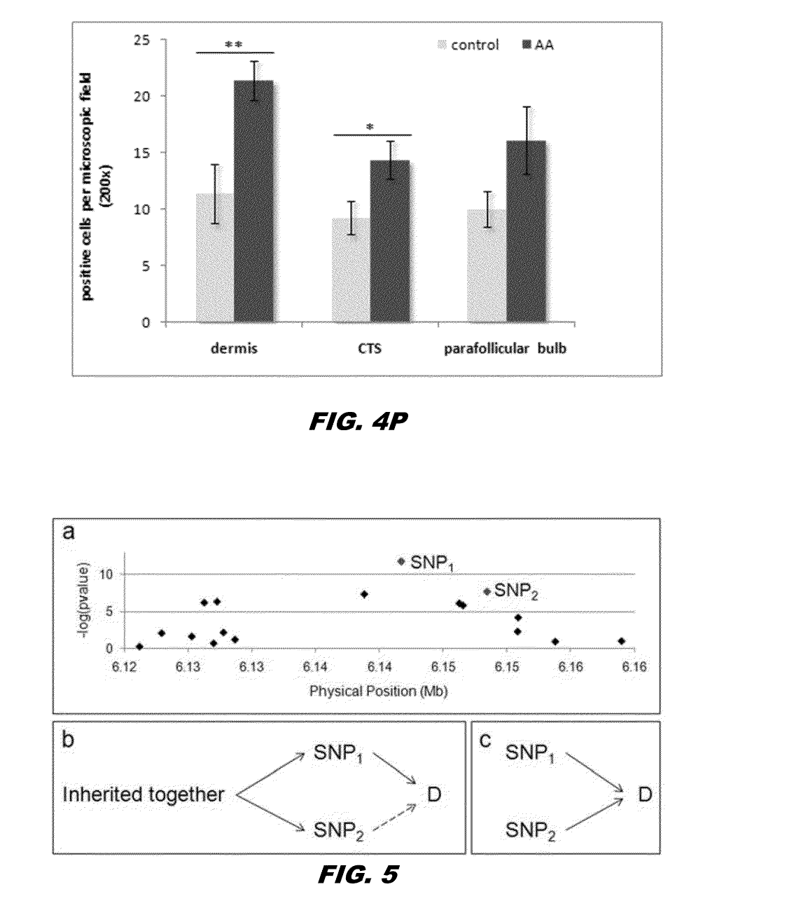

Perturbations in the hair follicle microenvironment itself contributes to the initiation of AA. NKG2D ligands, therefore, if overexpressed in genetically susceptible individuals, can trigger an autoimmune response against the tissue or organ expressing the ligand.^{N17} To probe this in the setting of AA, we examined the distribution of ULBP3 protein within the hair follicle of unaffected scalp (FIGS. 4A-B) and in the hair follicles of AA patients (FIGS. 4C-D). Whereas ULBP is expressed at low levels with the hair follicle dermal papilla in normal hair follicles (FIGS. 4A-B), strikingly, in two different patients with early active AA lesions, we observed marked upregulation of ULBP3 expression in the dermal sheath as well as the dermal papilla (FIGS. 4B-C). We then replicated this finding in a cohort of 16 independent AA patients from various stages of disease compared with scalp biopsies of 7 control individuals. Quantitative immunohistomorphometry corroborated a significantly increased number of ULBP3 positive cells in the dermal sheath and dermis in AA skin samples compared to controls (FIG. 4P). A massive inflammatory cell infiltrate was noted within the dermal sheath characterized by CD8+CD3+ T cells (FIGS. 4G-L), but only rare NK cells. Finally, double immunostaining with an anti-CD8 and an anti-NKG2D antibodies revealed that most CD8+ T cells co-expressed NKG2D (FIGS. 4M-O). The autoimmune attack in AA region is mediated by CD8+NKG2D+ cytotoxic T cells of which infiltration may be induced by upregulation of the NKG2D ligand ULBP3 in the dermal sheath of the HF. Ectopic and excessive expression of ULBP3 in the dermal sheath of the hair follicle in active lesions may be one of the most significant abnormalities of the HF signaling landscape in AA.

The localization of an NK activating ligand in the outermost layer of the hair follicle places it in an ideal position to express a ‘danger signal’ and engage NKG2D on immune cells in the local milieu. Transient inducible overexpression of another NKG2D ligand, Rae-1, in the epidermis of mice was previously shown to dramatically alter the immune landscape within the skin^{N18}, suggesting that the acute upregulation of ULBP3 in response to stress or danger may have a similar effect on initiating hair follicle autoimmunity in AA. Consistent with these findings, Ito and colleagues demonstrated a massive upregulation of the NK ligand MIC/A in the hair follicles of patients with AA.^{N4} Taken together with the increased numbers of perifollicular NKG2D+ CD8+ cells that we and others observed in lesional skin of AA patients (FIG. 4),^{N19,N20} these data implicate a new mechanism involving recruitment of NKG2D-expressing cells in the etiology of AA, which may contribute to the collapse of immune privilege of the hair follicle.

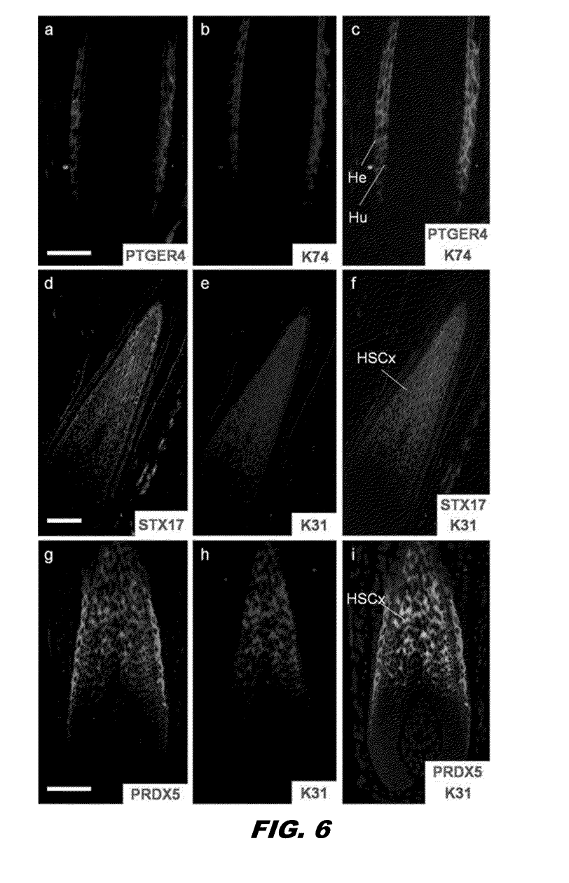

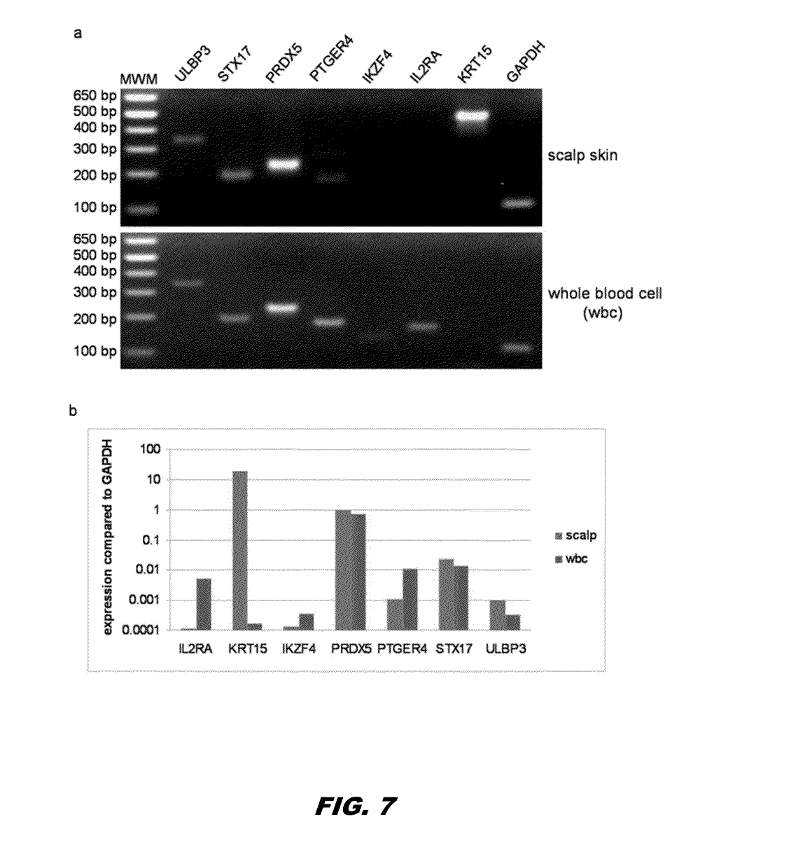

In addition to ULBP3/ULBP6, we identified several other genes that are expressed in the hair follicle and may provide insight into the initiating events (FIG. 6 and FIG. 7). For example, syntaxin 17 (STX17) (p=3.60×10⁻⁷) is widely though weakly expressed in the hair follicle.^{N21} This gene is associated with the grey hair phenotype in horses, which is of interest because AA is known to attack pigmented hair follicles.^{N22} Peroxiredoxin 5 (PRDX5) (p=4.14×10⁻⁷), is an antioxidant enzyme involved in the cellular response to oxidative stress that has been implicated in the degeneration of the target cells (astrocytes) of another autoimmune disorder, MS.^{N23} The prostaglandin E4 (EP4) receptor (PTGER4) is highly expressed in the hair follicle outer root sheath, inner root sheath and cortex, as well as the interfollicular epidermis.^{N24} Another SNP in our GWAS resides in a gene desert identified in Crohn's disease^{N25,N26} and multiple sclerosis^{N27} and shown to contain a regulator of PTGER4 gene expression. Prostaglandin E2-EP4 signaling plays a key role in the initiation of skin immune responses by promoting the migration of Langerhans cells, increasing their expression of costimulatory molecules and amplifying their ability to stimulate T cells.^{N28} Taken together, we found evidence for several genes whose robust expression in the hair follicle could contribute to a disruption in the local milieu, resulting in the collapse of immune privilege and the onset of autoimmunity.

Discussion

The results of the GWAS implicate both innate and adaptive immunity in the pathogenesis of disease in AA (Table 1). In Table 1, each of the 10 regions that display significant association to AA were summarized. For each gene implicated by this study, diseases are listed for which a GWAS or previous candidate gene study identified the same region. Information is obtained from the Human Genetic Epidemiology Navigator (www.huge navigator.net) and the OPG catalogue of GWAS (www.genome.gov).

The data further implicate several factors that conspire to induce and promote immune dysregulation in the pathogenesis of AA. Strong evidence was found for genes involved in the differentiation and maintenance of both immunosuppressive Tregs, as well as their functional antagonists, pro-inflammatory T helper cells (Th17). Tregs play a critical role in preventing immune responses against autoantigens, and their differentiation depends on the early expression of IL2RA/CD25 (p=1.74×10⁻¹²), as well as a key lineage-determining transcription factor, Foxp3. Foxp3-mediated gene silencing is critical in determining that Tregs effectively suppress immune responses.^{N29} Both IL-2 (p=4.27×10⁻⁰⁸) and its high affinity receptor IL-2RA (p=1.74×10⁻¹²) play a central role in controlling the survival and proliferation of Tregs. Eos (IKZF4) (p=3.21×10⁻⁸), a member of the Ikaros family of transcription factors, is a key co-regulator of FoxP3 directed gene silencing during Treg differentiation. While Tregs utilize several different mechanisms to suppress immune responses, the high expression of CTLA4 (p=3.55×10⁻¹³), may be a major determinant of their suppressive activity, particularly since CTLA4 is essential for the inhibitory activity of Tregs on antigen presenting cells.^{N30} The IL-2 locus is tightly linked with IL-21 (p=4.27×10⁻⁰⁸), which has pleiotropic effects on multiple cell lineages, including CD8+ T cells, B cells, NK cells, and dendritic cells. IL-21 is a major product of proinflammatory Th17 (IL-17-producing CD4(+) T helper cells) and has been shown to play a key role in both promoting the differentiation of Th17 cells as well as limiting the differentiation of Tregs.^{N31} Collectively, the constellation of immunoregulatory genes implicated in AA shift the focus squarely on the importance of Tregs and Th17 cells as targets for future studies and therapeutic targeting.

The ‘common cause hypothesis’ of autoimmune diseases has received tremendous validation from GWAS in recent years.^{N32} This hypothesis evolved initially from epidemiological studies that demonstrated the aggregation of different autoimmune diseases within families and was further supported by the finding of common susceptibility regions in linkage studies. Our G WAS upheld the previously reported robust associations of HLA genes in AA and other autoimmune disorders, in particular, HLA-DRA (p=2.93×10⁻³¹ and HLA-DQA2 (p=1.38×10⁻³⁵), as well as a report of MICA and NOTCH4, and outside the HLA, PTPN22 (p=1.98×10⁴) (reviewed in^{N3}), whereas we did not find evidence for any of the other loci previously tested in AA using the candidate gene approach (Table 6). Prior to this GWAS, we performed linkage analysis in a cohort of 28 AA families.^{N8} Our GWAS evidence coincides with linkage at the loci on 6p (HLA), 6q (ULBPs), 10p (IL2RA), and 18p (PTPN2). In accordance with the common cause hypothesis, our GWAS revealed a number of risk loci in common with other forms of autoimmunity, such as rheumatoid arthritis (RA), type I diabetes (T1D), celiac disease (CeD), systemic lupus erythematosus (SLE), multiple sclerosis (MS) and psoriasis (PS), in particular, CTLA4, IL2/IL2RA, IL21 and genes critical to Treg maintenance (Table 1, Table 3, Table 4). The commonality with RA, T1D, and CeD in particular, is especially noteworthy in light of the significance of the NKG2D receptor in the pathogenesis of each of these three diseases.^{N17}

Our GWAS establishes the genetic basis of AA for the first time, revealing at least 10 loci that contribute to disease. These findings open new avenues of exploration for therapy based on the underlying mechanisms of AA with a focus not only on T cell subsets and mechanisms common to other forms of autoimmunity, but also on unique mechanisms that involve signaling pathways downstream of the NKG2D receptor.

TABLE 1
Genes with significant association to AA.

| | | | Stongest | Maximum | |
|---|---|---|---|---|---|
| | | | association | odds | |
| Region | Gene | Function | (pvalue) | ratio | Involved in other autoimmune disease |
| 2q33.2 | CTLA4 | T-cell proliferation | 3.55 × 10⁻¹³ | 1.44 | T1D, RA, CeD, MS, SLE, GD |
| | ICOS | T-cell proliferation | 4.33 × 10⁻⁰⁸ | 1.32 | |
| 4q27 | IL21/IL2 | T-, B- and NK-cell | 4.27 × 10⁻⁰⁸ | 1.34 | T1D, RA, CeD, PS |
| | | proliferation | | | |
| 6q25.1 | ULBP6 | NKG2D activating ligand | 4.49 × 10⁻¹⁹ | 1.65 | none |
| | ULBP3 | NKG2D activating ligand | 4.43 × 10⁻¹⁷ | 1.52 | none |
| 9q31.1 | STX17 | premature hair graying | 3.60 × 10⁻⁰⁷ | 1.33 | none |
| 10p15.1 | IL2RA | T-cell proliferation | 1.74 × 10⁻¹² | 1.41 | T1D, MS, GD |
| 11q13 | PRDX5 | antioxidant enzyme | 4.14 × 10⁻⁰⁷ | 1.33 | MS |
| 12q13 | Eos | T-cell proliferation | 3.21 × 10⁻⁰⁸ | 1.34 | T1D, SLE |
| | (IKZF4) | | | | |
| 6p21.32 | MICA | NK cell activation | 1.19 × 10⁻⁰⁷ | 1.44 | T1D, RA, CeD, UC, PS, SLE |
| (HLA) | NOTCH4 | T-cell differentiation | 1.03 × 10⁻⁰⁸ | 1.61 | T1D, RA, MS |
| | C6orf10 | | 1.45 × 10⁻¹⁶ | 2.36 | T1D, RA, PS |
| | BTNL2 | T-cell proliferation | 2.11 × 10⁻²⁶ | 2.7 | T1D, RA, UC, CD, SLE, MS |
| | HLA-DRA | Antigen presentation | 2.93 × 10⁻³¹ | 2.62 | T1D, RA, CeD, MS |
| | HLA-DQA1 | Antigen presentation | 3.60 × 10⁻¹⁷ | 2.15 | T1D, RA, CeD, MS, SLE, PS, CD, UC, GD |
| | HLA-DQA2 | Antigen presentation | 1.38 × 10⁻³⁵ | 5.43 | T1D, RA |
| | HLA-DQB2 | Antigen presentation | 1.73 × 10⁻¹³ | 1.6 | RA |
| | HLA-DOB | Antigen presentation | 2.07 × 10⁻⁰⁸ | 1.72 | |

The p-value of the most significant SNP, and the OR for the SNP with the largest effect estimate are listed. Diseases are listed for which a GWAS or previous candidate gene study identified the same region: type I diabetes (T1D), rheumatoid arthritis (RA), celiac disease (CeD), multiple sclerosis (MS), system lupus erythematosus (SLE), Graves disease (GD), psoriasis (PS), Crohn's disease (CD), and ulcerative colitis (UC).

TABLE 2
Association results for SNPs that exceed significance level of p = 1 × 10⁻⁴.

| | | | | | Refer- | | | | 95% | 95% |
|---|---|---|---|---|---|---|---|---|---|---|
| | | position | Gene | | ence | MAF | MAF | Odds | CI | CI |
| Chr | SNP | (bp) | Symbol | pvalue | allele | controls | cases | Ratio | lower | upper |
| 1 | rs2275909 | 6,991,259 | CAMTA1 | 8.77E−06 | G | 0.31 | 0.36 | 1.28 | 1.15 | 1.42 |
| 1 | rs12060498 | 166,053,889 | SAC | 8.31E−05 | A | 0.17 | 0.13 | 0.74 | 0.64 | 0.86 |
| 1 | rs16828608 | 176,396,522 | RASAL2 | 1.04E−05 | A | 0.09 | 0.13 | 1.43 | 1.23 | 1.67 |
| 1 | rs6701848 | 176,423,439 | RASAL2 | 4.16E−05 | C | 0.09 | 0.12 | 1.40 | 1.20 | 1.64 |
| 1 | rs12036491 | 176,430,274 | RASAL2 | 8.03E−05 | A | 0.09 | 0.12 | 1.39 | 1.19 | 1.62 |
| 1 | rs11590951 | 176,589,380 | RASAL2 | 5.18E−06 | A | 0.09 | 0.12 | 1.46 | 1.25 | 1.72 |
| 1 | rs12161419 | 176,645,663 | RASAL2 | 8.72E−05 | C | 0.09 | 0.12 | 1.39 | 1.18 | 1.62 |
| 2 | rs952810 | 7,287,647 | RNF144 | 8.26E−05 | G | 0.38 | 0.43 | 1.24 | 1.12 | 1.37 |
| 2 | rs12986962 | 111,525,029 | ACOXL | 8.67E−05 | G | 0.37 | 0.32 | 0.80 | 0.72 | 0.89 |
| 2 | rs231735 | 204,402,121 | CTLA4 | 5.75E−10 | C | 0.48 | 0.40 | 0.72 | 0.65 | 0.80 |
| 2 | rs231804 | 204,416,891 | CTLA4 | 4.97E−10 | G | 0.42 | 0.35 | 0.71 | 0.64 | 0.79 |
| 2 | rs1024161 | 204,429,997 | CTLA4 | 3.55E−13 | A | 0.40 | 0.49 | 1.47 | 1.33 | 1.62 |
| 2 | rs926169 | 204,430,997 | CTLA4 | 5.50E−11 | A | 0.39 | 0.47 | 1.41 | 1.28 | 1.56 |
| 2 | rs733618 | 204,439,189 | CTLA4 | 8.26E−06 | G | 0.08 | 0.11 | 1.46 | 1.24 | 1.72 |
| 2 | rs231726 | 204,449,111 | CTLA4 | 1.94E−10 | A | 0.32 | 0.39 | 1.41 | 1.27 | 1.57 |
| 2 | rs10497873 | 204,470,572 | CTLA4 | 7.65E−07 | A | 0.22 | 0.17 | 0.72 | 0.63 | 0.82 |
| 2 | rs3096851 | 204,472,127 | CTLA4 | 3.58E−08 | C | 0.31 | 0.37 | 1.35 | 1.22 | 1.50 |
| 2 | rs3116504 | 204,477,299 | CTLA4 | 3.73E−08 | G | 0.31 | 0.37 | 1.35 | 1.22 | 1.50 |
| 2 | rs3096866 | 204,503,197 | ICOS | 4.33E−08 | G | 0.31 | 0.38 | 1.35 | 1.22 | 1.50 |
| 2 | rs10490186 | 230,189,779 | DNER | 7.51E−05 | G | 0.35 | 0.40 | 1.23 | 1.12 | 1.37 |
| 2 | rs1531968 | 240,025,939 | HDAC4 | 8.10E−05 | G | 0.37 | 0.31 | 0.81 | 0.73 | 0.89 |
| 3 | rs13088671 | 32,334,657 | CKLFSF8 | 9.27E−05 | A | 0.11 | 0.14 | 1.35 | 1.17 | 1.56 |
| 3 | rs4299518 | 45,784,277 | SLC6A20 | 1.03E−04 | G | 0.47 | 0.42 | 0.82 | 0.74 | 0.90 |
| 3 | rs3912834 | 55,017,401 | CACNA2D3 | 7.28E−05 | G | 0.15 | 0.12 | 0.74 | 0.63 | 0.85 |
| 3 | rs7638884 | 56,987,278 | SPATA12 | 3.87E−05 | A | 0.42 | 0.47 | 1.25 | 1.13 | 1.38 |
| 3 | rs9818327 | 118,337,491 | LSAMP | 2.03E−05 | A | 0.28 | 0.23 | 0.77 | 0.68 | 0.87 |
| 3 | rs7649284 | 118,364,404 | LSAMP | 4.74E−05 | G | 0.27 | 0.23 | 0.78 | 0.70 | 0.88 |
| 4 | rs6839274 | 3,130,428 | HD | 8.71E−05 | G | 0.11 | 0.08 | 0.70 | 0.59 | 0.84 |
| 4 | rs363097 | 3,147,057 | HD | 9.11E−05 | G | 0.12 | 0.09 | 0.71 | 0.60 | 0.84 |
| 4 | rs6822371 | 103,452,230 | SLC39A8 | 7.98E−05 | A | 0.37 | 0.32 | 0.81 | 0.73 | 0.90 |
| 4 | rs1526926 | 123,213,668 | TRPC3 | 4.46E−06 | C | 0.43 | 0.49 | 1.27 | 1.15 | 1.41 |
| 4 | rs3108402 | 123,218,902 | TRPC3 | 7.62E−06 | A | 0.26 | 0.21 | 0.75 | 0.67 | 0.85 |
| 4 | rs941130 | 123,221,219 | TRPC3 | 1.05E−05 | A | 0.26 | 0.22 | 0.76 | 0.67 | 0.86 |
| 4 | rs3108397 | 123,237,584 | TRPC3 | 1.61E−05 | A | 0.42 | 0.47 | 1.25 | 1.13 | 1.38 |
| 4 | rs3108396 | 123,241,010 | TRPC3 | 1.78E−05 | C | 0.36 | 0.32 | 0.79 | 0.71 | 0.88 |
| 4 | rs6534338 | 123,246,319 | TRPC3 | 2.33E−06 | A | 0.31 | 0.25 | 0.76 | 0.68 | 0.85 |
| 4 | rs7684834 | 123,260,318 | TRPC3 | 8.43E−07 | G | 0.38 | 0.45 | 1.30 | 1.17 | 1.43 |
| 4 | rs7683061 | 123,407,319 | Tenr | 5.42E−07 | A | 0.37 | 0.44 | 1.30 | 1.18 | 1.44 |
| 4 | rs1127348 | 123,500,310 | Tenr | 3.73E−05 | G | 0.22 | 0.18 | 0.76 | 0.67 | 0.86 |
| 4 | rs4492018 | 123,733,978 | IL21 | 2.72E−06 | A | 0.26 | 0.32 | 1.30 | 1.17 | 1.45 |
| 4 | rs7682241 | 123,743,325 | IL21 | 4.27E−08 | A | 0.34 | 0.40 | 1.34 | 1.21 | 1.48 |
| 4 | rs2221903 | 123,758,362 | IL21 | 5.36E−05 | G | 0.31 | 0.27 | 0.79 | 0.71 | 0.88 |
| 4 | rs17005931 | 123,765,098 | IL21 | 1.26E−05 | A | 0.26 | 0.32 | 1.28 | 1.15 | 1.43 |
| 4 | rs1398553 | 123,767,518 | IL21 | 5.21E−05 | A | 0.31 | 0.27 | 0.79 | 0.71 | 0.88 |
| 4 | rs6840978 | 123,774,157 | IL21 | 2.42E−05 | A | 0.21 | 0.16 | 0.75 | 0.65 | 0.85 |
| 4 | rs2137497 | 123,777,704 | IL21 | 5.34E−08 | A | 0.39 | 0.46 | 1.33 | 1.20 | 1.47 |
| 4 | rs13110000 | 123,797,510 | IL21 | 4.09E−05 | G | 0.41 | 0.36 | 0.80 | 0.72 | 0.89 |
| 4 | rs4833253 | 123,798,300 | IL21 | 8.44E−05 | G | 0.16 | 0.20 | 1.30 | 1.15 | 1.48 |
| 4 | rs6836610 | 123,821,147 | FLJ35630 | 7.70E−05 | A | 0.30 | 0.35 | 1.24 | 1.12 | 1.38 |
| 4 | rs309406 | 123,838,157 | FLJ35630 | 3.38E−05 | G | 0.42 | 0.36 | 0.80 | 0.72 | 0.89 |
| 4 | rs368931 | 123,851,047 | FLJ35630 | 7.29E−05 | C | 0.40 | 0.34 | 0.81 | 0.73 | 0.89 |
| 4 | rs309375 | 123,900,606 | FLJ35630 | 2.74E−05 | C | 0.42 | 0.36 | 0.80 | 0.72 | 0.88 |
| 4 | rs304652 | 124,301,671 | SPATA5 | 1.68E−05 | G | 0.15 | 0.20 | 1.33 | 1.17 | 1.51 |
| 4 | rs2201997 | 124,398,692 | SPATA5 | 1.84E−05 | C | 0.15 | 0.20 | 1.33 | 1.17 | 1.51 |
| 4 | rs7670452 | 124,404,063 | SPATA5 | 3.27E−05 | G | 0.15 | 0.20 | 1.32 | 1.16 | 1.50 |
| 4 | rs11735364 | 124,405,129 | SPATA5 | 9.84E−05 | A | 0.19 | 0.23 | 1.28 | 1.13 | 1.44 |
| 4 | rs6813125 | 124,489,366 | SPATA5 | 1.57E−05 | C | 0.17 | 0.22 | 1.32 | 1.16 | 1.49 |
| 4 | rs6841700 | 124,494,160 | SPATA5 | 4.92E−05 | C | 0.20 | 0.24 | 1.28 | 1.14 | 1.44 |
| 4 | rs6552275 | 179,246,616 | AGA | 8.71E−05 | A | 0.26 | 0.31 | 1.25 | 1.12 | 1.40 |
| 4 | rs902176 | 185,891,314 | MLF1IP | 8.95E−05 | A | 0.15 | 0.18 | 1.31 | 1.15 | 1.49 |
| 4 | rs6851816 | 185,891,831 | MLF1IP | 5.36E−05 | G | 0.17 | 0.21 | 1.30 | 1.15 | 1.48 |
| 5 | rs16895538 | 61,157,916 | FLJ37543 | 7.79E−05 | A | 0.12 | 0.15 | 1.34 | 1.16 | 1.54 |
| 5 | rs11746773 | 61,162,143 | FLJ37543 | 3.46E−05 | G | 0.09 | 0.13 | 1.39 | 1.19 | 1.62 |
| 5 | rs13153954 | 61,198,236 | FLJ37543 | 9.99E−05 | G | 0.14 | 0.18 | 1.31 | 1.15 | 1.49 |
| 5 | rs6859438 | 71,049,222 | CART | 9.17E−05 | A | 0.03 | 0.05 | 1.66 | 1.29 | 2.13 |
| 5 | rs1295686 | 132,023,742 | IL13 | 7.13E−06 | A | 0.20 | 0.25 | 1.31 | 1.17 | 1.47 |
| 5 | rs20541 | 132,023,863 | IL13 | 1.87E−06 | A | 0.20 | 0.25 | 1.34 | 1.19 | 1.50 |
| 5 | rs2285700 | 132,067,031 | KIF3A | 7.78E−05 | C | 0.26 | 0.31 | 1.25 | 1.12 | 1.39 |
| 5 | rs2074529 | 132,084,046 | KIF3A | 4.05E−05 | C | 0.27 | 0.31 | 1.26 | 1.13 | 1.40 |
| 5 | rs247459 | 133,410,355 | VDAC1 | 1.10E−05 | A | 0.22 | 0.27 | 1.30 | 1.16 | 1.46 |
| 5 | rs7702415 | 133,850,977 | PHF15 | 5.16E−05 | G | 0.22 | 0.26 | 1.28 | 1.14 | 1.43 |
| 5 | rs1421630 | 163,466,086 | MAT2B | 6.26E−05 | A | 0.32 | 0.27 | 0.79 | 0.71 | 0.88 |
| 6 | rs11967812 | 29,943,610 | HLA-G | 1.07E−04 | G | 0.04 | 0.06 | 1.57 | 1.26 | 1.96 |
| 6 | rs2524005 | 30,007,656 | HLA-A | 1.00E−04 | A | 0.20 | 0.13 | 0.72 | 0.62 | 0.84 |
| 6 | rs2428521 | 30,036,628 | HCG9 | 2.78E−05 | C | 0.47 | 0.54 | 1.26 | 1.13 | 1.39 |
| 6 | rs2517689 | 30,037,232 | HCG9 | 3.15E−05 | A | 0.47 | 0.54 | 1.25 | 1.13 | 1.39 |
| 6 | rs3095340 | 30,834,918 | IER3 | 9.12E−06 | C | 0.15 | 0.09 | 0.66 | 0.56 | 0.78 |
| 6 | rs3094122 | 30,836,339 | IER3 | 1.95E−06 | C | 0.22 | 0.16 | 0.71 | 0.62 | 0.81 |
| 6 | rs6911628 | 30,847,825 | IER3 | 2.80E−07 | A | 0.27 | 0.19 | 0.71 | 0.63 | 0.81 |
| 6 | rs3131043 | 30,866,445 | IER3 | 5.10E−06 | G | 0.44 | 0.38 | 0.77 | 0.69 | 0.86 |
| 6 | rs886424 | 30,889,981 | IER3 | 9.73E−06 | A | 0.12 | 0.07 | 0.61 | 0.50 | 0.74 |
| 6 | rs2844659 | 30,932,511 | DDR1 | 1.03E−04 | A | 0.19 | 0.13 | 0.74 | 0.64 | 0.85 |
| 6 | rs2240804 | 31,028,869 | DPCR1 | 1.04E−04 | A | 0.35 | 0.41 | 1.24 | 1.11 | 1.37 |
| 6 | rs3095089 | 31,041,773 | DPCR1 | 5.03E−07 | A | 0.17 | 0.11 | 0.66 | 0.56 | 0.77 |
| 6 | rs3130544 | 31,166,319 | C6orf15 | 7.44E−05 | A | 0.11 | 0.06 | 0.64 | 0.52 | 0.78 |
| 6 | rs2233956 | 31,189,184 | C6orf15 | 2.00E−06 | G | 0.18 | 0.11 | 0.65 | 0.56 | 0.77 |
| 6 | rs7750641 | 31,237,289 | TCF19 | 7.52E−05 | A | 0.11 | 0.06 | 0.64 | 0.52 | 0.78 |
| 6 | rs2442749 | 31,460,019 | MICA | 1.19E−07 | G | 0.28 | 0.22 | 0.71 | 0.63 | 0.80 |
| 6 | rs3749946 | 31,556,841 | HCP5 | 1.68E−05 | A | 0.08 | 0.06 | 0.64 | 0.52 | 0.78 |
| 6 | rs3099844 | 31,556,955 | HCP5 | 8.60E−05 | A | 0.12 | 0.07 | 0.65 | 0.54 | 0.79 |
| 6 | rs2516399 | 31,589,278 | MICB | 6.79E−05 | G | 0.10 | 0.14 | 1.38 | 1.18 | 1.60 |
| 6 | rs2246986 | 31,590,182 | MICB | 1.89E−05 | G | 0.10 | 0.13 | 1.41 | 1.21 | 1.65 |
| 6 | rs2239705 | 31,621,381 | ATP6V1G2 | 3.32E−05 | A | 0.18 | 0.23 | 1.31 | 1.16 | 1.48 |
| 6 | rs2260000 | 31,701,455 | BAT2 | 3.82E−06 | G | 0.38 | 0.45 | 1.28 | 1.16 | 1.42 |
| 6 | rs1046089 | 31,710,946 | BAT2 | 5.92E−05 | A | 0.33 | 0.27 | 0.79 | 0.70 | 0.88 |
| 6 | rs9267522 | 31,711,749 | BAT2 | 8.88E−05 | G | 0.18 | 0.13 | 0.73 | 0.63 | 0.85 |
| 6 | rs1077393 | 31,718,508 | BAT3 | 5.89E−08 | A | 0.51 | 0.42 | 0.75 | 0.67 | 0.83 |
| 6 | rs805303 | 31,724,345 | BAT3 | 1.91E−07 | A | 0.36 | 0.29 | 0.74 | 0.66 | 0.82 |
| 6 | rs3117582 | 31,728,499 | BAT3 | 5.20E−07 | C | 0.10 | 0.05 | 0.54 | 0.43 | 0.67 |
| 6 | rs1266071 | 31,777,475 | BAT5 | 1.90E−05 | A | 0.10 | 0.14 | 1.40 | 1.20 | 1.62 |
| 6 | rs805294 | 31,796,196 | LY6G6C | 3.67E−07 | G | 0.35 | 0.28 | 0.74 | 0.66 | 0.83 |
| 6 | rs3131379 | 31,829,012 | MSH5 | 9.16E−07 | A | 0.10 | 0.05 | 0.54 | 0.43 | 0.68 |
| 6 | rs707939 | 31,834,667 | MSH5 | 9.26E−06 | A | 0.36 | 0.43 | 1.27 | 1.15 | 1.42 |
| 6 | rs707928 | 31,850,569 | C6orf27 | 1.42E−11 | G | 0.33 | 0.23 | 0.66 | 0.59 | 0.74 |
| 6 | rs2075800 | 31,885,925 | HSPA1L | 2.57E−06 | A | 0.34 | 0.42 | 1.29 | 1.17 | 1.44 |
| 6 | rs660550 | 31,945,256 | SLC44A4 | 1.16E−05 | C | 0.43 | 0.35 | 0.78 | 0.70 | 0.87 |
| 6 | rs644827 | 31,946,420 | SLC44A4 | 1.60E−05 | A | 0.43 | 0.35 | 0.78 | 0.70 | 0.87 |
| 6 | rs494620 | 31,946,692 | SLC44A4 | 3.72E−07 | A | 0.43 | 0.52 | 1.32 | 1.19 | 1.46 |
| 6 | rs2242665 | 31,947,288 | SLC44A4 | 1.36E−05 | G | 0.43 | 0.35 | 0.78 | 0.70 | 0.87 |
| 6 | rs652888 | 31,959,213 | EHMT2 | 2.58E−08 | G | 0.20 | 0.13 | 0.64 | 0.55 | 0.74 |
| 6 | rs659445 | 31,972,283 | EHMT2 | 2.78E−05 | G | 0.32 | 0.25 | 0.77 | 0.68 | 0.86 |
| 6 | rs558702 | 31,978,305 | ZBTB12 | 7.36E−07 | A | 0.10 | 0.05 | 0.54 | 0.43 | 0.67 |
| 6 | rs4151657 | 32,025,519 | BF | 3.21E−08 | G | 0.36 | 0.44 | 1.35 | 1.22 | 1.50 |
| 6 | rs1270942 | 32,026,839 | BF | 4.49E−07 | G | 0.10 | 0.05 | 0.53 | 0.43 | 0.67 |
| 6 | rs2072633 | 32,027,557 | BF | 3.60E−10 | A | 0.42 | 0.33 | 0.70 | 0.63 | 0.78 |
| 6 | rs437179 | 32,036,993 | SKIV2L | 8.48E−08 | A | 0.29 | 0.21 | 0.71 | 0.63 | 0.80 |
| 6 | rs389884 | 32,048,876 | STK19 | 4.97E−07 | G | 0.10 | 0.05 | 0.54 | 0.43 | 0.67 |
| 6 | rs6941112 | 32,054,593 | STK19 | 7.50E−11 | A | 0.33 | 0.42 | 1.43 | 1.29 | 1.59 |
| 6 | rs389883 | 32,055,439 | STK19 | 9.05E−08 | C | 0.29 | 0.21 | 0.71 | 0.63 | 0.80 |
| 6 | rs185819 | 32,158,045 | TNXB | 9.76E−07 | A | 0.49 | 0.41 | 0.77 | 0.69 | 0.85 |
| 6 | rs2269426 | 32,184,477 | TNXB | 7.08E−10 | A | 0.40 | 0.50 | 1.39 | 1.25 | 1.54 |
| 6 | rs8111 | 32,191,153 | CREBL1 | 2.01E−08 | A | 0.29 | 0.38 | 1.37 | 1.23 | 1.53 |
| 6 | rs1035798 | 32,259,200 | AGER | 5.57E−05 | A | 0.26 | 0.32 | 1.26 | 1.13 | 1.41 |
| 6 | rs2070600 | 32,259,421 | AGER | 1.15E−10 | A | 0.04 | 0.08 | 1.98 | 1.61 | 2.44 |
| 6 | rs9267833 | 32,285,878 | NOTCH4 | 3.35E−05 | G | 0.28 | 0.34 | 1.26 | 1.14 | 1.41 |
| 6 | rs2071286 | 32,287,874 | NOTCH4 | 1.47E−05 | A | 0.23 | 0.29 | 1.30 | 1.16 | 1.45 |
| 6 | rs206015 | 32,290,737 | NOTCH4 | 9.66E−05 | A | 0.11 | 0.14 | 1.35 | 1.16 | 1.56 |
| 6 | rs377763 | 32,307,122 | NOTCH4 | 1.03E−08 | A | 0.21 | 0.14 | 0.65 | 0.57 | 0.75 |
| 6 | rs3130299 | 32,311,515 | NOTCH4 | 9.19E−05 | G | 0.27 | 0.33 | 1.25 | 1.12 | 1.39 |
| 6 | rs412657 | 32,319,063 | LOC401252 | 6.99E−06 | C | 0.44 | 0.39 | 0.78 | 0.70 | 0.87 |
| 6 | rs9267947 | 32,319,196 | LOC401252 | 2.08E−09 | G | 0.45 | 0.36 | 0.72 | 0.65 | 0.80 |
| 6 | rs17576984 | 32,320,963 | LOC401252 | 1.49E−05 | A | 0.09 | 0.06 | 0.63 | 0.51 | 0.77 |
| 6 | rs405875 | 32,323,166 | LOC401252 | 3.94E−11 | G | 0.44 | 0.55 | 1.43 | 1.29 | 1.59 |
| 6 | rs3115573 | 32,326,821 | LOC401252 | 2.63E−11 | G | 0.44 | 0.54 | 1.44 | 1.29 | 1.59 |
| 6 | rs3130315 | 32,328,663 | LOC401252 | 2.71E−11 | A | 0.44 | 0.54 | 1.43 | 1.29 | 1.59 |
| 6 | rs3130320 | 32,331,236 | LOC401252 | 5.64E−19 | A | 0.36 | 0.23 | 0.57 | 0.51 | 0.64 |
| 6 | rs3130340 | 32,352,605 | LOC401252 | 1.42E−16 | G | 0.22 | 0.11 | 0.51 | 0.44 | 0.59 |
| 6 | rs3115553 | 32,353,805 | LOC401252 | 1.49E−16 | A | 0.22 | 0.11 | 0.51 | 0.44 | 0.59 |
| 6 | rs9268132 | 32,362,632 | C6orf10 | 1.58E−15 | G | 0.40 | 0.52 | 1.54 | 1.39 | 1.70 |
| 6 | rs6935269 | 32,368,328 | C6orf10 | 1.45E−16 | G | 0.22 | 0.11 | 0.51 | 0.44 | 0.59 |
| 6 | rs7775397 | 32,369,230 | C6orf10 | 5.91E−08 | C | 0.10 | 0.05 | 0.50 | 0.40 | 0.63 |
| 6 | rs6457536 | 32,381,743 | C6orf10 | 8.44E−16 | G | 0.21 | 0.11 | 0.51 | 0.44 | 0.60 |
| 6 | rs547261 | 32,390,011 | C6orf10 | 1.73E−15 | A | 0.40 | 0.52 | 1.53 | 1.38 | 1.70 |
| 6 | rs6910071 | 32,390,832 | C6orf10 | 2.95E−13 | G | 0.18 | 0.26 | 1.58 | 1.40 | 1.78 |
| 6 | rs547077 | 32,397,296 | C6orf10 | 7.25E−15 | G | 0.40 | 0.52 | 1.52 | 1.37 | 1.69 |
| 6 | rs570963 | 32,397,572 | C6orf10 | 3.03E−05 | G | 0.11 | 0.08 | 0.67 | 0.56 | 0.81 |
| 6 | rs9368713 | 32,405,315 | C6orf10 | 4.92E−15 | G | 0.40 | 0.52 | 1.53 | 1.38 | 1.69 |
| 6 | rs9405090 | 32,406,350 | C6orf10 | 3.32E−15 | G | 0.40 | 0.52 | 1.53 | 1.38 | 1.70 |
| 6 | rs1003878 | 32,407,800 | C6orf10 | 1.90E−10 | A | 0.22 | 0.13 | 0.61 | 0.53 | 0.71 |
| 6 | rs1033500 | 32,415,360 | C6orf10 | 4.63E−15 | A | 0.40 | 0.52 | 1.53 | 1.38 | 1.69 |
| 6 | rs2076537 | 32,425,613 | C6orf10 | 2.81E−11 | A | 0.36 | 0.26 | 0.67 | 0.60 | 0.75 |
| 6 | rs9268368 | 32,441,933 | C6orf10 | 3.16E−15 | G | 0.40 | 0.52 | 1.53 | 1.38 | 1.70 |
| 6 | rs9268384 | 32,444,564 | C6orf10 | 3.41E−15 | G | 0.40 | 0.52 | 1.53 | 1.38 | 1.70 |
| 6 | rs3129939 | 32,444,744 | C6orf10 | 3.35E−11 | G | 0.17 | 0.09 | 0.53 | 0.45 | 0.64 |
| 6 | rs3129943 | 32,446,673 | C6orf10 | 1.06E−13 | G | 0.24 | 0.15 | 0.58 | 0.50 | 0.66 |
| 6 | rs4424066 | 32,462,406 | BTNL2 | 4.84E−12 | G | 0.42 | 0.51 | 1.44 | 1.30 | 1.59 |
| 6 | rs3117099 | 32,466,248 | BTNL2 | 2.11E−26 | A | 0.21 | 0.09 | 0.41 | 0.35 | 0.48 |
| 6 | rs3817973 | 32,469,089 | BTNL2 | 3.43E−12 | A | 0.42 | 0.51 | 1.44 | 1.30 | 1.59 |
| 6 | rs1980493 | 32,471,193 | BTNL2 | 8.63E−16 | G | 0.15 | 0.06 | 0.43 | 0.36 | 0.53 |
| 6 | rs2076530 | 32,471,794 | BTNL2 | 1.08E−10 | G | 0.42 | 0.51 | 1.40 | 1.27 | 1.55 |
| 6 | rs10947262 | 32,481,290 | BTNL2 | 6.01E−11 | A | 0.08 | 0.04 | 0.45 | 0.35 | 0.57 |
| 6 | rs3763309 | 32,483,951 | BTNL2 | 1.60E−15 | A | 0.20 | 0.30 | 1.60 | 1.43 | 1.79 |
| 6 | rs3763312 | 32,484,326 | BTNL2 | 2.53E−16 | A | 0.20 | 0.30 | 1.62 | 1.44 | 1.82 |
| 6 | rs3129963 | 32,488,186 | BTNL2 | 2.16E−19 | G | 0.17 | 0.07 | 0.42 | 0.35 | 0.50 |
| 6 | rs6932542 | 32,488,240 | BTNL2 | 2.34E−06 | A | 0.49 | 0.42 | 0.78 | 0.70 | 0.86 |
| 6 | rs9268528 | 32,491,086 | BTNL2 | 1.25E−21 | G | 0.37 | 0.51 | 1.68 | 1.51 | 1.87 |
| 6 | rs9268530 | 32,491,201 | BTNL2 | 9.00E−19 | G | 0.16 | 0.07 | 0.41 | 0.34 | 0.50 |
| 6 | rs9268542 | 32,492,699 | BTNL2 | 2.67E−20 | G | 0.38 | 0.51 | 1.65 | 1.49 | 1.83 |
| 6 | rs2395162 | 32,495,758 | BTNL2 | 4.55E−19 | A | 0.16 | 0.07 | 0.41 | 0.34 | 0.50 |
| 6 | rs2395163 | 32,495,787 | BTNL2 | 1.51E−11 | G | 0.20 | 0.28 | 1.50 | 1.34 | 1.69 |
| 6 | rs3135353 | 32,500,855 | HLA-DRA | 6.49E−15 | A | 0.14 | 0.06 | 0.43 | 0.35 | 0.52 |
| 6 | rs9268615 | 32,510,867 | HLA-DRA | 1.22E−25 | A | 0.39 | 0.54 | 1.75 | 1.58 | 1.94 |
| 6 | rs2395173 | 32,512,837 | HLA-DRA | 1.04E−05 | A | 0.34 | 0.29 | 0.78 | 0.70 | 0.87 |
| 6 | rs2395174 | 32,512,856 | HLA-DRA | 1.11E−11 | C | 0.28 | 0.18 | 0.63 | 0.56 | 0.72 |
| 6 | rs2395175 | 32,513,004 | HLA-DRA | 2.25E−12 | A | 0.14 | 0.21 | 1.61 | 1.41 | 1.84 |
| 6 | rs3129871 | 32,514,320 | HLA-DRA | 2.02E−08 | A | 0.36 | 0.29 | 0.73 | 0.66 | 0.81 |
| 6 | rs2239804 | 32,519,501 | HLA-DRA | 5.03E−28 | A | 0.54 | 0.38 | 0.55 | 0.49 | 0.61 |
| 6 | rs7192 | 32,519,624 | HLA-DRA | 2.93E−31 | A | 0.39 | 0.23 | 0.49 | 0.44 | 0.56 |
| 6 | rs2395182 | 32,521,295 | HLA-DRA | 5.56E−08 | C | 0.22 | 0.17 | 0.69 | 0.61 | 0.79 |
| 6 | rs3129890 | 32,522,251 | HLA-DRA | 7.00E−19 | G | 0.26 | 0.15 | 0.53 | 0.46 | 0.61 |
| 6 | rs9268832 | 32,535,767 | HLA-DRA | 9.03E−23 | A | 0.40 | 0.27 | 0.56 | 0.50 | 0.63 |
| 6 | rs2187668 | 32,713,862 | HLA-DQA1 | 4.01E−08 | A | 0.11 | 0.06 | 0.54 | 0.44 | 0.66 |
| 6 | rs1063355 | 32,735,692 | HLA-DQA1 | 2.46E−11 | A | 0.42 | 0.34 | 0.69 | 0.62 | 0.77 |
| 6 | rs9275224 | 32,767,856 | HLA-DQA1 | 3.60E−17 | A | 0.49 | 0.37 | 0.63 | 0.57 | 0.70 |
| 6 | rs6457617 | 32,771,829 | HLA-DQA2 | 8.75E−18 | G | 0.50 | 0.37 | 0.62 | 0.56 | 0.69 |
| 6 | rs2647012 | 32,772,436 | HLA-DQA2 | 1.69E−29 | A | 0.39 | 0.23 | 0.50 | 0.45 | 0.56 |
| 6 | rs9357152 | 32,772,938 | HLA-DQA2 | 4.65E−26 | G | 0.26 | 0.39 | 1.81 | 1.62 | 2.01 |
| 6 | rs1794282 | 32,774,504 | HLA-DQA2 | 5.99E−08 | A | 0.10 | 0.04 | 0.50 | 0.40 | 0.63 |
| 6 | rs2856725 | 32,774,716 | HLA-DQA2 | 7.28E−30 | G | 0.39 | 0.23 | 0.50 | 0.44 | 0.56 |
| 6 | rs11752643 | 32,777,351 | HLA-DQA2 | 6.52E−10 | A | 0.03 | 0.01 | 0.18 | 0.10 | 0.33 |
| 6 | rs2647050 | 32,777,745 | HLA-DQA2 | 6.94E−32 | G | 0.37 | 0.53 | 1.87 | 1.69 | 2.07 |
| 6 | rs2856718 | 32,778,233 | HLA-DQA2 | 7.36E−32 | A | 0.37 | 0.53 | 1.87 | 1.69 | 2.07 |
| 6 | rs2856717 | 32,778,286 | HLA-DQA2 | 1.47E−28 | A | 0.38 | 0.23 | 0.51 | 0.45 | 0.57 |
| 6 | rs2858305 | 32,778,442 | HLA-DQA2 | 1.67E−28 | C | 0.39 | 0.23 | 0.51 | 0.45 | 0.57 |
| 6 | rs16898264 | 32,785,130 | HLA-DQA2 | 1.66E−32 | A | 0.37 | 0.53 | 1.88 | 1.70 | 2.09 |
| 6 | rs9275572 | 32,786,977 | HLA-DQA2 | 1.38E−35 | A | 0.41 | 0.24 | 0.47 | 0.42 | 0.53 |
| 6 | rs7745656 | 32,788,948 | HLA-DQA2 | 6.71E−17 | A | 0.29 | 0.40 | 1.59 | 1.43 | 1.77 |
| 6 | rs2858332 | 32,789,139 | HLA-DQA2 | 2.46E−16 | C | 0.49 | 0.37 | 0.64 | 0.57 | 0.71 |
| 6 | rs2858331 | 32,789,255 | HLA-DQA2 | 2.70E−14 | G | 0.41 | 0.52 | 1.51 | 1.36 | 1.67 |
| 6 | rs3104404 | 32,790,152 | HLA-DQA2 | 5.54E−08 | A | 0.20 | 0.27 | 1.40 | 1.24 | 1.58 |
| 6 | rs3104405 | 32,790,286 | HLA-DQA2 | 2.51E−08 | C | 0.32 | 0.26 | 0.72 | 0.64 | 0.81 |
| 6 | rs12177980 | 32,794,062 | HLA-DQA2 | 5.05E−14 | A | 0.41 | 0.52 | 1.49 | 1.35 | 1.65 |
| 6 | rs9275659 | 32,794,081 | HLA-DQA2 | 2.63E−11 | A | 0.20 | 0.12 | 0.59 | 0.51 | 0.69 |
| 6 | rs9275686 | 32,795,548 | HLA-DQA2 | 1.96E−11 | A | 0.20 | 0.12 | 0.59 | 0.51 | 0.69 |
| 6 | rs9275698 | 32,795,951 | HLA-DQA2 | 8.70E−08 | G | 0.35 | 0.27 | 0.73 | 0.65 | 0.81 |
| 6 | rs9461799 | 32,797,507 | HLA-DQA2 | 6.01E−14 | G | 0.41 | 0.52 | 1.49 | 1.35 | 1.65 |
| 6 | rs2859078 | 32,810,427 | HLA-DQA2 | 9.09E−12 | G | 0.21 | 0.13 | 0.59 | 0.51 | 0.69 |
| 6 | rs13199787 | 32,813,254 | HLA-DQA2 | 8.76E−13 | A | 0.42 | 0.52 | 1.46 | 1.32 | 1.62 |
| 6 | rs17500468 | 32,819,156 | HLA-DQA2 | 1.18E−07 | G | 0.13 | 0.18 | 1.45 | 1.27 | 1.67 |
| 6 | rs9276435 | 32,821,845 | HLA-DQA2 | 1.85E−08 | A | 0.17 | 0.10 | 0.62 | 0.53 | 0.72 |
| 6 | rs2071800 | 32,822,121 | HLA-DQA2 | 1.06E−09 | A | 0.07 | 0.11 | 1.71 | 1.44 | 2.03 |
| 6 | rs10807113 | 32,830,164 | HLA-DQB2 | 8.02E−10 | C | 0.50 | 0.41 | 0.72 | 0.65 | 0.80 |
| 6 | rs7756516 | 32,831,895 | HLA-DQB2 | 6.59E−10 | G | 0.50 | 0.41 | 0.72 | 0.65 | 0.79 |
| 6 | rs2301271 | 32,833,171 | HLA-DQB2 | 1.04E−11 | A | 0.42 | 0.32 | 0.68 | 0.61 | 0.76 |
| 6 | rs7453920 | 32,837,990 | HLA-DQB2 | 1.79E−11 | A | 0.42 | 0.32 | 0.69 | 0.62 | 0.76 |
| 6 | rs2051549 | 32,838,064 | HLA-DQB2 | 6.30E−13 | G | 0.42 | 0.31 | 0.66 | 0.60 | 0.74 |
| 6 | rs2071550 | 32,838,918 | HLA-DQB2 | 6.73E−05 | A | 0.32 | 0.38 | 1.25 | 1.12 | 1.38 |
| 6 | rs6903130 | 32,840,188 | HLA-DQB2 | 1.73E−13 | G | 0.50 | 0.39 | 0.68 | 0.61 | 0.75 |
| 6 | rs6901084 | 32,844,914 | HLA-DQB2 | 3.27E−10 | A | 0.44 | 0.54 | 1.40 | 1.26 | 1.55 |
| 6 | rs9368741 | 32,845,485 | HLA-DQB2 | 7.15E−05 | A | 0.32 | 0.38 | 1.25 | 1.12 | 1.38 |
| 6 | rs9276644 | 32,853,021 | HLA-DQB2 | 1.60E−05 | G | 0.34 | 0.39 | 1.26 | 1.14 | 1.39 |
| 6 | rs7758736 | 32,866,372 | HLA-DOB | 2.07E−08 | A | 0.17 | 0.10 | 0.62 | 0.53 | 0.73 |
| 6 | rs3948793 | 32,867,426 | HLA-DOB | 8.17E−05 | A | 0.35 | 0.39 | 1.23 | 1.11 | 1.37 |
| 6 | rs17429444 | 32,894,046 | HLA-DOB | 3.93E−07 | G | 0.11 | 0.16 | 1.46 | 1.26 | 1.69 |
| 6 | rs3819721 | 32,912,776 | TAP2 | 6.42E−06 | A | 0.23 | 0.29 | 1.30 | 1.16 | 1.46 |
| 6 | rs1480380 | 33,021,224 | HLA-DMA | 4.66E−05 | A | 0.08 | 0.04 | 0.60 | 0.47 | 0.75 |
| 6 | rs1476387 | 109,871,228 | SMPD2 | 5.11E−05 | A | 0.42 | 0.48 | 1.23 | 1.12 | 1.36 |
| 6 | rs2025148 | 110,134,636 | KIAA0274 | 7.38E−05 | A | 0.39 | 0.44 | 1.23 | 1.11 | 1.36 |
| 6 | rs2343266 | 150,371,754 | RAET1L | 3.14E−05 | G | 0.19 | 0.23 | 1.29 | 1.15 | 1.46 |
| 6 | rs12209388 | 150,390,825 | RAET1L | 9.85E−07 | G | 0.20 | 0.25 | 1.34 | 1.20 | 1.51 |
| 6 | rs12183587 | 150,396,301 | RAET1L | 2.01E−18 | C | 0.43 | 0.32 | 0.62 | 0.56 | 0.69 |
| 6 | rs1413901 | 150,397,134 | RAET1L | 2.76E−08 | G | 0.12 | 0.17 | 1.50 | 1.30 | 1.72 |
| 6 | rs6935051 | 150,398,646 | RAET1L | 2.92E−08 | G | 0.38 | 0.45 | 1.33 | 1.21 | 1.47 |
| 6 | rs9479482 | 150,399,705 | RAET1L | 4.49E−19 | G | 0.43 | 0.32 | 0.62 | 0.55 | 0.68 |
| 6 | rs644866 | 150,405,702 | RAET1L | 8.29E−06 | G | 0.18 | 0.23 | 1.32 | 1.17 | 1.49 |
| 6 | rs11155700 | 150,409,957 | ULBP3 | 7.10E−09 | G | 0.26 | 0.33 | 1.38 | 1.24 | 1.53 |
| 6 | rs12213837 | 150,410,656 | ULBP3 | 9.18E−09 | A | 0.26 | 0.33 | 1.38 | 1.24 | 1.53 |
| 6 | rs13729 | 150,424,186 | ULBP3 | 2.63E−10 | G | 0.27 | 0.35 | 1.41 | 1.27 | 1.57 |
| 6 | rs2010259 | 150,427,168 | ULBP3 | 2.04E−12 | A | 0.37 | 0.28 | 0.67 | 0.60 | 0.75 |
| 6 | rs12202737 | 150,429,439 | ULBP3 | 5.12E−10 | A | 0.28 | 0.35 | 1.41 | 1.27 | 1.57 |
| 6 | rs2009345 | 150,431,441 | ULBP3 | 4.43E−17 | G | 0.39 | 0.50 | 1.55 | 1.40 | 1.72 |
| 6 | rs470138 | 150,443,878 | ULBP3 | 2.19E−07 | C | 0.40 | 0.34 | 0.76 | 0.68 | 0.84 |
| 6 | rs9397624 | 150,447,389 | ULBP3 | 3.28E−07 | A | 0.40 | 0.34 | 0.76 | 0.69 | 0.84 |
| 6 | rs11759611 | 150,453,135 | ULBP3 | 2.05E−09 | C | 0.36 | 0.29 | 0.71 | 0.64 | 0.80 |
| 6 | rs9458348 | 162,069,529 | PARK2 | 8.79E−05 | G | 0.27 | 0.32 | 1.25 | 1.12 | 1.39 |
| 7 | rs847440 | 16,984,957 | BCMP11 | 5.37E−05 | A | 0.44 | 0.50 | 1.23 | 1.12 | 1.36 |
| 7 | rs4722166 | 22,705,287 | IL6 | 7.98E−05 | C | 0.34 | 0.39 | 1.24 | 1.12 | 1.38 |
| 7 | rs7776857 | 22,721,293 | IL6 | 7.72E−05 | C | 0.32 | 0.37 | 1.24 | 1.12 | 1.38 |
| 7 | rs10488223 | 132,436,737 | CHCHD3 | 3.07E−05 | G | 0.06 | 0.03 | 0.56 | 0.43 | 0.73 |
| 8 | rs10104470 | 3,022,070 | CSMD1 | 8.65E−05 | C | 0.43 | 0.49 | 1.23 | 1.11 | 1.35 |
| 8 | rs13257028 | 68,746,276 | CPA6 | 9.50E−05 | G | 0.35 | 0.39 | 1.24 | 1.11 | 1.37 |
| 8 | rs2553650 | 68,775,254 | CPA6 | 2.02E−05 | G | 0.27 | 0.31 | 1.28 | 1.15 | 1.43 |
| 9 | rs1997368 | 101,753,401 | STX17 | 5.44E−07 | G | 0.31 | 0.38 | 1.32 | 1.18 | 1.46 |
| 9 | rs10760706 | 101,763,513 | STX17 | 3.60E−07 | G | 0.31 | 0.38 | 1.32 | 1.19 | 1.47 |
| 9 | rs16918878 | 101,886,451 | TXNDC4 | 2.35E−05 | A | 0.27 | 0.33 | 1.27 | 1.14 | 1.41 |
| 10 | rs942201 | 6,126,298 | IL2RA | 5.89E−07 | A | 0.21 | 0.26 | 1.35 | 1.20 | 1.52 |
| 10 | rs1107345 | 6,127,301 | IL2RA | 4.48E−07 | A | 0.21 | 0.26 | 1.36 | 1.21 | 1.52 |
| 10 | rs706779 | 6,138,830 | IL2RA | 4.84E−08 | G | 0.49 | 0.42 | 0.75 | 0.68 | 0.83 |
| 10 | rs3118470 | 6,141,719 | IL2RA | 1.74E−12 | G | 0.30 | 0.38 | 1.48 | 1.33 | 1.65 |
| 10 | rs7072793 | 6,146,272 | IL2RA | 7.42E−07 | G | 0.40 | 0.46 | 1.30 | 1.18 | 1.44 |
| 10 | rs7073236 | 6,146,558 | IL2RA | 1.41E−06 | G | 0.40 | 0.46 | 1.29 | 1.17 | 1.43 |
| 10 | rs4147359 | 6,148,445 | IL2RA | 2.22E−08 | A | 0.33 | 0.39 | 1.36 | 1.23 | 1.51 |
| 10 | rs7090530 | 6,150,881 | IL2RA | 6.29E−05 | C | 0.42 | 0.38 | 0.81 | 0.73 | 0.89 |
| 10 | rs10905879 | 6,217,089 | RBM17 | 2.70E−06 | A | 0.17 | 0.22 | 1.35 | 1.20 | 1.53 |
| 10 | rs631902 | 6,269,580 | PFKFB3 | 8.59E−05 | A | 0.37 | 0.42 | 1.23 | 1.11 | 1.36 |
| 11 | rs694739 | 63,853,809 | PRDX5 | 4.14E−07 | G | 0.37 | 0.31 | 0.75 | 0.68 | 0.84 |
| 11 | rs538147 | 63,886,298 | RPS6KA4 | 2.96E−06 | A | 0.37 | 0.31 | 0.77 | 0.69 | 0.86 |
| 11 | rs645078 | 63,891,874 | RPS6KA4 | 2.38E−06 | C | 0.37 | 0.31 | 0.77 | 0.69 | 0.85 |
| 12 | rs2069408 | 54,650,588 | CDK2 | 1.75E−07 | G | 0.32 | 0.38 | 1.32 | 1.19 | 1.47 |
| 12 | rs11171710 | 54,654,345 | RAB5B | 3.06E−05 | A | 0.45 | 0.40 | 0.80 | 0.73 | 0.89 |
| 12 | rs773107 | 54,655,773 | RAB5B | 9.29E−08 | G | 0.32 | 0.39 | 1.33 | 1.20 | 1.47 |
| 12 | rs10876864 | 54,687,352 | SUOX | 8.41E−08 | G | 0.41 | 0.47 | 1.32 | 1.20 | 1.46 |
| 12 | rs1701704 | 54,698,754 | ZNFN1A4 | 3.21E−08 | C | 0.33 | 0.40 | 1.34 | 1.21 | 1.48 |
| 12 | rs705708 | 54,775,180 | ERBB3 | 1.27E−07 | A | 0.47 | 0.53 | 1.32 | 1.19 | 1.46 |
| 12 | rs10783779 | 54,778,147 | ERBB3 | 6.10E−07 | C | 0.41 | 0.47 | 1.30 | 1.18 | 1.44 |
| 12 | rs2069718 | 66,836,429 | IFNG | 1.55E−05 | A | 0.41 | 0.35 | 0.79 | 0.71 | 0.88 |
| 12 | rs4913277 | 66,868,439 | IL26 | 9.85E−05 | G | 0.39 | 0.33 | 0.81 | 0.73 | 0.90 |
| 12 | rs2870951 | 66,870,812 | IL26 | 7.18E−05 | A | 0.40 | 0.35 | 0.80 | 0.73 | 0.89 |
| 12 | rs2454722 | 121,737,171 | GPR109A | 6.87E−05 | G | 0.18 | 0.22 | 1.29 | 1.14 | 1.45 |
| 13 | rs9568142 | 48,467,070 | FNDC3A | 1.05E−04 | C | 0.04 | 0.06 | 1.58 | 1.26 | 1.98 |
| 13 | rs3895825 | 79,494,436 | SPRY2 | 1.00E−04 | C | 0.20 | 0.24 | 1.28 | 1.13 | 1.44 |
| 13 | rs7323548 | 112,320,879 | FLJ26443 | 2.52E−05 | A | 0.05 | 0.07 | 1.56 | 1.27 | 1.91 |
| 16 | rs17229044 | 10,970,437 | KIAA0350 | 9.98E−05 | A | 0.21 | 0.17 | 0.77 | 0.68 | 0.88 |
| 16 | rs12934193 | 11,011,226 | KIAA0350 | 2.75E−05 | G | 0.18 | 0.14 | 0.74 | 0.64 | 0.85 |
| 16 | rs12599402 | 11,097,389 | KIAA0350 | 1.03E−04 | G | 0.43 | 0.39 | 0.82 | 0.74 | 0.90 |
| 16 | rs998592 | 11,107,179 | KIAA0350 | 1.77E−05 | A | 0.43 | 0.37 | 0.80 | 0.72 | 0.88 |
| 16 | rs9933507 | 11,108,929 | KIAA0350 | 2.61E−05 | G | 0.43 | 0.38 | 0.80 | 0.73 | 0.89 |
| 16 | rs12103174 | 11,111,231 | KIAA0350 | 2.73E−05 | G | 0.43 | 0.38 | 0.80 | 0.73 | 0.89 |
| 16 | rs8060821 | 11,241,560 | SOCS1 | 1.16E−05 | C | 0.43 | 0.37 | 0.79 | 0.71 | 0.87 |
| 16 | rs408665 | 11,249,473 | SOCS1 | 2.30E−05 | A | 0.44 | 0.39 | 0.80 | 0.72 | 0.88 |
| 16 | rs243323 | 11,268,703 | TNP2 | 9.53E−05 | G | 0.32 | 0.28 | 0.80 | 0.71 | 0.89 |
| 16 | rs4451969 | 11,291,020 | PRM1 | 1.39E−05 | A | 0.36 | 0.30 | 0.78 | 0.70 | 0.87 |
| 16 | rs7203055 | 11,381,157 | MGC24665 | 5.79E−05 | G | 0.37 | 0.32 | 0.80 | 0.72 | 0.89 |
| 16 | rs7500151 | 84,115,480 | KIAA0182 | 7.50E−05 | A | 0.36 | 0.31 | 0.80 | 0.72 | 0.89 |
| 18 | rs9945360 | 9,138,164 | ANKRD12 | 5.47E−05 | A | 0.39 | 0.33 | 0.80 | 0.72 | 0.89 |
| 18 | rs4798791 | 9,245,982 | ANKRD12 | 3.59E−05 | A | 0.39 | 0.33 | 0.80 | 0.72 | 0.89 |
| 18 | rs1893217 | 12,799,340 | PTPN2 | 4.09E−06 | G | 0.16 | 0.20 | 1.36 | 1.20 | 1.55 |
| 19 | rs8106303 | 40,349,191 | FXYD5 | 1.06E−04 | A | 0.22 | 0.19 | 0.78 | 0.68 | 0.88 |
| 19 | rs12110 | 40,352,348 | FXYD5 | 9.16E−05 | G | 0.22 | 0.19 | 0.77 | 0.68 | 0.88 |
| 20 | rs2247082 | 1,601,863 | SIRPB2 | 9.29E−05 | A | 0.23 | 0.26 | 1.27 | 1.13 | 1.42 |
| 20 | rs2377318 | 29,916,695 | DUSP15 | 4.47E−05 | A | 0.29 | 0.34 | 1.25 | 1.13 | 1.40 |
| 21 | rs2825523 | 19,627,751 | PRSS7 | 1.81E−05 | A | 0.39 | 0.45 | 1.25 | 1.13 | 1.39 |

TABLE 3
Haplotype organization of SNPs that exceed genome-wide significance.

| | | | | | Risk Allele | | | | |
|---|---|---|---|---|---|---|---|---|---|
| | | Risk | | | Frequency | | Allelic χ² | | |
| Chr | Locus | Haplotype | Marker | Mb | Controls | Cases | pvalue | OR (95% CI) | GWAS |

Associations outside of the HLA

| | | | | | | | | | |
|---|---|---|---|---|---|---|---|---|---|
| 2q33 | CTLA4/ICOS | 2_1 | rs1024161 * | 204.43 | 0.40 | 0.49 | 3.55 × 10⁻¹³ | 1.44 (1.30-1.59) | |
| | | | rs926169 | 204.43 | 0.39 | 0.47 | 5.50 × 10⁻¹¹ | 1.38 (1.25-1.52) | |
| | | | rs231726 | 204.45 | 0.32 | 0.39 | 1.94 × 10⁻¹⁰ | 1.38 (1.24-1.53) | T1D¹¹ |
| | | | rs231804 | 204.42 | 0.58 | 0.65 | 4.97 × 10⁻¹⁰ | 1.38 (1.25-1.53) | |
| | | | rs231735 | 204.40 | 0.52 | 0.60 | 5.75 × 10⁻¹⁰ | 1.37 (1.24-1.52) | RA¹³ |
| | | 2_2 | rs3096851 * | 204.47 | 0.31 | 0.37 | 3.58 × 10⁻⁰⁸ | 1.32 (1.19-1.46) | |
| | | | rs3116504 | 204.48 | 0.31 | 0.37 | 3.73 × 10⁻⁰⁸ | 1.32 (1.19-1.46) | |
| | | | rs3096866 | 204.50 | 0.31 | 0.38 | 4.33 × 10⁻⁰⁸ | 1.32 (1.19-1.46) | |

Other autoimmune diseases associated with the region
Type I Diabetes, Rheumatoid Arthritis, Multiple Sclerosis, Thyroiditis,
Hashimoto diseases, Systemic Lupus Erythematosus, and Celiac Disease.

| | | | | | | | | |
|---|---|---|---|---|---|---|---|---|
| 4q26- | IL2/IL21 | 4_1 | rs7682241 * | 123.74 | 0.33 | 0.40 | 4.27 × 10⁻⁰⁸ | 1.34 (1.21-1.48) |
| q27 | | | rs2137497 | 123.78 | 0.39 | 0.46 | 5.34 × 10⁻⁰⁸ | 1.33 (1.20-1.46) |

Other autoimmune diseases associated with the region
Psoriasis, Type I Diabetes, Celiac Disease, Graves Disease, Rheumatoid Arthritis.

| | | | | | | | | |
|---|---|---|---|---|---|---|---|---|
| 6q25 | ULBP3/ULBP6 | 6_6 | rs9479482 * | 150.40 | 0.57 | 0.68 | 4.49 × 10⁻¹⁹ | 1.65 (1.48-1.83) |
| | | | rs12183587 | 150.40 | 0.57 | 0.68 | 2.01 × 10⁻¹⁸ | 1.63 (1.47-1.81) |
| | | | rs12202737 | 150.43 | 0.28 | 0.35 | 5.12 × 10⁻¹⁰ | 1.40 (1.26-1.55) |
| | | | rs11759611 | 150.45 | 0.64 | 0.71 | 2.05 × 10⁻⁰⁹ | 1.40 (1.26-1.56) |
| | | | rs12213837 | 150.41 | 0.26 | 0.33 | 9.18 × 10⁻⁰⁹ | 1.37 (1.23-1.52) |
| | | | rs470138 | 150.44 | 0.60 | 0.66 | 2.19 × 10⁻⁰⁷ | 1.31 (1.18-1.45) |
| | | 6_7 | rs2009345 * | 150.43 | 0.39 | 0.50 | 4.43 × 10⁻¹⁷ | 1.52 (1.38-1.68) |
| | | | rs2010259 | 150.43 | 0.63 | 0.72 | 2.04 × 10⁻¹² | 1.49 (1.33-1.66) |
| | | | rs13729 | 150.42 | 0.27 | 0.35 | 2.63 × 10⁻¹⁰ | 1.41 (1.27-1.56) |
| | | | rs11155700 | 150.41 | 0.26 | 0.33 | 7.10 × 10⁻⁰⁹ | 1.37 (1.23-1.53) |
| | | | rs1413901 | 150.40 | 0.12 | 0.17 | 2.76 × 10⁻⁰⁸ | 1.48 (1.29-1.70) |
| | | | rs6935051 | 150.40 | 0.38 | 0.45 | 2.92 × 10⁻⁰⁸ | 1.35 (1.22-1.49) |

Other autoimmune diseases associated with the region
none

| | | | | | | | | |
|---|---|---|---|---|---|---|---|---|
| 9q31.1 | STX17 | 9_1 | rs10760706 * | 101.76 | 0.31 | 0.38 | 3.60 × 10⁻⁷ | 1.32 (1.19-1.47) |

Other autoimmune diseases associated with the region
none

| | | | | | | | | | |
|---|---|---|---|---|---|---|---|---|---|
| 10p15- | IL2RA | 10_1 | rs4147359 * | 6.15 | 0.33 | 0.39 | 2.22 × 10⁻⁰⁸ | 1.30 (1.17-1.44) | SLE¹⁵ |
| p14 | | | rs706779 | 6.14 | 0.51 | 0.58 | 4.84 × 10⁻⁰⁸ | 1.29 (1.16-1.42) | |
| | | | rs1107345 | 6.13 | 0.21 | 0.26 | 4.48 × 10⁻⁰⁷ | 1.30 (1.16-1.46) | |
| | | 10_2 | rs3118470 * | 6.14 | 0.30 | 0.38 | 1.74 × 10⁻¹² | 1.41 (1.27-1.56) | |

Other autoimmune diseases associated with the region
Type I Diabetes, Multiple Sclerosis.

| | | | | | | | | |
|---|---|---|---|---|---|---|---|---|
| 11q13 | PRDX5 | 11_1 | rs694739 * | 63.85 | 0.63 | 0.69 | 4.14 × 10⁻⁰⁷ | 1.33 (1.19-1.48) |

Other autoimmune diseases associated with the region
Multiple Sclerosis.

| | | | | | | | | | |
|---|---|---|---|---|---|---|---|---|---|
| 12q13 | Eos (IKZF4) | 12_1 | rs1701704 * | 54.70 | 0.33 | 0.40 | 3.21 × 10⁻⁰⁸ | 1.34 (1.21-1.48) | T1D¹⁰ |
| | | | rs10876864 | 54.69 | 0.41 | 0.47 | 8.41 × 10⁻⁰⁸ | 1.32 (1.20-1.46) | |
| | | | rs773107 | 54.66 | 0.32 | 0.39 | 9.29 × 10⁻⁰⁸ | 1.33 (1.20-1.47) | |
| | | | rs2069408 | 54.65 | 0.32 | 0.38 | 1.75 × 10⁻⁰⁷ | 1.32 (1.19-1.47) | |
| | | 12_2 | rs705708 * | 54.78 | 0.47 | 0.53 | 1.27 × 10⁻⁰⁷ | 1.32 (1.19-1.46) | |

Other autoimmune diseases associated with the region
Type I Diabetes, Systemic Lupus Erythematosus.
HLA Associations

| | | | | | | | | | |
|---|---|---|---|---|---|---|---|---|---|
| 6p21.3 | HLA | 6_1 | rs9275572 * | 32.79 | 0.59 | 0.76 | 1.38 × 10⁻³⁵ | 2.21 (1.98-2.47) | MS¹¹ |
| | | | rs2647050 | 32.78 | 0.37 | 0.53 | 6.94 × 10−32 | 1.93 (1.75-2.14) | |
| | | | rs7192 | 32.52 | 0.61 | 0.77 | 2.93 × 10−31 | 2.12 (1.90-2.38) | RA^{3, 14}, |
| | | | | | | | | | SLE¹⁵ |
| | | | rs2647012 | 32.77 | 0.61 | 0.77 | 1.69 × 10−29 | 2.09 (1.87-2.34) | RA³ |
| | | | rs2856717 | 32.78 | 0.62 | 0.77 | 1.47 × 10−28 | 2.07 (1.85-2.32) | |
| | | | rs2239804 | 32.52 | 0.46 | 0.62 | 5.03 × 10−28 | 1.92 (1.74-2.12) | T1D¹⁰ |
| | | | rs3117099 | 32.47 | 0.79 | 0.91 | 2.11 × 10−26 | 2.55 (2.18-2.98) | |
| | | | rs9357152 | 32.77 | 0.26 | 0.39 | 4.65 × 10−26 | 1.84 (1.66-2.04) | CeD¹⁷, |
| | | | | | | | | | PBC¹⁸, |
| | | | | | | | | | T1D¹⁰ |
| | | | rs9268832 | 32.54 | 0.60 | 0.73 | 9.03 × 10−23 | 1.87 (1.68-2.09) | |
| | | | rs9268528 | 32.49 | 0.37 | 0.51 | 1.25 × 10−21 | 1.75 (1.59-1.93) | T1D¹⁰ |
| | | | rs9268542 | 32.49 | 0.38 | 0.51 | 2.67 × 10−20 | 1.73 (1.56-1.91) | RA¹⁴, |
| | | | | | | | | | T1D¹⁰ |
| | | | rs3129963 | 32.49 | 0.83 | 0.93 | 2.16 × 10−19 | 2.65 (2.22-3.16) | |
| | | | rs2395162 | 32.50 | 0.84 | 0.93 | 4.55 × 10−19 | 2.70 (2.25-3.23) | |
| | | | rs6457617 | 32.77 | 0.50 | 0.63 | 8.75 × 10−18 | 1.67 (1.51-1.85) | RA^{11, 12} |
| | | | rs6935269 | 32.37 | 0.78 | 0.89 | 1.45 × 10−16 | 2.15 (1.86-2.49) | |
| | | | rs6457536 | 32.38 | 0.79 | 0.89 | 8.44 × 10−16 | 2.14 (1.84-2.48) | |
| | | | rs3763309 | 32.48 | 0.20 | 0.30 | 1.60 × 10−15 | 1.67 (1.50-1.87) | |
| | | | rs547261 | 32.39 | 0.40 | 0.52 | 1.73 × 10−15 | 1.63 (1.47-1.79) | |
| | | | rs9268368 | 32.44 | 0.40 | 0.52 | 3.16 × 10−15 | 1.62 (1.46-1.78) | |
| | | | rs9405090 | 32.41 | 0.40 | 0.52 | 3.32 × 10−15 | 1.61 (1.46-1.78) | |
| | | | rs9368713 | 32.41 | 0.40 | 0.52 | 4.92 × 10−15 | 1.61 (1.46-1.78) | |
| | | | rs3135353 | 32.50 | 0.86 | 0.94 | 6.49 × 10−15 | 2.62 (2.15-3.19) | T1D¹⁰ |
| | | | rs547077 | 32.40 | 0.40 | 0.52 | 7.25 × 10−15 | 1.61 (1.45-1.77) | |
| | | | rs2858331 | 32.79 | 0.41 | 0.52 | 2.70 × 10−14 | 1.54 (1.39-1.70) | T1D¹⁰ |
| | | | rs3129943 | 32.45 | 0.76 | 0.85 | 1.06 × 10−13 | 1.90 (1.66-2.17) | T1D¹⁰ |
| | | | rs2395175 | 32.51 | 0.14 | 0.21 | 2.25 × 10−12 | 1.58 (1.40-1.80) | T1D¹⁰ |
| | | | rs4424066 | 32.46 | 0.42 | 0.51 | 4.84 × 10−12 | 1.48 (1.34-1.63) | T1D¹⁰ |
| | | | rs2301271 | 32.83 | 0.58 | 0.68 | 1.04 × 10−11 | 1.55 (1.39-1.71) | T1D¹⁰ |
| | | | rs707928 | 31.85 | 0.67 | 0.76 | 1.42 × 10−11 | 1.57 (1.41-1.76) | T1D¹⁰ |
| | | | rs3115573 | 32.33 | 0.44 | 0.54 | 2.63 × 10−11 | 1.53 (1.38-1.68) | |
| | | | rs2076537 | 32.43 | 0.64 | 0.74 | 2.81 × 10−11 | 1.61 (1.44-1.79) | T1D¹⁰ |
| | | | rs405875 | 32.32 | 0.44 | 0.54 | 3.94 × 10−11 | 1.52 (1.38-1.68) | |
| | | | rs10947262 | 32.48 | 0.92 | 0.96 | 6.01 × 10−11 | 2.18 (1.72-2.76) | |
| | | | rs6941112 | 32.05 | 0.33 | 0.42 | 7.50 × 10−11 | 1.52 (1.37-1.68) | |
| | | | rs11752643 | 32.78 | 0.97 | 0.99 | 6.52 × 10−10 | 5.43 (3.03-9.75) | |
| | | | rs2269426 | 32.18 | 0.40 | 0.50 | 7.08 × 10−10 | 1.46 (1.32-1.61) | T1D¹⁰ |
| | | | rs377763 | 32.31 | 0.79 | 0.86 | 1.03 × 10−08 | 1.61 (1.40-1.84) | T1D¹⁰ |
| | | | rs9276435 | 32.82 | 0.83 | 0.90 | 1.85 × 10−08 | 1.75 (1.50-2.04) | |
| | | | rs8111 | 32.19 | 0.29 | 0.38 | 2.01 × 10−08 | 1.45 (1.31-1.61) | |
| | | | rs3129871 | 32.51 | 0.64 | 0.71 | 2.02 × 10−08 | 1.38 (1.24-1.54) | |
| | | | rs7758736 | 32.87 | 0.83 | 0.90 | 2.07 × 10−08 | 1.72 (1.48-2.01) | T1D¹⁰ |
| | | | rs3104405 | 32.79 | 0.68 | 0.74 | 2.51 × 10−08 | 1.39 (1.25-1.56) | |
| | | | rs2395182 | 32.52 | 0.78 | 0.83 | 5.56 × 10−08 | 1.44 (1.27-1.64) | T1D¹⁰ |
| | | | rs7775397 | 32.37 | 0.90 | 0.95 | 5.91 × 10−08 | 2.36 (1.89-2.94) | T1D¹⁰ |
| | | | rs9275698 | 32.80 | 0.65 | 0.73 | 8.70 × 10−08 | 1.42 (1.27-1.58) | |
| | | | rs17500468 | 32.82 | 0.13 | 0.18 | 1.18 × 10−07 | 1.48 (1.29-1.69) | T1D¹⁰ |
| | | | rs805303 | 31.72 | 0.64 | 0.71 | 1.91 × 10−07 | 1.40 (1.25-1.55) | T1D¹⁰ |
| | | | rs494620 | 31.95 | 0.43 | 0.51 | 3.72 × 10−07 | 1.41 (1.28-1.56) | |
| | | 6_2 | rs16898264 * | 32.79 | 0.37 | 0.53 | 1.66 × 10−32 | 1.95 (1.77-2.16) | T1D¹⁰ |
| | | | rs2856718 | 32.78 | 0.37 | 0.53 | 7.36 × 10−32 | 1.94 (1.75-2.14) | T1D¹⁰ |
| | | | rs2856725 | 32.77 | 0.61 | 0.77 | 7.28 × 10−30 | 2.11 (1.88-2.36) | |
| | | | rs2858305 | 32.78 | 0.62 | 0.77 | 1.67 × 10−28 | 2.07 (1.85-2.32) | |
| | | | rs9268615 | 32.51 | 0.39 | 0.54 | 1.22 × 10−25 | 1.85 (1.67-2.04) | T1D¹⁰ |
| | | | rs3129890 | 32.52 | 0.74 | 0.85 | 7.00 × 10−19 | 1.97 (1.73-2.25) | |
| | | | rs9268530 | 32.49 | 0.84 | 0.93 | 9.00 × 10−19 | 2.68 (2.24-3.22) | T1D¹⁰ |
| | | | rs7745656 | 32.79 | 0.29 | 0.40 | 6.71 × 10−17 | 1.62 (1.46-1.79) | RA |
| | | | rs3130340 | 32.35 | 0.78 | 0.89 | 1.42 × 10−16 | 2.15 (1.86-2.49) | T1D¹⁰ |
| | | | rs2858332 | 32.79 | 0.51 | 0.63 | 2.46 × 10−16 | 1.62 (1.46-1.79) | |
| | | | rs1980493 | 32.47 | 0.85 | 0.94 | 8.63 × 10−16 | 2.60 (2.15-3.14) | T1D¹⁰ |
| | | | rs1033500 | 32.42 | 0.40 | 0.52 | 4.63 × 10−15 | 1.61 (1.46-1.78) | |
| | | | rs12177980 | 32.79 | 0.41 | 0.52 | 5.05 × 10−14 | 1.54 (1.40-1.70) | T1D¹⁰ |
| | | | rs6903130 | 32.84 | 0.50 | 0.61 | 1.73 × 10−13 | 1.56 (1.41-1.73) | T1D¹⁰ |
| | | | rs2051549 | 32.84 | 0.58 | 0.69 | 6.30 × 10−13 | 1.60 (1.44-1.78) | T1D¹⁰ |
| | | | rs13199787 | 32.81 | 0.42 | 0.52 | 8.76 × 10−13 | 1.52 (1.38-1.68) | T1D¹⁰ |
| | | | rs3817973 | 32.47 | 0.42 | 0.51 | 3.43 × 10−12 | 1.48 (1.34-1.64) | T1D¹⁰ |
| | | | rs2859078 | 32.81 | 0.79 | 0.87 | 9.09 × 10−12 | 1.76 (1.53-2.03) | |
| | | | rs2395174 | 32.51 | 0.72 | 0.82 | 1.11 × 10−11 | 1.71 (1.51-1.93) | T1D¹⁰ |
| | | | rs1063355 | 32.74 | 0.58 | 0.66 | 2.46 × 10−11 | 1.40 (1.27-1.56) | T1D¹⁰ |
| | | | rs3130315 | 32.33 | 0.44 | 0.54 | 2.71 × 10−11 | 1.53 (1.38-1.68) | |
| | | | rs3129939 | 32.44 | 0.83 | 0.91 | 3.35 × 10−11 | 2.11 (1.79-2.49) | T1D¹⁰ |
| | | | rs6901084 | 32.84 | 0.44 | 0.54 | 3.27 × 10−10 | 1.47 (1.33-1.63) | T1D¹⁰ |
| | | | rs7756516 | 32.83 | 0.50 | 0.59 | 6.59 × 10−10 | 1.46 (1.33-1.62) | T1D¹⁰ |
| | | | rs10807113 | 32.83 | 0.50 | 0.59 | 8.02 × 10−10 | 1.46 (1.32-1.61) | T1D¹⁰ |
| | | | rs9267947 | 32.32 | 0.55 | 0.64 | 2.08 × 10−09 | 1.47 (1.33-1.63) | |
| | | | rs652888 | 31.96 | 0.80 | 0.87 | 2.58 × 10−08 | 1.72 (1.49-1.98) | T1D¹⁰ |
| | | | rs389883 | 32.06 | 0.71 | 0.79 | 9.05 × 10−08 | 1.54 (1.37-1.73) | |
| | | | rs2442749 | 31.46 | 0.72 | 0.78 | 1.19 × 10−07 | 1.44 (1.28-1.62) | T1D¹⁰ |
| | | | rs805294 | 31.80 | 0.65 | 0.72 | 3.67 × 10−07 | 1.39 (1.25-1.55) | T1D¹⁰ |
| | | | rs1270942 | 32.03 | 0.90 | 0.95 | 4.49 × 10−07 | 2.18 (1.76-2.70) | T1D¹⁰ |
| | | | rs389884 | 32.05 | 0.90 | 0.95 | 4.97 × 10−07 | 2.18 (1.76-2.71) | T1D¹⁰ |
| | | 6_3 | rs3130320 * | 32.33 | 0.64 | 0.77 | 5.64 × 10−19 | 1.88 (1.68-2.11) | |
| | | | rs9275224 | 32.77 | 0.51 | 0.63 | 3.60 × 10−17 | 1.65 (1.49-1.83) | |
| | | | rs3115553 | 32.35 | 0.78 | 0.89 | 1.49 × 10−16 | 2.15 (1.85-2.48) | T1D¹⁰ |
| | | | rs9268132 | 32.36 | 0.40 | 0.52 | 1.58 × 10−15 | 1.62 (1.47-1.79) | |
| | | | rs9268384 | 32.44 | 0.40 | 0.52 | 3.41 × 10−15 | 1.62 (1.46-1.78) | |
| | | | rs9461799 | 32.80 | 0.41 | 0.52 | 6.01 × 10−14 | 1.54 (1.40-1.70) | T1D¹⁰ |
| | | | rs7453920 | 32.84 | 0.58 | 0.68 | 1.79 × 10−11 | 1.54 (1.39-1.71) | T1D¹⁰ |
| | | | rs9275686 | 32.80 | 0.80 | 0.88 | 1.96 × 10−11 | 1.78 (1.54-2.05) | |
| | | | rs9275659 | 32.79 | 0.80 | 0.88 | 2.63 × 10−11 | 1.77 (1.54-2.04) | |
| | | | rs2076530 | 32.47 | 0.42 | 0.51 | 1.08 × 10−10 | 1.45 (1.31-1.60) | T1D¹⁰ |
| | | | rs1003878 | 32.41 | 0.78 | 0.87 | 1.90 × 10−10 | 1.81 (1.58-2.08) | T1D¹⁰ |
| | | | rs2072633 | 32.03 | 0.58 | 0.67 | 3.60 × 10−10 | 1.51 (1.36-1.68) | |
| | | | rs4151657 | 32.03 | 0.36 | 0.44 | 3.21 × 10−08 | 1.43 (1.30-1.58) | |
| | | | rs2187668 | 32.71 | 0.89 | 0.94 | 4.01 × 10−08 | 2.15 (1.76-2.62) | CeD¹⁷, |
| | | | | | | | | | SLE¹⁵ |
| | | | rs1077393 | 31.72 | 0.50 | 0.58 | 5.89 × 10−08 | 1.39 (1.26-1.54) | T1D¹⁰ |
| | | | rs1794282 | 32.77 | 0.90 | 0.96 | 5.99 × 10−08 | 2.36 (1.89-2.95) | T1D¹⁰ |
| | | | rs437179 | 32.04 | 0.71 | 0.79 | 8.48 × 10−08 | 1.54 (1.37-1.73) | |
| | | | rs6911628 | 30.85 | 0.73 | 0.81 | 2.80 × 10−07 | 1.50 (1.32-1.69) | T1D¹⁰ |
| | | 6_4 | rs3763312 * | 32.48 | 0.20 | 0.30 | 2.53 × 10−16 | 1.70 (1.52-1.89) | T1D¹⁰ |
| | | | rs2070600 | 32.26 | 0.04 | 0.08 | 1.15 × 10−10 | 1.94 (1.59-2.37) | |
| | | | rs2071800 | 32.82 | 0.07 | 0.11 | 1.06 × 10−09 | 1.78 (1.51-2.10) | |
| | | 6_5 | rs6910071 * | 32.39 | 0.18 | 0.26 | 2.95 × 10−13 | 1.57 (1.40-1.76) | T1D¹⁰ |
| | | | rs2395163 | 32.50 | 0.20 | 0.28 | 1.51 × 10−11 | 1.56 (1.39-1.75) | T1D¹⁰ |
| | | | rs3104404 | 32.79 | 0.20 | 0.27 | 5.54 × 10−08 | 1.44 (1.28-1.61) | T1D¹⁰ |
| | | | rs17429444 | 32.89 | 0.11 | 0.16 | 3.93 × 10−07 | 1.53 (1.33-1.76) | |

* indicates marker is used as a proxy to represent the group of highly correlated SNPs. Type I diabetes (T1D), rheumatoid arthritis (RA), celiac disease (CeD), multiple sclerosis (MS), system lupus erythematosus (SLE), primary biliary cirrhosis (PBC).

TABLE 4
Immune related genes with nominal significance.

| | | Count of | Min | Min | | |
|---|---|---|---|---|---|---|
| | | SNPs < | p-value | p-value | Autoimmune | GO |
| Gene | Mb | 1 × 10⁻⁴ | observed | imputed | Reports | classification |
| Chromosome 2 | | | | | | |
| HDAC4 | 240.03 | 1 | 8.10E−05 | 5.59E−05 | | inflammatory |
| | | | | | | response |
| Chromosome 3 | | | | | | |
| CACNA2D3 | 55.02 | 1 | 7.28E−05 | 1.47E−05 | CeD | |
| Chromosome 5 | | | | | | |
| IL13 | 132.02 | 2 | 1.87E−06 | | Asthma | immune |
| | | | | | | response |
| Chromosome 6 | | | | | | |
| HLA-G | 29.94 | 1 | 1.07E−04 | 4.54E−06 | RA, MS, SLE, | immune |
| | | | | | PS, T1D, | response |
| | | | | | Asthma, | |
| HLA-A | 30.01 | 1 | 1.00E−04 | 2.72E−05 | MS, T1D, PS, | immune |
| | | | | | GD, | response |
| | | | | | Asthma, Vitilago | |
| MICB | 31.59 | 2 | 1.89E−05 | 1.97E−05 | MS, T1D, UC, | immune |
| | | | | | RA, CeD, | response |
| | | | | | Asthma | |
| TAP2 | 32.91 | 1 | 6.42E−06 | 1.28E−05 | T1D, RA, SLE, | immune |
| | | | | | PS, GD | response |
| Chromosome 7 | | | | | | |
| IL6 | 22.72 | 2 | 7.72E−05 | 4.84E−05 | RA, T1D, CeD | inflammatory |
| | | | | | | response |
| CHCHD3 | 132.44 | 1 | 3.07E−05 | 2.02E−05 | CeD | |
| Chromosome 8 | | | | | | |
| CSMD1 | 3.02 | 1 | 8.65E−05 | 8.38E−05 | CeD, MS | |
| Chromosome 12 | | | | | | |
| IFNG | 66.84 | 1 | 1.55E−05 | 1.29E−05 | CeD, T1D, RA, | |
| | | | | | MS, SLE, PS, | |
| | | | | | GD, Asthma | |
| IL26 | 66.87 | 2 | 7.18E−05 | 6.45E−05 | MS, Asthma | immune |
| | | | | | | response |
| Chromosome 16 | | | | | | |
| KIAA0350 | 11.11 | 6 | 1.77E−05 | 1.15E−05 | T1D, MS, | |
| (CLEC16A) | | | | | Thyroid | |
| | | | | | Disease | |
| SOCS1 | 11.24 | 2 | 1.16E−05 | 8.66E−06 | CeD, T1D, | |
| | | | | | Asthma | |
| Chromosome 18 | | | | | | |
| ANKRD12 | 9.25 | 2 | 3.59E−05 | 1.55E−05 | | |
| PTPN2 | 12.80 | 1 | 4.09E−06 | 3.38E−07 | CD, T1D | |

Celiac Disease (CeD), rheumatoid arthritis (RA), multiple sclerosis (MS), system lupus erythematosus (SLE), psoriasis (PS), type I diabetes (T1D), Graves disease (GD).

TABLE 5
Population Attributable Fractions.

| | | | | | 95% Wald | | | Population |
|---|---|---|---|---|---|---|---|---|
| | | Frequency | Percent | Odds | Confidence | | | Attributable |
| | | (control) | (control) | Ratio | Limits | | P-value | Fraction |
| Chromosome 2q33.2 | GG | 1149 | 35.84 | 1.00 | | | | |
| rs1024161 | AG | 1527 | 47.63 | 1.45 | 1.226 | 1.706 | <.0001 | |
| | AA | 530 | 16.53 | 2.06 | 1.685 | 2.509 | <.0001 | 27.90% |
| Chromosome 4q24 | CC | 1438 | 44.03 | 1.00 | | | | |
| rs7682241 | AC | 1468 | 44.95 | 1.22 | 1.048 | 1.421 | 0.0105 | |
| | AA | 360 | 11.02 | 1.90 | 1.540 | 2.344 | <.0001 | 16.53% |
| Chromosome 6p21.3 | AA | 571 | 17.44 | 1.00 | | | | |
| rs9275572 | AG | 1561 | 47.66 | 2.57 | 0.414 | 0.557 | <.0001 | |
| | GG | 1143 | 34.9 | 5.36 | 0.139 | 0.250 | <.0001 | 69.44% |
| Chromosome 6q25 | GG | 621 | 19.01 | 1.00 | | | | |
| rs9479482 | GA | 1587 | 48.59 | 1.57 | 0.516 | 0.696 | <.0001 | |
| | AA | 1058 | 32.39 | 2.62 | 0.303 | 0.479 | <.0001 | 44.56% |
| Chromosome 9q31.1 | AA | 1491 | 45.5 | 1.00 | | | | |
| rs1997368 | CA | 1411 | 43.06 | 1.38 | 1.182 | 1.600 | <.0001 | |
| | CC | 375 | 11.44 | 1.74 | 1.401 | 2.149 | <.0001 | 19.71% |
| Chromosome 10p15.1 | AA | 1528 | 46.66 | 1.00 | | | | |
| rs3118470 | GA | 1435 | 43.82 | 1.26 | 1.084 | 1.462 | 0.0026 | |
| | GG | 312 | 9.53 | 1.83 | 1.467 | 2.285 | <.0001 | 16.16% |
| Chromosome 11q13 | GG | 428 | 13.06 | 1.00 | | | | |
| rs694739 | GA | 1563 | 47.68 | 1.13 | 0.601 | 0.808 | 0.3001 | |
| | AA | 1287 | 39.26 | 1.63 | 0.485 | 0.778 | <.0001 | 23.69% |
| Chromosome 12q13 | AA | 1566 | 47.79 | 1.00 | | | | |
| rs1701704 | GA | 1441 | 43.97 | 1.35 | 1.166 | 1.572 | <.0001 | |
| | GG | 270 | 8.24 | 2.13 | 1.695 | 2.677 | <.0001 | 19.92% |

SNPs for which the major allele is associated with risk.

TABLE 6
Comparison of AA GWAS findings to published
AA candidate gene studies.

| | | | No. of | most signif- |
|---|---|---|---|---|
| Conclu- | | | published | icant SNP in |
| sion in | | | AA candidate | AA GWAS |
| Literature | | Gene | gene studies | (pvalue) |
| Asso- | non-HLA | PTPN22 | 2 | 1.98 × 10⁻⁴ |
| ciation | | FLG2 | 1 | 0.24 |
| | | IL1RN | 1 | 0.07 |
| | | MIF | 1 | 0.54 |
| | | NOS3 | 1 | 0.32 |
| | | AIRE* | 2 | 0.05 |
| | HLA | NOTCH4 | 1 | 1.03 × 10⁻⁸ |
| | | HLA-DRB1 | 5 | 9.03 × 10⁻²³ |
| | | HLA-A | 3 | 1.0 × 10⁻⁰⁴ |
| | | HLA-B | 3 | 0.05 |
| | | HLA-DQB1 | 3 | 2.46 × 10⁻¹¹ |
| | | HLA-C | 2 | 0.03 |
| | | MICA | 1 | 1.19 × 10⁻⁷ |
| | | HLA-DQA1 | 2 | 4.01 × 10⁻⁸ |
| No | HLA | VDR | 2 | 0.03 |
| Asso- | | FCRL3 | 1 | 0.13 |
| ciation | | IL1B | 1 | 0.05 |
| | | CCL2 | 1 | 0.37 |
| | | IL1A | 1 | 0.55 |
| | | AIRE* | 1 | 0.05 |

### REFERENCES

A1. Safavi K H, Muller S A, Suman V J, Moshell A N, Melton L J, 3rd. Incidence of alopecia areata in Olmsted County, Minnesota, 1975 through 1989. Mayo Clin Proc 1995; 70:628-33.
A2. Jelinek J E. Sudden whitening of the hair. Bull N Y Acad Med 1972; 48:1003-13.
A3. Ito T, Ito N, Saatoff M, et al. Maintenance of hair follicle immune privilege is linked to prevention of NK cell attack. The Journal of investigative dermatology 2008; 128:1196-206.
A4. Todes-Taylor N, Turner R, Wood G S, Stratte P T, Morhenn V B. T cell subpopulations in alopecia areata. J Am Acad Dermatol 1984; 1 1:216-23.
A5. Gilhar A, Landau M, Assy B, et al. Transfer of alopecia areata in the human scalp graft/Prkdc(scid) (SCID) mouse system is characterized by a TH1 response. Clin Immunol 2003; 106:181-7.
A6. Gilhar A, Shalaginov R, Assy B, Serafimovich S, Kalish R S. Alopecia areata is a T-lymphocyte mediated autoimmune disease: lesional human T-lymphocytes transfer alopecia areata to human skin grafts on SCID mice. J Investig Dermatol Symp Proc 1999; 4:207-10.
A7. Zoller M, McElwee K J, Engel P, Hoffmann R. Transient CD44 variant isoform expression and reduction in CD4(+)/CD25(+) regulatory T cells in C3H/HeJ mice with alopecia areata. The Journal of investigative dermatology 2002; 1 18:983-92.
A8. McElwee K J, Freyschmidt-Paul P, Hoffmann R, et al. Transfer of CD8(+) cells induces localized hair loss whereas CD4(+)/CD25(−) cells promote systemic alopecia areata and CD4(+)/CD25(+) cells blockade disease onset in the C3H/HeJ mouse model. The Journal of investigative dermatology 2005; 124:947-57.
A9. Ito T, Meyer K C, Ito N, Paus R. Immune privilege and the skin. Curr Dir Autoimmun 2008; 10:27-52.
A10. McDonagh A J, Tazi-Ahnini R. Epidemiology and genetics of alopecia areata. Clin Exp Dermatol 2002; 27:405-9.
A11. van der Steen P, Traupe H, Happle R, Boezeman J, Strater R, Hamm H. The genetic risk for alopecia areata in first degree relatives of severely affected patients. An estimate. Acta Derm Venereol 1992; 72:373-5.
A12. Jackow C, Puffer N, Hordinsky M, Nelson J, Tarrand J, Duvic M. Alopecia areata and cytomegalovirus infection in twins: genes versus environment? J Am Acad Dermatol 1998; 38:418-25.
A13. Martinez-Mir A, Zlotogorski A, Gordon D, et al. Genomewide scan for linkage reveals evidence of several susceptibility loci for alopecia areata. Am J Hum Genet. 2007; 80:316-28.
N1. Safavi, K. H., Muller, S. A., Suman, V. J., Moshell, A. N. & Melton, L. J., 3rd. Incidence of alopecia areata in Olmsted County, Minnesota, 1975 through 1989. Mayo Clin Proc 70, 628-33 (1995).
N2. Jelinek, J. E. Sudden whitening of the hair. Bull N Y Acad Med 48, 1003-13 (1972).
N3. Gilhar, A., Paus, R. & Kalish, R. S. Lymphocytes, neuropeptides, and genes involved in alopecia areata. J Clin Invest 117, 2019-27 (2007).
N4. Ito, T., Meyer, K. C., Ito, N. & Paus, R. Immune privilege and the skin. Curr Dir Autoimmun 10, 27-52 (2008).
N5. McDonagh, A. J. & Tazi-Ahnini, R. Epidemiology and genetics of alopecia areata. Clin Exp Dermatol 27, 405-9 (2002).
N6. van der Steen, P. et al. The genetic risk for alopecia areata in first degree relatives of severely affected patients. An estimate. Acta Derm Venereol 72, 373-5 (1992).
N7. Jackow, C. et al. Alopecia areata and cytomegalovirus infection in twins: genes versus environment? J Am Acad Dermatol 38, 418-25 (1998).
N8. Martinez-Mir, A. et al. Genomewide scan for linkage reveals evidence of several susceptibility loci for alopecia areata. Am J Hum Genet. 80, 316-28 (2007).
N9. Duvic, M., Norris, D., Christiano, A., Hordinsky, M. & Price, V. Alopecia areata registry: an overview. J Investig Dermatol Symp Proc 8, 219-21 (2003).
N10. Mitchell, M. K., Gregersen, P. K., Johnson, S., Parsons, R. & Vlahov, D. The New York Cancer Project: rationale, organization, design, and baseline characteristics. J Urban Health 81, 301-10 (2004).
N11. Hunter, D. J. et al. A genome-wide association study identifies alleles in FGFR2 associated with risk of sporadic postmenopausal breast cancer. Nat Genet. 39, 870-4 (2007).
N12. Yeager, M. et al. Genome-wide association study of prostate cancer identifies a second risk locus at 8q24. Nat Genet. 39, 645-9 (2007).
N13. Hindorff, L. A. et al. Potential etiologic and functional implications of genome-wide association loci for human diseases and traits. Proc Natl Acad Sci USA 106, 9362-7 (2009).
N14. Radosavljevic, M. et al. A cluster of ten novel MHC class I related genes on human chromosome 6q24.2-q25.3. Genomics 79, 114-23 (2002).
N15. Eagle, R. A. & Trowsdale, J. Promiscuity and the single receptor: NKG2D. Nat Rev Immunol 7, 737-44 (2007).
N16. Eagle, R. A., Traherne, J. A., Hair, J. R., Jafferji, I. & Trowsdale, J. ULBP6/RAET1L is an additional human NKG2D ligand. Eur J Immunol (2009).
N17. Caillat-Zucman, S. How NKG2D ligands trigger autoimmunity? Hum Immunol 67, 204-7 (2006).
N18. Strid, J. et al. Acute upregulation of an NKG2D ligand promotes rapid reorganization of a local immune compartment with pleiotropic effects on carcinogenesis. Nat Immunol 9, 146-54 (2008).
N19. Ito, T. et al. Maintenance of hair follicle immune privilege is linked to prevention of NK cell attack. J Invest Dermatol 128, 1196-206 (2008).
N20. Cetin, E. D., Savk, E., Uslu, M., Eskin, M. & Karul, A. Investigation of the inflammatory mechanisms in alopecia areata. Am J Dermatopathol 31, 53-60 (2009).
N21. Zhang, Q., Li, J., Deavers, M., Abbruzzese, J. L. & Ho, L. The subcellular localization of syntaxin 17 varies among different cell types and is altered in some malignant cells. J Histochem Cytochem 53, 1371-82 (2005).
N22. Rosengren Pielberg, G. et al. A cis-acting regulatory mutation causes premature hair graying and susceptibility to melanoma in the horse. Nat Genet. 40, 1004-9 (2008).
N23. Holley, J. E., Newcombe, J., Winyard, P. G. & Gutowski, N.J. Peroxiredoxin V in multiple sclerosis lesions: predominant expression by astrocytes. Mult Scler 13, 955-61 (2007).
N24. Colombe, L., Michelet, J. F. & Bernard, B. A. Prostanoid receptors in anagen human hair follicles. Exp Dermatol 17, 63-72 (2008).
N25. Libioulle, C. et al. Novel Crohn disease locus identified by genome-wide association maps to a gene desert on 5p13.1 and modulates expression of PTGER4. PLoS Genet. 3, e58 (2007).
N26. WTCCC. Genome-wide association study of 14,000 cases of seven common diseases and 3,000 shared controls. Nature 447, 661-678 (2007).
N27. De Jager, P. L. et al. Meta-analysis of genome scans and replication identify CD6, IRF8 and TNFRSF1A as new multiple sclerosis susceptibility loci. Nat Genet. 41, 776-82 (2009).
N28. Kabashima, K. et al. Prostaglandin E2-EP4 signaling initiates skin immune responses by promoting migration and maturation of Langerhans cells. Nat Med 9, 744-9 (2003).
N29. Pan, F. et al. Eos mediates Foxp3-dependent gene silencing in CD4+ regulatory T cells. Science 325, 1142-6 (2009).
N30. Wing, K. et al. CTLA-4 control over Foxp3+ regulatory T cell function. Science 322, 271-5 (2008).
N31. Monteleone, G., Pallone, F. & Macdonald, T. T. Interleukin-21 as a new therapeutic target for immune-mediated diseases. Trends Pharmacol Sci 30, 441-7 (2009).
N32. Gregersen, P. K. & Olsson, L. M. Recent advances in the genetics of autoimmune disease. Annu Rev Immunol 27, 363-91 (2009).
N33. Purcell, S. et al. PLINK: a tool set for whole-genome association and population-based linkage analyses. Am J Hum Genet. 81, 559-75 (2007).
N34. Tian, C. et al. European Population Genetic Substructure: Further Definition of Ancestry Informative Markers for Distinguishing Among Diverse European Ethnic Groups. Mol Med (2009).
N35. Barrett, J. C., Fry, B., Mailer, J. & Daly, M. J. Haploview: analysis and visualization of LD and haplotype maps. Bioinformatics 21, 263-5 (2005).
N36. Bazzi, H. et al. Desmoglein 4 is expressed in highly differentiated keratinocytes and trichocytes in human epidermis and hair follicle. Differentiation 74, 129-40 (2006).

### Example 2

### Study Samples, Genotyping, Quality Control and Population Stratification

### Study Samples

Cases were ascertained through the National Alopecia Areata Registry (NAAR) which recruits patients in the US primarily through five clinical sites.^{S1} In the course of enrollment, patients provided medical and family history as well as demographic information. Diagnosis was confirmed by clinical examiners prior to collecting blood samples. Written informed consent was obtained from all participants. The study was approved by the local IRB committees. In order to reduce the possibility of confounding from population stratification, only patients who self-reported European ancestry were selected for genotyping. Cases were genotyped with the Illumina 610K chip.

The control data used in the discovery GWAS was obtained from subjects enrolled in the New York Cancer Project^{S2} and genotyped as part of previous studies.^{S3}

For the replication data set, control data was obtained from the CGEMS breast^{S4} and prostate^{S5} cancer studies (http://cgems.cancer.gov/data/). The controls for the breast cancer arm of CGEMs were women from the Nurses Health Study^{S6} who were postmenopausal and had not diagnosed been with breast cancer during follow-up, and were matched to breast cancer cases based on age at diagnosis, blood collection variables (time of day, season, and year of blood collection, as well as recent (<3 months) use of postmenopausal hormones), ethnicity (all cases and controls are self-reported Caucasians), and menopausal status (all cases were postmenopausal at diagnosis).

Of the 1,184 controls that were originally genotyped, 1,142 controls met quality control requirements and have been distributed through the CGEMS portal. Genotyping of the CGEMS Breast Cancer Study was performed by the NCI Core Genotyping Facility using the Sentrix HumanHap550 genotyping assay. The controls for the prostate cancer arm of CGEMS were derived from participants in the PLCO trial and were matched via a density sampling procedure to cases. 1,204 different men, representing 1230 control selections, were identified as controls and were subsequently genotyped. Of these, 1094 passed quality control steps and have been made available for use by external investigators. Genotyping of the CGEMS Prostate Cancer Study was performed under contract by Illumina Corporation in two parts, Phase x1A used the Sentrix® HumanHap300 genotyping assay and Phase 1B used the Sentrix® HumanHap240.^{S7-S9} Of the 2358 individuals that were retained for previous analyses using CGEMS, 2243 were distributed via the CGEMs portal (http://cgems.cancer.gov/data/) for general analysis. Further filtering to remove individuals who had low call rate (<95%, 7 prostrate controls), leaving a total of 2236 combined breast and prostate controls for analysis.

Association Analysis.

Joint analysis of the discovery and replication cohorts identified 141 SNPs that exceed the threshold for genome-wide significance (p<5 10⁻⁷), implicating 10 regions within the genome. Some of these SNPs have been identified in a GWAS for another autoimmune disease (http://www.genome.gov/gwastudies/): type I diabetes (T1D),^{S10,S11} rheumatoid arthritis (RA),^{S3,S11,S14} systemic lupus erythematosus (SLE),^{S15,S16} multiple sclerosis (MS)^{S11}, celiac disease (CeD),^{S17} or primary biliary cirrhosis (PBC).^{S18} SNPs that were used to obtain the Genetic Liability Index (GLI) are marked with an asterisk. An additional 163 SNPs with nominal significance (1×10⁻⁴>p>5×10⁻⁷) implicate additional immune-related genes. Genes are classified as immune-related either because they were reported as associated with an autoimmune disease (http://hugenavigator.net/) or have been annotated as immune or inflammatory by the Gene Ontology project (http://www.geneontology.org/).

Imputation allowed us to infer genotypes for an additional 2,088,685 SNPs, of which 835 exceed significance of 5×10⁻⁷. Of these, 661 fall within the HLA region. Table 13 lists the 174 significant imputed SNPs that are not in the HLA. Population attributable risk is calculated for independent risk loci (Table 5). Previous to our GWAS, several reports of candidate gene studies have presented evidence for associations in HLA-residing genes (HLA-DQB1, HLA-DRB1, HLA-A, HLA-B, HLA-C, NOTCH4, MICA), as well as genes outside of the HLA (PTPN22, AIRE).^{P24} We compared these findings to results from our GWAS and found that associations to HLA DRB1, HLA-DQB1, HLA-DQA1, and MICA were confirmed (Table 6).

TABLE 13
Statistically significant (p < 5 × 10−7) results
for imputed SNPs in regions outside of the HLA.

| | | position | | | | | | | |
|---|---|---|---|---|---|---|---|---|---|
| chr | SNP | (bp) | alleles | A1 | FREQ1 | OR (L95, U95) | | pvalue | RSQR |
| 2 | rs3116513 | 204402856 | A < G | A | 0.42 | 1.46 | (1.32, 1.61) | 2.5E−13 | 0.971 |
| 2 | rs12992492 | 204409799 | A < G | G | 0.59 | 1.46 | (1.32, 1.61) | 3.6E−13 | 0.986 |
| 2 | rs231775 | 204440959 | A < G | G | 0.62 | 1.44 | (1.3, 1.59) | 3.2E−12 | 0.974 |
| 2 | rs231779 | 204442732 | C < T | T | 0.62 | 1.44 | (1.3, 1.59) | 3.2E−12 | 0.977 |
| 2 | rs11571315 | 204439146 | C < T | T | 0.61 | 1.43 | (1.29, 1.58) | 4.3E−12 | 0.964 |
| 2 | rs3087243 | 204447164 | A < G | A | 0.42 | 0.7 | (0.63, 0.77) | 6.0E−12 | 0.949 |
| 2 | rs736611 | 204438710 | C < T | C | 0.40 | 1.42 | (1.28, 1.57) | 1.1E−11 | 0.966 |
| 2 | rs11571292 | 204428384 | A < G | A | 0.41 | 1.41 | (1.28, 1.57) | 1.7E−11 | 0.995 |
| 2 | rs231770 | 204437398 | C < T | T | 0.60 | 1.41 | (1.28, 1.56) | 1.9E−11 | 0.981 |
| 2 | rs1427680 | 204438040 | A < G | G | 0.60 | 1.41 | (1.28, 1.56) | 1.9E−11 | 0.971 |
| 2 | rs231746 | 204398600 | C < G | G | 0.51 | 0.71 | (0.64, 0.78) | 1.9E−11 | 0.910 |
| 2 | rs11571316 | 204439334 | A < G | A | 0.42 | 0.7 | (0.63, 0.78) | 2.8E−11 | 0.945 |
| 2 | rs960792 | 204457495 | C < T | C | 0.47 | 0.71 | (0.64, 0.79) | 2.9E−11 | 0.992 |
| 2 | rs7600322 | 204462598 | C < T | C | 0.47 | 0.71 | (0.64, 0.79) | 2.9E−11 | 0.996 |
| 2 | rs6748358 | 204465150 | A < C | A | 0.47 | 0.71 | (0.64, 0.79) | 2.9E−11 | 0.996 |
| 2 | rs1427678 | 204466603 | A < G | A | 0.47 | 0.71 | (0.64, 0.79) | 2.9E−11 | 0.996 |
| 2 | rs17268364 | 204486063 | A < G | A | 0.47 | 0.71 | (0.64, 0.79) | 2.9E−11 | 0.988 |
| 2 | rs11571293 | 204425958 | G < T | T | 0.61 | 0.7 | (0.63, 0.78) | 4.5E−11 | 0.986 |
| 2 | rs231811 | 204422136 | G < T | G | 0.40 | 0.7 | (0.63, 0.78) | 4.6E−11 | 0.985 |
| 2 | rs11571291 | 204429377 | C < T | C | 0.40 | 0.7 | (0.63, 0.78) | 6.0E−11 | 0.994 |
| 2 | rs1024162 | 204430404 | A < T | T | 0.60 | 0.7 | (0.63, 0.78) | 6.0E−11 | 0.994 |
| 2 | rs6745050 | 204399783 | C < T | T | 0.59 | 0.71 | (0.64, 0.79) | 1.2E−10 | 0.960 |
| 2 | rs1968351 | 204401981 | A < C | C | 0.59 | 0.71 | (0.64, 0.79) | 1.2E−10 | 0.979 |
| 2 | rs13030124 | 204402508 | A < G | A | 0.40 | 0.71 | (0.64, 0.79) | 1.2E−10 | 0.997 |
| 2 | rs11571304 | 204417021 | A < T | A | 0.40 | 0.71 | (0.64, 0.79) | 2.2E−10 | 0.996 |
| 2 | rs231806 | 204417594 | C < G | C | 0.40 | 0.71 | (0.64, 0.79) | 2.2E−10 | 0.992 |
| 2 | rs863603 | 204403219 | C < T | C | 0.46 | 0.72 | (0.65, 0.8) | 2.4E−10 | 0.998 |
| 2 | rs231734 | 204402525 | A < G | G | 0.54 | 0.72 | (0.65, 0.8) | 2.7E−10 | 0.999 |
| 2 | rs231733 | 204402710 | A < G | A | 0.46 | 0.72 | (0.65, 0.8) | 2.7E−10 | 0.999 |
| 2 | rs6715389 | 204402866 | C < T | C | 0.46 | 0.72 | (0.65, 0.8) | 2.7E−10 | 0.998 |
| 2 | rs3115969 | 204403050 | C < T | T | 0.54 | 0.72 | (0.65, 0.8) | 2.7E−10 | 0.998 |
| 2 | rs231810 | 204420388 | A < G | A | 0.46 | 0.72 | (0.65, 0.8) | 3.5E−10 | 0.962 |
| 2 | rs10490516 | 204404033 | C < T | C | 0.46 | 0.72 | (0.65, 0.8) | 3.5E−10 | 0.998 |
| 2 | rs231790 | 204408819 | G < T | G | 0.46 | 0.72 | (0.65, 0.8) | 3.9E−10 | 0.998 |
| 2 | rs231789 | 204408197 | C < T | C | 0.46 | 0.72 | (0.65, 0.8) | 4.9E−10 | 0.998 |
| 2 | rs231797 | 204414352 | A < G | A | 0.46 | 0.73 | (0.66, 0.8) | 5.5E−10 | 0.998 |
| 2 | rs231799 | 204415662 | C < T | C | 0.46 | 0.73 | (0.66, 0.8) | 5.5E−10 | 0.998 |
| 2 | rs231800 | 204415830 | C < G | G | 0.54 | 0.73 | (0.66, 0.8) | 5.5E−10 | 0.998 |
| 2 | rs231725 | 204448920 | A < G | A | 0.33 | 1.37 | (1.24, 1.52) | 2.9E−09 | 0.991 |
| 2 | rs1427676 | 204449411 | C < T | C | 0.33 | 1.37 | (1.24, 1.52) | 2.9E−09 | 0.993 |
| 2 | rs231727 | 204449795 | A < G | A | 0.33 | 1.37 | (1.24, 1.52) | 2.9E−09 | 0.992 |
| 2 | rs1365965 | 204460115 | C < T | C | 0.33 | 1.35 | (1.22, 1.5) | 1.6E−08 | 0.994 |
| 2 | rs2352546 | 204466991 | A < G | G | 0.67 | 1.35 | (1.22, 1.5) | 1.6E−08 | 0.998 |
| 2 | rs3096852 | 204472663 | C < T | C | 0.33 | 1.35 | (1.22, 1.5) | 1.6E−08 | 1.000 |
| 2 | rs3116523 | 204473059 | G < T | T | 0.67 | 1.35 | (1.22, 1.5) | 1.6E−08 | 1.000 |
| 2 | rs7596727 | 204491827 | C < T | T | 0.51 | 1.33 | (1.2, 1.47) | 2.4E−08 | 0.986 |
| 2 | rs13029135 | 204492457 | A < C | C | 0.51 | 1.33 | (1.2, 1.47) | 2.6E−08 | 0.986 |
| 2 | rs10932027 | 204494719 | A < G | G | 0.51 | 1.33 | (1.2, 1.47) | 2.8E−08 | 0.986 |
| 2 | rs2033171 | 204496401 | C < T | T | 0.51 | 1.33 | (1.2, 1.47) | 2.8E−08 | 0.986 |
| 2 | rs3116521 | 204489086 | C < G | G | 0.51 | 1.33 | (1.2, 1.47) | 3.2E−08 | 0.986 |
| 2 | rs1896493 | 204500654 | A < G | G | 0.51 | 1.33 | (1.2, 1.46) | 3.2E−08 | 0.986 |
| 2 | rs1978594 | 204499714 | G < T | G | 0.49 | 1.32 | (1.2, 1.46) | 4.2E−08 | 0.984 |
| 2 | rs1978595 | 204499774 | C < T | C | 0.49 | 1.32 | (1.2, 1.46) | 4.5E−08 | 0.984 |
| 2 | rs3116505 | 204487426 | C < T | T | 0.67 | 1.34 | (1.2, 1.48) | 5.0E−08 | 0.992 |
| 2 | rs11571310 | 204501543 | C < T | C | 0.49 | 1.32 | (1.19, 1.46) | 5.2E−08 | 0.985 |
| 2 | rs2352551 | 204503002 | C < T | T | 0.51 | 1.32 | (1.19, 1.46) | 5.2E−08 | 0.986 |
| 2 | rs11571309 | 204501584 | G < T | G | 0.49 | 1.32 | (1.19, 1.46) | 5.6E−08 | 0.985 |
| 2 | rs3096863 | 204500977 | C < G | C | 0.33 | 1.33 | (1.2, 1.48) | 6.2E−08 | 0.994 |
| 2 | rs3096859 | 204490820 | C < T | C | 0.33 | 1.33 | (1.2, 1.48) | 7.2E−08 | 0.992 |
| 4 | rs7656035 | 123739679 | A < C | C | 0.65 | 1.35 | (1.22, 1.5) | 6.9E−09 | 1.000 |
| 4 | rs7682481 | 123743476 | C < G | C | 0.35 | 1.35 | (1.22, 1.5) | 6.9E−09 | 0.998 |
| 4 | rs2390351 | 123776174 | C < T | C | 0.35 | 1.35 | (1.21, 1.49) | 1.4E−08 | 0.983 |
| 4 | rs1949946 | 123219411 | C < G | G | 0.51 | 1.33 | (1.21, 1.47) | 1.5E−08 | 0.999 |
| 4 | rs17391154 | 123775643 | A < C | A | 0.35 | 1.33 | (1.2, 1.48) | 4.1E−08 | 0.988 |
| 4 | rs6853169 | 123537515 | A < T | T | 0.61 | 1.31 | (1.19, 1.45) | 1.1E−07 | 0.993 |
| 4 | rs6849146 | 123545541 | C < T | C | 0.39 | 1.31 | (1.19, 1.45) | 1.1E−07 | 0.994 |
| 4 | rs6827839 | 123558465 | A < G | A | 0.39 | 1.31 | (1.19, 1.45) | 1.1E−07 | 0.998 |
| 4 | rs1383043 | 123562066 | A < G | A | 0.39 | 1.31 | (1.19, 1.45) | 1.1E−07 | 0.999 |
| 4 | rs10212828 | 123719561 | C < T | C | 0.38 | 1.31 | (1.19, 1.45) | 1.2E−07 | 0.949 |
| 4 | rs4267747 | 123702512 | A < G | G | 0.61 | 1.31 | (1.19, 1.45) | 1.2E−07 | 0.982 |
| 4 | rs17644013 | 123269087 | A < G | G | 0.61 | 1.31 | (1.18, 1.45) | 1.6E−07 | 0.973 |
| 4 | rs7667439 | 123613261 | G < T | T | 0.61 | 1.3 | (1.18, 1.44) | 2.1E−07 | 0.997 |
| 4 | rs10032704 | 123525673 | C < T | C | 0.39 | 1.3 | (1.18, 1.44) | 2.5E−07 | 0.993 |
| 4 | rs2127511 | 123532038 | C < T | C | 0.39 | 1.3 | (1.18, 1.44) | 2.5E−07 | 0.993 |
| 4 | rs6832214 | 123300910 | C < G | G | 0.61 | 1.3 | (1.18, 1.44) | 3.0E−07 | 0.999 |
| 4 | rs4833817 | 123391694 | G < T | G | 0.39 | 1.3 | (1.17, 1.44) | 3.3E−07 | 0.999 |
| 4 | rs7673567 | 123625434 | C < T | C | 0.38 | 1.3 | (1.17, 1.43) | 3.8E−07 | 0.959 |
| 4 | rs7682281 | 123315936 | C < T | C | 0.39 | 1.29 | (1.17, 1.43) | 4.5E−07 | 0.998 |
| 6 | rs3860823 | 150398219 | C < T | C | 0.41 | 0.61 | (0.55, 0.68) | 2.3E−20 | 1.000 |
| 6 | rs12181819 | 150396358 | A < G | A | 0.41 | 0.61 | (0.55, 0.68) | 7.9E−20 | 1.000 |
| 6 | rs11155696 | 150398976 | A < G | A | 0.41 | 0.61 | (0.55, 0.68) | 7.9E−20 | 1.000 |
| 6 | rs9479481 | 150399637 | A < G | G | 0.59 | 0.61 | (0.55, 0.68) | 7.9E−20 | 1.000 |
| 6 | rs11754987 | 150392897 | A < G | A | 0.41 | 0.61 | (0.55, 0.68) | 8.6E−20 | 0.987 |
| 6 | rs13209192 | 150391792 | A < G | G | 0.59 | 0.61 | (0.55, 0.68) | 9.7E−20 | 0.978 |
| 6 | rs13198863 | 150392474 | G < T | G | 0.41 | 0.61 | (0.55, 0.68) | 1.0E−19 | 0.983 |
| 6 | rs11757186 | 150386067 | A < G | A | 0.41 | 0.62 | (0.56, 0.69) | 4.6E−19 | 0.941 |
| 6 | rs13218129 | 150383233 | C < T | T | 0.58 | 0.62 | (0.56, 0.69) | 1.0E−18 | 0.930 |
| 6 | rs9478362 | 150382219 | C < T | C | 0.41 | 0.63 | (0.57, 0.7) | 3.0E−18 | 0.925 |
| 6 | rs5017316 | 150375182 | A < T | T | 0.59 | 0.63 | (0.57, 0.7) | 3.5E−18 | 0.907 |
| 6 | rs9479405 | 150379758 | A < G | A | 0.41 | 0.63 | (0.57, 0.7) | 3.7E−18 | 0.917 |
| 6 | rs9479403 | 150379439 | C < T | C | 0.41 | 0.63 | (0.57, 0.7) | 4.4E−18 | 0.912 |
| 6 | rs9478354 | 150376059 | A < G | A | 0.41 | 0.63 | (0.57, 0.7) | 4.6E−18 | 0.908 |
| 6 | rs563278 | 150406120 | C < G | C | 0.41 | 0.63 | (0.57, 0.7) | 7.3E−18 | 0.990 |
| 6 | rs9479513 | 150409013 | G < T | T | 0.59 | 0.63 | (0.57, 0.7) | 7.3E−18 | 0.991 |
| 6 | rs2065713 | 150423333 | A < G | A | 0.41 | 1.56 | (1.41, 1.72) | 1.2E−17 | 0.984 |
| 6 | rs932744 | 150432356 | C < G | C | 0.42 | 1.54 | (1.39, 1.71) | 3.9E−17 | 0.985 |
| 6 | rs562425 | 150400892 | A < G | A | 0.44 | 0.64 | (0.58, 0.71) | 4.3E−17 | 0.778 |
| 6 | rs9371693 | 150431128 | A < G | A | 0.42 | 1.52 | (1.37, 1.68) | 5.5E−16 | 0.982 |
| 6 | rs550193 | 150435583 | C < T | T | 0.63 | 1.47 | (1.33, 1.63) | 8.7E−14 | 0.964 |
| 6 | rs912558 | 150427423 | G < T | G | 0.35 | 0.67 | (0.6, 0.75) | 7.1E−13 | 0.987 |
| 6 | rs9397137 | 150423695 | A < G | A | 0.34 | 0.68 | (0.61, 0.75) | 1.1E−12 | 0.982 |
| 6 | rs6941524 | 150423360 | G < T | G | 0.48 | 0.7 | (0.63, 0.77) | 1.9E−12 | 0.985 |
| 6 | rs4869816 | 150436219 | C < G | C | 0.38 | 1.42 | (1.28, 1.57) | 1.4E−11 | 0.964 |
| 6 | rs12202684 | 150420144 | C < T | C | 0.29 | 1.4 | (1.26, 1.56) | 3.8E−10 | 0.981 |
| 6 | rs11756904 | 150452593 | C < T | C | 0.34 | 0.71 | (0.64, 0.79) | 6.8E−10 | 0.995 |
| 6 | rs11754434 | 150452678 | C < T | T | 0.66 | 0.71 | (0.64, 0.79) | 6.8E−10 | 0.997 |
| 6 | rs11756945 | 150452795 | C < T | C | 0.34 | 0.71 | (0.64, 0.79) | 6.8E−10 | 0.998 |
| 6 | rs13216978 | 150453260 | C < T | T | 0.66 | 0.71 | (0.64, 0.79) | 6.8E−10 | 0.945 |
| 6 | rs789825 | 150450639 | A < G | A | 0.34 | 0.71 | (0.64, 0.79) | 7.6E−10 | 0.993 |
| 6 | rs11755079 | 150453451 | A < G | A | 0.35 | 0.71 | (0.64, 0.8) | 8.6E−10 | 0.898 |
| 6 | rs12192777 | 150413828 | C < T | T | 0.70 | 1.39 | (1.25, 1.54) | 1.1E−09 | 0.968 |
| 6 | rs11155698 | 150408547 | C < T | C | 0.26 | 1.39 | (1.25, 1.55) | 1.9E−09 | 0.914 |
| 6 | rs9384068 | 150402575 | A < G | A | 0.28 | 1.38 | (1.24, 1.53) | 4.9E−09 | 0.978 |
| 6 | rs11155699 | 150409590 | C < T | C | 0.28 | 1.38 | (1.24, 1.53) | 4.9E−09 | 0.995 |
| 6 | rs12213731 | 150410455 | A < C | A | 0.28 | 1.37 | (1.23, 1.53) | 6.7E−09 | 1.000 |
| 6 | rs789824 | 150450765 | A < C | A | 0.35 | 0.73 | (0.66, 0.81) | 7.0E−09 | 0.967 |
| 6 | rs6907188 | 150387730 | A < G | A | 0.45 | 1.33 | (1.2, 1.47) | 2.0E−08 | 0.940 |
| 6 | rs4242284 | 150379521 | A < G | G | 0.55 | 1.33 | (1.2, 1.47) | 2.1E−08 | 0.912 |
| 6 | rs6913561 | 150381728 | A < G | A | 0.45 | 1.33 | (1.2, 1.46) | 2.5E−08 | 0.918 |
| 6 | rs639240 | 150385522 | A < C | C | 0.61 | 1.32 | (1.19, 1.46) | 5.1E−08 | 0.938 |
| 6 | rs17079170 | 150389480 | A < G | A | 0.39 | 1.31 | (1.19, 1.45) | 8.7E−08 | 0.955 |
| 6 | rs9322242 | 150447728 | C < T | C | 0.39 | 0.76 | (0.69, 0.84) | 1.9E−07 | 0.999 |
| 6 | rs7767719 | 150447842 | A < G | G | 0.61 | 0.76 | (0.69, 0.84) | 1.9E−07 | 0.998 |
| 6 | rs9322243 | 150448005 | C < G | C | 0.39 | 0.76 | (0.69, 0.84) | 1.9E−07 | 0.997 |
| 6 | rs10457079 | 150423112 | C < T | T | 0.78 | 1.34 | (1.2, 1.51) | 4.9E−07 | 0.933 |
| 9 | rs1830454 | 101757954 | A < G | A | 0.33 | 1.32 | (1.19, 1.46) | 2.2E−07 | 0.999 |
| 9 | rs7027619 | 101759549 | G < T | T | 0.67 | 1.32 | (1.19, 1.46) | 2.2E−07 | 1.000 |
| 9 | rs10121880 | 101724864 | A < G | G | 0.67 | 1.32 | (1.19, 1.46) | 2.4E−07 | 0.963 |
| 9 | rs4282626 | 101730001 | A < G | G | 0.67 | 1.32 | (1.19, 1.46) | 2.4E−07 | 0.964 |
| 9 | rs9299335 | 101731267 | A < G | G | 0.67 | 1.32 | (1.19, 1.46) | 2.4E−07 | 0.965 |
| 9 | rs10123261 | 101735263 | A < C | A | 0.33 | 1.32 | (1.19, 1.46) | 2.4E−07 | 0.968 |
| 9 | rs10120103 | 101735289 | C < T | C | 0.33 | 1.32 | (1.19, 1.46) | 2.4E−07 | 0.969 |
| 9 | rs4742778 | 101741788 | G < T | T | 0.67 | 1.32 | (1.19, 1.46) | 2.4E−07 | 0.972 |
| 9 | rs10760704 | 101748385 | A < G | G | 0.67 | 1.32 | (1.19, 1.46) | 2.4E−07 | 0.975 |
| 9 | rs7038506 | 101749953 | C < T | T | 0.67 | 1.32 | (1.19, 1.46) | 2.4E−07 | 0.978 |
| 9 | rs10217337 | 101750217 | A < G | G | 0.67 | 1.32 | (1.19, 1.46) | 2.4E−07 | 0.981 |
| 9 | rs10217692 | 101750252 | C < T | C | 0.33 | 1.32 | (1.19, 1.46) | 2.4E−07 | 0.983 |
| 9 | rs1852863 | 101752237 | A < G | A | 0.33 | 1.32 | (1.19, 1.46) | 2.4E−07 | 0.993 |
| 9 | rs1997367 | 101753579 | A < G | A | 0.33 | 1.32 | (1.19, 1.46) | 2.4E−07 | 0.997 |
| 9 | rs10512268 | 101754287 | A < G | G | 0.67 | 1.32 | (1.19, 1.46) | 2.4E−07 | 0.996 |
| 9 | rs7039716 | 101710036 | A < T | T | 0.67 | 1.32 | (1.19, 1.46) | 2.6E−07 | 0.958 |
| 9 | rs4585797 | 101713968 | C < G | G | 0.67 | 1.32 | (1.19, 1.46) | 2.6E−07 | 0.958 |
| 9 | rs2416936 | 101716860 | A < T | T | 0.67 | 1.32 | (1.19, 1.46) | 2.6E−07 | 0.959 |
| 9 | rs4742777 | 101718994 | C < T | C | 0.33 | 1.32 | (1.19, 1.46) | 2.6E−07 | 0.960 |
| 9 | rs2416937 | 101720458 | A < C | C | 0.67 | 1.32 | (1.19, 1.46) | 2.6E−07 | 0.960 |
| 9 | rs4743370 | 101721173 | G < T | T | 0.67 | 1.32 | (1.19, 1.46) | 2.6E−07 | 0.961 |
| 9 | rs2416935 | 101709378 | G < T | T | 0.67 | 1.32 | (1.19, 1.46) | 2.7E−07 | 0.877 |
| 9 | rs9556 | 101772058 | C < T | T | 0.67 | 1.32 | (1.19, 1.46) | 2.8E−07 | 0.984 |
| 9 | rs10760700 | 101721632 | A < G | G | 0.66 | 1.31 | (1.18, 1.46) | 3.1E−07 | 0.932 |
| 10 | rs3134883 | 6140731 | A < G | A | 0.30 | 1.48 | (1.33, 1.65) | 1.1E−12 | 0.998 |
| 10 | rs706778 | 6138955 | C < T | T | 0.59 | 1.38 | (1.25, 1.53) | 4.9E−10 | 0.991 |
| 10 | rs12412095 | 6153529 | A < G | G | 0.66 | 1.35 | (1.22, 1.5) | 1.7E−08 | 0.937 |
| 10 | rs10795791 | 6148346 | A < G | G | 0.58 | 1.3 | (1.18, 1.44) | 3.3E−07 | 1.000 |
| 11 | rs574087 | 63859524 | A < G | G | 0.63 | 0.75 | (0.67, 0.83) | 8.4E−08 | 0.970 |
| 11 | rs499425 | 63862505 | A < G | A | 0.35 | 0.75 | (0.67, 0.83) | 1.6E−07 | 0.980 |
| 11 | rs671976 | 63802605 | A < G | G | 0.51 | 1.3 | (1.18, 1.44) | 1.9E−07 | 0.991 |
| 11 | rs1199046 | 63874702 | C < T | T | 0.65 | 0.76 | (0.68, 0.84) | 3.7E−07 | 0.957 |
| 11 | rs663743 | 63864311 | A < G | A | 0.31 | 0.75 | (0.67, 0.84) | 4.1E−07 | 0.936 |
| 12 | rs877636 | 54766850 | A < G | G | 0.66 | 1.35 | (1.22, 1.49) | 1.3E−08 | 0.940 |
| 12 | rs705702 | 54676903 | A < G | G | 0.66 | 1.34 | (1.21, 1.49) | 1.5E−08 | 0.975 |
| 12 | rs2292239 | 54768447 | G < T | T | 0.66 | 1.34 | (1.21, 1.49) | 1.6E−08 | 0.941 |
| 12 | rs2456973 | 54703195 | A < C | C | 0.65 | 1.34 | (1.21, 1.48) | 1.7E−08 | 0.998 |
| 12 | rs705704 | 54721679 | A < G | A | 0.35 | 1.34 | (1.21, 1.48) | 1.7E−08 | 0.993 |
| 12 | rs772921 | 54689844 | C < T | T | 0.65 | 1.34 | (1.21, 1.48) | 1.8E−08 | 0.998 |
| 12 | rs11171739 | 54756892 | C < T | C | 0.42 | 1.33 | (1.2, 1.47) | 2.7E−08 | 0.942 |
| 12 | rs705698 | 54670954 | C < T | C | 0.34 | 1.33 | (1.2, 1.47) | 5.2E−08 | 0.976 |
| 12 | rs2271194 | 54763961 | A < T | A | 0.42 | 1.32 | (1.19, 1.46) | 5.9E−08 | 0.939 |
| 12 | rs773108 | 54656178 | A < G | G | 0.66 | 1.33 | (1.2, 1.47) | 6.2E−08 | 0.992 |
| 12 | rs773109 | 54660962 | A < G | A | 0.34 | 1.33 | (1.2, 1.47) | 6.5E−08 | 0.986 |
| 12 | rs705699 | 54671071 | A < G | A | 0.42 | 1.3 | (1.18, 1.44) | 1.8E−07 | 0.977 |
| 12 | rs2271189 | 54781258 | A < G | A | 0.42 | 1.31 | (1.18, 1.45) | 2.1E−07 | 0.991 |
| 12 | rs773114 | 54665327 | A < T | T | 0.58 | 1.3 | (1.17, 1.43) | 3.4E−07 | 0.979 |
| 12 | rs1873914 | 54665694 | C < G | C | 0.42 | 1.29 | (1.17, 1.43) | 3.7E−07 | 0.978 |
| 18 | rs888270 | 12764894 | A < G | A | 0.18 | 1.39 | (1.22, 1.57) | 3.4E−07 | 0.842 |

Reducing Redundancy in Association Evidence.

When several SNPs that are clustered together within the genome are all significantly associated with a trait, such as is depicted in FIG. 5A, there are two alternative explanations. First, linkage disequilibrium (LD) between the alleles accounts for the association of each SNP with the trait (FIG. 5B). In such a scenario, SNPs reside on a single haplotype which is inherited together, and conditioning on any one of the clustered SNPs will remove evidence of association for the other SNPs, so that the effect estimate of SNP₂ conditioned on SNP, will show no association (OR=1). (FIG. 5B). Alternatively, the effects of the SNPs may be independent, residing on distinct haplotypes which are inherited independently. In this case, conditioning on one SNP will not change the effect estimate of the other SNPs (FIG. 5C). In traditional risk factor epidemiology, these two models are distinguished by confounding analysis. Specifically, either stratified analysis or conditional regression is employed to determine if conditioning on one exposure variable reduces the magnitude of the effect estimate for the second exposure variable.

For the analysis, SAS was used to perform logisitic regression to obtain crude effect estimates for each of the significantly associated SNPs within a given genomic region. For each SNP, we compared this estimate to an adjusted estimate, obtained by entering a second SNP as a covariate. For all regions outside of the HLA, either adjustment did not alter the crude estimate and the SNPs were inferred to be on distinct haplotypes, or adjustment resulted in a null effect estimate (OR=1) and we inferred that the SNPs reside on a common haplotype. Within the HLA, adjustment sometimes altered the effect estimate, though not to the null value. Therefore for analysis of the HLA region, a 10% threshold was used. If the adjusted effect estimate differed from the crude estimate by more than 10%, we concluded the presence of shared haplotypes. The results of these analyses are summarized in Table 3 by an indication of risk haplotype.

Protein and mRNA Distribution of Hair Follicle Related Genes.

Genes that showed statistically significant evidence for association with AA were assessed for expression in the hair follicle and immune system. To determine expression in immune tissues, whole blood cell was subject to PCR. Primers used are listed in Table 9.

Integrating GWAS Results with Previous Genetic Studies in AA.

Prior to this GWAS, we had performed linkage analysis in a cohort of 28 AA families.^{S19} Our GWAS evidence overlaps with linkage at the loci on 6p, 6q and 10p. A comparison of our GWAS results to the previously published linkage studies in the C3H-HeJ mouse model for alopecia areata revealed overlap only within the HLA Class II region.^{S20}

We did not find statistically significant evidence for some of the other candidate genes previously reported for AA, such as AIRE or PTPN22. In Table 6, we summarize published candidate gene studies in AA (obtained from the Human Genetic Epidemiology Navigator; www.HuGEnavigator.net) and compare findings in this study.

Table 6 shows the investigated gene, study conclusion, the number of published studies, and the minimum p-value obtained in our GWAS. Outside of the HLA, none of the genes exceeded the significance threshold in our study, although some may reach significance as our sample size is increased or the GWAS is replicated in other populations.

Peroxiredoxin (PRDX) Gene Family in Autoimmunity.

The mitochondrial respiration and general metabolic activity of cells constantly produce reactive oxygen species which can further oxidize the organelle membranes, proteins or DNA and render them unstable or inactive. There is protective redox enzymatic machinery in cells which reduces these ROS species into harmless byproducts using antioxidants such as glutathione, thioredoxins and others. PRDXs are a family of such enzymes that contain a redox-active cystine residue in their active site which converts H₂O₂ or alkyl peroxides into harmless byproducts^{P25}. Overexpression of PRDX5 protects the cell against DNA damage and apoptosis when subjected to high concentrations of oxidative stress^{P26,P27}.

Chronic upregulation of PRDX5 can ultimately lead to the survival of aberrant cells which harbor danger signals and can present damaged self antigens to the immune system. This can lead to development of autoimmunity. PRDXs themselves can undergo hyperoxidation-induced structural modifications in stressed tissue^{P28}. Autoantibodies against PRDX1, PRDX2, and PRDX4 have observed in a variety of autoimmune disorders^{P29-P31}, as summarized in Table 7.

TABLE 7
Autoimmune diseases with evidence for PRDX autoantigens.

| | Peroxiredoxin Family |
|---|---|
| Disease | Member |
| Systemic sclerosis | PRDX1³⁰ |
| Rheumatoid arthritis | PRDX1, PRDX4³¹ |
| Systemic lupus erythematosus | PRDX1, PRDX4³¹ |
| Psoriasis | PRDX2²⁹ |
| Crohn's disease | AphC (PRDX5)³² |

In Crohn's disease, antibodies were found to AphC (a bacterial homolog of PRDX5)^{P32}. Furthermore, it has recently been demonstrated that PRDX4 is upregulated in synovial tissue of rheumatoid arthritis patients^{P33} and that upregulation is associated with more severe tissue damage in patients with celiac disease^{P34}. It is noteworthy that the mouse homologs of PRDX1 and PRDX2 are located centrally within a region of linkage in the C3H/HeJ mouse model of AA (Alaa3 locus on mouse chromosome 8)^{P35}. PRDX5 levels are elevated in the astrocytes in the multiple sclerosis lesions and in the cartilage tissue in osteoarthritis^{P36,P37}. Interestingly, an alternatively spliced form of PRDX5 has been described which is processed by antigen presentation machinery and can activate the immune system^{P38}.

Aligning the Genetic Architecture of AA with Other Autoimmune Diseases.

CTLA4 plays a role in susceptibility to Graves' disease and Hashimoto's thyroiditis, and interestingly, the frequency of autoimmune thyroid disease has been reported to be significantly higher in AA patients than in healthy controls (25.7% vs. 3.3%; p<0.05).^{S21} In our cohort of AA patients, thyroid disease is found among 16% (Table 8).

TABLE 8
Distribution of autoimmune comorbidities in AA cohort.

| Disease | proband | |
|---|---|---|
| Hay fever/allergic rhinitis | 401 | 37% |
| Allergies | 386 | 35% |
| Atopic Dermatitis/Eczema | 314 | 29% |
| Other Allergies | 275 | 25% |
| Asthma | 205 | 19% |
| Goiter, Graves Disease, Hashimoto's Thyroiditis, | 181 | 17% |
| Hyperthyroidism, Hypothyroidism | | |
| Myxedema; Other Thyroid Disease, Thyroid Disease | | |
| Allergy shots | 144 | 13% |
| Urticaria/Angioedema | 123 | 11% |
| Other Type of Arthritis | 88 | 8% |
| Crohn's disease, Inflammatory bowel disease, | 68 | 6% |
| Irritable bowel syndrome, Ulcerative Colitis | | |
| Arthritis | 58 | 5% |
| Vitilgo | 47 | 4% |
| Psoriasis | 45 | 4% |
| Clinical Depression | 26 | 2% |
| Raynaud's Syndrome | 22 | 2% |
| Diabetes, Insulin Dependent Diabetes Mellitus, | 21 | 2% |
| Non-Insulin Dependent Diabetes Mellitus, Other, Unknown | | |
| Rheumatoid Arthritis | 20 | 2% |
| Fibromyalgia - Fibromyositis | 20 | 2% |
| ADHD | 19 | 2% |
| Hypoparathyroidism | 17 | 2% |
| Glomerulonephritis, IgA nephropathy, Kidney | 14 | 1% |
| Disease Nephrosis, Nephrotic | | |
| syndrome; Other Kidney Disease | | |
| Lichen Planus | 11 | 1% |
| Juvenile Arthritis | 7 | 1% |
| Neurological Disease | 7 | 1% |
| Rheumatic fever | 6 | 1% |
| Autoimmune hemolytic anemia | 6 | 1% |
| Idiopathic thrombocytic purpura | 6 | 1% |
| Systemic Lupus Erythematosus | 5 | 0% |
| Hyperparathyroidism | 5 | 0% |
| Pernicious Anemia | 4 | 0% |
| Cardiomyopathy | 4 | 0% |
| Sjogren's Syndrome | 4 | 0% |
| Collagen vascular disease | 4 | 0% |
| Myasthenia Gravis | 3 | 0% |
| Vasculitis | 3 | 0% |
| Autoimmune Polyendocrinopathy | 3 | 0% |
| Candidiasis-ectodermal dystrophy | | |
| Dermatitis herpetiformis | 3 | 0% |
| Chronic Inflammatory Demyelinating Polyneuropathy | 3 | 0% |
| Bipolar Disease | 2 | 0% |
| Sarcoidosis | 2 | 0% |
| Celiac disease/sprue | 2 | 0% |
| Autoimmune hepatitis | 2 | 0% |
| Uveitis | 2 | 0% |
| Bullous Pemphigoid | 2 | 0% |
| Stiff-man Syndrome | 2 | 0% |
| Autoimmune blistering disease | 2 | 0% |
| Polychondritis | 2 | 0% |
| Multiple Sclerosis | 1 | 0% |
| Polymyalgia Rheumatica | 1 | 0% |
| Spondyloarthritis | 1 | 0% |
| Addison's disease | 1 | 0% |
| CREST Syndrome | 1 | 0% |
| Antiphospholipid Syndrome | 1 | 0% |
| Polymyostis/Dermatomyositis | 1 | 0% |
| Polyarteritis Nodosa | 1 | 0% |
| Sclerodema | | 0% |
| Guillain-Barré syndrome | | 0% |
| Ankylosing spondylitis | | 0% |
| Takayasu Arteritis | | 0% |
| Reiter's Syndrome | | 0% |
| Pemphigus vulgaris | | 0% |
| Churg-Strass syndrome | | 0% |
| Essential Mixed Cryoglobulinemia | | 0% |
| Waardenburg syndrome | | 0% |

In contrast, psoriasis consistently demonstrates strong association to the HLA class I locus, suggesting some fundamental disease mechanisms differ between AA and psoriasis, despite the fact that both affect the skin. Among the most noteworthy correlations include 28% of AA patients also have atopic dermatitis and 16% have thyroiditis, whereas psoriasis and vitiligo are each found in only 4% of our cohort of AA patients (Table 8).

Therapies against several of the genes identified in our GWAS are already in clinical use for some of these disorders. Specifically, CTLA4 blockade by abatacept is used in the treatment of RA, and IL-2R has been targeted using daclizumab in patients with MS.^{S22} Likewise, therapeutics for the other two genes from our GWAS are being developed and have been tested successfully in animals, in particular, an anti-IL-21R fusion protein (IL-21R-Fc) in mouse models of RA and SLE, as well as an anti-NKG2D MAb in the NOD mouse model of T1D in which ULBP ligands are expressed in the pancreatic islets.^{S23} Such modalities may represent viable opportunities for clinical trials in AA patients in the near future.

ULBP mRNA Expression.

The expression of ULBP genes is examined in a variety of cell types, using RNA from normal human keratinocytes (NHKs), human thymus, human scalp, human plucked hair follicle (HF), and freshly dissected dermal papilla (DP). ULBP3 and ULBP4 were strongly expressed in NHKs, thymus, scalp, and HF, whereas ULBP6 was expressed in NHKs, scalp and HF, and ULBP2 and ULBP5 were expressed only in NHKs and thymus.

### REFERENCES

S1. Duvic, M., Norris, D., Christiano, A., Hordinsky, M. & Price, V. Alopecia areata registry: an overview. J Investig Dermatol Symp Proc 8, 219-21 (2003).
S2. Mitchell, M. K., Gregersen, P. K., Johnson, S., Parsons, R. & Vlahov, D. The New York Cancer Project: rationale, organization, design, and baseline characteristics. J Urban Health 81, 301-10 (2004).
S3. Plenge, R. M. et al. TRAF1-C5 as a risk locus for rheumatoid arthritis—a genomewide study. N Engl J Med 357, 1199-209 (2007).
S4. Hunter, D. J. et al. A genome-wide association study identifies alleles in FGFR2 associated with risk of sporadic postmenopausal breast cancer. Nat Genet. 39, 870-4 (2007).
S5. Yeager, M. et al. Genome-wide association study of prostate cancer identifies a second risk locus at 8q24. Nat Genet. 39, 645-9 (2007).
S6. Tworoger, S. S., Eliassen, A. H., Sluss, P. & Hankinson, S. E. A prospective study of plasma prolactin concentrations and risk of premenopausal and postmenopausal breast cancer. J Clin Oncol 25, 1482-8 (2007).
S7. Gunderson, K. L. et al. Whole-genome genotyping. Methods Enzymol 410, 359-76 (2006).
S8. Gunderson, K. L. et al. Whole-genome genotyping of haplotype tag single nucleotide polymorphisms. Pharmacogenomics 7, 641-8 (2006).
S9. Steemers, F. J. et al. Whole-genome genotyping with the single-base extension assay. Nat Methods 3, 31-3 (2006).
S10. Hakonarson, H. et al. A novel susceptibility locus for type I diabetes on Chr12q13 identified by a genome-wide association study. Diabetes 57, 1143-6 (2008).
S11. WTCCC. Genome-wide association study of 14,000 cases of seven common diseases and 3,000 shared controls. Nature 447, 661-678 (2007).
S12. Julia, A. et al. Genome-wide association study of rheumatoid arthritis in the Spanish population: KLF12 as a risk locus for rheumatoid arthritis susceptibility. Arthritis Rheum 58, 2275-86 (2008).
S13. Gregersen, P. K. et al. REL, encoding a member of the NF-kappaB family of transcription factors, is a newly defined risk locus for rheumatoid arthritis. Nat Genet 41, 820-3 (2009).
S14. Steer, S. et al. Genomic DNA pooling for whole-genome association scans in complex disease: empirical demonstration of efficacy in rheumatoid arthritis. Genes Immun 8, 57-68 (2007).
S15. Horn, G. et al. Association of Systemic Lupus Erythematosus with C8orf13-BLK and ITGAM-ITGAX. N Engl J Med (2008).
S16. Harley, J. B. et al. Genome-wide association scan in women with systemic lupus erythematosus identifies susceptibility variants in ITGAM, PXK, KIAA1542 and other loci. Nat Genet. 40, 204-10 (2008).
S17. van Heel, D. A. et al. A genome-wide association study for celiac disease identifies risk variants in the region harboring IL2 and IL21. Nat Genet. 39, 827-9 (2007).
S18. Hirschfield, G. M. et al. Primary biliary cirrhosis associated with HLA, IL12A, and IL12RB2 variants. N Engl J Med 360, 2544-55 (2009).
S19. Martinez-Mir A, Zlotogorski A, Gordon D, et al. Genomewide scan for linkage reveals evidence of several susceptibility loci for alopecia areata. Am J Hum Genet. 2007; 80:316-28.
S20. Sundberg J P, Silva K A, Li R, Cox G A, King L E. Adult-onset Alopecia areata is a complex polygenic trait in the C3H/HeJ mouse model. The Journal of investigative dermatology 2004; 123:294-7.
S21. Kasumagic-Halilovic E. Thyroid autoimmunity in patients with alopecia areata. Acta Dermatovenerol Croat 2008; 16:123-5.
S22. Bielekova B, Howard T, Packer A N, et al. Effect of anti-CD25 antibody daclizumab in the inhibition of inflammation and stabilization of disease progression in multiple sclerosis. Arch Neurol 2009; 66:483-9.
S23. Ettinger R, Kuchen S, Lipsky P E. Interleukin 21 as a target of intervention in autoimmune disease. Ann Rheum Dis 2008; 67 Suppl 3:iii83-6.
P24. Gilhar, A., Paus, R. & Kalish, R. S. Lymphocytes, neuropeptides, and genes involved in alopecia areata. J Clin Invest 117, 2019-27 (2007).
P25. Wood, Z. A., Schroder, E., Robin Harris, J. & Poole, L. B. Structure, mechanism and regulation of peroxiredoxins. Trends Biochem Sci 28, 32-40 (2003).
P26. Banmeyer, I. et al. Overexpression of human peroxiredoxin 5 in subcellular compartments of Chinese hamster ovary cells: effects on cytotoxicity and DNA damage caused by peroxides. Free Radic Biol Med 36, 65-77 (2004).
P27. Zhou, Y. et al. Mouse peroxiredoxin V is a thioredoxin peroxidase that inhibits p53-induced apoptosis. Biochem Biophys Res Commun 268, 921-7 (2000).
P28. Schroder, E., Brennan, J. P. & Eaton, P. Am J Physiol Heart Circ Physiol 295, H425-33 (2008).
P29. Besgen, P., Trommler, P., Vollmer, S. & Prinz, J. C. Ezrin, maspin, peroxiredoxin 2, and heat shock protein 27: potential targets of a streptococcal-induced autoimmune response in psoriasis. J Immunol 184, 5392-402.
P30. Iwata, Y. et al. Rheumatology 46, 790-795 (2007).
P31. Karasawa, R., Ozaki, S., Nishioka, K. & Kato, T. Autoantibodies to peroxiredoxin I and IV in patients with systemic autoimmune diseases. Microbiol. Immunol 49, 57-65 (2005).
P32. Olsen, I., Wiker, H. G., Johnson, E., Langeggen, H. & Reitan, L. J. Elevated antibody responses in patients with Crohn's disease against a 14-kDa secreted protein purified from Mycobacterium avium subsp. paratuberculosis. Scand J Immunol 53, 198-203 (2001).
P33. Chang, X. T. et al. Identification of Proteins with Increased Expression in Rheumatoid Arthritis Synovial Tissues. Journal of Rheumatology 36, 872-880 (2009).
P34. Simula, M. P. et al. PPAR signaling, pathway and cancer-related proteins are involved in celiac disease-associated tissue damage. Mol Med 16, 199-209.
P35. Sundberg, J. P., Silva, K. A., Li, R. H., Cox, G. A. & King, L. E. Adult-onset alopecia areata is a complex polygenic trait in the C3H/HeJ mouse model. Journal of Investigative Dermatology 123, 294-297 (2004).
P36. Holley, J. E., et al., Peroxiredoxin V in multiple sclerosis lesions: predominant expression by astrocytes. Mult Scler 13, 955-61 (2007).
P37. Wang, M. X. et al. Expression and regulation of peroxiredoxin 5 in human osteoarthritis. FEBS Lett 531, 359-62 (2002).
P38. Sensi, M. et al. Peptides with dual binding specificity for HLA-A2 and HLA-E are encoded by alternatively spliced isoforms of the antioxidant enzyme peroxiredoxin 5. Int Immunol 21, 257-68 (2009).

### Example 3

### Expression of ULBP3 in Hair Follicle Dermal Sheath in Active AA Lesions

The distribution of ULBP3 protein was examined within the hair follicle of unaffected scalp (FIG. 4B) and in the hair follicles of AA patients (FIG. 4C). Whereas ULBP is expressed at low levels with the hair follicle dermal papilla in normal hair follicles (FIGS. 4A-B), strikingly, in two different patients with early active AA lesions, marked upregulation of ULBP3 expression was observed in the dermal sheath as well as the dermal papilla (FIGS. 4B-C). A massive inflammatory cell infiltrate within the dermal sheath characterized by CD8+CD3+ T cells (FIGS. 4G-L) was noted, but only rare NK cells. Finally, double-immunostainings with an anti-CD8 and an anti-NKG2D antibodies revealed that most CD8+ T cells co-expressed NKG2D (FIGS. 4M-O). These results suggest that the autoimmune attack in AA region is mediated by CD8+NKG2D+ cytotoxic T cells of which infiltration may be induced by upregulation of the NKG2D ligand ULBP3 in the dermal sheath of the HF.

### Example 4

### Danger Signals in the Hair Follicle

We will test whether the origin of autoimmunity in Alopecia Areata (AA) resides in the hair follicle itself. We will focus on defining putative danger signals in the hair follicle that contribute to the pathogenesis of AA. We have selected two candidate genes identified in our recent GWAS study, implicated eight genomic regions involved in AA. Using a battery of in vivo and in vitro approaches, in both human tissue and mouse models, we will systematically define the role of ULBP3/6 and PRDX5 in the hair follicle. This will provide new insights into both the role of PRDX5 and ULBP3/6 genes in AA pathogenesis, as well as modeling the disease in transgenic animals. We will also identify pathogenic alleles that reside within the MHC, which may contribute to immune dysregulation driving the pathogenesis of AA.

We will perform high resolution HLA typing of the DR and DQ loci. Furthermore, we will use integrative analytic methods to identify putative danger signals emitted by the HF.

AA Susceptibility Genes in the Hair Follicle.

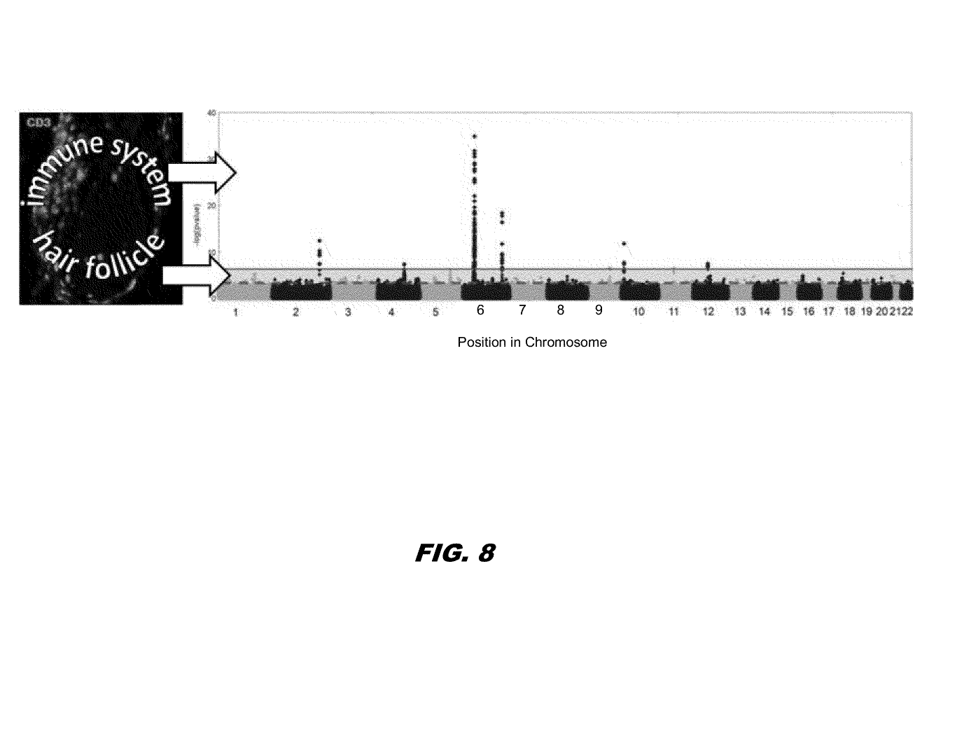

GWAS identify disease alleles that are both associated with disease and exist at sufficient frequencies to be adequately captured by tagSNPs. Immune response genes are vulnerable to positive selection, which increases allele frequencies, thus making this class of genes amenable to detection with GWAS (FIG. 8 upper arrow). While the genetic architecture of AA will be composed of immune genes and hair genes, without being bound by theory, SNPs that exceed statistical significance will largely map to immune genes and hair genes will generally only achieve nominal significance.

In order to mine this ‘gray zone’ of significance (5×10⁻⁷>p>0.01) for hair genes (FIG. 8 lower arrow), we mapped the top 5000 SNPs to a set of 3347 genes. Next, we cross-referenced this gene list with our database of hair follicle genes, which contains 4166 genes that have been implicated in one or more hair follicle gene expression experiments, thus identifying a set of 476 genes. Of these, 5 genes contained SNPs that exceeded statistical significance in the GWAS (p<5×10⁻⁷; PPP1R14C, CREBL1, SUOX, CDK2, STX17). The vast majority of hair genes (471) contained SNPs in the gray zone of significance.

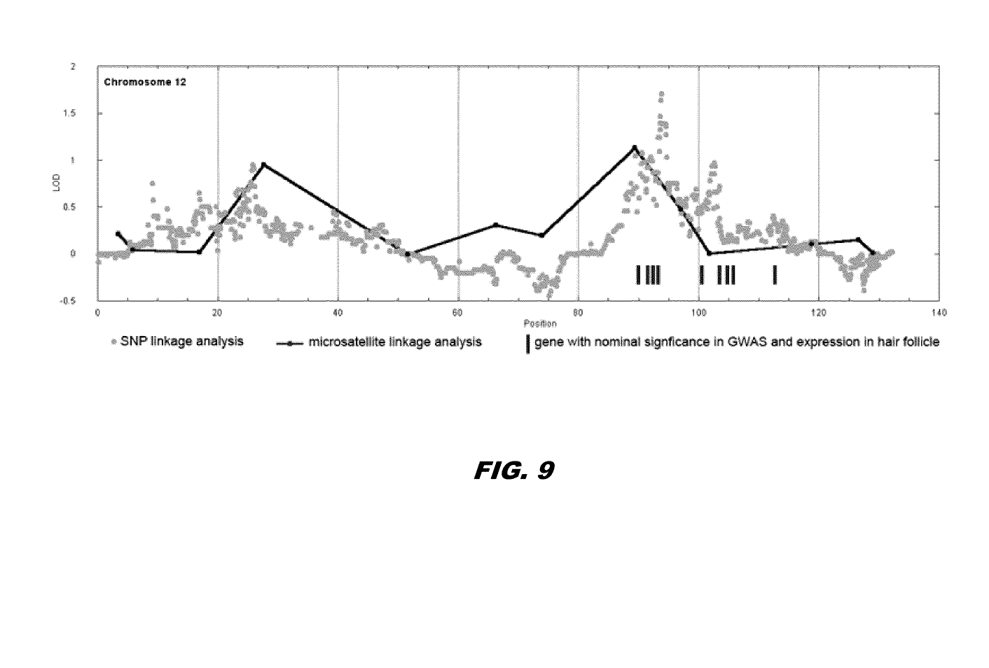

Without being bound by theory, if the distribution of p-values for hair genes are largely driven by low allele frequencies, then results from a method that is suited for detection of rare variants, e.g. linkage, can converge with this “high-hanging fruit” from our GWAS. We therefore cross-referenced the 471 GWAS genes with results from our linkage analyses, and 121 genes fell into regions with at least suggestive evidence for linkage (1<LOD<4). We show results for chromosome 12 (FIG. 9). This indicates that there are biologically relevant hair follicle genes nested within our nominally signficant findings. Next, in order to further characterize these target organ genes, we annotated the list of 476 genes with GO terms and the most significantly represented GO terms related to biological processes involving cell adhesion, motion/locomotion/migration, proliferation and morphogenesis (Table 14).

TABLE 14
Significantly represented GO terms related to biological processes.

| Term | Count (%) | PValue | Genes |
|---|---|---|---|
| cell adhesion | 63 (14%) | 1.14E−14 | CLSTN2, MEGF10, DDR2, SDC3, NRCAM, APP, DAB1, ROBO2, ESAM, COL11A1, |
| (GO: 0007155) | | | PTPRK, PTPRM, PDPN, NRXN3, ACTN1, PTPRU, NRXN1, CD164, CTNNA2, NCAM1, |
| | | | CD36, CNTN1, JAM2, PARVA, PLXNC1, CCR1, TNC, COL3A1, PTK7, CTNND2, |
| | | | SPOCK1, CX3CL1, CDH4, CDH5, ALCAM, CDH8, CD9, ITGB8, COL27A1, PVRL3, |
| | | | BCL2, TEK, SCARB1, THBS1, THBS4, DPT, FLRT3, COL18A1, PTPRC, COL13A1, |
| | | | PCDH10, PCDH17, COL5A1, PCDH18, LAMA2, CDH13, VWF, COL19A1, PKP1, |
| | | | PKP4, PERP, CDH10, CDH11 |
| cell motion | 47 (10%) | 1.94E−12 | CAV2, EDN3, NDN, FUT8, PLXNA2, EDN2, SPOCK1, CX3CL1, TPM1, CDH4, TGFB2, |
| (GO: 0006928) | | | ALCAM, NRCAM, CTTNBP2, CD9, APP, DAB1, DNER, LHX2, PAK4, ROBO2, LHX6, |
| | | | SCARB1, STRBP, SEMA3A, THBS1, RUNX3, DCLK1, THBS4, PTPRK, KLF7, PTPRM, |
| | | | EGR2, NRXN3, ARID5B, OTX2, NR4A2, IGF1, NRXN1, COL5A1, CTNNA2, LSP1, |
| | | | VEGFC, CDH13, EPHA7, ETS1, LRP6 |
| regulation of cell | 61 (13%) | 2.03E−11 | EDN3, E2F3, EDN2, MITF, JAG2, PRRX2, DDR2, TGFB2, CTTNBP2, CASP3, |
| proliferation | | | SERPINE1, PDGFC, ASPH, NRG1, PTPRK, PTPRM, CTBP2, RXRA, CDK6, PTPRU, |
| (GO: 0042127) | | | CD164, CDK2, VEGFC, CTH, HIPK2, VEGFA SCIN, ADAMTS1, SMARCA2, VIP, CAV2, |
| | | | NDN, TAC1, CDH5, MSX2, CD9, BCL2, TEK, CAMK2D, AXIN2, THBS1, RUNX2, |
| | | | RUNX3, DPT, COL18A1, BMP4, PTPRC, BMP2, TBX3, TGFBR2, CD276, SMAD3, |
| | | | IGF1, FOXP1, CDH13, PLA2G4A, NOTCH1, ETS1, SP6, ID4, KLF4 |
| cellular component | 37 (8%) | 3.41E−09 | NDN, PTK7, PIP5K1C, TPM1, CDH4, TGFB2, NRCAM, ALCAM, CD9, APP, DAB2, |
| morphogenesis | | | SLC1A3, LHX2, BCL2, ROBO2, SEMA3A, RUNX3, DCLK1, COL18A1, KLF7, BMP2, |
| (GO: 0032989) | | | PTPRM, EGR2, PDPN, RYK, NRXN3, RXRA MAP1B, OTX2, NR4A2, NRXN1, GAS7, |
| | | | CTNNA2, TNNT2, SS18, EPHA7, NOTCH1 |
| regulation of locomotion | 23 (5%) | 7.52E−08 | COL18A1, PTPRK, EDN3, PLD1, PTPRM, PDPN, EDN2, SNCA, JAG2, SMAD3, TAC1, |
| (GO: 0040012) | | | IGF1, PTPRU, TPM1, TGFB2, LAMA2, CDH13, VEGFC, BCL2, TEK, VEGFA, SCARB1, |
| | | | THBS1 |

We also find 62 genes involved with the regulation of apoptosis or cell death among hair follicle genes with nominal significance in our GWAS. This is noteworthy because the Danger Model of Autoimmunity, which maintains that the primary goal of the immune system is not to distinguish between self and nonself, but rather to distinguish between dangerous and harmless signals, predicts the presence of signals released by cells undergoing abnormal cell death, or normal cell death that has gone awry. Without being bound by theory, such a danger signal is can be an initiating event in autoimmunity.

High Resolution HLA Typing

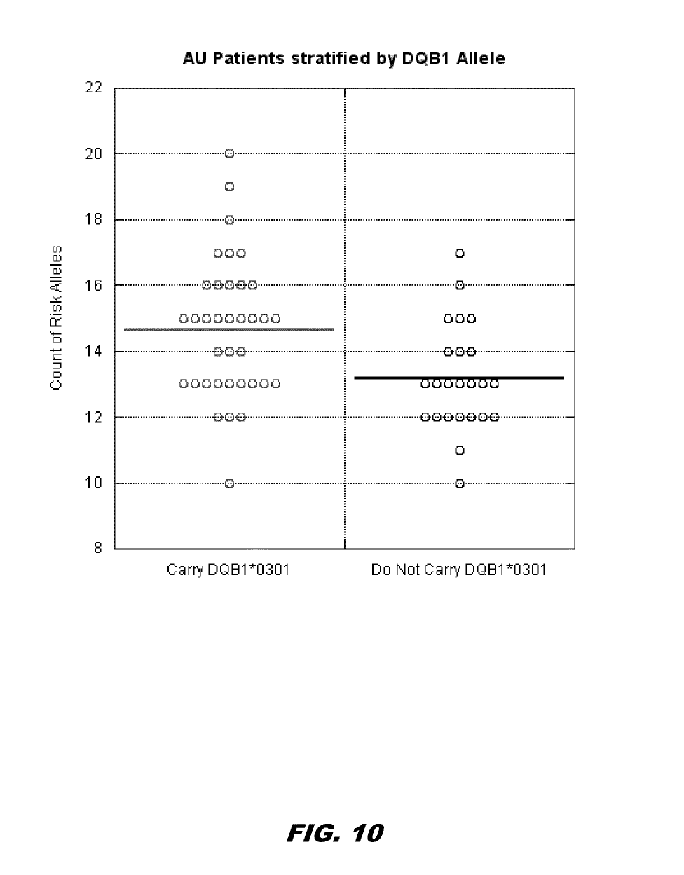

We previously performed high resolution typing (LABType SSO Typing Test from One Lambda, Inc) to genotype a small subset of patients with severe disease (AU) from our GWAS cohort at the DRB1 locus (FIG. 10). We have extended this work by typing this same set of 60 AU patients at the DQB1 and DQA1 locus, allowing us to determine genotypes and serotype groups. HLA class II molecules DQ8 and DQ2 have been identified as key genetic risk factors in T1D and CeD. While DQ8 conveys a higher risk for T1D, DQ2 is more frequent in CD. In our cohort of 60 patients, 43% carried at least one of these risk factors, with 15 patients carrying DQ8 alleles and 13 DQ2. For the HLA-DRB1 locus, allele DRB1*0301 is the only one associated with risk for T1D, CeD and Addison's Disease. In our cohort, this was the most frequent DRB1 allele, present in 36 of our patients (60%). Interestingly, we also observed that patients who carry this risk allele tend to carry a greater genetic liability. In our GWAS, the total number of risk alleles carried by an individual varied significantly between cases and controls. Here, we observe that AU patients who carry DQB1*0301 carry and average of 15 risk alleles across their genomes, while those without this HLA allele, carry an average of 13 risk alleles. Finally, we found four patients that carry the HLA haplotype associated with risk for polymyositis (HLA-DRB1*03-DQA1*05-DQB1*02).

CNVs in AA

We previously scanned the eight regions of statistically significant association from our GWAS in a cohort of unaffected individuals across to catalogue DNA copy number variations (CNVs), and detected variations in STX17, IL2RA and numerous HLA genes. Here, we report our recent results obtained by utilizing a bioinformatic approach that leverages the fact that most common CNVs are well tagged by SNPs found on commercial genotyping arrays. Recently, 3432 polymorphic CNVs have been directlyt yped in a cohort of 19,000 individuals, which had been previously genotyped with commercial SNP arrays. By integrating these two datasets, each CNV was annotated with the best tagSNP from each of several sources (HapMap, Affymetrix, Illumina). We cross-referenced the list of Illumina SNPs with the results of our GWAS and identified three SNPs with evidence for a statistically significant association to AA and correlation to a common CNV (Table 15). We are validating this finding in our cohort of patients.

TABLE 15
AA associated SNPs correlated to a CNV.

| | | AA GWAS | | | StartCoord | EndCoord | |
|---|---|---|---|---|---|---|---|
| CNV | tagSNP | pvalue | CNV allele | Chr | (bp) | (bp) | Size |
| CNVR2843 | rs389884 | 4.97E−07 | CNVR2843.4 | 6 | 32,055,886 | 32,060,381 | 4,495 |
| | | | CNVR2843.2 | 6 | 32,060,426 | 32,066,895 | 6,469 |
| | | | CNVR2843.1 | 6 | 32,093,119 | 32,099,722 | 6,603 |
| | | | CNVR2843.5 | 6 | 32,099,567 | 32,124,504 | 24,937 |
| CNVR2845 | rs1063355 | 2.46E−11 | CNVR2845.27 | 6 | 32,710,664 | 32,743,652 | 32,988 |
| | | | CNVR2845.40 | 6 | 32,735,154 | 32,737,954 | 2,800 |
| CNVR3101 | rs11155699 | 4.92E−09 | CNVR3101.1 | 6 | 150,416,978 | 150,418,278 | 1,300 |

### Example 5

### Regulation of NKG2D Ligands

### The Transcriptional Regulation of NKG2D Ligands by NF-κB

Regulation of ULBP3 and ULBP6 promoters by NF-κB-inducing cytokines and by direct overexpression of NF-κB was shown, and that NF-κB p65 is required for TNF or LPS-induced NKG2DL upregulation in mouse skin was also shown. Comprehensive panels of luciferase constructs driven by 5′ and intronic promoter/enhancer regions of both human and mouse NKG2DL genes are being generated.

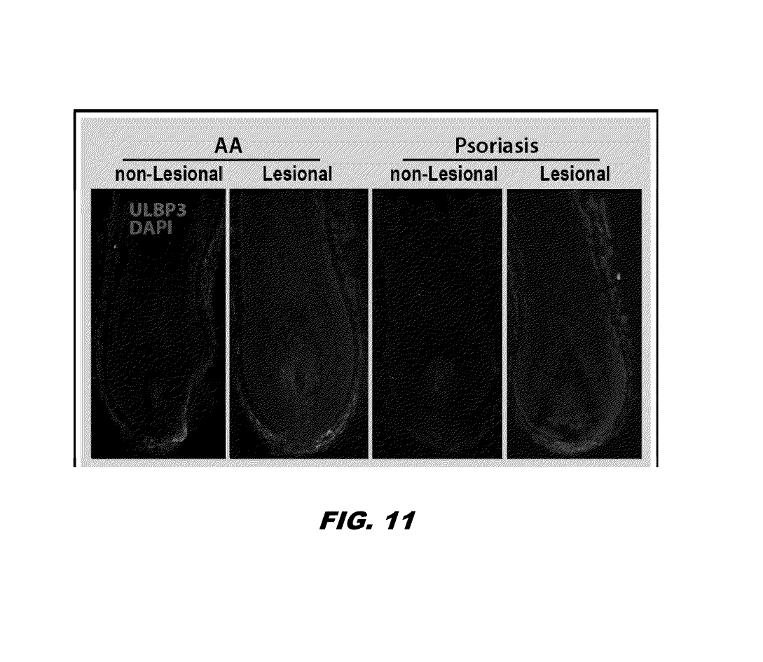

Previously, the analysis of NKG2DL expression in lesional biopsies consistently revealed ULBP3 and MICA upregulation in the alopecic and remission HF. Consistently, H60 and Rae1 are upregulated in the alopecic C3H/HeJ HF. However, it is important to establish that upregulation of NKG2DL at the HF precedes pathology and contributes to the etiology. Therefore, unaffected HFs from AA patients were analyzed. It is found that ULBP3 is increased at unaffected sites (FIG. 11), consistent with the idea that genetic predisposition to AA can be etiologically linked to elevation of NKG2DL that precedes disease onset. Without being bound by theory, access of NKG2D⁺CD8⁺ T cells to HF NKG2DL can be dependent on a second hit. This is completely consistent with recent animal models in which tissue restricted NKG2DL overexpression has been shown to drive antigen-independent NKG2D⁺CD8⁺ T cell responses, leading to tissue damage and induction of adaptive immune responses. It appears these responses are augmented by either tissue injury or the presence of large numbers of NKG2D⁺CD8⁺ T cells.

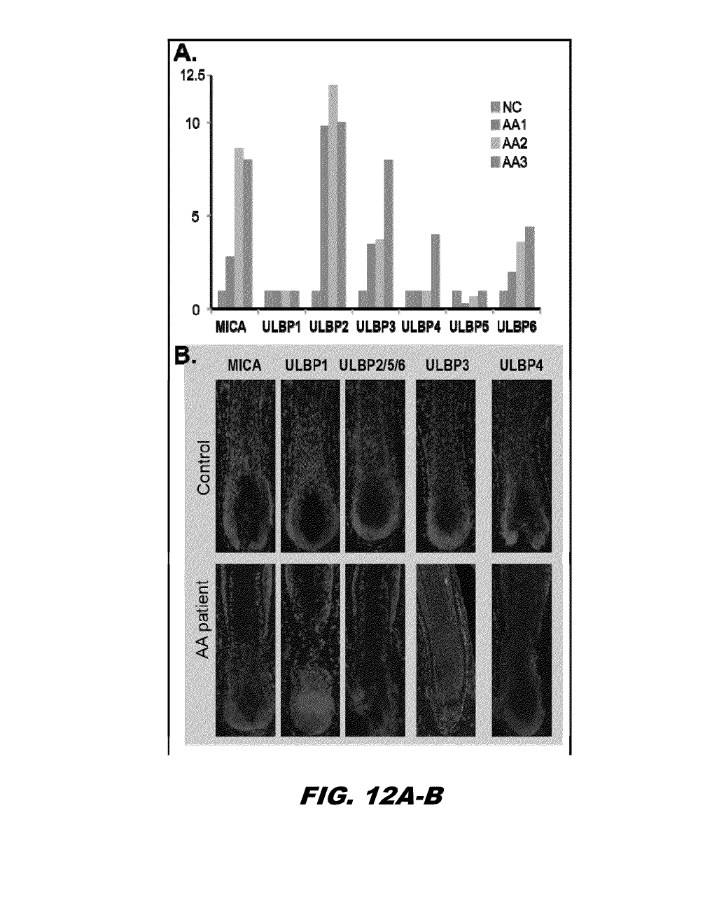

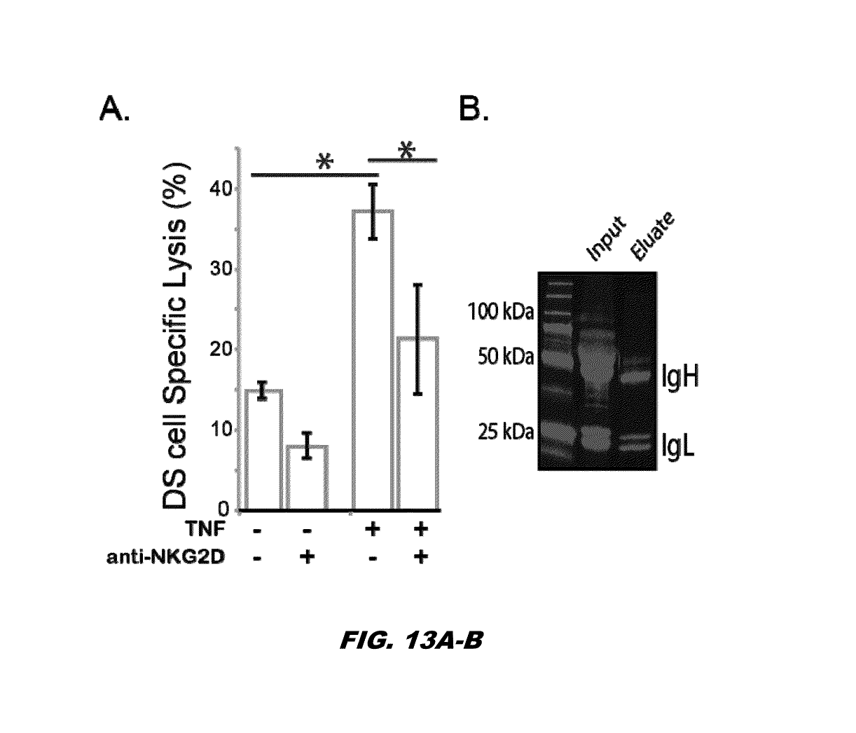

While ULBP3, ULBP6 and MICA were identified in the GWAS study, it is not clear whether others correlate with AA. Therefore, the analysis of ULBP expression (FIG. 12) has been expanded. MICA, ULBP2, ULBP3, and ULBP6 mRNA is increased in AA skin. MICA and ULBP3 proteins are selectively upregulated in the AA HF, while ULBP4 is highly expressed in both control and AA (FIG. 12). Quantitative PCR indicates that the increased ULBP2 expression is responsible for the pan ULBP2/5/6 staining in the AA HF.

### ULBP3 and PRDX5 Expression in Normal and AA HFs

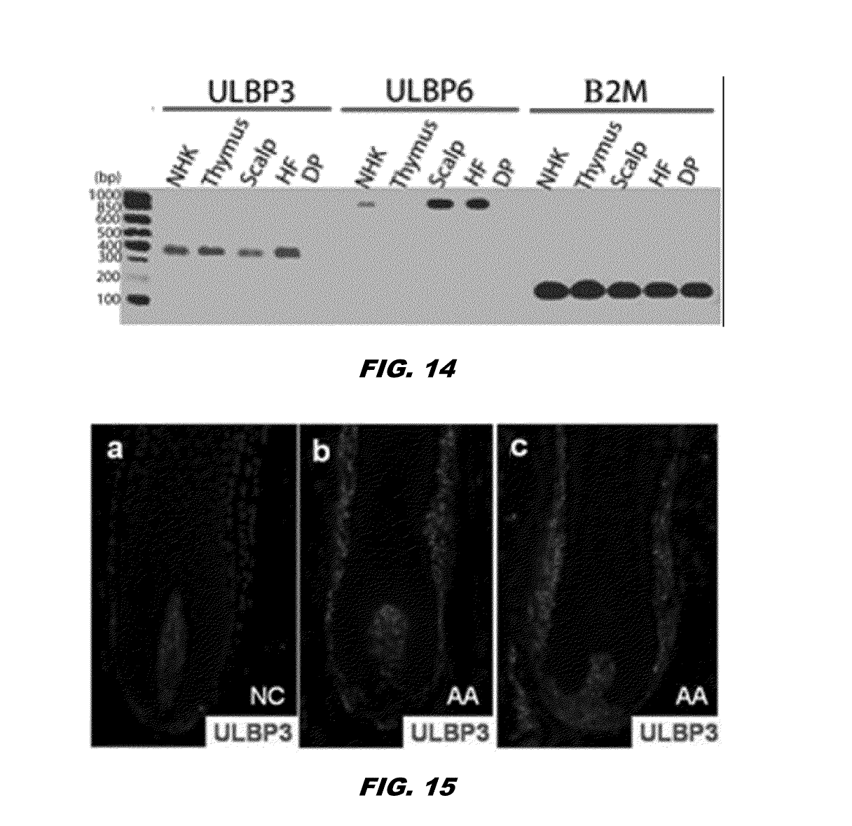

The expression of ULBP genes implicated by the AA GWAS was examined in a variety of cell types. RNA was extracted from normal human keratinocytes (NHKs), human thymus, human scalp, human plucked HF (HF), and freshly dissected dermal papilla (DP). Expression analysis in scalp, HF and DP was performed in order to determine the particular niche of gene expression. NHKs and thymus were used as expression controls and B2M was used as a cDNA loading control. ULBP3 and was strongly expressed in NHKs, thymus, scalp, and HF. ULBP6 was found to be expressed in NHKs, scalp and HF. Neither gene member showed expression in freshly dissected DP cells (FIG. 14). This data shows a variety of expression patterns for the ULBP genes within the tissue affected by AA.

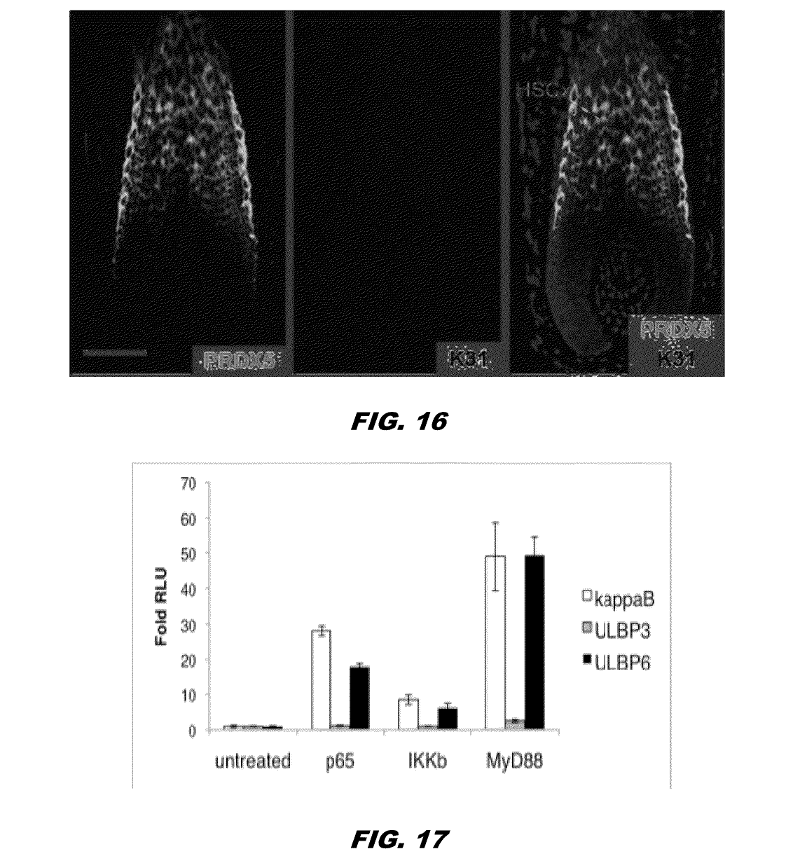

Immunofluorescence was used to localize expression of ULBP3 (FIG. 15) and PRDX5 (FIG. 16) in the HF. In normal scalp, low levels of expression of ULBP3 in the dermal papilla. PRDX5 is expressed in hair shaft and IRS of the human HF, where its expression overlaps with keratin 31 in the hair shaft cortex (HSCx). Right panels are merged images and counterstaining with DAPI is shown in blue (FIGS. 15, 16). Scale bars: 100 μm.

### NKG2D Ligand Expression in Response to Agents of Stress

The surface expression of NKG2DL is regulated at a transcriptional and post translational level. At the transcriptional level, the promoter regions contain stress response elements, as well as different putative transcription factor binding sites that influence tissue specific expression (Eagle et al. 2006). AA is associated with elevated levels of proinflammatory cytokines such as IFNg, TNFa, IL1 and IL-6 (Barahmani et al.; Ghoreishi et al.). A neurogenic stress component is also associated with AA skin with elevated expression of stress hormones such as CRH, Substance P and ACTH. (Kim et al. 2006) (Hordinsky et al. 2004). Higher levels of oxidative stress has also been identified in patients' scalp (Akar et al. 2002). Human dermal sheath (DS) cells, fibroblasts and keratinocytes were cultured in the presence of inflammatory cytokines, stress hormones and oxidative stress inducing conditions, and the transcript levels of ULBP3 and MICA were assessed. The effect of cytokine (IL-13, IL-6, IL-26) identified from the GWAS study will be further identified to determine the role of these cytokines on NKG2DL expression in the skin and the HF.

TABLE 16
Transcript Expression of MICA and ULBP3 under
conditions of stress in skin components.

| | | Hu DS Cells | | Hu fibroblasts | | Hu Keratinocytes | |
|---|---|---|---|---|---|---|---|
| | | ULBP3 | MICA | ULBP3 | MICA | ULBP3 | MICA |
| Genotoxic | UV | 1.4 | 1.1 | 1.45* | 1.96* | 1.58* | 1.2 |
| Stress | H2O2 | 0.55 | 0.95 | 0.96 | 1.25 | 1.01 | 1.27 |
| | 1 mM | | | | | | |
| | Heat | 0.91 | 0.94 | 0.68* | 1.64* | 1.63* | 2.32* |
| | Shock | | | | | | |
| Stress | CRH | 1.56 | 0.96 | 0.87 | 1.07 | 1.18 | 0.35 |
| Hormones | SP | 0.93 | 1.32 | 0.84 | 0.9 | 0.87 | 0.55 |
| | Hydrocortisone | 0.99 | 1.23 | 0.63 | 1.66 | 0.82 | 0.33 |
| Inflammatory | TNFα | 0.935 | 1.03 | 0.66 | 2.3 | 0.39 | 0.46 |
| Cytokines | IFNγ | 1.68 | 1.53 | 0.41 | 2.14 | 0.49 | 0.55 |

NKG2D recognizes MHC family proteins including the ULBP/RAET1 (UL-16 binding protein; Rae1 and H60 in mice) and MICA/MICB families of proteins. Acute upregulation of NKG2D ligands in the skin is sufficient to trigger an inflammatory response and is of particular interest in both autoimmunity and tumor immunity as ligation of NKG2D is sufficient to provide co-stimulatory signals to both conventional α/β TCR and γ/δ TCR T cells. Thus, without being bound by theory, NKG2D ligation can serve to break peripheral tolerance and/or promote adaptive responses to altered self in both physiological immunity and autoimmune disease states. As the current work concerns the etiology of AA, it is of significant interest that NKG2D on epidermal hematopoietic cells can provide a crucial signal during the response to cultured keratinocytes. Furthermore, NK cell activation correlating with upregulation of the NKG2DL, MICA, has been implicated in the breakdown of hair follicle immune privilege (HF-IP) in AA. Given the ability of NKG2D ligands to provide co-stimulatory signals to α/β T cells and to elicit pro-inflammatory and cytolytic responses, there is growing interest in the role of NKG2DL expression in the etiology of autoimmune diseases.

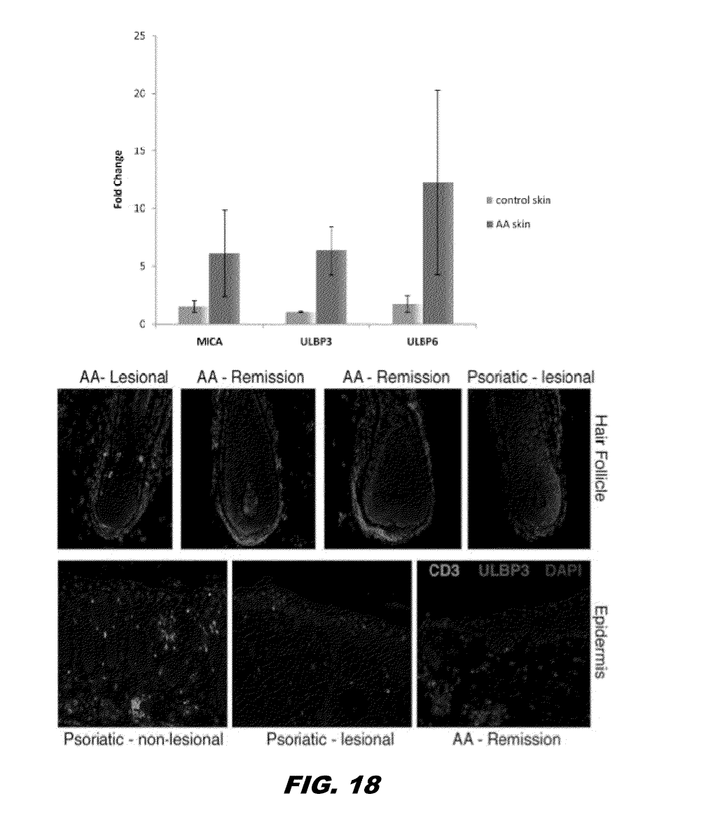

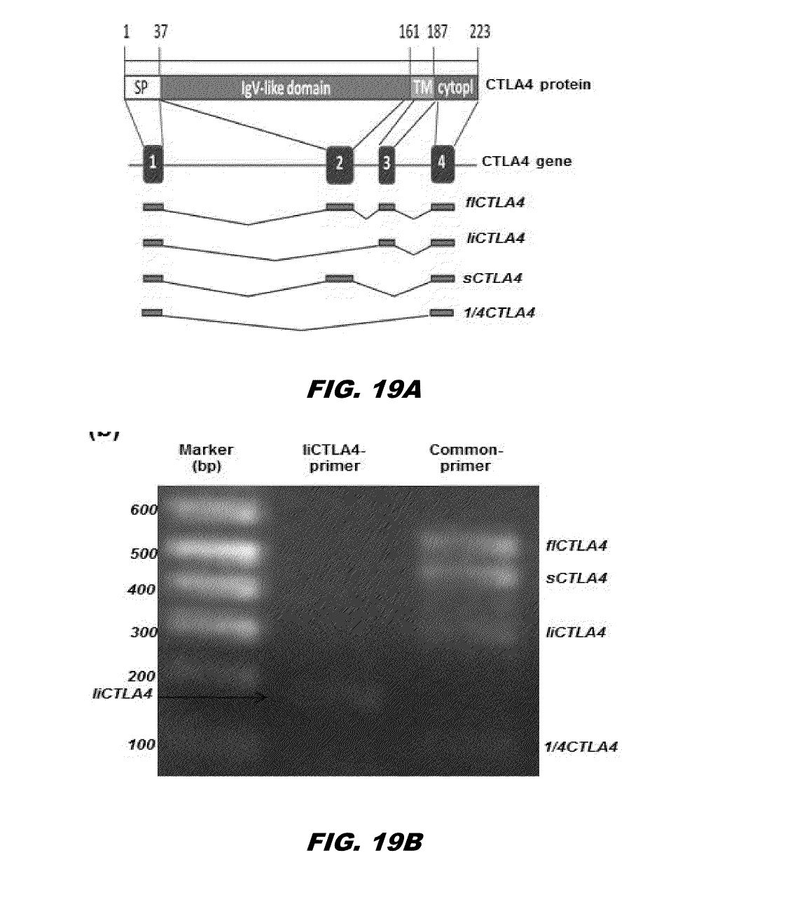

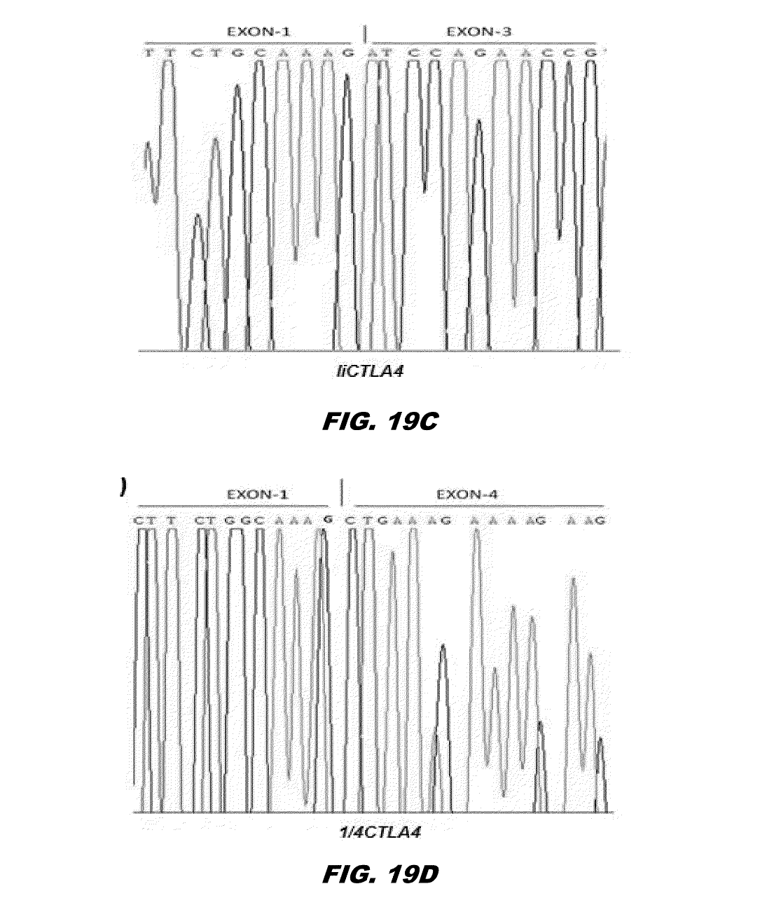

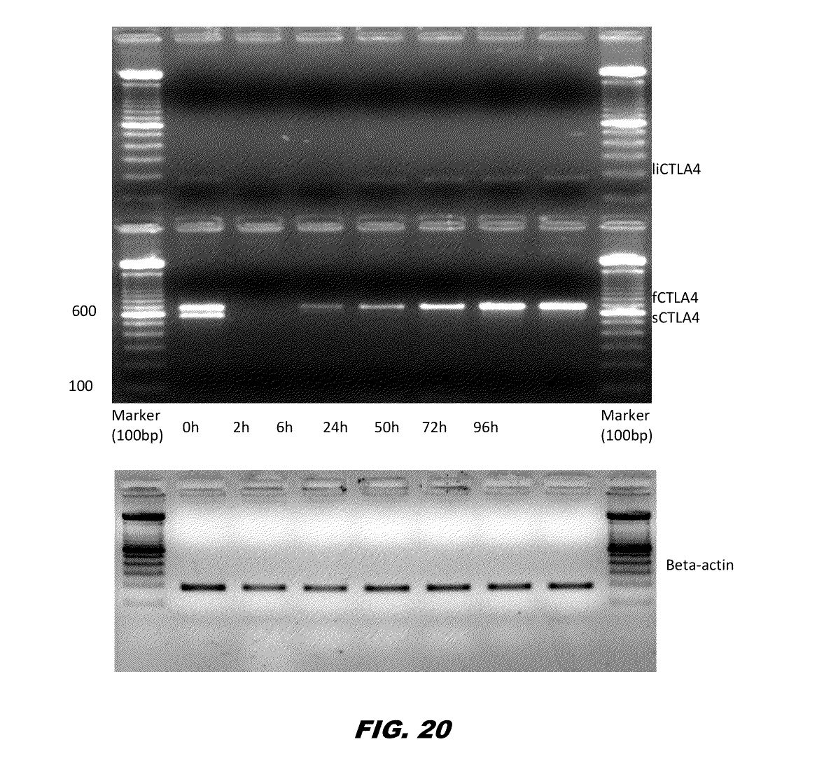

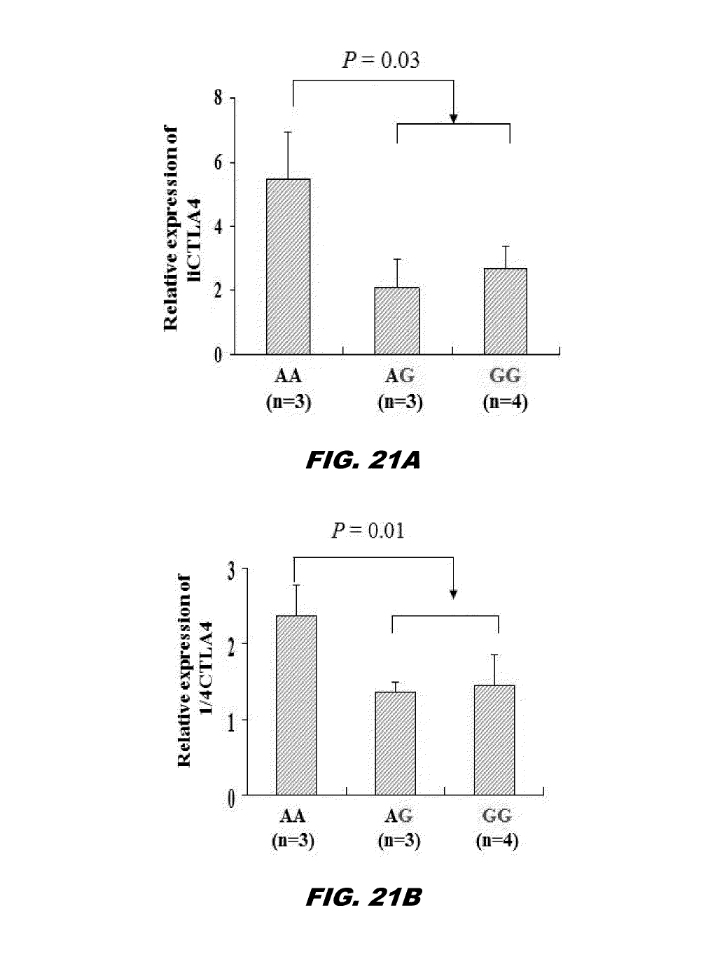

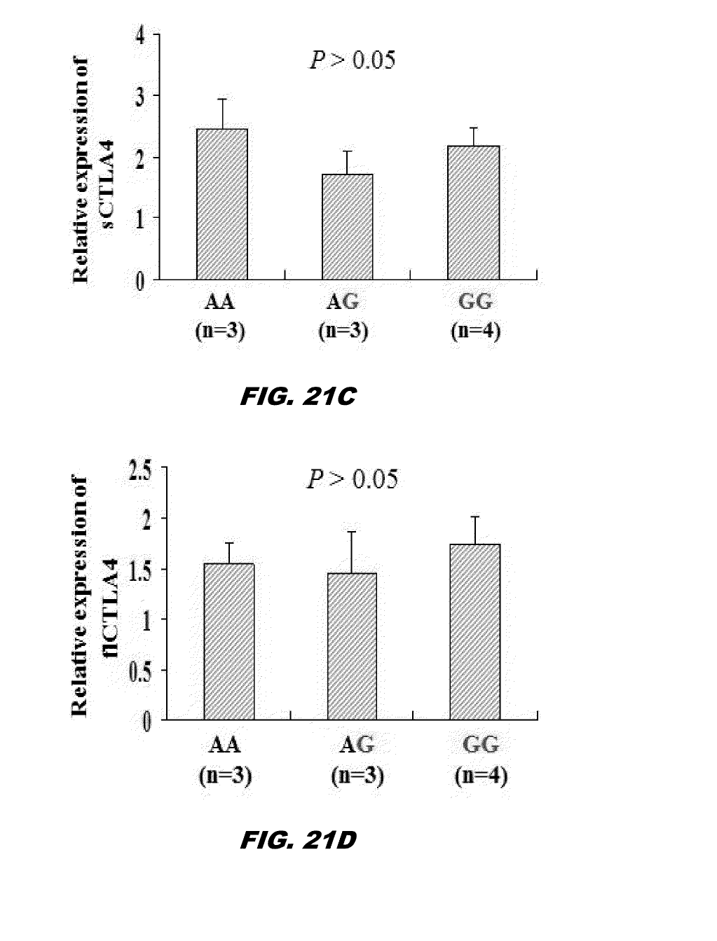

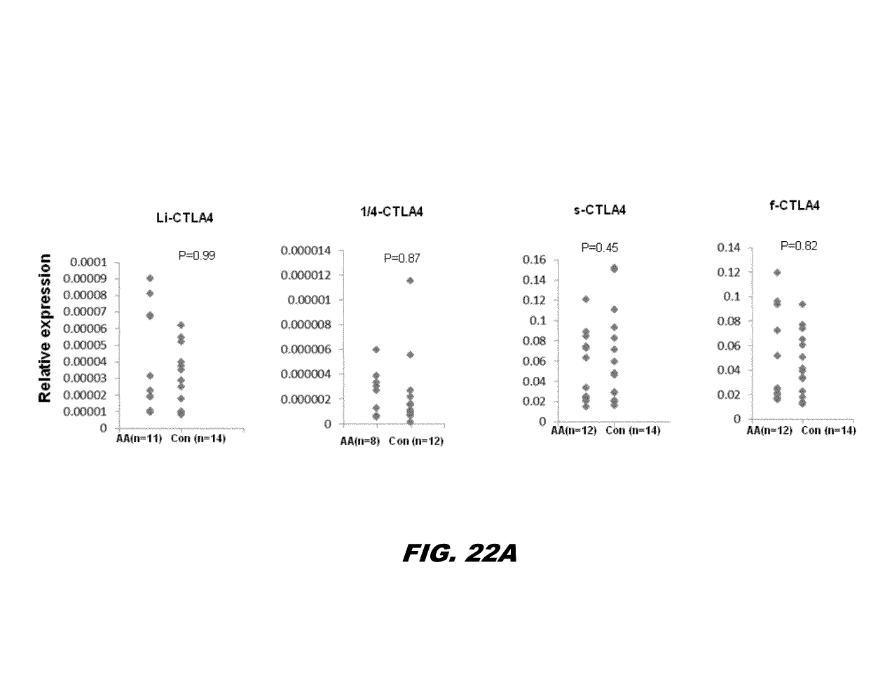

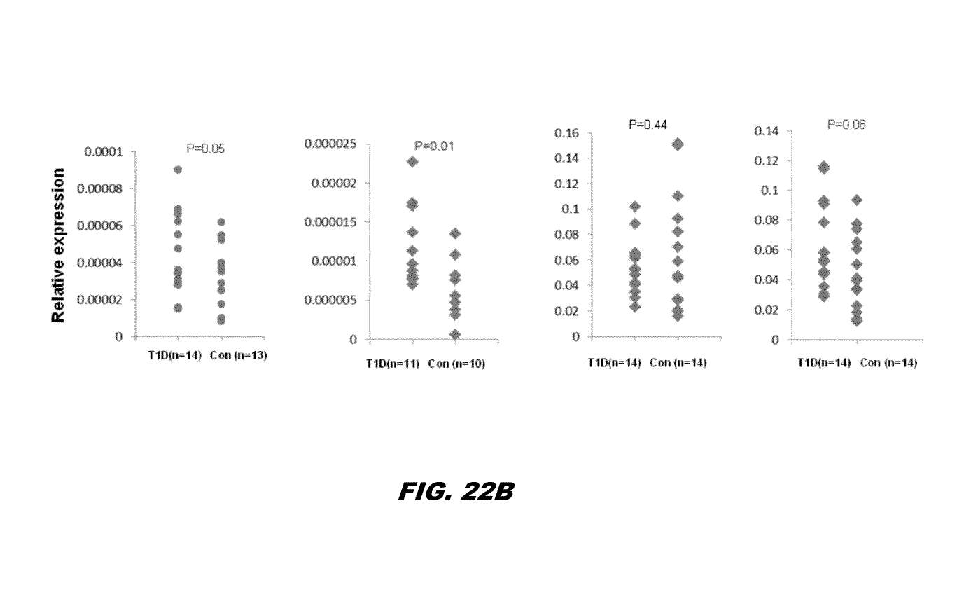

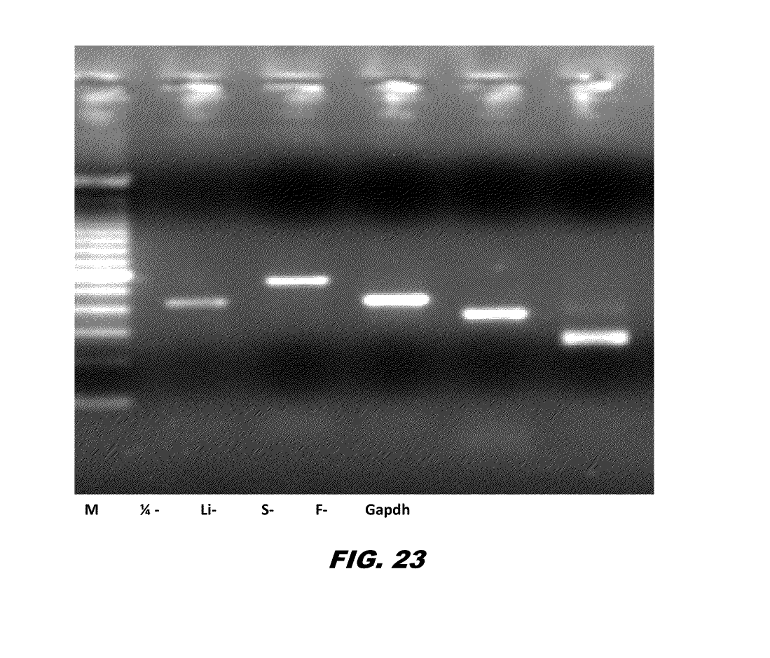

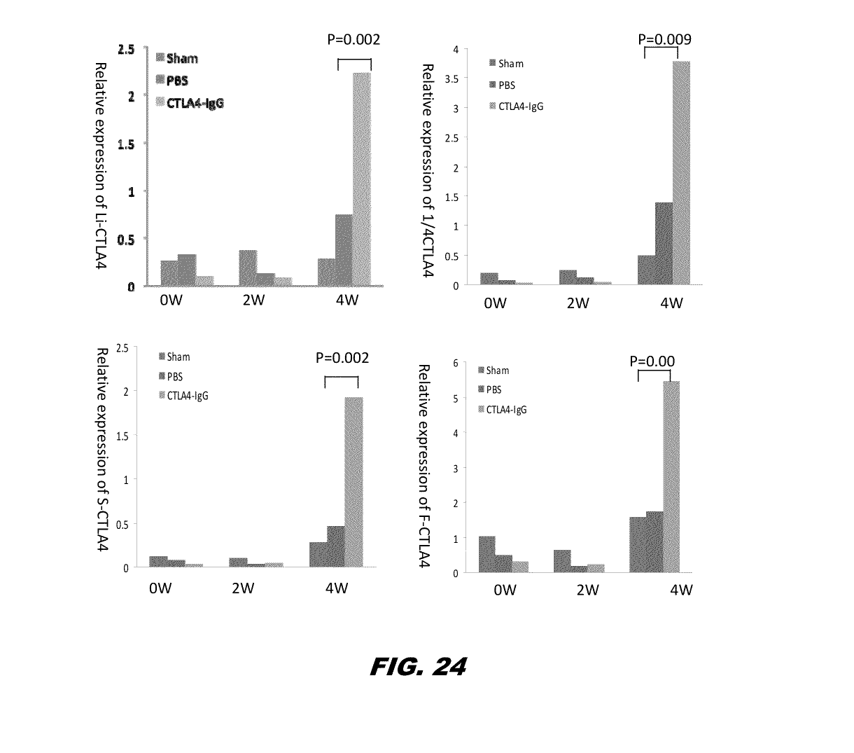

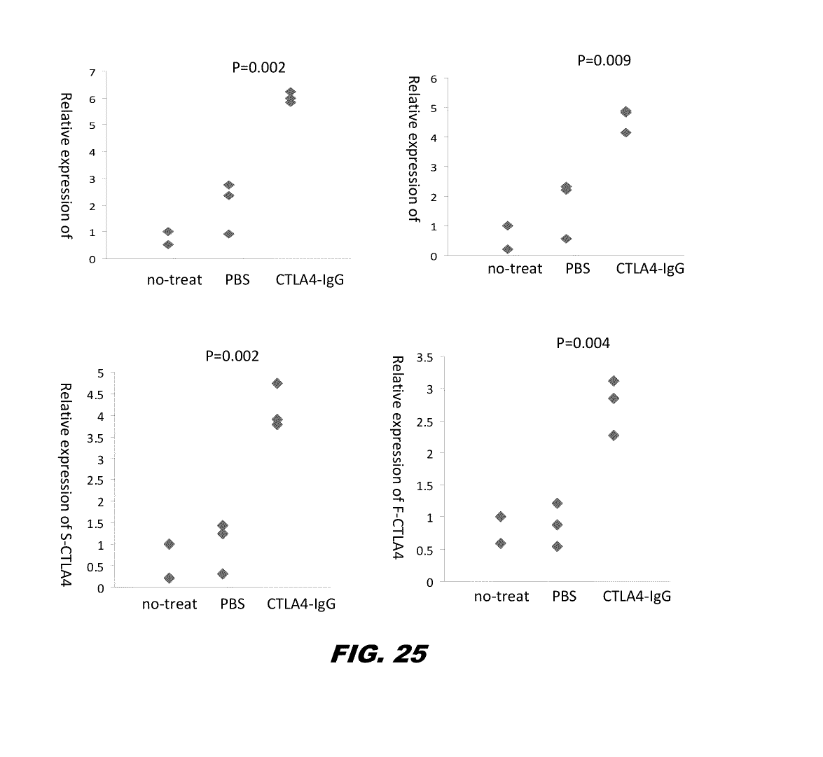

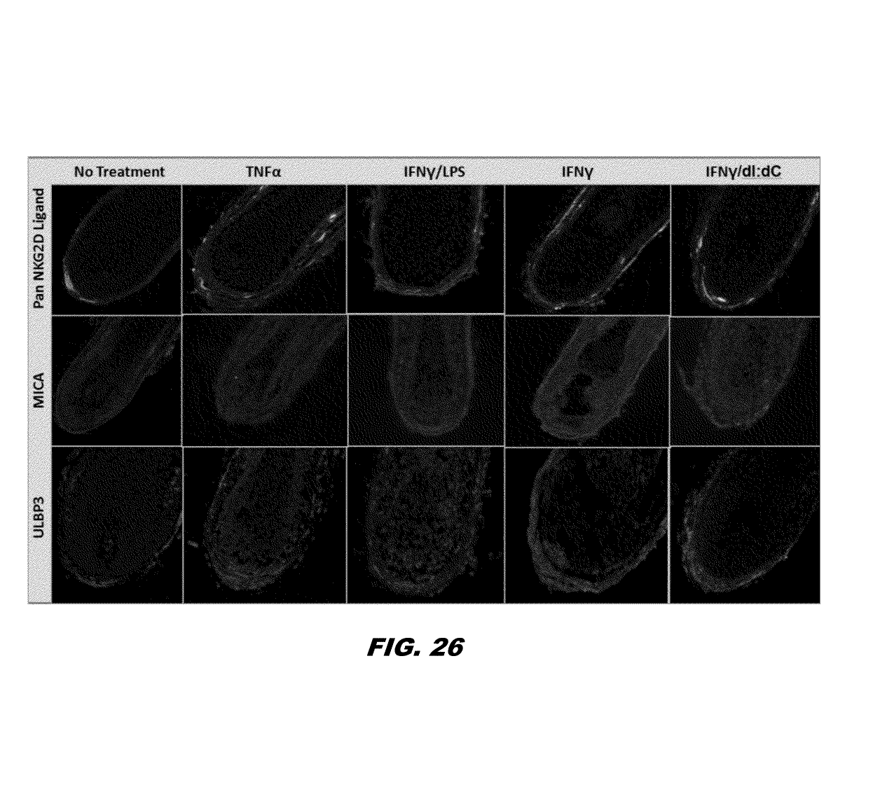

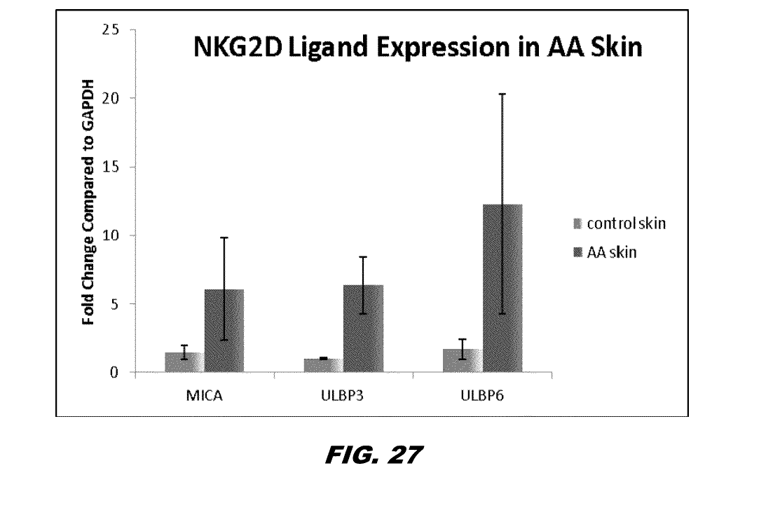

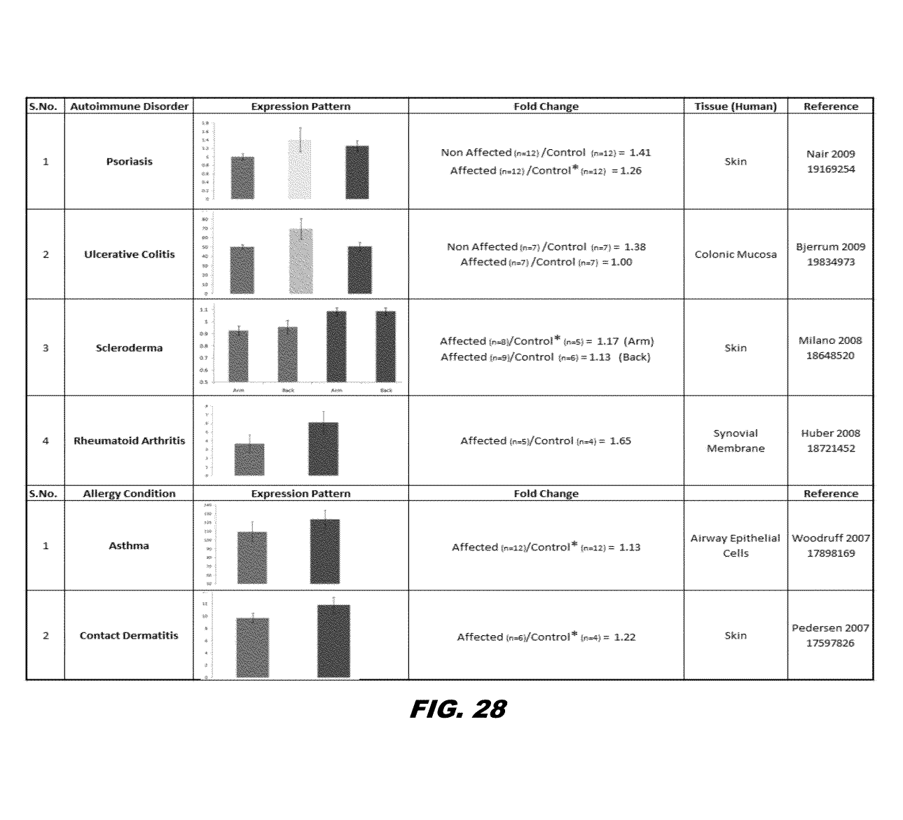

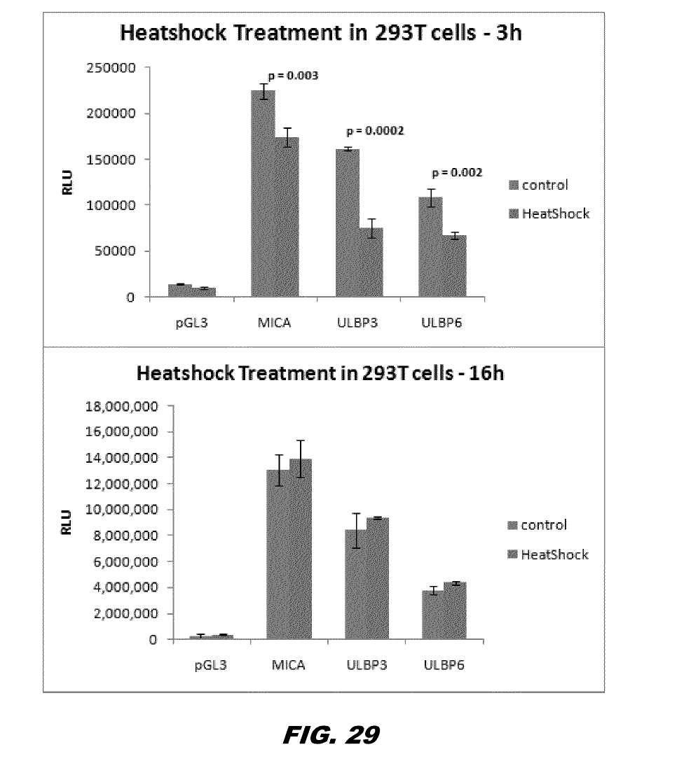

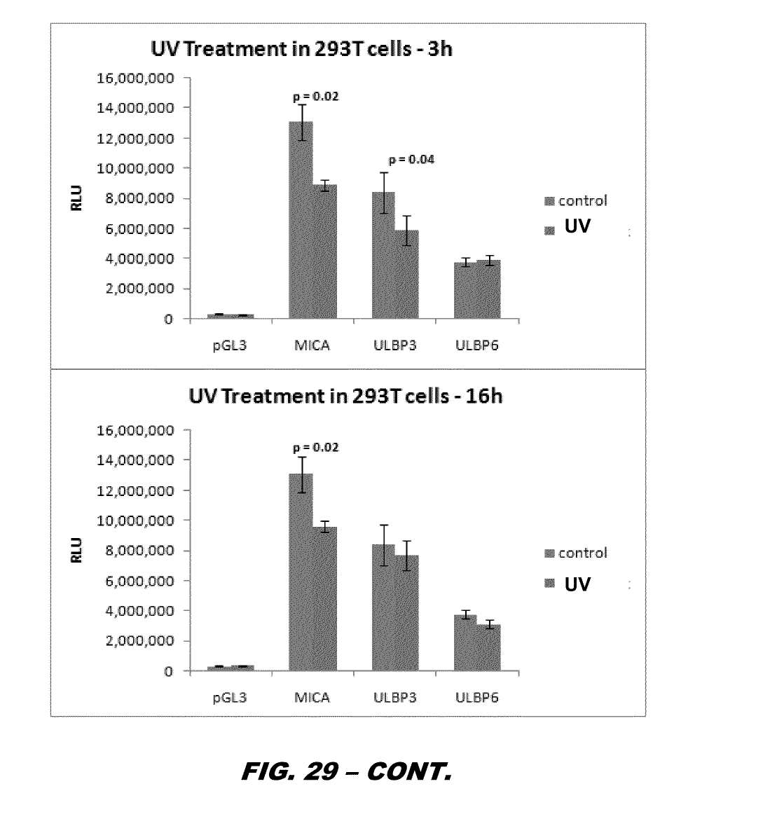

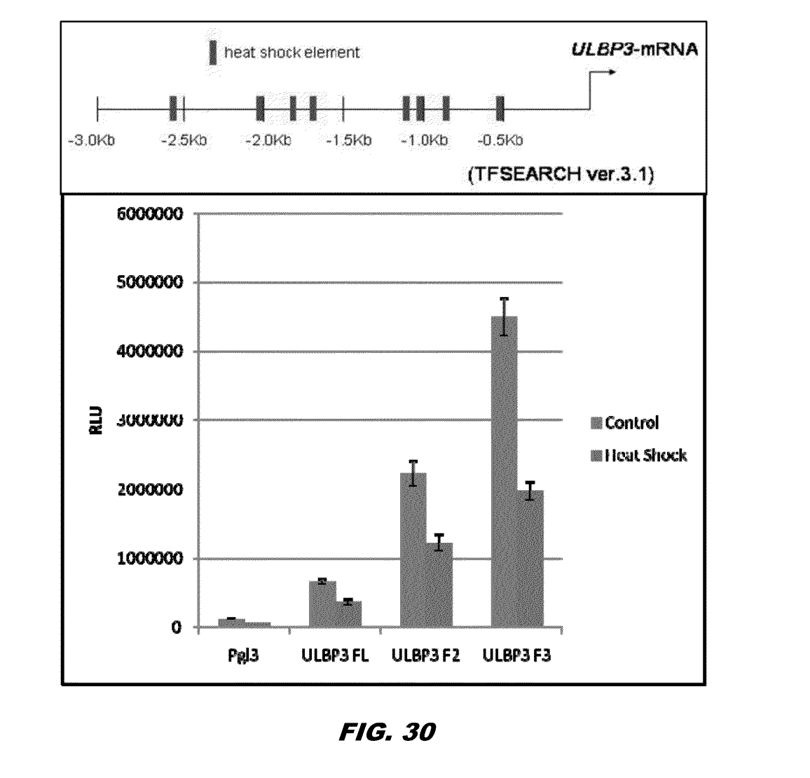

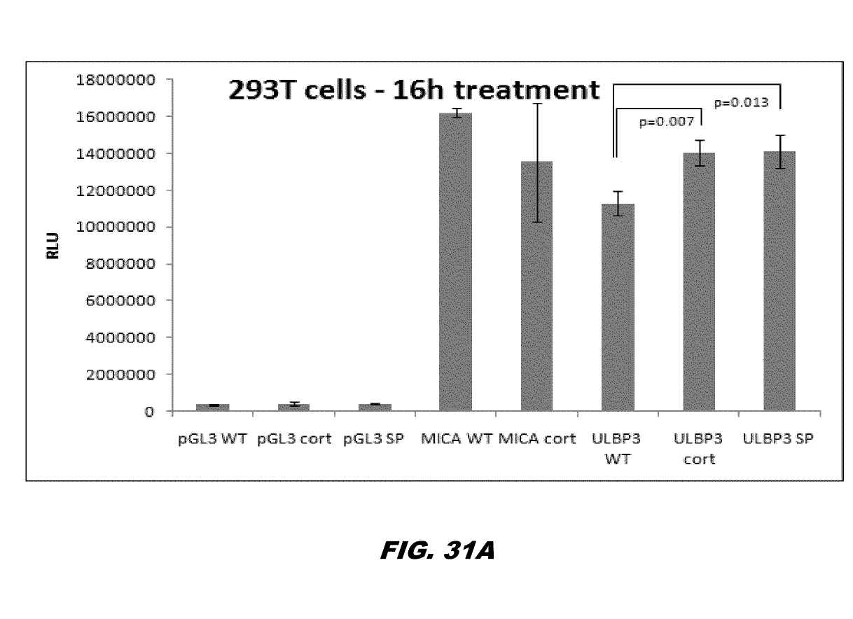

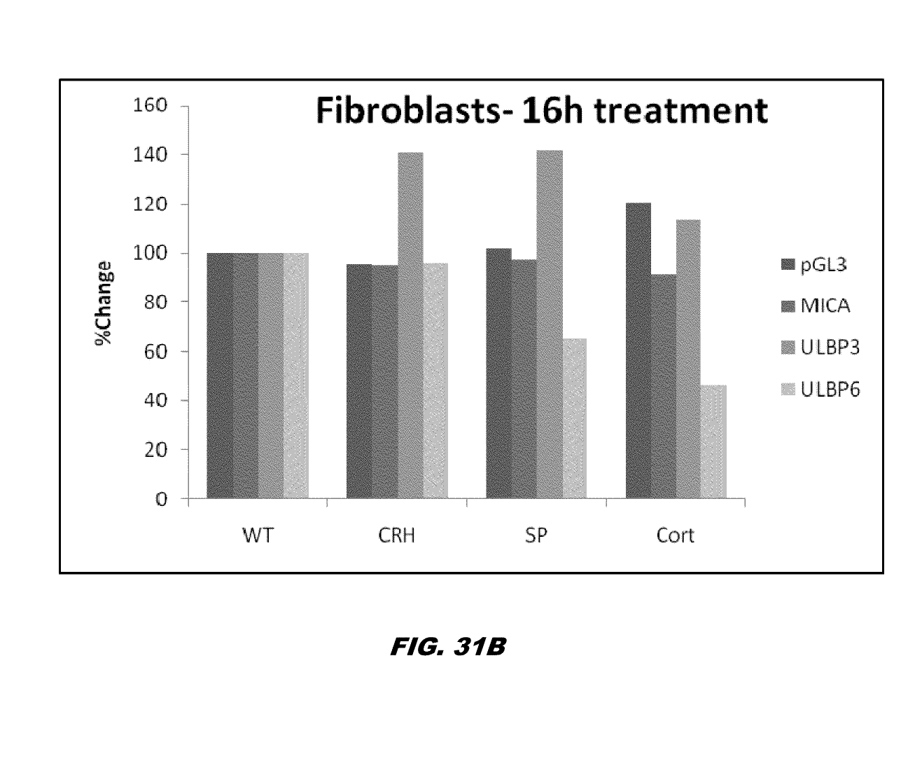

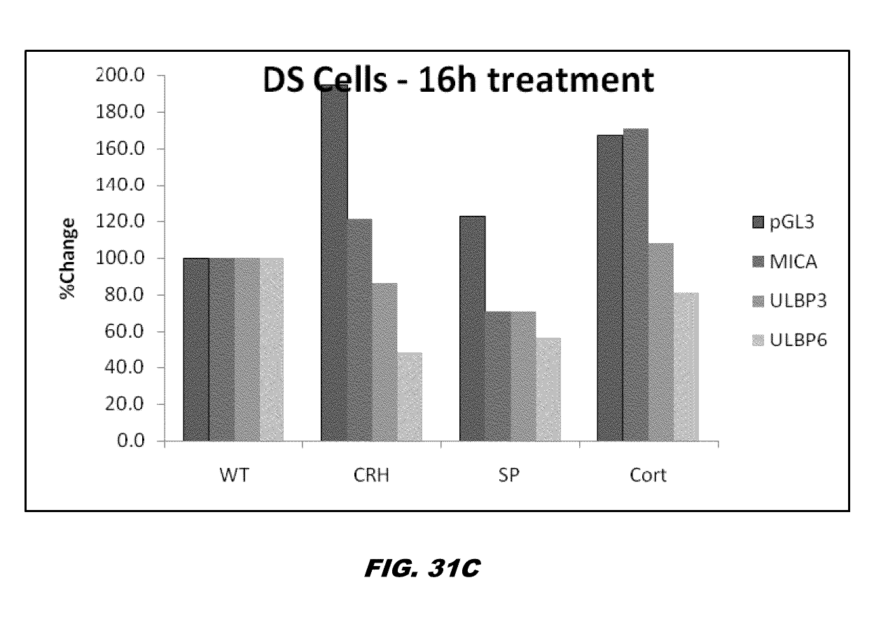

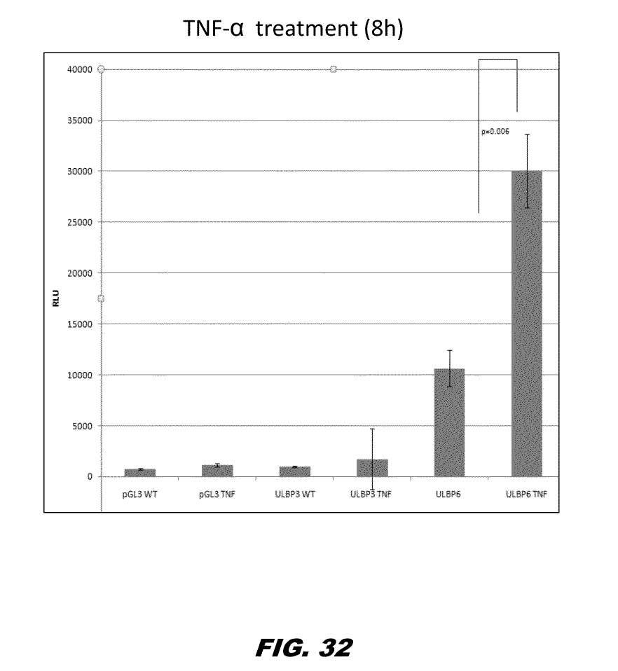

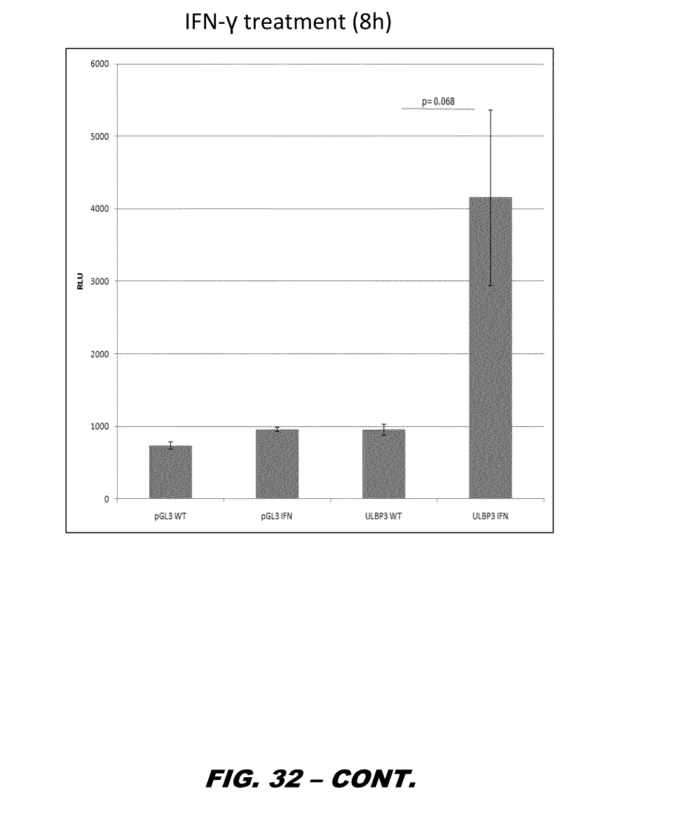

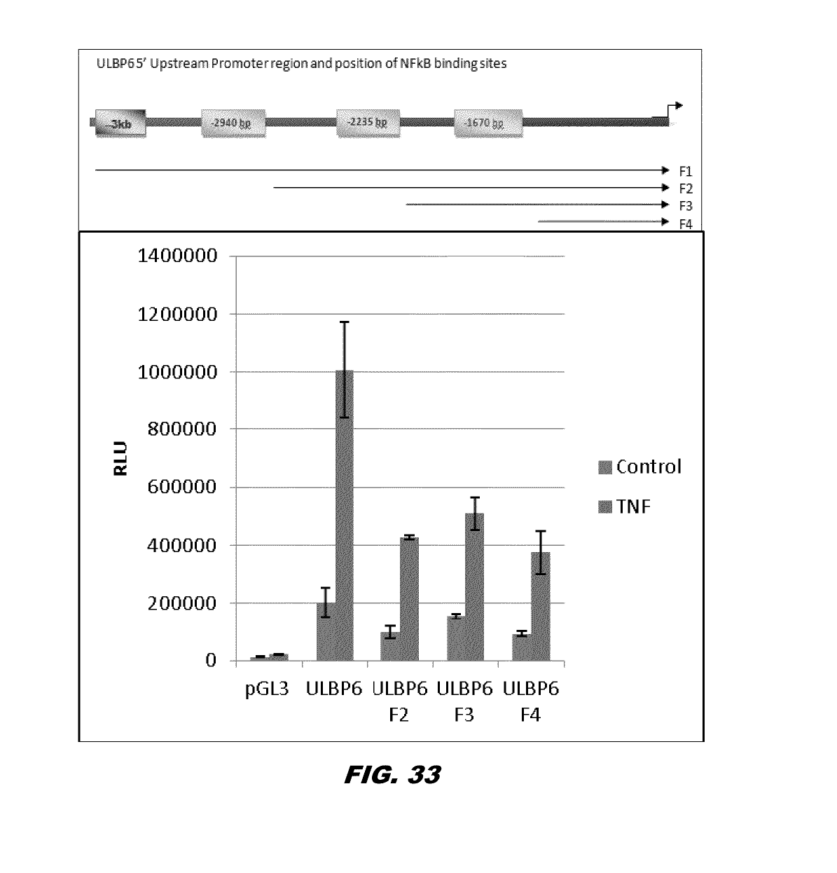

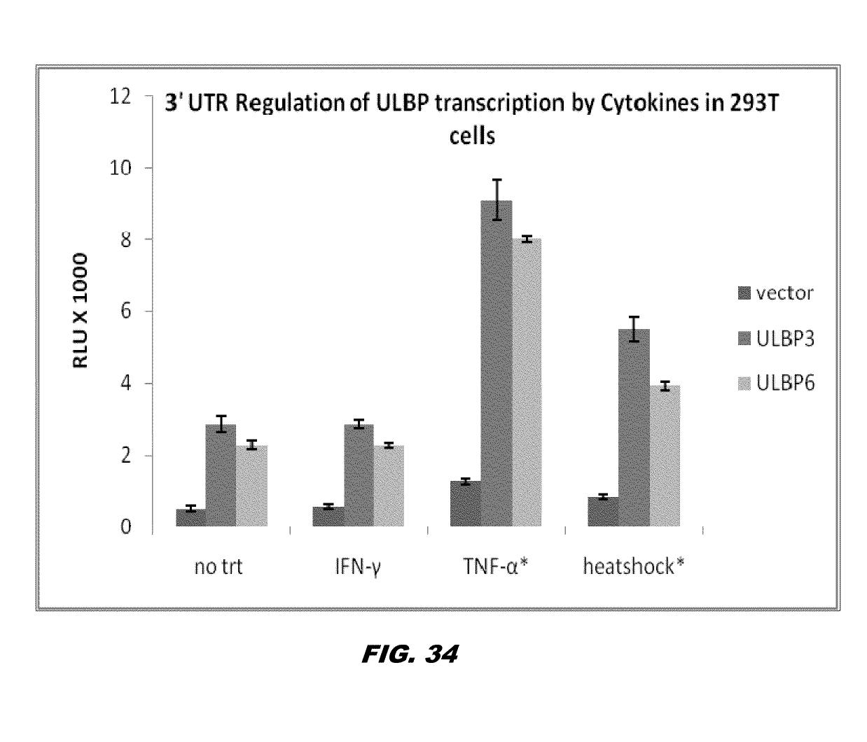

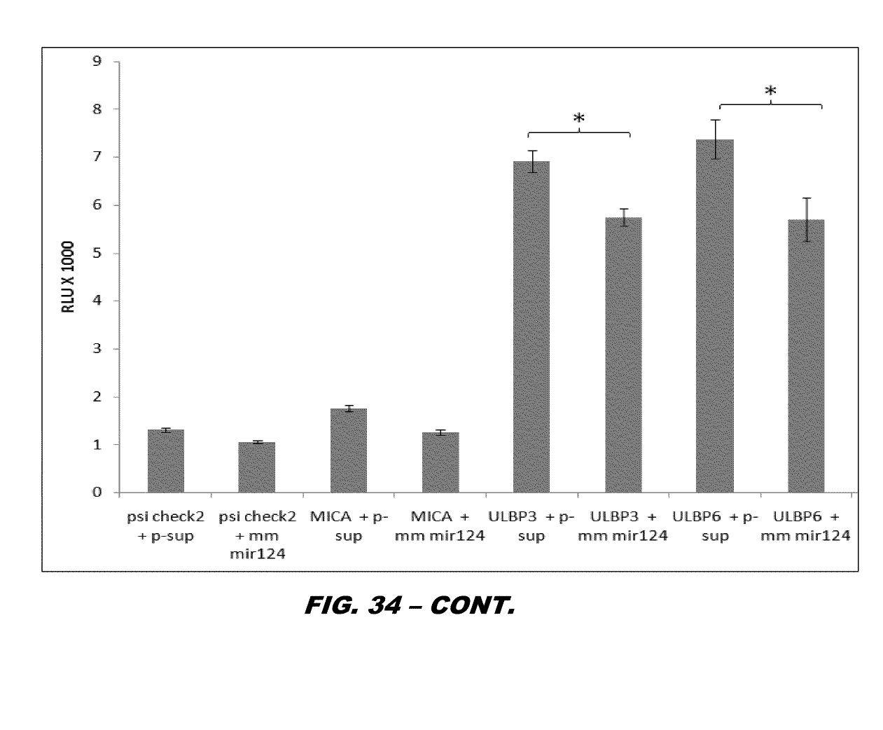

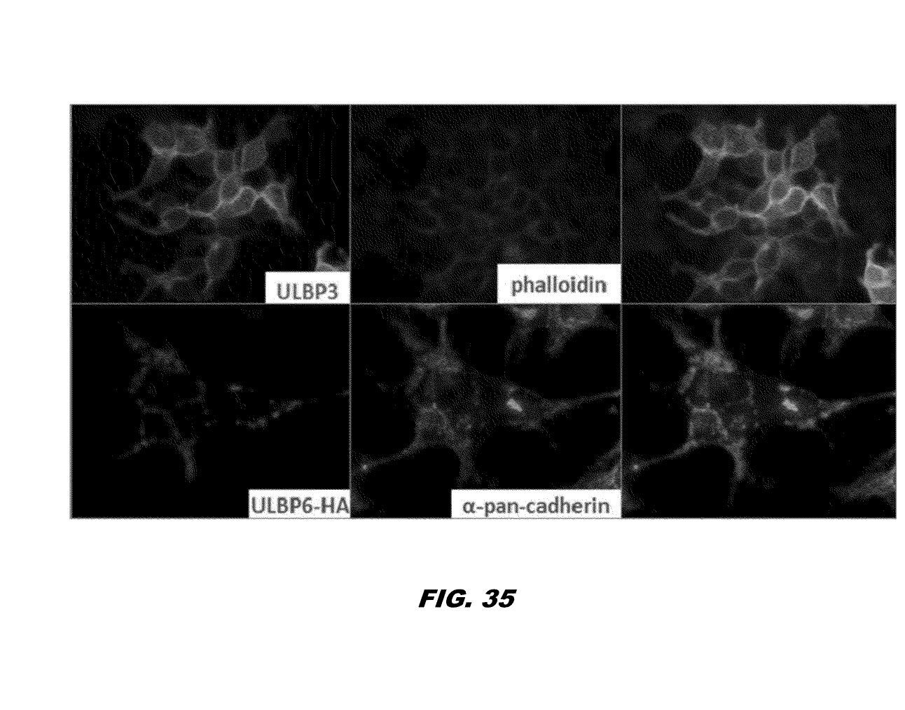

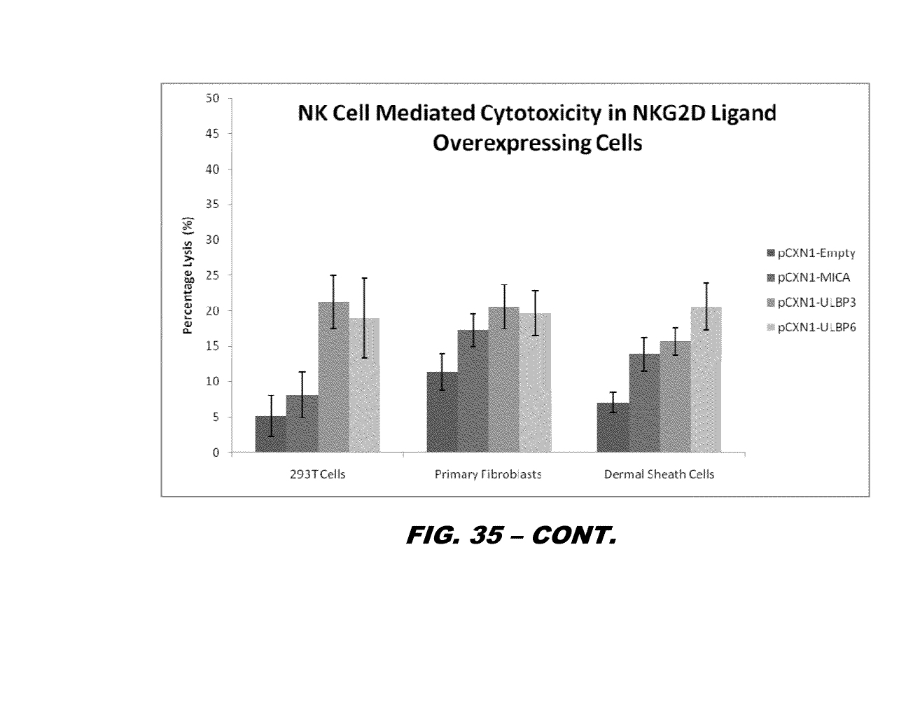

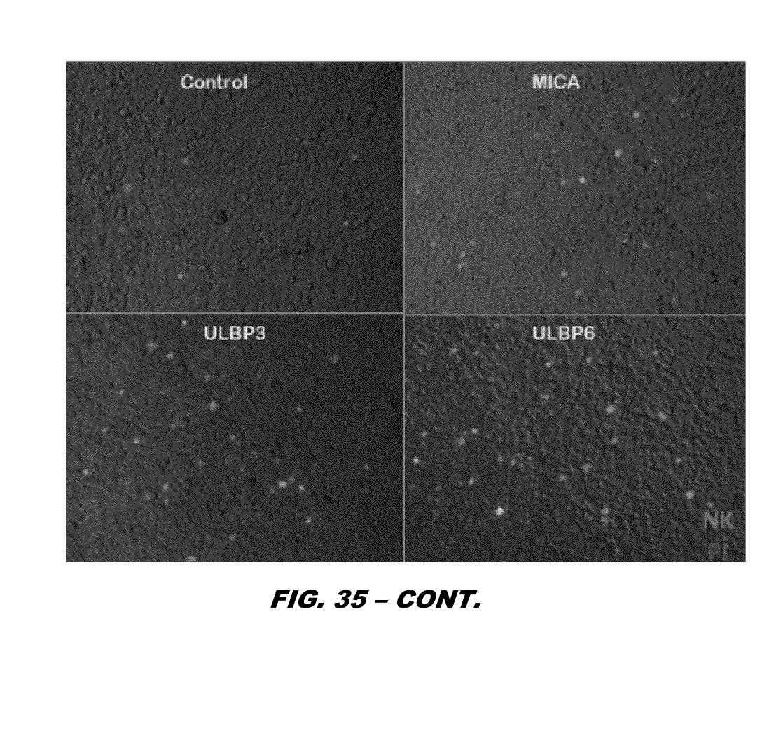

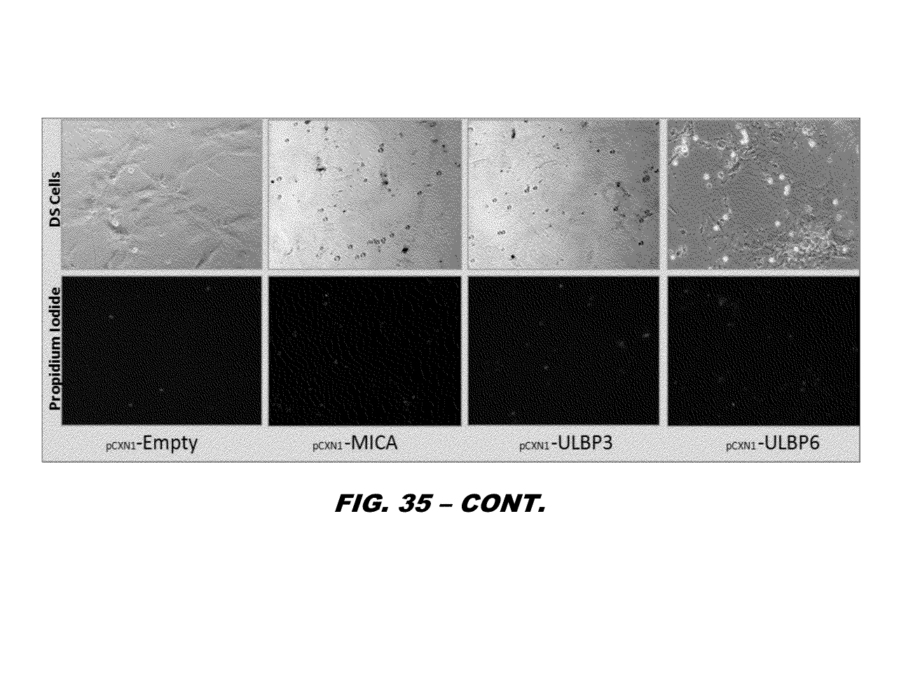

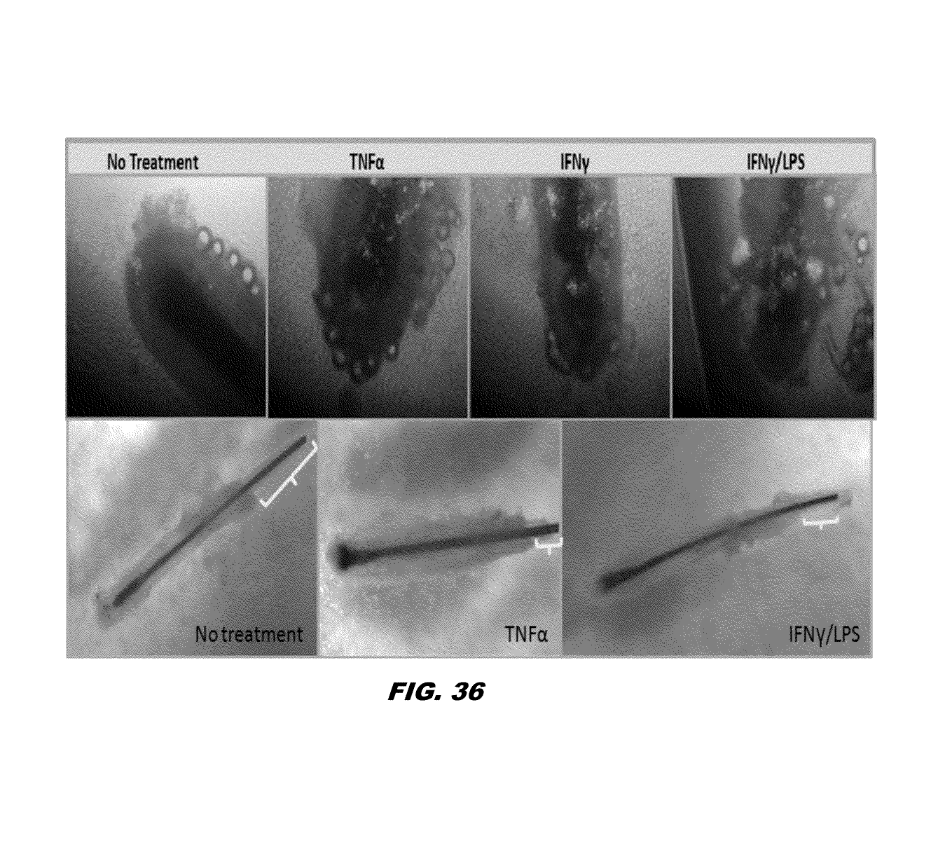

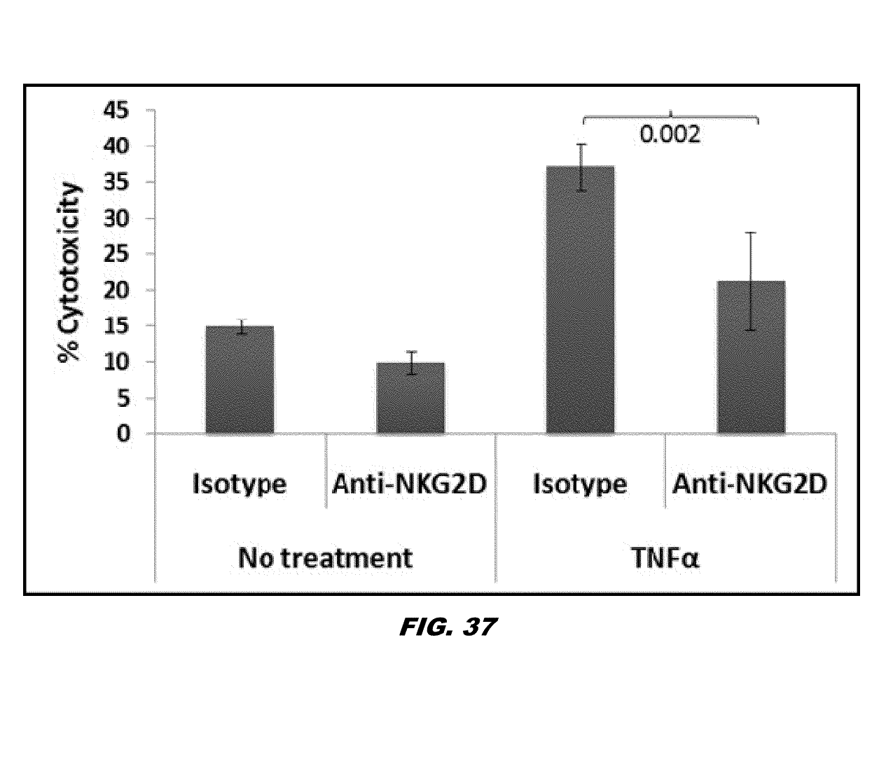

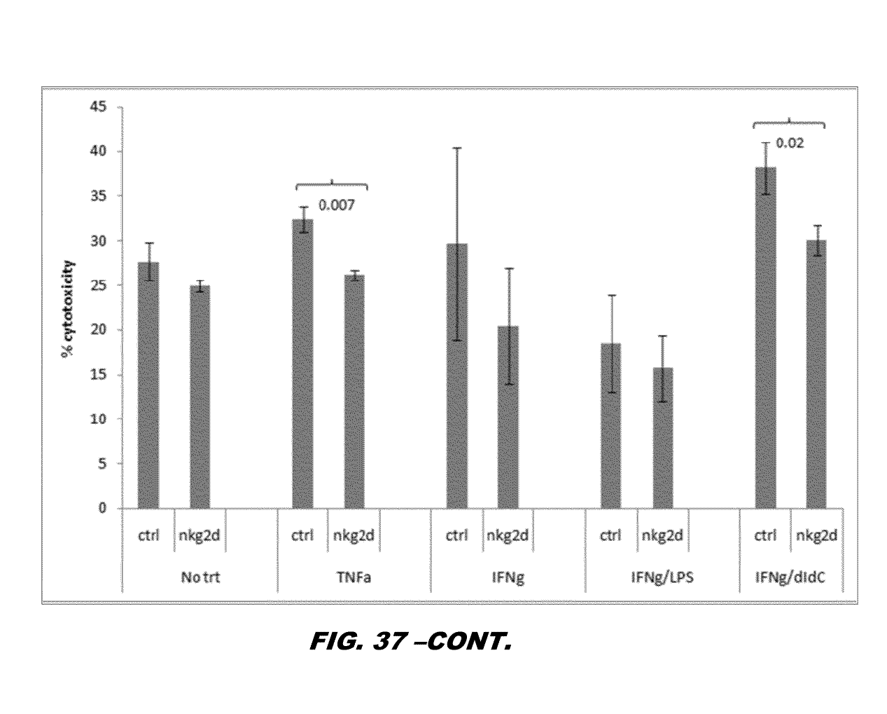

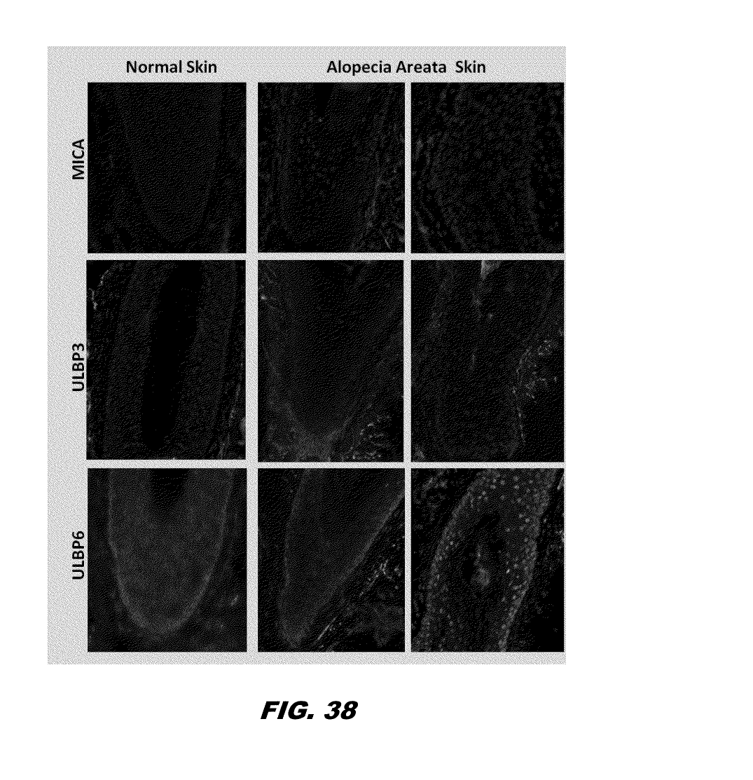

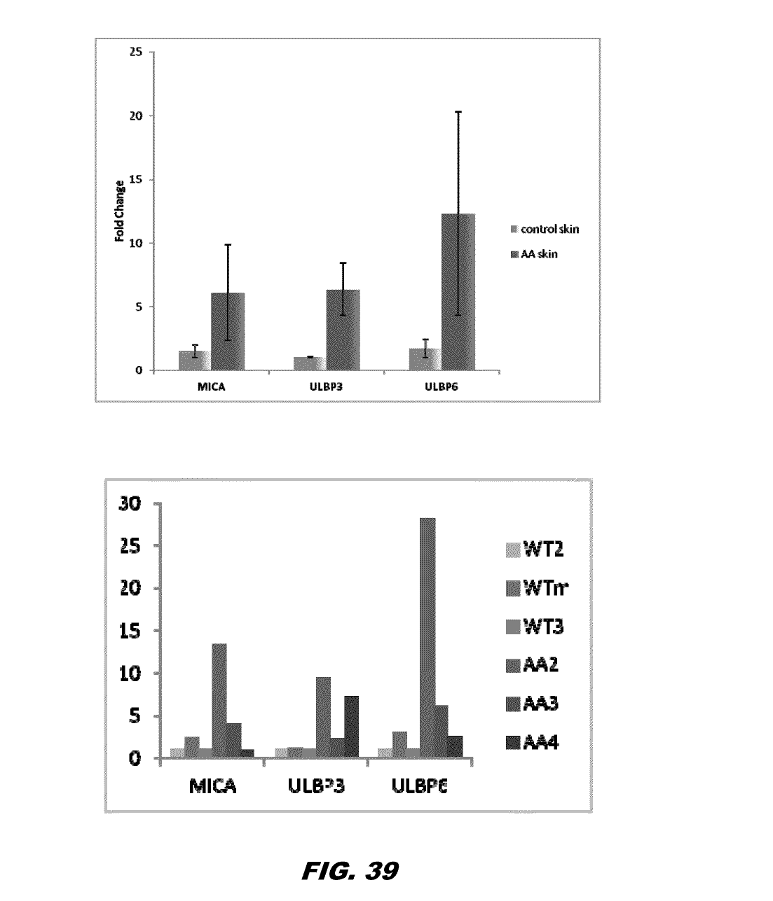

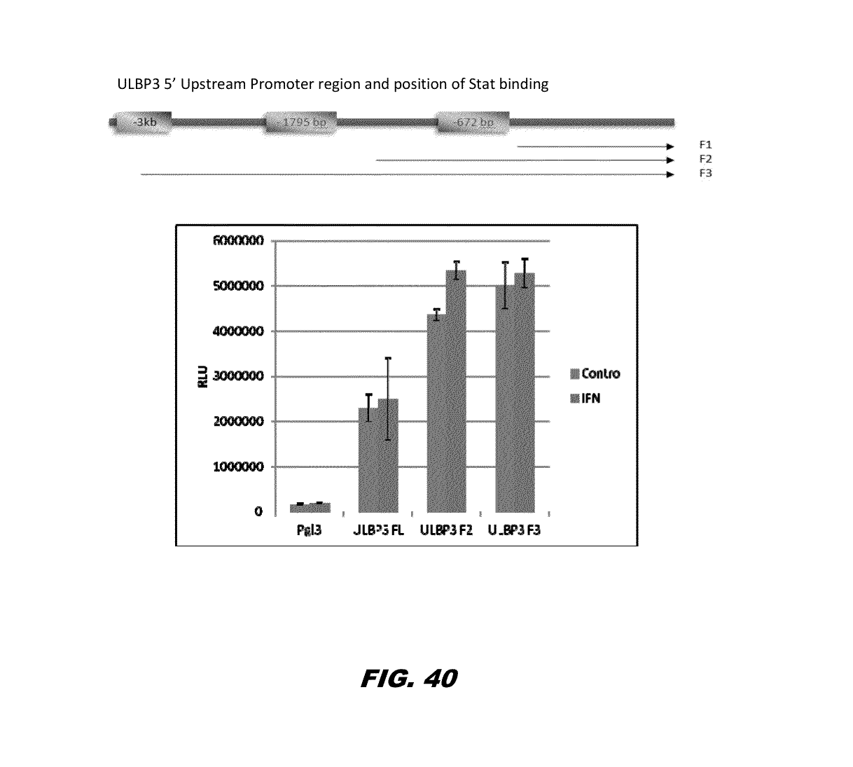

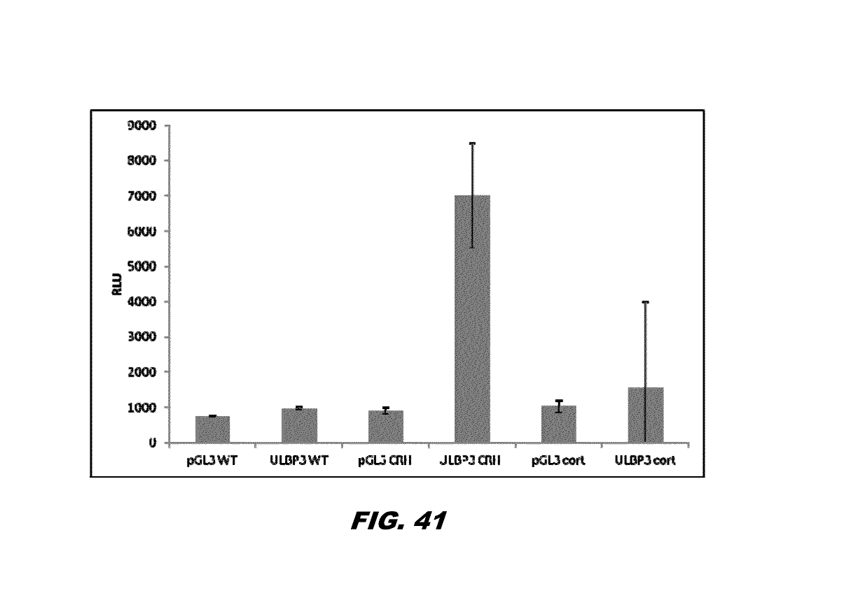

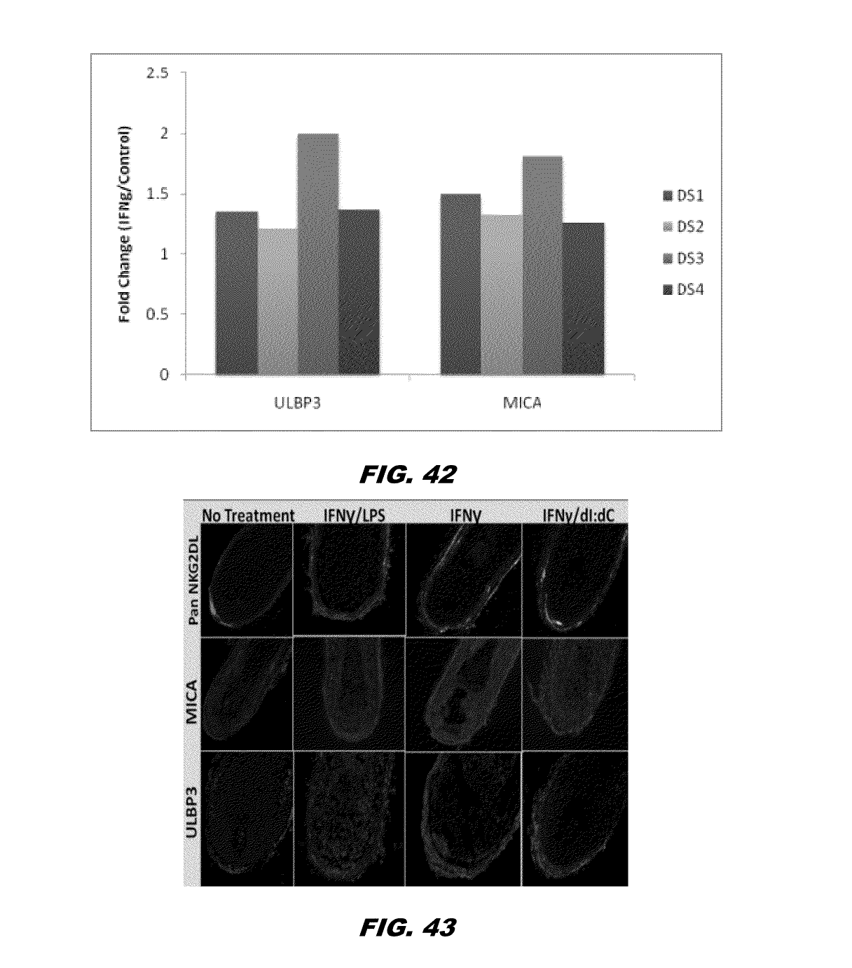

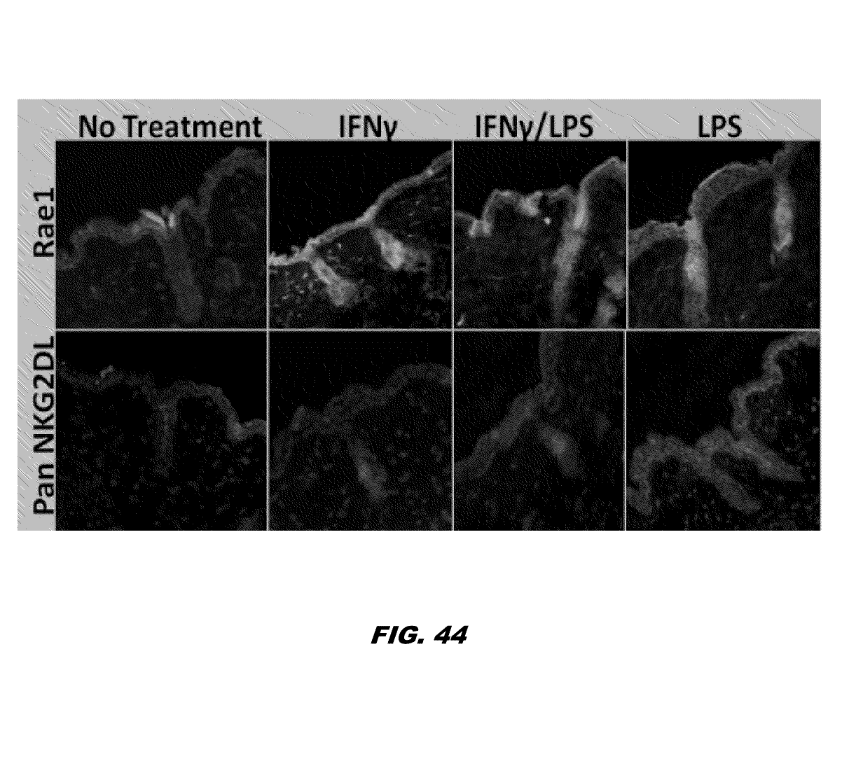

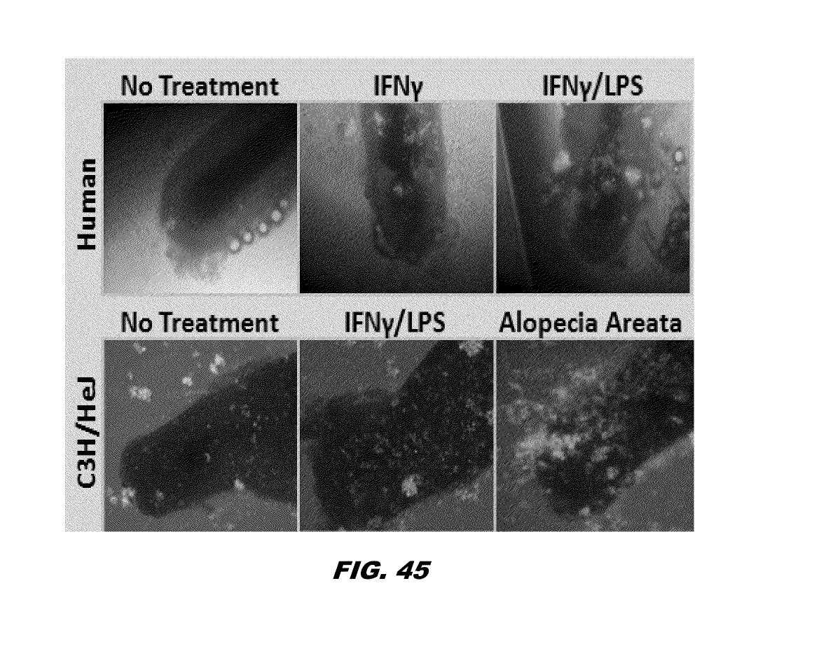

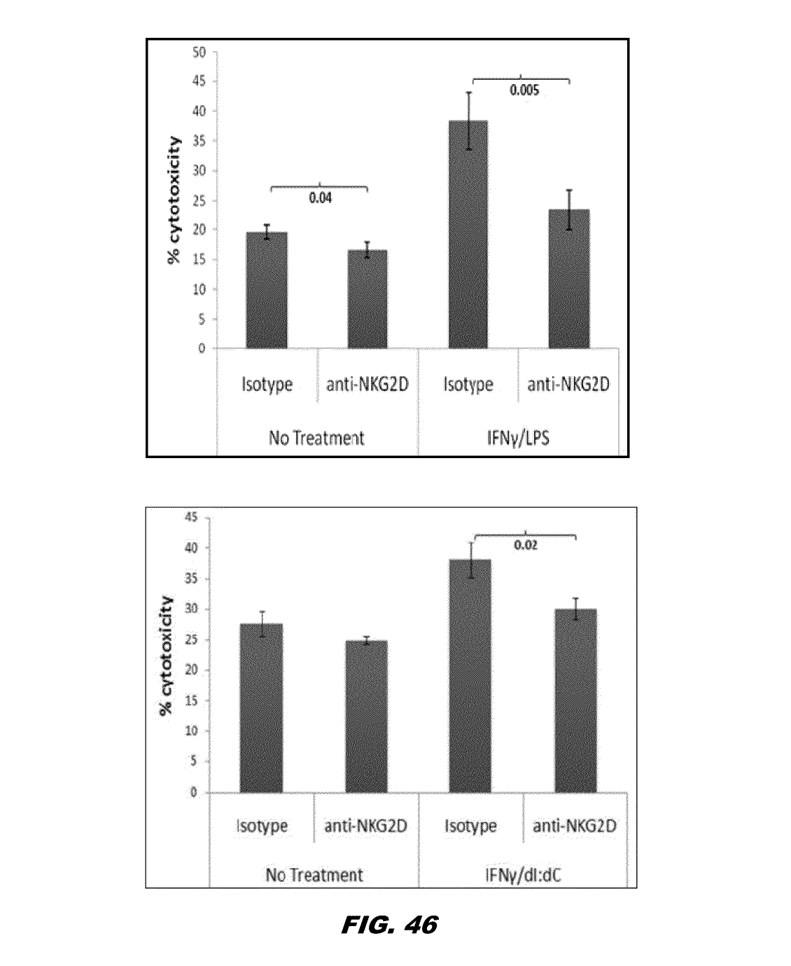

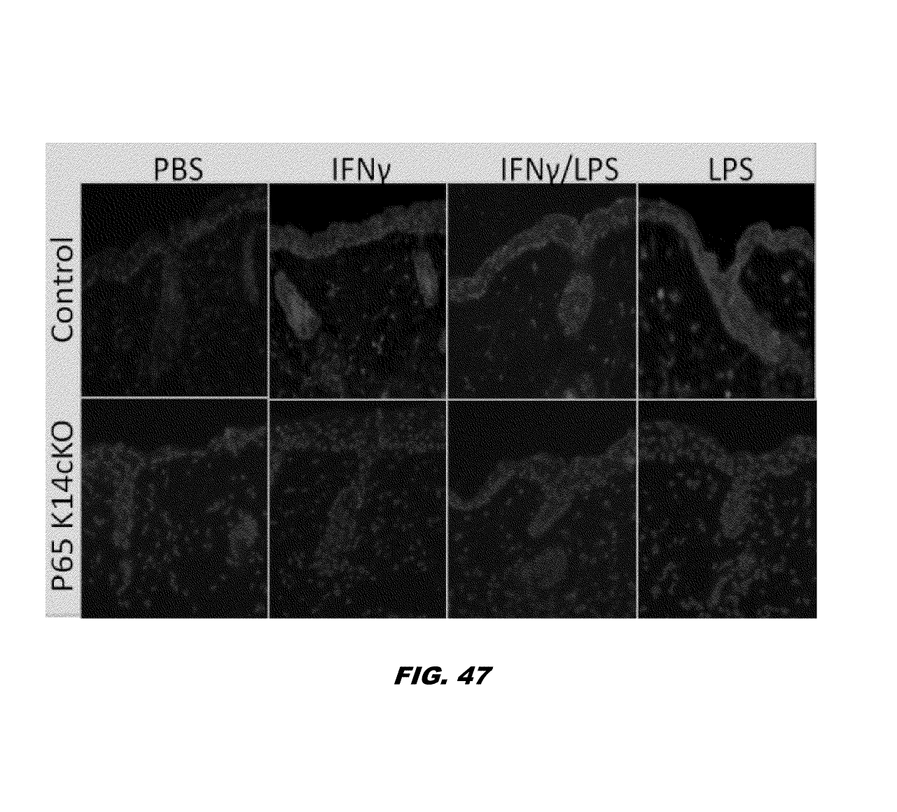

The NKG2D ligands are upregulated under conditions of cellular stress including DNA damage and Toll like receptor (TLR) ligation, all of which are well-known triggers of the NF-κB transcription factor family. Nevertheless, the role of NF-κB in NKG2DL expression has not been thoroughly investigated. One NKG2D ligand, MICA, has been shown to be regulated by NF-κB. For others, a more complex picture of the contribution of NF-κB has emerged. However, to date, there has been no analysis of the transcriptional regulation of the recently reported NKG2D ligand ULBP6. It was discovered that NF-κB activation can directly drive transcription from a ULBP6 promoter (FIG. 17). Furthermore, MICA, ULBP3, and ULBP6 mRNA are upregulated in the AA lesional HF (FIG. 18A). As NKGD2L are known to be regulated post-translationally as well, upregulation of ULBP3 protein was also examined by immunofluorescent microscopy, which demonstrated a more striking increase in ligand expression than was observed by quantitative PCR (QPCR) (FIG. 18B).

Owing to the important role of the NKG2DL-NKG2D axis in both anti-viral immunity and tumor immunosurveillance, understanding the transcriptional regulation of the ULBP family is of substantial interest. While there has been progress in this area, to date, there has not been an intensive investigation of the contribution of NF-κB. An important aspect is taking a targeted approach and dissecting the contribution of a single transcription factor family to the regulation of the expression of NKG2DLs. This approach will allow one to expand the investigation beyond the 5-prime 500 bp “promoter” region that has been the focus of the majority of previous efforts, to include distal and intergenic elements that are likely to also contribute substantially to the regulation of these genes.

Studies have linked activation of NF-κB to the activation of transcription from a ULBP3/6 promoter (FIG. 17), and upregulation of NKG2DL in AA skin (FIG. 18). This analysis will be expanded to additional members of the human and mouse NKG2DL family and the analysis will be extended to additional promoter and enhancer regions of the NKG2DL genes. Furthermore, the binding of the NF-κB family to relevant sites will be investigated by ChIP and contribution of NF-κB will be confirmed using DN constructs in human HF cultures and genetic approaches in mice.

### Example 6

### Expression of MICA and ULBP3

TABLE 17
Transcript expression of MICA and ULBP3 under
conditions of stress in skin components.

| | | DS Cells | | Fibroblasts | | Keratinocytes | |
|---|---|---|---|---|---|---|---|
| | | ULBP3 | MICA | ULBP3 | MICA | ULBP3 | MICA |
| Genotoxic | UV | 1.4 | 1.1 | 1.45* | 1.96* | 1.58* | 1.2 |
| Stress | Hydrogen | 0.55 | 0.95 | 0.96 | 1.25 | 1.01 | 1.27 |
| | Peroxide | | | | | | |
| | Heat Shock | 0.91 | 0.94 | 0.68* | 1.64* | 1.63* | 2.32* |
| Stress | Corticotropin | 1.56 | 0.96 | 0.87 | 1.07 | 1.18 | 0.35 |
| Hormones | Releasing | | | | | | |
| | Hormone (CRH) | | | | | | |
| | Substance P | 0.93 | 1.32 | 0.84 | 0.9 | 0.87 | 0.55 |
| | Hydrocortisone | 0.99 | 1.23 | 0.63 | 1.66 | 0.82 | 0.33 |
| Inflammatory | TNF-α | 0.93 | 1.03 | 0.66 | 2.3 | 0.39 | 0.46 |
| Cytokines | IFN-γ | 1.68 | 1.53 | 0.41 | 2.14 | 0.49 | 0.55 |

NKG2D ligands are responsive to stress stimuli and show upregulation under conditions of stress. Primary cell lines derived from skin and the hair follicle—dermal sheath cells, fibroblasts and keratinocytes were subjected to stress conditions. Genotoxic stress was induced by subjecting the cells to conditions which cause DNA damage and induce ATM/ATR response which is known to signal downstream and affect NKG2D ligand regulation. Cells were given treatment of UVB 300 j/m², hydrogen peroxide 1 mM for 3 hours and heat shock at 42° C. water bath for 1 hours followed by a 2 hr recovery period. Skin is a highly innervated organ wherein the efferent neurons produce various factors associated with the stress response canonically associated with the HPA axis. Primary cells were given 24 hr treatment with the HPA associated stress hormones—corticotropin releasing hormone, substance P and hydrocortisone. Inflammatory cytokines ae produced in the skin in response to damage and infection and are potential inducers of NKG2D ligand expression. The effect of pro-inflammatory cytokines—TNF-α and IFN-γ were assessed on the primary cell cultures.

### Example 7

### NKG2D Ligands and Receptor

### NKG2D Receptor

The presence of both activating receptors and inhibitor receptors maintain a state of equilibrium within the organism. Inhibition of NK cells occurs via MHC I by inhibitory receptors whereas activating receptors such as Ly49H and NKp46 which recognize viral associated antigens trigger the cytotoxic activity. (Bottino, Castriconi et al. 2005) Another class of activating receptors is NKG2D, a cell surface receptor present canonically on the surface of NK, NKT and γδ T-Cells. It is also present on the surface of all human and activated mouse CD8+ve T-cells (Ehrlich, Ogasawara et al. 2005). Interferon producing killer dendritic cells (Chan, Crafton et al. 2006) and a special subset of CD4+ve cells (Dai, Turtle et al. 2009) also express NKG2D on their surface. The receptor gene is coded in humans on chromosome 12 and in mice on chromosome 6 along with other members of the NKG2 natural killer cell receptor family of C-type (Ca2+) lectin like receptors (Yabe, McSherry et al. 1993) which contain NKG2-A, -B, which are splice variants and -C all of which share high degree of homology—94% in extracellular domain and 56% in transmembrane and intracellular domains whereas -D which has a very different amino acid composition and has only 21% sequence homology with others (Houchins, Yabe et al. 1991). NKG2D receptor lacks an intracellular signaling domain and requires the adaptor protein DAP10 for downstream signal transduction. It exists in a hexameric complex on the cell membrane (Wu, Song et al. 1999). High degree of homology between NKG2D receptor in humans and mice is observed and these show cross species reactivity (ULBP1 and 2) (Sutherland, Rabinovich et al. 2006).

NKG2D Ligands:

NKG2D receptor shows promiscuous binding to a variety of ligands belonging to the non classical members of the MHC superfamily with MHC class-I like α1 α2 receptor binding domains. Two classes of NKG2D are present in humans donated as MIC (A and B) and the ULBP (1-6) family and three in mice—Rae1 (α-ε){retinoic acid early inducible}, H60 {histocompatibility antigen 60} and Multi {murine ULBP-like transcript 1}. The Families differ in their structure, chromosomal position and sequence. MICA and B are transmembrane protein, have an extra α3 domain but do not associate with bta-2 microglobulin. MIC genes are present on chromosome 6 within the MHC cluster. ULBP proteins are also present on chromosome 6 but do not map to the MHC cluster. ULBP 1-3 and 6 are GPI anchored proteins whereas ULBP4 and 5 have transmembrane domain. In mice—Rae1 have GPI anchors where as Multi and 1-160 have transmembrane domains. (summarized in review) (Eagle and Trowsdale 2007)

The degree of allelic polymorphism observed in NKG2D ligands in general population is very high, and is increasingly being associated with disease and pathology. MICA is known to have more than 65 alleles which reside mostly in exon 2-4 encoding the extracellular domain of the proteins (Choy and Phipps). Similar genetic polymorphisms—different SNP frequencies and haplotypes have also been observed in the ULBP genes and are associated with different ethnic backgrounds (Afro-Caribbean, Euro-Caucasoid and Indo-Asian) (Antoun, Jobson et al.). In this study, highest polymorphism was observed in ULBP6, ULBP3 and ULBP4—which interestingly shows a skin specific expression. Similar variation in copy number of ULBP genes is also observed phylogeneticaly, with only 6 genes in humans but almost 30 in cattle (11 transcribed) (Larson, Marron et al. 2006). An NKG2D ligand like molecule Mill was also identified in the marsupial opossum, indicating early origin of NKG2D receptor-ligand interaction system. Comparative sequence analysis of the human, cattle, rat, mouse, and opossum genomes explain the high numbers of related ULBP family members through duplication and subsequent divergence events (Kondo, Maruoka et al.). Structural differences in the NKG2D ligands confer differential binding affinities as well as compartmentalization. All NKG2D ligands interact with the receptor via their α1-α2 domain and the kinetics of these interactions are determined by the amino acid sequence of the binding domain (McFarland and Strong 2003). In mice both rae 1 family and H60 compete for the receptor but H60 shows more than 25 fold higher binding affinity (O'Callaghan, Cerwenka et al. 2001). The membrane bound NKG2D ligands especially GPI anchored ULBPs tend to accumulate within lipid rafts which occur at the immune synapse between target and effector cells. MICA shows S-acylation which also confers weak raft targeting properties (Eleme, Taner et al. 2004). Polymorphisms in the cytoplasmic tail of MICA lead to differential targeting to basolateral or apical surface of epithelial cells. (Suemizu, Radosavljevic et al. 2002).

Regulation:

NKG2D ligands act as a first line of defense alerting the innate immune system of the presence of aberrant or transformed cells. Both human and murine ligands show induction after viral infections such as cytomegalovirus, HTLV-1, HIV (Wilkinson, Tomasec et al. 2008), (Azimi, Jacobson et al. 2006; Ward, Bonaparte et al. 2007). NKG2D ligands also show increased expression on tumors. Dysregulation of ULBP proteins is commonly observed in cancers such as laryngeal squamous cell carcinoma and colorectal cancer (Chen, Xu et al. 2008), (McGilvray, Eagle et al. 2009). To avert the detection of malignancy, tumors often shed extracellular domains of NKG2D ligands by proteolytic cleavage by metalloproteases or by exososomal release, which causes elevated levels of soluble ligand in the blood (Fernandez-Messina, Ashiru et al.). Interestingly several cancer studies have shown NKG2D ligands to be good prognostic markers for disease progression such as ULBP2 and ULBP4 for ovarian cancer and soluble ULBP2 for melanoma (McGilvray, Eagle et al.) (Paschen, Sucker et al. 2009). This ligand upregulation is caused due to activation of DNA Damage pathways and oncogenic pathways (Gasser, Orsulic et al. 2005; Boissel, Rea et al. 2006). Presence of NKG2D ligands on ES cells has been described and implicated in prevention of teratomas (Dressel, Schindehutte et al. 2008). Stressors which cause cellular damage such as heat shock, oxidative stress or pharmacological agents such as (proteasome inhibitors, HDAC inhibitors—trichostatin A, valproic acids and cisplatin) induce NKG2D expression as does Retinoic acid which is involved in embryonic developmental. Some of the normal tissues such as epithelial cells, neurons and embryonic tissues express NKG2D ligands constitutively. (Eagle, Jafferji et al. 2009).

The surface expression of NKG2D ligands is also regulated at a transcriptional and post translational level. At a transcriptional level the promoter regions of the ligands contain different putative transcription factor binding sites influencing differential tissue specific expression as well as regulation under stress (Eagle, Traherne et al. 2006). A number of microRNAs have also been shown to bind the 3′UTR of MIC genes and inhibit the transcript levels of the ligands (Stern-Ginossar, Gur et al. 2008). At a post translational level, normal cells which sequester the NKG2D ligands within the cell express the ligands at cellular surface in response to cellular stress (Borchers, Harris et al. 2006).

Role in Autoimmunity:

NKG2D functions to eliminate the aberrant self cells and dysregulation of this recognition process often leads to development of autoimmunity disorders (Van Belle and von Herrath 2009). In rheumatoid arthritis patients, greater numbers of circulating as well as resident CD4 positive cells express NKG2D ligand. These Helper T-cells exhibit a cytotoxic profile with secretion of IFNg, perforin, granzyme B and cytolytic ability. The synoviocytes in RA also secrete soluble MICA into the synovial fluid. (Groh, Bruhl et al. 2003). Crohn's disease patients exhibit elevated MICA staining in the lamina propria as well as a CD4 positive cells which express NKG2D receptor, secrete IFNg and perforin and are cytolytic (Allez, Tieng et al. 2007). MICA levels were found to be upregulated in active cases of celiac disease which lower with gluten free diet, along with higher soluble MICA concentrations in the patient's sera. Elevated NKG2D density was observed on intraepithelial lymphocytes of patients along with more efficient NKG2D facilitated cytotoxic response against epithelial cells (Hue, Mention et al. 2004). Non Obese diabetic mice are used as a model of type 1 diabetes in humans. A study done in these mice elucidates the importance of NKG2D receptor engagement in the development of pancreatic β-cell autoimmunity. The levels of Rae1—the murine NKG2D ligand were elevated in NOD mice compared to control balb/c mice and exhibited progressive increase with age in NOD as well as NOD SCID mice indicating that elevation of rae 1 is independent of immune response. Interestingly, NKG2D neutralizing antibody treatment in NOD mice prevented the development of T1D, underscoring the importance of NKG2D pathway in the development of autoimmunity (Ogasawara, Hamerman et al. 2004). In cases of multiple sclerosis as well elevated MICB serum levels were associated with disease relapse (Fernandez-Morera, Rodriguez-Rodero et al. 2008). Interestingly, a study also demonstrated an elevation of MICA ligand in the hair follicle of alopecia areata along with infiltration of the peribulbular tissue with NKG2D+ve CD8 and NK cells. NKG2D as well as NKG2C density were higher in NK cell of AA patients (Ito, Ito et al. 2008). The involvement of ULBP family of NKG2D ligands in the pathogenesis of the autoimmune disease—alopecia areata was shown for the first time through the GWAS Data. Allelic polymorphisms in NKG2D ligands are increasingly being associated with various autoimmune disorders. Specific MICA alleles are overrepresented in rheumatoid arthritis, inflammatory bowel disease and T1D diabetes patients implicating their role in disease pathogenesis (Kirsten, Petit-Teixeira et al. 2009), (Lopez-Hernandez, Valdes et al.) (Gambelunghe, Brozzetti et al. 2007). MICB polymorphisms are also associated with celiac disease, ulcerative colitis and multiple sclerosis (Li, Xia et al.), (Fernandez-Morera, Rodriguez-Rodero et al. 2008), (Rodriguez-Rodero, Rodrigo et al. 2006).

### REFERENCES

Allez, M., V. Tieng, et al. (2007). “CD4+NKG2D+ T cells in Crohn's disease mediate inflammatory and cytotoxic responses through MICA interactions.” Gastroenterology 132(7): 2346-58.
Antoun, A., S. Jobson, et al. “Single nucleotide polymorphism analysis of the NKG2D ligand cluster on the long arm of chromosome 6: Extensive polymorphisms and evidence of diversity between human populations.” Hum Immunol.
Azimi, N., S. Jacobson, et al. (2006). “Immunostimulation by induced expression of NKG2D and its MIC ligands in HTLV-1-associated neurologic disease.” Immunogenetics 58(4): 252-8.
Boissel, N., D. Rea, et al. (2006). “BCR/ABL oncogene directly controls MHC class I chain-related molecule A expression in chronic myelogenous leukemia.” J Immunol 176(8): 5108-16.
Borchers, M. T., N. L. Harris, et al. (2006). “NKG2D ligands are expressed on stressed human airway epithelial cells.” Am J Physiol Lung Cell Mol Physiol 291(2): L222-31.
Bottino, C., R. Castriconi, et al. (2005). “Cellular ligands of activating NK receptors.” Trends Immunol 26(4): 221-6.
Chan, C. W., E. Crafton, et al. (2006). “Interferon-producing killer dendritic cells provide a link between innate and adaptive immunity.” Nat Med 12(2): 207-13.
Chen, X. M., X. Q. Xu, et al. (2008). “NKG2D ligands expression and NKG2D-mediated cytotoxicity in human laryngeal squamous carcinoma cells.” Scand J Immunol 67(5): 441-7.
Choy, M. K. and M. E. Phipps “MICA polymorphism: biology and importance in immunity and disease.” Trends Mol Med 16(3): 97-106.
Dai, Z., C. J. Turtle, et al. (2009). “Normally occurring NKG2D+CD4+ T cells are immunosuppressive and inversely correlated with disease activity in juvenile-onset lupus.” J Exp Med 206(4): 793-805.
Dressel, R., J. Schindehutte, et al. (2008). “The tumorigenicity of mouse embryonic stem cells and in vitro differentiated neuronal cells is controlled by the recipients' immune response.” PLoS One 3(7): e2622.
Eagle, R. A., I. Jafferji, et al. (2009). “Beyond Stressed Self: Evidence for NKG2D Ligand Expression on Healthy Cells.” Curr Immunol Rev 5(1): 22-34.
Eagle, R. A., J. A. Traherne, et al. (2006). “Regulation of NKG2D ligand gene expression.” Hum Immunol 67(3): 159-69.
Eagle, R. A. and J. Trowsdale (2007). “Promiscuity and the single receptor: NKG2D.” Nat Rev Immunol 7(9): 737-44.
Ehrlich, L. I., K. Ogasawara, et al. (2005). “Engagement of NKG2D by cognate ligand or antibody alone is insufficient to mediate costimulation of human and mouse CD8+ T cells.” J Immunol 174(4): 1922-31.
Eleme, K., S. B. Taner, et al. (2004). “Cell surface organization of stress-inducible proteins ULBP and MICA that stimulate human NK cells and T cells via NKG2D.” J Exp Med 199(7): 1005-10.
Fernandez-Messina, L., O. Ashiru, et al. “Differential mechanisms of shedding of the glycosylphosphatidylinositol (GPI)-anchored NKG2D ligands.” J Biol Chem 285(12): 8543-51.
Fernandez-Morera, J. L., S. Rodriguez-Rodero, et al. (2008). “Soluble MHC class I chain-related protein B serum levels correlate with disease activity in relapsing-remitting multiple sclerosis.” Hum Immunol 69(4-5): 235-40.
Fernandez-Morera, J. L., S. Rodriguez-Rodero, et al. (2008). “Genetic influence of the nonclassical major histocompatibility complex class I molecule MICB in multiple sclerosis susceptibility.” Tissue Antigens 72(1): 54-9.
Gambelunghe, G., A. Brozzetti, et al. (2007). “MICA gene polymorphism in the pathogenesis of type 1 diabetes.” Ann N Y Acad Sci 1110: 92-8.
Gasser, S., S. Orsulic, et al. (2005). “The DNA damage pathway regulates innate immune system ligands of the NKG2D receptor.” Nature 436(7054): 1186-90.
Groh, V., A. Bruhl, et al. (2003). “Stimulation of T cell autoreactivity by anomalous expression of NKG2D and its MIC ligands in rheumatoid arthritis.” Proc Natl Acad Sci USA 100(16): 9452-7.
Houchins, J. P., T. Yabe, et al. (1991). “DNA sequence analysis of NKG2, a family of related cDNA clones encoding type II integral membrane proteins on human natural killer cells.” J Exp Med 173(4): 1017-20.
Hue, S., J. J. Mention, et al. (2004). “A direct role for NKG2D/MICA interaction in villous atrophy during celiac disease.” Immunity 21(3): 367-77.
Ito, T., N. Ito, et al. (2008). “Maintenance of hair follicle immune privilege is linked to prevention of NK cell attack.” J Invest Dermatol 128(5): 1196-206.
Kirsten, H., E. Petit-Teixeira, et al. (2009). “Association of MICA with rheumatoid arthritis independent of known HLA-DRB1 risk alleles in a family-based and a case control study.” Arthritis Res Ther 11(3): R60.
Kondo, M., T. Maruoka, et al. “Comparative genomic analysis of mammalian NKG2D ligand family genes provides insights into their origin and evolution.” Immunogenetics.
Larson, J. H., B. M. Marron, et al. (2006). “Genomic organization and evolution of the ULBP genes in cattle.” BMC Genomics 7: 227.
Li, Y., B. Xia, et al. “MICB0106 gene polymorphism is associated with ulcerative colitis in central China.” Int J Colorectal Dis 25(2): 153-9.
Lopez-Hernandez, R., M. Valdes, et al. “Association analysis of MICA gene polymorphism and MICA-129 dimorphism with inflammatory bowel disease susceptibility in a Spanish population.” Hum Immunol 71(5): 512-4.
McFarland, B. J. and R. K. Strong (2003). “Thermodynamic analysis of degenerate recognition by the NKG2D immunoreceptor: not induced fit but rigid adaptation.” Immunity 19(6): 803-12.
McGilvray, R. W., R. A. Eagle, et al. “ULBP2 and RAET1E NKG2D ligands are independent predictors of poor prognosis in ovarian cancer patients.” Int J. Cancer.
McGilvray, R. W., R. A. Eagle, et al. (2009). “NKG2D ligand expression in human colorectal cancer reveals associations with prognosis and evidence for immunoediting.” Clin Cancer Res 15(22): 6993-7002.
O'Callaghan, C. A., A. Cerwenka, et al. (2001). “Molecular competition for NKG2D: H60 and RAE1 compete unequally for NKG2D with dominance of H60.” Immunity 15(2): 201-11.
Ogasawara, K., J. A. Hamerman, et al. (2004). “NKG2D blockade prevents autoimmune diabetes in NOD mice.” Immunity 20(6): 757-67.
Paschen, A., A. Sucker, et al. (2009). “Differential clinical significance of individual NKG2D ligands in melanoma: soluble ULBP2 as an indicator of poor prognosis superior to S100B.” Clin Cancer Res 15(16): 5208-15.
Rodriguez-Rodero, S., L. Rodrigo, et al. (2006). “MHC class I chain-related gene B promoter polymorphisms and celiac disease.” Hum Immunol 67(3): 208-14.
Stern-Ginossar, N., C. Gur, et al. (2008). “Human microRNAs regulate stress-induced immune responses mediated by the receptor NKG2D.” Nat Immunol 9(9): 1065-73.
Suemizu, H., M. Radosavljevic, et al. (2002). “A basolateral sorting motif in the MICA cytoplasmic tail.” Proc Natl Acad Sci USA 99(5): 2971-6.
Sutherland, C. L., B. Rabinovich, et al. (2006). “ULBPs, human ligands of the NKG2D receptor, stimulate tumor immunity with enhancement by IL-15.” Blood 108(4): 1313-9.
Van Belle, T. L. and M. G. von Herrath (2009). “The role of the activating receptor NKG2D in autoimmunity.” Mol Immunol 47(1): 8-11.
Ward, J., M. Bonaparte, et al. (2007). “HIV modulates the expression of ligands important in triggering natural killer cell cytotoxic responses on infected primary T-cell blasts.” Blood 110(4): 1207-14.
Wilkinson, G. W., P. Tomasec, et al. (2008). “Modulation of natural killer cells by human cytomegalovirus.” J Clin Virol 41(3): 206-12.
Wu, J., Y. Song, et al. (1999). “An activating immunoreceptor complex formed by NKG2D and DAP10.” Science 285(5428): 730-2.
Yabe, T., C. McSherry, et al. (1993). “A multigene family on human chromosome 12 encodes natural killer-cell lectins.” Immunogenetics 37(6): 455-60.

### Example 8

### Alopecia Areata Immunopathogenesis and NKG2D Receptor Ligand Interaction in Mice and Humans

Genomewide Association study undertaken earlier implicates NKG2D receptor-ligand interaction as well as several T-cell specific genes in the immunopathogenesis of alopecia areata (AA). Here the mechanisms of follicular dystrophy mediated by NKG2D+ lymphocytic cytotoxicity against the hair follicle were elucidated. The resident skin and cutaneous lymph node immune population was assessed in C3H/HeJ—murine model of AA and a sizable expansion of the αβ T-cells with immunophenotypic signature of NK reprogramming marked by NKG2D, NKG2A/C/E, CD49b, syk and IL-15 expression was observed. Global transcriptional analysis of AA skin indicated a predominant IFNγ Inflammatory signature. An IFNγ mediated overexpression of NKG2D ligands—ULBP3 and MICA was observed in the HF and HF derived dermal sheath cells ex vivo and in vivo. NKG2D dependent elevated follicular recruitment of lymphocytes and apoptosis is observed after IFNγ treatment and recapitulated in the AA follicle. Interestingly several microRNAs putatively binding to ULBPs was downregulated in skin and was shown to suppress the expression in vitro. Thus gamma interferon plays a vital role in AA etiology by priming the immune system and the end organ for NKG2D mediated cytolysis.

Introduction.

Alopecia Areata (AA) is a widespread autoimmune disorder affecting close to 5 million people in United States and holds a lifetime risk of 1.7% in the general population. The disease etiology comprises an autoimmune attack against the hair follicles (HF) in the skin, infiltration of the surrounding skin with immune-response cells and elevated inflammatory cytokine and chemokine levels resulting in cessation of hair growth and subsequent non scarring alopecia. Interestingly, alopecia areata is often associated with other autoimmune disorders such as celiac disease, rheumatoid arthritis and Type I diabetes.

Hair follicle being a micro-organ represents a special niche where cellular components of mesenchymal, epithelial and neuroectodermal origin interact and sequestration of potentially autoreactive antigens, making the HF susceptible to immune attack as seen in conditions of inflammation such as lichen planopilaris, folliculitis decalvans and autoimmune disorders which initiate hair pathology—Primarily AA, SLE, scleroderma or leukotrichia—(vitiligo). Normally, several mechanisms enable immune tolerance in the hair follicle—the levels of major histocompatibilty family proteins are low—inhibiting detection of reactive autoantigens, release of immunosuppressive cytokines and hormones such as TGFb, ACTH, IGF1 by anagen hair bulb. The number perifollicular as well as intrafollicular lymphocytes and the antigen presenting langerhans cells numbers are low are very low compared to dermis and epidermis (Paus, Nickoloff et al. 2005).

Alopecia areata is characterized by presence of CD8+ve T-cells intrafollicular and CD4+ve T-cells perifollicular infiltrates (Todes-Taylor, Turner et al. 1984). NK cells are also present in the infiltrate (Ito, Ito et al. 2008). In severe cases of alopecia areata greater number of NK and T-cell populations is observed in the peripheral blood lymphocytes of the AA patients (Imai, Miura et al. 1989). Activating receptors present on the surface of immune cells recognize viral associated antigens or aberrant self antigens and trigger cytotoxic activity (Bottino, Castriconi et al. 2005). NKG2D, an activating cell surface receptor is present canonically on the surface of NK, NKT, γδ T-Cells and all human and activated mouse CD8+ve T-cells (Ehrlich, Ogasawara et al. 2005), Interferon producing killer dendritic cells (Chan, Crafton et al. 2006) and regulatory T-cells. NKG2D receptor lacks an intracellular signaling domain and requires the adaptor protein DAP10 for downstream signal transduction via syk and PI3K pathway (Wu, Song et al. 1999). NKG2D receptor shows promiscuous binding to a variety of ligands belonging to the non classical members of the MHC superfamily. Two classes of NKG2D Ligands are present in humans donated as MIC (A and B) and the ULBP (1-6) family and three in mice—Rae1 (α-ε) {retinoic acid early inducible}, H60 {histocompatibility antigen 60} and Mult1 {murine ULBP-like transcript 1}. The Families differ in their structure, chromosomal position and sequence (Eagle and Trowsdale 2007).

NKG2D ligands act as a first line of defense alerting the innate immune system of the presence of aberrant or transformed cells. Stressors which cause cellular damage such as heat shock, oxidative stress or pharmacological agents such as (proteasome inhibitors, HDAC inhibitors—trichostatin A, valproic acids and cisplatin) induce NKG2D expression. (Eagle, Jafferji et al. 2009). The surface expression of NKG2D ligands is also regulated at a transcriptional and post translational level. At a transcriptional level the promoter regions of the ligands contain different putative transcription factor binding sites influencing differential tissue specific expression as well as regulation under stress (Eagle, Traherne et al. 2006). A number of microRNAs have also been shown to bind the 3′UTR of MIC genes and inhibit the transcript levels of the ligands (Stern-Ginossar, Gur et al. 2008).

Cytokine profile of alopecia areata patients displays a bias towards Th1 response (Ghoreishi, Martinka et al.; Barahmani, Lopez et al. 2009) and IFNg levels are elevated in the patient serum and C3H/HeJ mice (Arca, Musabak et al. 2004) (Gilhar, Landau et al. 2003) C3H/HeJ mouse strain, which is genetically susceptible to AA fails to develop lesions when deficient in IFN-γ (Freyschmidt-Paul, McElwee et al. 2006). IFN-γ inducible chemokines MIG, MCP1 and IP-10 are present in AA skin which further sets up a cycle of recruitment of activated T-cells, B-cells, NK and dendritic cells into the tissue (Benoit, Toksoy et al. 2003). Proinflammatory cytokines serum levels—IL-1b, IL-2, IL-12, IL-6 and IL-10 are significantly elevated in patients (Hoffmann 1999; Barahmani, Lopez et al. 2009). This proinflammatory microenvironment of the diseased skin is associated with induction of activating ligands MHC class I and II antigens and Fas ligand on the AA hair follicle (Bodemer, Peuchmaur et al. 2000). MICA and ULBP3—NKG2D ligands are also upregulated in the AA follicle and are a potential recruiter of the cytotoxic T-cells and NK cells. (Ito, Ito et al. 2008), (Petukhova, Duvic et al. 2010)). NKG2D functions to eliminate the aberrant self cells and dysregulation of this recognition process often leads to development of autoimmunity disorders. Interestingly, a study also demonstrated an infiltration of the peribulbular tissue with NKG2D+ve CD8 and NK cells. NKG2D as well as NKG2C density were higher in NK cells of AA patients (Ito, Ito et al. 2008). Previous work showed for the first time the involvement of the ULBP family of NKG2D ligands in the pathogenesis of the autoimmune disease—alopecia areata ((Petukhova, Duvic et al. 2010)).

Given the association of NKG2D ligand, IFNG and SOCS1 loci with human AA, without being bound by theory, aberrant NKG2DL up-regulation and persistent NKG2D activation mediated by elevated gamma interferon signaling in skin, can drive AA pathogenesis. Infiltration of AA skin with NK reprogrammed T-cells which bear NK specific markers such as DX5 and NKG2A/C/E accompanied with elevated expression of inducing interleukin 15 in the hair follicle, as well as surrounding immune cells, was observed. The numbers of NKG2D bearing cytotoxic T-cells were also significantly higher in the cutaneous lymph nodes. Transcriptional profiling of the alopecic skin indicated a massive inflammatory response in the affected skin of the AA mouse model—C3H/HeJ. Further analysis showed a predominant skew towards gamma interferon regulated genes in the AA skin indicating strong interferon signaling in alopecia areata. Hair follicles also exhibited strong NKG2DL expression in response to gamma interferon treatment at both transcriptional as well as translational levels. Preincubation of skin derived primers cells as well as organ cultured HFs with IFNg led to elevated cytotoxicity by lymphokine activated cells. Specific autoimmune mechanisms underlying alopecia areata have remained obscure and given its high prevalence, strong association with other autoimmune disorders and accessibility of HF as disease model warrants a further study of the role of NKG2D receptor-ligand interaction pathway for development of a wide spectrum drug for autoimmune disorders.

Results

NK reprogramming of the T-cells in alopecic skin and cutaneous lymph nodes. NK-Reprogrammed CD8 T Cells infiltrate Alopecia Areata Skin. (a) Immunoflourescence of NKG2D, CD8, CD4 in skin. (b) NKG2D vs. CD8 T cell plot. (c) DX5, NKG2A/C/E, Syk expression of these cells. (d) IL-15 expression in hair follicle.

NKG2D+CD8+ T cells are expanded in alopecic cutaneous lymph nodes. (a) Enlarged cutaneous lymphnodes in AA mice. (b) CD4/CD8-NKG2D/CD8 flow. (c) DX5, CXCR3, CCR5, CD25 flow of these cells. (d) IFN-gamma, IL-17 and Foxp3 of CD4 T cells and CD8 NKG2D positive T cells. (e) and (f) Spectratype and transcriptional profile.

The Interferon gamma response dominates the inflammatory response in AA skin. (a) Interferon producing immune cell types. (b) Heat map for inflammatory/immunegenes. (c) Confirmatory RT-PCRs for microarray. (d) Table of interferon response upregulated genes.

NKG2D ligands are expressed in lesional hair follicles and are upregulated by IFN-g. (a) AA Rae-1 staining in hair follicle. (b) AA upregulated transcripts. (c) Upregulation in situ by injected IFN-gamma. (d) Transcriptional upregulation in vitro-Luciferase assay.

CD8 T cells engage IFN-g primed hair follicles and are cytolytic in an NKG2D-dependent manner. (a) CFSE labeled T cells interact with alopecic but not uninvolved Hair follicles. (b) CFSE labeled T cells interact with IFN-gamma primed hair follicles. (c) Cytotoxic response related gene upregulation in AA. (d) Elevated no. of Apoptotic Cells in DS after cytotoxic killing. (e) Interferon gamma treated dermal sheath cells are sensitized to NKG2D mediated killing.

Human NKG2D-dependent killing assay. (a) Human upregulation of NKG2D ligands. (b) Upregulation when treated with IFNg in DS and fibs. (c) Human cytotoxic cell recruitment. (d) Human NKG2DL overexpression and cytotoxic mediation. (e) Human cytotoxicity assay (repeat for significant p-value).

Stress mediated Micro RNA regulation of NKG2D ligands. (a) Bioinformatics analysis of the 3′UTRs or ULBP3 and ULBP6 for putative microRNA binding sites. (b) RT-PCR for the common microRNA binding sites after IFN, IFN/LPS and TNF treatment. (c) Luciferase assay under stress conditions for IFNg, IFNg/LPS and TNFa in primary cultured cells and 293T cells. (d) Luciferase assay with cotransfected -3′UTR Luciferase construct and microRNA of interest to show there negative effect on mRNA stability.

NK Reprogramming of the T-Cells in Alopecic Skin and Cutaneous Lymph Nodes.

As reported in earlier studies, a predominance of the T-cells in the alopecic skin was observed, as determined by immunofluoroscence staining of the skin by CD8, CD4 T-cells and γδ T-cells. These cells types comprise the main ranks of NKG2D receptor bearing immune population. Co-localization of the CD8 and CD4 T-cells with NKG2D marker was observed in the immune infiltrate surround the hair follicle in the alopecia areata skin. The main cytotoxic T-cell population the NKG2D bearing CD8 cells was analyzed, and it was observed that the cytotoxic T-cells were expanded in the AA skin from (X % to X %) as compared to age matched controls and a greater fraction was NKG2D positive. This phenomenon is reminiscent of NK reprogramming observed in celiac disease a closely related autoimmune (16682498). Thus, the cytotoxic T-cells for other NK specific markers—DX5, NKG2A/C/E and Syk, was further analyzed.

IL-15 levels in the skin of AA compared to age matched were analyzed, and comparatively higher levels in the HF were observed, as well as expression in immune cells comprising the infiltrate. Thus skin comprises of higher levels of NKG2D bearing NK like T-cells.

The cutaneous lymph node immune cell population was further analyzed. Both the axillary and inguinal as well as the spleen were enlarged in the AA mouse. Flowcytometric analysis of the T-cells showed a skewing of the CD4/CD8 ratio from X to X indicating an expansion of cytotoxic phenotype. Greater percentage of the CD positive T-cells also expressed NKG2D receptor in the lymph nodes.

The Interferon Gamma Response Dominates the Inflammatory Response in AA Skin.

T-cells as well as other immune cells—macrophages, dendritic cells as well as neutrophils enriched in AA skin comprise a major source of gamma interferon in the skin. These cells are known to mediate inflammation and related tissue damage. Transcriptional analysis of the Alopecia areata skin in comparison to unaffected age matched skin was carried out using microarray technology (N=3). Total RNA was isolated from whole skin, and hybridized to the Affymetrix Mouse 430 2.0 Genechip. Using Genespring, we obtained 485 transcripts that were significantly (p≦0.05) and differentially regulated (≧2×).

The alopecia areata skin displayed a predominantly elevated inflammatory signature as indicated by fold change heat map. The microarray data was further confirmed using quantitative real-time PCR and a similar trend of elevated inflammatory markers was observed. The differentially expressed gene were further analyzed for overrepresentation of genes of specific biological pathways using software DAVID and striking evidence for the IFN response in AA, in that 16 of the top 20 induced genes, including the chemokines Cxcl9/10/11, were known to be IFN-response genes. This signature is likely due to Ifng since Type I interferons were not induced in AA skin.

Dominance of IFNg response in the AA skin was independently validated by utilizing an interferon signaling and response qPCR array (Stellarray™) assaying X genes. A significant upregulation (p-value<X) was observed in AA skin with X genes showing greater than two fold upregulation. Interestingly, genes including Icos, Tap2 and Ifng were upregulated in alopecic mice and reside within chromosomal regions significantly associated with AA in our GWAS.

Gamma Interferon Mediated NKG2D Ligand Overexpression and Cytotoxicity in Murine and Human Hair Follicle.

NKG2D receptor interfaces with a plethora of NKG2D ligands to mediate its cytolytic effects. The expression of NKG2DLs was further analyzed in AA skin as compared to unaffected a higher expression of all NKG2D ligands as well as expression in HF infiltrate was in AA skin as determined by anti-Rae1 antibodies. Analysis of the transcript levels of different rae 1 isoforms, H60 and multi indicated by a general upregulation of the nkg2d ligand transcripts with significant expression of rae 1e and h60 p<0.05. To examine the situation in vivo, NKG2DL induction was examined in murine skin after intra-dermal injections of IFNγ, LPS and IFNγ/LPS. Staining of the skin, 24 hour post-treatment showed that both IFNγ and TLRs induced total NKG2DL and Rae1 expression in the hair follicles, predicting their sensitivity to NKG2D-mediated cytotoxic attack. To assess the role of inflammatory cytokines on ULBP promoter activity, dermal sheath cells were transfected with luciferase reporter construct containing 3′ upstream 5-kb promoter region of ulbp3 gene. A significant elevation in the promoter activity was observed in ULBP3 following an 8 hr IFNγ treatment (p-value<0.01). Similar increase of ulbp3 promoter activity was observed after 16 hr IFNγ treatment of dermal sheath and fibroblasts.

C3H/HeJ mice vibrissae follicles were microdissected and organ cultured for 2 days in presence of proinflammatory cytokine—IFNγ and TLR ligand—LPS. Individual follicles were subsequently incubated with green CFSE labeled LAK cells (IL-2 stimulated PBMCs) overnight to assess immune interaction. Increased accumulation of LAK cells was observed on treated follicles indicating an up-regulation of interacting ligands. Interestingly, untreated follicles derived from alopecic mice but not unaffected mice also showed enhanced LAK cell recruitment presumably due to NKG2DL upregulation in vivo. Several transcripts associated with cytotoxic immune response category derived from Gene Ontology website (http://www.geneontology.org/) were upregulated in the AA skin as compared to age matched controls. It was further determined whether increased immune recruitment to the hair follicle is associated with higher apoptosis in the dermal sheath layer. Indeed, a higher percentage of TUNEL positive cells in the IFNγ and LPS treated follicles was observed, as compared to the untreated.

A lactate dehydrogenase release based cytotoxicity assay was established, using primary cultured dermal sheath or dermal papilla cells as target cell population and splenocytes expanded for 7 days in high dose IL-2 as cytotoxic effectors. CD8 T-cells from these cultures, so-called “lymphokine activated killer” or LAK cells, express NKG2D. Consistent with prior data demonstrating NKG2DL induction, IFNγ and LPS treatment for 3 days rendered DS cells sensitive to LAK-mediated cytotoxicity in an NKG2D-dependent manner.

Human hair follicles were micro-dissected and organ cultured for 2 days in the presence of IFNγ with or without TLR ligands. Immunofluorescence staining of the human follicles for NKG2D Ligands—MICA, ULBP3 and Pan NKG2DL shows higher expression in the DS compartment of the hair follicle post treatment. To examine whether IFN-γ directly regulates NKG2DL transcription, dermal sheath (DS) cells were derived and primary cultured from micro-dissected human hair follicles and treated with IFNγ for 24 h and the transcript levels of NKG2DLs were assessed by real-time qPCR (N=4). Message levels of NKG2DLs ULBP3 and MICA were upregulated. The protein expression induction by IFN-γ is stronger than that seen at the RNA level for NKG2D Ligands, indicating pos-transcriptional regulation. Organ cultured scalp derived human HFs in presence of proinflammatory cytokine—IFNγ and TLR ligand—LPS were incubation with LAK (lymphokine activated killer) cells. Treated HFs yielded greater lymphocytic recruitment to the follicular surface upon LAK coincubation. The specificity of this interaction was further tested using lactate dehydrogenase release based cytotoxicity assay using cultured skin derived epithelial (keratinocytes) cells. Keratinocyte lysis by LAK cells was blocked by anti-NKG2D or MHC-1 antibodies, thus confirming the dependence of cytotoxicity on these signals.

Interferon Dependent Regulation of NKG2DL Expression by microRNAs. (a)

Bioinformatics analysis of the 3′UTRs or ULBP3 and ULBP6 for putative microRNA binding sites; (b) RT-PCR for the common microRNA binding sites after IFN, IFN/LPS and TNF treatment; (c) Luciferase assay under stress conditions for IFNg, IFNg/LPS and TNFa in primary cultured cells and 293T cells. (d) Luciferase assay with cotransfected -3′UTR Luciferase construct and microRNA of interest to show there negative effect on mRNA stability (e.g., mir124).

Discussion

A paradigm shifting model to explain the emergence of autoimmunity was proposed by Polly Matzinger which postulates that immune system reacts in response to danger signals presented by damaged or distressed tissue and autoimmunity arises when the danger signals do not resolve and are presented chronically (Matzinger 2002). Thus autoimmunity is inherent but transient in normal individuals but acquires pathology when activated long term. In the model's context, danger signals are defined as intrinsic cellular components which are released or presented by cells under conditions of stress, damage or inappropriate cell death (necrosis). Various cellular components have been identified as danger signals or “alarmins”—HMGB1, S100s, heatshock proteins, uric acid etc (Tveita) (Bianchi 2007). Several scenarios can lead to development of autoimmunity under this model. Highly specialized organ specific antigens normally sequestered within the cell, when aberrantly displayed on antigen presenting cells (APCs) can act as danger signals. This is observed in case of vitiligo and alopecia areata where anti-melanocytic autoantibodies are presented. The development of autoimmunity is decided by whether or not tolerogenic signals prevail over immunogenic or activating signals. In alopecia areata the tolerogenic signals diminish as the MHCI levels increase on hair follicles combined with increase in the activation signaling to cytotoxic cells by NKG2D ligands MICA and ULBPs. APCs play an important role as a switch between tolerance and immunogenicity.

NKG2D functions to eliminate the aberrant self cells and dysregulation of this recognition process often leads to development of autoimmunity disorders (Van Belle and von Herrath 2009). In rheumatoid arthritis patients, greater numbers of circulating as well as resident CD4 positive cells express NKG2D ligand. These Helper T-cells exhibit a cytotoxic profile with secretion of IFNg, perforin, granzyme B and cytolytic ability. The synoviocytes in RA also secrete soluble MICA into the synovial fluid. (Groh, Bruhl et al. 2003). Crohn's disease patients exhibit elevated MICA staining in the lamina propria as well as a CD4 positive cells which express NKG2D receptor, secrete IFNg and perforin and are cytolytic (Allez, Tieng et al. 2007). MICA levels were found to be upregulated in active cases of celiac disease which lower with gluten free diet, along with higher soluble MICA concentrations in the patient's sera. Elevated NKG2D density was observed on intraepithelial lymphocytes of patients along with more efficient NKG2D facilitated cytotoxic response against epithelial cells (Hue, Mention et al. 2004).

Non Obese diabetic mice are used as a model of type I diabetes in humans. A study done in these mice elucidates the importance of NKG2D receptor engagement in the development of pancreatic β-cell autoimmunity. The levels of Rae1—the murine NKG2D ligand were elevated in NOD mice compared to control balb/c mice and exhibited progressive increase with age in NOD as well as NOD SCID mice indicating that elevation of rae 1 is independent of immune response. Interestingly, NKG2D neutralizing antibody treatment in NOD mice prevented the development of T1D, underscoring the importance of NKG2D pathway in the development of autoimmunity (Ogasawara, Hamerman et al. 2004). In cases of multiple sclerosis as well elevated MICB serum levels were associated with disease relapse (Fernandez-Morera, Rodriguez-Rodero et al. 2008). Interestingly, a previous study also demonstrated an elevation of MICA ligand in the hair follicle of alopecia areata along with infiltration of the peribulbular tissue with NKG2D+ve CD8 and NK cells. NKG2D as well as NKG2C density were higher in NK cell of AA patients (Ito, Ito et al. 2008) and the data herein).

The involvement of ULBP family of NKG2D ligands in the pathogenesis of the autoimmune disease—alopecia areata was shown for the first time (the GWAS Data). Allelic polymorphisms in NKG2D ligands are increasingly being associated with various autoimmune disorders. Specific MICA alleles are overrepresented in rheumatoid arthritis, inflammatory bowel disease and T1D diabetes patients implicating their role in disease pathogenesis (Kirsten, Petit-Teixeira et al. 2009), (Lopez-Hernandez, Valdes et al.) (Gambelunghe, Brozzetti et al. 2007). MICB polymorphisms are also associated with celiac disease, ulcerative colitis and multiple sclerosis (Li, Xia et al.), (Fernandez-Morera, Rodriguez-Rodero et al. 2008), (Rodriguez-Rodero, Rodrigo et al. 2006).

C3H/HeJ Mice strain of mice presents a spontaneous development of disease in 20% of the population by the age of 18 months (Sundberg, Cordy et al. 1994). AA can be induced in normal C3H/HeJ mice at higher frequencies and in a more predictable manner by full thickness grafting of lesional skin (McElwee, Boggess et al. 1998). Human Skin Grafted Severe combined immune deficient (SCID) mice which lack functional B-cells and T-cells are frequently used to model a human equivalent model of AA. The ability of SCID mice to tolerate xenografts is utilized to graft human skin on mice, which can then be tested by adoptive transfer of AA patient lymphocytes for disease development and remission. (Gilhar, Landau et al. 2002). Both IFN-gamma and FasL are required, consistent with CD8 mediated toxicity driven by Th1 help. (Freyschmidt-Paul, Zoller et al. 2005; McElwee, Freyschmidt-Paul et al. 2005). However this understanding remains incomplete; the cellular sources of IFNgamma/FasL (Freyschmidt-Paul, McElwee et al. 2003; Freyschmidt-Paul, McElwee et al. 2006) are unknown and the specific mechanistic contributions of IFNs have not been described. In particular the contributions of the NKG2D pathway remain unexplored and the GWAS indicate an alternative theory, namely that NKG2D-bearing cells are likely crucial to the innate and subsequent adaptive response.

Materials and Methods

Animals. C3H/HeJ, C57B1/6 and Syk−/− mice at various stages of hair cycle as well as retired breeders were purchased from Jackson Laboratories. The mice were housed in a pathogen free barrier facility. Synchronized anagen was induced in the hair coat by shaving or by plucking. Animals were administered X IFNγ, X LPS and X TNFα and sterile PBS via intradermal injections. Blood was obtained by retro-orbital bleeding and stored in heparinized tubes to prevent coagulation. For tissue harvesting, the skin was shaven, flash frozen in liquid nitrogen and stored at −70° C.

Immune Cell Isolation and Culture from Skin and Cutaneous Lymph Nodes

Ex Vivo Organ Culture.

Scalp biopsies were acquired from clinic. The scalp skin was further microdissected to isolate individual hair follicular units. The HFs were cultured in serum free HF organ culture medium as described in protocols from Kondo and Philpott et al (Philpott, Sanders et al. 1996). Vibrissae hair follicles were also microdissected from murine facepads of C57B1/6 and C3H/HeJ mice and similarly cultured ex vivo for 7-10 days normal anagen growth. Individual follicles were cultured in the presence of 100 ng/ml IFNγ (PeproTech #315-05 or #300-02) individually or in combination with 1 ug/ml of LPS or 1 ug/ml of polydI:dC for 3 days. The follicles were embedded in OCT (Sakura Finetek) and 7-8 um longitudinal sections were cut and stored at −80° C.

Immunohistochemistry.

Mouse skin from age matched and alopecic mice was shaved and fixed in 10% formalin in PBS overnight followed by transfer to 70% ethanol for paraffin embedding. Skin was also embedded in OCT and frozen on dry ice. The frozen blocks were sectioned to a thickness of 7-8 μm. Frozen skin sections or hair follicle cross-sections were air dried and fixed in either 4% paraformaldehyde or Methanol/Acetone (1:1) solution followed by block in 10% normal donkey serum. The sections were incubated with the following antibodies—IL-15( ), Anti Rae1 ( ), ULBP3, MICA, Pan NKG2D Ligand ( ), NKG2D ( ), CD8, CD4, overnight. Following brief wash the sections were incubated with fluorescence labeled secondary antibodies (Invitrogen) and counterstained with DAPI. The sections were further visualized using Axioplan2 fluorescence and LSM5 exciter confocal microscopes from Zeiss. Axiovision and Zen softwares (Zeiss) were further used for image capture and analysis.

Primary Dermal Sheath/Fibroblast Culture.

Human foreskin was used to establish primary cultures of fibroblasts and keratinocytes. Interfollicular skin was dispase treated to separate the epidermal and dermal components and enzymatically processed to establish primary cultures of fibroblasts and keratinocytes. The hair follicles derived from scalp biopsies will be microdissected to separate the dermal sheath and the papilla and further used to culture dermal sheath cells (DS) and dermal papilla cells (DP) from explants.

Over Expression Constructs and Luciferase Assays.

3 kb upstream promoter region of MICA, ULBP3 and ULBP6 were PCR amplified using primers. The fragments were then cloned into pGL3 basic vector plasmid upstream of the Luciferase gene. Dermal Sheath cells and HEK 293T cells were transiently transfected with the luciferase constructs and well as β-gal expression plasmid (e.g., using lipofectamine). 6-8 hours after transfection the 100 ng/ml of IFNγ was added to the media. The cells were harvested 8 hrs after IFNγ treatment and lysates were used to assay Luciferase activity (Promega E4530) on a luminometer. The β-galactosidase activity was assessed using enzymatic colorometric assay (Promega E2000) and read at 415 nm absorbance on a microplate reader after 30 min incubation at 37° C.

Cytotoxicity Assays.

Spleen and lymph nodes were harvested from mice, mashed and passed through 30 and 70 micron filters to obtain single cell immune cell suspension. RBC lysis was carried out to obtain lymphocytic population. The cells were cultured in IL-2 supplemented RPMI medium for a week to derive lymphokine activated killer cells. Organ culture of vibrissae hair follicles was carried out in presence of IFNγ, IFNγ/LPS for 3 days. Subsequently the LAK cells stained with CFSE were incubated with the hair follicle overnight. LAK cell interaction with the HF was visualized under GFP filter in a microscope. Hair follicle were further embedded in OCT and sectioned. TUNEL staining was carried out to determine the number of apoptotic cells. The number of cells was counted. Two tailed T-test was carried out to determine the difference between treatments.

FACS Analysis According to Methods Practiced in the Art.

Real Time-PCR+RT-PCR Arrays.

Total RNA was extracted from frozen livers using the RNeasy purification kit (Qiagen) in accordance with the manufacturer's protocol. DNase-treated total liver RNA was reverse transcribed using SuperScript II reverse transcriptase (Invitrogen). Real time PCR (RT PCR) was performed using SYBR Green Master Mix and the ABI Prism 7000 Sequence Detection System (Applied Biosystems). GAPDH and β-actin were used as internal control genes. Thermal cycling conditions consisted of an initial step at 95° C. for 10 min to activate the Taq DNA polymerase and 40 cycles of sequential denaturation at 95° C. for 15 s and annealing/extension at 60° C. for 60s. Data analysis was performed using the ABI Prism 7000 SDS Software (Applied Biosystems). The real-time PCR analysis was performed according to the comparative CT method (Amador-Noguez, Yagi et al. 2004). The p-values reported for these changes refer to a two-tailed t-test between the normalized CT values. Mouse Interferon Signaling & Response 96 StellARray™ qPCR array (Lonza #00188171) was used for quantitative Realtime PCR analysis of interferon regulated transcripts.

Microarray Data Analysis According to Methods Practiced in the Art.

### REFERENCES

Allez, M., V. Tieng, et al. (2007). “CD4+NKG2D+ T cells in Crohn's disease mediate inflammatory and cytotoxic responses through MICA interactions.” Gastroenterology 132(7): 2346-58.
Amador-Noguez, D., K. Yagi, et al. (2004). “Gene expression profile of long-lived Ames dwarf mice and Little mice.” Aging Cell 3(6): 423-41.
Arca, E., U. Musabak, et al. (2004). “Interferon-gamma in alopecia areata.” Eur J Dermatol 14(1): 33-6.
Barahmani, N., A. Lopez, et al. (2009). “Serum T helper 1 cytokine levels are greater in patients with alopecia areata regardless of severity or atopy.” Clin Exp Dermatol.
Benoit, S., A. Toksoy, et al. (2003). “Selective expression of chemokine monokine induced by interferon-gamma in alopecia areata.” J Invest Dermatol 121(4): 933-5.
Bianchi, M. E. (2007). “DAMPs, PAMPs and alarmins: all we need to know about danger.” J Leukoc Biol 81(1): 1-5.
Bodemer, C., M. Peuchmaur, et al. (2000). “Role of cytotoxic T cells in chronic alopecia areata.” J Invest Dermatol 114(1): 112-6.
Bottino, C., R. Castriconi, et al. (2005). “Cellular ligands of activating NK receptors.” Trends Immunol 26(4): 221-6.
Chan, C. W., E. Crafton, et al. (2006). “Interferon-producing killer dendritic cells provide a link between innate and adaptive immunity.” Nat Med 12(2): 207-13.
Eagle, R. A., I. Jafferji, et al. (2009). “Beyond Stressed Self: Evidence for NKG2D Ligand Expression on Healthy Cells.” Curr Immunol Rev 5(1): 22-34.
Eagle, R. A., I. A. Traherne, et al. (2006). “Regulation of NKG2D ligand gene expression.” Hum Immunol 67(3): 159-69.
Eagle, R. A. and J. Trowsdale (2007). “Promiscuity and the single receptor: NKG2D.” Nat Rev Immunol 7(9): 737-44.
Ehrlich, L. I., K. Ogasawara, et al. (2005). “Engagement of NKG2D by cognate ligand or antibody alone is insufficient to mediate costimulation of human and mouse CD8+ T cells.” J Immunol 174(4): 1922-31.
Fernandez-Morera, J. L., S. Rodriguez-Rodero, et al. (2008). “Soluble MHC class I chain-related protein B serum levels correlate with disease activity in relapsing-remitting multiple sclerosis.” Hum Immunol 69(4-5): 235-40.
Fernandez-Morera, J. L., S. Rodriguez-Rodero, et al. (2008). “Genetic influence of the nonclassical major histocompatibility complex class I molecule MICB in multiple sclerosis susceptibility.” Tissue Antigens 72(1): 54-9.
Freyschmidt-Paul, P., K. J. McElwee, et al. (2003). “Fas-deficient C3.MRL-Tnfrsf6(lpr) mice and Fas ligand-deficient C3H/HeJ-Tnfsf6(gld) mice are relatively resistant to the induction of alopecia areata by grafting of alopecia areata-affected skin from C3H/HeJ mice.” J Investig Dermatol Symp Proc 8(1): 104-8.
Freyschmidt-Paul, P., K. J. McElwee, et al. (2006). “Interferon-gamma-deficient mice are resistant to the development of alopecia areata.” Br J Dermatol 155(3): 515-21.
Freyschmidt-Paul, P., M. Zoller, et al. (2005). “The functional relevance of the type 1 cytokines IFN-gamma and IL-2 in alopecia areata of C3H/HeJ mice.” J Investig Dermatol Symp Proc 10(3): 282-3.
Gambelunghe, G., A. Brozzetti, et al. (2007). “MICA gene polymorphism in the pathogenesis of type 1 diabetes.” Ann N Y Acad Sci 1110: 92-8.
Ghoreishi, M., M. Martinka, et al. “Type 1 interferon signature in the scalp lesions of alopecia areata.” Br J. Dermatol.
Gilhar, A., M. Landau, et al. (2002). “Mediation of alopecia areata by cooperation between CD4+ and CD8+ T lymphocytes: transfer to human scalp explants on Prkdc (scid) mice.” Arch Dermatol 138(7): 916-22.
Gilhar, A., M. Landau, et al. (2003). “Transfer of alopecia areata in the human scalp graft/Prkdc (scid) (SCID) mouse system is characterized by a TH1 response.” Clin Immunol 106(3): 181-7.
Groh, V., A. Bruhl, et al. (2003). “Stimulation of T cell autoreactivity by anomalous expression of NKG2D and its MIC ligands in rheumatoid arthritis.” Proc Natl Acad Sci USA 100(16): 9452-7.
Hoffmann, R. (1999). “The potential role of cytokines and T cells in alopecia areata.” J Investig Dermatol Symp Proc 4(3): 235-8.
Hue, S., J. J. Mention, et al. (2004). “A direct role for NKG2D/MICA interaction in villous atrophy during celiac disease.” Immunity 21(3): 367-77.
Imai, R., J. Miura, et al. (1989). “[Correlation of activated T cells and natural killer (NK) cells with the disease activity of alopecia areata (AA)].” Nippon Hifuka Gakkai Zasshi 99(6): 743-5.
Ito, T., N. Ito, et al. (2008). “Maintenance of hair follicle immune privilege is linked to prevention of NK cell attack.” J Invest Dermatol 128(5): 1196-206.
Kirsten, H., E. Petit-Teixeira, et al. (2009). “Association of MICA with rheumatoid arthritis independent of known HLA-DRB1 risk alleles in a family-based and a case control study.” Arthritis Res Ther 11(3): R60.
Li, Y., B. Xia, et al. “MICB0106 gene polymorphism is associated with ulcerative colitis in central China.” Int J Colorectal Dis 25(2): 153-9.
Lopez-Hernandez, R., M. Valdes, et al. “Association analysis of MICA gene polymorphism and MICA-129 dimorphism with inflammatory bowel disease susceptibility in a Spanish population.” Hum Immunol 71(5): 512-4.
Matzinger, P. (2002). “The danger model: a renewed sense of self.” Science 296(5566): 301-5.
McElwee, K. J., D. Boggess, et al. (1998). “Experimental induction of alopecia areata-like hair loss in C3H/HeJ mice using full-thickness skin grafts.” J Invest Dermatol 111(5): 797-803.
McElwee, K. J., P. Freyschmidt-Paul, et al. (2005). “Transfer of CD8(+) cells induces localized hair loss whereas CD4(+)/CD25(−) cells promote systemic alopecia areata and CD4(+)/CD25(+) cells blockade disease onset in the C3H/HeJ mouse model.” J Invest Dermatol 124(5): 947-57.
Ogasawara, K., J. A. Hamerman, et al. (2004). “NKG2D blockade prevents autoimmune diabetes in NOD mice.” Immunity 20(6): 757-67.
Paus, R., B. J. Nickoloff, et al. (2005). “A ‘hairy’ privilege.” Trends Immunol 26(1): 32-40.
Petukhova, L., M. Duvic, et al. (2010). “Genome-wide association study in alopecia areata implicates both innate and adaptive immunity.” Nature 466(7302): 113-7.
Philpott, M. P., D. A. Sanders, et al. (1996). “Whole hair follicle culture.” Dermatol Clin 14(4): 595-607.
Rodriguez-Rodero, S., L. Rodrigo, et al. (2006). “MHC class I chain-related gene B promoter polymorphisms and celiac disease.” Hum Immunol 67(3): 208-14.
Stern-Ginossar, N., C. Gur, et al. (2008). “Human microRNAs regulate stress-induced immune responses mediated by the receptor NKG2D.” Nat Immunol 9(9): 1065-73.
Sundberg, J. P., W. R. Cordy, et al. (1994). “Alopecia areata in aging C3H/HeJ mice.” J Invest Dermatol 102(6): 847-56.
Todes-Taylor, N., R. Turner, et al. (1984). “T cell subpopulations in alopecia areata.” J Am Acad Dermatol 11(2 Pt 1): 216-23.
Tveita, A. A. “The danger model in deciphering autoimmunity.” Rheumatology (Oxford) 49(4): 632-9.
Van Belle, T. L. and M. G. von Herrath (2009). “The role of the activating receptor NKG2D in autoimmunity.” Mol Immunol 47(1): 8-11.
Wu, J., Y. Song, et al. (1999). “An activating immunoreceptor complex formed by NKG2D and DAP10.” Science 285(5428): 730-2.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A method for detecting the presence of or a predisposition to a hair-loss disorder in a human subject, the method comprising:
(a) obtaining a biological sample from a human subject; and
(b) detecting whether or not there is an alteration in the level of expression of an mRNA or a protein encoded by a HLDGC gene in the subject as compared to the level of expression in a subject not afflicted with a hair-loss disorder.

2. A method for detecting the presence of or a predisposition to a hair-loss disorder in a human subject, the method comprising:
(a) obtaining a biological sample from a human subject; and
(b) detecting the presence of one or more nucleotide polymorphisms (SNPs) in a chromosome region containing a HLDGC gene in the subject, wherein the SNP is selected from the SNPs listed in Table 2.

3. The method of claim 1, wherein the detecting comprises determining whether mRNA expression or protein expression of the HLDGC gene is increased or decreased as compared to expression in a normal sample.

4. The method of claim 1, wherein the detecting comprises determining in the sample whether expression of at least 2 HLDGC proteins, at least 3 HLDGC proteins, at least 4 HLDGC proteins, at least 5 HLDGC proteins, at least 6 HLDGC proteins, at least 6 HLDGC proteins, at least 7 HLDGC proteins, or at least 8 HLDGC proteins is increased or decreased as compared to expression in a normal sample.

5. The method of claim 1, wherein the detecting comprises determining in the sample whether expression of at least 2 HLDGC mRNAs, at least 3 HLDGC mRNAs, at least 4 HLDGC mRNAs, at least 5 HLDGC mRNAs, at least 6 HLDGC mRNAs, at least 6 HLDGC mRNAs, at least 7 HLDGC mRNAs, or at least 8 HLDGC mRNAs is increased or decreased as compared to expression in a normal sample.

6. The method of claim 2, wherein the chromosome region comprises region 2q33.2, region 4q27, region 4q31.3, region 5p13.1, region 6q25.1, region 9q31.1, region 10p15.1, region 11q13, region 12q13, region 6p21.32, or a combination thereof.

7. The method of claim 1, or 2, wherein the detecting comprises gene sequencing, selective hybridization, selective amplification, gene expression analysis, or a combination thereof.

8. The method of claim 3, wherein an increase in the expression of at least 2 HLDGC genes, at least 3 HLDGC genes, at least 4 HLDGC genes, at least 5 HLDGC genes, at least 6 HLDGC genes, at least 7 HLDGC genes, or at least 8 HLDGC genes indicates a predisposition to or presence of a hair-loss disorder in the subject.

9. The method of claim 3, wherein a decrease in the expression of at least 2 HLDGC genes, at least 3 HLDGC genes, at least 4 HLDGC genes, at least 5 HLDGC genes, at least 6 HLDGC genes, at least 7 HLDGC genes, or at least 8 HLDGC genes indicates a predisposition to or presence of a hair-loss disorder in the subject.

10. The method of claim 3, wherein the mRNA or protein expression level of the HLDGC gene in the subject is about 5-fold to about 70-fold increased, as compared to that in the normal sample.

11. The method of claim 3, wherein the mRNA or protein expression level of the HLDGC gene in the subject is about 5-fold to about 90-fold increased, as compared to that in the normal sample.

12. The method of claim 3, wherein the mRNA or protein expression level of the HLDGC gene in the subject is about 5-fold to about 70-fold decreased, as compared to that in the normal sample.

13. The method of claim 3, wherein the mRNA or protein expression level of the HLDGC gene in the subject is about 5-fold to about 90-fold decreased, as compared to that in the normal sample.

14. The method of claim 1, wherein the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2.

15. The method of claim 14, wherein the HLA Region residing gene is selected from the group consisting of a gene of the HLA Class I Region, a gene of the HLA Class II Region, PTPN22, and AIRE.

16. The method of claim 15, wherein the HLA Class I Region gene is HLA-A, HLA-B, HLA-C, HLA-DQB1, HLA-DRB1, MICA, MICB, HLA-G, or NOTCH4.

17. The method of claim 16, wherein the HLA Class II Region gene is HLA-DOB, HLA-DQA1, HLA-DQA2, HLA-DQB2, TAP2, or HLA-DRA.

18. The method of claim 1 or 2, wherein the hair-loss disorder comprises androgenetic alopecia, alopecia areata, telogen effluvium, alopecia totalis, hypotrichosis, hereditary hypotrichosis simplex, or alopecia universalis.

19. The method of claim 2, wherein the single nucleotide polymorphism is selected from the group consisting of rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, and rs6910071 rs6910071 (SEQ ID NOS 6153-6170, respectively, in order of appearance).

20. A cDNA- or oligonucleotide-microarray for diagnosis of a hair-loss disorder, wherein the microarray comprises SEQ ID NOS: 2, 4, 6, 8, 10, 12, 14, 16, 18, 20, 22, 24, or a combination thereof.

21. A cDNA- or oligonucleotide-microarray for diagnosis of a hair-loss disorder, wherein the microarray comprises SNPs listed in Table 2.

22. A cDNA- or oligonucleotide-microarray for diagnosis of a hair-loss disorder, wherein the microarray comprises SNPs rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, rs6910071, or a combination thereof (SEQ ID NOS 6153-6170, respectively, in order of appearance).

23. A method for determining whether a subject exhibits a predisposition to a hair-loss disorder using the microarray of claim 20, 21, or 22, the method comprising:
(a) obtaining a nucleic acid sample from the subject;
(b) performing a hybridization to form a double-stranded nucleic acid between the nucleic acid sample and a probe; and
(c) detecting the hybridization.

24. The method of claim 23, wherein the hybridization is detected radioactively, by fluorescence, or electrically.

25. The method of claim 23, wherein the nucleic acid sample comprises DNA or RNA.

26. The method of claim 23, wherein the nucleic acid sample is amplified.

27. A diagnostic kit for determining whether a sample from a subject exhibits a predisposition to a hair-loss disorder, the kit comprising a cDNA- or oligonucleotide-microarray of claim 20, 21, or 22.

28. A diagnostic kit for determining whether a sample from a subject exhibits increased or decreased expression of at least 2 or more HLDGC genes, the kit comprising a nucleic acid primer that specifically hybridizes to one or more HLDGC genes.

29. A diagnostic kit for determining whether a sample from a subject exhibits a predisposition to a hair-loss disorder, the kit comprising a nucleic acid primer that specifically hybridizes to a single nucleotide polymorphism (SNP) in a chromosome region containing a HLDGC gene, wherein the primer will prime a polymerase reaction only when a SNP of Table 2 is present.

30. The kit of claim 28 or 29, wherein the primer comprises a nucleotide sequence selected from the group consisting of SEQ ID NOS: 25-40 in Table 9.

31. The kit of claim 29, wherein the SNP is selected from the group consisting of rs1024161, rs3096851, rs7682241, rs361147, rs10053502, rs9479482, rs2009345, rs10760706, rs4147359, rs3118470, rs694739, rs1701704, rs705708, rs9275572, rs16898264, rs3130320, rs3763312, and rs6910071 (SEQ ID NOS 6153-6170, respectively, in order of appearance).

32. The kit of claim 28 or 29, wherein the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2.

33. The kit of claim 32, wherein the HLA Region residing gene is selected from the group consisting of a gene of the HLA Class I Region, a gene of the HLA Class II Region, PTPN22, and AIRE.

34. The kit of claim 33, wherein the HLA Class I Region gene is HLA-A, HLA-B, HLA-C, HLA-DQB1, HLA-DRB1, MICA, MICB, HLA-G, or NOTCH4.

35. The kit of claim 33, wherein the HLA Class II Region gene is HLA-DOB, HLA-DQA1, HLA-DQA2, HLA-DQB2, TAP2, or HLA-DRA.

36. A composition for modulating HLDGC protein expression or activity in a subject wherein the composition comprises an antibody that specifically binds to a HLDGC protein or a fragment thereof; an antisense RNA that specifically inhibits expression of a HLDGC gene that encodes the HLDGC protein; or a siRNA that specifically targets a HLDGC gene encoding the HLDGC protein.

37. The composition of claim 36, wherein the siRNA comprises a nucleic acid sequence comprising any one sequence of SEQ ID NOS: 41-6152.

38. The composition of claim 36, wherein the siRNA is directed to ULBP3, ULBP6, or PRDX5.

39. The composition of claim 36, wherein the antibody is directed to ULBP3, ULBP6, or PRDX5.

40. A method for inducing hair growth in a subject, the method comprising:
(a) administering to the subject an effective amount of a HLDGC modulating compound,
thereby controlling hair growth in the subject.

41. The method of claim 40, wherein the HLDGC gene is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2.

42. The method of claim 41, wherein the HLA Region residing gene is selected from the group consisting of a gene of the HLA Class I Region, a gene of the HLA Class II Region, PTPN22, and AIRE.

43. The method of claim 42, wherein the HLA Class I Region gene is HLA-A, HLA-B, HLA-C, HLA-DQB1, HLA-DRB1, MICA, MICB, HLA-G, and NOTCH4.

44. The method of claim 42, wherein the HLA Class II Region gene is HLA-DOB, HLA-DQA1, HLA-DQA2, HLA-DQB2, TAP2, and HLA-DRA.

45. The method of claim 40, wherein the modulating compound comprises an antibody that specifically binds to a the HLDGC protein or a fragment thereof; an antisense RNA that specifically inhibits expression of a HLDGC gene that encodes the HLDGC protein; or a siRNA that specifically targets the HLDGC gene encoding the HLDGC protein.

46. The method of claim 40, wherein the subject is afflicted with a hair-loss disorder.

47. The method of claim 46, wherein the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis.

48. A method for identifying a compound useful for treating alopecia areata or an immune disorder, the method comprising:
(a) contacting a NKG2D-positive (+) cell with a test agent in vitro in the presence of a NKG2D ligand; and
(b) determining whether the test agent altered the cell response to the ligand binding to the NKG2D receptor as compared to an NKG2D+ cell contacted with the NKG2D ligand in the absence of the test agent,
thereby identifying a compound useful for treating alopecia areata or an immune disorder.

49. The method of claim 48, wherein the test agent specifically binds a NKG2D ligand.

50. The method of claim 48, wherein the NKG2D ligand comprises ULBP1, ULBP2, ULBP3, ULBP4, ULBP5, ULBP6, or a combination thereof.

51. The method of claim 48, wherein the determining comprises measuring ligand-induced NKG2D activation of the NKG2D+ cell.

52. The method of claim 48, wherein the compound decreases downstream receptor signaling of the NKG2D protein.

53. The method of claim 48, wherein measuring ligand-induced NKG2D activation comprises one or more of measuring NKG2D internalization, DAP10 phosphorylation, p85 PI3 kinase activity, Akt kinase activity, production of IFNγ, and cytolysis of a NKG2D-ligand+ target cell.

54. The method of claim 48, wherein the NKG2D+ cell is a lymphocyte or a hair follicle cell.

55. The method of claim 54, wherein the lymphocyte is a Natural Killer cell, γδ-TcR+ T cell, CD8+ T cell, a CD4+ T cell, or a B cell.

56. A method of treating a hair-loss disorder in a mammalian subject in need thereof, the method comprising administering to the subject an antibody or antibody fragment that binds ULBP3, ULBP6, or PRDX5.

57. A method of treating a hair-loss disorder in a mammalian subject in need thereof, the method comprising administering to the subject an RNA molecule that specifically targets the ULBP3 gene encoding the ULBP3 protein.

58. A method of treating a hair-loss disorder in a mammalian subject in need thereof, the method comprising administering to the subject an RNA molecule that specifically targets the ULBP6 gene encoding the ULBP6 protein.

59. A method of treating a hair-loss disorder in a mammalian subject in need thereof, the method comprising administering to the subject an RNA molecule that specifically targets the PRDX5 gene encoding the PRDX5 protein.

60. The method of claim 57, 58, or 59, wherein the RNA molecule is an antisense RNA or a siRNA.

61. A method for treating or preventing a hair-loss disorder in a mammalian subject in need thereof, the method comprising administering to the subject a therapeutic amount of a pharmaceutical composition comprising a functional HLDGC gene that encodes the HLDGC protein, or a functional HLDGC protein, thereby treating or preventing a hair-loss disorder.

62. A method for treating or preventing a hair-loss disorder in a mammalian subject in need thereof, the method comprising administering to the subject a therapeutic amount of a pharmaceutical composition comprising the composition of claim 36, thereby treating or preventing a hair-loss disorder.

63. The method of claim 56, 57, 58, 59, 61, or 62, wherein the administering comprises a subcutaneous, intra-muscular, intra-peritoneal, or intravenous injection; an infusion; oral, nasal, or topical delivery; or a combination thereof.

64. The method of claim 61, wherein the administering comprises delivery of a functional HLDGC gene that encodes the HLDGC protein, or a functional HLDGC protein to the epidermis or dermis of the subject.

65. The method of claim 62, wherein the administering comprises delivery of the composition to the epidermis or dermis of the subject.

66. The method of claim 56, 57, 58, 59, 61, or 62, wherein administering occurs daily, weekly, twice weekly, monthly, twice monthly, or yearly.

67. The method of claim 61, wherein the HLDGC gene or protein is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2.

68. The method of claim 67, wherein the HLA Region residing gene is selected from the group consisting of a gene of the HLA Class I Region, a gene of the HLA Class II Region, PTPN22, and AIRE.

69. The method of claim 68, wherein the HLA Class I Region gene is HLA-A, HLA-B, HLA-C, HLA-DQB1, HLA-DRB1, MICA, MICB, HLA-G, and NOTCH4.

70. The method of claim 68, wherein the HLA Class II Region gene is HLA-DOB, HLA-DQA1, HLA-DQA2, HLA-DQB2, TAP2, and HLA-DRA.

71. The method of claim 56, 57, 58, 59, 61, or 62, wherein the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis.

72. The method of claim 40, wherein the modulating compound comprises a fusion protein that specifically binds to a HLDGC protein or a fragment thereof.

73. The method of claim 72, wherein the fusion protein is directed to CTLA-4.

74. The method of claim 72, wherein the fusion protein is CTLA4-Ig.

75. The method of claim 74, wherein the fusion protein is abatacept.

76. A method for treating or preventing a hair-loss disorder in a mammalian subject in need thereof, the method comprising administering to the subject a therapeutic amount of a pharmaceutical composition comprising a fusion protein directed to an HLDGC protein, thereby treating or preventing a hair-loss disorder.

77. The method of claim 76, wherein the HLDGC protein is CTLA-4, IL-2, IL-21, IL-2RA/CD25, IKZF4, a HLA Region residing gene, PTGER4, PRDX5, STX17, NKG2D, ULBP6, ULBP3, HDAC4, CACNA2D3, IL-13, IL-6, CHCHD3, CSMD1, IFNG, IL-26, KIAA0350 (CLEC16A), SOCS1, ANKRD12, or PTPN2.

78. The method of claim 76, wherein the fusion protein is CTLA4-Ig.

79. The method of claim 78, wherein the fusion protein is abatacept.

80. The method of claim 76, wherein the hair-loss disorder comprises androgenetic alopecia, telogen effluvium, alopecia areata, telogen effluvium, tinea capitis, alopecia totalis, hypotrichosis, hereditary hypotrichosis simplex, or alopecia universalis.

81. A method for treating or preventing alopecia areata in a mammalian subject in need thereof, the method comprising administering to the subject a therapeutic amount of a pharmaceutical composition comprising CTLA4-Ig, thereby treating or preventing a hair-loss disorder.

82. The method of claim 81, wherein CTLA4-Ig is abatacept.

83. The method of claim 76 or 81, wherein the administering comprises a subcutaneous, intra-muscular, intra-peritoneal, or intravenous injection; an infusion; oral, nasal, or topical delivery; or a combination thereof.

84. The method of claim 76 or 81, wherein the administering comprises delivery of the composition to the epidermis or dermis of the subject.

85. The method of claim 84, wherein the epidermis or dermis is from the scalp.

86. The method of claim 76 or 81, wherein administering occurs daily, weekly, twice weekly, monthly, twice monthly, or yearly.
